(19) United States
(12) Patent Application Publication
(10) Pub. No.: US 2026/0060926 A1
(43) Pub. Date: Mar. 5, 2026

(54) NOVEL IONIZABLE LIPIDS AND LIPID NANOPARTICLES AND METHODS OF USING THE SAME

(71) Applicant: SAIL BIOMEDICINES, INC., Cambridge, MA (US)

(72) Inventors: Alessandra BARTOLOZZI, Cambridge, MA (US); John PROUDFOOT, Cambridge, MA (US); Arijit ADHIKARI, Cambridge, MA (US); Roman ERDMANN, Cambridge, MA (US); Dominick SALERNO, Cambridge, MA (US); Alaina HOWE, Cambridge, MA (US); Siddharth PATEL, Cambridge, MA (US); Feyisola OLATUNJI, Cambridge, MA (US)

(21) Appl. No.: 19/107,348

(22) PCT Filed: Aug. 31, 2023
(86) PCT No.: PCT/US2023/031669

§ 371 Date: Feb. 27, 2025

Related U.S. Application Data
(60) Provisional application No. 63/402,929, filed on Aug. 31, 2022.
(60) Provisional application No. 63/502,806, filed on May 17, 2023.

(51) Int. Cl. A61K 9/1271; A61K 9/51; A61K 31/7105; A61K 31/711; C07C 235/74; C07C 235/76; C07C 335/08; C07D 207/06; C07D 207/12; C07D 207/16; C07D 295/13; C07D 401/12; C07D 403/12; C07D 405/12; C07D 413/12; C12N 15/88

(52) U.S. Cl. CPC A61K 9/1271; A61K 9/5123; A61K 31/7105; A61K 31/711; C07C 235/74; C07C 235/76; C07C 335/08; C07D 207/06; C07D 207/12; C07D 207/16; C07D 295/13; C07D 401/12; C07D 403/12; C07D 405/12; C07D 413/12; C12N 15/88

(57) ABSTRACT

Novel ionizable lipids and lipid nanoparticles that can be used in the delivery of therapeutic cargos are disclosed.

### CROSS-REFERENCE TO RELATED APPLICATIONS

This application claims benefit of priority to U.S. Provisional Application No. 63/402,929 filed Aug. 31, 2022; and U.S. Provisional Application No. 63/502,806 filed May 17, 2023, which are herein incorporated by reference in their entirety.

### BACKGROUND

Lipid nanoparticles (“LNPs”) formed from ionizable amine-containing lipids can serve as therapeutic cargo vehicles for delivery of biologically active agents, such as coding RNAs (i.e., messenger RNAs (mRNAs), guide RNAs) and non-coding RNAs (i.e. antisense, siRNA), into cells. LNPs can facilitate delivery of oligonucleotide agents across cell membranes and can be used to introduce components and compositions into living cells.

Biologically active agents that are particularly difficult to deliver to cells include proteins, nucleic acid-based drugs, and derivatives thereof, particularly drugs that include relatively large oligonucleotides, such as mRNA or guide RNA. Compositions for delivery of promising mRNA therapy or editing technologies into cells, such as for delivery of CRISPR/Cas9 system components, have become of particular interest.

With the advent of the recent pandemic, messenger RNA therapy has become an increasingly important option for treatment of various diseases, including for viral infectious diseases and for those associated with deficiency of one or more proteins. Compositions with useful properties for in vitro and in vivo delivery that can stabilize and/or deliver RNA components, have also become of particular interest.

There thus continues to be a need in the art for novel lipid compounds to develop lipid nanoparticles or other lipid delivery mechanisms for therapeutics delivery. This invention answers that need.

### SUMMARY OF THE INVENTION

Disclosed herein are novel ionizable lipids that can be used in combination with at least one other lipid component, such as neutral lipids, cholesterol, and polymer conjugated lipids, to form lipid nanoparticle compositions. The lipid nanoparticle compositions may be used to facilitate the intracellular delivery of therapeutic nucleic acids in vitro and/or in vivo.

Disclosed herein are ionizable amine-containing lipids useful for formation of lipid nanoparticle compositions. Such LNP compositions may have properties advantageous for delivery of nucleic acid cargo, such as delivery of coding and non-coding RNAs to cells. Methods for treatment of various diseases or conditions, such as those caused by infectious entities and/or insufficiency of a protein, using the disclosed lipid nanoparticles are also provided.

Disclosed below are lipids, particularly ionizable lipids having specific tail groups (e.g., geminal, i.e., gem-di, functional groups bonded to the same carbon next to a biodegradable group, E).

### Tail Groups

Certain aspect of the invention relates to a lipid comprising at least one head group and at least one tail group of formula (T)

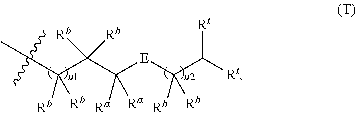

a pharmaceutically acceptable salt thereof, or a stereoisomer of any of the foregoing,
wherein:
E is a biodegradable group;
R^{a} is each independently for each occurrence C₁-C₅ branched or unbranched alkyl, C₂-C₅ branched or unbranched alkenyl, or C₂-C₅ branched or unbranched alkynyl, optionally interrupted with heteroatom or substituted with OH, SH, halogen, or NR⁷, wherein each R⁷ is independently H, alkyl, alkenyl, cycloalkyl, hydroxyalkyl, or aminoalkyl, or cycloalkyl or substituted cycloalkyl;
R^{b} is each independently for each occurrence H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl; C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
[symbol] represents the bond connecting the tail group to the head group; and
wherein the lipid has a pKa from about 4 to about 8.

As used herein, the term “biodegradable” refers to a group that include one or more bonds that may undergo bond breaking reactions in a biological environment, e.g., in an organism, organ, tissue, cell, or organelle. For example, the biodegradable group may be metabolizable by the body of a mammal, such as a human (e.g., by hydrolysis). Some groups that contain a biodegradable bond include, for example, but are not limited to, esters, dithiols, and oximes. Non-limiting examples of biodegradable groups are —OC(O)—, —C(O)O—, —SC(O)—, —C(O)S—, —OC(S)—, —C(S)O—, —S—S—, —C(R₅)═N—, —N═C(R₅)—, —C(R₅)═N—O—, —O—N═C(R₅)—, —C(O)(NR₅)—, —N(R₅)C(O)—, —C(S)(NR₅)—, —N(R₅)C(O)—, —N(R₅)C(O)N(R₅)—, —OC(O)O—, —OSi(R₅)₂O—, —C(O)(CR₃R₄)C(O)O—, or —OC(O)(CR₃R₄)C(O)—. Each R₅ is independently H, alkyl, alkenyl, cycloalkyl, hydroxyalkyl, or aminoalkyl; each R₃ and R₄ are independently branched or unbranched C₁-C₁₅ branched or unbranched alkyl, C₁-C₁₅ branched or unbranched alkenyl, or cycloalkyl or substituted cycloalkyl.

In some embodiments, R^{a} is each independently C₁-C₅ branched or unbranched alkyl, C₂-C₅ branched or unbranched alkenyl, or C₂-C₅ branched or unbranched alkynyl.

In some embodiments, R^{b} is each independently H, C₁-C₁₆ branched or unbranched alkyl or C₁-C₁₆ branched or unbranched alkenyl.

In some embodiments, R^{a} is each independently C₁-C₃ branched or unbranched alkyl. In one embodiment, each R^{a} is methyl.

In some embodiments, R^{b} is each independently H or C₁-C₃ branched or unbranched alkyl.

In some embodiments, E is —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —S—S—, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl, and r is 1, 2, 3, 4, or 5.

In some embodiments, E is —OC(O)—, —C(O)O—, —N(R⁷)C(O)—, or —C(O)N(R⁷)—, wherein R⁷ is independently H, alkyl, alkenyl, cycloalkyl, hydroxyalkyl, or aminoalkyl.

In one embodiment, E is —C(O)O—. In one embodiment, E is —OC(O)—. In one embodiment, E is N(R⁷)C(O)—. In one embodiment, E is or —C(O)N(R⁷)—.

In some embodiments, provided is a lipid comprising at least one head group and at least one tail group having a formula (TI) or (TI′):

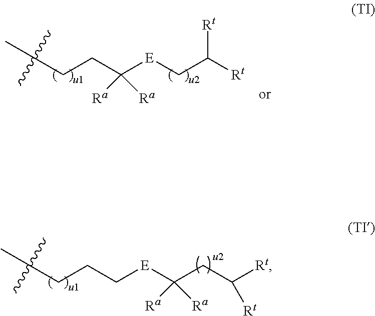

a pharmaceutically acceptable salt thereof, or a stereoisomer of any of the foregoing,
wherein:
E is each independently a biodegradable group;
R^{a} is each independently for each occurrence C₁-C₅ branched or unbranched alkyl, C₂-C₅ branched or unbranched alkenyl, or C₂-C₅ branched or unbranched alkynyl, optionally interrupted with heteroatom or substituted with OH, SH, halogen, or NR⁷, wherein each R⁷ is independently H, alkyl, alkenyl, cycloalkyl, hydroxyalkyl, or aminoalkyl; or cycloalkyl or substituted cycloalkyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
[symbol] represents the bond connecting the tail group to the head group; and
wherein the lipid has a pKa from about 4 to about 8.

In some embodiments, R^{a} is each independently for each occurrence C₁-C₅ branched or unbranched alkyl, C₂-C₅ branched or unbranched alkenyl, or C₂-C₅ branched or unbranched alkynyl. In some embodiments, R^{a} is each independently for each occurrence C₁-C₃ branched or unbranched alkyl. In one embodiment, each R^{a} is methyl.

In some embodiments, E is each independently —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —S—S—, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl, and r is 1, 2, 3, 4, or 5.

In some embodiments, E is each independently —OC(O)—, —C(O)O—, —N(R⁷)C(O)—, or —C(O)N(R⁷)—, wherein R⁷ is independently H, alkyl, alkenyl, cycloalkyl, hydroxyalkyl, or aminoalkyl.

In some embodiments, E is each independently —C(O)O—. In some embodiments, E is each independently —OC(O)—. In some embodiments, E is each independently —N(R⁷)C(O)—, wherein R⁷ is independently H or methyl. In some embodiments, E is each independently —C(O)N(R⁷)—, wherein R⁷ is independently H or methyl.

In some embodiments, the lipid comprises at least one head group and at least one tail group of formula (TII):

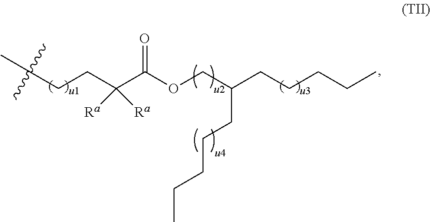

wherein u3 and u4 are each independently 1-7 (e.g., 0, 1, 2, 3, or 4). The definitions of other variables in (TII) are the same as those defined above in (TI).

In some embodiments, the lipid comprises at least one head group and at least one tail group of formula (TIII):

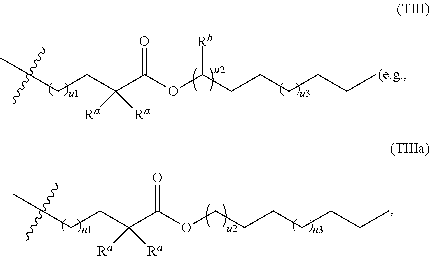

wherein u3 is 0, 1, 2, 3, 4, 5, 6, or 7; and R^{b} is in each occasion independently H or C₁-C₄ alkyl. The definitions of other variables in (TIII) are the same as those defined above in (TI).

In some embodiments, the lipid comprises at least one head group and at least one tail group of formula (TIV):

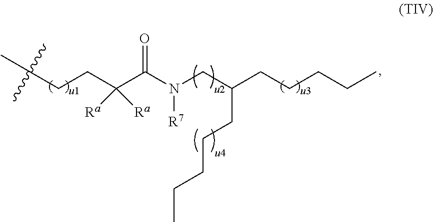

wherein u3 and u4 are each independently 1-7 (e.g., 0, 1, 2, 3, or 4). The definitions of other variables in (TIV) are the same as those defined above in (TI).

In some embodiments, the lipid comprises at least one head group and at least one tail group of formula (TV)

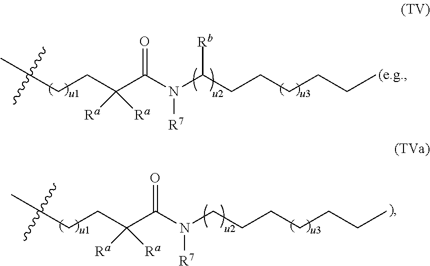

wherein u3 is 0, 1, 2, 3, 4, 5, 6, or 7; R⁷ is each independently H or methyl; and R^{b} is in each occasion independently H or C₁-C₄ alkyl. The definitions of other variables in (TV) are the same as those defined above in (TI).

In some embodiments, the lipid comprises at least one head group and at least one tail group of formula (TII′)

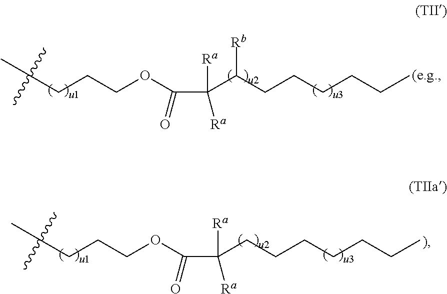

wherein u3 is 0, 1, 2, 3, 4, 5, 6, or 7; and R^{b} is in each occasion independently H or C₁-C₄ alkyl. The definitions of other variables in (TII′) are the same as those defined above in (TI′).

In some embodiments, the lipid comprises at least one head group and at least one tail group of formula (TII′)

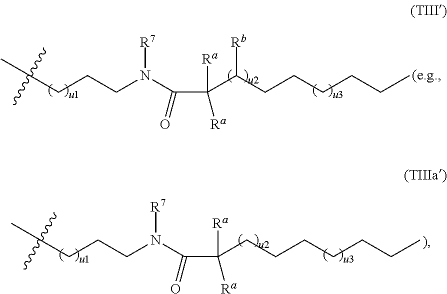

wherein u3 is 0, 1, 2, 3, 4, 5, 6, or 7; R⁷ is each independently H or methyl; and R^{b} is in each occasion independently H or C₁-C₄ alkyl. The definitions of other variables in (TIII′) are the same as those defined above in (TI′).

In some embodiments, the lipid comprises at least one tail group of the following formulas:

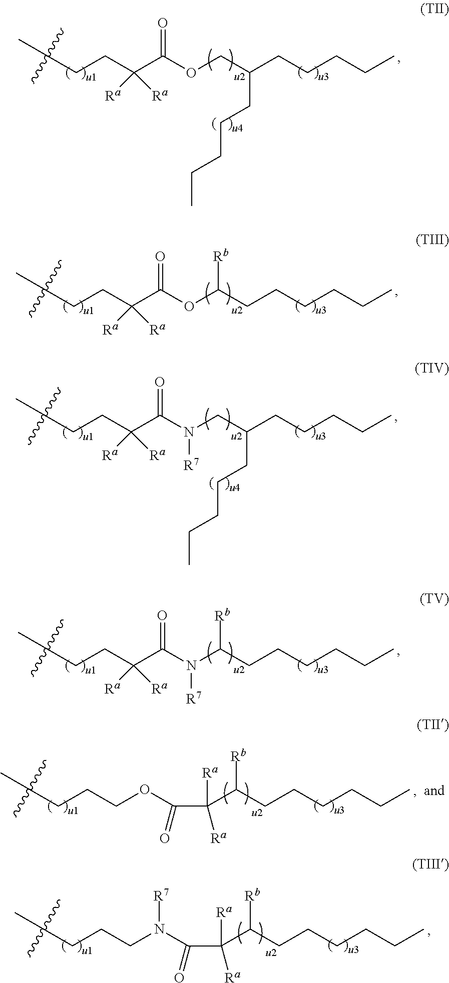

wherein
R⁷ is each independently H or methyl;
R^{b} is in each occasion independently H or C₁-C₄ alkyl;
u3 and u4 are each independently 0, 1, 2, 3, 4, 5, 6, or 7; and
wherein the lipid has a pKa from about 4 to about 8.

In some embodiments, the lipid comprises two or more tail groups that have a formula of (T), (TI), (TII), (TIII), (TIV), (TV), (TII′), and/or (TIII′), and each tail group may be the same or different.

In some embodiments, the lipid comprises three or more tail groups that have a formula of (T), (TI), (TII), (TIII), (TIV), (TV), (TI′), (TII′), and/or (TIII′), and each tail group may be the same or different.

In some embodiments, the lipid comprises four or more tail groups that have a formula of (T), (TI), (TII), (TIII), (TIV), (TV), (TI′), (TII′), and/or (TIII′), and each tail group may be the same or different.

In some embodiments, in any of the above formulas (T), (TI), (TII), (TII), (TIV), (TV), (TI′), (TII′), and (TIII′), R^{a} is methyl.

In some embodiments, in any of the above formulas (T), (TI), (TII), and (TIII), (TIV), (TV), (TI′), (TII′), and/or (TIII′), u1 is 3, 4, or 5.

In some embodiments, in any of the above formulas (T), (TI), (TII), and (TIII), (TIV), (TV), (TI′), (TII′), and/or (TIII′), u2 is 0, 1, 2, or 3.

In some embodiments, in any of the above formulas (T), (TI), (TII), and (TIII), (TIV), (TV), (TI′), (TII′), and/or (TIII′), u3 and u4 are each independently 1-7, for instance, u3 and u4 are each independently 1, 2, 3, or 4.

In some embodiments, the lipid comprises at least one tail of formula (TIII), wherein each R^{a} is methyl; R^{b} is in each occasion independently H, ethyl, or butyl; u1 is 3-5, u2 is 0-3, and u3 is 1-7 (e.g., 1-4).

In some embodiments, the lipid comprises at least one two tails of formula (TIII), wherein the two tails of formula (TIII) are the same or different. In some embodiments, the lipid comprises at least three tails of formula (TIII), wherein each tail may be the same or different.

In some embodiments, the lipid has four tails of formula (TIII), wherein each tail may be the same or different.

In some embodiments, in each tail of formula (TIII), each R^{a} is methyl, and u1 is 3, u2 is 2, and u3 is 4.

In some embodiments, the lipid comprises at least one tail of formula (TII), wherein each R^{a} is methyl, u1 is 3-5, u2 is 0-3, u3 is 1-4, and u4 is 1-4.

In some embodiments, the lipid has at least two tails of formula (TII), wherein the two tails of formula (TII) are the same or different. In some embodiments, the lipid comprises at least three tails of formula (TII), wherein each tail may be the same or different. In some embodiments, the lipid has four tails of formula (TII), wherein each tail may be the same or different.

In some embodiments, in each tail of formula (TII), each R^{a} is methyl, and variables u1, u2, u3, and u4 are one of the followings:
(i) u1 is 5, u2 is 3, and u3 and u4 are each 1;
(ii) u1 is 5, u2 is 0, and u3 and u4 are each 2;
(iii) u1 is 5, u2 is 0, and u3 and u4 are each 3;
(iv) u1 is 5, u2 is 0, and u3 and u4 are each 4;
(v) u1 is 5, u2 is 0, u3 is 4, and u4 is 2; or
(vi) u1 is 3, u2 is 3, and u3 and u4 are each 1.

In some embodiments, the lipid comprises at least one tail of formula (TIV), wherein each R^{a} is methyl, u1 is 3-5, u2 is 0-3, u3 is 1-4, and u4 is 1-4.

In some embodiments, the lipid comprises at least two tails of formula (TIV), wherein each tail may be the same or different. In some embodiments, the lipid comprises at least three tails of formula (TIV), wherein each tail may be the same or different. In some embodiments, the lipid comprises at least four tails of formula (TIV), wherein each tail may be the same or different.

In some embodiments, the lipid comprises at least two tails of formula (TV), wherein each tail may be the same or different. In some embodiments, the lipid comprises at least three tails of formula (TV), wherein each tail may be the same or different. In some embodiments, the lipid comprises at least four tails of formula (TV), wherein each tail may be the same or different.

In some embodiments, the lipid has at least two tails of formula (TII′), wherein each tail may be the same or different. In some embodiments, the lipid has at least three tails of formula (TII′), wherein each tail may be the same or different. In some embodiments, the lipid has at least four tails of formula (TII′), wherein each tail may be the same or different.

In some embodiments, the lipid has at least two tails of formula (TIII′), wherein each tail may be the same or different. In some embodiments, the lipid has at least three tails of formula (TIII′), wherein each tail may be the same or different. In some embodiments, the lipid has at least four tails of formula (TIII′), wherein each tail may be the same or different.

In some embodiments, the lipid has at least one tail selected from the group consisting of formula (TII), (TIII), and (TII′).

In some embodiments, the lipid has at least one tail selected from the group consisting of (TIV), (TV), and (TIII′).

In some embodiments, the lipid has at least one tail selected from the group consisting of formula (TII), (TIII), and (TII′), and at least one tail selected from the group consisting of (TIV), (TV), and (TIII′).

In some embodiments, the lipid has at least two tails selected from the group consisting of (TII), (TIII), and (TII′).

In some embodiments, the lipid has at least two tails selected from the group consisting of (TIV), (TV), and (TIII′).

In some embodiments, the lipid has at least two tails selected from the group consisting of formula (TII), (TIII), and (TII′), and at least one tail selected from the group consisting of (TIV), (TV), and (TIII′).

In some embodiments, the lipid has at least one tail selected from the group consisting of formula (TII), (TIII), and (TII′), and at least two tails selected from the group consisting of (TIV), (TV), and (TIII′).

In some embodiments, the lipid has at least two tails selected from the group consisting of formula (TII), (TIII), and (TII′), and at least two tails selected from the group consisting of (TIV), (TV), and (TIII′).

In some embodiments, the lipid has at least three tails selected from the group consisting of (TII), (TIII), and (TII′).

In some embodiments, the lipid has at least three tails selected from the group consisting of (TIV), (TV), and (TIII′).

In some embodiments, the lipid has at least three tails selected from the group consisting of formula (TII), (TIII), and (TII′), and at least one tail selected from the group consisting of (TIV), (TV), and (TIII′).

In some embodiments, the lipid has at least one tail selected from the group consisting of formula (TII), (TIII), and (TII′), and at least three tails selected from the group consisting of (TIV), (TV), and (TIII′).

In some embodiments, the lipid has at least one tail of formula (TII) or (TIII), and at least one tail of formula (TIV) or (TV). In some embodiments, the lipid has at least two tails of formula (TII) or (TIII), and at least two tails of formula (TIV) or (TV).

In some embodiments, the lipid has at least one tail of formula (TII) or (TIII), and at least one tail of formula (TII′) or (TIII′). In some embodiments, the lipid has at least two tails of formula (TII) or (TIII), and at least two tails of formula (TII′) or (TIII′).

In some embodiments, the lipid has at least one tail of formula (TIV) or (TV), and at least one tail of formula (TII′) or (TIII′). In some embodiments, the lipid has at least two tails of formula (TIV) or (TV), and at least two tails of formula (TII′) or (TIII′).

In some embodiments, the lipid has at least one tail of formula (TII) and at least one tail of formula (TIII). In some embodiments, the lipid has at least two tails of formula (TII) and at least two tails of formula (TIII). In some embodiments, in each tail of formula (TII) or formula (TIII), R^{a} is methyl, u1 is 3-5, u2 is 0-2, u3 is 1-4, and u4 is 1-4.

In some embodiments, the lipid has at least one tail of formula (TII) and/or at least one tail of formula (TIII); the lipid further comprises at least one tail that does not have a formula (T), (TI), (TII), (TIII), (TIV), (TV), (TII′), and/or (TIII′). That is to say, the lipid further comprises at least one tail that does not contain a gem-di functional groups bonded to the same carbon next to E (e.g., —C(O)O—).

In some embodiments, the lipid further comprises at least one tail that does not have a formula (T), (TI), (TII), (TIII), (TIV), (TV), (TI′), (TII′), and/or (TIII′). That is to say, the lipid further comprises at least one tail that does not contain a gem-di functional groups bonded to the same carbon next to E.

In some embodiments, the lipid further comprises at least one tail of formula (TNG-1):

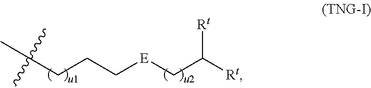

wherein
E is each independently a biodegradable group as described herein, e.g., —OC(O)—, —C(O)O—, —N(R⁷)C(O)—, —S—S—, or —C(O)N(R⁷)—;
u1 and u2 are each independently 0, 1, 2, 3, 4, 5, 6, or 7; and
R⁷ is independently H, alkyl, alkenyl, cycloalkyl, hydroxyalkyl, or aminoalkyl.

In some embodiments, the at least one tail of formula (TNG-I) can be represented by

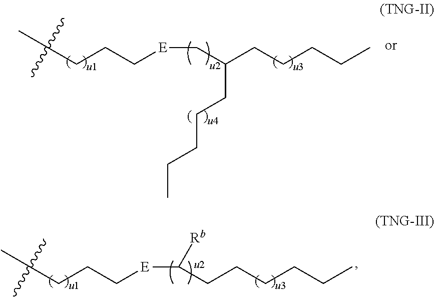

wherein
   u3 and u4 are each independently 0, 1, 2, 3, 4, 5, 6, or 7; and
   R^{b} is in each occasion independently H or C₁-C₄ alkyl.

All the embodiments above regarding the definitions of E, R^{b}, R^{t}, u1, u2, u3 and u4, as described above relating to the tail group containing a gem-di functional group bonded to the same carbon next to E, having a formula (T), (TI), (TII), (TIII), (TIV), (TV), (TII′), or (TIII′), are also applicable to the tail group that does not contain a gem-di functional groups bonded to the same carbon next to E, having a formula (TNG-I), (TNG-II), or (TNG-III).

In some embodiments, the lipid further comprises at least two tails that do not have a formula (T), (TI), (TII), (TIII), (TIV), (TV), (TI′), (TII′), and/or (TIII′). In some embodiments, the lipid comprises two tail groups of formula (TNG-II) or (TNG-III), and wherein each tail group may be the same or different,

In some embodiments, the lipid further comprises at least three tails that do not have a formula (T), (TI), (TII), (TIII), (TIV), (TV), (TI′), (TII′), and/or (TIII′). In some embodiments, the lipid comprises three tail groups of formula (TNG-II) or (TNG-III), and wherein each tail group may be the same or different,

### Head Groups

The head group of the lipid can be any amine-containing head group for a typical ionizable lipid.

In some embodiments, the head group of the lipid has a structure of formula (HA-I):

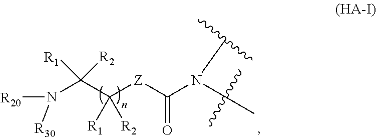

wherein:
   R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
R₂₀ and R₃₀, together with the adjacent N atom, form a 3 to 7 membered heterocylic or heteroaromatic ring containing one or more heteroatoms, optionally substituted with one or more OH, SH, halogen, alkyl, or cycloalkyl groups;
each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
each of R₁₀ and R₁₁ is independently H, C₁-C₃ branched or unbranched alkyl, C₂-C₃ branched or unbranched alkenyl; or R₁₀ and R₁₁ together form a heterocyclic ring;
n is 0, 1, 2, 3 or 4;
Z is absent, O, S, or NR₁₂, wherein R₁₂ is H or C₁-C₇ branched or unbranched alkyl;
provided that when Z is not absent, the adjacent R₁ and R₂ cannot be OH, NR₁₀R₁₁, SH.

In some embodiments, R₂₀ and R₃₀ together with the adjacent N atom form a 3 to 7 membered heterocylic or heteroaromatic ring containing one or more heteroatoms, optionally substituted with one or more OH, SH, halogen, alkyl, or cycloalkyl groups.

In some embodiments, the head group of the lipid has a structure of formula (HA-IA):

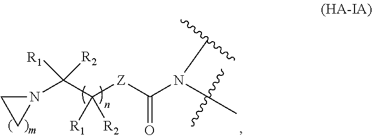

wherein:
   each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ are taken together to form a cyclic ring;
   each of R₁₀ and R₁₁ is independently H, C₁-C₃ branched or unbranched alkyl, C₂-C₃ branched or unbranched alkenyl; or R₁₀ and R₁₁ are taken together to form a heterocyclic ring;
   m is 1, 2, 3, 4, 5, 6, 7 or 8;
   n is 0, 1, 2, 3 or 4;
   Z is absent, O, S, or NR₁₂, wherein R₁₂ is H or C₁-C₇ branched or unbranched alkyl;
   provided that when Z is not absent, the adjacent R₁ and R₂ cannot be OH, NR₁₀R₁₁, SH; and
   [symbol] represents the bond connecting the head group to the tail group.

In some embodiments, m is 1, 2, 3, or 4.

In some embodiments, the head group of the lipid has a structure of formula (HA-III):

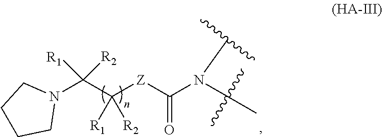

wherein Z is absent, O, S, or NR₁₂; and R₁₂ is C₁-C₇ alkyl. The definitions of other variables in (HA-III) are the same as those defined above in (HA-I).

In some embodiments, in any of the above formulas such as (HA-I), (HA-IA), or (HA-III), Z is absent, O, S, or NH.

In some embodiments, in any of the above formulas such as (HA-I), (HA-IA), or (HA-III), each R₁ and R₂ are H.

In some embodiments, in any of the above formulas such as (HA-I), (HA-IA), or (HA-III), n is 0, 1, or 2.

In some embodiments, the head group has a structure of:

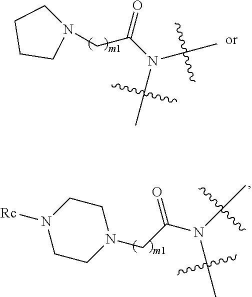

wherein:
Rc is H or alkyl, optionally substituted with OH; and
m1 is 1, 2, or 3.

In some embodiments, the head group of the lipid has a structure of formula (HA-V):

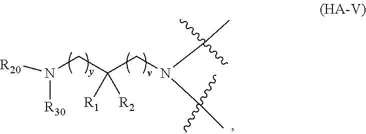

wherein:
   R₁ is H, C₁-C₃ alkyl, OH, halogen, SH, or NR₁₀R₁₁;
   R₂ is OH, halogen, SH, or NR₁₀R₁₁; or R₁ and R₂ can be taken together to form a cyclic ring;
   R₁₀ and R₁₁ are each independently H or C₁-C₃ alkyl; or R₁₀ and R₁₁ can be taken together to form a heterocyclic ring;
   R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
   each of v and y is independently 1, 2, 3, or 4.
   In some embodiments, the head group of the lipid has a structure of formula (HA-VI):

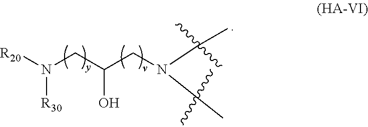

The definitions of all variables in (HA-VI) are the same as those defined above in (HA-V).

In some embodiments, in any of the above formulas such as (HA-V) or (HA-VI), each R₂₀ and R₃₀ are independently C₁-C₃ alkyl. In one embodiment, each R₂₀ and R₃₀ are independently methyl.

In some embodiments, the head group of the lipid has a structure of formula (HA-VII):

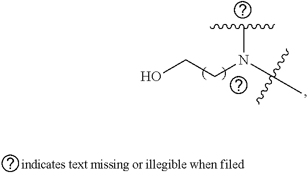

wherein u20 is 1, 2, 3, 4 or 5.

In some embodiments, the head group of the lipid has a structure of formula (HB-I):

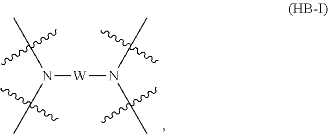

wherein W is

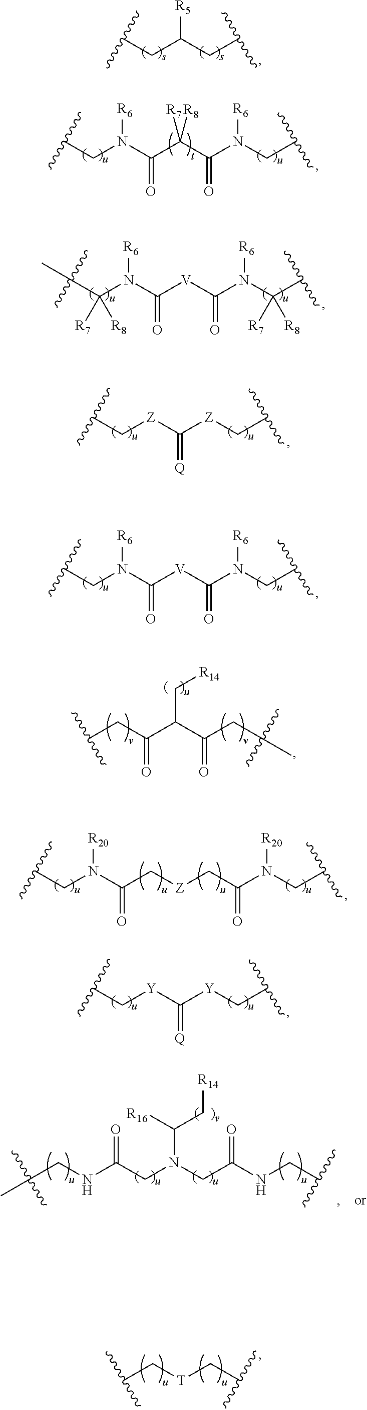

wherein
   R₅ is OH, SH, (CH₂)ₛOH, or NR₁₀R₁₁;
   each R₆ is independently H, C₁-C₃ branched or unbranched alkyl, C₂-C₃ branched or unbranched alkenyl, or cycloalkyl;
   each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or R₇ and R₈ are taken together to form a ring;
   each R₂₀ is independently H, or C₁-C₃ branched or unbranched alkyl;
   R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
   R₁₆ is H, ═O, ═S, or CN;
   each of s, u, and t is independently 1, 2, 3, 4, or 5;
   each v is independently 0, 1, 2, 3, 4, or 5;
   each Y is a divalent heterocyclic;
   each Z is independently absent, O, S, or NR₁₂, wherein R₁₂ is H, C₁-C₇ branched or unbranched alkyl, or C₂-C₇ branched or unbranched alkenyl;
   Q is O, S, CH₂, or NR₁₃, wherein each R₁₃ is H, C₁-C₅ alkyl;
   V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
   T is —NHC(O)O—, —OC(O)NH—, or a divalent heterocyclic.

In some embodiments, R₅ is OH or (CH₂)ₛOH; and s is 1 or 2.

In some embodiments, each R₆, R₇, and R₈ are independently H or C₁-C₃ alkyl.

In some embodiments, each of u and t is independently 1, 2, or 3.

In some embodiments, each v is independently 0, 1, 2, or 3.

In some embodiments, R₁₆ is H or ═O.

In some embodiments, each Z is independently absent, O, or NR₁₂, wherein R₁₂ is H or C₁-C₃ alkyl.

In some embodiments, T is a divalent heterocylic.

In some embodiments, Q is O or CH₂.

In some embodiments, V is C₂-C₆ alkylene or C₂-C₆ alkenylene.

In some embodiments, the heterocyclic (or divalent heterocyclic) is a piperazine, piperazine dione, piperazine-2,5-dione, piperidine, pyrrolidine, piperidinol, dioxopiperazine, bis-piperazine, aromatic or heteroaromatic.

In some embodiments, in formula (HB-I), W is

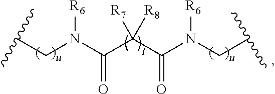

wherein:
each R₆, R₇, and R₈ are independently H or methyl; and
each of u and t is independently 1, 2, or 3.

In some embodiments, in formula (HB-I), W is

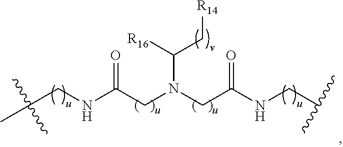

wherein:
R₁₆ is H or ═O;
R₁₄ is a nitrogen-containing 5- or 6-membered heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H or C₁-C₃ alkyl; and
each of u and v is independently 1, 2, or 3.

In some embodiments, in formula (HB-I), W is

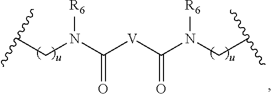

wherein:
each R₆ is independently H or methyl;
each u is independently 1, 2, or 3; and
V is C₂-C₆ alkylene or C₂-C₆ alkenylene.

In some embodiments, in formula (HB-I), W is

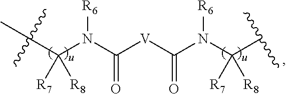

wherein:
each R₆ is independently H or methyl;
each R₇ is independently H;
each R₈ is methyl;
each u is independently 1, 2, or 3; and
V is C₂-C₆ alkylene or C₂-C₆ alkenylene.

In some embodiments, in formula (HB-I), W is

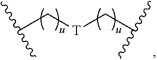

wherein:
each u is independently 1, 2, or 3; and
T is a divalent nitrogen-containing 5- or 6-membered heterocyclic.

In some embodiments, in formula (HB-I), W is

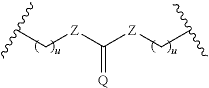

wherein:
each u is independently 1, 2, or 3;
Q is O;
each Z is independently NR₁₂; and
R₁₂ is H or C₁-C₃ alkyl.

In some embodiments, the head group has the structure of:

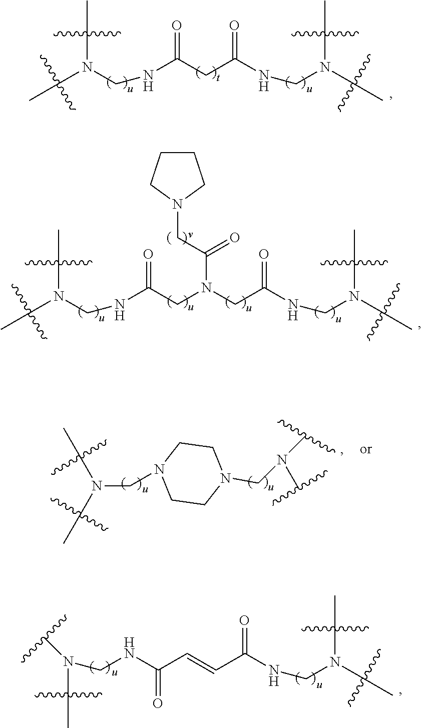

wherein each of u and t is independently 1 or 2.

In some embodiments, the head group of the lipid has a structure of formula (HC-I):

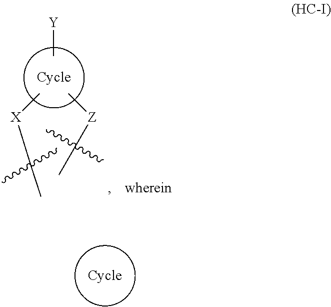

is cyclic or heterocyclic moiety;
Y is alkyl, hydroxy, hydroxyalkyl,

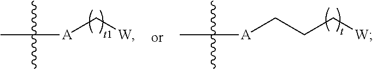

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—;
each of X and Z is independently absent, —O—, —C(O)—, —N(R⁷)—, alkylene, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t is 0, 1, 2, or 3;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl.

In some embodiments, W is hydroxyl, substituted or unsubstituted hydroxyalkyl, or one of the following moieties:

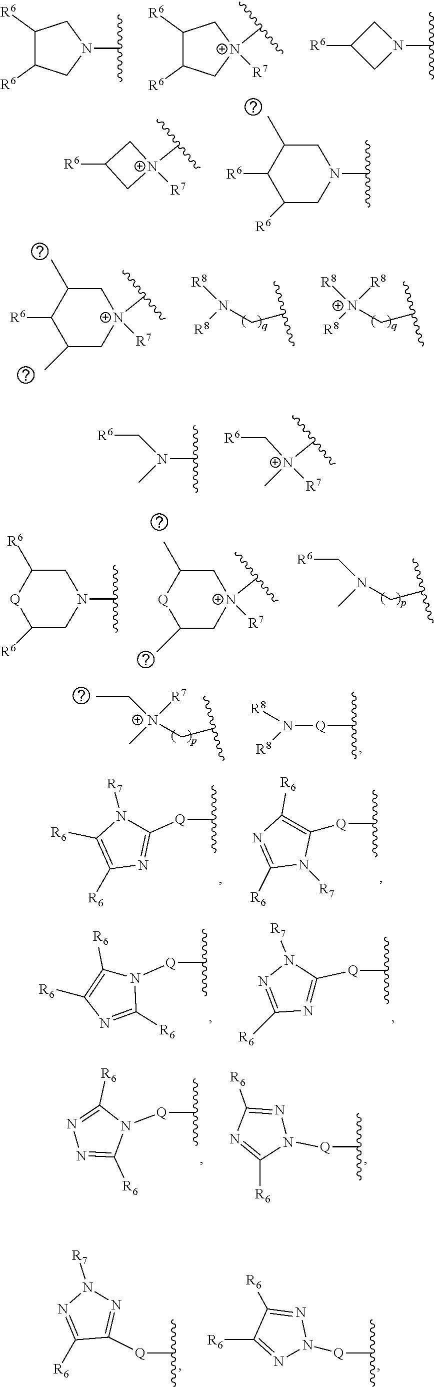

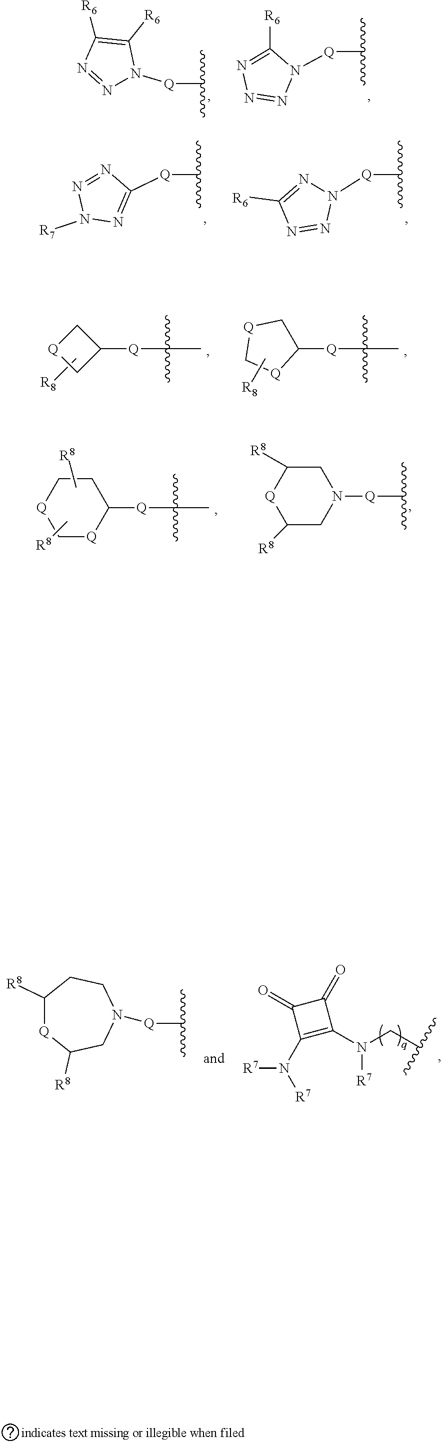

wherein
   each Q is independently absent, —O—, —C(O)—, —C(S)—, —C(O)O—, —(CH₂)_{q}—C(R⁷)₂—, —C(O)N(R⁷)—, —C(S)N(R⁷)—, or —N(R⁷);
   R⁶ is independently H, alkyl, hydroxyl, hydroxyalkyl, alkoxy, —O-alkylene-O-alkyl, —O-alkylene-N(R⁷)₂, amino, alkylamino, aminoalkyl, thiol, thiolalkyl, or N⁺(R⁷)₃-alkylene-Q-;
   each R⁸ is independently H, alkyl, hydroxyalkyl, amino, aminoalkyl, alkylamino, thiol, or thiolalkyl, heterocyclyl, heteroaryl, or two R⁸ together with the nitrogen atom may form a ring, optionally substituted with one or more alkyl, hydroxy, hydroxyalkyl, alkoxy, alkylaminoalkyl, alkylamino, aminoalkyl;
   q is 0, 1, 2, 3, 4, or 5; and
   p is 0, 1, 2, 3, 4, or 5.

In some embodiments, the head group of the lipid has a structure of formula (HC-IA):

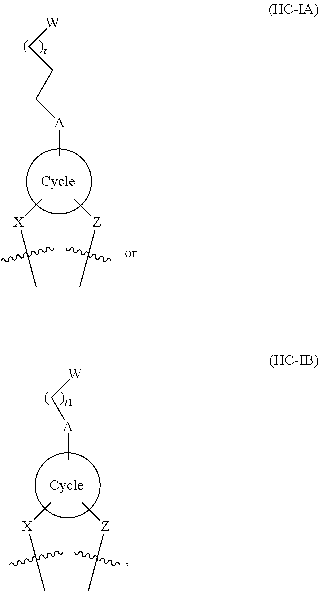

The definitions of all variables in (HC-IA) or (HC-IB) are the same as those defined above in (HC-I).

In some embodiments, in any of the above formula such as (HC-I), (HC-IA), or (HC-IB), [symbol] is a 5- to 7-membered, monocyclic ring. In some embodiments, [symbol] is a 5- to 7-membered, monocyclic, cycloalkane ring. In some embodiments, [symbol] is a 5- to 7-membered, monocyclic, heterocycle ring.

In some embodiments, in any of the above formula such as (HC-I), (HC-IA), or (HC-IB), [symbol] is a bicyclic or tricyclic ring, i.e., containing two or more rings, such as fused rings.

In some embodiments, in any of the above formula such as (HC-I), (HC-IA), or (HC-IB), [symbol] has a structure of formula

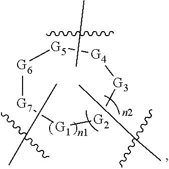

wherein:
   each of G₁, G₂, G₃, G₄, G₅, G₆, and G₇ is independently C(R′)(R″), O, or N, provided that no more than two of G₁-G₇ are O or N;
   R′ and R″ are each independently absent, H, alkyl, or two R′ from the two neighboring G together form a second 5- to 7-membered cyclic or heterocylic ring; and
   n₁ and n₂ are each independently 0 or 1.

In some embodiments, in any of the above formula such as (HC-I), (HC-IA), or (HC-IB), [symbol] is selected from pyrrolidine, piperidine, piperazine, cyclohexane, cyclopentane, tetrahydrofuran; tetrahydropyran; morpholine, and dioxane.

In some embodiments, in any of the above formula such as (HC-I), (HC-IA), or (HC-IB), [symbol] is selected from the group consisting of

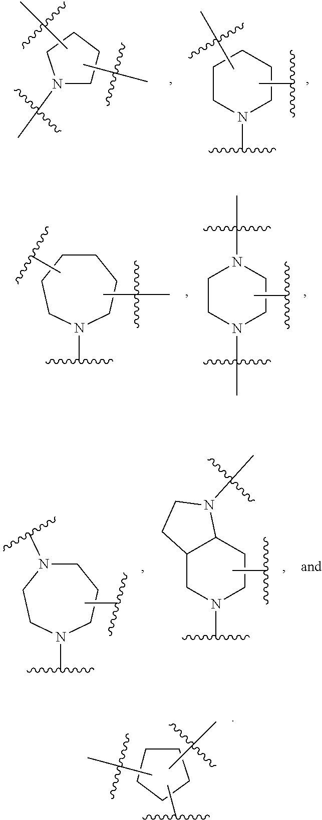

In some embodiments, in any of the above formula such as (HC-I), (HC-IA), or (HC-IB), [symbol] is selected from the group consisting of

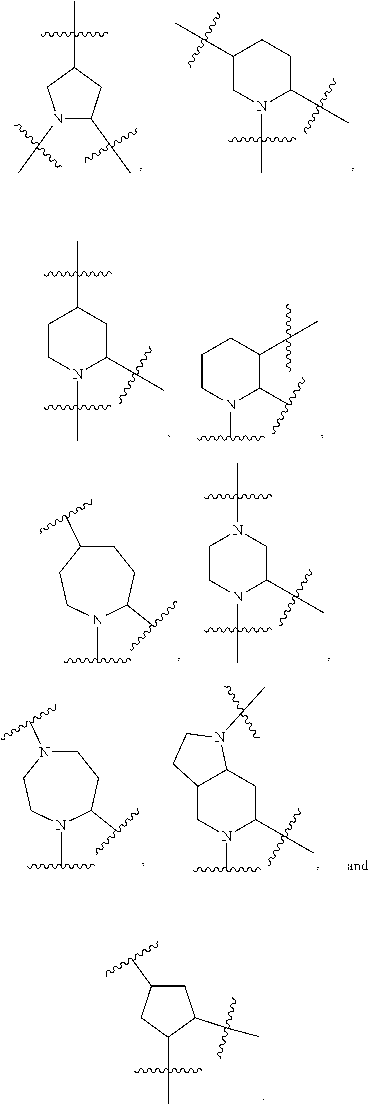

In some embodiments the head group of the lipid has a structure of formula (HC-IIA):

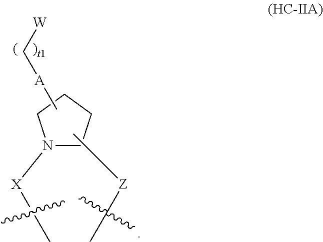

Each R⁷ is independently H, C₁-C₃ branched or unbranched alkyl, C₂-C₃ branched or unbranched alkenyl, cycloalkyl, hydroxyalkyl, or aminoalkyl. The definitions of all other variables in (HC-IIA) are the same as those defined above in (HC-I).

In some embodiments, the head group of the lipid has a structure of formula (HC-IIA′):

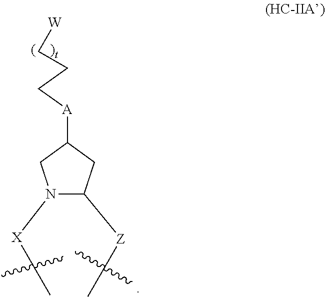

Each R⁷ is independently H, C₁-C₃ branched or unbranched alkyl, C₂-C₃ branched or unbranched alkenyl, cycloalkyl, hydroxyalkyl, or aminoalkyl. The definitions of all other variables in (HC-IIA′) are the same as those defined above in (HC-I).

In some embodiments, the head group of the lipid has a structure of formula (HC-IIC):

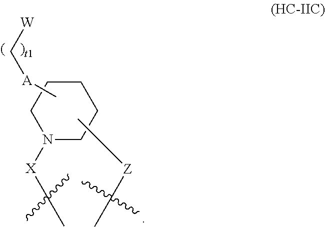

Each R⁷ is independently H, C₁-C₃ branched or unbranched alkyl, C₂-C₃ branched or unbranched alkenyl, cycloalkyl, hydroxyalkyl, or aminoalkyl. The definitions of all other variables in (HC-IIC) are the same as those defined above in (HC-I).

In some embodiments, the head group of the lipid has a structure of formula (HC-IIC′):

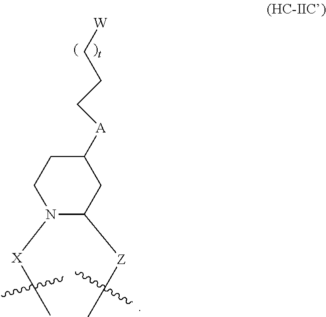

Each R⁷ is independently H, C₁-C₃ branched or unbranched alkyl, C₂-C₃ branched or unbranched alkenyl, cycloalkyl, hydroxyalkyl, or aminoalkyl. The definitions of all other variables in (HC-IIC′) are the same as those defined above in (HC-I).

In some embodiments, in any of the above formulas such as (HC-I), (HC-IA), (HC-IB), (HC-TIA), (HC-IIA′), (HC-IIC), and (HC-ITC′), X is absent, —O—, or —C(O)—.

In some embodiments, in any of the above formulas such as (HC-I), (HC-IA), (HC-IB), (HC-IIA), (HC-IIA′), (HC-IIC), and (HC-IIC′), Z is —O—, —C(O)O—, or —OC(O)—.

In some embodiments, the head group of the lipid a structure of one of the following formulas:

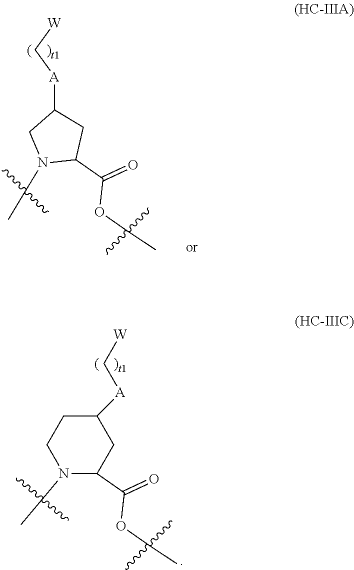

The definitions of all variables are the same as those defined above in (HC-I).

In some embodiments, the head group of the lipid a structure of one of the following formulas:

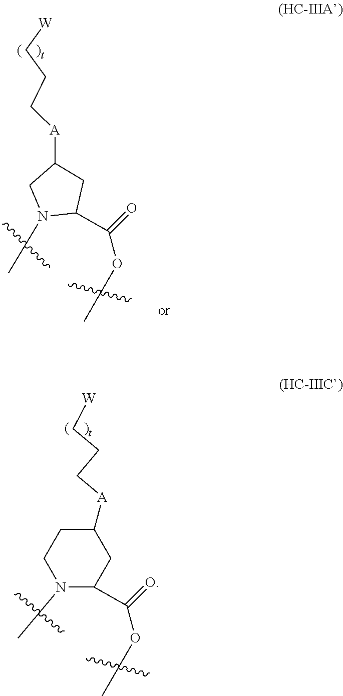

The definitions of all variables are the same as those defined above in (HC-I).

In some embodiments, in any of the above formulas such as (HC-I), (HC-IA), (HC-IB), (HC-IIA), (HC-IIA′), (HC-IIC), (HC-IIC′), (HC-IIIA), (HC-IIIA′), (HC-IIIC), and (HC-IIIC′), A is absent, —O—, —N(R⁷)—, —C(O)N(R⁷)—, —N(R⁷)C(O)—, —OC(O)—, or —C(O)O—. In one embodiment, A is absent. In one embodiment, A is —O—. In one embodiment, A is —N(R⁷)—, wherein R⁷ is H or C₁-C₃ alkyl. In one embodiment, A is —OC(O)— or —C(O)O—. In one embodiment, A is —NHC(O)— or —C(O)NH—.

In some embodiments, the head group of the lipid a structure of one of the following formulas:

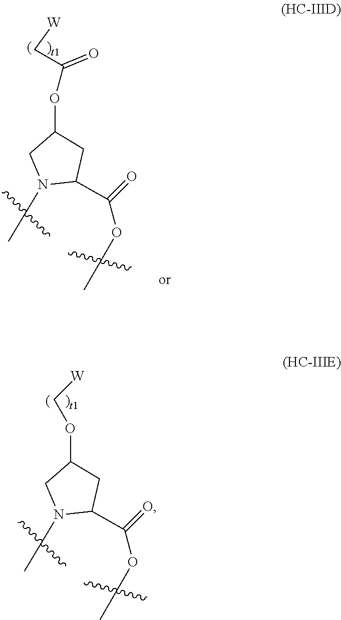

wherein t1 is 0, 1, 2, or 3. The definitions of the other variables in these formulas are the same as those defined above in (HC-I).

In some embodiments, the head group of the lipid a structure of one of the following formulas:

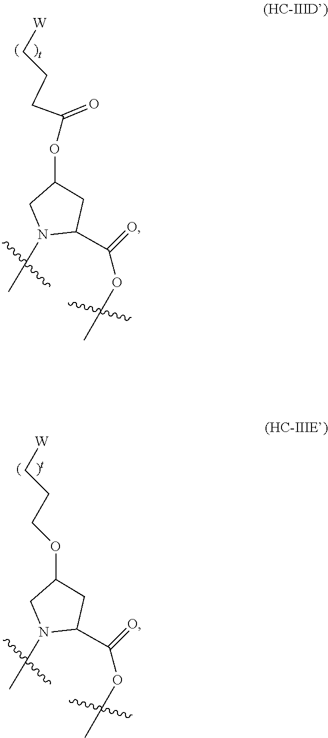

wherein the definitions of the variables in these formulas are the same as those defined above in (HC-I).

In some embodiments, in any of the above formulas such as (HC-I), (HC-IA), (HC-IB), (HC-IIA), (HC-IIA′), (HC-IIC), (HC-IIC′), (HC-IIIA), (HC-IIIA′), (HC-IIIC), (HC-IIIC′), (HC-IIID), (HC-IIID′), (HC-IIIE), and (HC-IIIE′), t is 0, 1, or 2.

In some embodiments, in any of the above formulas such as (HC-I), (HC-IA), (HC-IB), (HC-IIA), (HC-IIA′), (HC-IIC), (HC-IIC′), (HC-IIIA), (HC-IIIA′), (HC-IIIC), (HC-IIIC′), (HC-IIID), (HC-IIID′), (HC-IIIE), and (HC-IIIE′), W is OH.

In some embodiments, in any of the above formulas such as (HC-T), (HC-IA), (HC-IB), (HC-IIA), (HC-IIA′), (HC-IIC), (HC-IIC′), (HC-IIIA), (HC-IIIA′), (HC-IIIC), (HC-IIIC′), (HC-IIID), (HC-IIID′), (HC-IIIE), and (HC-IIIE′), W is OH,

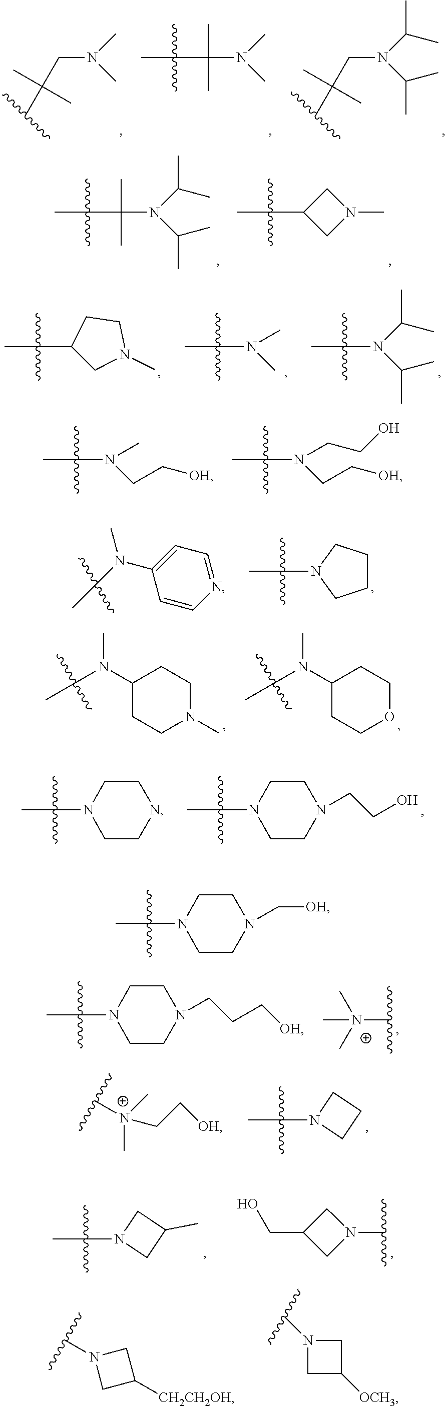

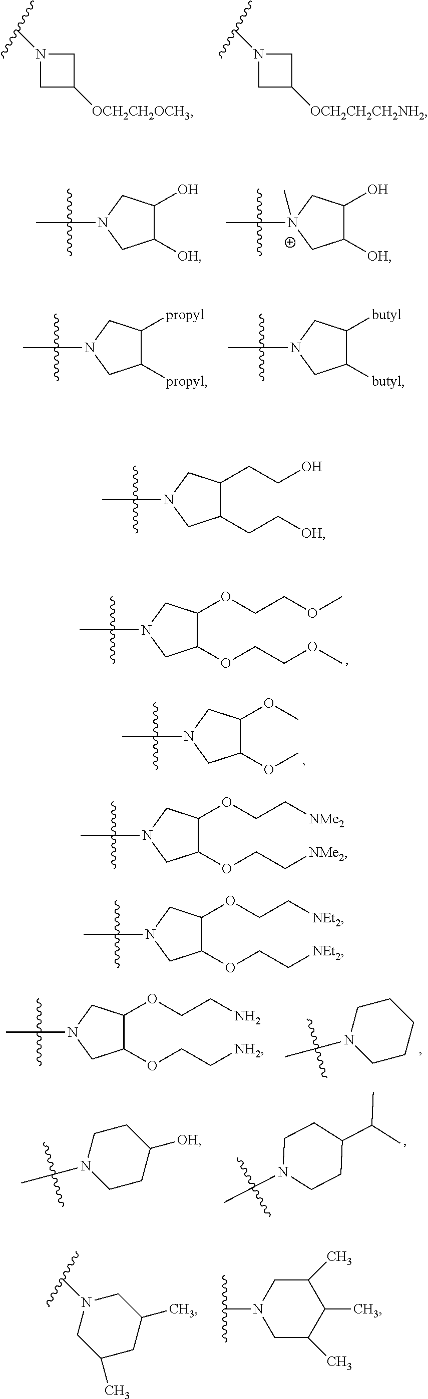

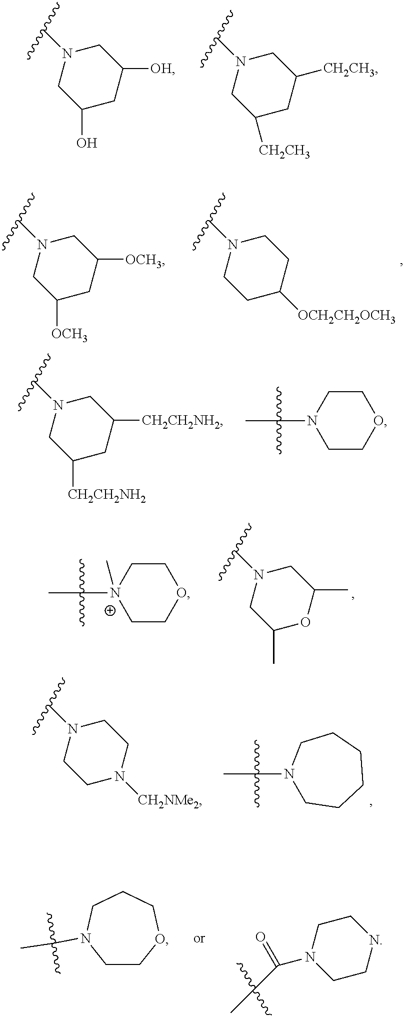

In some embodiments, W is

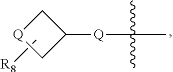

wherein Q is absent, —(CH₂)_{q}—C(R⁷)₂—, or —N(R⁷); q is 0 or 1; R⁷ is H or methyl; and each R⁸ is independently H or C₁-C₃ alkyl. In one embodiment, W is

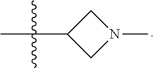 [SMILES: *C1CN(C)C1]

In some embodiments, W is

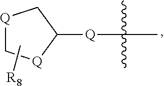

wherein Q is absent, —(CH₂)_{q}—C(R⁷)₂—, or —N(R⁷); q is 0 or 1; R⁷ is H or methyl; and each R⁸ is independently H or C₁-C₃ alkyl. In one embodiment, W is

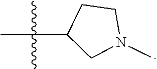 [SMILES: *C1CCN(C)C1]

In some embodiments, W is

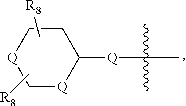

wherein Q is absent, —(CH₂)_{q}—C(R⁷)₂—, or —N(R⁷); q is 0 or 1; R⁷ is H or methyl; and each R⁸ is independently H or C₁-C₃ alkyl. In one embodiment, W is

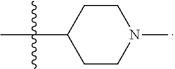 [SMILES: *C1CCN(C)CC1]

In some embodiments, W is

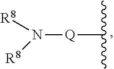

wherein Q is —(CH₂)_{q}—C(R⁷)₂—; q is 0 or 1; R⁷ is H or methyl; and each R⁸ is independently H or C₁-C₃ alkyl. In one embodiment, W is

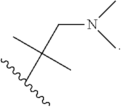 [SMILES: *C(C)(C)CN(C)C]

In one embodiment, W is

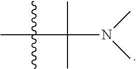 [SMILES: *C(C)(C)N(C)C]

In one embodiment, W is

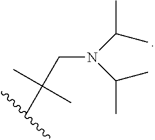 [SMILES: *C(C)(C)CN(C(C)C)C(C)C]

### In

### W is

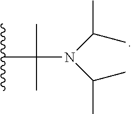 [SMILES: *C(C)(C)N(C(C)C)C(C)C]

In some embodiments, W is

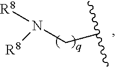

wherein q is 0, and each R⁸ is independently H, C₁-C₃ alkyl, hydroxyalkyl, heterocyclyl, or heteroaryl, optionally substituted with one or more alkyl. In one embodiment. W is

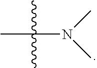 [SMILES: *N(C)C]

In one embodiment, W is

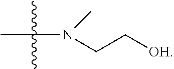 [SMILES: *N(C)CCO]

In one embodiment, W is

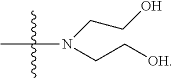 [SMILES: *N(CCO)CCO]

In one embodiment, W is

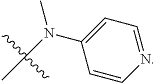 [SMILES: *N(C)c1ccncc1]

In one embodiment, W is

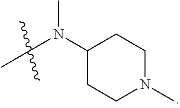 [SMILES: *N(C)C1CCN(C)CC1]

In one embodiment, W is

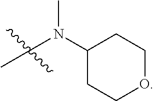 [SMILES: *N(C)C1CCOCC1]

In some embodiments, W is

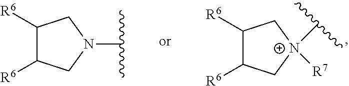

wherein each R⁶ is independently H, C₁-C₃ alkyl, hydroxyl, hydroxyalkyl, alkoxy, —O-alkylene-O-alkyl, or —O-alkylene-N(R⁷)₂ and each R⁷ is independently H or C₁-C₃ alkyl. In one embodiment, W is

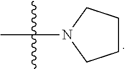 [SMILES: *N1CCCC1]

In one embodiment, W is

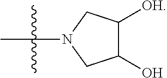 [SMILES: *N1CC(O)C(O)C1]

In one embodiment, W is

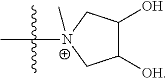 [SMILES: *[N+]1(C)CC(O)C(O)C1]

In one embodiment, W is

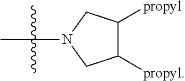

In one embodiment, W is

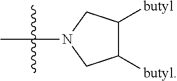

In one embodiment, W is

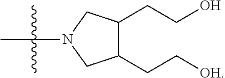 [SMILES: *N1CC(CCO)C(CCO)C1]

In one embodiment, W is

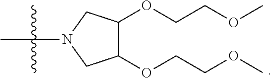 [SMILES: *N1CC(OCCOC)C(OCCOC)C1]

In one embodiment, W is

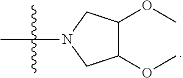 [SMILES: *N1CC(OC)C(OC)C1]

In one embodiment, W is

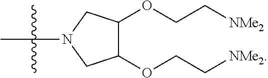 [SMILES: *N1CC(OCCN(C)C)C(OCCN(C)C)C1]

In one embodiment, W is

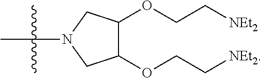

In one embodiment, W is

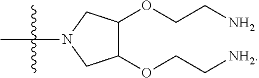 [SMILES: *N1CC(OCCN)C(OCCN)C1]

In some embodiments, W is

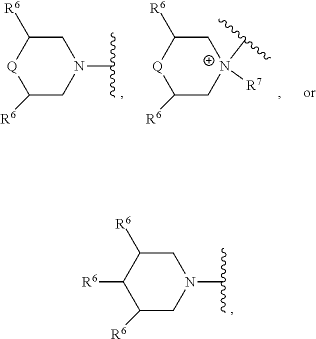

wherein each R⁶ is independently H, C₁-C₃ alkyl, hydroxyl, hydroxyalkyl, alkoxy, —O-alkylene-O-alkyl, or —O-alkylene-N(R⁷)₂; Q is —O—, —C(R⁷)₂—, or —N(R⁷); and R⁷ is H, C₁-C₃ alkyl, or hydroxyalkyl. In one embodiment, W is

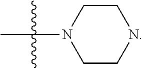

In one embodiment, W is

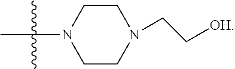 [SMILES: *N1CCN(CCO)CC1]

In one embodiment, W is

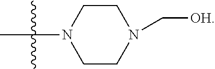 [SMILES: *N1CCN(CO)CC1]

In one embodiment, W is

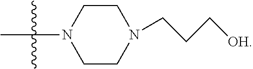 [SMILES: *N1CCN(CCCO)CC1]

In one embodiment W is

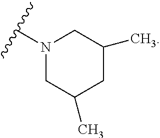 [SMILES: *N1CC(C)CC(C)C1]

In one embodiment, W is

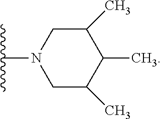 [SMILES: *N1CC(C)C(C)C(C)C1]

In one embodiment W is

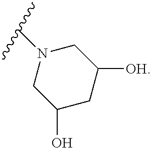 [SMILES: *N1CC(O)CC(O)C1]

In one embodiment, W is

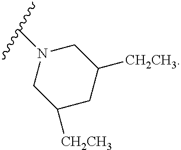 [SMILES: *N1CC(CC)CC(CC)C1]

In one embodiment, W is

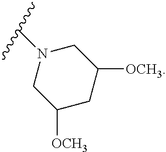 [SMILES: *N1CC(OC)CC(OC)C1]

In one embodiment, W is

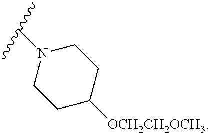

In one embodiment, W is

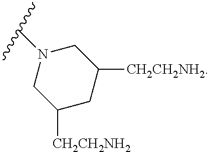

In one embodiment, W is

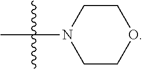 [SMILES: *N1CCOCC1]

In one embodiment, W is

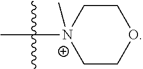 [SMILES: *[N+]1(C)CCOCC1]

In one embodiment, W is

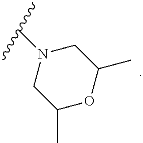 [SMILES: *N1CC(C)OC(C)C1]

In one embodiment, W is

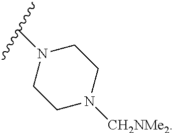

In one embodiment, W is

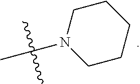 [SMILES: *N1CCCCC1]

In one embodiment, W is

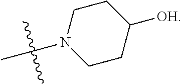 [SMILES: *N1CCC(O)CC1]

In one embodiment, W is

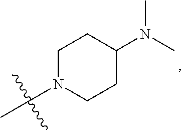 [SMILES: *N1CCC(N(C)C)CC1]

In some embodiments, W is

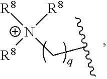

wherein q is 0, and each R⁸ is independently H, C₁-C₃ alkyl, or hydroxyalkyl. In one embodiment, W is

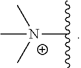 [SMILES: *[N+](C)(C)C]

In one embodiment, W is

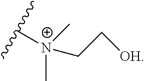 [SMILES: *[N+](C)(C)CCO]

In some embodiments, W is

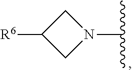

wherein R⁶ is independently H, alkyl, hydroxyl, hydroxyalkyl, alkoxy, —O-alkylene-O-alkyl, or —O-alkylene-N(R⁷)₂; and each R⁷ is independently H or C₁-C₃ alkyl. In one embodiment, W is

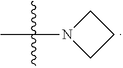 [SMILES: *N1CCC1]

In one embodiment, W is

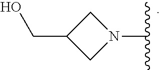 [SMILES: *N1CC(CO)C1]

In one embodiment, W is

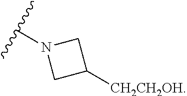

In one embodiment, W is

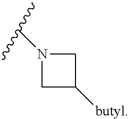

In one embodiment W is

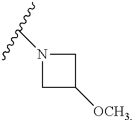 [SMILES: *N1CC(OC)C1]

In one embodiment, W is

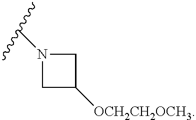

In some embodiments, W is

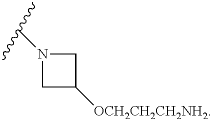

In some embodiments, W is

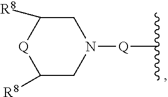

wherein each R⁸ is independently H, C₁-C₃ alkyl, or hydroxyalkyl; each Q is independently absent, —O—, —CO—, —C(R⁷)₂—, or —N(R⁷)—; and each R⁷ is independently H, C₁-C₃ alkyl, alkylamino, alkylaminoalkyl, or aminoalkyl. In one embodiment, W is

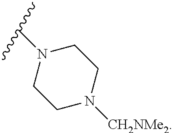

In one embodiment, W is

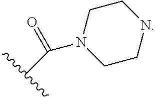

In some embodiments, W is

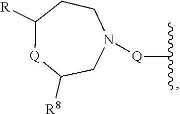

wherein each R⁸ is independently H, C₁-C₃ alkyl, or hydroxyalkyl; each Q is independently absent, —O—, —CO—, —C(R⁷)₂—, or —N(R⁷)—; and each R⁷ is independently H, C₁-C₃ alkyl, alkylamino, alkylaminoalkyl, or aminoalkyl. In one embodiment. W is

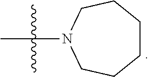 [SMILES: *N1CCCCCC1]

In one embodiment, W is

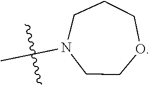 [SMILES: *N1CCCOCC1]

In some embodiments, provided herein is a lipid comprising at least one head group and at least one tail group, wherein:
the tail group has a structure of formula (TI) or (TI′)

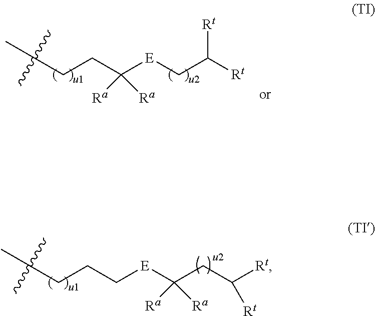

a pharmaceutically acceptable salt thereof, or a stereoisomer of any of the foregoing,
   wherein:
   E is each independently a biodegradable group;
   R^{a} is each independently C₁-C₅ alkyl, C₂-C₅ alkenyl, or C₂-C₅ alkynyl;
   u1 and u2 are each independently 0, 1, 2, 3, 4, 5, 6, or 7;
   R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
   [symbol] represents the bond connecting the tail group to the head group; and
the head group has a structure of one of the following formulas:
   i)

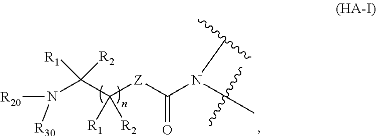

wherein:
   R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
   R₂₀ and R₃₀, together with the adjacent N atom, form a 3 to 7 membered heterocylic or heteroaromatic ring containing one or more heteroatoms, optionally substituted with one or more OH, SH, halogen, alkyl, or cycloalkyl groups;
   each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
   each of R₁₀ and R₁₁ is independently H, C₁-C₃ branched or unbranched alkyl, C₂-C₃ branched or unbranched alkenyl; or R₁₀ and R₁₁ together form a heterocyclic ring;
   n is 0, 1, 2, 3 or 4;
   Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, SH;
ii)

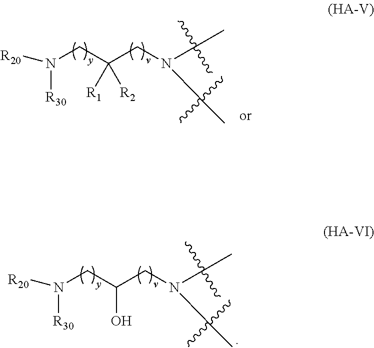

wherein:
   R₁ is H, C₁-C₃ alkyl, OH, halogen, SH, or NR₁₀R₁₁;
   R₂ is OH, halogen, SH, or NR₁₀R₁₁; or R₁ and R₂ can be taken together to form a cyclic ring;
   R₁₀ and R₁₁ are each independently H or C₁-C₃ alkyl; or R₁₀ and R₁₁ can be taken together to form a heterocyclic ring;
   R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
   each of v and y is independently 1, 2, 3, or 4;
iii)

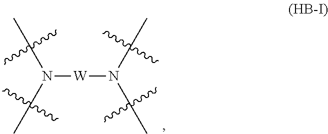

wherein W is

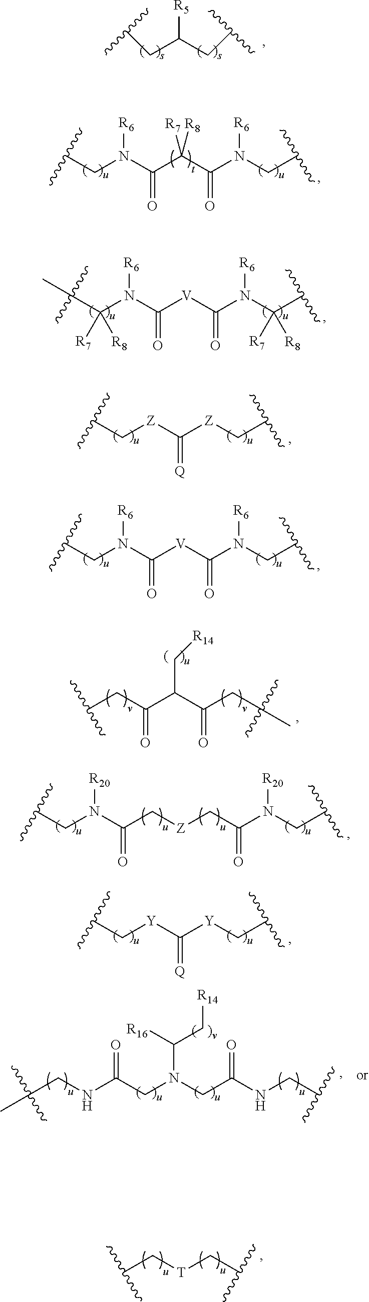

wherein
   R₅ is OH, SH(CH₂)ₛOH, or NR₁₀R₁₁;
   each R₆ is independently H, C₁-C₃ branched or unbranched alkyl, C₂-C₃ branched or unbranched alkenyl, or cycloalkyl;
   each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or R₇ and R₈ are taken together to form a ring;
   each R₂₀ is independently H, or C₁-C₃ branched or unbranched alkyl;
   R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
   R₁₆ is H, ═O, ═S, or CN;
   each of s, u, and t is independently 1, 2, 3, 4, or 5;
   each v is independently 0, 1, 2, 3, 4, or 5;
   each Y is a divalent heterocyclic;
   each Z is independently absent, O, S, or NR₁₂, wherein R₁₂ is H, C₁-C₇ branched or unbranched alkyl, or C₂-C₇ branched or unbranched alkenyl;
   Q is O, S, CH₂, or NR₁₃, wherein each R₁₃ is H, C₁-C₅ alkyl;
   V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
   T is —NHC(O)O—, —OC(O)NH—, or a divalent heterocyclic;
iv)

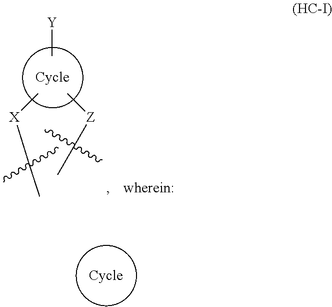

is cyclic or heterocyclic moiety;
Y is alkyl, hydroxy, hydroxyalkyl,

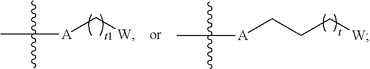

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, or —S—S—;
each of X and Z is independently absent, —O—, —C(O)—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
wherein the lipid has a pKa from about 4 to about 8.

In some embodiments, provided herein is a lipid comprising at least one head group and at least one tail group, wherein:
at least one tail group has the structure of at least one of the following formulas:

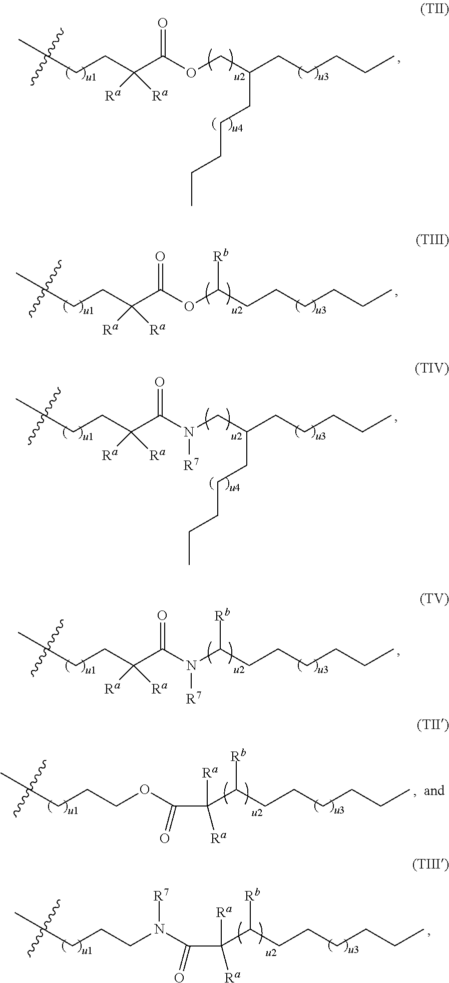

wherein:
R⁷ is each independently H or methyl;
R^{b} is in each occasion independently H or C₁-C₄ alkyl;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
u3 and u4 are each independently 0, 1, 2, 3, 4, 5, 6, or 7; and
the head group has a structure of one of the following formulas:

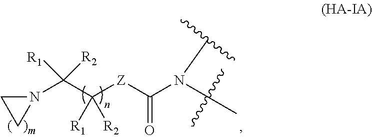

wherein m is 1, 2, 3, 4, 5, 6, 7 or 8;

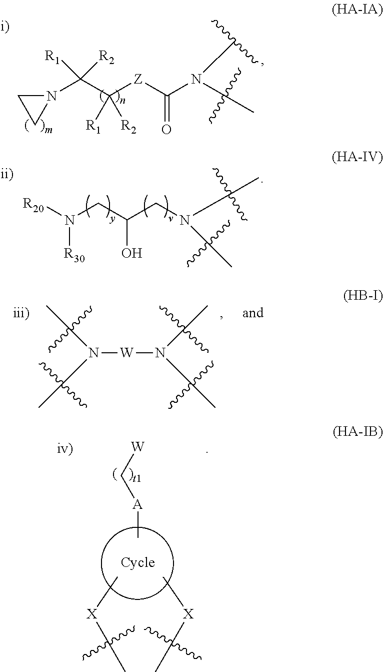

In some embodiments, in the above lipids, at least one tail group has the structure of formula (TII), (TIII), (TIV), (TV), (TII′), or (TIII′), wherein each R^{a} is methyl; u1 is 3-5, u2 is 0-3; and u3 and u4 are each independently 1-7.

In some embodiments, in the above lipids, the head group has the structure of one of the following formulas:

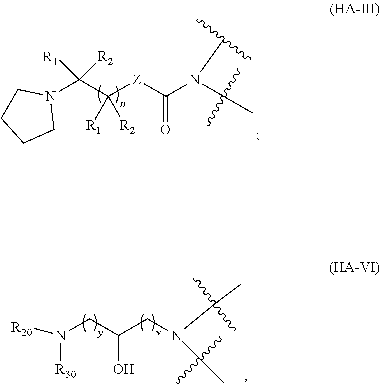

wherein each R₂₀ and R₃₀ are independently C₁-C₃ alkyl.

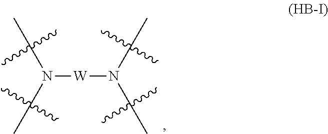

wherein:
   W is

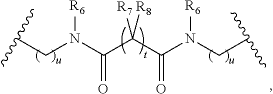

wherein:
   each R₆, R₇, and R₈ are independently H or methyl; and
   each of u and t is independently 1, 2, or 3; or
   W is

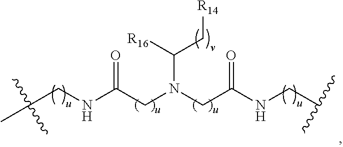

wherein:
   R₁₆ is H or ═O;
   R₁₄ is a nitrogen-containing 5- or 6-membered heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H or C₁-C₃ alkyl; and
   each of u and v is independently 1, 2, or 3; or
W is

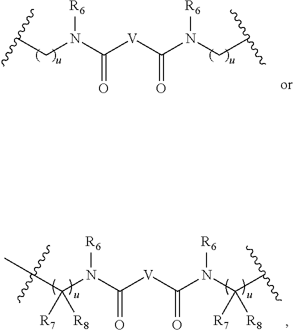

wherein:
   each R₆ is independently H or methyl;
   each R₇ is independently H;
   each R₈ is methyl;
   each u is independently 1, 2, or 3; and
   V is C₂-C₆ alkylene or C₂-C₆ alkenylene or
   W is

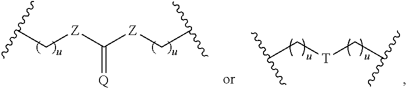

wherein:
   each u is independently 1, 2, or 3;
   each Z is independently NR₁₂; and
   T is a divalent nitrogen-containing 5- or 6-membered heterocyclic;
and

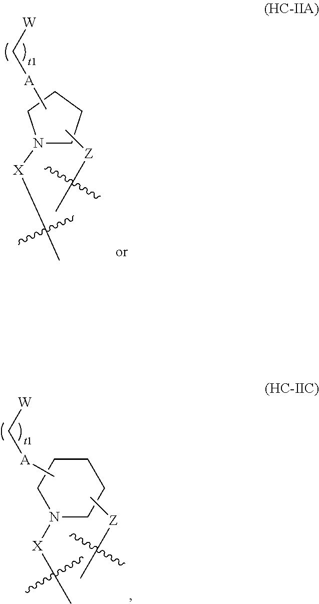

wherein:
W is hydroxyl, substituted or unsubstituted hydroxyalkyl, one of the following moieties:

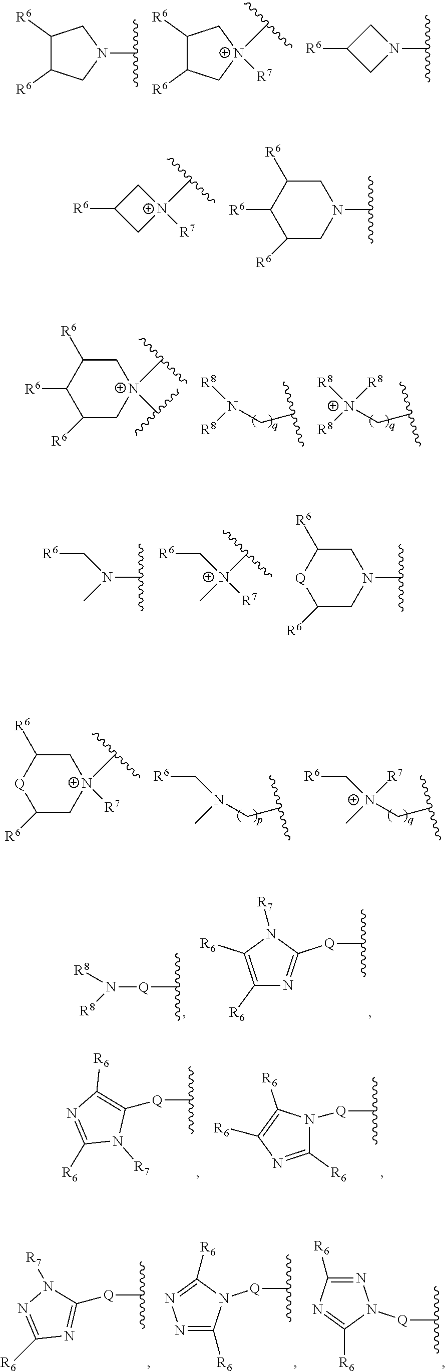

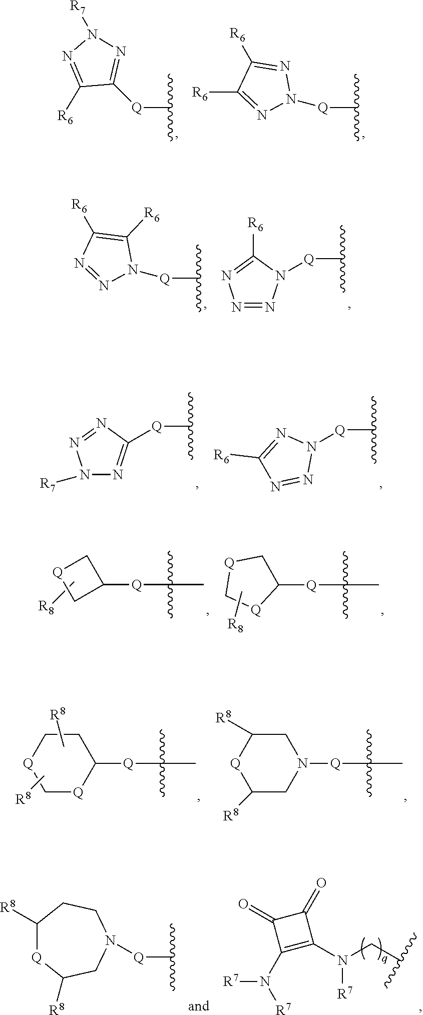

wherein
   each Q is independently absent, —O—, —C(O)—, —C(S)—, —C(O)O—, —(CH₂)_{q}—C(R⁷)₂—, —C(O)N(R⁷)—, —C(S)N(R⁷)—, or —N(R⁷);
   R⁶ is independently H, alkyl, hydroxyl, hydroxyalkyl, alkoxy, —O-alkylene-O-alkyl, —O-alkylene-N(R⁷)₂, amino, alkylamino, aminoalkyl, thiol, thiolalkyl, or N⁺(R⁷)₃-alkylene-Q-;
   each R⁸ is independently H, alkyl, hydroxyalkyl, amino, aminoalkyl, alkylamino, thiol, thiolalkyl, heterocyclyl, heteroaryl; or two R⁸ together with the nitrogen atom form a ring, optionally substituted with one or more alkyl, hydroxy, hydroxyalkyl, alkoxy, alkylaminoalkyl, alkylamino, or aminoalkyl;
   q is 0, 1, 2, 3, 4, or 5; and
   p is 0, 1, 2, 3, 4, or 5.

Another aspect of the invention relates to a lipid comprising at least two lipophilic tail groups, and a head group of formula (G-HC-IIID):

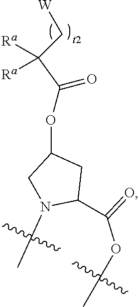

a pharmaceutically acceptable salt thereof, or a stereoisomer of any of the foregoing,
wherein:
R^{a} is each independently C₁-C₅alkyl, C₂-C₅ alkenyl, or C₂-C₅ alkynyl;
t2 is an integer from 0 to 5;
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
[symbol] represents the bond connecting the head group to the tail groups.

In some embodiments, each R^{a} is methyl, and t2 is 0-3.

In some embodiments, W is hydroxyl, substituted or unsubstituted hydroxyalkyl, or one of the following moieties:

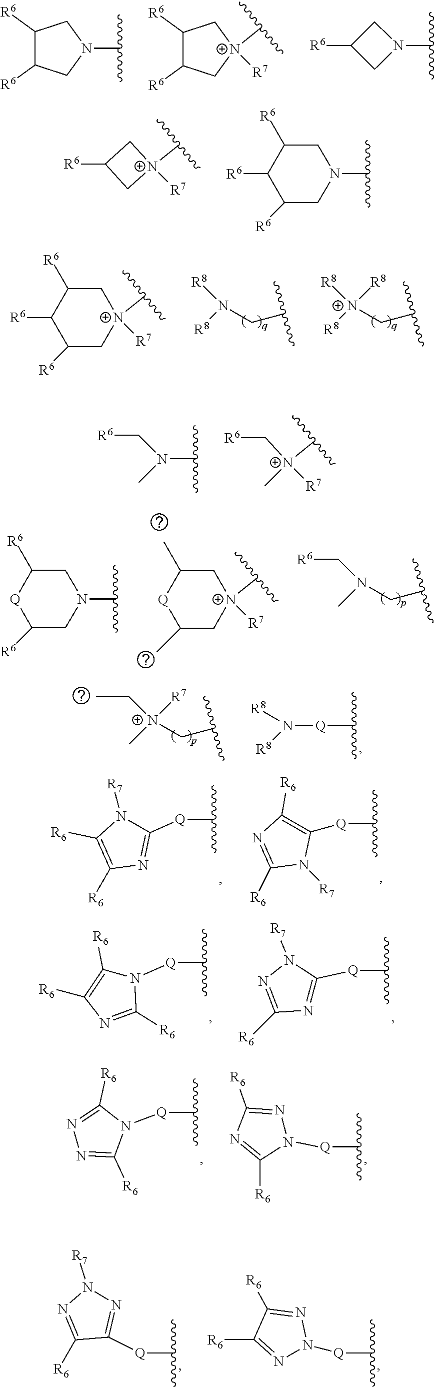

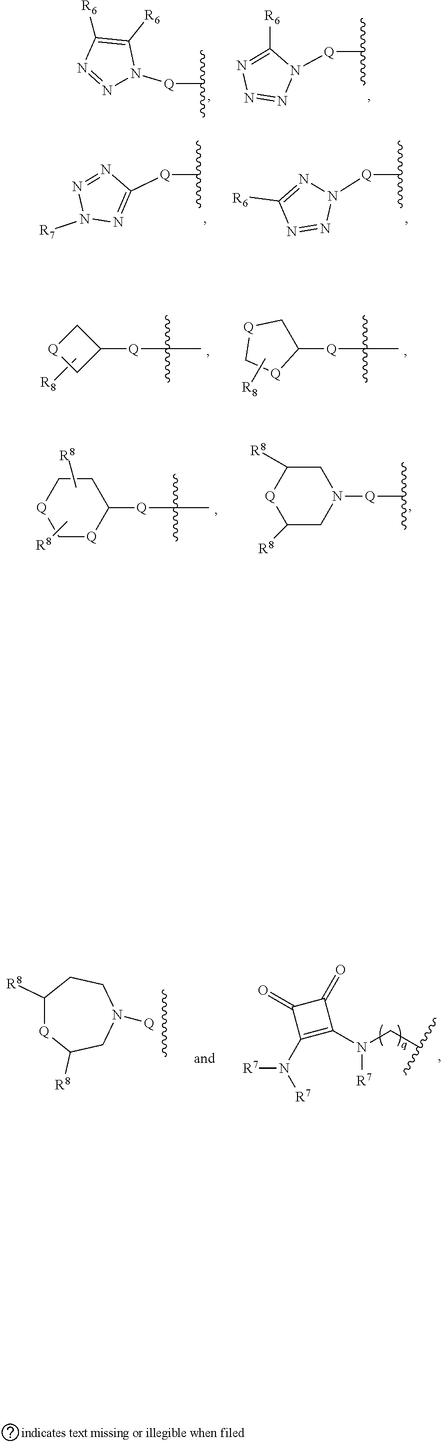

wherein
each Q is independently absent, —O—, —C(O)—, —C(S)—, —C(O)O—, —(CH₂)_{q}—C(R⁷)₂—, —C(O)N(R⁷)—, —C(S)N(R⁷)—, or —N(R⁷);
R⁶ is independently H, alkyl, hydroxyl, hydroxyalkyl, alkoxy, —O-alkylene-O-alkyl, —O-alkylene-N(R⁷)₂, amino, alkylamino, aminoalkyl, thiol, thiolalkyl, or N⁺(R⁷)₃-alkylene-Q-;
each R⁸ is independently H, alkyl, hydroxyalkyl, amino, aminoalkyl, alkylamino, thiol, thiolalkyl, heterocyclyl, heteroaryl; or two R⁸ together with the nitrogen atom form a ring, optionally substituted with one or more alkyl, hydroxy, hydroxyalkyl, alkoxy, alkylaminoalkyl, alkylamino, or aminoalkyl;
q is 0, 1, 2, 3, 4, or 5; and
p is 0, 1, 2, 3, 4, or 5.

In some embodiments, W is

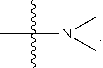 [SMILES: *N(C)C]

In one embodiment, the lipid has the structure:

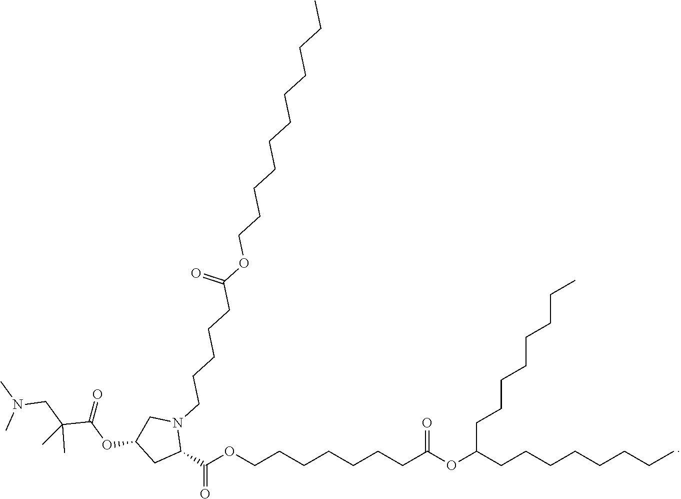 [SMILES: CCCCCCCCCCCOC(=O)CCCCCN1C[C@@H](OC(=O)C(C)(C)CN(C)C)C[C@H]1C(=O)OCCCCCCCC(=O)OC(CCCCCCCC)CCCCCCCC]

Also disclosed herein are a nucleic acid-lipid particle comprising:
a nucleic acid;
one or more lipid compounds comprising at least one head group (e.g., HA-I to HA-VII, HB-I, or HC-I to HC-IIIE; or any subgenus or species of these formulas disclosed herein), and at least one tail group of formula (T to TIII, or TI′ to TIII′, or any subgenus or species of these formulas disclosed herein);
a helper lipid;
a sterol; and
a PEG-modified lipid.

Also disclosed herein are a pharmaceutical composition comprising a lipid particle and a pharmaceutically acceptable diluent, wherein the lipid particle comprises:
a nucleic acid;
35-65 mol % of one or more lipid compounds comprising at least one head group (e.g., HA-I to HA-VII, HB-I, or HC-I to HC-IIIE; or any subgenus or species of these formulas disclosed herein), and at least one tail group of formula (T to TIII, or TI′ to TIII′, or any subgenus or species of these formulas disclosed herein);
3-12 mol % of a helper lipid
15-45 mol % of a steorol; and
0.5-10 mol % of a PEG-modified lipid.

Also disclosed herein are pharmaceutical compositions comprising one or more lipid compounds comprising at least one head group (e.g., HA-I to HA-VII, HB-I, or HC-I to HC-IIIE; or any subgenus or species of these formulas disclosed herein), and at least one tail group of formula (T to TIII, or TI′ to TIII′, or any subgenus or species of these formulas disclosed herein) and a therapeutic agent. In some embodiments, the pharmaceutical compositions further comprise one or more components selected from neutral lipids, charged lipids, steroids, and polymer conjugated lipids. Such compositions may be useful for formation of lipid nanoparticles for delivery of a therapeutic agent.

In some embodiments, the present disclosure provides methods for delivering a therapeutic agent to a patient in need thereof, comprising administering to said patient a lipid nanoparticle composition comprising one or more lipid compounds comprising at least one head group (e.g., HA-I to HA-VII, HB-I, or HC-I to HC-IIIE; or any subgenus or species of these formulas disclosed herein), and at least one tail group of formula (T to TIII, or TI′ to TIII′, or any subgenus or species of these formulas disclosed herein), a pharmaceutically acceptable salt thereof, and/or a stereoisomer of any of the foregoing and the therapeutic agent. In some embodiments, the method further comprises preparing a lipid nanoparticle composition comprising one or more lipid compounds comprising at least one head group (e.g., HA-I to HA-VII, HB-I, or HC-I to HC-IIIE; or any subgenus or species of these formulas disclosed herein), and at least one tail group of formula (T to TIII, or TI′ to TIII′, or any subgenus or species of these formulas disclosed herein), a pharmaceutically acceptable salt thereof, and/or a stereoisomer of any of the foregoing and a therapeutic agent.

In some embodiments, the total therapeutic cargo administered to the subject has a spleen to liver ratio of at least 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, or 10. In some embodiments, the total therapeutic cargo administered to the subject has a spleen to liver ratio of at least 1. In some embodiments, the total therapeutic cargo administered to the subject has spleen to liver ratio of at least 5.

These and other aspects of the disclosure will be apparent upon reference to the following detailed description.

### DETAILED DESCRIPTION OF INVENTION

### Definitions

As used herein, the following terms have the meanings ascribed to them unless specified otherwise.

Unless defined otherwise, all technical and scientific terms used herein have the same meaning as is commonly understood by one of skill in the art to which this disclosure belongs.

As used in the specification and claims, the singular form “a” “an” and “the” include plural references unless the context clearly dictates otherwise.

Unless the context requires otherwise, throughout the present specification and claims, the word “comprise” and variations thereof, such as, “comprises” and “comprising” are to be construed in an open and inclusive sense, that is, as “including, but not limited to”.

The phrase “induce expression of a desired protein” refers to the ability of a nucleic acid to increase expression of the desired protein. To examine the extent of protein expression, a test sample (e.g., a sample of cells in culture expressing the desired protein) or a test mammal (e.g. a mammal such as a human or an animal) model such as a rodent (e.g., mouse) or a non-human primate (e.g., monkey) model is contacted with a nucleic acid (e.g., nucleic acid in combination with a lipid of the present disclosure). Expression of the desired protein in the test sample or test animal is compared to expression of the desired protein in a control sample (e.g., a sample of cells in culture expressing the desired protein) or a control mammal (e.g., a mammal such as a human or an animal) model such as a rodent (e.g., mouse) or non-human primate (e.g., monkey) model that is not contacted with or administered the nucleic acid. When the desired protein is present in a control sample or a control mammal, the expression of a desired protein in a control sample or a control mammal may be assigned a value of 1.0. In some embodiments, inducing expression of a desired protein is achieved when the ratio of desired protein expression in the test sample or the test mammal to the level of desired protein expression in the control sample or the control mammal is greater than 1, for example, about 1.1, 1.5, 2.0, 5.0 or 10.0. When a desired protein is not present in a control sample or a control mammal, inducing expression of a desired protein is achieved when any measurable level of the desired protein in the test sample or the test mammal is detected. One of ordinary skill in the art will understand appropriate assays to determine the level of protein expression in a sample, for example dot blots, northern blots, in situ hybridization, ELISA, immunoprecipitation, enzyme function, and phenotypic assays, or assays based on reporter proteins that can produce fluorescence or luminescence under appropriate conditions.

The phrase “inhibiting expression of a target gene” refers to the ability of a nucleic acid to silence, reduce, or inhibit the expression of a target gene. To examine the extent of gene silencing, a test sample (e.g., a sample of cells in culture expressing the target gene) or a test mammal (e.g., a mammal such as a human or an animal) model such as a rodent (e.g., mouse) or a non-human primate (e.g., monkey) model is contacted with a nucleic acid that silences, reduces, or inhibits expression of the target gene. Expression of the target gene in the test sample or test animal is compared to expression of the target gene in a control sample (e.g., a sample of cells in culture expressing the target gene) or a control mammal (e.g., a mammal such as a human or an animal) model such as a rodent (e.g., mouse) or non-human primate (e.g., monkey) model that is not contacted with or administered the nucleic acid. The expression of the target gene in a control sample or a control mammal may be assigned a value of 100%. In some embodiments, silencing, inhibition, or reduction of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0% In other words, the nucleic acids are capable of silencing, reducing, or inhibiting the expression of a target gene by at least about 5%, 10%, 15%, 20%, 25%, 30%, 35%, 40%, 45%, 50%, 55%, 60%, 65%, 70%, 75%, 80%, 85%, 90%, 95%, or 100% in a test sample or a test mammal relative to the level of target gene expression in a control sample or a control mammal not contacted with or administered the nucleic acid. Suitable assays for determining the level of target gene expression include, without limitation, examination of protein or mRNA levels using techniques known to those of skill in the art, such as, e.g., dot blots, northern blots, in situ hybridization, ELISA, immunoprecipitation, enzyme function, as well as phenotypic assays known to those of skill in the art.

An “effective amount” or “therapeutically effective amount” of an active agent or therapeutic agent such as a therapeutic nucleic acid is an amount sufficient to produce the desired effect, e.g., an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as mRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%), 15%), 10%), 5%), or 0% Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2-0-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “lipids” refers to a group of organic compounds that include, but are not limited to, esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

A “steroid” is a compound comprising the following carbon skeleton:

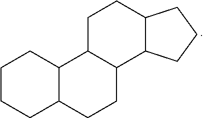 [SMILES: C1CCC2C(C1)CCC1C3CCCC3CCC21]

A non-limiting example of a steroid is cholesterol.

As used herein, the term “compound,” is meant to include all the isomers and isotopes of the structure depicted, all the pharmaceutically acceptable salts, solvates, or hydrates thereof, and all crystal forms (e.g., crystal polymorphs), crystal form mixtures, or anhydrides or hydrates thereof.

“Isotopes” refers to atoms having the same atomic number but different mass numbers resulting from a different number of neutrons in the nuclei. For example, isotopes of hydrogen include tritium (3H) and deuterium (2H).

“Isomers.” The compounds described herein or their pharmaceutically acceptable salts may include all isomers, such as geometrical isomers, optical isomers based on an asymmetrical carbon, stereoisomers, tautomers, and the like. For instance, the compounds can contain one or more stereocenters and may thus give rise to geometric isomers (e.g., double bond causing geometric E/Z isomers), enantiomers, diastereomers (e.g., enantiomers (i.e., (+) or (−)) or cis/trans isomers), and other stereoisomeric configurations that may be defined, in terms of absolute stereochemistry, as (R)- or (S)- such as for sugar anomers, or as (D)- or (L)- such as for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Enantiomeric and stereomeric mixtures of compounds and means of resolving them into their component enantiomers or stereoisomers are well-known. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility.

Recrystallization solvent, rate of crystallization, storage temperature, and other factors may cause one crystal form to dominate. Crystal polymorphs of the compounds can be prepared by crystallization under different conditions. Crystallization of the compounds disclosed herein may produce a solvate.

As used herein, the term “solvate” refers to an aggregate that comprises one or more molecules of an ionizable lipid of the disclosure with one or more molecules of solvent. The solvent may be water, in which case the solvate may be a hydrate, including a monohydrate, dihydrate, hemihydrate, sesquihydrate, trihydrate, tetrahydrate and the like. Alternatively, the solvent may be an organic solvent.

As used herein, “ionizable lipid” refers to a lipid capable of being charged. In some embodiments, an ionizable lipid includes one or more positively charged amine groups. In some embodiments, ionizable lipids are ionizable such that they can exist in a positively charged or neutral form depending on pH. The ionization of an ionizable lipid affects the surface charge of a lipid nanoparticle comprising the ionizable lipid under different pH conditions. The surface charge of the lipid nanoparticle in turn can influence its plasma protein absorption, blood clearance, and tissue distribution (Semple, S. C., et al., Adv. Drug Deliv Rev 32:3-17 (1998)) as well as its ability to form endosomolytic non-bilayer structures (Hafez, I. M., et al., Gene Ther 8: 1188-1196 (2001)) that can influence the intracellular delivery of nucleic acids.

The term “polymer conjugated lipid” refers to a molecule comprising both a lipid portion and a polymer portion. A non-limiting example of a polymer conjugated lipid is a pegylated lipid. The term “pegylated lipid” refers to a molecule comprising both a lipid portion and a polyethylene glycol portion. Pegylated lipids are known in the art and include, for example, 1-(monomethoxy-polyethyleneglycol)-2,3-dimyristoylglycerol (PEG-DMG) and the like. As used herein, the terms “PEG-lipid” and “PEGylated lipid” are interchangeable and refer to a lipid comprising a polyethylene glycol component.

The term “neutral lipid” refers to any of a lipid that exists either in an uncharged or neutral zwitterionic form at a selected pH. At physiological pH, such lipids include, but are not limited to, phosphotidylcholines such as 1,2-distearoyl-sn-glycero-3-phosphocholine (DSPC), 1,2-dipalmitoyl-5n-glycero-3-phosphocholine (DPPC), 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), sphingomyelins (SM), ceramides, and steroids such as sterols and their derivatives. Neutral lipids may be synthetic or naturally derived.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

The term “liposome” as used herein refers to a composition comprising an outer lipid layer membrane (e.g., a single lipid bi-layer known as unilamellar liposomes or multiple lipid bilayers known as multilamellar liposomes) surrounding an internal aqueous space which may contain a cargo. See, e.g., Cullis et ah, Biochim. Biophys Acta, 559:399-420 (1987), which is incorporated herein by reference in its entirety. A unilamellar liposome generally has a diameter in the range of about 20 to about 400 nanometers (nm), about 50 to about 300 nm, about 100 to about 200 nm, or about 300 to about 400 nm. A multilamellar liposome usually has a diameter in the range of about 1 to about 10 μm and may comprise anywhere from 2 to hundreds of concentric lipid bilayers alternating with layers of an aqueous phase.

The term “lipid nanoparticle” refers to a particle having at least one dimension on the order of nanometers (e.g., 1-1,000 nm) and comprising one or more compound of Formula (I). In some embodiments, lipid nanoparticles comprising one or more compounds of Formula (I), pharmaceutically acceptable salts thereof, and/or stereoisomers of any of the foregoing, are included in a composition that can be used to deliver a therapeutic agent, such as a nucleic acid (e.g., mRNA), to a target site of interest (e.g., cell, tissue, organ, tumor, and the like). In some embodiments, lipid nanoparticles comprise one or more compounds of Formula (I), pharmaceutically acceptable salts thereof, and/or stereoisomers of any of the foregoing, and a nucleic acid. In some embodiments, lipid nanoparticles comprise one or more compounds of Formula (I), pharmaceutically acceptable salts thereof, and/or stereoisomers of any of the foregoing, and a nucleic acid, and one or more other lipids selected from neutral lipids, charged lipids, steroids, and polymer conjugated lipids. In some embodiments, the therapeutic agent, such as a nucleic acid, may be encapsulated in a lipid portion of the lipid nanoparticle or an aqueous space enveloped by some or all of a lipid portion of the lipid nanoparticle, thereby protecting it from enzymatic degradation or other undesirable effects induced by the mechanisms of the host organism or cells, e.g., an adverse immune response.

In some embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In some embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures of which are herein incorporated by reference in their entirety for all purposes.

As used herein, the term “size” refers to the hydrodynamic diameter of a lipid nanoparticle population. The measurement of the size of a lipid nanoformulation may be used to indicate the size and population distribution (polydispersity index, PDI) of the composition.

As used herein, the “polydispersity index” is a ratio between weight-average molar mass and Mn is the number-average molar mass that describes the homogeneity of the particle size distribution of a system. A small value, e.g., less than 0.3, indicates a narrow particle size distribution.

A poly dispersity index may be used to indicate the homogeneity of a lipid composition (e.g., liposome or LNP), e.g., the particle size distribution of the liposome or LNP. A small (e.g., less than 0.3) polydispersity index generally indicates a narrow particle size distribution. A lipid composition may have a polydispersity index from about 0 to about 0.25, such as 0.01, 0.02, 0.03, 0.04, 0.05, 0.06, 0.07, 0.08, 0.09, 0.10, 0.11, 0, 12, 0.13, 0.14, 0.15, 0.16, 0.17, 0.18, 0.19, 0.20, 0.21, 0.22, 0.23, 0.24, or 0.25. In some embodiments, the polydispersity index of the lipid composition may be from about 0.10 to about 0.20.

As used herein, the term “apparent pKa” refers to the pH at which 50% of the lipid nanoformulation (e.g., LNP) is protonated. This can be used as an indicator of the pH range that the lipid nanoformulation (e.g., LNP) will be protonated, and thus initiate the endosomal escape process in a nucleotide delivery.

As used herein, the term “zeta potential” refers to the electrokinetic potential of lipid, e.g., in a lipid nanoformulation (e.g., a LNP composition). The zeta potential may describe the surface charge of a LNP composition. Zeta potential is useful in predicting organ tropism and potential interaction with serum proteins.

The zeta potential of a lipid composition (e.g., liposome or LNP) may be used to indicate the electrokinetic potential of the composition. In some embodiments, the zeta potential may describe the surface charge of a liposome or LNP. Lipid compositions (e.g., liposomes or LNP) with relatively low charges, positive or negative, are generally desirable, as more highly charged species may interact undesirably with cells, tissues, and other elements in the body. In some embodiments, the zeta potential of a liposome or LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

As used herein. “encapsulated” by a lipid refers a therapeutic agent, such as a nucleic acid (e.g., mRNA), that is fully or partially encapsulated to by lipid nanoparticle. In some embodiments, nucleic acid (e.g., mRNA) is fully encapsulated in a lipid nanoparticle.

As used herein. “encapsulation efficiency” or “entrapment efficiency” refers to the percentage of an encapsulated cargo (e.g., a therapeutic and/or prophylactic agent) that is successfully incorporated into (e.g., encapsulated or otherwise associated with) the lipid composition (e.g., a LNP or liposome), relative to the initial total amount of therapeutic and/or prophylactic agent provided. For example, if 97 mg of therapeutic and/or prophylactic agent are encapsulated in a lipid composition out of a total 100 mg of therapeutic and/or prophylactic agent initially provided, the encapsulation efficiency may be given as 97%. Encapsulation efficiency can be used to indicate the efficiency of an encapsulated cargo (e.g., a nucleic acid molecule) loading into the lipid composition using a particular formulation method and formulation recipe.

The efficiency of encapsulation of a cargo such as a protein and/or nucleic acid, describes the amount of protein and/or nucleic acid that is encapsulated or otherwise associated with a lipid composition (e.g., liposome or LNP) after preparation, relative to the initial amount provided. The encapsulation efficiency is desirably high (e.g., at least 70%, 80%, 90%, 95%, close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the liposome or LNP described herein, the encapsulation efficiency of a protein and/or nucleic acid may be at least 50%, for example 50%, 55%, 60%, 65%, 70%, 75%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

“Serum-stable” in relation to nucleic acid-lipid nanoparticles means that the nucleic acid is not significantly degraded after exposure to a serum or nuclease assay that would significantly degrade free DNA or RNA. Suitable assays include, for example, a standard serum assay, a DNAse assay, or an RNAse assay.

Some techniques of administration can lead to systemic delivery of certain agents but not others. “Systemic delivery” means that a useful, such as a therapeutic, amount of an agent is delivered to most parts of the body. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery.

“Local delivery,” as used herein, refers to delivery of an agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous or intradermal injection. Local delivery does not preclude a systemic pharmacological effect.

As used herein, “methods of administration” may include both systemic delivery and local delivery. “Systemic delivery” means that a useful, such as a therapeutic, amount of an agent is delivered to most parts of the body. Systemic delivery of a liposome or LNP can be carried out by any means known in the art including, for example, intravenous, intraarterial, intramuscular, intradermal, subcutaneous, and intraperitoneal delivery. In some embodiments, systemic delivery of lipid nanoparticles is by intravenous delivery. “Local delivery,” as used herein, refers to delivery of an agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous or intradermal injection. Local delivery does not preclude a systemic pharmacological effect. As used herein, the term “polypeptide” or “polypeptide of interest” refers to a polymer of amino acid residues typically joined by peptide bonds that can be produced naturally (e.g., isolated or purified) or synthetically.

“Nucleic acid” is meant to define an oligonucleotide or polynucleotide sequence. Non-limiting examples of oligonucleotide or polynucleotides are DNA, plasmid DNA, self-amplifying RNA, mRNA, siRNA and tRNA. The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a poly A sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, and mixtures thereof.

“Alkyl” refers to a straight or branched hydrocarbon chain radical consisting solely of carbon and hydrogen atoms, having, for example, from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈alkyl) or one to six carbon atoms (C₁-C₆ alkyl) and which is attached to the rest of the molecule by a single bond, e.g., methyl, ethyl, n-propyl, 1-methylethyl (isopropyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group is optionally substituted.

“Alkylene” or “alkylene chain” refers to a straight or branched divalent hydrocarbon chain linking the rest of the molecule to a radical group, consisting solely of carbon and hydrogen, having, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain.

The term “alkenyl” refers to a straight or branched hydrocarbon chain having one or more double bonds. Unless otherwise indicated. “alkenyl” generally refers to C₂-C₈ alkenyl (e.g., C₂-C₆ alkenyl, C₂-C₄ alkenyl, or C₂-C₃ alkenyl). Examples of a typical alkenyl include, but not limited to, allyl, propenyl, 2-butenyl, 3-hexenyl and 3-octenyl groups. The term “alkynyl” refers to a straight or branched hydrocarbon chain containing 2-8 carbon atoms and characterized in having one or more triple bonds. Unless otherwise indicated, “alkynyl” generally refers to C₂-C₈ alkynyl (e.g., C₂-C₆ alkynyl, C₂-C₄ alkynyl, or C₂-C₃ alkynyl). Some examples of a typical alkynyl are ethynyl, 2-propynyl, and 3-methylbutynyl, and propargyl. The sp² and sp³ carbons may optionally serve as the point of attachment of the alkenyl and alkynyl groups, respectively.

The term “cycloalkyl” or “cyclyl” as employed herein includes saturated and partially unsaturated, but not aromatic, cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” or “heteroar-” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. The term also include groups in which a heteroaromatic ring is fused to one or more aryl, cycloalkyl, or heterocyclyl rings, where the radical or point of attachment is on the heteroaromatic ring. Examples of heteroaryl groups include pyrrolyl, pyridyl, pyridazinyl, pyrazolyl, triazolyl, tetrazolyl, oxazolyl, isoxazolyl, oxadiazolyl, furanyl, imidazolyl, benzimidazolyl, pyrimidinyl, pyrazinyl, indolizinyl, thiophenyl or thienyl, quinolinyl, indolyl, thiazolyl, isothiazolyl, thiadiazolyl, purinyl, naphthyridinyl, pteridinyl, isoindolyl, benzothienyl, benzofuranyl, dibenzofuranyl, indazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one and the like.

The term “heterocyclyl,” “heterocycle,” “heterocyclic radical,” or “heterocyclic ring” refers to a 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. As used herein it can generally include both nonaromatic or aromatic ring (e.g., generally covered by heteroaryl). The term also include groups in which a heterocycle ring is fused to one or more aryl, cycloalkyl, or heterocyclyl rings. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

Examples of heterocyclyl groups include trizolyl, tetrazolyl, piperazinyl, pyrrolidinyl, dioxanyl, dioxolanyl, diazepinyl, oxazepinyl, thiazepinyl, morpholinyl, tetrahydrofuranyl, tetrahydropyranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, quinuclidinyl, and the like.

Examples of heterocyclyl groups also include those typical heteroaryl groups such as pyrrolyl, pyridyl, pyridazinyl, pyrazolyl, triazolyl, tetrazolyl, oxazolyl, isoxazolyl, oxadiazolyl, furanyl, imidazolyl, benzimidazolyl, pyrimidinyl, pyrazinyl, indolizinyl, thiophenyl or thienyl, quinolinyl, indolyl, thiazolyl, isothiazolyl, thiadiazolyl, purinyl, naphthyridinyl, pteridinyl, isoindolyl, benzothienyl, benzofuranyl, dibenzofuranyl, indazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one and the like.

A divalent radical of an alkyl, alkenyl, aryl, heteroaryl, cycloalkyl, heterocyclyl is formed by removal of a hydrogen atom from an alkyl, alkenyl, aryl, heteroaryl, cycloalkyl, and heterocyclyl radical, respectively (or by removal of two hydrogen atoms from an alkane, alkene, arene, heteroarene, cycloalkane, or heterocycle, respectively).

The term “alkoxy” refers to an —O-alkyl radical.

The term “aminoalkyl” refers to an alkyl substituted with an amino. The term “alkylamino” refers to an amino substituted with an alkyl.

The term “aminocarbonyl” refers to an —C(O)-amino radical.

The term “substituted” used herein means any of the above groups (e.g., alkyl, hydroxyalkyl, alkylene, cycloalkyl, cycloalkylene, amino, aminocarbonyl, heterocyclyl, or heteroaryl) wherein one or more hydrogen atom is replaced by a bond to a non-hydrogen atom such as, but not limited to: a halogen atom such as F, CI, Br, or I; oxo groups (═O); hydroxyl groups (—OH); alkoxy, alkoxyalkyl, aralkoxy, alkyl such as C₁-C₁₂ alkyl groups; cycloalkyl groups; alkenyl, alkynyl, aryl, aralkyl heterocyclyl, heterocyclyl, heteroaryl, thiol, alkylthio, arylthio, alkylthioalkyl, arylthioalkyl, alkylsulfonyl, alkylsulfonylalkyl, arylsulfonylalkyl, aryloxy, carboxyalkyl, alkoxycarbonylalkyl, aminocarbonylalkyl, acyl, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, aralkoxycarbonyl, sulfonyl, alkylaminolactams, alkylaminoheteroaryls, alkylaminoheterocycyls, and aminosulfonamides. Exemplary substituents also include: —(C═O)OR; —O(C═O)R; —C(═O)R; —OR; —S(O)ₓR; —S—SR; —C(═O)SR; —SC(═O)R; —NRR′; —R′C(═O)R; —C(═O)RR′; —RC(═O)R′R″; —OC(═O)RR′; —RC(═O)OR′; —R′S(O)ₓR″R; —R′S(O)ₓR; and —S(O)ₓRR′, wherein: R, R′, and R″ is, at each occurrence, independently H, C₁-C₁₅ alkyl or cycloalkyl, heterocyclyl, or hereoaryl that can be optionally substituted, and x is 0, 1 or 2. In some embodiments, the substituent is a C₁-C₁₂ alkyl group. In some embodiments, the substituent is a cycloalkyl group. In some embodiments, the substituent is a halo group, such as fluoro. In some embodiments, the substituent is an oxo group. In some embodiments, the substituent is a hydroxyl group. In some embodiments, the substituent is a hydroxyalkylene group (—R—OH). In some embodiments, the substituent is an alkoxy group (—OR). In some embodiments, the substituent is a carboxyl group. In some embodiments, the substituent is an amine group (—NRR′). Suitable substituents also include divalent substituents on a saturated carbon atom, including but are not limited to: ═O, ═S, ═NNR*2, ═NNHC(O)R*, ═NNHC(O)OR*, ═NNHS(O)2R*, ═NR*, ═NOR*, —O(C(R*2))2-3O—, or —S(C(R*2))2-3S—, wherein each independent occurrence of R* is selected from hydrogen, substituted or unsubstituted C1-6 alkyl, or an unsubstituted 5-6-membered saturated or partially unsaturated ring, or an aryl ring having 0-4 heteroatoms independently selected from nitrogen, oxygen, or sulfur.

“Halo” or “halogen” refers to any radical of fluorine, chlorine, bromine or iodine.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

The present disclosure is also meant to encompass all pharmaceutically acceptable compounds of all the Formulas identified herein being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ²²P, ³⁵S, ¹⁸F, ³⁶Cl, ¹²³I, and ¹²⁵I, respectively. These isotopically-labelled compounds could be useful to help determine or measure the effectiveness of the compounds, by characterizing, for example, the site or mode of action, or binding affinity to pharmacologically important site of action. Certain isotopically-labelled compounds are useful in drug and/or substrate tissue distribution studies. The radioactive isotopes tritium, i.e., ³H, and carbon-14, i.e., ¹⁴C, may be useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be useful in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can generally be prepared by conventional techniques known to those skilled in the art or by processes analogous to those described in the Preparations and Examples as set out below using an appropriate isotopically-labeled reagent in place of the non-labeled reagent previously employed.

The present disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments of the disclosure include compounds produced by a process comprising administering an ionizable lipid of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts. “Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, sodium, potassium, lithium, ammonium, calcium, magnesium, iron, zinc, copper, manganese, aluminum salts and the like. Non-limiting examples of inorganic salts are ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Non-limiting examples of organic bases are isopropylamine, diethylamine, ethanolamine, trimethylamine, dicyclohexylamine, choline and caffeine.

Crystallization of ionizable lipid(s) disclosed herein may produce a solvate. As used herein, the term “solvate” refers to an aggregate that comprises one or more molecules of an ionizable lipid of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. Solvates of compound of the disclosure may be true solvates, while in other cases, the compound of the disclosure may merely retain adventitious water or be a mixture of water plus some adventitious solvent.

A “pharmaceutical composition” refers to a composition which may comprise an ionizable lipid of the disclosure and a medium generally accepted in the art for the delivery of the biologically active compound to mammals, e.g., humans. Such a medium includes pharmaceutically acceptable carriers, diluents or excipients therefor.

“Effective amount” or “therapeutically effective amount” refers to that amount of an ionizable lipid of the disclosure which, when administered to a mammal, such as a human, is sufficient to effect treatment in the mammal, such as a human. The amount of a lipid nanoparticle of the disclosure which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, such as a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the disclosure, or their pharmaceutically acceptable salts may contain one or more stereocenters and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and(S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

In the following description, certain specific details are set forth to provide a thorough understanding of various embodiments of the disclosure. However, one of ordinary skill in the art will understand that the disclosure may be practiced without these details.

### Exemplary Lipid Compounds

In some embodiments, disclosed are ionizable lipids of Formula (LA-I):

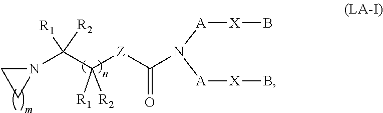

pharmaceutically acceptable salts, thereof, and stereoisomers of any of the foregoing, wherein
   each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ are taken together to form a cyclic ring;
   each of R₁₀ and R₁₁ is independently H, C₁-C₃ branched or unbranched alkyl, C₂-C₃ branched or unbranched alkenyl; or R₁₀ and R₁₁ are taken together to form a heterocyclic ring;
   m is 1, 2, 3, 4, 5, 6, 7 or 8;
   n is 0, 1, 2, 3 or 4;
   Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, SH;
   A is each independently C₁-C₁₆ branched or unbranched alkyl, optionally substituted with heteroatom or substituted with OH, SH, or halogen;
   B is each independently C₁-C₁₆ branched or unbranched alkyl, optionally substituted with heteroatom or substituted with OH, SH, or halogen;
   each X is independently X′,

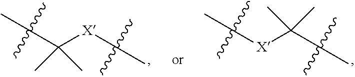

provided that at least one X in the formula is

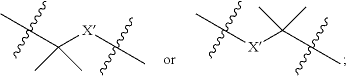

and
X′ is a biodegradable moiety.

In some embodiments, each X is

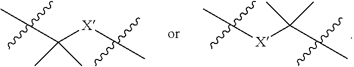

In some embodiments, X′ is —OCO—, —COO—, —NR⁷CO—, —CONR⁷—, —C(O—R₁₃)—O-(acetal), —COO(CH₂)ₛ—, —CONH(CH₂)ₛ—, —C(O—R₁₃)—O—(CH₂)ₛ—; wherein R⁷ is H or C₁-C₃ alkyl; and R₁₃ is C₃-C₁₀ alkyl.

In some embodiments, at least one X in the formula is

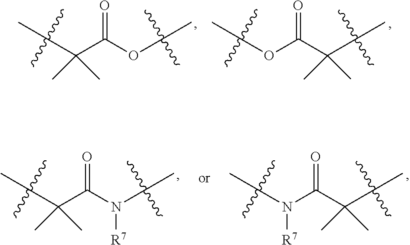

wherein R⁷ is H or methyl. In one embodiment, each X is

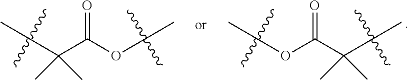

In one embodiment, each X is

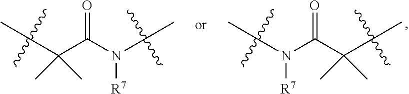

wherein R⁷ is H or methyl.

In some embodiments, the disclosure relates to ionizable lipids of Formula (LA-II):

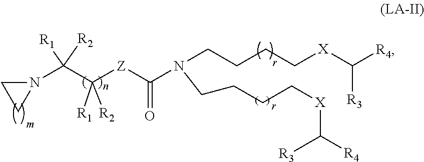

pharmaceutically acceptable salts thereof, and stereoisomers of any of the foregoing,
wherein
   R₁ is each independently H, C₁-C₃ alkyl, OH, halogen, SH, or NR₁₀R₁₁; R₁ and R₂ can be taken together to form a cyclic ring; R₁₀ and R₁₁ are each independently H, C₁-C₃ alkyl, and R₁₀ and R₁₁ can be taken together to form a heterocyclic ring;
   R₂ is each independently H, C₁-C₃ alkyl, OH, halogen, SH, or NR₁₀R₁₁; R₁ and R₂ can be taken together to form a cyclic ring; R₁₀ and R₁₁ are each independently H, C₁-C₃ alkyl, and R₁₀ and R₁₁ can be taken together to form a heterocyclic ring;
   m is 1, 2, 3, 4, 5, 6, 7 or 8;
   n is 0, 1, 2, 3 or 4;
   r is each independently 0, 1, 2, 3, 4, 5, 6, 7 or 8;
   R₃ is each independently H, or C₃-C₁₀ alkyl;
   R₄ is each independently H, or C₃-C₁₀ alkyl; provided that at least one of R₃ and R₄ is not H;
   Z is absent, O, S, or NR₁₂; wherein R₁₂ is C₁-C₇ alkyl;
   each X is independently X′,

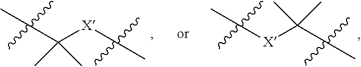

provided that at least one X in the formula is

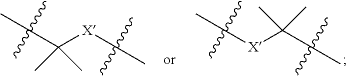

and
X′ is a biodegradable moiety.

In some embodiments, each X is

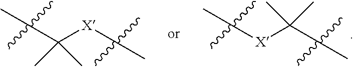

In some embodiments, X′ is —OCO—, —COO—, —NR⁷CO—, —CONR⁷—, —C(O—R₁₃)—O-(acetal), —COO(CH₂)ₛ—, —CONH(CH₂)ₛ—, —C(O—R₁₃)—O—(CH₂)ₛ—; wherein R⁷ is H or C₁-C₃ alkyl; and R₁₃ is C3-C10 alkyl.

In some embodiments, at least one X in the formula is

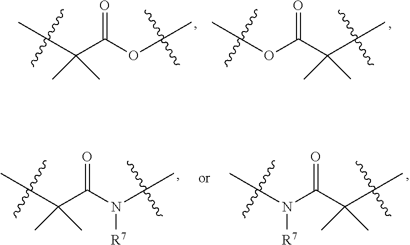

wherein R⁷ is H or methyl. In one embodiment, each X is

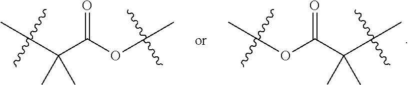

In one embodiment, each X is

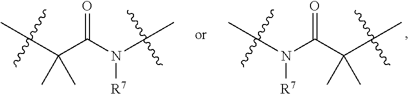

wherein R⁷ is H or methyl.

In some embodiments, the disclosure relates to ionizable lipids of Formula (LA-III):

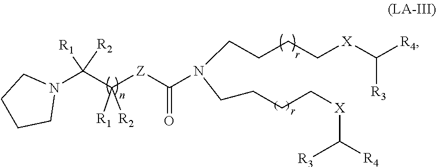

pharmaceutically acceptable salts, thereof, and stereoisomers of any of the foregoing,
wherein
   each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ are taken together to form a cyclic ring;
   each of R₁₀ and R₁₁ is independently H, C₁-C₃ branched or unbranched alkyl, C₂-C₃ branched or unbranched alkenyl; or R₁₀ and R₁₁ are taken together to form a heterocyclic ring;
   R₂ is each independently H, C₁-C₃ alkyl, OH, halogen, SH, or NR₁₀R₁₁; R₁ and R₂ can be taken together to form a cyclic ring; R₁₀ and R₁₁ are each independently H, C₁-C₃ alkyl, and R₁₀ and R₁₁ can be taken together to form a heterocyclic ring;
   n is 0, 1, 2, 3 or 4;
   r is each independently 0, 1, 2, 3, 4, 5, 6, 7 or 8;
   R₃ is each independently H, or C₃-C₁₀ alkyl;
   R₄ is each independently H, or C₃-C₁₀ alkyl; provided that at least one of R₃ and R₄ is not H;
   Z is absent, O, S, or NR₁₂; wherein R₁₂ is C₁-C₇ alkyl;
   each X is independently X′,

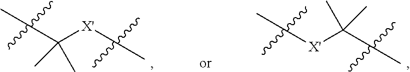

provided that at least one X in the formula is

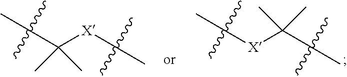

and
X′ is a biodegradable moiety.

In some embodiments, each X is

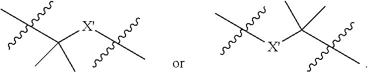

In some embodiments, X′ is —OCO—, —COO—, —NR⁷CO—, —CONR⁷—, —C(O—R₁₃)—O-(acetal), —COO(CH₂)ₛ—, —CONH(CH₂)ₛ—, —C(O—R₁₃)—O—(CH₂)ₛ—; wherein R⁷ is H or C₁-C₃ alkyl; and R₁₃ is C3-C10 alkyl.

In some embodiments, at least one X in the formula is

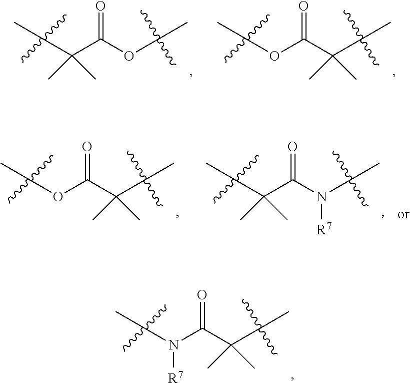

wherein R⁷ is H or methyl. In one embodiment, each X is

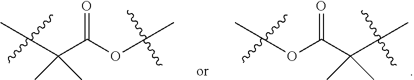

In one embodiment, each X is

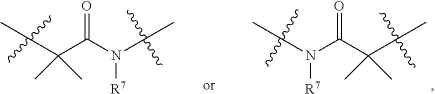

wherein R⁷ is H or methyl.

In some embodiments, the disclosure relates to ionizable lipids of Formula (LA-IV):

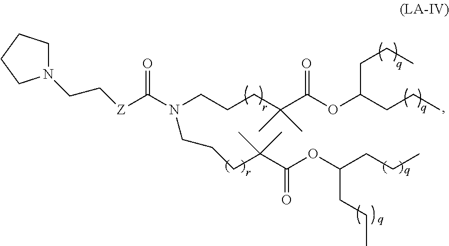

pharmaceutically acceptable salts, thereof, and stereoisomers of any of the foregoing,
wherein
r is each independently 0, 1, 2, 3, 4, 5, 6, 7 or 8;
q is each independently C₁-C₁₀ alkyl; and
Z is absent, O, S, or NR₁₂, wherein R₁₂ is C₁-C₇ alkyl.

In some embodiments, Z is absent.

In some embodiments, Z is S.

In some embodiments, Z is O.

In some embodiments, Z is NH.

In some embodiments, r is 3.

In some embodiments, r is 4.

In some embodiments, q is 3.

In some embodiments, q is 4.

In some embodiments, Z is absent, r is 4 and q is 4.

In some embodiments, the disclosure relates to ionizable lipids of Formula (LA-V):

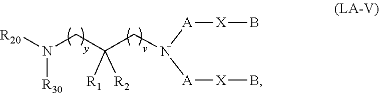

pharmaceutically acceptable salts thereof, and stereoisomers of any of the foregoing,
wherein
   R₁ is H, C₁-C₃ alkyl, OH, halogen, SH, or NR₁₀R₁₁;
   R₂ is OH, halogen, SH, or NR₁₀R₁₁; or R₁ and R₂ can be taken together to form a cyclic ring;
   R₁₀ and R₁₁ are each independently H or C₁-C₃ alkyl; or R₁₀ and R₁₁ can be taken together to form a heterocyclic ring;
   R₂₀ and R₃₀ are each independently H, C₁-C₅ branched or unbranched alkyl, C₂-C₅ branched or unbranched alkenyl; or R₂₀ and R₃₀ can be taken together to form a cyclic ring;
   each of v and y is independently 1, 2, 3, or 4;
   each of A and B is independently C₁-C₁₆ branched or unbranched alkyl, or C₂-C₁₆ branched or unbranched alkenyl; optionally interrupted with heteroatom or substituted with OH, SH, or halogen;
   each X is independently X′,

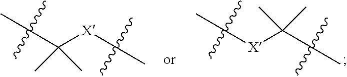

provided that at least one X in the formula is

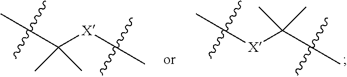

and
X′ is a biodegradable moiety.

In some embodiments, each X is

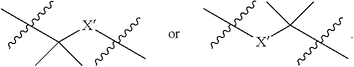

In some embodiments, X′ is —OCO—, —COO—, —NR⁷CO—, —CONR⁷—, —C(O—R₁₃)—O-(acetal), —COO(CH₂)ₛ—, —CONH(CH₂)ₛ—, —C(O—R₁₃)—O—(CH₂)ₛ—; wherein R⁷ is H or C₁-C₃ alkyl; and R₁₃ is C3-C10 alkyl.

In some embodiments, at least one X in the formula is

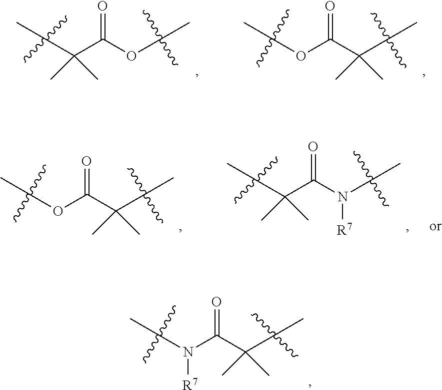

wherein R⁷ is H or methyl. In one embodiment, each X is

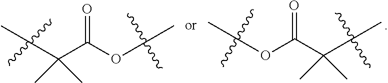

In one embodiment, each X is

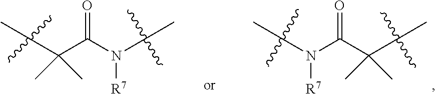

wherein R⁷ is H or methyl.

In some embodiments, the disclosure relates to ionizable lipids of Formula (LA-VI):

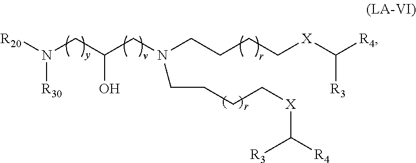

pharmaceutically acceptable salts thereof, and stereoisomers of any of the foregoing,
wherein
   R₂₀ and R₃₀ are each independently H, C₁-C₅alkyl; R₂₀ and R₃₀ can be taken together to form a cyclic ring;
   v is 1, 2, 3, or 4;
   y is 1, 2, 3, or 4;
   R₃ is each independently H, or C₃-C₁₀ alkyl;
   R₄ is each independently H, or C₃-C₁₀ alkyl; provided that at least one of R₃ and R₄ is not H;
   r is each independently 0, 1, 2, 3, 4, 5, 6, 7 or 8; and
   each X is independently X′,

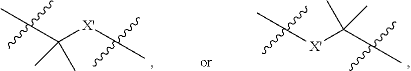

provided that at least one X in the formula is

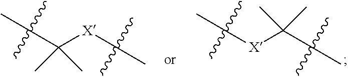

and
X′ is —OCO—, —COO—, —NR⁷CO—, —CONR⁷—, —C(O—R₁₃)—O-(acetal), —COO(CH₂)ₛ—, —CONH(CH₂)ₛ—, or —C(O—R₁₃)—O—(CH₂)ₛ—; wherein R⁷ is H or C₁-C₃ alkyl; and R₁₃ is C3-C10 alkyl.

In some embodiments, each X is

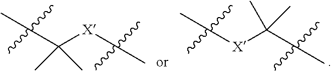

In some embodiments, at least one X in the formula is

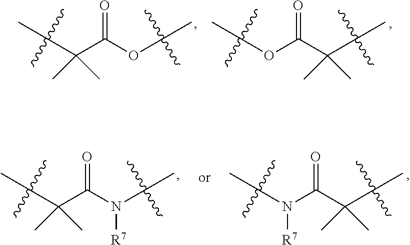

wherein R⁷ is H or methyl. In one embodiment, each X is

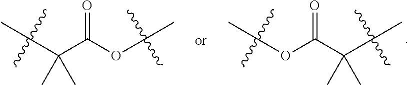

In one embodiment, each X is

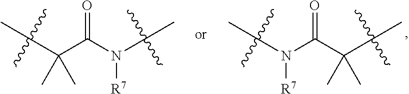

wherein R⁷ is H or methyl.

In some embodiments, the disclosure relates to ionizable lipids of Formula (LA-VII):

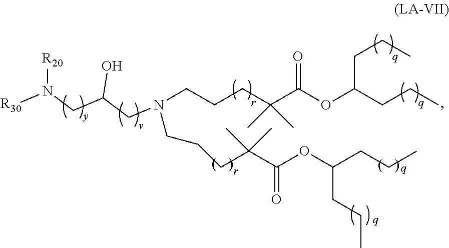

pharmaceutically acceptable salts thereof, and stereoisomers of any of the foregoing,
wherein
   R₂₀ and R₃₀ are each independently H, C₁-C₅alkyl; R₂₀ and R₃₀ can be taken together to form a cyclic ring;
   v is 1, 2, 3, or 4;
   y is 1, 2, 3, or 4;
   r is each independently 0, 1, 2, 3, 4, 5, 6, 7 or 8; and
   q is each independently C₁-C₁₀ alkyl.

In some embodiments, r is 3.

In some embodiments, r is 4.

In some embodiments, q is 3.

In some embodiments, q is 4.

In some embodiments, r is 4 and q is 4.

In some embodiments, B or

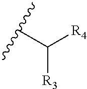

is selected from:

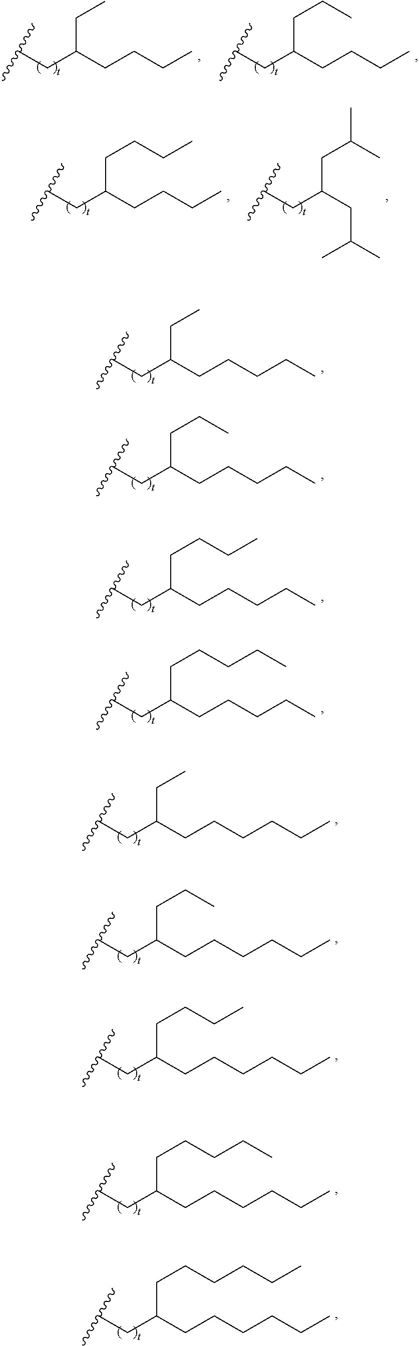

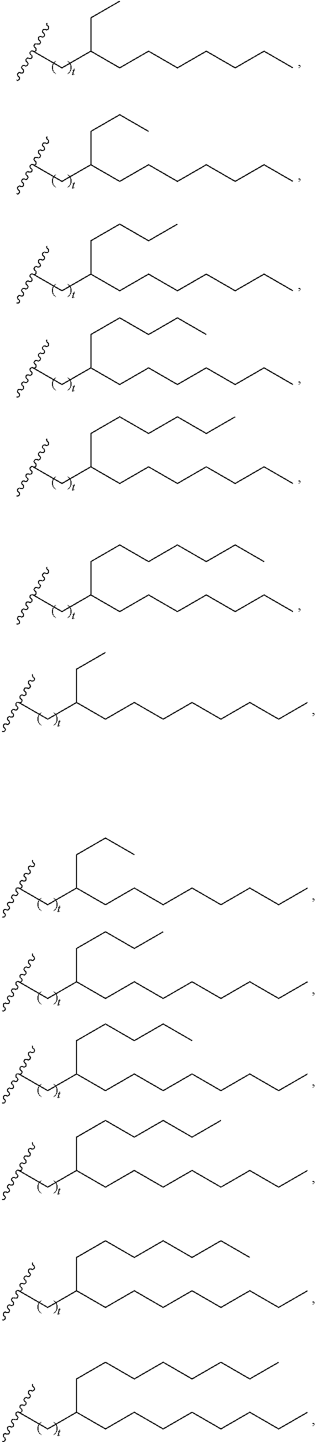

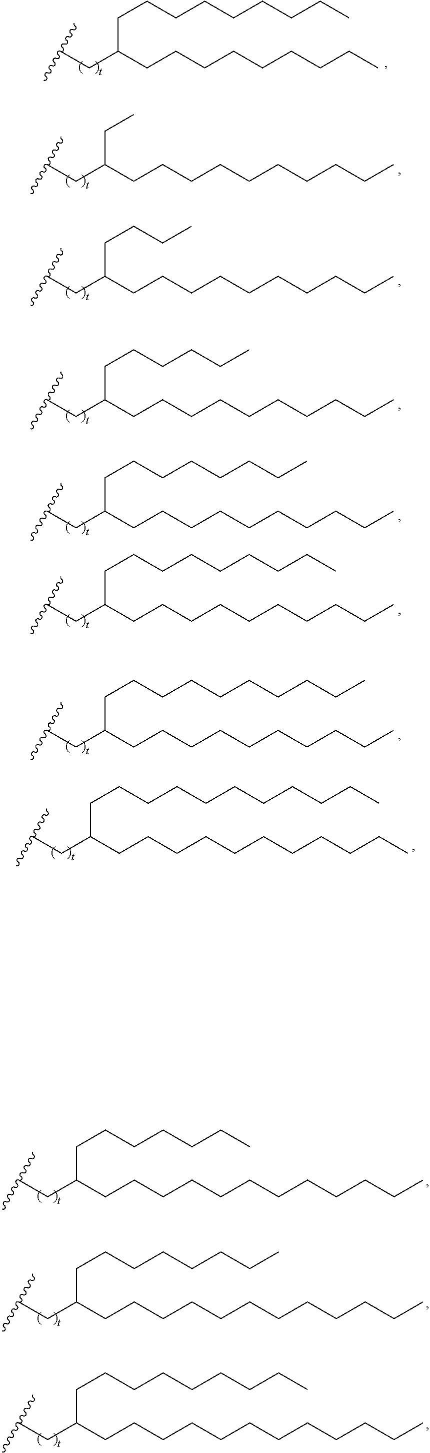

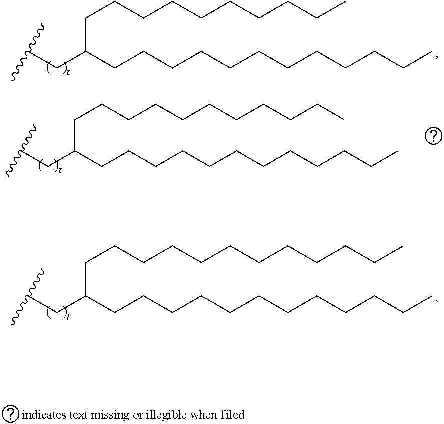

wherein t is 0, 1, 2, 3, 4, or 5.

In some embodiments, disclosed are ionizable lipids of Formula (LB-I):

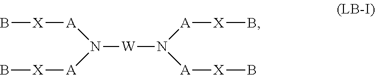

a pharmaceutically acceptable salt thereof, or a stereoisomer of any of the foregoing,
wherein
   each A is independently C₁-C₁₆ branched or unbranched alkylene or C₁-C₁₆ branched or unbranched alkenylene, optionally substituted with heteroatom or substituted with OH, SH, or halogen;
   each B is independently C₁-C₂₀ branched or unbranched alkyl or C₁-C₂₀ branched or unbranched alkenyl, optionally substituted with heteroatom or substituted with OH, SH, or halogen;
   each X is independently X′,

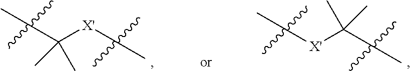

provided that at least one X in the formula is

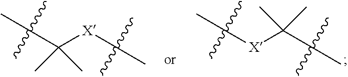

and
X′ is a biodegradable moiety; and
W is

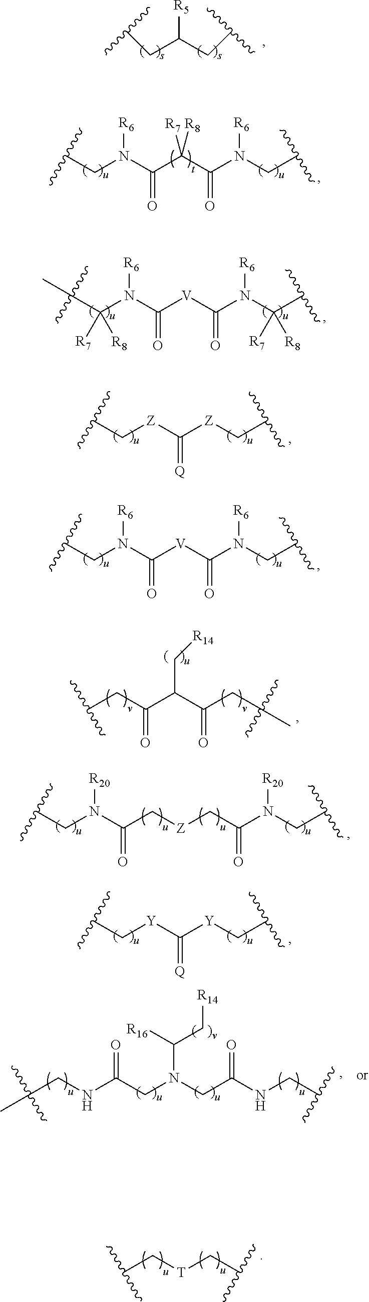

wherein
   R₅ is (CH₂)ₛOH, OH, SH, NR₁₀R₁₁;
   each R₆ is independently H, C₁-C₃ branched or unbranched alkyl, C₂-C₃ branched or unbranched alkenyl, or cycloalkyl;
   each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ are independently H or C₁-C₃ alkyl, or R₁₀ and R₁₁ are taken together to form a heterocyclic ring;
   each R₂₀ is independently H, or C₁-C₃ branched or unbranched alkyl;
   R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
   R₁₆ is H, ═O, ═S, or CN;
   each of s, u, and t is independently 1, 2, 3, 4, or 5;
   each v is independently 0, 1, 2, 3, 4, or 5;
   each Z is independently absent, O, S, or NR₁₂, wherein R₁₂ is H, C₁-C₇ branched or unbranched alkyl, or C₂-C₇ branched or unbranched alkenyl;
   each Y is a divalent heterocyclic;
   Q is O, S, CH₂, or NR₁₃, wherein each R₁₃ is H, C₁-C₅alkyl; and
   V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups.

In some embodiments, each X is

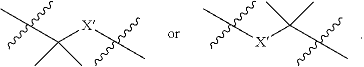

In some embodiments, X′ is —OCO—, —COO—, —NR⁷CO—, —CONR⁷—, —C(O—R₁₃)—O—, —COO(CH₂)ᵣ—, —CONH(CH₂)ᵣ—, or —C(O—R₁₃)—O—(CH₂)ᵣ, —O(CO)O—, wherein R⁷ is H or C₁-C₃ alkyl; and R₁₃ is branched or unbranched C₃-C₁₀ alkyl and r is 1, 2, 3, 4, or 5.

In some embodiments, at least one X in the formula is

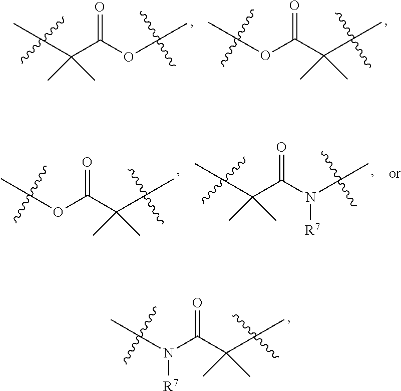

wherein R⁷ is H or methyl. In one embodiment, each X is

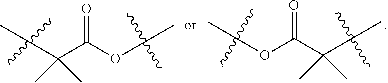

In one embodiment, each X is

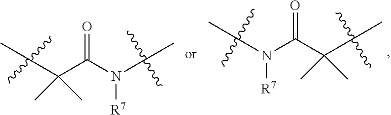

wherein R⁷ is H or methyl.

In some embodiments, the heterocyclic is a piperazine, piperazine dione, piperazine-2,5-dione, piperidine, pyrrolidine, piperidinol, dioxopiperazine, bis-piperazine, aromatic or heteroaromatic.

In some embodiments, the disclosure relates to ionizable lipids of Formula (LB-II):

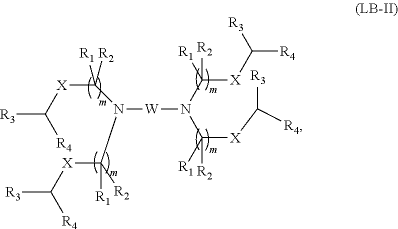

pharmaceutically acceptable salts thereof, and stereoisomers of any of the foregoing,
wherein
   each R₁ and each R₂ is independently H, C₁-C₃ branched or unbranched alkyl, OH, halogen, SH, or NR₁₀R₁₁, or
   each R₁ and each R₂ are independently taken together with the carbon atom(s) to which they are attached to form a cyclic ring;
   each R₁₀ and R₁₁ is independently H, C₁-C₃ branched or unbranched alkyl, or R₁₀ and R₁₁ are taken together to form a heterocyclic ring;
   m is 1, 2, 3, 4, 5, 6, 7, 8, 9 or 10;
   each X is independently X′,

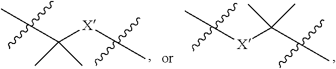

provided that at least one X in the formula is

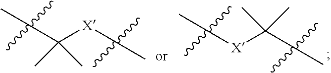

and
X′ is independently a biodegradable moiety;
each R³ and each R₄ is independently H, C₃-C₁₀ branched or unbranched alkyl, or C₃-C₁₀ branched or unbranched alkenyl; provided that at least one of R₃ and R₄ is not H;
W is

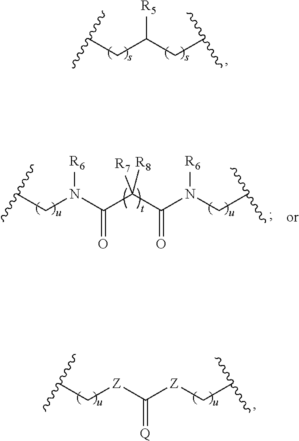

wherein
   R₅ is OH, SH, NR₁₀R₁₁;
   each R₆ is independently H, C₁-C₃ branched or unbranched alkyl, C₂-C₃ branched or unbranched alkenyl, or cycloalkyl;
   each R₇ and each R₈ is independently H, C₁-C₃ branched or unbranched alkyl, C₂-C₃ branched or unbranched alkenyl, halogen, OH, SH, NR₁₀R₁₁, wherein each R₁₀ and R₁₁ is independently H, C₁-C₃ alkyl, or each R₁₀ and each R₁₁ are taken together with the carbon atom(s) to which they are attached to form a heterocyclic ring;
   each s is independently 1, 2, 3, 4, or 5;
   each u is independently 1, 2, 3, 4, or 5;
   t is 1, 2, 3, 4 or 5;
   each Z is independently absent, O, S, or NR₁₂, wherein R₁₂ is H, C₁-C₇ branched or unbranched alkyl, or C₂-C₇ branched or unbranched alkenyl, provided that when Z is not absent, the adjacent R₁ and R₂ cannot be OH, NR₁₀R₁₁, or SH; and
   Q is O, S, CH₂, or NR₁₃, wherein each R₁₃ is H, C₁-C₅alkyl.

In some embodiments, each X is

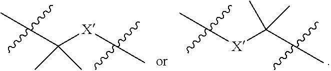

In some embodiments, X′ is —OC(O)—, —C(O)O—, —NR⁷C(O)—, —C(O)NR⁷—, —C(O—R₁₃)—O—, —C(O)O(CH₂)ᵣ—, —C(O)NH(CH₂)ᵣ—, —CON(R₁₃)—, or —C(O—R₁₃)—O—(CH₂)ᵣ, —OC(O)O—, wherein R⁷ is H or C₁-C₃ alkyl; and R₁₃ is branched or unbranched C₁-C₁₀ alkyl and r is 1, 2, 3, 4, or 5.

In some embodiments, at least one X in the formula is

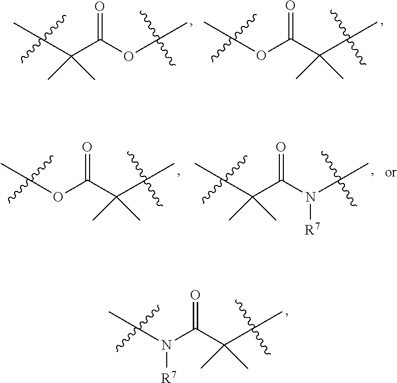

wherein R⁷ is H or methyl. In one embodiment, each X is

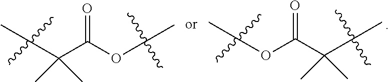

In one embodiment, each X is

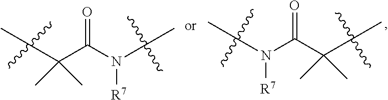

wherein R⁷ is H or methyl.

In some embodiments, W is

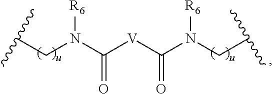

wherein
   V is C₂-C₆ alkylene, C₂-C₁₀ alkenylene, C₂-C₁₀ alkynylene, or C₂-C₁₀ heteroalkylene;
   each R₆ is independently H, C₁-C₃ branched or unbranched alkyl, C₂-C₃ branched or unbranched alkenyl, or cycloalkyl; and
   each u is independently 2, 3, 4, or 5.

In some embodiments, in formula (HB-I), W is

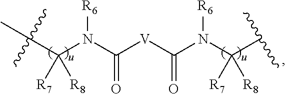

wherein:
each R₆ is independently H or methyl;
each R₇ is independently H;
each R₈ is methyl;
each u is independently 1, 2, or 3; and
V is C₂-C₆ alkenylene.

In some embodiments, the disclosure relates to ionizable lipids of Formula (LB-III):

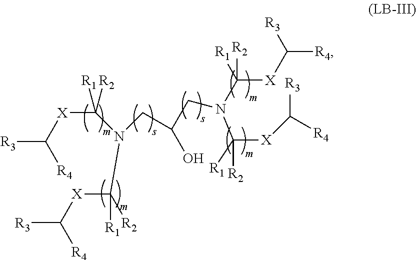

pharmaceutically acceptable salts thereof, and stereoisomers of any of the foregoing,
wherein
   each R₁ and each R₂ is independently H, C₁-C₃ branched or unbranched alkyl, OH, halogen, SH, or NR₁₀R₁₁, or
   each R₁ and each R₂ are independently taken together with the carbon atom(s) to which they are attached to form a cyclic ring;
   each R₁₀ and R₁₁ is independently H, C₁-C₃ branched or unbranched alkyl, or R₁₀ and R₁₁ are taken together to form a heterocyclic ring;
   each R₃ and each R₄ is independently H, C₃-C₁₀ branched or unbranched alkyl, or C₃-C₁₀ branched or unbranched alkenyl, provided that at least one of R₃ and R₄ is not H;
   each X is independently X′,

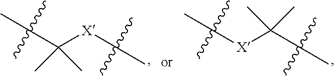

provided that at least one X in the formula is

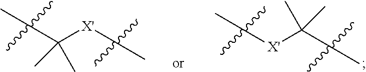

and
X′ is independently a biodegradable moiety;
each m is independently 1, 2, 3, 4, 5, 6, 7, 8, 9 or 10; and
each s is independently 1, 2, 3, 4, or 5.

In some embodiments, each X is

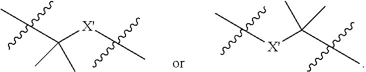

In some embodiments, X′ is —OC(O)—, —C(O)O—, —NR⁷C(O)—, —C(O)NR⁷—, —C(O—R₁₃)—O—, —C(O)O(CH₂)ᵣ, —C(O)NH(CH₂)ᵣ, —CON(R₁₃)—, or —C(O—R₁₃)—O—(CH₂)ᵣ, —OC(O)O—, wherein R⁷ is H or C₁-C₃ alkyl; and R₁₃ is branched or unbranched C₁-C₁₀ alkyl and r is 1, 2, 3, 4, or 5.

In some embodiments, at least one X in the formula is

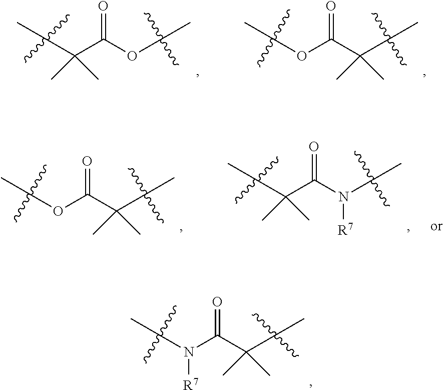

wherein R⁷ is H or methyl. In one embodiment, each X is

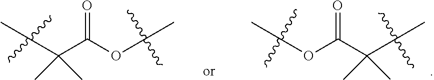

In one embodiment, each X is

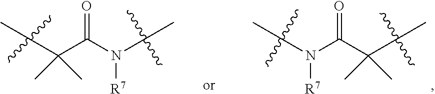

wherein R⁷ is H or methyl.

In some embodiments, the disclosure relates to ionizable lipids of Formula (LB-IV):

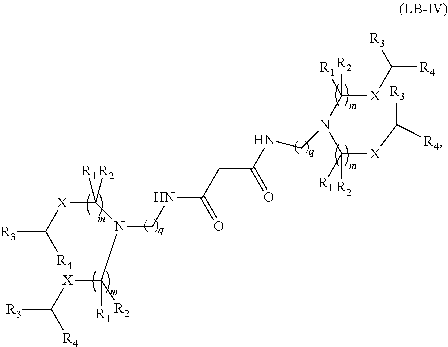

pharmaceutically acceptable salts, thereof, and stereoisomers of any of the foregoing,
wherein
   each R₁ and each R₂ is independently H, C₁-C₃ branched or unbranched alkyl, OH, halogen, SH, or NR₁₀R₁₁, or
   each R₁ and each R₂ are independently taken together with the carbon atom(s) to which they are attached to form a cyclic ring;
   each R₁₀ and R₁₁ is independently H, C₁-C₃ branched or unbranched alkyl, or R₁₀ and R₁₁ are taken together to form a heterocyclic ring;
   each R₃ and each R₄ is independently H, C₃-C₁₀ branched or unbranched alkyl, or C₃-C₁₀ branched or unbranched alkenyl, provided that at least one of R₃ and R₄ is not H;
   each X is independently X′,

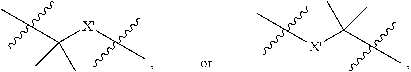

provided that at least one X in the formula is

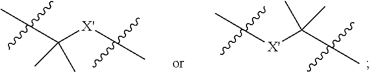

and
X′ is independently a biodegradable moiety;
each q is independently 2, 3, 4, or 5; and
each m is independently 1, 2, 3, 4, 5, 6, 7, 8, 9 or 10.

In some embodiments, each X is

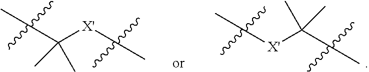

In some embodiments, X′ is —OC(O)—, —C(O)O—, —NR⁷C(O)—, —C(O)R⁷H—, —C(O—R₁₃)—O—, —C(O)O(CH₂)ᵣ, —C(O)NH(CH₂)ᵣ, —CON(R₁₃)—, or —C(O—R₁₃)—O—(CH₂)ᵣ, —OC(O)O—, wherein R⁷ is H or C₁-C₃ alkyl; and R₁₃ is branched or unbranched C₁-C₁₀ alkyl and r is 1, 2, 3, 4, or 5.

In some embodiments, at least one X in the formula is

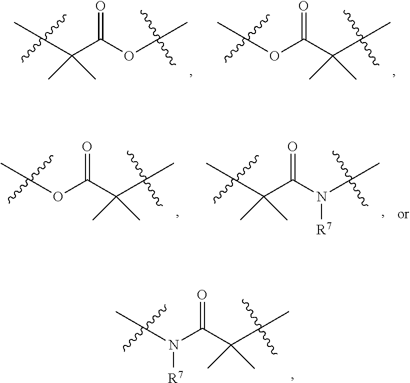

wherein R⁷ is H or methyl. In one embodiment, each X is

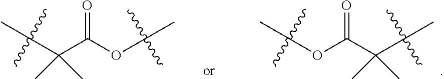

In one embodiment, each X is

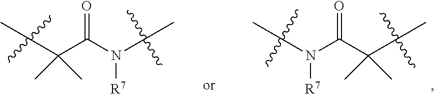

wherein R⁷ is H or methyl.

In some embodiments, the disclosure relates to ionizable lipids of Formula (LB-V):

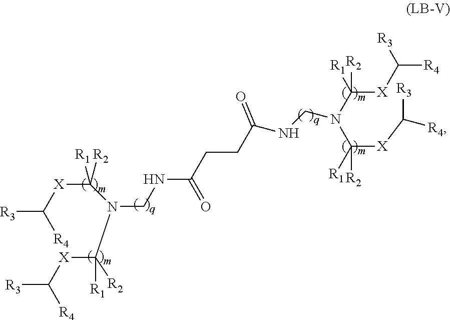

pharmaceutically acceptable salts, thereof, and stereoisomers of any of the foregoing,
wherein
   each R₁ and each R₂ is independently H, C₁-C₃ branched or unbranched alkyl, OH, halogen, SH, or NR₁₀R₁₁, or
   each R₁ and each R₂ are independently taken together with the carbon atom(s) to which they are attached to form a cyclic ring;
   each R₁₀ and R₁₁ is independently H, C₁-C₃ branched or unbranched alkyl, or R₁₀ and R₁₁ are taken together to form a heterocyclic ring;
   each R³ and each R₄ is independently H, C₃-C₁₀ branched or unbranched alkyl, or C₃-C₁₀ branched or unbranched alkenyl, provided that at least one of R₃ and R₄ is not H;
   each X is independently X′,

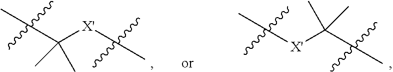

provided that at least one X in the formula is

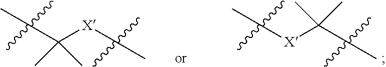

and
X′ is independently a biodegradable moiety;
each q is independently 2, 3, 4, or 5; and
each m is independently 1, 2, 3, 4, 5, 6, 7, 8, 9 or 10.

In some embodiments, each X is

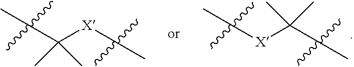

In some embodiments, X′ is —OC(O)—, —C(O)O—, —NR⁷C(O)—, —C(O)NR⁷—, —C(O—R₁₃)—O—, —C(O)O(CH₂)ᵣ, —C(O)NH(CH₂)ᵣ, —CON(R₁₃)—, or —C(O—R₁₃)—O—(CH₂)ᵣ, —OC(O)O—, wherein R⁷ is H or C₁-C₃ alkyl; and R₁₃ is branched or unbranched C₁-C₁₀ alkyl and r is 1, 2, 3, 4, or 5.

In some embodiments, at least one X in the formula is

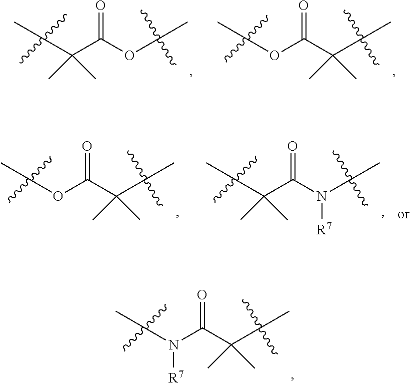

wherein R⁷ is H or methyl. In one embodiment, each X is

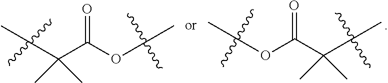

In one embodiment, each X is

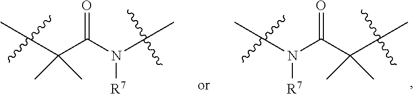

wherein R⁷ is H or methyl.

In some embodiments, the disclosure relates to ionizable lipids of Formula (LB-VI):

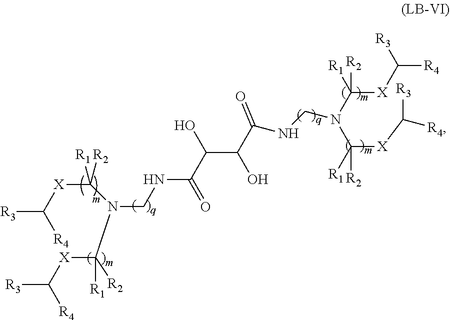

pharmaceutically acceptable salts thereof, and stereoisomers of any of the foregoing,
wherein
   each R₁ and each R₂ is independently H, C₁-C₃ branched or unbranched alkyl, OH, halogen, SH, or NR₁₀R₁₁, or
   each R₁ and each R₂ are independently taken together with the carbon atom(s) to which they are attached to form a cyclic ring;
   each R₁₀ and R₁₁ is independently H, C₁-C₃ branched or unbranched alkyl, or R₁₀ and R₁₁ are taken together to form a heterocyclic ring;
   each R³ and each R₄ is independently H, C₃-C₁₀ branched or unbranched alkyl, or C₃-C₁₀ branched or unbranched alkenyl, provided that at least one of R₃ and R₄ is not H;
   each X is independently X′,

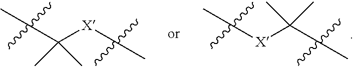

provided that at least one X in the formula is

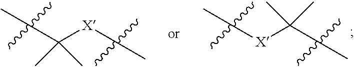

and
X′ is independently a biodegradable moiety;
each q is independently 2, 3, 4, or 5; and
each m is independently 1, 2, 3, 4, 5, 6, 7, 8, 9 or 10.

In some embodiments, each X is

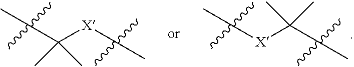

In some embodiments, X′ is —OC(O)—, —C(O)O—, —NR⁷C(O)—, —C(O)NR⁷—, —C(O—R₁₃)—O—, —C(O)O(CH₂)ᵣ, —C(O)NH(CH₂)ᵣ, —CON(R₁₃)—, or —C(O—R₁₃)—O—(CH₂)ᵣ, —OC(O)O—, wherein R⁷ is H or C₁-C₃ alkyl; and R₁₃ is branched or unbranched C₁-C₁₀ alkyl and r is 1, 2, 3, 4, or 5.

In some embodiments, at least one X in the formula is

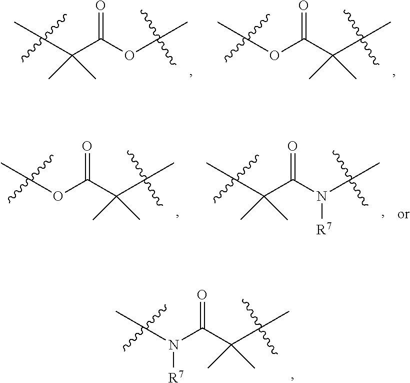

wherein R⁷ is H or methyl. In one embodiment, each X is

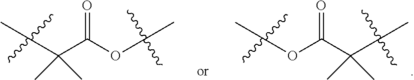

In one embodiment, each X is

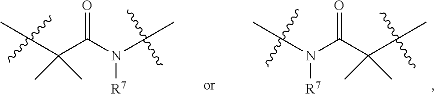

wherein R⁷ is H or methyl.

In some embodiments, the disclosure relates to ionizable lipids of Formula (LB-VII):

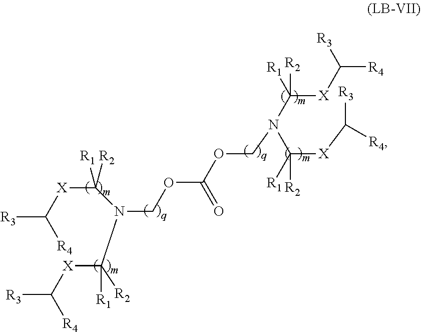

pharmaceutically acceptable salts thereof, and stereoisomers of any of the foregoing,
wherein
   each R₁ and each R₂ is independently H, C₁-C₃ branched or unbranched alkyl, OH, halogen, SH, or NR₁₀R₁₁, or
   each R₁ and each R₂ are independently taken together with the carbon atom(s) to which they are attached to form a cyclic ring;
   each R₁₀ and R₁₁ is independently H, C₁-C₃ branched or unbranched alkyl, or R₁₀ and R₁₁ are taken together to form a heterocyclic ring;
   each R³ and each R₄ is independently H, C₃-C₁₀ branched or unbranched alkyl, or C₃-C₁₀ branched or unbranched alkenyl, provided that at least one of R₃ and R₄ is not H;
   each X is independently X′,

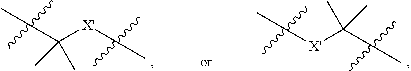

provided that at least one X in the formula is

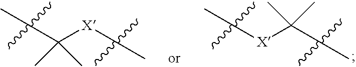

and
X′ is independently a biodegradable moiety;
each q is independently 2, 3, 4, or 5; and
each m is independently 1, 2, 3, 4, 5, 6, 7, 8, 9 or 10.

In some embodiments, each X is

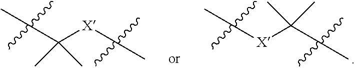

In some embodiments, X′ is —OC(O)—, —C(O)O—, —NR₁C(O)—, —C(O)NR⁷—, —C(O—R₁₃)—O—, —C(O)O(CH₂)ᵣ, —C(O)NH(CH₂)ᵣ, —CON(R₁₃)—, or —C(O—R₁₃)—O—(CH₂)ᵣ, —OC(O)O—, wherein R⁷ is H or C₁-C₃ alkyl; and R₁₃ is branched or unbranched C₁-C₁₀ alkyl and r is 1, 2, 3, 4, or 5.

In some embodiments, at least one X in the formula is

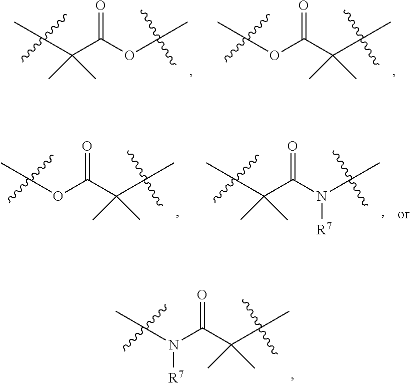

wherein R⁷ is H or methyl. In one embodiment, each X is

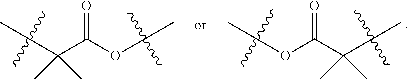

In one embodiment, each X is

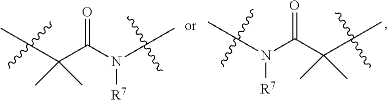

wherein R⁷ is H or methyl.

In some embodiments, B or

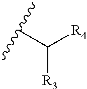

is selected from:

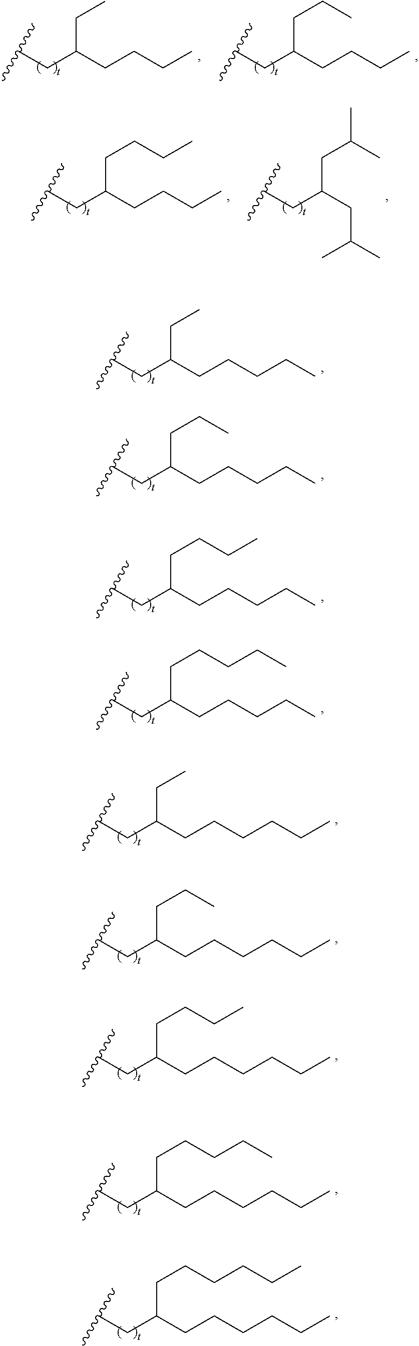

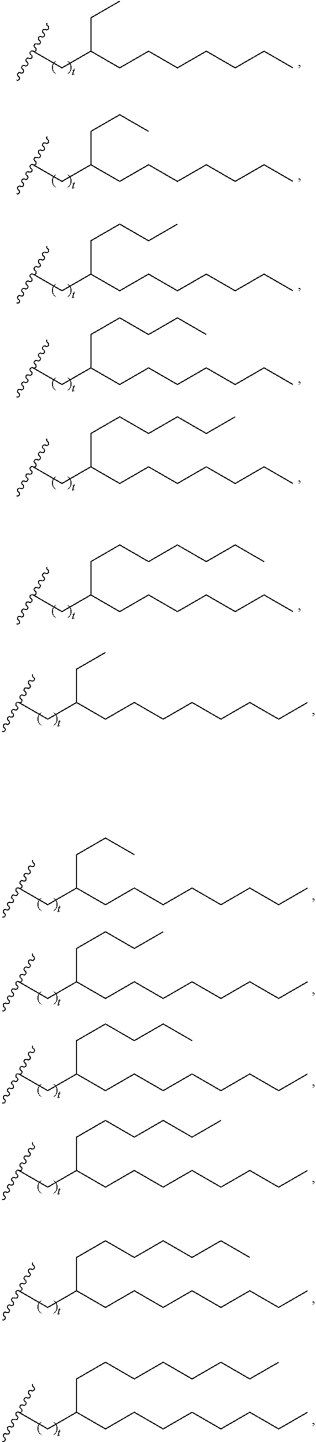

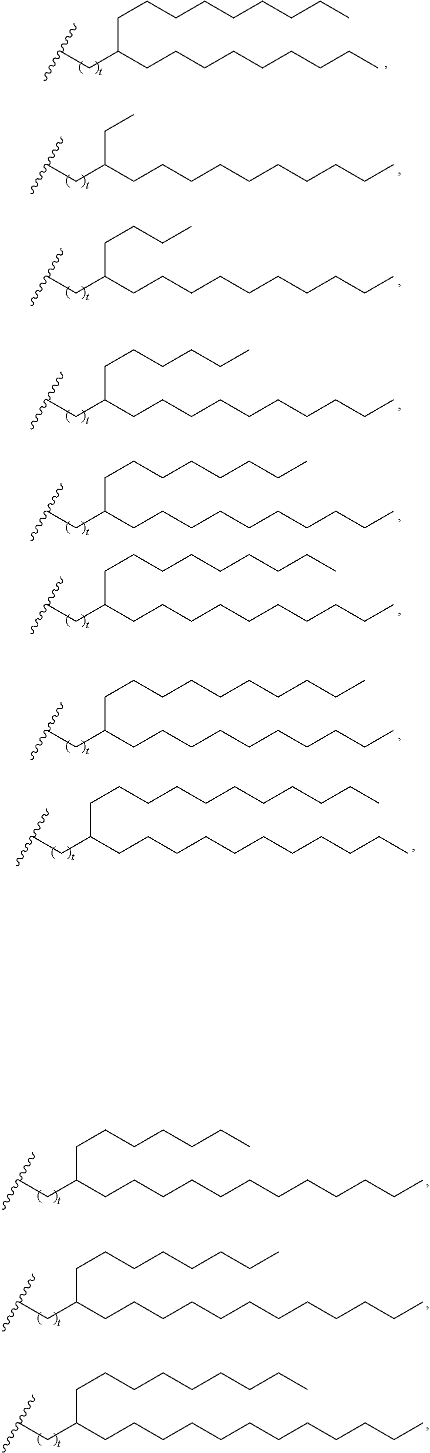

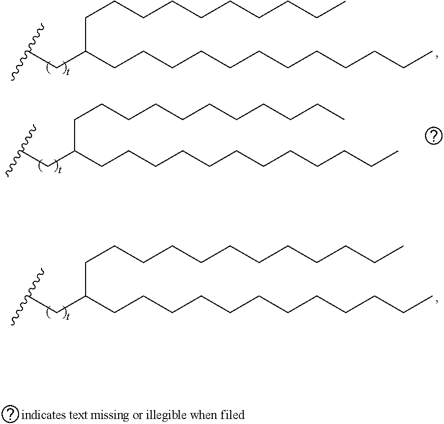

where t is 0, 1, 2, 3, 4, or 5.

In some embodiments, disclosed are ionizable lipids of Formula (LC-I):

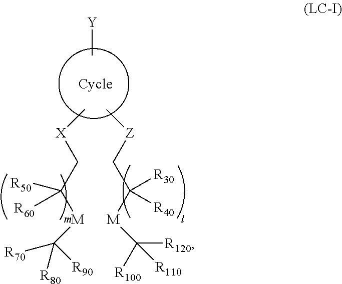

a pharmaceutically acceptable salt thereof, or a stereoisomer of any of the foregoing,
wherein:
   [symbol] is cyclic or heterocyclic moiety;
   Y is alkyl, hydroxy, hydroxyalkyl,

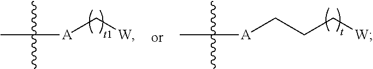

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S— or a bivalent heterocycle;
each of X and Z is independently absent, —O—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
each M is independently M′,

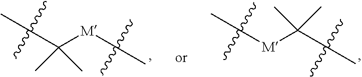

provided that at least one M in the formula is

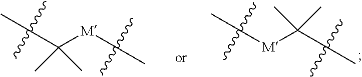

each M′ is independently a biodegradable moiety;
each of R₃₀, R₄₀, R₅₀, R₆₀, R₇₀, R₈₀, R₉₀, R₁₀₀, R₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t is 0, 1, 2, or 3;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl, or one of the following moieties:

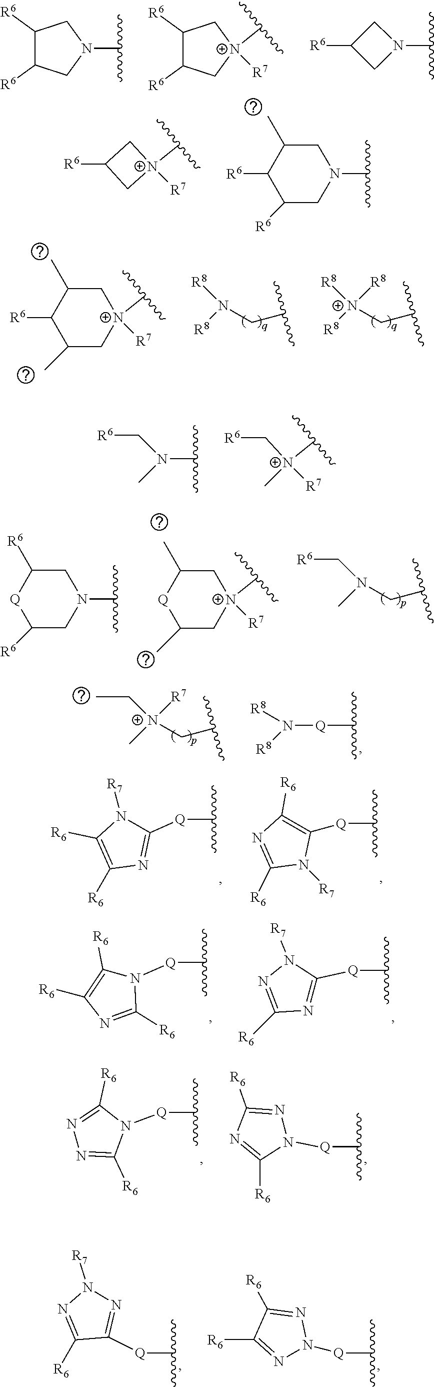

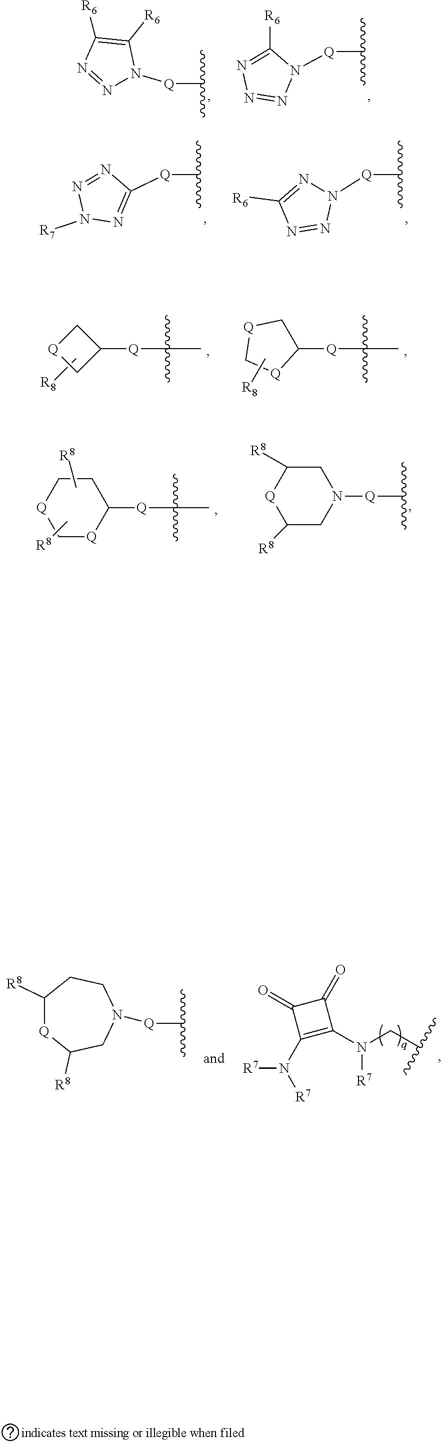

wherein
   each Q is independently absent, —O—, —C(O)—, —C(S)—, —C(O)O—, —(CH₂)_{q}—C(R⁷)₂, —C(O)N(R⁷)—, —C(S)N(R⁷)—, or —N(R⁷);
   R⁶ is independently H, alkyl, hydroxyl, hydroxyalkyl, alkoxy, —O-alkylene-O-alkyl, —O-alkylene-N(R⁷)₂, amino, alkylamino, aminoalkyl, N⁺(R⁷)₃-alkylene-Q-, thiol, or thiolalkyl;
   each R⁸ is independently H, alkyl, hydroxyalkyl, amino, aminoalkyl, alkylamino, heterocyclyl, heteroaryl, thiol, or thiolalkyl, or two R⁸ together with the nitrogen atom may form a ring, optionally substituted with one or more alkyl, hydroxy, hydroxyalkyl, alkoxy, alkylaminoalkyl, alkylamino, or aminoalkyl;
   q is 0, 1, 2, 3, 4, or 5; and
   p is 0, 1, 2, 3, 4, or 5.

In some embodiments, Y is hydroxy,

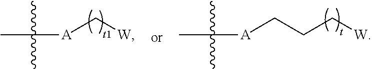

In some embodiments, each of R₇₀ and R₈₀ is H; and R₉₀ is C₁-C₁₅ branched or unbranched alkyl, C₁-C₁₅ branched or unbranched alkenyl, cycloalkyl or substituted cycloalkyl. In some embodiments, R₉₀ is C₁-C₁₅ branched or unbranched alkyl, C₁-C₁₅ branched or unbranched alkenyl. In some embodiments, R₉₀ is C₁-C₁₅ branched or unbranched alkyl. In some embodiments R₉₀ is C₁-C₁₂ branched or unbranched alkyl.

In some embodiments, R₇₀ is H; and each of R₈₀ and R₉₀ is independently C₁-C₁₅ branched or unbranched alkyl, C₁-C₁₅ branched or unbranched alkenyl, or cycloalkyl or substituted cycloalkyl. In some embodiments, each of R₈₀ and R₉₀ is independently C₁-C₁₅ branched or unbranched alkyl, C₁-C₁₅ branched or unbranched alkenyl. In some embodiments, each of R₈₀ and R₉₀ is independently C₁-C₁₅ branched or unbranched alkyl. In some embodiments, each of R₈₀ and R₉₀ is independently C₁-C₁₂ branched or unbranched alkyl. In some embodiments, each of R₅₀ and R₉₀ is independently C₁-C₈ branched or unbranched alkyl.

In some embodiments, R₁₀₀ is H; and each of R₁₀ and R₁₂₀ is independently C₁-C₁₅ branched or unbranched alkyl, C₁-C₁₅ branched or unbranched alkenyl, or cycloalkyl or substituted cycloalkyl. In some embodiments, each of R₁₀ and R₁₂₀ is independently C₁-C₁₅ branched or unbranched alkyl, C₁-C₁₅ branched or unbranched alkenyl. In some embodiments, each of R₁₁₀ and R₁₂₀ is independently C₁-C₁₅ branched or unbranched alkyl. In some embodiments, each of R₁₁₀ and R₁₂₀ is independently C₁-C₁₂ branched or unbranched alkyl. In some embodiments, each of R₁₁₀ and R₁₂₀ is independently C₁-C₈ branched or unbranched alkyl.

In some embodiments, disclosed are ionizable lipids of Formula (LC-IA) or (LC-IA-2):

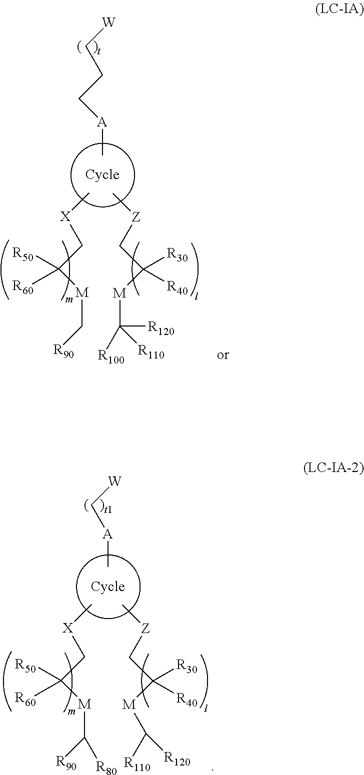

a pharmaceutically acceptable salt thereof, or a stereoisomer of any of the foregoing,
wherein:
   [symbol] is cyclic or heterocyclic moiety;
   A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
   X is absent, —O—, —CO—, —N(R⁷)—, —O-alkylene-, -alkylene-O—, —OC(O)—, —C(O)O—, —N(R⁷)C(O)—, —C(O)N(R⁷)—, or —S—;
   Z is absent, —O—, —N(R⁷)—, —O-alkylene-, -alkylene-O—, —OC(O)—, —C(O)O—, —N(R⁷)C(O)—, —C(O)N(R⁷)—, or —S—;
   each R⁷ is independently H, alkyl, alkenyl, cycloalkyl, hydroxy, alkoxy, hydroxyalkyl, alkylamino, alkylaminoalkyl, or aminoalkyl;
   each M is independently M′,

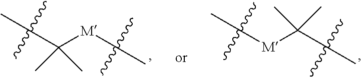

provided that at least one M in the formula is

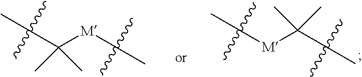

each M′ is independently a biodegradable moiety;
each of R₃₀, R₄₀, R₅₀, R₆₀, R₁₀₀, R₁₁₀, and R₁₂₀ is independently H, C₁-C₁₆ branched or unbranched alkyl, or C₁-C₁₆ branched or unbranched alkenyl, optionally interrupted with heteroatom or substituted with OH, SH, or halogen;
R₉₀ is C₁-C₁₅ branched or unbranched alkyl, C₁-C₁₅ branched or unbranched alkenyl, or cycloalkyl or substituted cycloalkyl;
t is 0, 1, 2, or 3;
t1 is an integer from 0 to 10;
l is an integer from 1 to 10;
m is an integer from 1 to 10; and
W is hydroxyl, or a bivalent heterocycle hydroxyalkyl, substituted or unsubstituted amino, substituted or unsubstituted aminocarbonyl, or substituted or unsubstituted heterocyclyl or heteroaryl, or one of the following moieties:

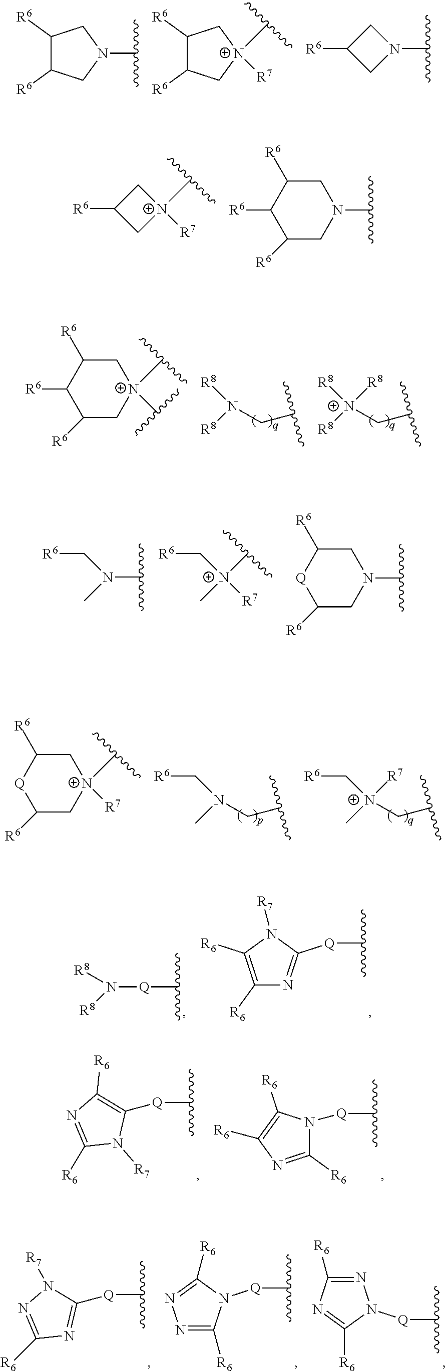

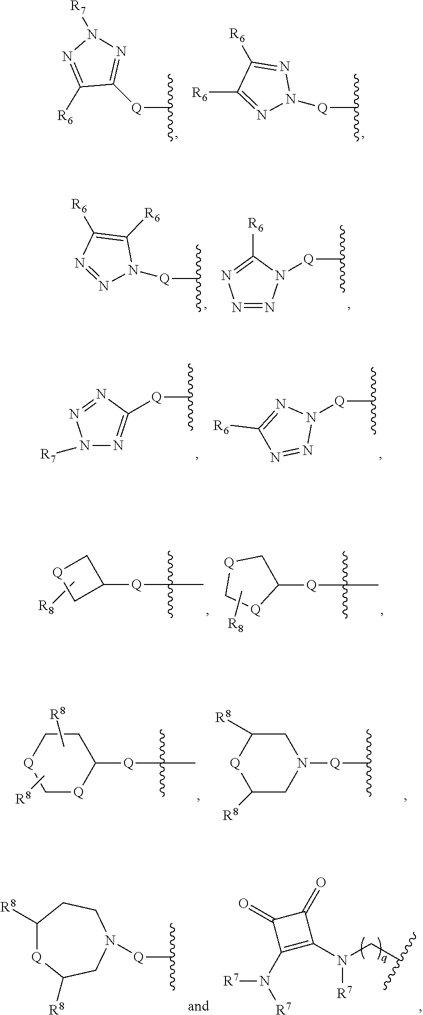

wherein
   each Q is independently absent, —O—, —C(O)—, —C(S)—, —C(O)O—, —(CH₂)_{q}—C(R⁷)₂, —C(O)N(R⁷)—, —C(S)N(R⁷)—, or —N(R⁷);
   R⁶ is independently H, alkyl, hydroxyl, hydroxyalkyl, alkoxy, —O-alkylene-O-alkyl, —O-alkylene-N(R⁷)₂, amino, alkylamino, aminoalkyl, N⁺(R⁷)₃-alkylene-Q-, thiol, or thiolalkyl;
   each R⁸ is independently H, alkyl, hydroxyalkyl, amino, aminoalkyl, alkylamino, heterocyclyl, heteroaryl, thiol, or thiolalkyl, or two R⁸ together with the nitrogen atom may form a ring, optionally substituted with one or more alkyl, hydroxy, hydroxyalkyl, alkoxy, alkylaminoalkyl, alkylamino, or aminoalkyl;
   q is 0, 1, 2, 3, 4, or 5; and
   p is 0, 1, 2, 3, 4, or 5.

In some embodiments, A is absent, —O—, —N(R⁷)—, —C(O)N(R⁷)—, —N(R⁷)C(O)—, —OC(O)—, or —C(O)O—. In one embodiment, A is absent. In one embodiment, A is —O—. In one embodiment, A is —N(R⁷)—, wherein R⁷ is H or C₁-C₃ alkyl. In one embodiment, A is —OC(O)— or —C(O)O—. In one embodiment, A is —NHC(O)— or —C(O)NH—.

Embodiments regarding various variables in the formulas (LC-I) and (LC-IA) are further discussed below.

In some embodiments, X is absent, —O—, or —C(O)—.

In some embodiments, Z is —O—, —C(O)O—, or —OC(O)—.

In some embodiments, each of R₃₀, R₄₀, R₅₀, and R₆₀ is H or C₁-C₄ branched or unbranched alkyl.

In some embodiments, each of R₃₀, R₄₀, R₅₀, and R₆₀ is H.

In some embodiments, R₉₀ is C₁-C₁₅ branched or unbranched alkyl, C₁-C₁₅ branched or unbranched alkenyl. In some embodiments, R₉₀ is C₁-C₁₅ branched or unbranched alkyl. In some embodiments, R₉₀ is C₁-C₁₂ branched or unbranched alkyl. In some embodiments, R₉₀ is C₁-C₈ branched or unbranched alkyl.

In some embodiments, R₁₀₀ is H; and each of R₁₀ and R₁₂₀ is independently C₁-C₁₅ branched or unbranched alkyl, C₁-C₁₅ branched or unbranched alkenyl, or cycloalkyl or substituted cycloalkyl. In some embodiments, each of R₁₁₀ and R₁₂₀ is independently C₁-C₁₅ branched or unbranched alkyl, C₁-C₁₅ branched or unbranched alkenyl. In some embodiments, each of R₁₁₀ and R₁₂₀ is independently C₁-C₁₅ branched or unbranched alkyl. In some embodiments, each of R₁₁₀ and R₁₂₀ is independently C₁-C₁₂ branched or unbranched alkyl. In some embodiments, each of R₁₁₀ and R₁₂₀ is independently C₁-C₈ branched or unbranched alkyl.

In some embodiments, 1 is from 3 to 10, from 3 to 7, or from 4 to 7.

In some embodiments, m is from 4 to 10, from 5 to 8, from 1 to 7, from 3 to 7, or from 1 to 5.

In some embodiments, 1 is 4, 5, 6, 7, 8, 9 or 10. In some embodiments, m is 4, 5, 6, 7, 8, 9 or 10.

In some embodiments, 1 is 4, 5, 6, or 7. In some embodiments, m is 3, 4, or 5. In some embodiments, m is 5, 6, 7, or 8.

In some embodiments, each M is

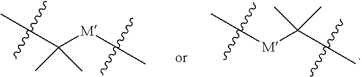

In some embodiments, M′ is —OC(O)—, —C(O)O—, —N(R⁷)C(O)—, —C(O)N(R⁷)—, —C(O—R₁₃)—O—, —C(O)O(CH₂)ᵣ, —C(O)N(R⁷)(CH₂)ᵣ, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl, and r is 1, 2, 3, 4, or 5.

In some embodiments, at least one M in the formula is

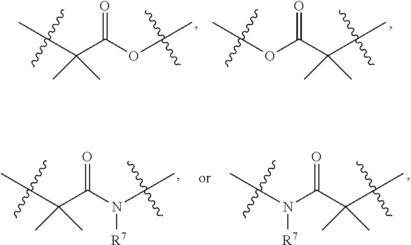

wherein R⁷ is H or methyl. In one embodiment, each M is

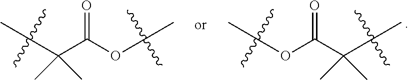

In one embodiment, each M is

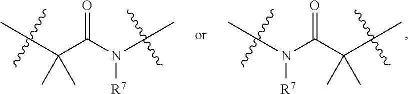

wherein R⁷ is H or methyl.

In some embodiments, [symbol] is a 5- to 7-membered, monocyclic ring. In some embodiments, [symbol] is a 5- to 7-membered, monocyclic, cycloalkane ring. In some embodiments, [symbol] is a 5- to 7-membered, monocyclic, heterocycle ring.

In some embodiments, [symbol] is a bicyclic or tricyclic ring, i.e., containing two or more rings, such as fused rings.

In some embodiments, [symbol] has a structure of formula

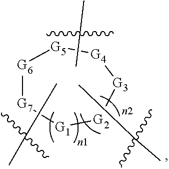

wherein:
each of G₁, G₂, G₃, G₄, G₅, and G₆, is independently C(R′)(R″), O, or N, provided that no more than two of G₁-G₆ are O or N;
R′ and R″ are each independently absent, H, alkyl, or two R′ from the two neighboring G together form a second 5- to 7-membered cyclic or heterocylic ring; and
n₁ and n₂ are each independently 0 or 1.

In some embodiments, [symbol] is selected from pyrrolidine, piperidine, piperazine, cyclohexane, cyclopentane, tetrahydrofuran, tetrahydropyran, morpholine, and dioxane.

In some embodiments, [symbol] has a structure of

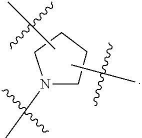

In one embodiment, [symbol] has a structure of.

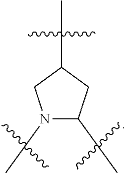 [SMILES: *C1CC(*)N(*)C1]

In some embodiments, [symbol] has a structure of

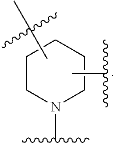

In one embodiment, [symbol] has a structure of

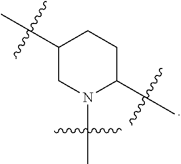 [SMILES: *C1CCC(*)N(*)C1]

In one embodiment, [symbol] has a structure of

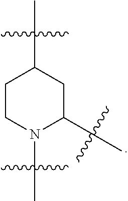 [SMILES: *C1CCN(*)C(*)C1]

In one embodiment, [symbol] has a structure of

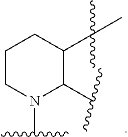 [SMILES: *C1CCCN(*)C1*]

In some embodiments, [symbol] has a structure of

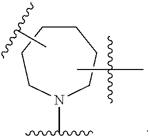

In one embodiment, [symbol] has a structure of

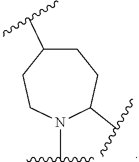 [SMILES: *C1CCC(*)N(*)CC1]

In some embodiments, [symbol] has a structure of

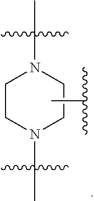

In one embodiment, [symbol] has a structure of

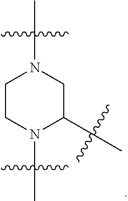 [SMILES: *C1CN(*)CCN1*]

In some embodiments, [symbol] has a structure of

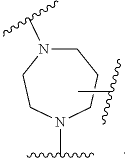

In one embodiment, [symbol] has a structure of

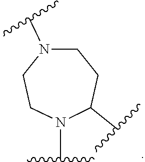 [SMILES: *C1CCN(*)CCN1*]

In some embodiments, [symbol] has a structure of

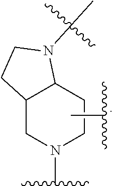

In one embodiment, [symbol] has a structure of

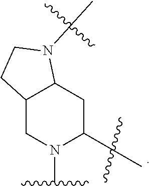 [SMILES: *C1CC2C(CCN2*)CN1*]

In some embodiments, [symbol] has a structure of

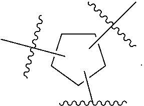

In one embodiment, [symbol] has a structure of

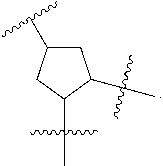 [SMILES: *C1CC(*)C(*)C1]

In some embodiments, the disclosure relates to ionizable lipids of Formula (LC-IIA) or (LC-IIA-2):

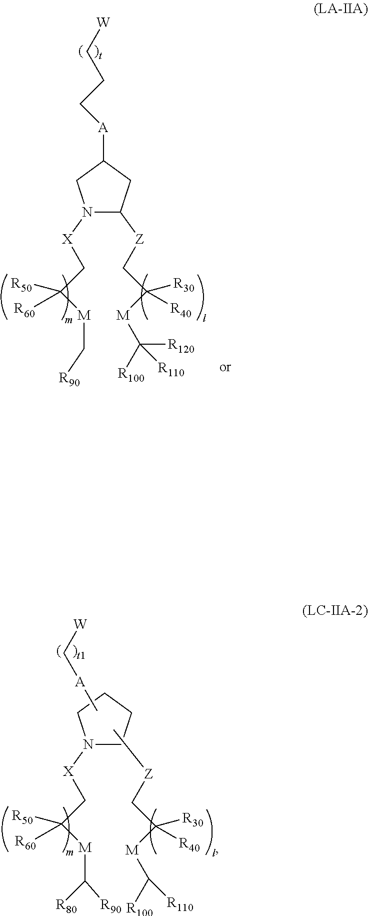

pharmaceutically acceptable salts thereof, and stereoisomers of any of the foregoing,
wherein:
   A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
   X is absent, —O—, —CO—, —N(R⁷)—, —O-alkylene-, -alkylene-O—, —OC(O)—, —C(O)O—, —NHC(O)—, —C(O)N(R⁷)—, or —S—;
   Z is absent, —O—, —N(R⁷)—, —O-alkylene-, -alkylene-O—, —OC(O)—, —C(O)O—, —NHC(O)—, —C(O)NH—, or —S—;
   each R⁷ is independently H, C₁-C₃ branched or unbranched alkyl, C₂-C₃ branched or unbranched alkenyl, cycloalkyl, hydroxyalkyl, or aminoalkyl;
   each M is independently M′,

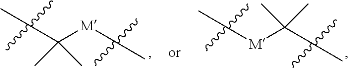

provided that at least one M in the formula is

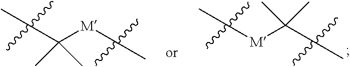

each M′ is independently a biodegradable moiety;
each of R₃₀, R₄₀, R₅₀, R₉₀, R₁₀₀, R₁₁₀, and R₁₂₀ is independently H, C₁-C₁₆ branched or unbranched alkyl, or C₁-C₁₆ branched or unbranched alkenyl, optionally interrupted with heteroatom or substituted with OH, SH, or halogen;
R₉₀ is C₁-C₁₅ branched or unbranched alkyl, C₁-C₁₅ branched or unbranched alkenyl, or cycloalkyl or substituted cycloalkyl;
t is 0, 1, 2, or 3;
l is an integer from 1 to 10;
m is an integer from 1 to 10; and
W is hydroxyl, or a bivalent heterocycle hydroxyalkyl, substituted or unsubstituted amino, substituted or unsubstituted aminocarbonyl, or substituted or unsubstituted heterocyclyl or heteroaryl, or one of the following moieties:

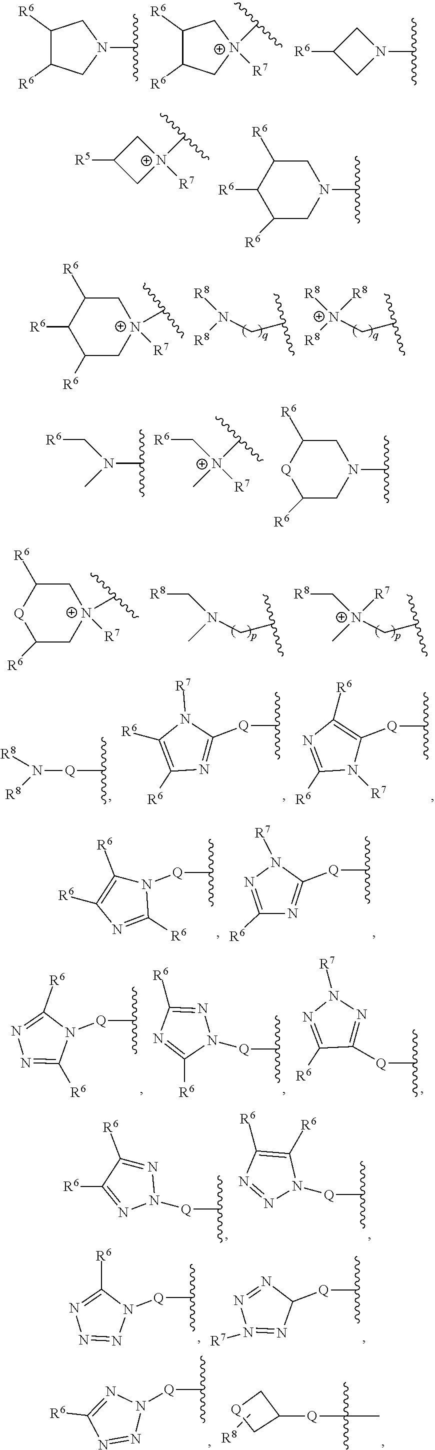

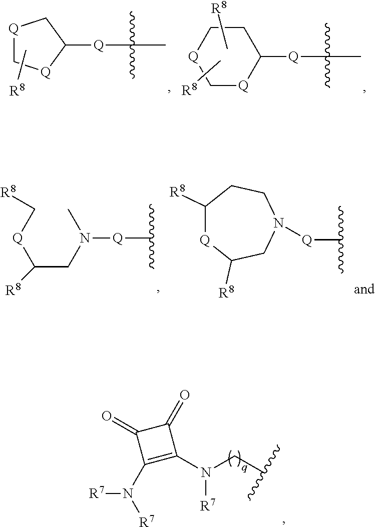

wherein
   each Q is independently absent, —O—, —C(O)—, —C(S)—, —C(O)O—, —(CH₂)_{q}—C(R⁷)₂, —C(O)N(R⁷)—, —C(S)N(R⁷)—, or —N(R⁷);
   R⁶ is independently H, alkyl, hydroxyl, hydroxyalkyl, alkoxy, —O-alkylene-O-alkyl, —O-alkylene-N(R⁷)₂, amino, alkylamino, aminoalkyl, N⁺(R⁷)₃-alkylene-Q-, thiol, or thiolalkyl;
   each R⁸ is independently H, alkyl, hydroxyalkyl, amino, aminoalkyl, alkylamino, heterocyclyl, heteroaryl, thiol, or thiolalkyl, or two R⁸ together with the nitrogen atom may form a ring, optionally substituted with one or more alkyl, hydroxy, hydroxyalkyl, alkoxy, alkylaminoalkyl, alkylamino, or aminoalkyl;
   q is 0, 1, 2, 3, 4, or 5; and
   p is 0, 1, 2, 3, 4, or 5.

In some embodiments, the disclosure relates to ionizable lipids of Formula (LC-IIIA):

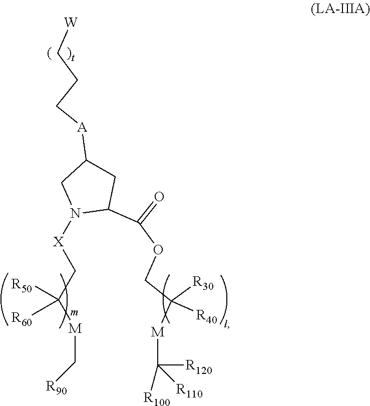

pharmaceutically acceptable salts thereof, and stereoisomers of any of the foregoing, wherein the definitions of the variables in (LC-IIIA) are the same as those in (LC-IIA).

In some embodiments, the disclosure relates to ionizable lipids of Formula (LC-IIB):

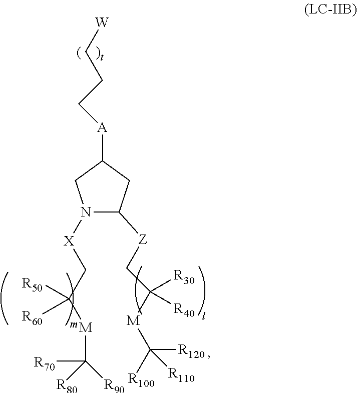

pharmaceutically acceptable salts thereof, and stereoisomers of any of the foregoing,
wherein:
   A is absent, —O—, —N(R⁷)—, —O-alkylene-, -alkylene-O—, —OC(O)—, —C(O)O—, —NHC(O)—, —C(O)N(R⁷)—, —N(R⁷)C(O)N(R⁷)—, —S—, —S—S—;
   X is absent, —O—, —CO—, —N(R⁷)—, —O-alkylene-, -alkylene-O—, —OC(O)—, —C(O)O—, —NHC(O)—, —C(O)N(R⁷)—, or —S—;
   Z is absent, —O—, —N(R⁷)—, —O-alkylene-, -alkylene-O—, —OC(O)—, —C(O)O—, —NHC(O)—, —C(O)NH—, or —S—;
   each R⁷ is independently H, C₁-C₃ branched or unbranched alkyl, C₂-C₃ branched or unbranched alkenyl, cycloalkyl, hydroxyalkyl, or aminoalkyl;
   each M is independently M′,

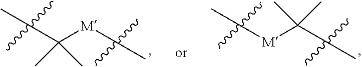

provided that at least one M in the formula is

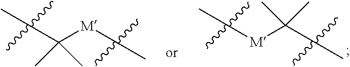

each M′ is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl, or C₁-C₁₆ branched or unbranched alkenyl, optionally interrupted with heteroatom or substituted with OH, SH, or halogen;
t is 0, 1, 2, or 3;
l is an integer from 1 to 10;
m is an integer from 1 to 10; and
W is hydroxyl, or a bivalent heterocycle hydroxyalkyl, substituted or unsubstituted amino, substituted or unsubstituted aminocarbonyl, or substituted or unsubstituted heterocyclyl or heteroaryl, or one of the following moieties:

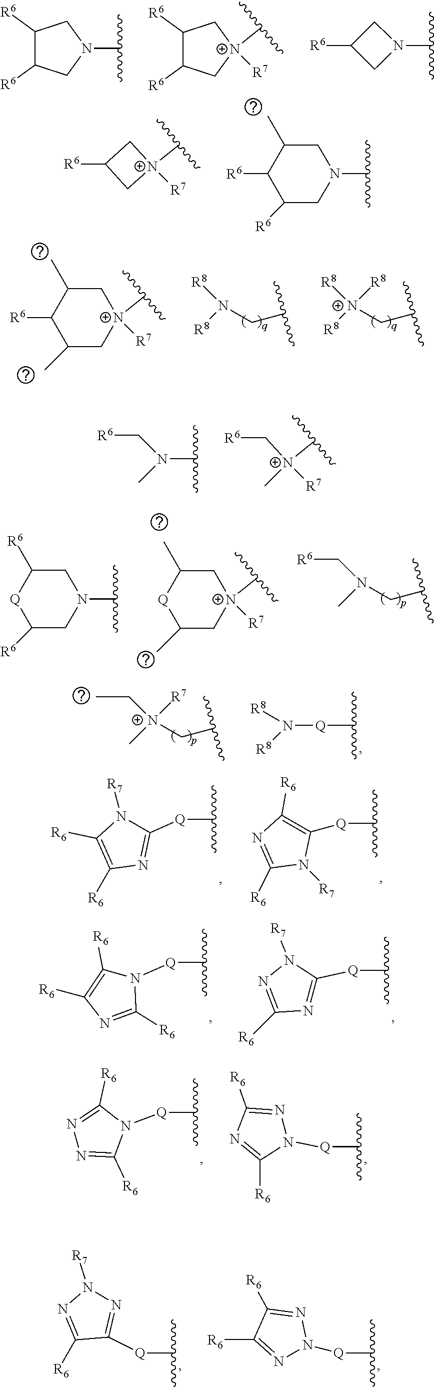

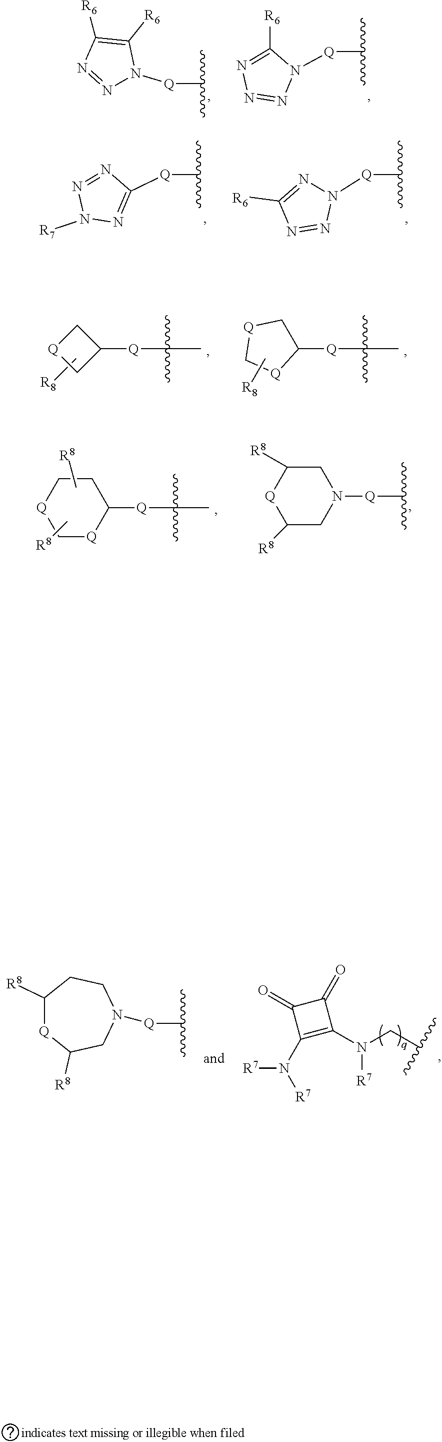

wherein
   each Q is independently absent, —O—, —C(O)—, —C(S)—, —C(O)O—, —(CH₂)_{q}—C(R⁷)₂, —C(O)N(R⁷)—, —C(S)N(R⁷)—, or —N(R⁷);
   R⁶ is independently H, alkyl, hydroxyl, hydroxyalkyl, alkoxy, —O-alkylene-O-alkyl, —O-alkylene-N(R⁷)₂, amino, alkylamino, aminoalkyl, N⁺(R⁷)₃-alkylene-Q-, thiol, or thiolalkyl;
   each R⁸ is independently H, alkyl, hydroxyalkyl, amino, aminoalkyl, alkylamino, heterocyclyl, heteroaryl, thiol, or thiolalkyl, or two R⁸ together with the nitrogen atom may form a ring, optionally substituted with one or more alkyl, hydroxy, hydroxyalkyl, alkoxy, alkylaminoalkyl, alkylamino, or aminoalkyl;
   q is 0, 1, 2, 3, 4, or 5; and
   p is 0, 1, 2, 3, 4, or 5.

In some embodiments, the disclosure relates to ionizable lipids of Formula (LC-IIIB):

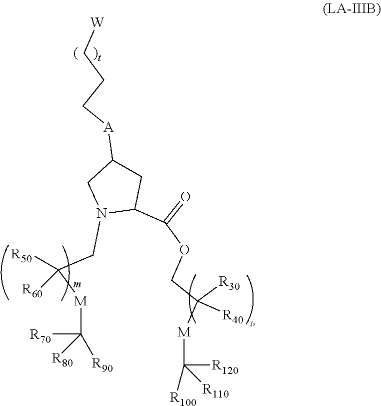

pharmaceutically acceptable salts thereof, and stereoisomers of any of the foregoing, wherein the definitions of the variables in (LC-IIIB) are the same as those in (LC-IIB).

In some embodiments, the disclosure relates to ionizable lipids of Formula (LC-IIC):

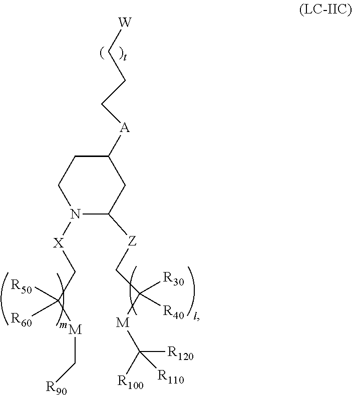

pharmaceutically acceptable salts thereof, and stereoisomers of any of the foregoing,
wherein:
   A is absent, —O—, —N(R⁷)—, —O-alkylene-, -alkylene-O—, —OC(O)—, —C(O)O—, —N(R⁷)C(O)—, —C(O)N(R′)—, N(R⁷)C(O)N(R⁷)—, —S—, —S—S—;
   each of R₃₀, R₄₀, R₅₀, R₆₀, R₁₀₀, R₁₁₀ and R₁₂₀ is independently H, C₁-C₁₆ branched or unbranched alkyl or C₁-C₁₆ branched or unbranched alkenyl, optionally interrupted with heteroatom or substituted with OH, SH, or halogen;
   R₉₀ is C₁-C₁₅ branched or unbranched alkyl, C₁-C₁₅ branched or unbranched alkenyl, cycloalkyl or substituted cycloalkyl;
   each R⁷ is independently H, C₁-C₃ branched or unbranched alkyl, C₂-C₃ branched or unbranched alkenyl, cycloalkyl, hydroxyalkyl, or aminoalkyl;
   each M is independently M′,

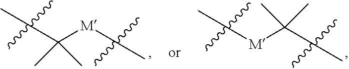

provided that at least one M in the formula is

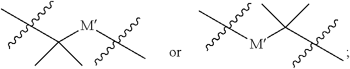

each M′ is independently a biodegradable moiety;
t is 0, 1, 2, or 3;
l is an integer from 1 to 10;
m is an integer from 1 to 10; and
W is hydroxyl, or a bivalent heterocycle hydroxyalkyl, substituted or unsubstituted amino, substituted or unsubstituted aminocarbonyl, or substituted or unsubstituted heterocyclyl or heteroaryl, or one of the following moieties:

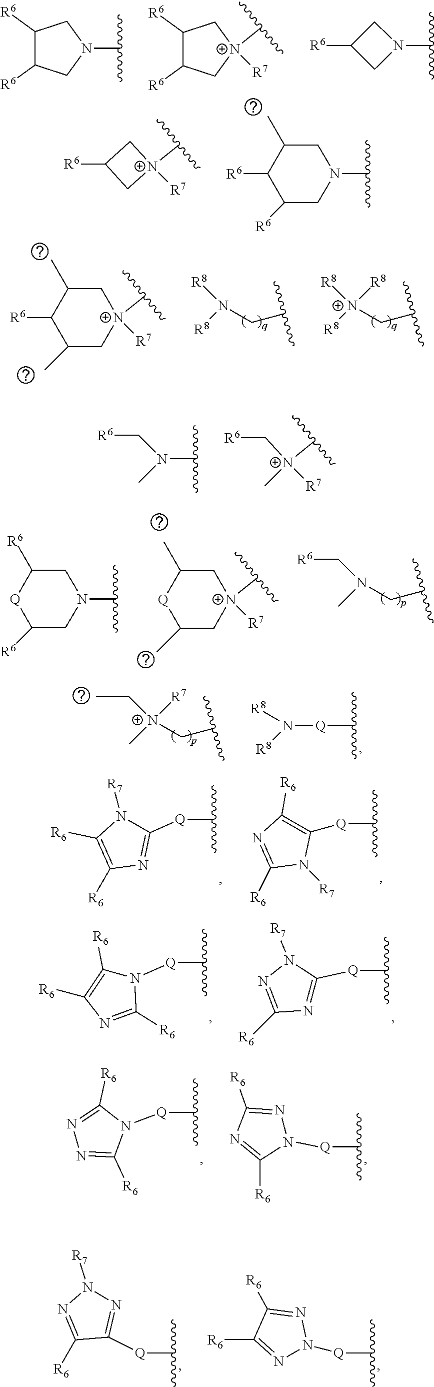

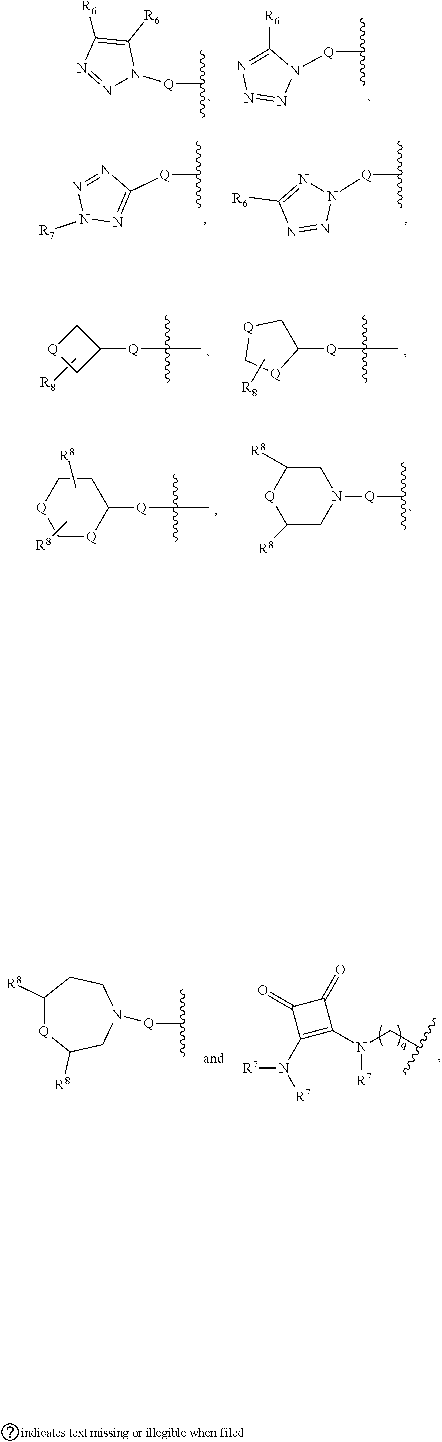

wherein
   each Q is independently absent, —O—, —C(O)—, —C(S)—, —C(O)O—, —(CH₂)_{q}—C(R⁷)₂, —C(O)N(R⁷)—, —C(S)N(R⁷)—, or —N(R⁷);
   R⁶ is independently H, alkyl, hydroxyl, hydroxyalkyl, alkoxy, —O-alkylene-O-alkyl, —O-alkylene-N(R⁷)₂, amino, alkylamino, aminoalkyl, N⁺(R⁷)₃-alkylene-Q-, thiol, or thiolalkyl;
   each R⁸ is independently H, alkyl, hydroxyalkyl, amino, aminoalkyl, alkylamino, heterocyclyl, heteroaryl, thiol, or thiolalkyl, or two R⁸ together with the nitrogen atom may form a ring, optionally substituted with one or more alkyl, hydroxy, hydroxyalkyl, alkoxy, alkylaminoalkyl, alkylamino, or aminoalkyl;
   q is 0, 1, 2, 3, 4, or 5; and
   p is 0, 1, 2, 3, 4, or 5.

In some embodiments, the disclosure relates to ionizable lipids of Formula (LC-IIIC) or:

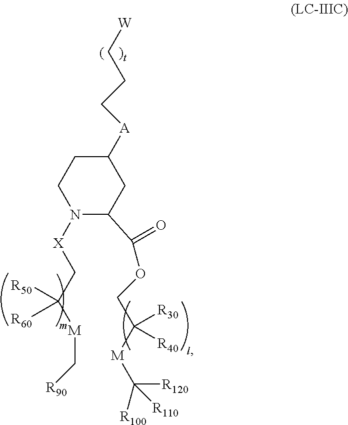

pharmaceutically acceptable salts thereof, and stereoisomers of any of the foregoing, wherein the definitions of the variables in (LC-IIIA) are the same as those in (LC-IIC).

In some embodiments, the disclosure relates to ionizable lipids of Formula (LC-IIID):

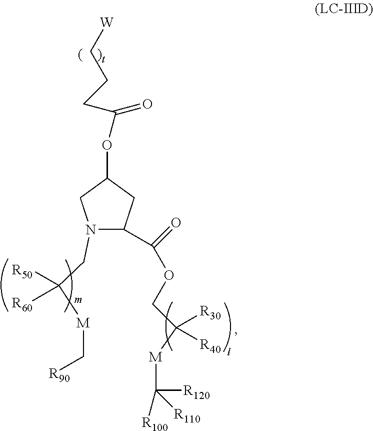

pharmaceutically acceptable salts thereof, and stereoisomers of any of the foregoing, wherein the definitions of the variables in (LC-IID) are the same as those defined above.

In some embodiments, the disclosure relates to ionizable lipids of Formula (LC-IIIE):

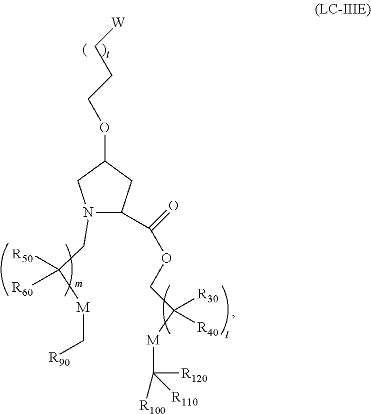

pharmaceutically acceptable salts thereof, and stereoisomers of any of the foregoing, wherein the definitions of the variables in (IID) are the same as those defined above.

Embodiments regarding various variables in the formulas (LC-IIA), (LC-IIB), (LC-IIC), (LC-IIIA), (LC-IIIB), (LC-IIIC), (LC-IIID), or (LC-IIIE) are further discussed below.

In some embodiments, X is absent, —O—, or —C(O)—. In one embodiment, X is absent. In one embodiment, X is —O—. In one embodiment, X is —C(O)—.

In some embodiments, Z is —O—, —C(O)O—, or —OC(O)—. In one embodiment, Z is —O—. In one embodiment, Z is —C(O)O— or —OC(O)—.

In some embodiments, each of R₃₀, R₄₀, R₅₀, and R₆₀ is H or C₁-C₄ branched or unbranched alkyl.

In some embodiments, each of R₃₀, R₄₀, R₅₀, and R₆₀ is H.

In some embodiments, each of R₇₀ and R₈₀ is H; and R₉₀ is C₁-C₁₅ branched or unbranched alkyl, C₁-C₁₅ branched or unbranched alkenyl. In some embodiments, R₉₀ is C₁-C₁₅ branched or unbranched alkyl. In some embodiments R₉₀ is C₁-C₁₂ branched or unbranched alkyl.

In some embodiments, R₇₀ is H; and each of R₈₀ and R₉₀ is independently C₁-C₁₅ branched or unbranched alkyl, C₁-C₁₅ branched or unbranched alkenyl. In some embodiments, each of R₈₀ and R₉₀ is independently C₁-C₁₅ branched or unbranched alkyl. In some embodiments, each of R₈₀ and R₉₀ is independently C₁-C₁₂ branched or unbranched alkyl. In some embodiments, each of R₈₀ and R₉₀ is independently C₁-C₈ branched or unbranched alkyl.

In some embodiments, R₁₀₀ is H; and each of R₁₁₀ and R₁₂₀ is independently C₁-C₁₅ branched or unbranched alkyl, C₁-C₁₅ branched or unbranched alkenyl. In some embodiments, each of R₁₁₀ and R₁₂₀ is independently C₁-C₁₅ branched or unbranched alkyl. In some embodiments, each of R₁₁₀ and R₁₂₀ is independently C₁-C₁₂ branched or unbranched alkyl. In some embodiments, each of R₁₁₀ and R₁₂₀ is independently C₁-C₈ branched or unbranched alkyl.

In some embodiments, 1 is from 3 to 10, from 3 to 7, or from 4 to 7.

In some embodiments, m is from 4 to 10, from 5 to 8, from 1 to 7, from 3 to 7, or from 1 to 5.

In some embodiments, 1 is 4, 5, 6, 7, 8, 9 or 10. In some embodiments, m is 4, 5, 6, 7, 8, 9 or 10.

In some embodiments, 1 is 4, 5, 6, or 7. In some embodiments, m is 3, 4, or 5. In some embodiments, m is 5, 6, 7, or 8.

In some embodiments, each M is

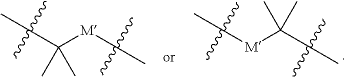

In some embodiments, M′ is is —OC(O)—, —C(O)O—, —N(R⁷)C(O)—, —C(O)N(R⁷)—, —C(O—R₁₃)—O—, —C(O)O(CH₂)ᵣ, —C(O)N(R⁷)(CH₂)ᵣ, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl, and r is 1, 2, 3, 4, or 5.

In some embodiments, at least one M in the formula is

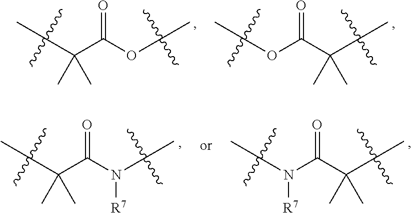

wherein R⁷ is H or methyl. In one embodiment, each M is

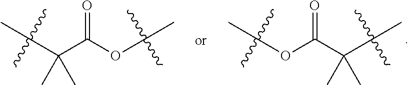

In one embodiment, each M is

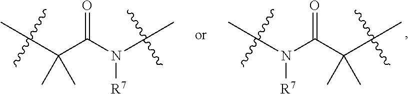

wherein R⁷ is H or methyl.

For all ionizable lipid formulas described above, embodiments regarding

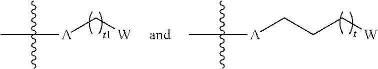

are further discussed below.

In some embodiments, A is absent, —O—, —N(R⁷)—, —C(O)N(R⁷)—, —N(R⁷)C(O)—, —OC(O)—, or —C(O)O—. In one embodiment, A is absent. In one embodiment, A is —O—. In one embodiment, A is —N(R⁷)—, wherein R⁷ is H or C₁-C₃ alkyl. In one embodiment, A is —OC(O)— or —C(O)O—. In one embodiment, A is —NHC(O)— or —C(O)NH—.

In some embodiments, t is 0, 1, or 2.

In some embodiments, W is OH.

In some embodiments, W is

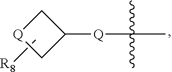

wherein Q is absent, —(CH₂)_{q}—C(R⁷)₂—, or —N(R⁷); q is 0 or 1; R⁷ is H or methyl; and each R⁸ is independently H or C₁-C₃ alkyl. In one embodiment, W is

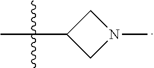 [SMILES: *C1CN(C)C1]

In some embodiments, W is

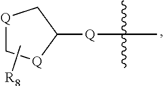

wherein Q is absent, —(CH₂)_{q}—C(R⁷)₂—, or —N(R⁷); q is 0 or 1; R⁷ is H or methyl; and each R⁸ is independently H or C₁-C₃ alkyl. In one embodiment, W is

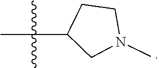 [SMILES: *C1CCN(C)C1]

In some embodiments, W is

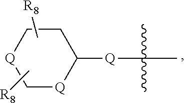

wherein Q is absent, —(CH₂)_{q}—C(R⁷)₂—, or —N(R⁷); q is 0 or 1; R⁷ is H or methyl; and each R⁸ is independently H or C₁-C₃ alkyl. In one embodiment, W is

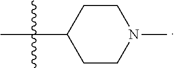 [SMILES: *C1CCN(C)CC1]

In some embodiments, W is

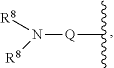

wherein Q is —(CH₂)_{q}—C(R⁷)₂—; q is 0 or 1; R⁷ is H or methyl; and each R⁸ is independently H or C₁-C₃ alkyl. In one embodiment, W is

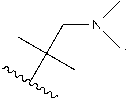 [SMILES: *C(C)(C)CN(C)C]

In one embodiment, W is

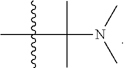 [SMILES: *C(C)(C)N(C)C]

In one embodiment, W is

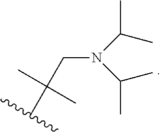 [SMILES: *C(C)(C)CN(C(C)C)C(C)C]

### In W is

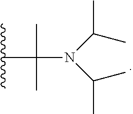 [SMILES: *C(C)(C)N(C(C)C)C(C)C]

In some embodiments, W is

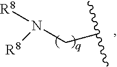

wherein q is 0, and each R⁸ is independently H, C₁-C₃ alkyl, hydroxyalkyl, heterocyclyl, or heteroaryl, optionally substituted with one or more alkyl. In one embodiment, W is

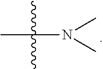 [SMILES: *N(C)C]

In one embodiment, W is

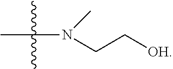 [SMILES: *N(C)CCO]

In one embodiment, W is

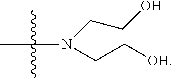 [SMILES: *N(CCO)CCO]

In one embodiment, W is

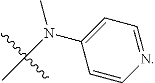 [SMILES: *N(C)c1ccncc1]

In one embodiment, W is

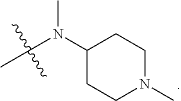 [SMILES: *N(C)C1CCN(C)CC1]

In one embodiment, W is

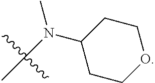 [SMILES: *N(C)C1CCOCC1]

In some embodiments, W is

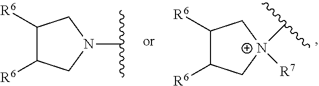

wherein each R⁶ is independently H, C₁-C₃ alkyl, hydroxyl, hydroxyalkyl, alkoxy, —O-alkylene-O-alkyl, or —O-alkylene-N(R⁷)₂ and each R⁷ is independently H or C₁-C₃ alkyl.

In one embodiment, W is

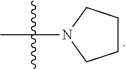 [SMILES: *N1CCCC1]

In one embodiment, W is

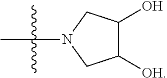 [SMILES: *N1CC(O)C(O)C1]

In one embodiment, W is

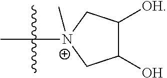 [SMILES: *[N+]1(C)CC(O)C(O)C1]

In one embodiment, W is

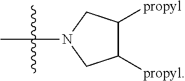

In one embodiment, W is

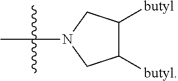

In one embodiment, W is

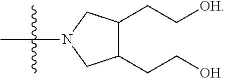 [SMILES: *N1CC(CCO)C(CCO)C1]

In one embodiment, W is

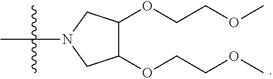 [SMILES: *N1CC(OCCOC)C(OCCOC)C1]

In one embodiment, W is

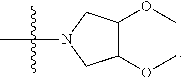 [SMILES: *N1CC(OC)C(OC)C1]

In one embodiment, W is

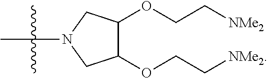 [SMILES: *N1CC(OCCN(C)C)C(OCCN(C)C)C1]

In one embodiment, W is

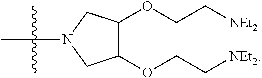

In one embodiment, W is

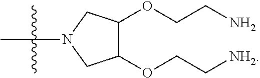 [SMILES: *N1CC(OCCN)C(OCCN)C1]

In some embodiments, W is

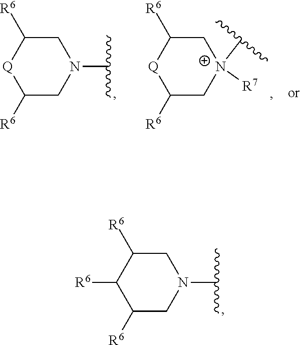

wherein each R⁶ is independently H, C₁-C₃ alkyl, hydroxyl, hydroxyalkyl, alkoxy, —O-alkylene-O-alkyl, or —O-alkylene-N(R⁷)₂; Q is —O—, —C(R⁷)₂—, or —N(R⁷); and R⁷ is H, C₁-C₃ alkyl, or hydroxyalkyl. In one embodiment, W is

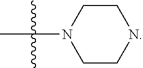

In one embodiment, W is

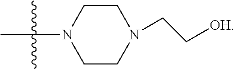 [SMILES: *N1CCN(CCO)CC1]

In one embodiment, W is

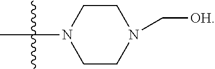 [SMILES: *N1CCN(CO)CC1]

In one embodiment, W is

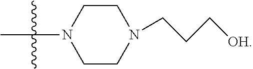 [SMILES: *N1CCN(CCCO)CC1]

In one embodiment, W is

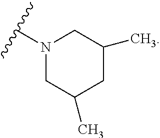 [SMILES: *N1CC(C)CC(C)C1]

In one embodiment, W is

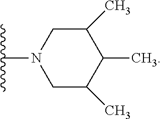 [SMILES: *N1CC(C)C(C)C(C)C1]

In one embodiment, W is

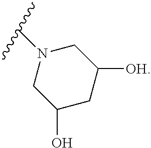 [SMILES: *N1CC(O)CC(O)C1]

In one embodiment, W is

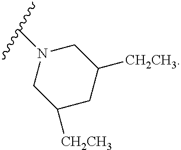 [SMILES: *N1CC(CC)CC(CC)C1]

In one embodiment, W is

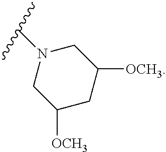 [SMILES: *N1CC(OC)CC(OC)C1]

In one embodiment, W is

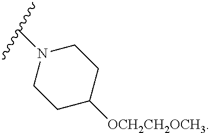

In one embodiment, W is

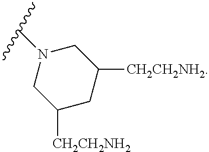

In one embodiment, W is

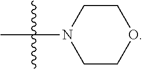 [SMILES: *N1CCOCC1]

In one embodiment, W is

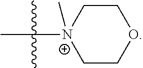 [SMILES: *[N+]1(C)CCOCC1]

In one embodiment, W is

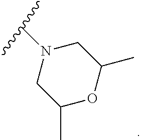 [SMILES: *N1CC(C)OC(C)C1]

In one embodiment, W is

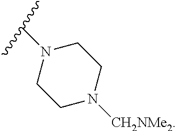

In one embodiment, W is

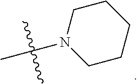 [SMILES: *N1CCCCC1]

In one embodiment, W is

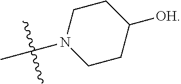 [SMILES: *N1CCC(O)CC1]

In one embodiment, W is

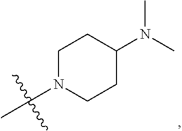 [SMILES: *N1CCC(N(C)C)CC1]

In some embodiments, W is

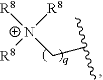

wherein q is 0, and each R⁸ is independently H, C₁-C₃ alkyl, or hydroxyalkyl. In one embodiment, W is

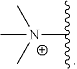 [SMILES: *[N+](C)(C)C]

In one embodiment, W is

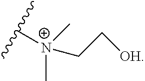 [SMILES: *[N+](C)(C)CCO]

In some embodiments, W is

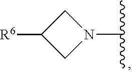

wherein R⁶ is independently H, alkyl, hydroxyl, hydroxyalkyl, alkoxy, —O-alkylene-O-alkyl, or —O-alkylene-N(R⁷)₂; and each R⁷ is independently H or C₁-C₃ alkyl. In one embodiment, W is

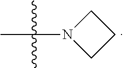 [SMILES: *N1CCC1]

In one embodiment, W is

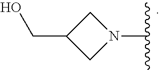 [SMILES: *N1CC(CO)C1]

In one embodiment, W is

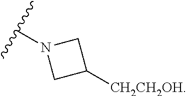

In one embodiment, W is

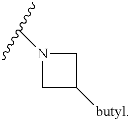

In one embodiment, W is

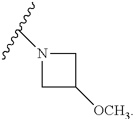 [SMILES: *N1CC(OC)C1]

In one embodiment, W is

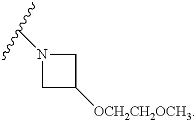

In one embodiment, W is

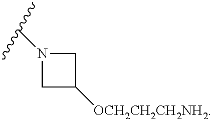

In some embodiments, W is

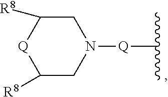

wherein each R⁸ is independently H, C₁-C₃ alkyl, or hydroxyalkyl; each Q is independently absent, —O—, —CO—, —C(R⁷)₂—, or —N(R⁷)—; and each R⁷ is independently H, C₁-C₃ alkyl, alkylamino, alkylaminoalkyl, or aminoalkyl. In one embodiment, W is

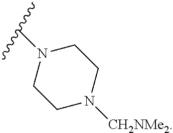

In one embodiment, W is

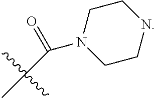

In some embodiments, W is

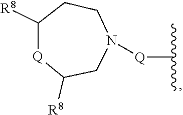

wherein each R⁸ is independently H, C₁-C₃ alkyl, or hydroxyalkyl; each Q is independently absent, —O—, —CO—, —C(R⁷)₂—, or —N(R⁷)—; and each R⁷ is independently H, C₁-C₃ alkyl, alkylamino, alkylaminoalkyl, or aminoalkyl. In one embodiment, W is

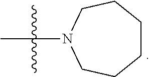 [SMILES: *N1CCCCCC1]

In one embodiment, W is

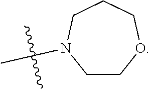 [SMILES: *N1CCCOCC1]

For all ionizable lipid formulas described above, embodiments regarding variables R₇₀, R₈₀, R₉₀, R₁₀₀, R₁₁₀, and R₁₂₀ are further discussed below.

In some embodiments, R₇₀ is H. In some embodiments, R₁₀₀ is H.

In these embodiments,

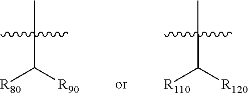

is independently selected from:

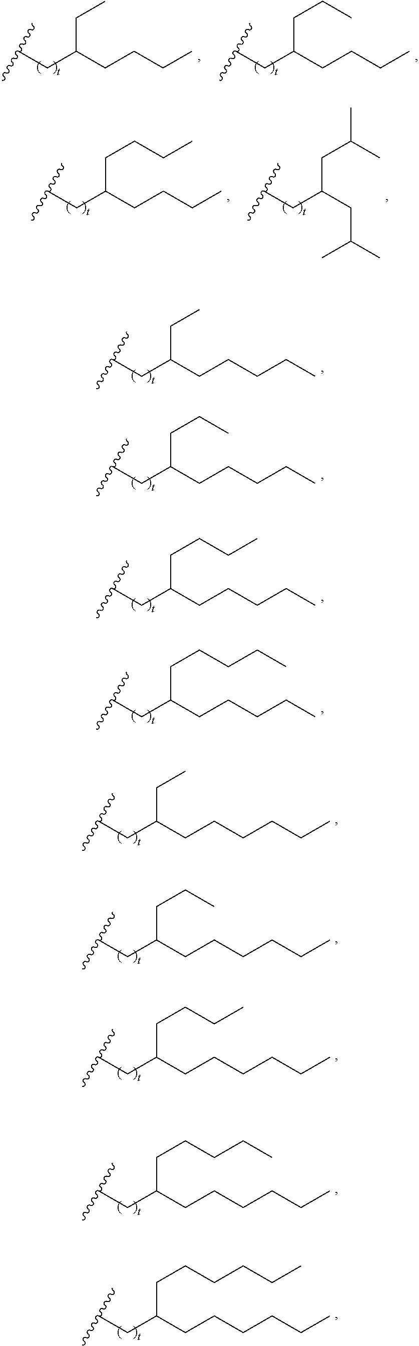

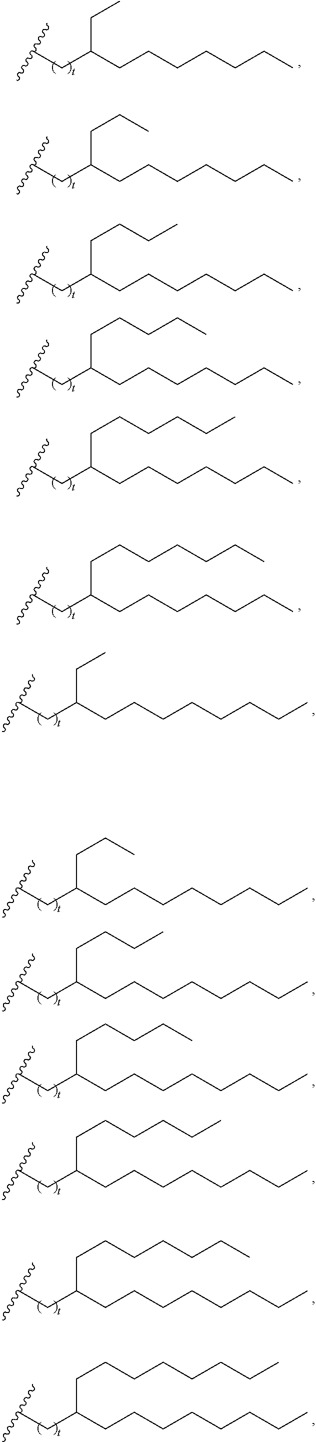

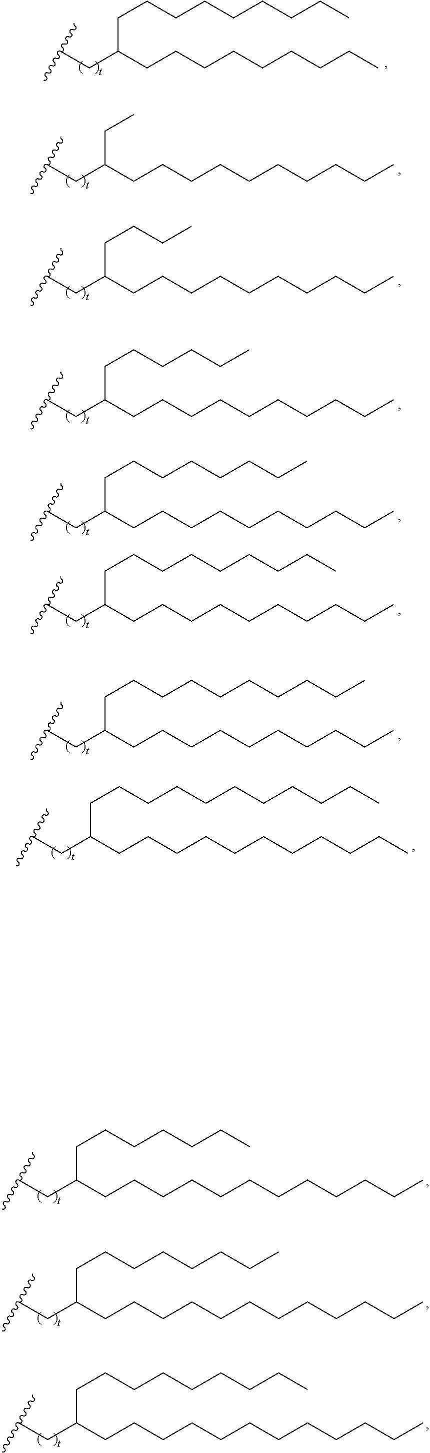

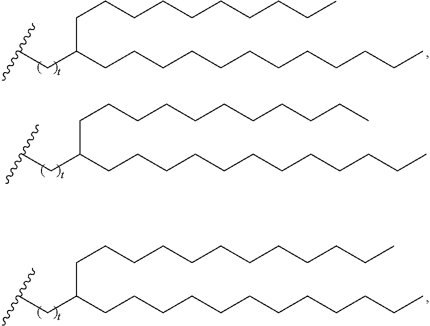

wherein t is 0, 1, 2, 3, 4, or 5.

In some embodiments, the pKa of the protonated form of the ionizable lipid compound described herein is about 4 to about 8, for instance, about 4.5 to about 8.0, about 4.6 to about 7.5, about 4.6 to about 7.1, about 4.6 to 5.5, about 4.8 to about 8.0, about 4.8 to about 7.5, about 4.8 to about 7.1, about 4.6 to 5.5, about 5.7 to about 6.5, about 5.7 to about 6.4, or from about 5.8 to about 6.2. In some embodiments, the pKa of the protonated form of the compound is about 5.5 to about 6.0. In some embodiments, the pKa of the protonated form of the compound is about 6.1 to about 6.3. In some embodiments, the pKa of the protonated form of the compound is about 4.7 to about 5.1. In some embodiments, the pKa of the protonated form of the compound is about 5.4 to about 7.1.

Non-limiting examples of ionizable lipid compounds disclosed here are set forth in Table 1 below.

TABLE 1
Exemplary ionizable lipid compounds.

| Lipid | Structure | IUPAC Name |
|---|---|---|
| 2243 | 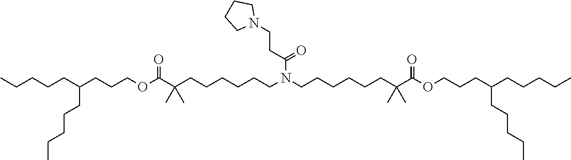 [SMILES: CCCCCC(CCCCC)CCCOC(=O)C(C)(C)CCCCCCN(CCCCCCC(C)(C)C(=O)OCCCC(CCCCC)CCCCC)C(=O)CCN1CCCC1] | bis(4-pentylnonyl) 8,8′-((3-(pyrrolidin-1- yl)propanoyl)azanediyl) bis(2,2- dimethyloctanoate) |
| 2330 | 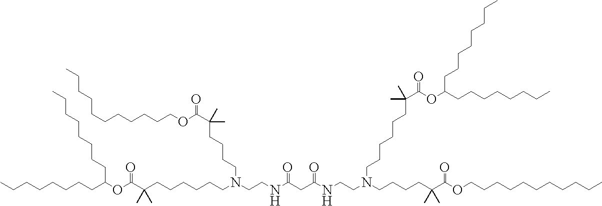 [SMILES: CCCCCCCCCCCOC(=O)C(C)(C)CCCCN(CCCCCCC(C)(C)C(=O)OC(CCCCCCCC)CCCCCCCC)CCNC(=O)CC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,19-bis(5,5- dimethyl-6-oxo-6- (undecyloxy)hexyl)- 2,2,26,26- tetramethyl-13,15- dioxo-9,12,16,19- tetraazaheptacosanedioate |
| 2331 | 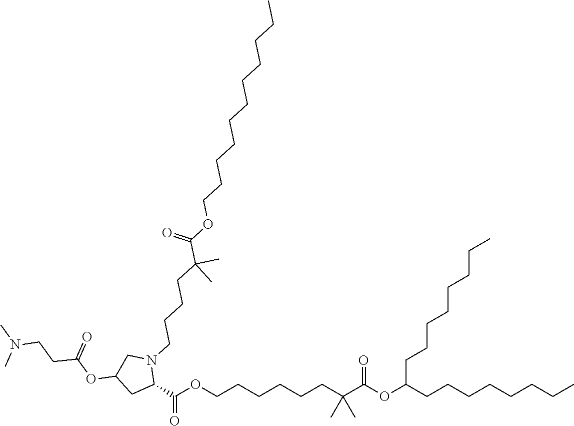 [SMILES: CCCCCCCCCCCOC(=O)C(C)(C)CCCCN1CC(OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl- 8-oxooctyl (2S)-1- (5,5-dimethyl-6-oxo- 6- (undecyloxy)hexyl)- 4-((3- (dimethylamino) propanoyl) oxy)pyrrolidine- 2-carboxylate |
| 2333 | 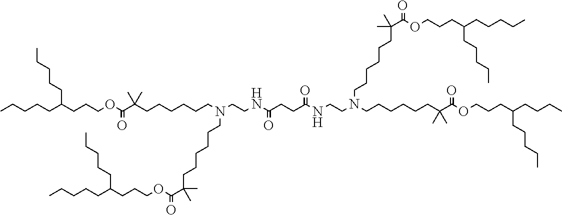 [SMILES: CCCCCC(CCCC)CCCOC(=O)C(C)(C)CCCCCCN(CCCCCCC(C)(C)C(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] | bis(4-pentylnonyl) 9,20-bis(7,7- dimethyl-8-oxo-8- ((4- pentylnonyl)oxy)octyl)- 2,2,27,27- tetramethyl-13,16- dioxo-9,12,17,20- tetraazaoctacosanedioate |
| 2335 | 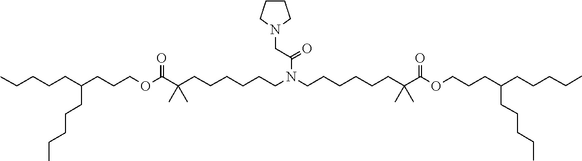 [SMILES: CCCCCC(CCCCC)CCCOC(=O)C(C)(C)CCCCCCN(CCCCCCC(C)(C)C(=O)OCCCC(CCCCC)CCCCC)C(=O)CN1CCCC1] | bis(4-pentylnonyl) 8,8′-((2-(pyrrolidin-1- yl)acetyl)azanediyl)bis (2,2- dimethyloctanoate) |
| 2365 | 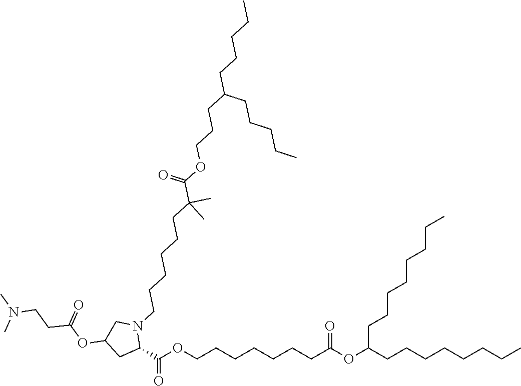 [SMILES: CCCCCCCCC(CCCCCCCC)OC(=O)CCCCCCCOC(=O)[C@@H]1CC(OC(=O)CCN(C)C)CN1CCCCCCC(C)(C)C(=O)OCCCC(CCCCC)CCCCC] | 8-(heptadecan-9- yloxy)-8-oxooctyl (2S)-1-(7,7-dimethyl- 8-oxo-8-((4- pentylnonyl)oxy)octyl)- 4-((3- (dimethylamino) propanoyl) oxy)pyrrolidine- 2-carboxylate |
| 2366 | 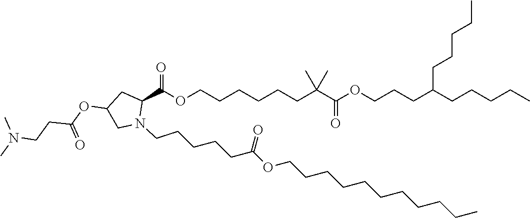 [SMILES: CCCCCCCCCCCOC(=O)CCCCCN1CC(OC(=O)CCN(C)C)C[C@H]1C(=O)OCCCCCCC(C)(C)C(=O)OCCCC(CCCCC)CCCCC] | 7,7-dimethyl-8-oxo- 8-((4- pentylnonyl)oxy)octyl (2S)-4-((3- (dimethylamino) propanoyl)oxy)- 1-(6-oxo-6- (undecyloxy)hexyl) pyrrolidine-2- carboxylate |
| 2367 | 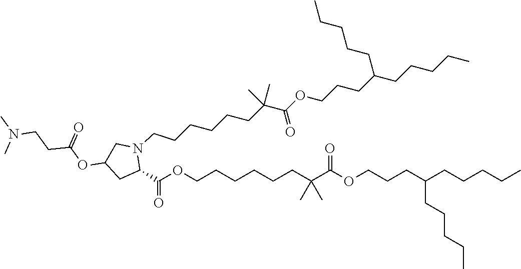 [SMILES: CCCCCC(CCCCC)CCCOC(=O)C(C)(C)CCCCCCOC(=O)[C@@H]1CC(OC(=O)CCN(C)C)CN1CCCCCCC(C)(C)C(=O)OCCCC(CCCCC)CCCCC] | 7,7-dimethyl-8-oxo- 8-((4- pentylnonyl)oxy)octyl (2S)-1-(7,7- dimethyl-8-oxo-8- ((4- pentylnonyl)oxy) octyl)-4-((3- (dimethylamino) propanoyl) oxy)pyrrolidine- 2-carboxylate |
| 2368 | 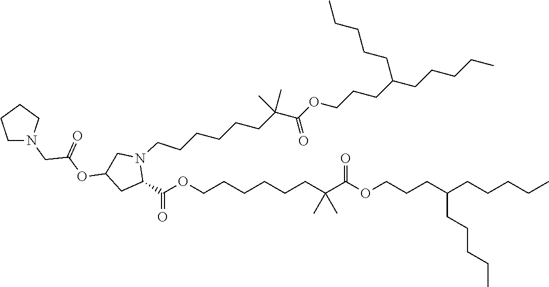 [SMILES: CCCCCC(CCCCC)CCCOC(=O)C(C)(C)CCCCCCOC(=O)[C@@H]1CC(OC(=O)CN2CCCC2)CN1CCCCCCC(C)(C)C(=O)OCCCC(CCCCC)CCCCC] | 7,7-dimethyl-8-oxo- 8-((4- pentylnonyl)oxy) octyl (2S)-1-(7,7- dimethyl-8-oxo-8- ((4- pentylnonyl)oxy)octyl)- 4-(2-(pyrrolidin-1- yl)acetoxy)pyrrolidine- 2-carboxylate |
| 2369 | 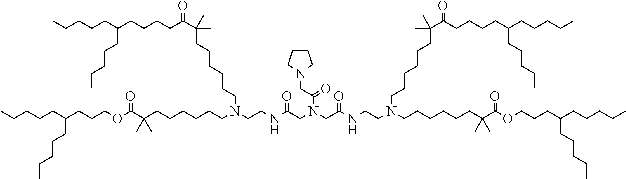 [SMILES: CCCCCC(CCCCC)CCCCC(=O)C(C)(C)CCCCCCN(CCCCCCC(C)(C)C(=O)OCCCC(CCCCC)CCCCC)CCNC(=O)CN(CC(=O)NCCN(CCCCCCC(C)(C)C(=O)CCCCC(CCCCC)CCCCC)CCCCCCC(C)(C)C(=O)OCCCC(CCCCC)CCCCC)C(=O)CN1CCCC1] | bis(4-pentylnonyl) 9,21-bis(7,7- dimethyl-8-oxo-8-((4- pentylnonyl)oxy)octyl)- 2,2,28,28- tetramethyl-13,17- dioxo-15-(2- (pyrrolidin-1- yl)acetyl)- 9,12,15,18,21- pentaazanonacosanedioate |
| 2370 | 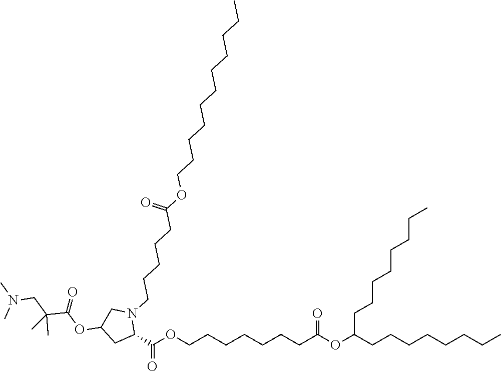 [SMILES: CCCCCCCCCCCOC(=O)CCCCCN1CC(OC(=O)C(C)(C)CN(C)C)C[C@H]1C(=O)OCCCCCCCC(=O)OC(CCCCCCCC)CCCCCCCC] | 8-(heptadecan-9- yloxy)-8-oxooctyl (2S)-4-((3- (dimethylamino)-2,2- dimethylpropanoyl) oxy)-1-(6-oxo-6- (undecyloxy)hexyl) pyrrolidine-2- carboxylate |
| 2383 | 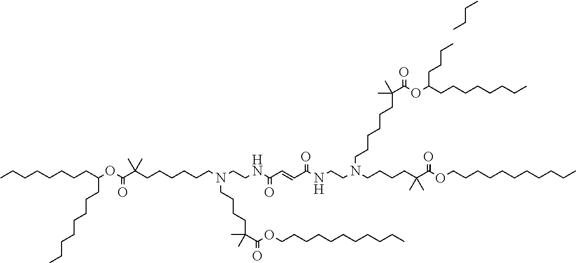 | di(heptadecan-9-yl) (E)-9,20-bis(5,5- dimethyl-6-oxo-6- (undecyloxy)hexyl)- 2,2,27,27- tetramethyl-13,16- dioxo-9,12,17,20- tetraazaoctacos-14- enedioate |
| 2392 | 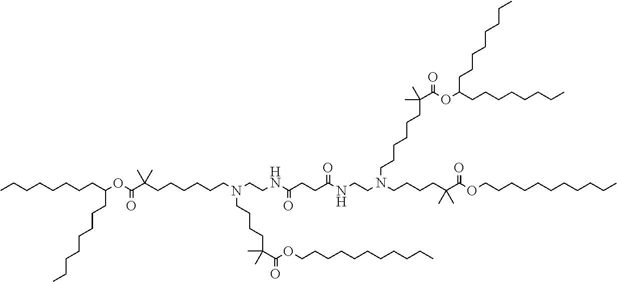 [SMILES: CCCCCCCCCCCOC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | di(heptadecan-9-yl) 9,20-bis(5,5- dimethyl-6-oxo-6- (undecyloxy)hexyl)- 2,2,27,27- tetramethyl-13,16- dioxo-9,12,17,20- tetraazaoctacosanedioate |
| 2398 | 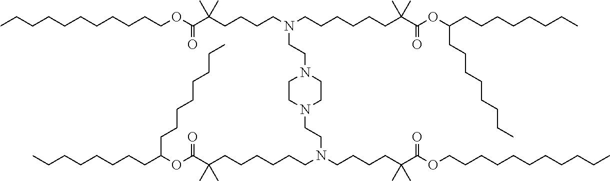 | di(heptadecan-9-yl) 8,8′-((piperazine-1,4- diylbis(ethane-2,1- diyl))bis((5,5- dimethyl-6-oxo-6- (undecyloxy)hexyl) azanediyl))bis(2,2- dimethyloctanoate) |
| 2424 | 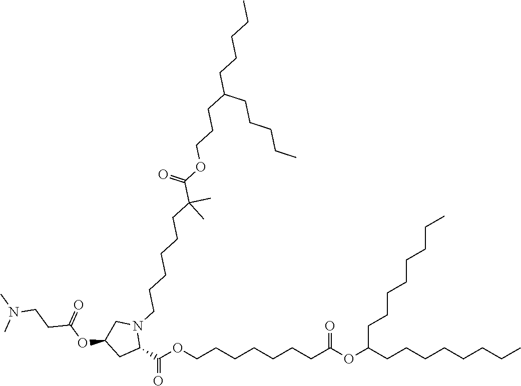 [SMILES: CCCCCCCCC(CCCCCCCC)OC(=O)CCCCCCCOC(=O)[C@@H]1C[C@@H](OC(=O)CCN(C)C)CN1CCCCCCC(C)(C)C(=O)OCCCC(CCCCC)CCCCC] | 8-(heptadecan-9- yloxy)-8-oxooctyl (2S,4R)-1-(7,7- dimethyl-8-oxo-8- ((4- pentylnonyl)oxy)octyl)- 4-((3- (dimethylamino) propanoyl) oxy)pyrrolidine- 2-carboxylatc |
| 2425 | 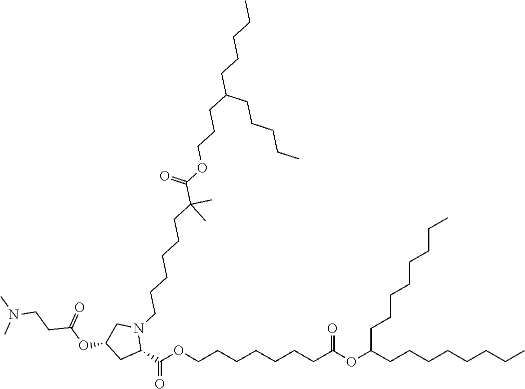 [SMILES: CCCCCCCCC(CCCCCCCC)OC(=O)CCCCCCCOC(=O)[C@@H]1C[C@H](OC(=O)CCN(C)C)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy)octyl)- 4-((3- (dimethylamino) propanoyl) oxy)pyrrolidine- 2-carboxylate |
| 2432 | 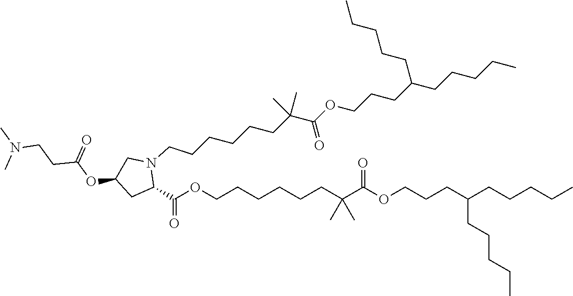 [SMILES: CCCCCC(CCCCC)CCCOC(=O)C(C)(C)CCCCCCOC(=O)[C@@H]1C[C@@H](OC(=O)CCN(C)C)CN1CCCCCCC(C)(C)C(=O)OCCCC(CCCCC)CCCCC] | 7,7-dimethyl-8-oxo- 8-((4- pentylnonyl)oxy)octyl (2S,4R)-1-(7,7- dimethyl-8-oxo-8- ((4- pentylnonyl)oxy)octyl)- 4-((3- (dimethylamino) propanoyl) oxy)pyrrolidine- 2-carboxylate |
| 2433 | 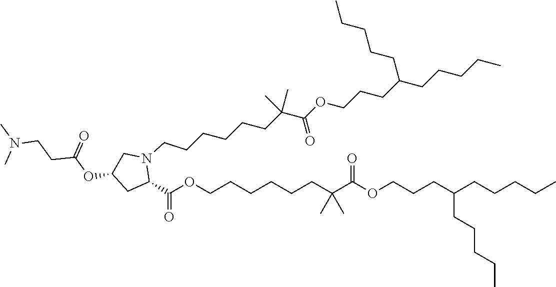 [SMILES: CCCCCC(CCCCC)CCCOC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | 7,7-dimethyl-8-oxo- 8-((4- pentylnonyl)oxy)octyl (2S,4S)-1-(7,7- dimethyl-8-oxo-8-((4- pentylnonyl)oxy)octyl)- 4-((3- (dimethylamino) propanoyl) oxy)pyrrolidine- 2-carboxylate |
| 2441 | 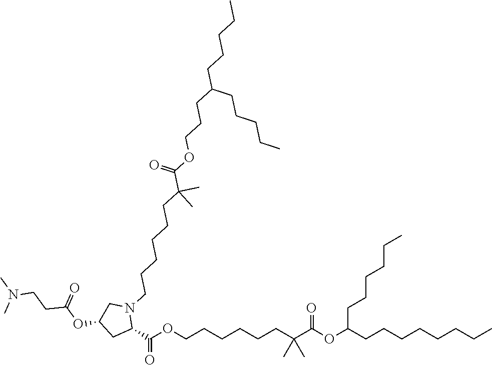 [SMILES: CCCCCCCCC(CCCCCC)OC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | 7,7-dimethyl-8-oxo- 8-(pentadecan-7- yloxy)octyl (2S,4S)- 1-(7,7-dimethyl-8- oxo-8-((4- pentylnonyl)oxy)octyl)- 4-((3- (dimethylamino) propanoyl) oxy)pyrrolidine- 2-carboxylatc |
| 2442 | 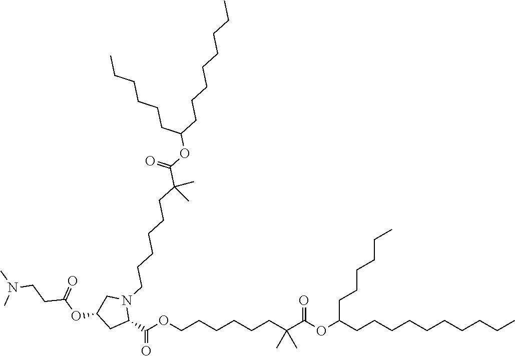 [SMILES: CCCCCCCCCCC(CCCCCC)OC(=O)C(C)(C)CCCCCCOC(=O)[C@@H]1C[C@H](OC(=O)CCN(C)C)CN1CCCCCCC(C)(C)C(=O)OC(CCCCCC)CCCCCCCC] | 7,7-dimethyl-8-oxo- 8-(pentadecan-7- yloxy)octyl (2S,4S)- 1-(7,7-dimethyl-8- oxo-8-(pentadecan-7- yloxy)octyl)-4-((3- (dimethylamino) propanoyl)oxy) pyrrolidine- 2-carboxylate |
| 2443 | 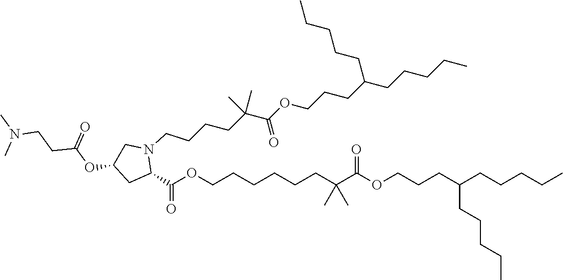 [SMILES: CCCCCC(CCCCC)CCCOC(=O)C(C)(C)CCCCCCOC(=O)[C@@H]1C[C@H](OC(=O)CCN(C)C)CN1CCCCC(C)(C)C(=O)OCCCC(CCCCC)CCCCC] | 7,7-dimethyl-8-oxo- 8-((4- pentylnonyl)oxy)octyl (2S,4S)-1-(5,5- dimethyl-6-oxo-6- ((4- pentylnonyl)oxy) hexyl)-4-((3- (dimethylamino) propanoyl) oxy)pyrrolidine- 2-carboxylate |
| 2454 | 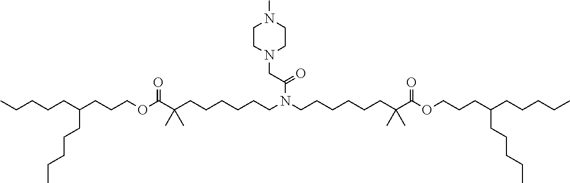 [SMILES: CCCCCC(CCCCC)CCCOC(=O)C(C)(C)CCCCCCN(CCCCCCC(C)(C)C(=O)OCCCC(CCCCC)CCCCC)C(=O)CN1CCN(C)CC1] | bis(4-pentylnonyl) 8,8′-((2-(4- methylpiperazin-1- yl)acetyl)azanediyl) bis(2,2- dimethyloctanoate) |
| 2481 | 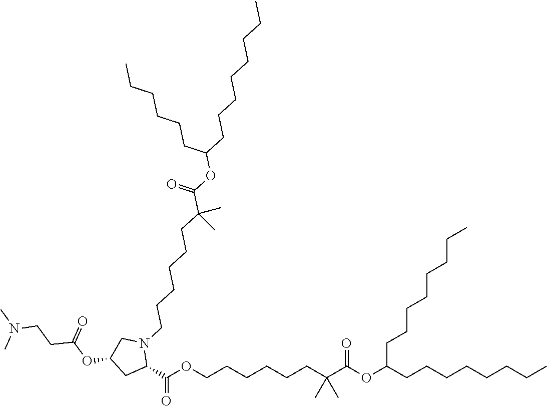 [SMILES: CCCCCCCCC(CCCCCC)OC(=O)C(C)(C)CCCCCCN1C[C@@H](OC(=O)CCN(C)C)C[C@H]1C(=O)OCCCCCCC(C)(C)C(=O)OC(CCCCCCCC)CCCCCCCC] | 8-(heptadecan-9- yloxy)-7,7-dimethyl- 8-oxooctyl (2S,4S)-1- (7,7-dimethyl-8-oxo- 8-(pentadecan-7- yloxy)octyl)-4-((3- (dimethylamino) propanoyl)oxy) pyrrolidine- 2-carboxylate |
| 2482 | 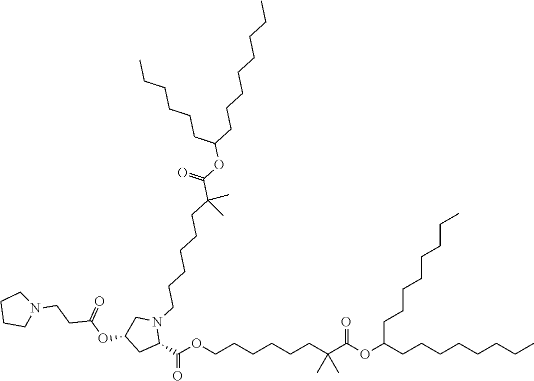 [SMILES: CCCCCCCCC(CCCCCC)OC(=O)C(C)(C)CCCCCCN1C[C@@H](OC(=O)CCN2CCCC2)C[C@H]1C(=O)OCCCCCCC(C)(C)C(=O)OC(CCCCCCCC)CCCCCCCC] | 8-(heptadecan-9- yloxy)-7,7-dimethyl- 8-oxooctyl (2S,4S)-1- (7,7-dimethyl-8-oxo- 8-(pentadecan-7- yloxy)octyl)-4-((3- (pyrrolidin-1- yl)propanoyl)oxy) pyrrolidine-2- carboxylate |
| 2483 | 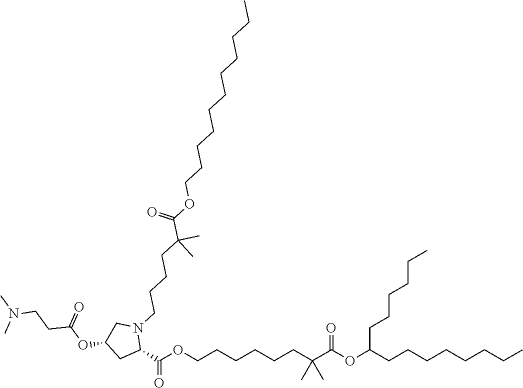 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCC)CCCCCCCC] | 7,7-dimethyl-8-oxo- 8-(pentadecan-7- yloxy)octyl (2S,4S)- 1-(5,5-dimethyl-6- oxo-6- (undecyloxy)hexyl)- 4-((3- (dimethylamino) propanoyl) oxy)pyrrolidine- 2-carboxylate |
| 2485 | 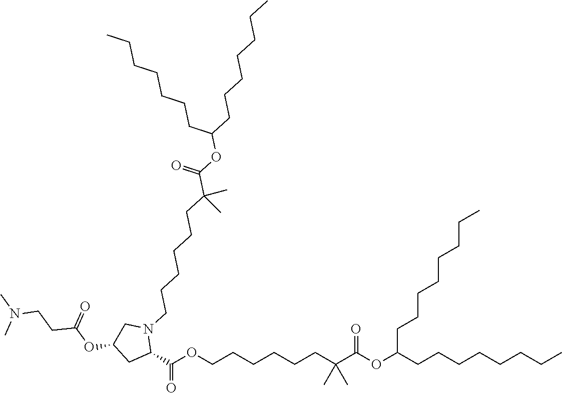 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OC(CCCCCCC)CCCCCCC] | 8-(heptadecan-9- yloxy)-7,7-dimethyl- 8-oxooctyl (2S,4S)-1- (7,7-dimethyl-8-oxo- 8-(pentadecan-8- yloxy)octyl)-4-((3- (dimethylamino) propanoyl) oxy)pyrrolidine- 2-carboxylate |
| 2486 | 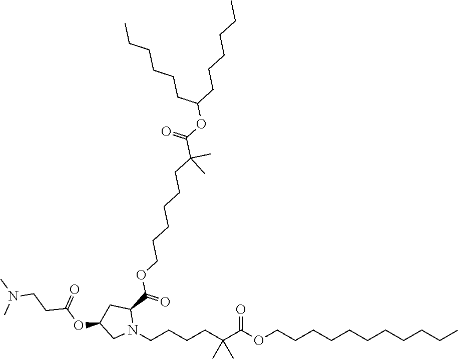 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCC)CCCCCC] | 7,7-dimethyl-8-oxo- 8-(tridecan-7- yloxy)octyl (2S,4S)- 1-(5,5-dimethyl-6- oxo-6- (undecyloxy)hexyl)- 4-((3- (dimethylamino) propanoyl)oxy) pyrrolidine- 2-carboxylatc |
| 2487 | 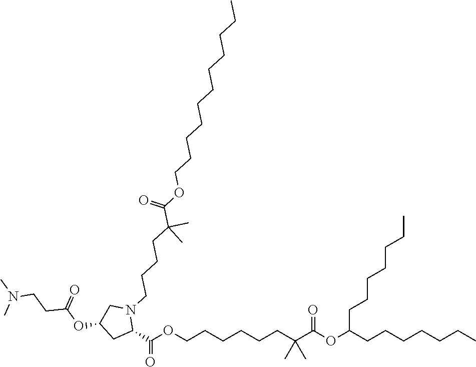 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCCC)CCCCCCC] | 7,7-dimethyl-8-oxo- 8-(pentadecan-8- yloxy)octyl (2S,4S)- 1-(5,5-dimethyl-6- oxo-6- (undecyloxy)hexyl)- 4-((3- (dimethylamino) propanoyl) oxy)pyrrolidine- 2-carboxylate |
| 2488 | 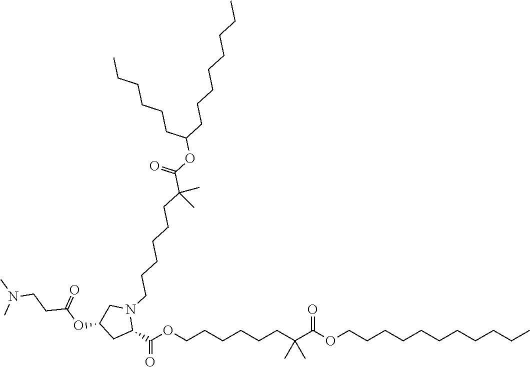 [SMILES: CCCCCCCCCCCOC(=O)C(C)(C)CCCCCCOC(=O)[C@@H]1C[C@H](OC(=O)CCN(C)C)CN1CCCCCCC(C)(C)C(=O)OC(CCCCCC)CCCCCCCC] | 7,7-dimethyl-8-oxo- 8-(undecyloxy)octyl (2S,4S)-1-(7,7- dimethyl-8-oxo-8- (pentadecan-7- yloxy)octyl)-4-((3- (dimethylamino) propanoyl) oxy)pyrrolidine- 2-carboxylate |
| 2498 | 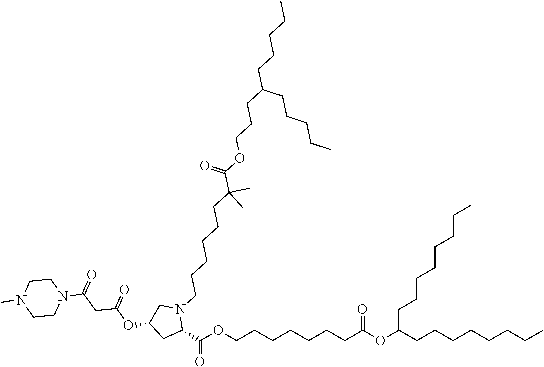 [SMILES: CCCCCCCCC(CCCCCCCC)OC(=O)CCCCCCCOC(=O)[C@@H]1C[C@H](OC(=O)CC(=O)N2CCN(C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3-(4- methylpiperazin-1- yl)-3- oxopropanoyl)oxy) pyrrolidine-2- carboxylate |
| 2499 | 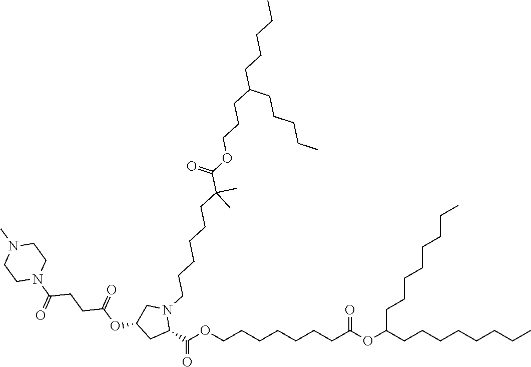 [SMILES: CCCCCCCCC(CCCCCCCC)OC(=O)CCCCCCCOC(=O)[C@@H]1C[C@H](OC(=O)CCC(=O)N2CCN(C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((4-(4- methylpiperazin-1- yl)-4- oxobutanoyl)oxy) pyrrolidine-2- carboxylate |
| 2500 | 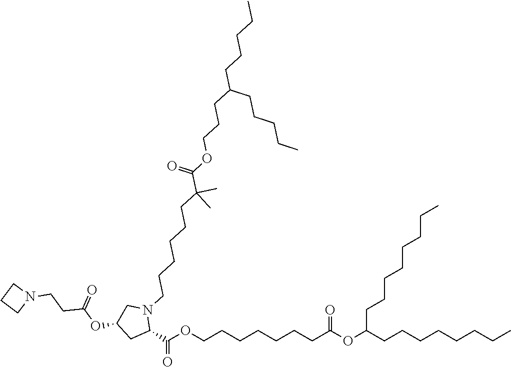 [SMILES: CCCCCCCCC(CCCCCCCC)OC(=O)CCCCCCCOC(=O)[C@@H]1C[C@H](OC(=O)CCN2CCC2)CN1CCCCCCC(C)(C)C(=O)OCCCC(CCCCC)CCCCC] | 8-(heptadecan-9- yloxy)-8-oxooctyl (2S,4S)-4-((3- (azetidin-1- yl)propanoyl)oxy)-1- (7,7-dimethyl-8-oxo- 8-((4- pcntylnonyl)oxy)octyl) pyrrolidine-2- carboxylate |
| 2501 | 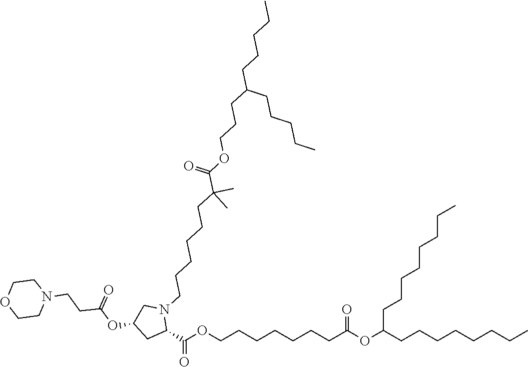 [SMILES: CCCCCCCCC(CCCCCCCC)OC(=O)CCCCCCCOC(=O)[C@@H]1C[C@H](OC(=O)CCN2CCOCC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy)octyl)- 4-((3- morpholinopropanoyl) oxy)pyrrolidine-2- carboxylate |
| 2502 | 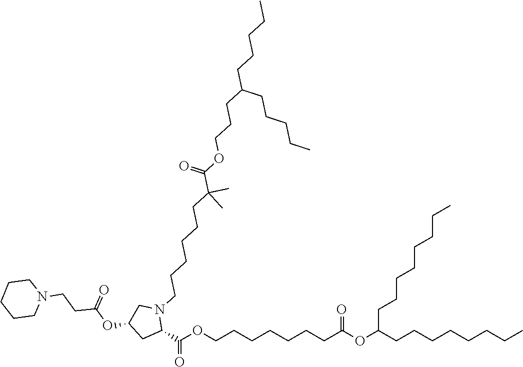 [SMILES: CCCCCCCCC(CCCCCCCC)OC(=O)CCCCCCCOC(=O)[C@@H]1C[C@H](OC(=O)CCN2CCCCC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy)octyl)- 4-((3-(piperidin-1- yl)propanoyl)oxy) pyrrolidine-2- carboxylate |
| 2503 | 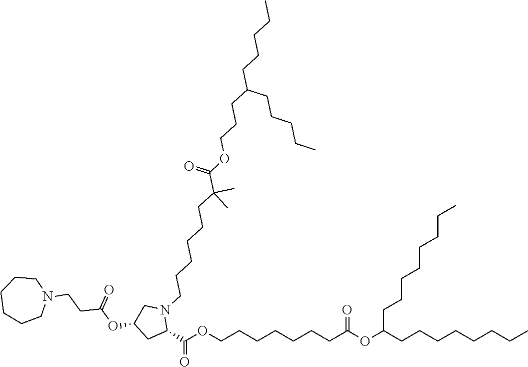 [SMILES: CCCCCCCCC(CCCCCCCC)OC(=O)CCCCCCCOC(=O)[C@@H]1C[C@H](OC(=O)CCN2CCCCCC2)CN1CCCCCCC(C)(C)C(=O)OCCCC(CCCCC)CCCCC] | 8-(heptadecan-9- yloxy)-8-oxooctyl (2S,4S)-4-((3- (azepan-1- yl)propanoyl)oxy)-1- (7,7-dimethyl-8-oxo- 8-((4- pentylnonyl)oxy)octyl) pyrrolidine-2- carboxylate |
| 2504 | 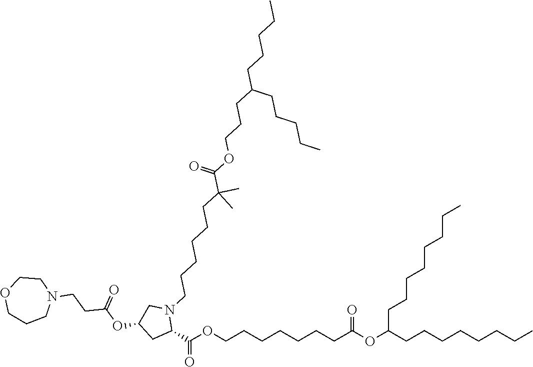 [SMILES: CCCCCCCCC(CCCCCCCC)OC(=O)CCCCCCCOC(=O)[C@@H]1C[C@H](OC(=O)CCN2CCCOCC2)CN1CCCCCCC(C)(C)C(=O)OCCCC(CCCCC)CCCCC] | 8-(hcptadccan-9- yloxy)-8-oxooctyl (2S,4S)-4-((3-(1,4- oxazepan-4- yl)propanoyl)oxy)-1- (7,7-dimethyl-8-oxo- 8-((4- pentylnonyl)oxy) octyl)pyrrolidine-2- carboxylate |
| 2505 | 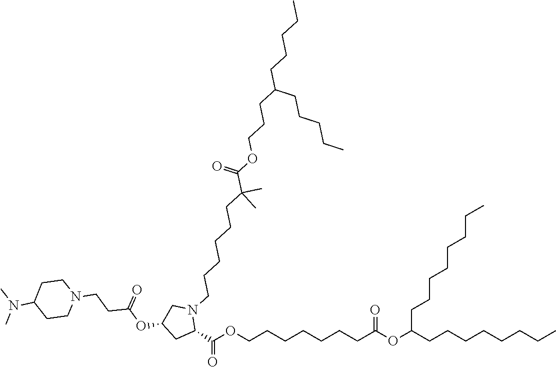 [SMILES: CCCCCCCCC(CCCCCCCC)OC(=O)CCCCCCCOC(=O)[C@@H]1C[C@H](OC(=O)CCN2CCC(N(C)C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3-(4- (dimcthylamino) piperidin-1- yl)propanoyl)oxy) pyrrolidine-2- carboxylate |
| 2507 | 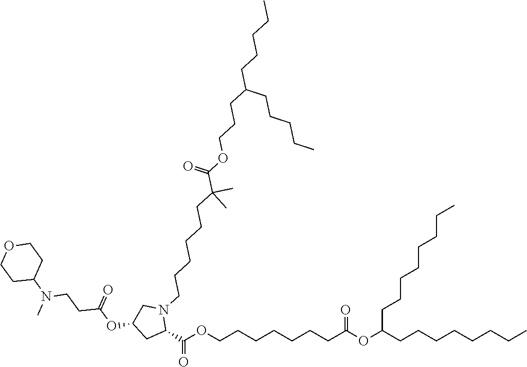 [SMILES: CCCCCCCCC(CCCCCCCC)OC(=O)CCCCCCCOC(=O)[C@@H]1C[C@H](OC(=O)CCN(C)C2CCOCC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3- (methyl(tetrahydro- 2H-pyran-4- yl)amino)propanoyl) oxy)pyrrolidine-2- carboxylate |
| 2508 | 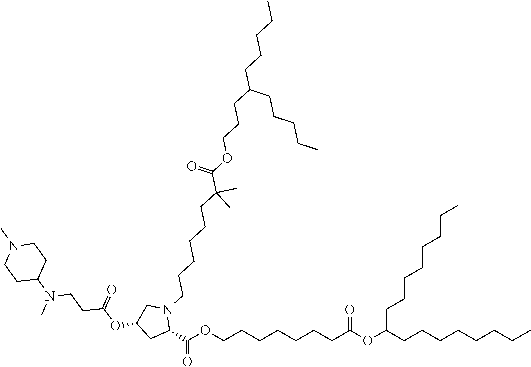 [SMILES: CCCCCCCCC(CCCCCCCC)OC(=O)CCCCCCCOC(=O)[C@@H]1C[C@H](OC(=O)CCN(C)C2CCN(C)CC2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy)octyl)- 4-((3-(methyl(1- methylpiperidin-4- yl)amino)propanoyl) oxy)pyrrolidinc-2- carboxylate |
| 2510 | 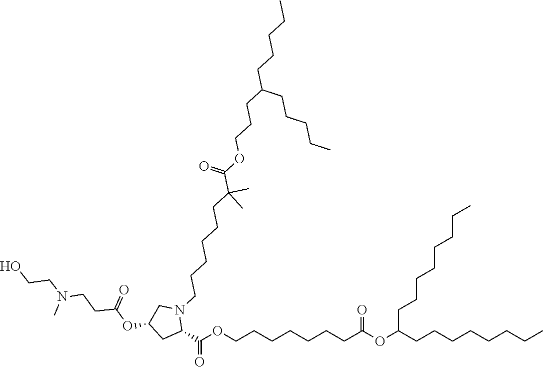 [SMILES: CCCCCCCCC(CCCCCCCC)OC(=O)CCCCCCCOC(=O)[C@@H]1C[C@H](OC(=O)CCN(C)CCO)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4- pentylnonyl)oxy) octyl)-4-((3-((2- hydroxyethyl)(methyl) amino)propanoyl)oxy) pyrrolidine-2- carboxylate |
| 2511 | 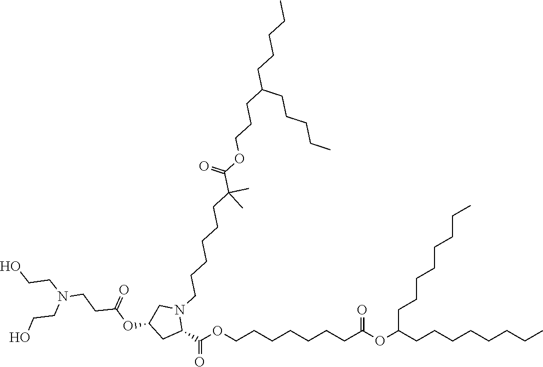 [SMILES: CCCCCCCCC(CCCCCCCC)OC(=O)CCCCCCCOC(=O)[C@@H]1C[C@H](OC(=O)CCN(CCO)CCO)CN1CCCCCCC(C)(C)C(=O)OCCCC(CCCCC)CCCCC] | 8-(heptadecan-9- yloxy)-8-oxooctyl (2S,4S)-4-((3-(bis(2- hydroxyethyl)amino) propanoyl)oxy)-1- (7,7-dimethyl-8-oxo- 8-((4- pentylnonyl)oxy)octyl) pyrrolidine-2- carboxylate |

Additional non-limiting examples of ionizable lipid compounds disclosed here are set forth in Table 2 below.

TABLE 2
Exemplary ionizable lipid compounds.

| Lipid | Structure | IUPAC Name |
|---|---|---|
| 2509 | 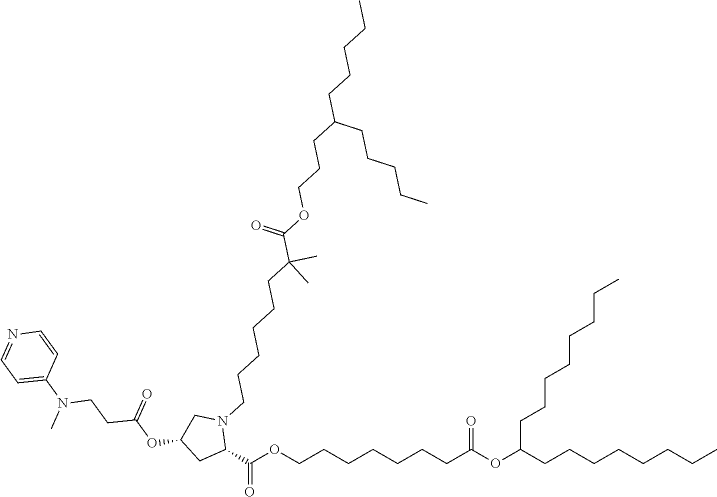 [SMILES: CCCCCCCCC(CCCCCCCC)OC(=O)CCCCCCCOC(=O)[C@@H]1C[C@H](OC(=O)CCN(C)c2ccncc2)CN1CCCCCCC(C)(C)C(=O)OCCCC(CCCCC)CCCCC] | 8-(heptadecan-9- yloxy)-8-oxooctyl (2S,4S)-1-(7,7- dimethyl-8-oxo-8- ((4-pentylnonyl)oxy) octyl)-4-((3- (methyl(pyridin-4- yl)amino)propanoyl) oxy)pyrrolidine-2- carboxylate |
| 2506 | 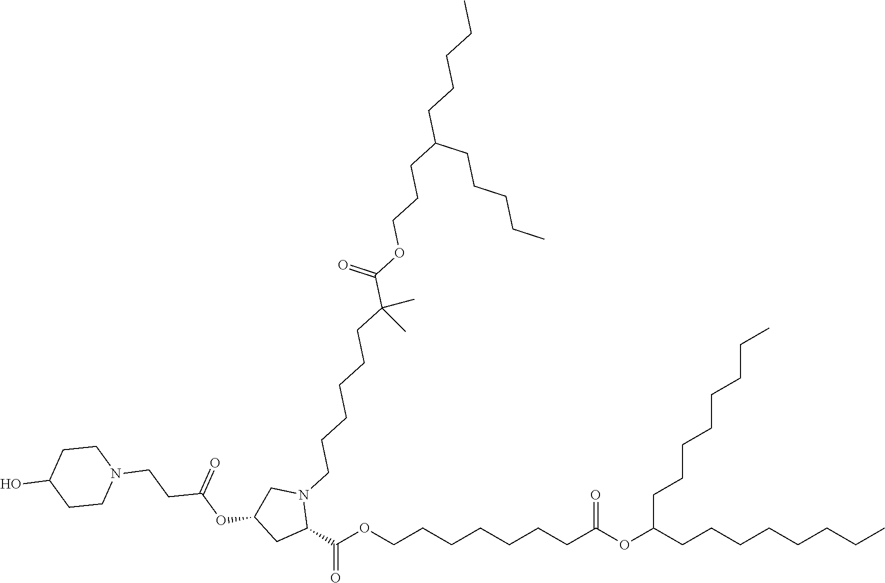 [SMILES: CCCCCCCCC(CCCCCCCC)OC(=O)CCCCCCCOC(=O)[C@@H]1C[C@H](OC(=O)CCN2CCC(O)CC2)CN1CCCCCCC(C)(C)C(=O)OCCCC(CCCCC)CCCCC] | 8-(heptadecan-9- yloxy)-8-oxooctyl (2S,4S)-1-(7,7- dimethy1-8-oxo-8- ((4-pentylnonyl)oxy) octyl)-4-((3-(4- hydroxypiperidin-1- yl)propanoyl)oxy) pyrrolidine- 2-carboxylate |
| 2484 | 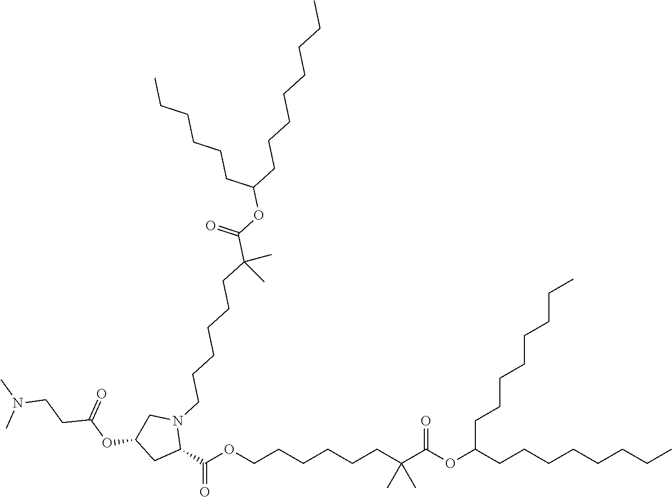 [SMILES: CCCCCCCCC(CCCCCC)OC(=O)C(C)(C)CCCCCCN1C[C@@H](OC(=O)CCN(C)C)C[C@H]1C(=O)OCCCCCCC(C)(C)C(=O)OC(CCCCCCCC)CCCCCCCC] | 8-(heptadecan- 9-yloxy)-7,7- dimethyl-8- oxooctyl (2S,4S)-1- (7,7-dimethy1-8- oxo-8-(tridecan-7- yloxy)octyl)-4-((3- (dimethylamino) propanoyl)oxy) pyrrolidine- 2-carboxylate |
| 2535 | 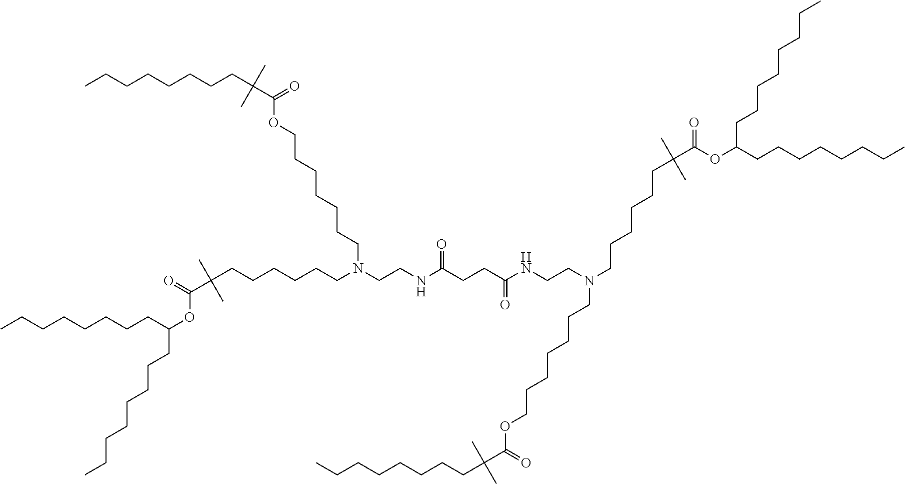 [SMILES: CCCCCCCCC(CCCCCCCC)OC(=O)C(C)(C)CCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] | |
| 2536 | 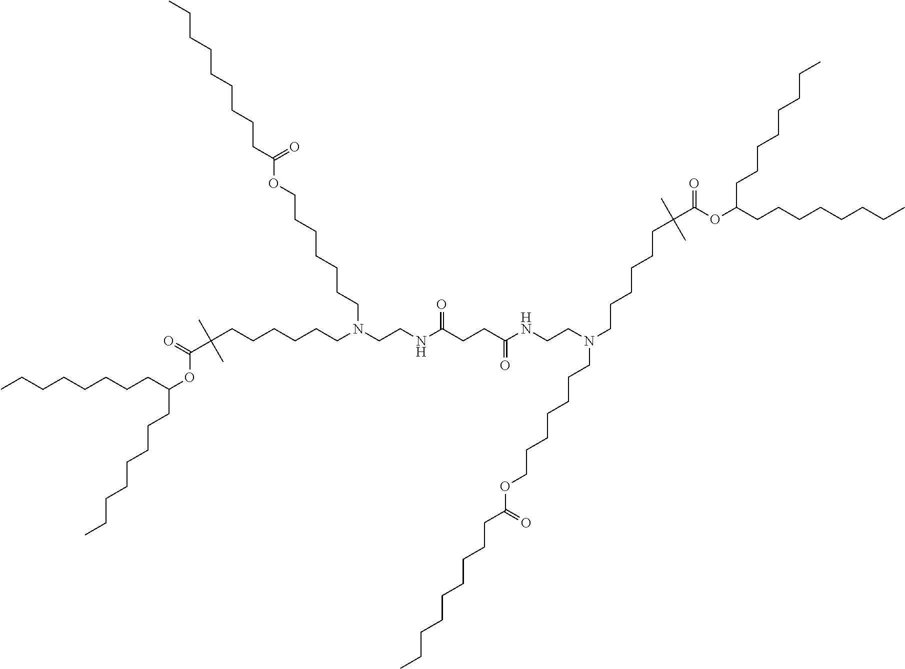 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)CCCCCCCCC)CCCCCCC(C)(C)C(=O)OC(CCCCCCCC)CCCCCCCC] | |
| 2537 | 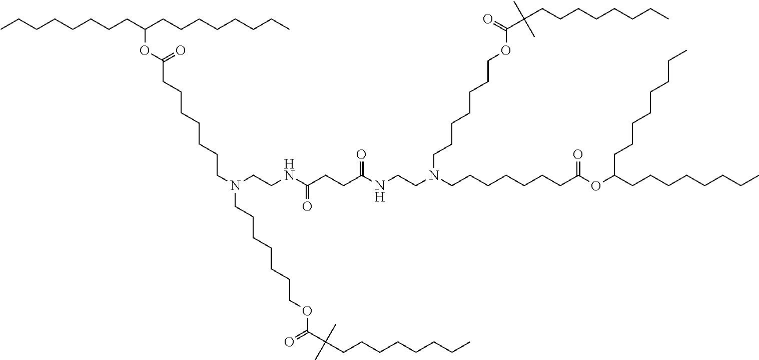 [SMILES: CCCCCCCCC(CCCCCCCC)OC(=O)CCCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] | |
| 2544 | 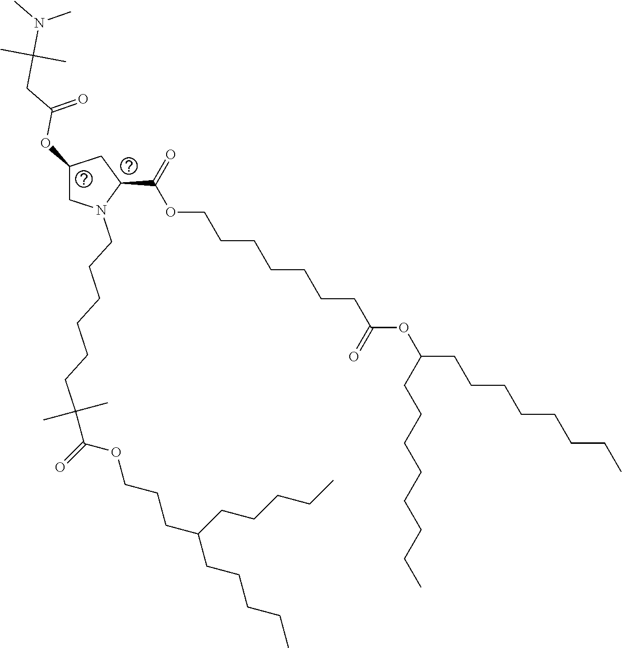 [SMILES: CCCCCCCCC(CCCCCCCC)OC(=O)CCCCCCCOC(=O)[C@@H]1C[C@H](OC(=O)CC(C)(C)N(C)C)CN1CCCCCCC(C)(C)C(=O)OCCCC(CCCCC)CCCCC] | |
| 2545 | 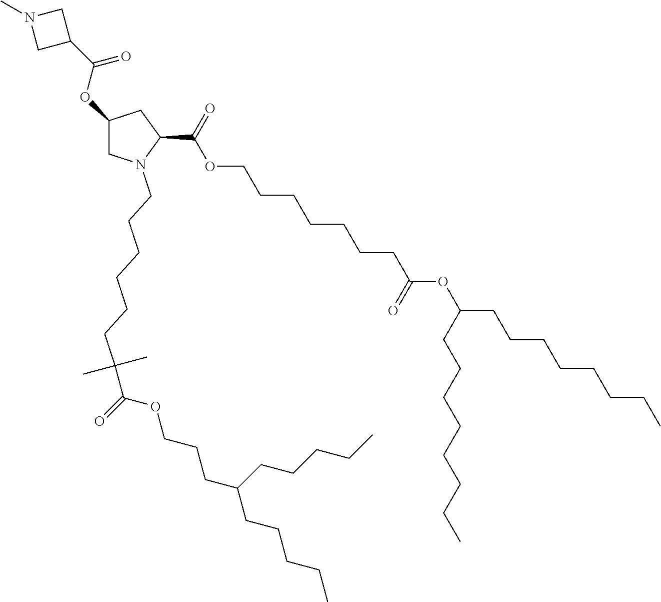 [SMILES: CCCCCCCCC(CCCCCCCC)OC(=O)CCCCCCCOC(=O)[C@@H]1C[C@H](OC(=O)C2CN(C)C2)CN1CCCCCCC(C)(C)C(=O)OCCCC(CCCCC)CCCCC] | |
| 2546 | 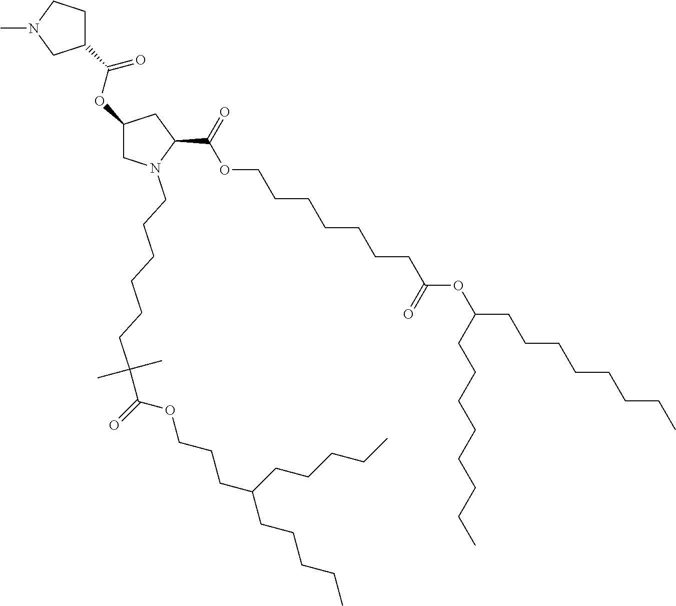 | |
| 2547 | 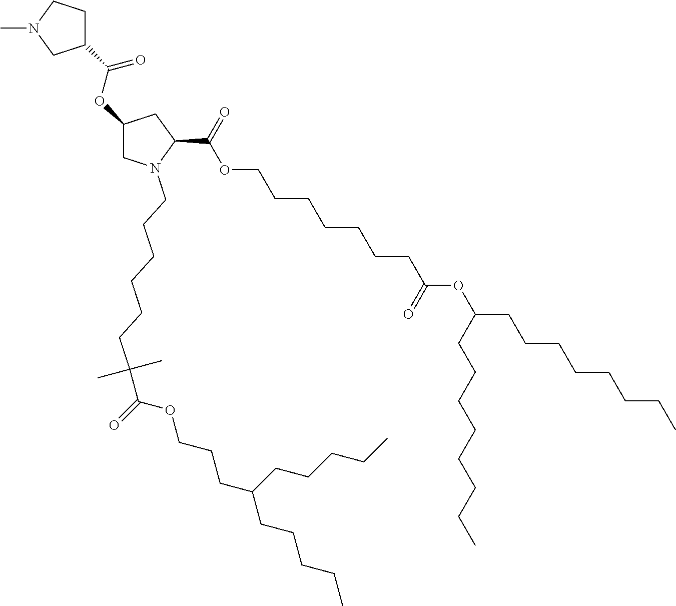 | |
| 2551 | 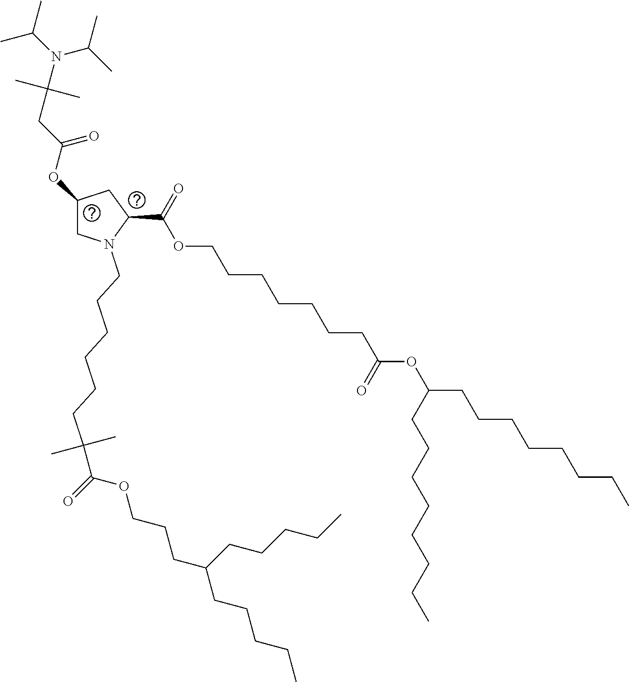 [SMILES: CCCCCCCCC(CCCCCCCC)OC(=O)CCCCCCCOC(=O)[C@@H]1C[C@H](OC(=O)CC(C)(C)N(C(C)C)C(C)C)CN1CCCCCCC(C)(C)C(=O)OCCCC(CCCCC)CCCCC] | |
| 2552 | 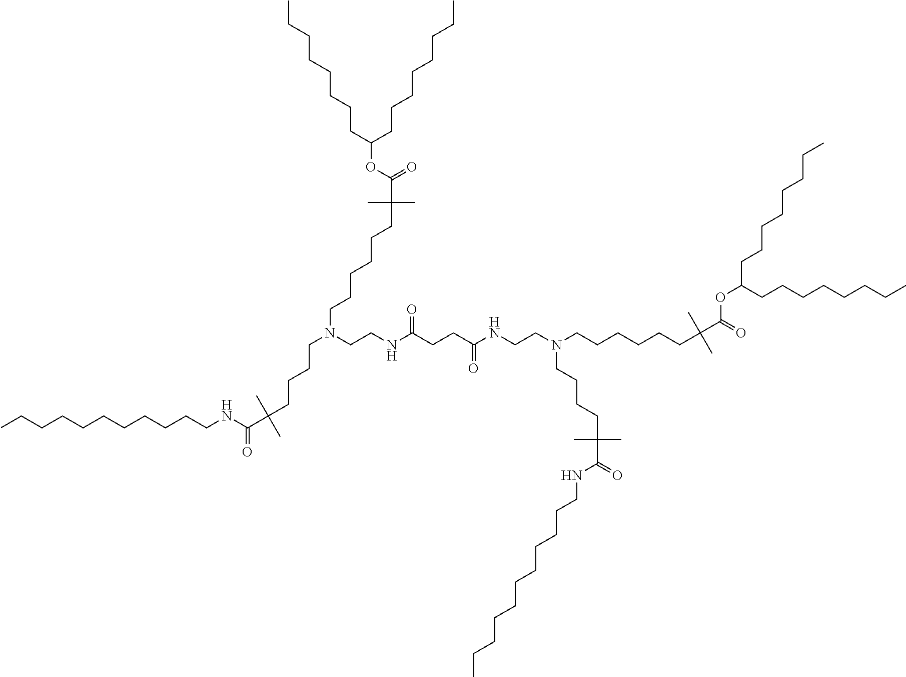 [SMILES: CCCCCCCCCCCNC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)NCCCCCCCCCCC] | |
| 2553 | 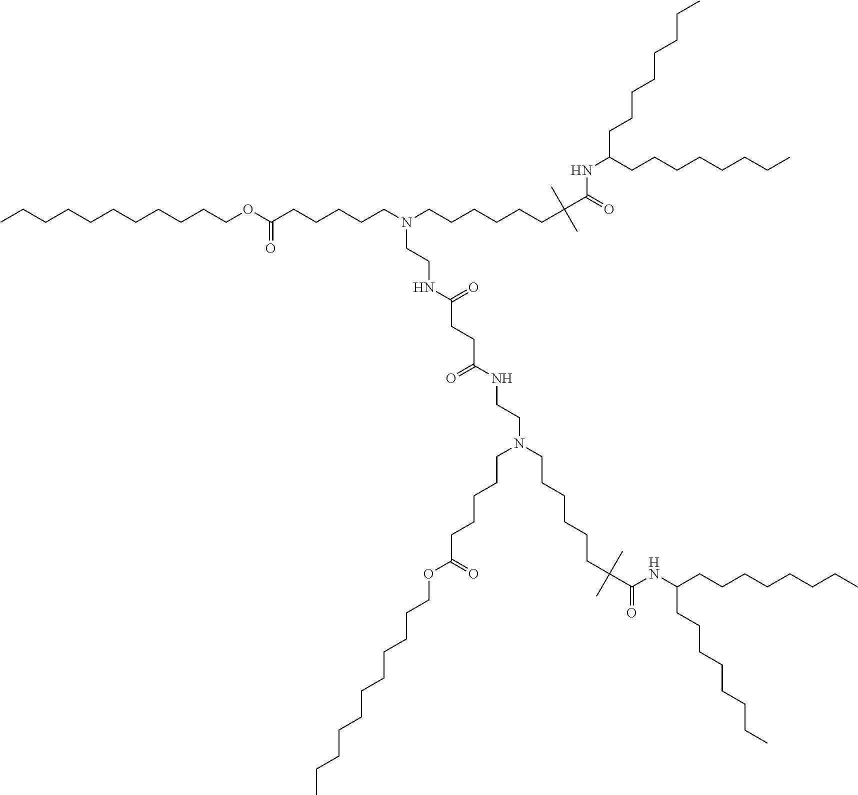 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OCCCCCCCCCCC] | |
| 2554 | 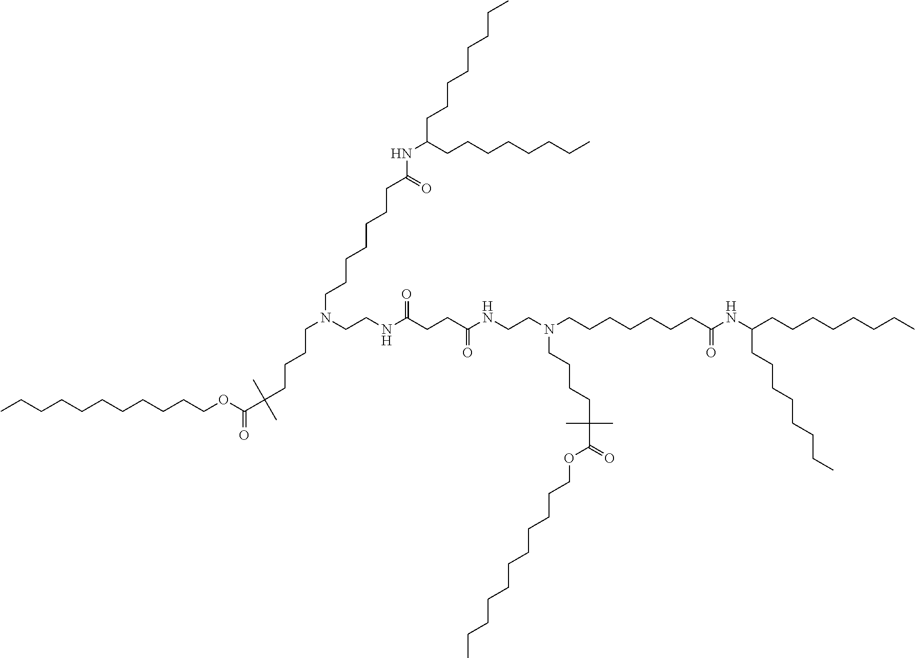 [SMILES: CCCCCCCCCCCOC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | |
| 2555 | 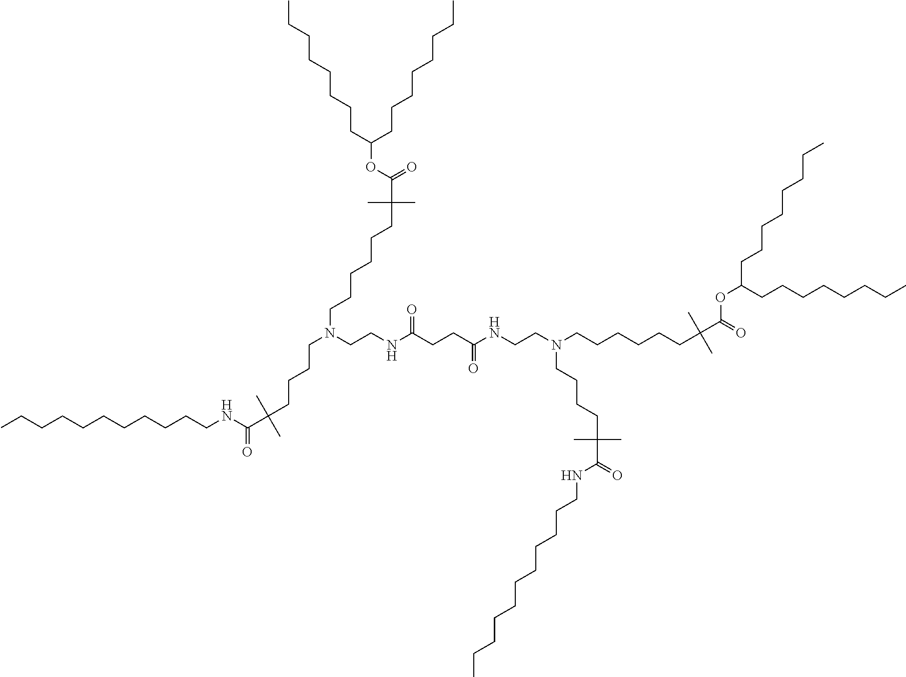 [SMILES: CCCCCCCCCCCNC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)NCCCCCCCCCCC] | |
| 2556 | 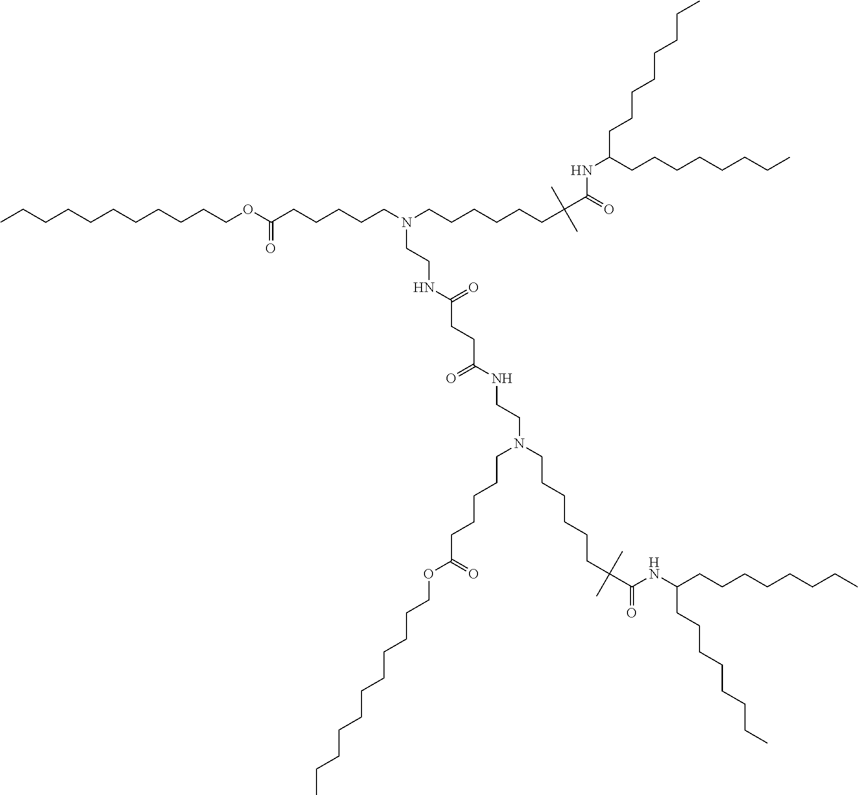 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OCCCCCCCCCCC] | |
| 2557 | 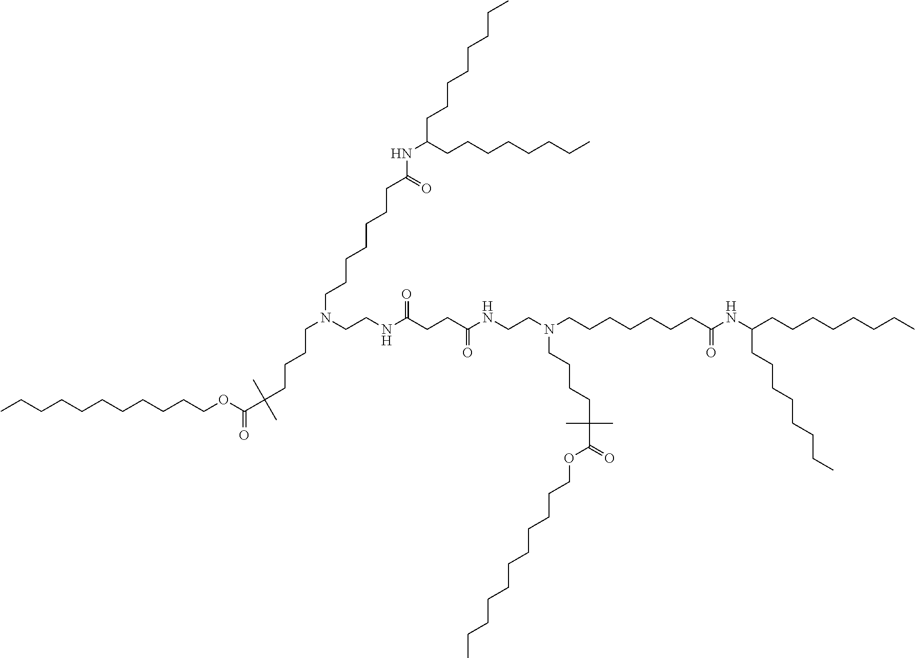 [SMILES: CCCCCCCCCCCOC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | |
| 2559 | 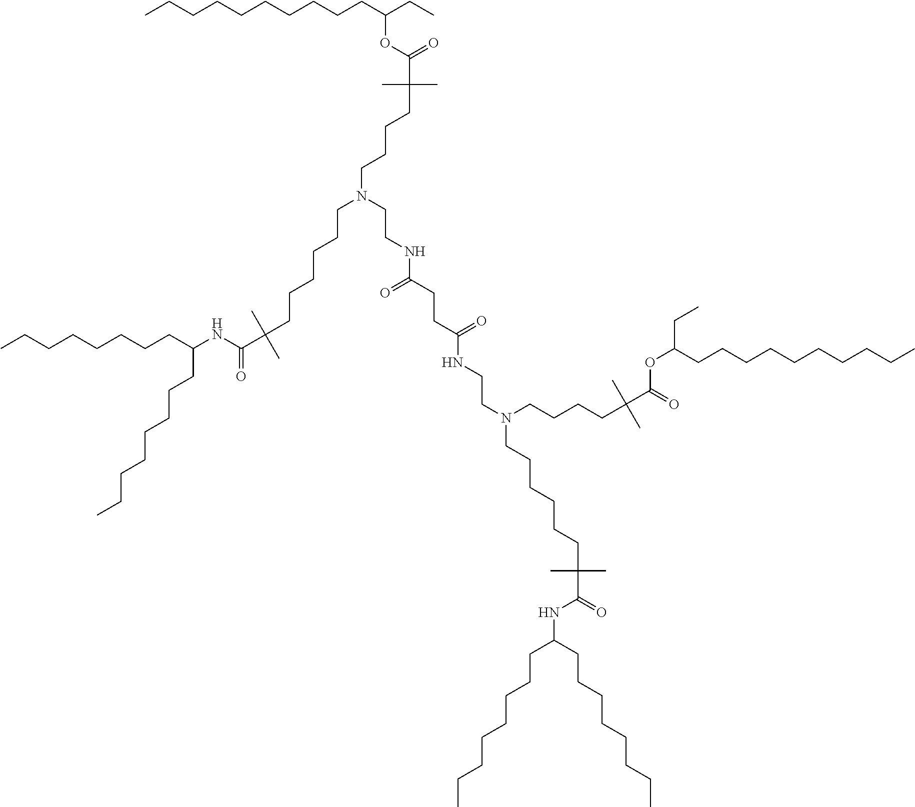 [SMILES: CCCCCCCCCCC(CC)OC(=O)C(C)(C)CCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OC(CC)CCCCCCCCCC] | |
| 2560 | 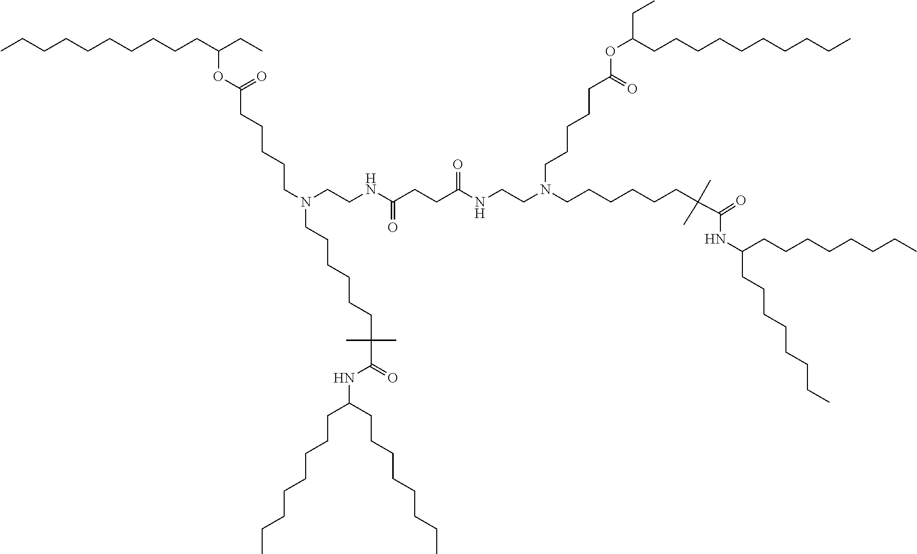 [SMILES: CCCCCCCCCCC(CC)OC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OC(CC)CCCCCCCCCC] | |
| 2561 | 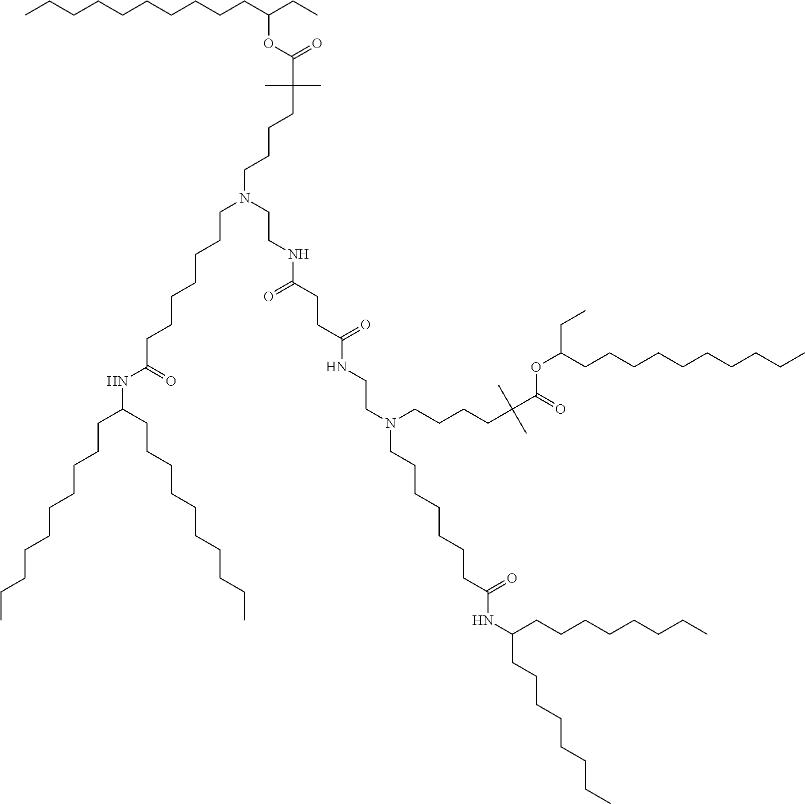 [SMILES: CCCCCCCCCCC(CCCCCCCCCC)NC(=O)CCCCCCCN(CCCCC(C)(C)C(=O)OC(CC)CCCCCCCCCC)CCNC(=O)CCC(=O)NCCN(CCCCCCCC(=O)NC(CCCCCCCC)CCCCCCCC)CCCCC(C)(C)C(=O)OC(CC)CCCCCCCCCC] | |
| 2563 | 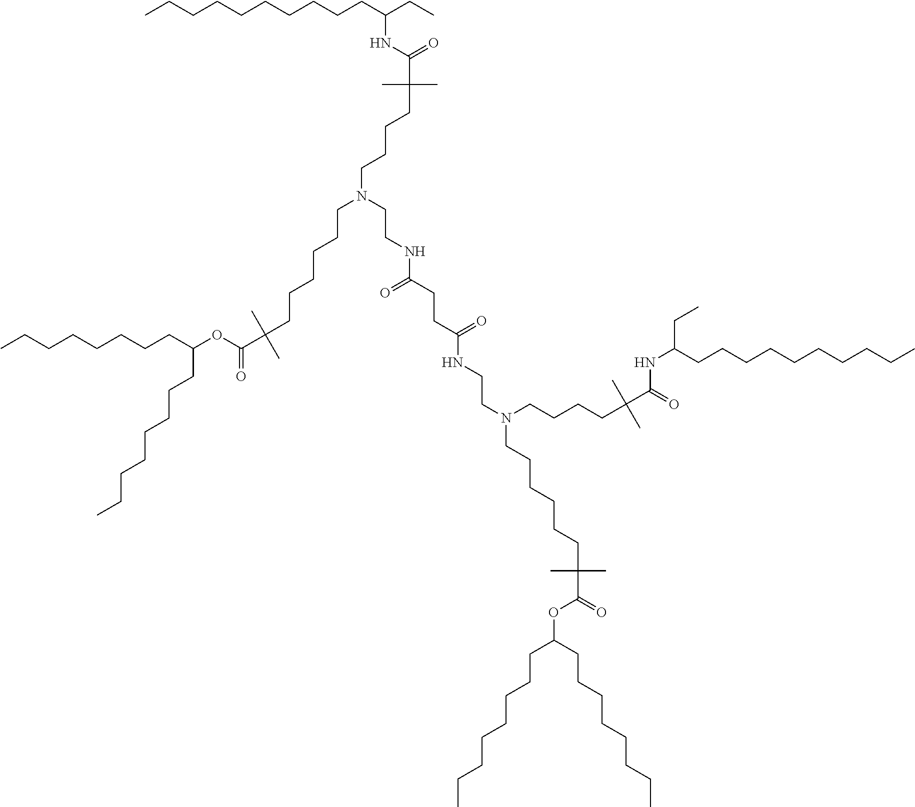 [SMILES: CCCCCCCCCCC(CC)NC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)NC(CC)CCCCCCCCCC] | |
| 2564 | 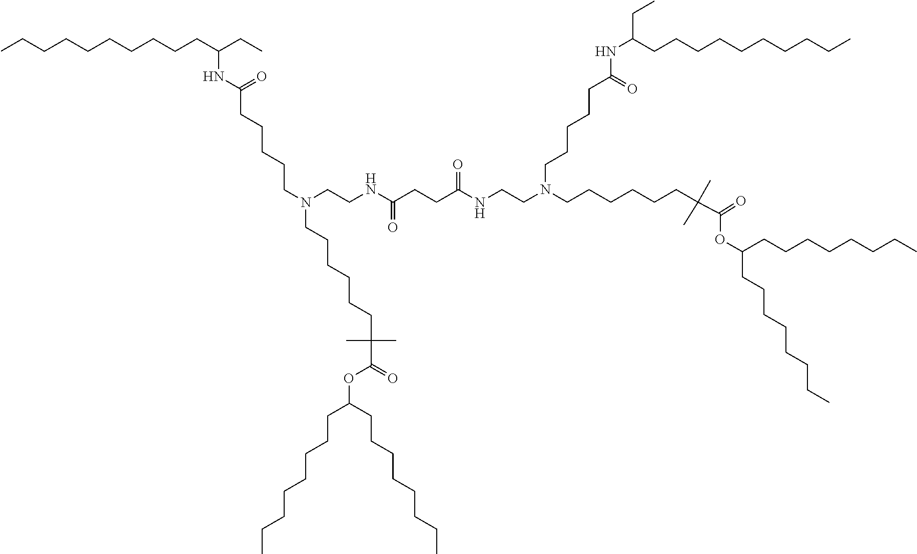 [SMILES: CCCCCCCCCCC(CC)NC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)NC(CC)CCCCCCCCCC] | |
| 2565 | 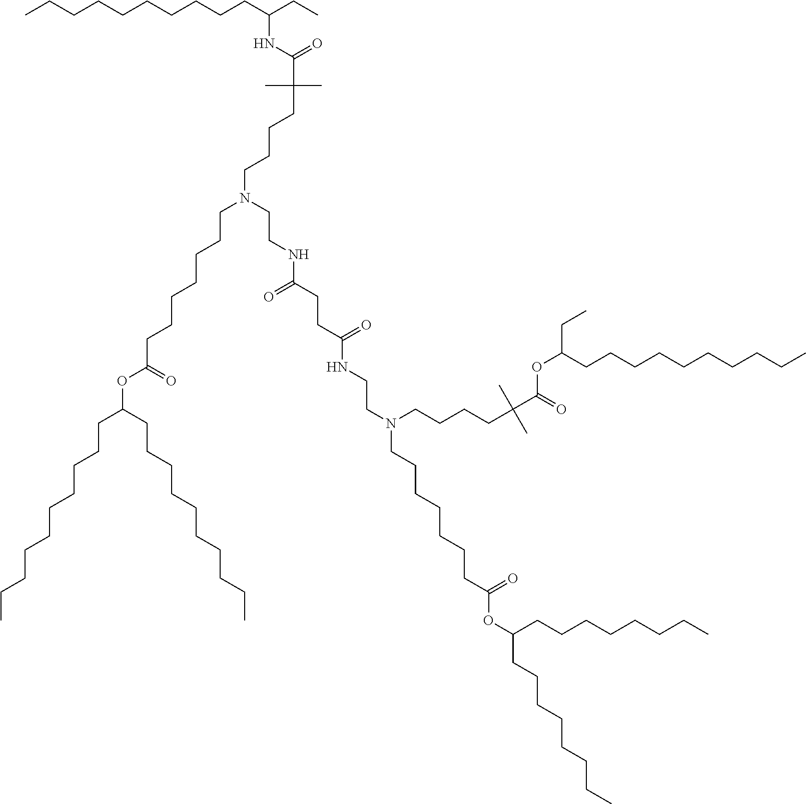 [SMILES: CCCCCCCCCCC(CC)NC(=O)C(C)(C)CCCCN(CCCCCCCC(=O)OC(CCCCCCCCCC)CCCCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] | |
| 2566 | 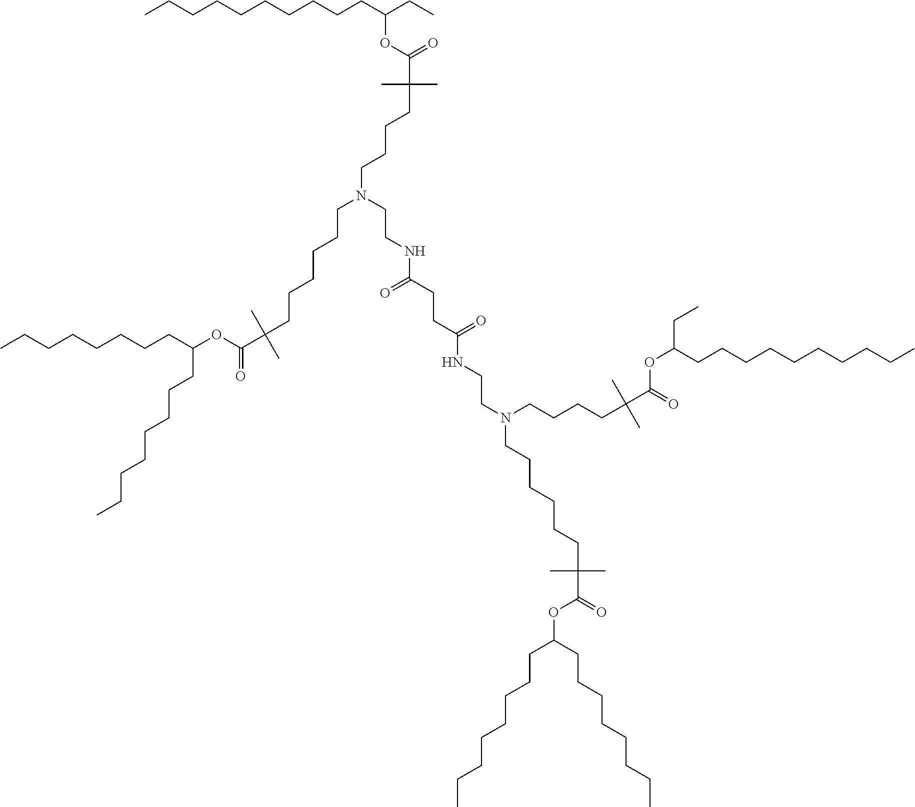 [SMILES: CCCCCCCCCCC(CC)OC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | |
| 2567 | 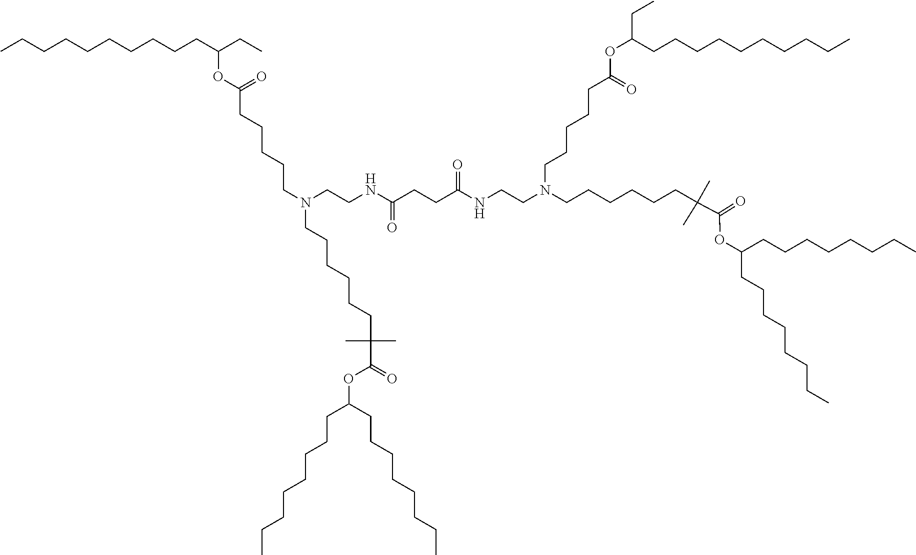 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OC(CC)CCCCCCCCCC] | |
| 2568 | 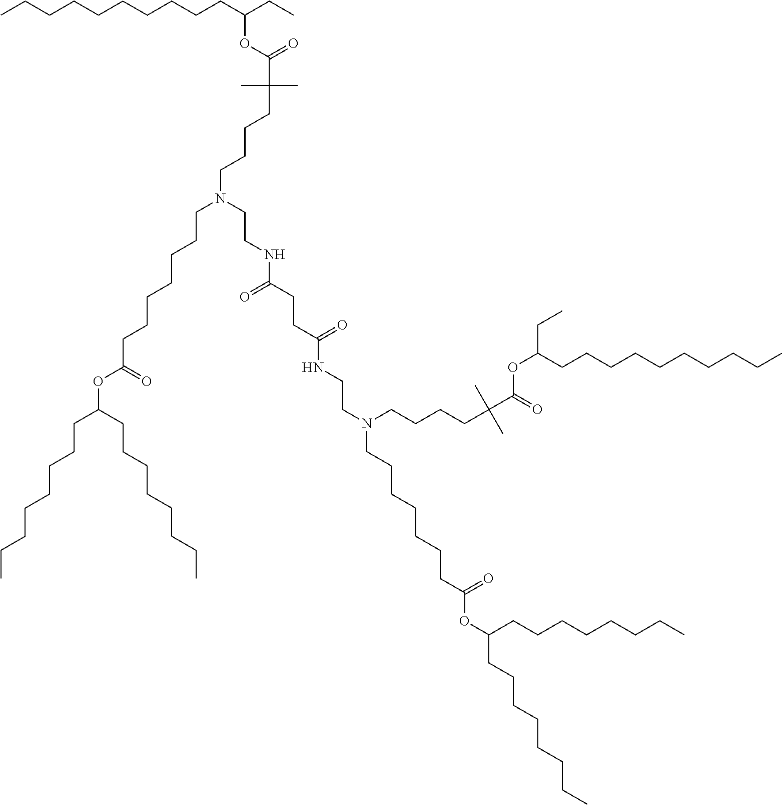 [SMILES: CCCCCCCCCCC(CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] | |
| 2569 | 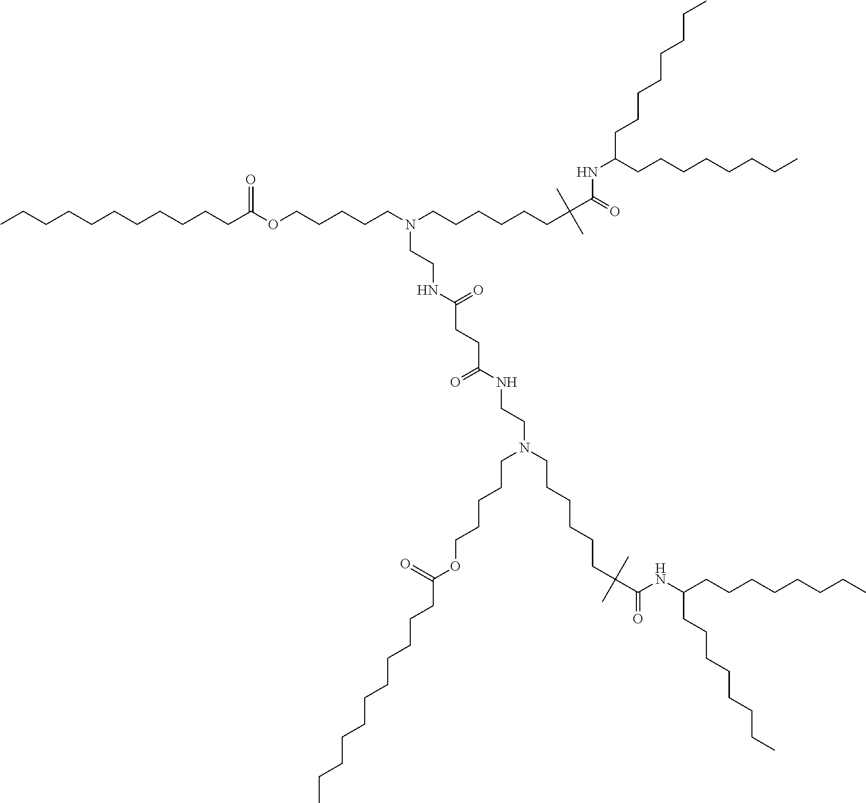 [SMILES: CCCCCCCCCCCC(=O)OCCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCOC(=O)CCCCCCCCCCC] | |
| 2570 | 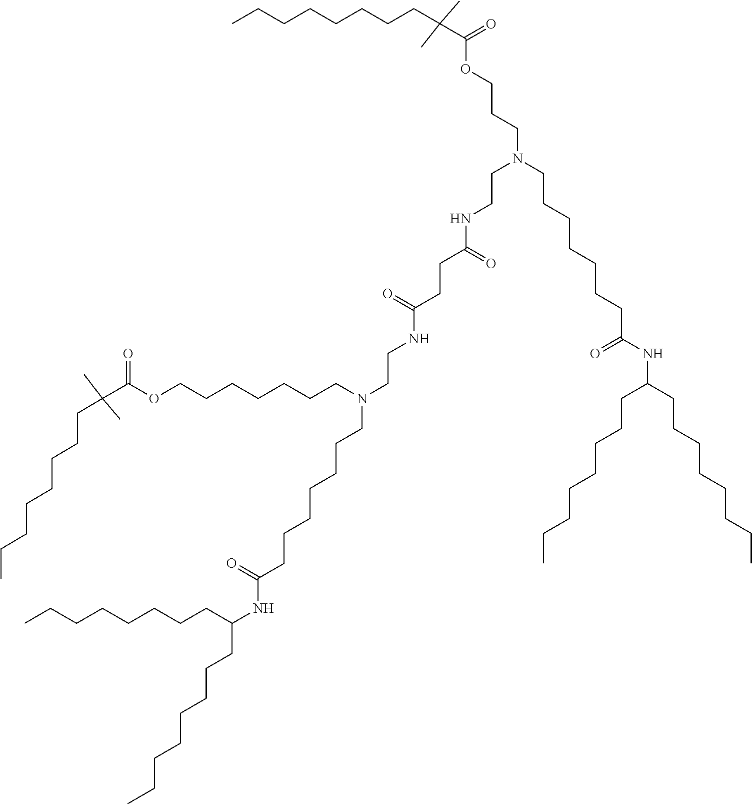 [SMILES: CCCCCCCCC(CCCCCCCC)NC(=O)CCCCCCCN(CCCCCCCOC(=O)C(C)(C)CCCCCCCC)CCNC(=O)CCC(=O)NCCN(CCCCCCCC(=O)NC(CCCCCCCC)CCCCCCCC)CCCOC(=O)C(C)(C)CCCCCCCC] | |
| 2571 | 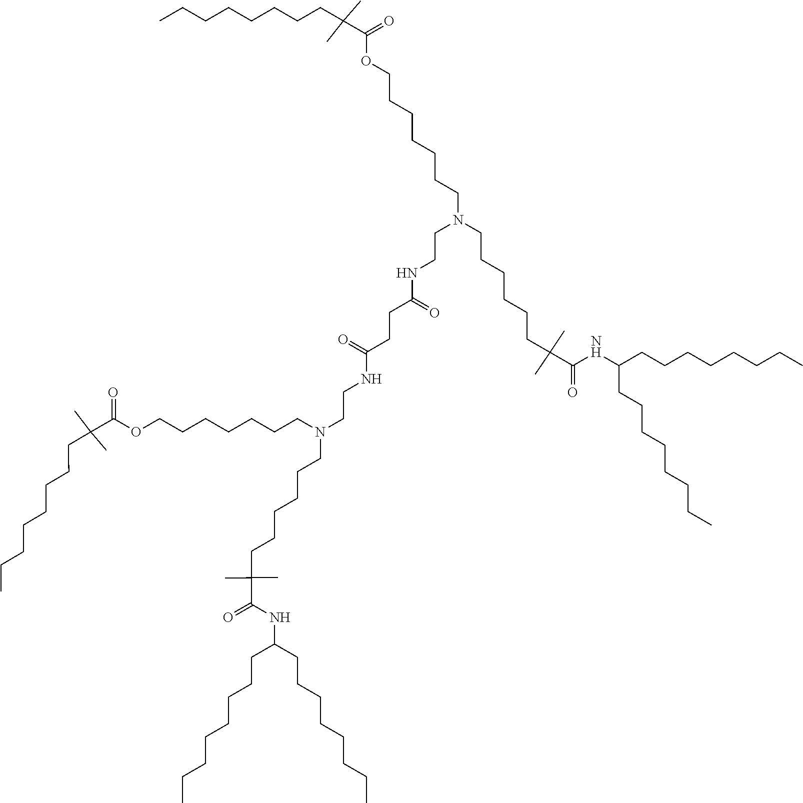 [SMILES: CCCCCCCCC(CCCCCCCC)NC(=O)C(C)(C)CCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)NC(CCCCCCCC)CCCCCCCC] | |
| 2573 | 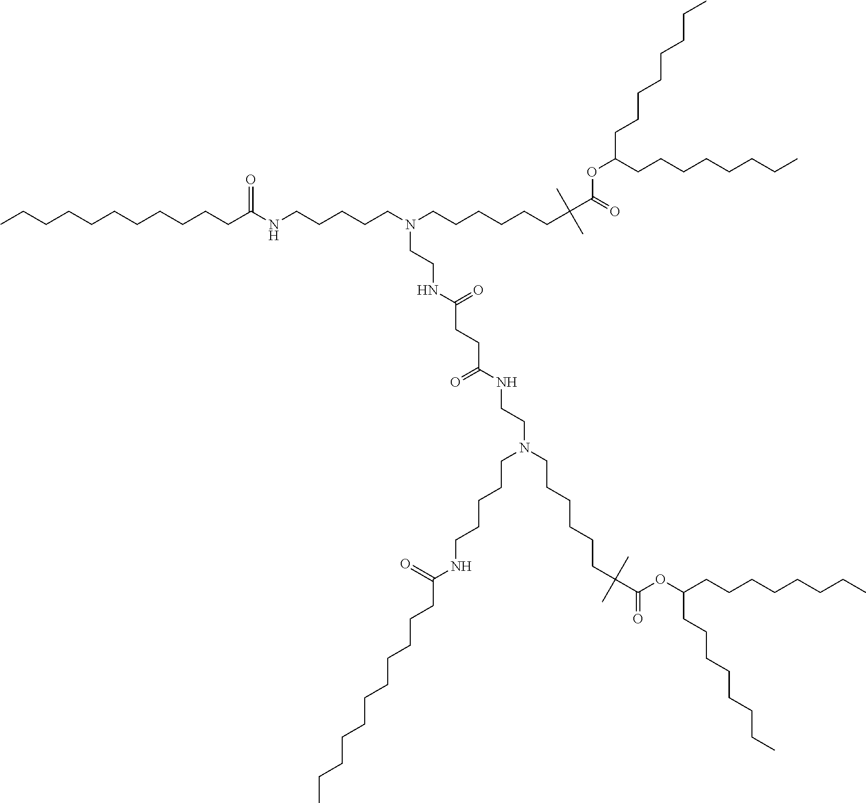 [SMILES: CCCCCCCCCCCC(=O)NCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCNC(=O)CCCCCCCCCCC] | |
| 2574 | 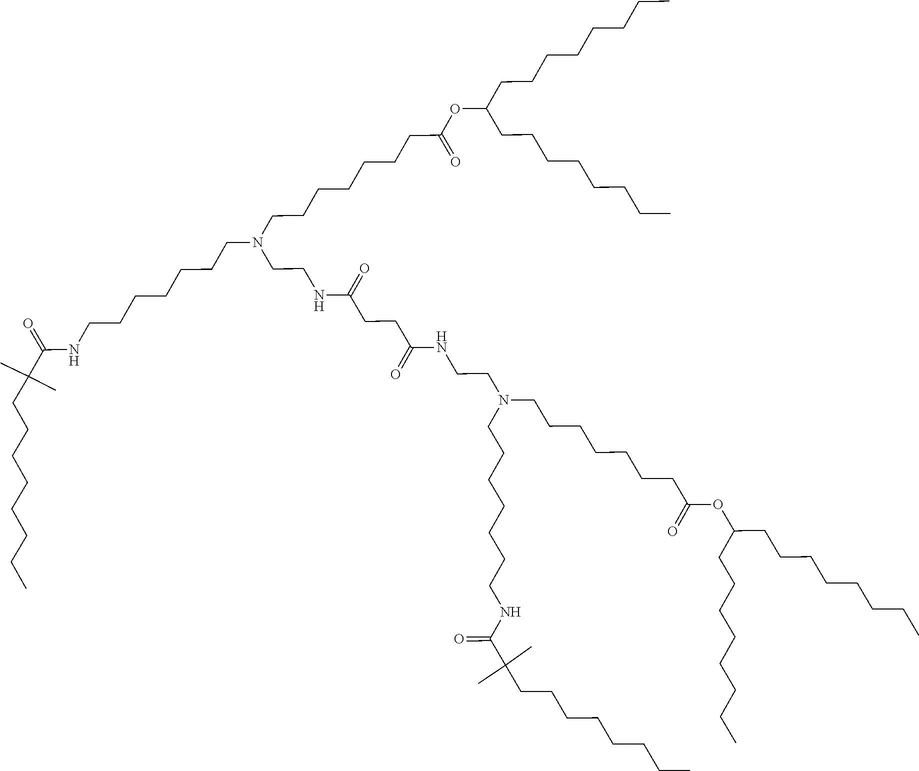 [SMILES: CCCCCCCCC(CCCCCCCC)OC(=O)CCCCCCCN(CCCCCCCNC(=O)C(C)(C)CCCCCCCC)CCNC(=O)CCC(=O)NCCN(CCCCCCCNC(=O)C(C)(C)CCCCCCCC)CCCCCCCC(=O)OC(CCCCCCCC)CCCCCCCC] | |
| 2575 | 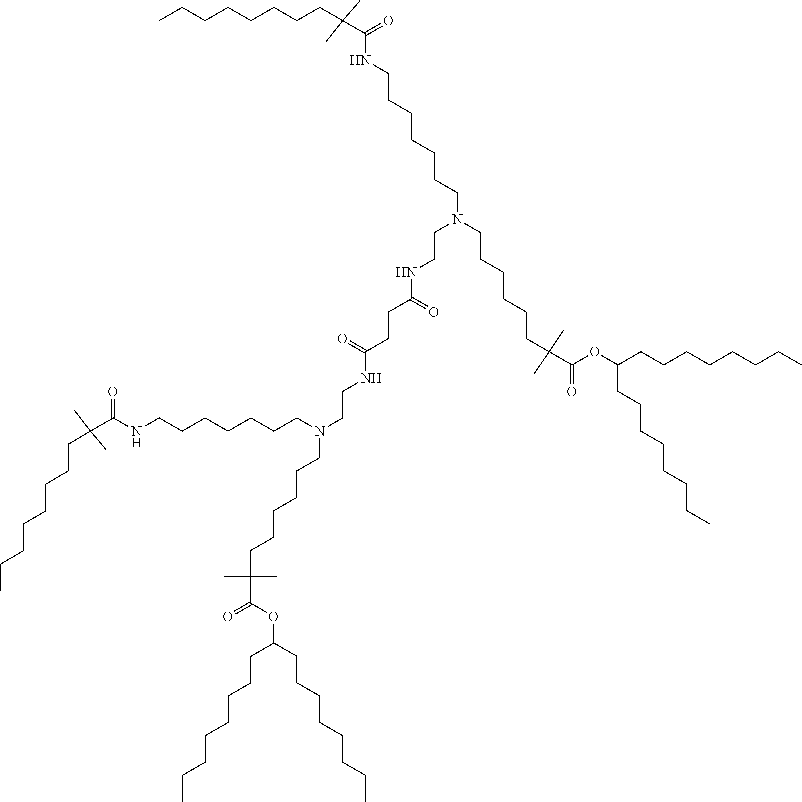 [SMILES: CCCCCCCCC(CCCCCCCC)OC(=O)C(C)(C)CCCCCCN(CCCCCCCNC(=O)C(C)(C)CCCCCCCC)CCNC(=O)CCC(=O)NCCN(CCCCCCCNC(=O)C(C)(C)CCCCCCCC)CCCCCCC(C)(C)C(=O)OC(CCCCCCCC)CCCCCCCC] | |
| 2577 | 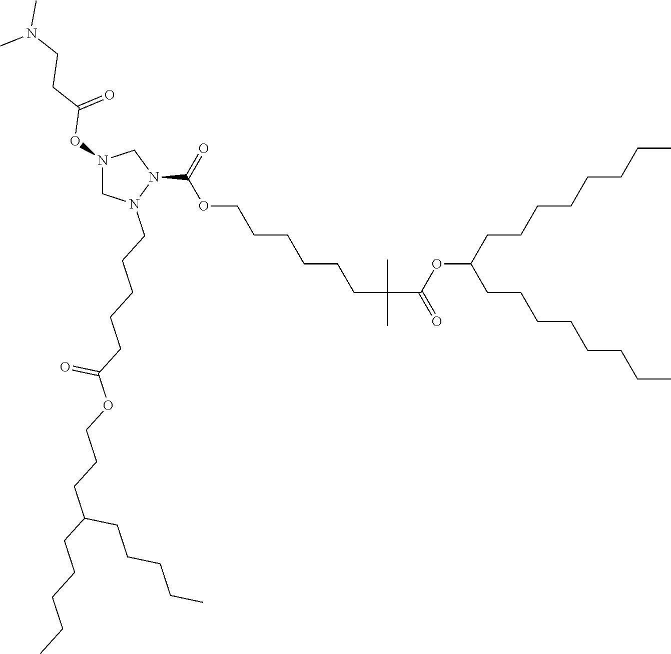 | |
| 2578 | 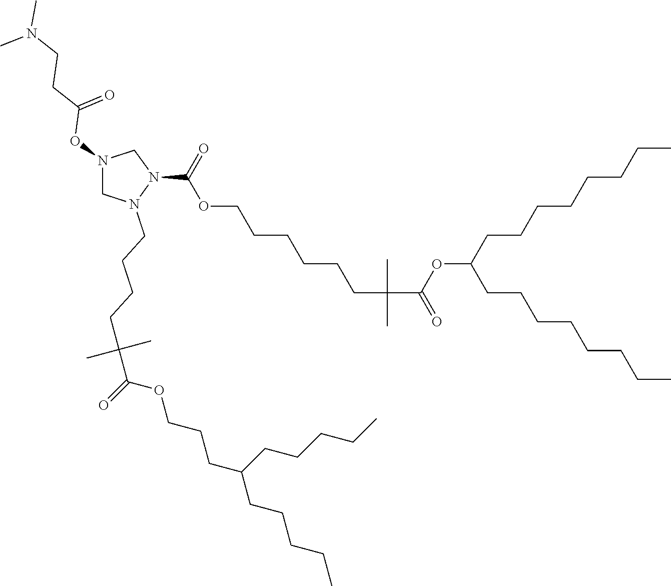 | |
| 2580 | 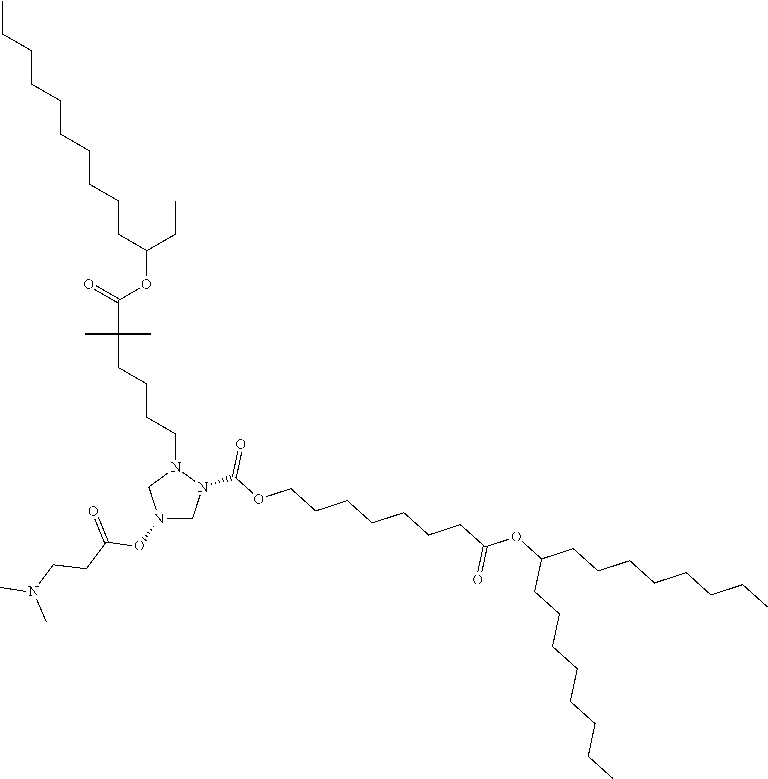 | |
| 2581 | 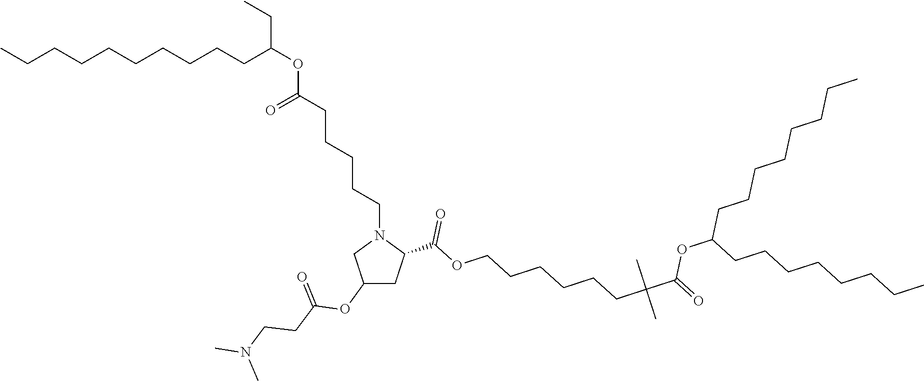 [SMILES: CCCCCCCCCCC(CC)OC(=O)CCCCCN1CC(OC(=O)CCN(C)C)C[C@H]1C(=O)OCCCCCCC(C)(C)C(=O)OC(CCCCCCCC)CCCCCCCC] | |
| 2582 | 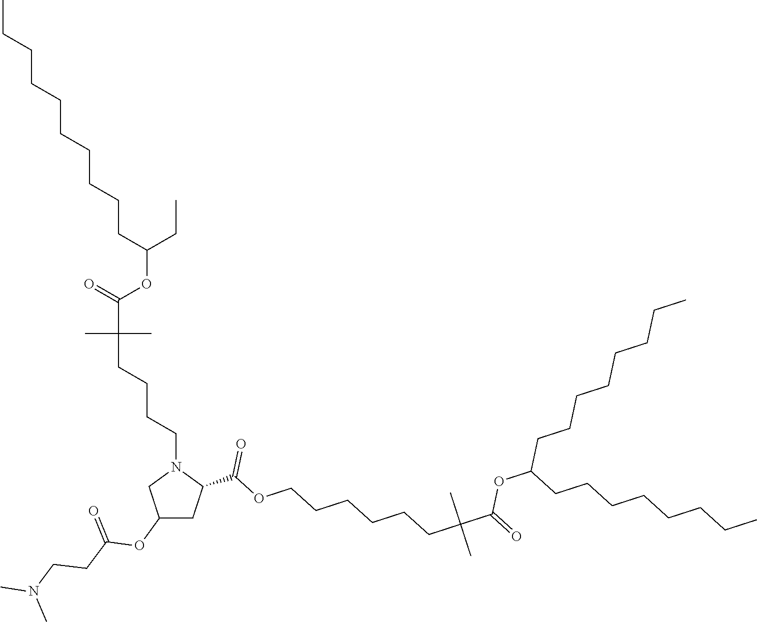 [SMILES: CCCCCCCCCCC(CC)OC(=O)C(C)(C)CCCCN1CC(OC(=O)CCN(C)C)C[C@H]1C(=O)OCCCCCCC(C)(C)C(=O)OC(CCCCCCCC)CCCCCCCC] | |
| 2583 | 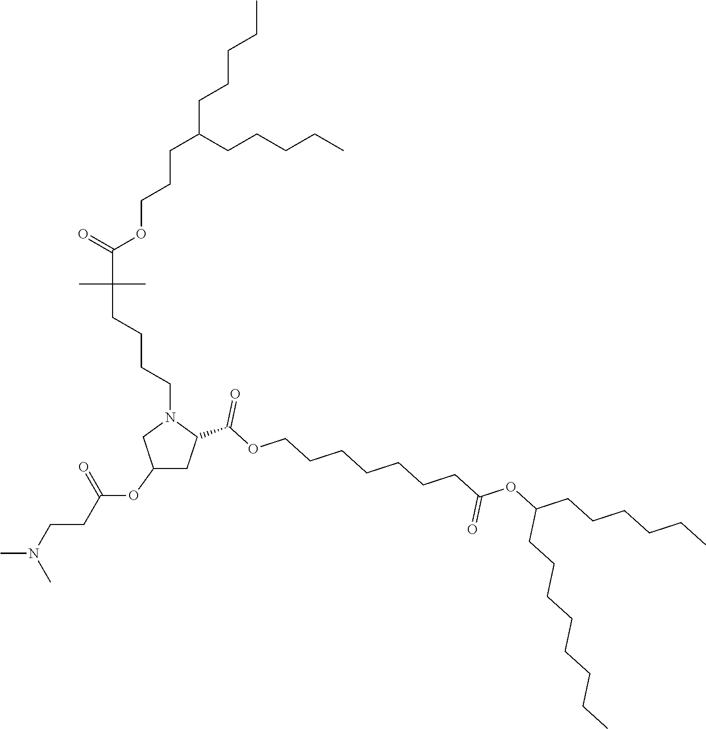 | |
| 2584 | 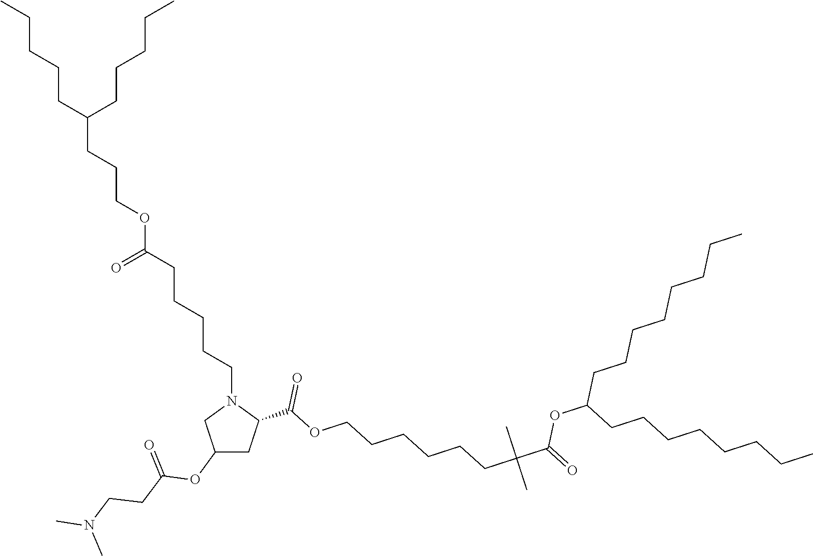 [SMILES: CCCCCCCCC(CCCCCCCC)OC(=O)C(C)(C)CCCCCCOC(=O)[C@@H]1CC(OC(=O)CCN(C)C)CN1CCCCCC(=O)OCCCC(CCCCC)CCCCC] | |
| 2585 | 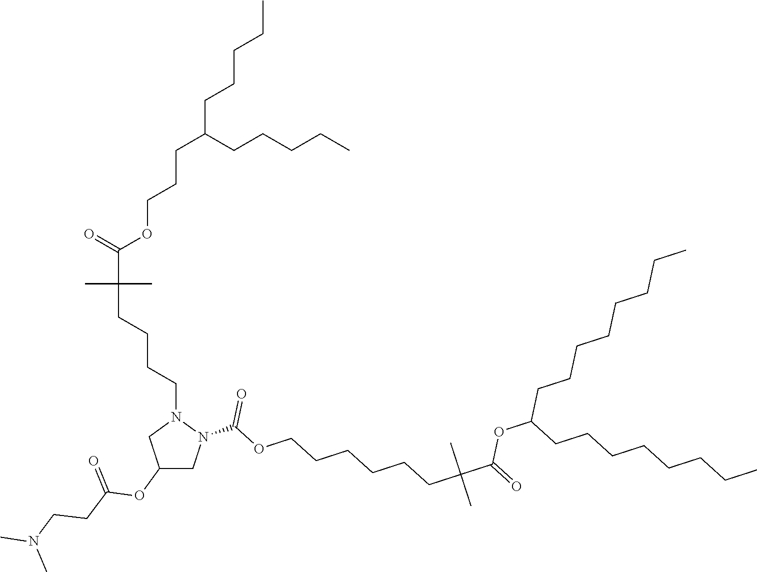 | |
| 2586 | 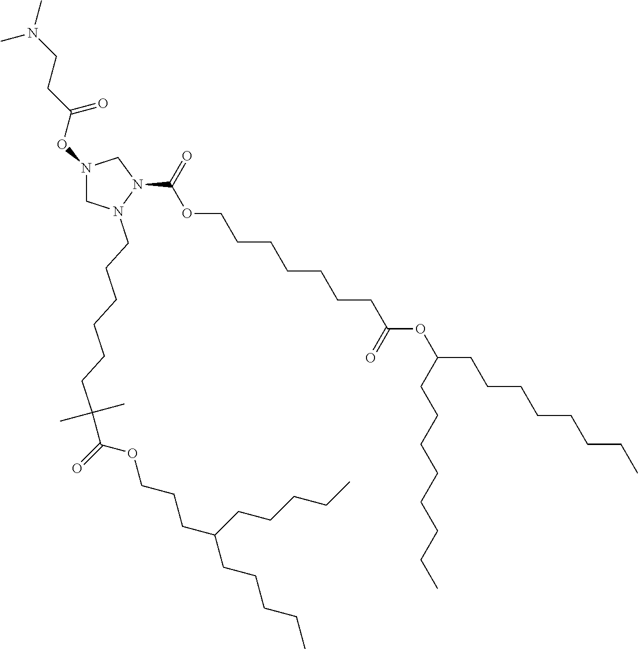 | |
| 2587 | 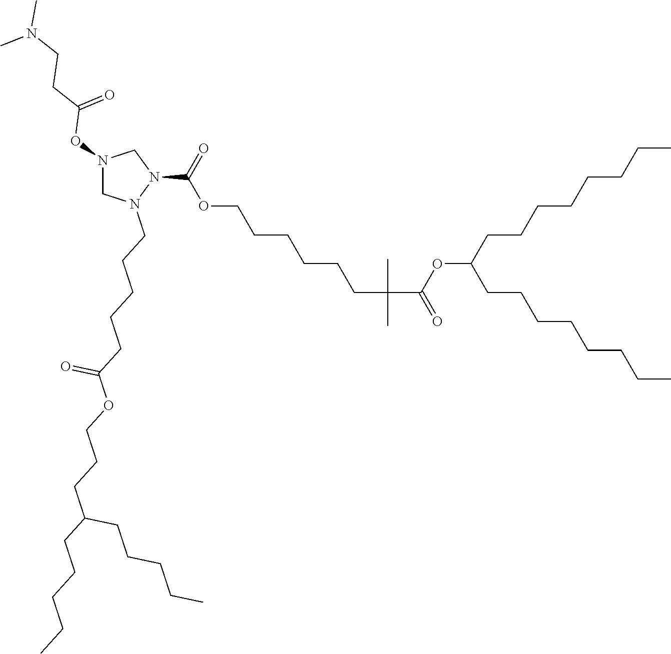 | |
| 2588 | 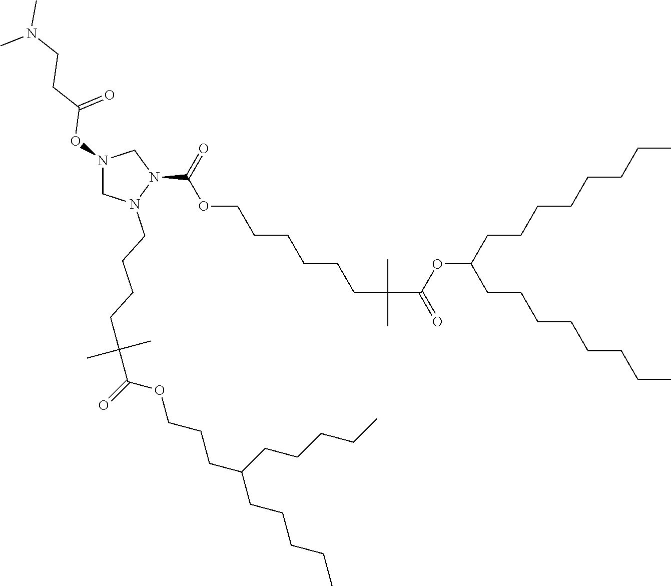 | |
| 2589 | 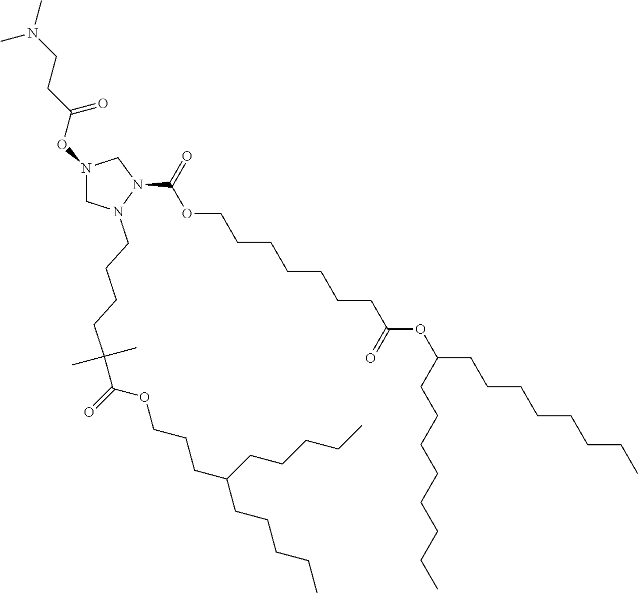 | |
| 2590 | 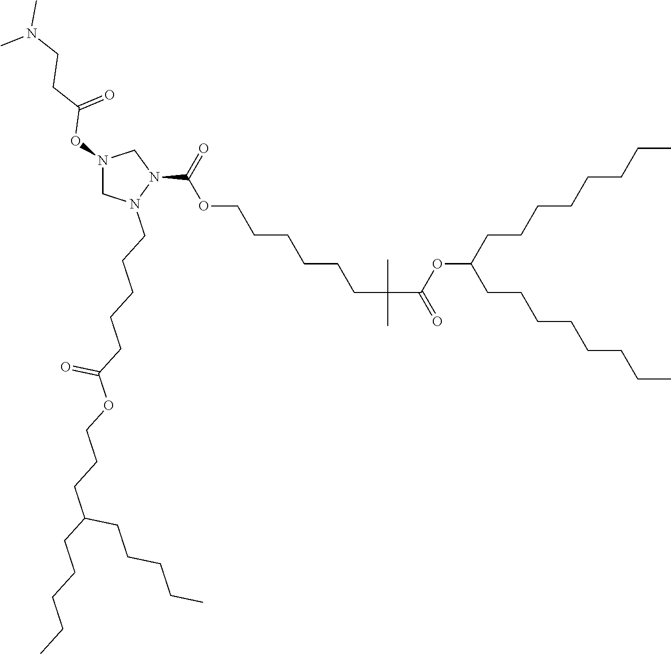 | |
| 2591 | 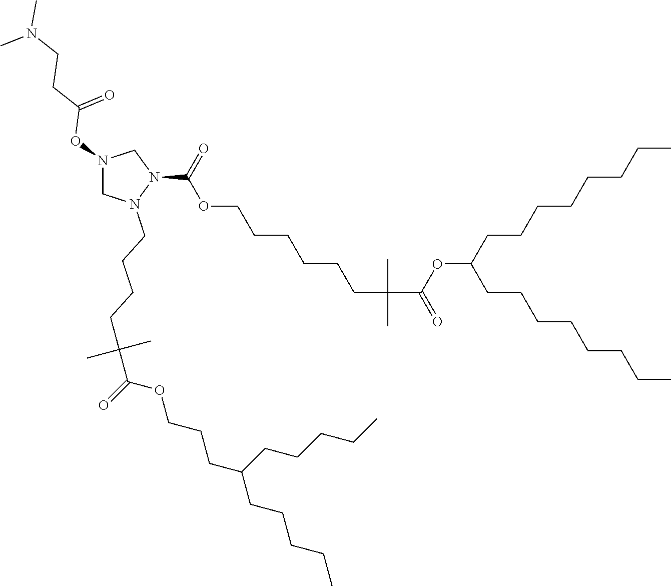 | |
| 2592 | 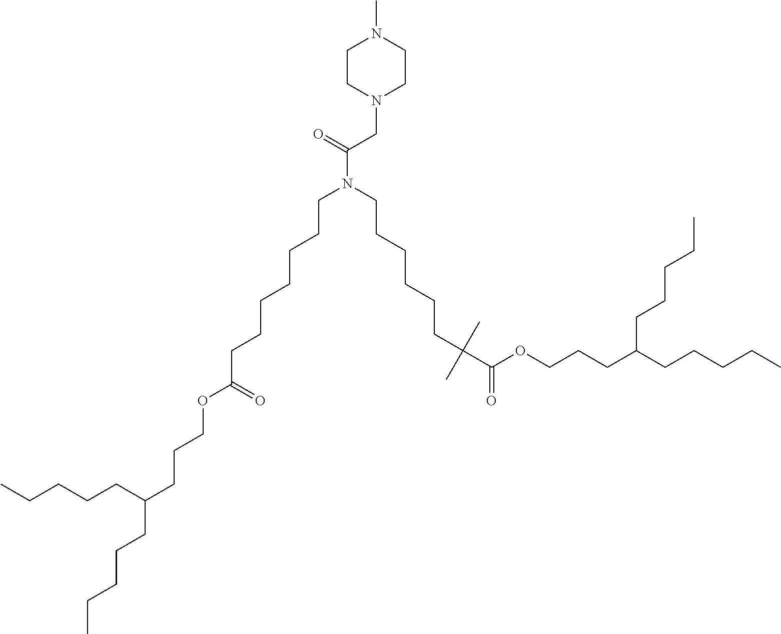 [SMILES: CCCCCC(CCCCC)CCCOC(=O)CCCCCCCN(CCCCCCC(C)(C)C(=O)OCCCC(CCCCC)CCCCC)C(=O)CN1CCN(C)CC1] | |
| 2599 | 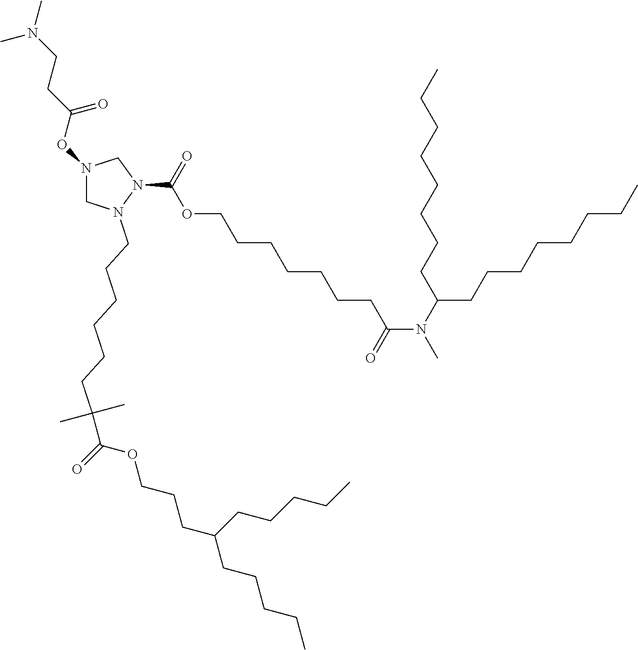 | |
| 2600 | 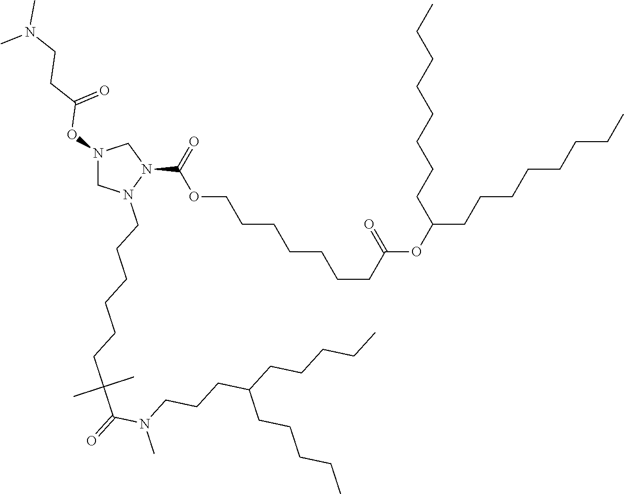 | |
| 2601 | 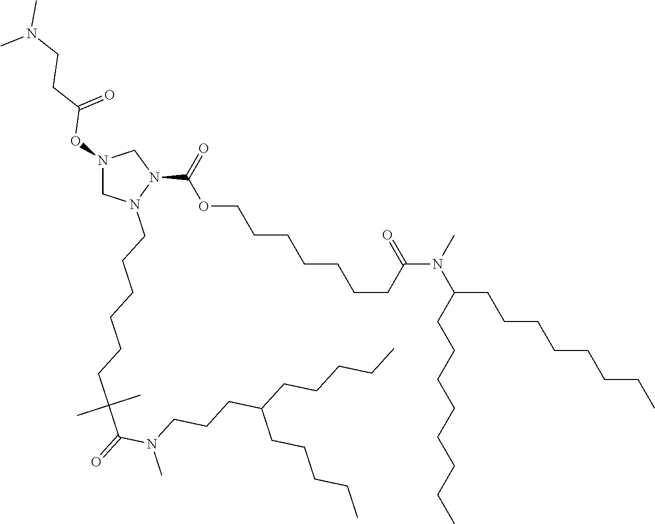 | |
| 2608 | 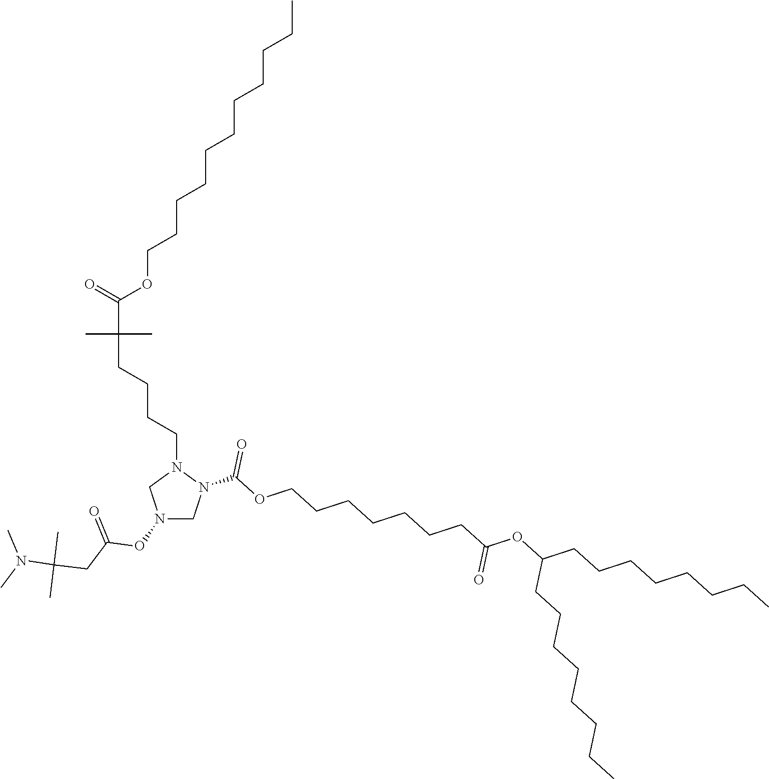 | |
| 2609 | 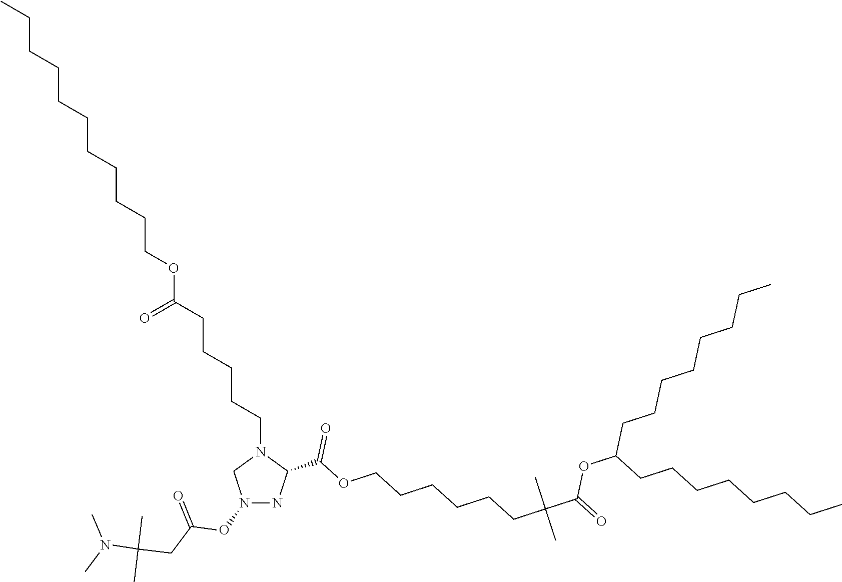 | |
| 2610 | 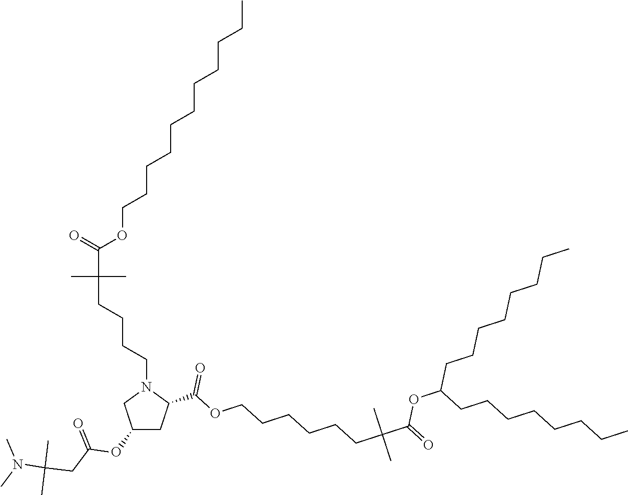 [SMILES: CCCCCCCCCCCOC(=O)C(C)(C)CCCCN1C[C@@H](OC(=O)CC(C)(C)N(C)C)C[C@H]1C(=O)OCCCCCCC(C)(C)C(=O)OC(CCCCCCCC)CCCCCCCC] | |
| 2611 | 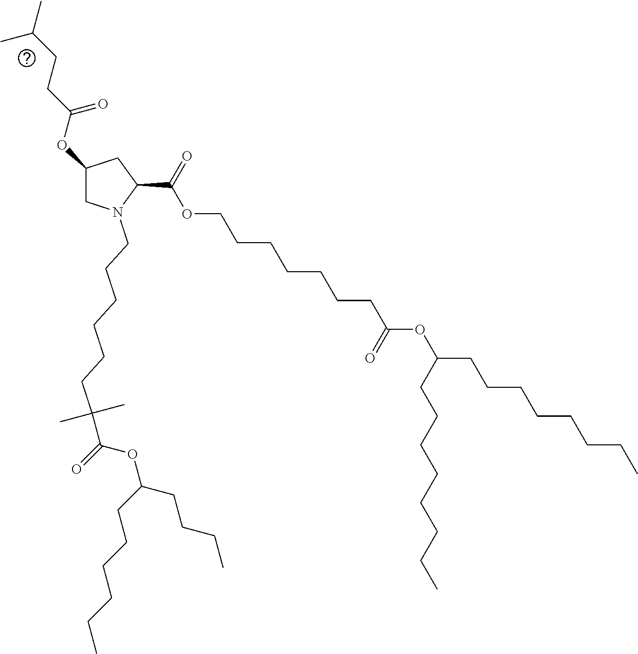 [SMILES: CCCCCCCCC(CCCCCCCC)OC(=O)CCCCCCCOC(=O)[C@@H]1C[C@H](OC(=O)CCC(C)C)CN1CCCCCCC(C)(C)C(=O)OC(CCCC)CCCCCC] | |
| 2612 | 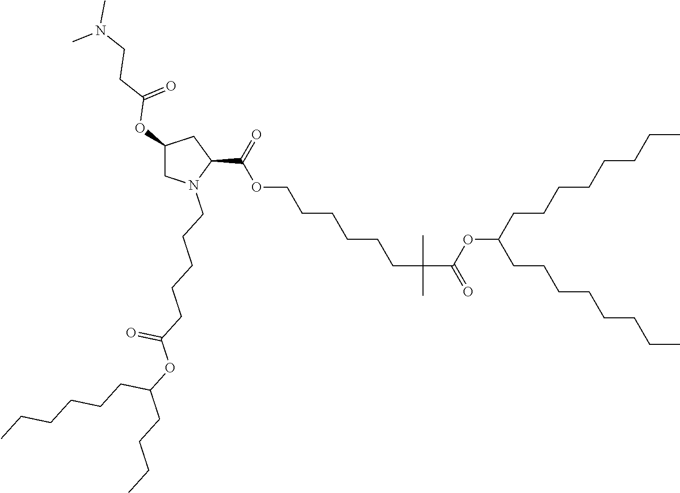 [SMILES: CCCCCCCCC(CCCCCCCC)OC(=O)C(C)(C)CCCCCCOC(=O)[C@@H]1C[C@H](OC(=O)CCN(C)C)CN1CCCCCC(=O)OC(CCCC)CCCCCC] | |
| 2613 | 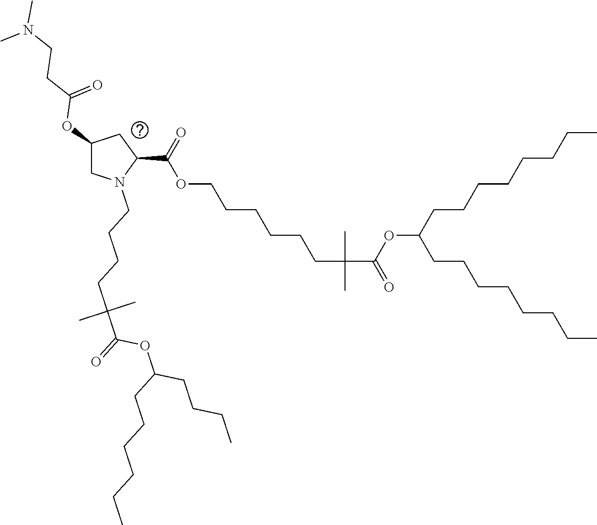 [SMILES: CCCCCCCCC(CCCCCCCC)OC(=O)C(C)(C)CCCCCCOC(=O)[C@@H]1C[C@H](OC(=O)CCN(C)C)CN1CCCCC(C)(C)C(=O)OC(CCCC)CCCCCC] | |
| A1 | 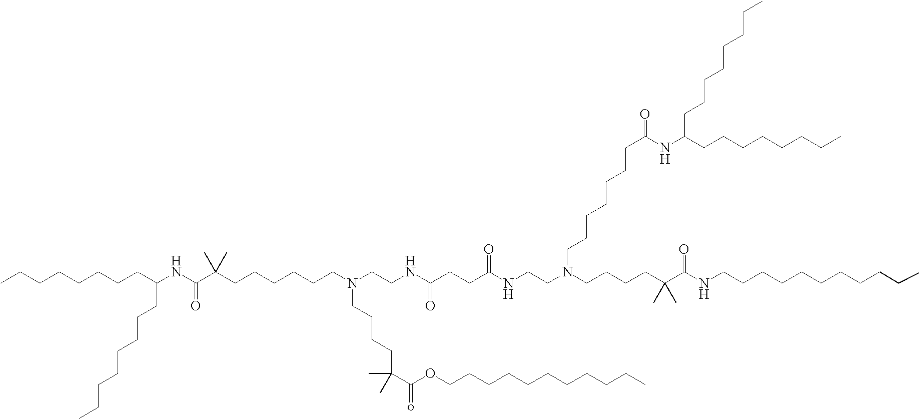 [SMILES: CCCCCCCCCCCNC(=O)C(C)(C)CCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | |
| A2 | 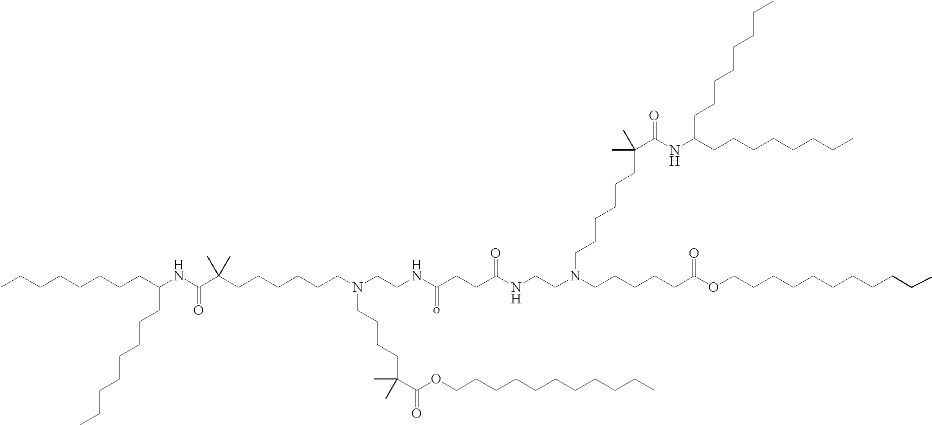 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | |
| A3 | 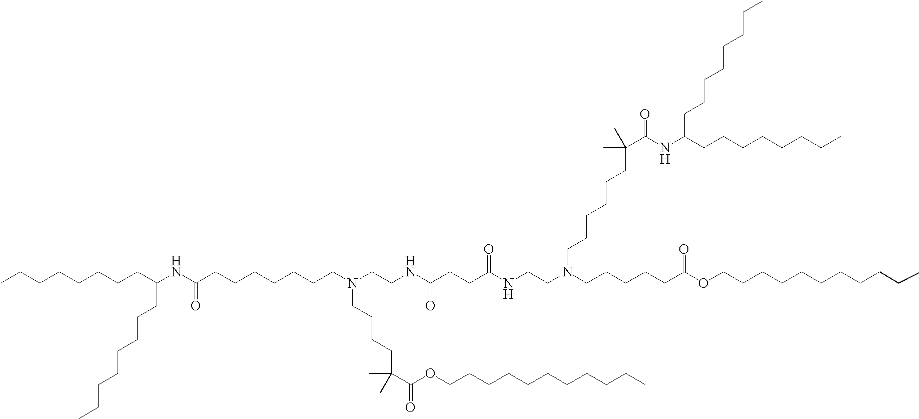 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | |
| A4 | 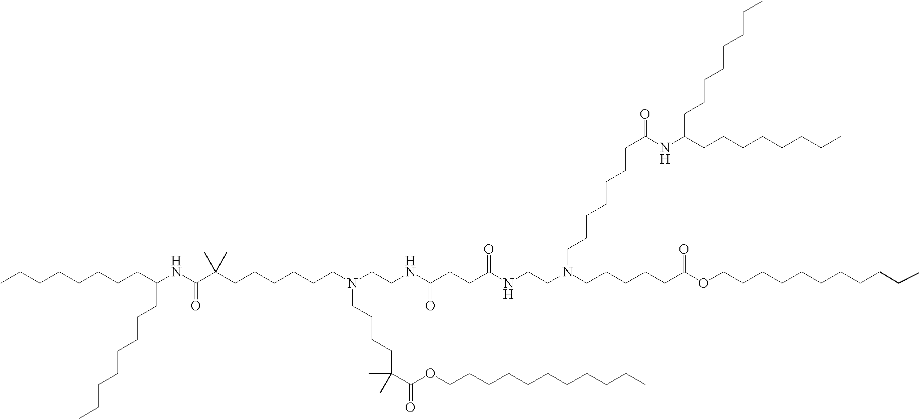 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] | |
| A5 | 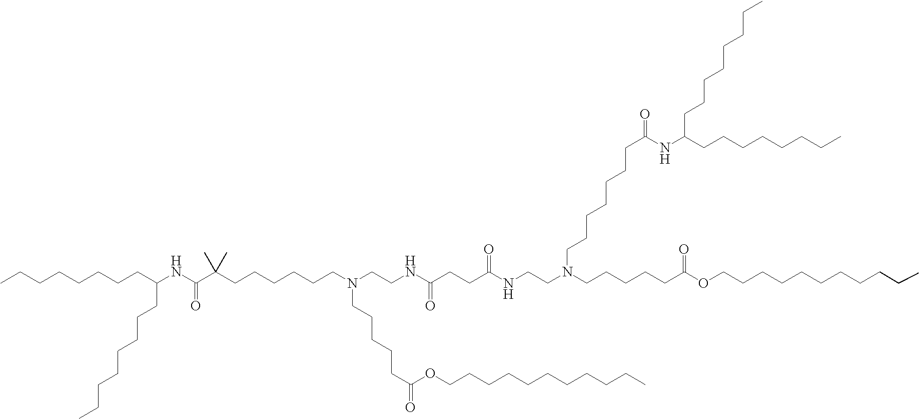 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OCCCCCCCCCCC] | |
| A6 | 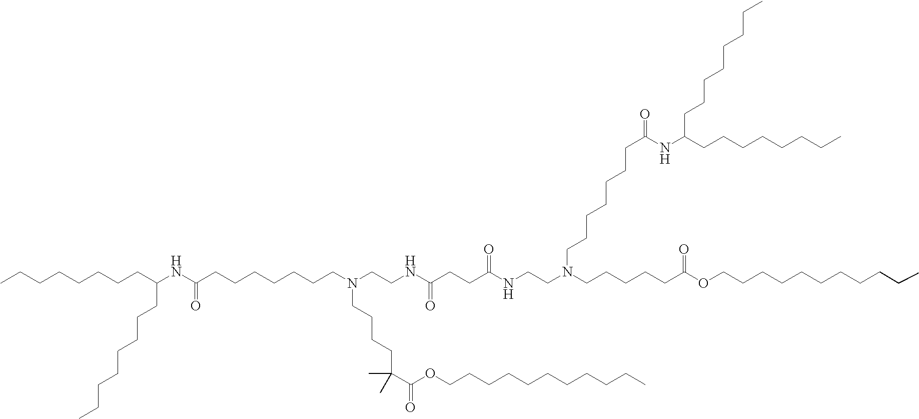 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] | |
| A7 | 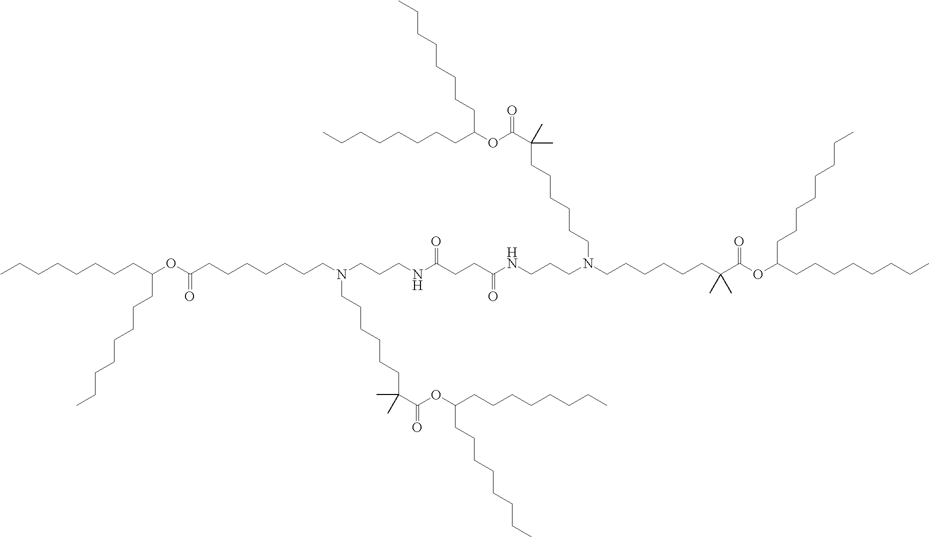 [SMILES: CCCCCCCCC(CCCCCCCC)OC(=O)CCCCCCCN(CCCCCCC(C)(C)C(=O)OC(CCCCCCCC)CCCCCCCC)CCCNC(=O)CCC(=O)NCCCN(CCCCCCC(C)(C)C(=O)OC(CCCCCCCC)CCCCCCCC)CCCCCCC(C)(C)C(=O)OC(CCCCCCCC)CCCCCCCC] | |
| A8 | 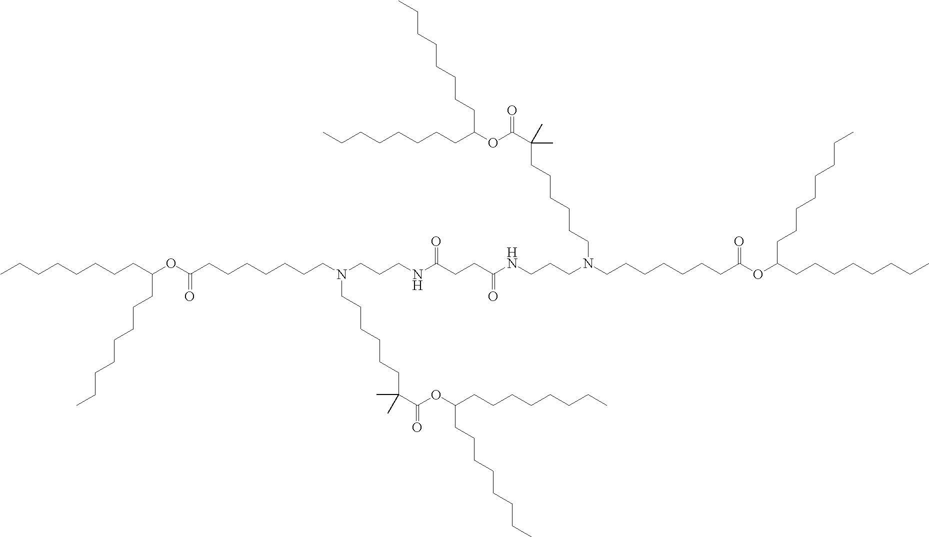 [SMILES: CCCCCCCCC(CCCCCCCC)OC(=O)CCCCCCCN(CCCCCCC(C)(C)C(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] | |
| A9 | 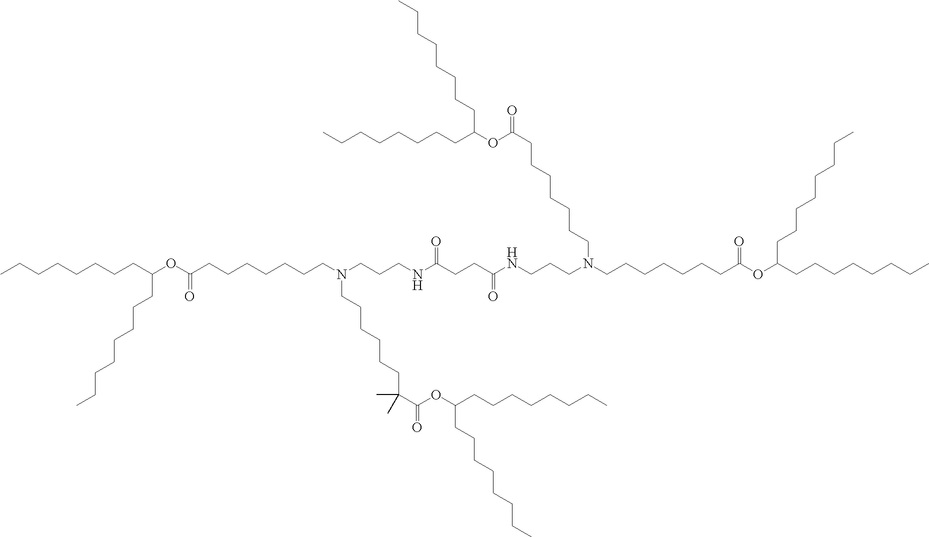 [SMILES: CCCCCCCCC(CCCCCCCC)OC(=O)CCCCCCCN(CCCCCCCC(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] | |
| A10 | 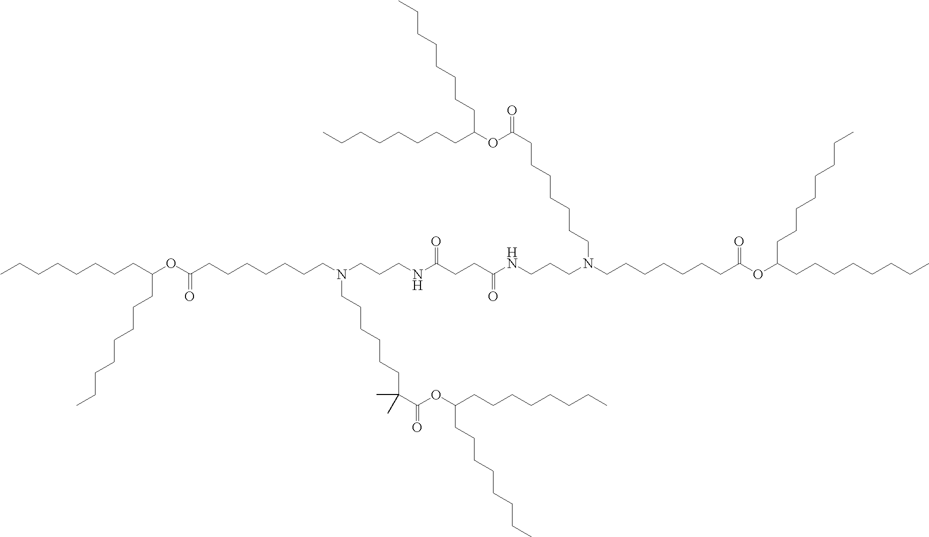 [SMILES: CCCCCCCCC(CCCCCCCC)OC(=O)CCCCCCCN(CCCCCCCC(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] | |
| A11 | 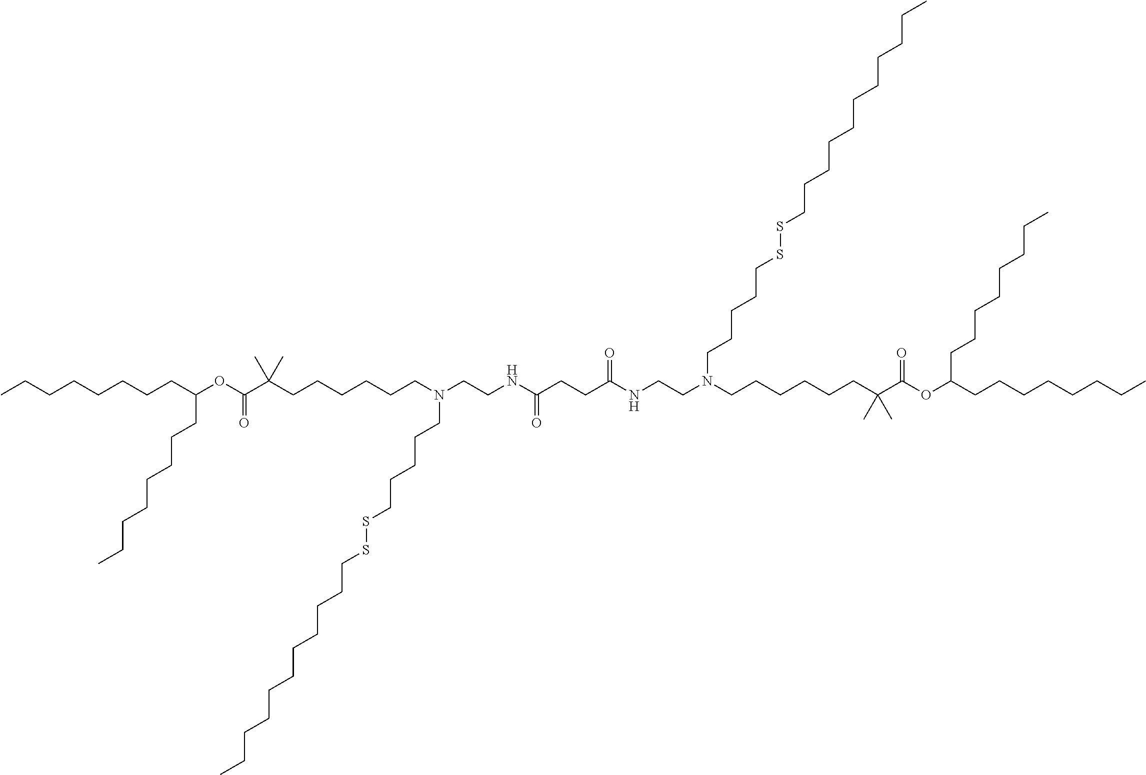 [SMILES: CCCCCCCCCCCSSCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCSSCCCCCCCCCCC] | |
| A12 | 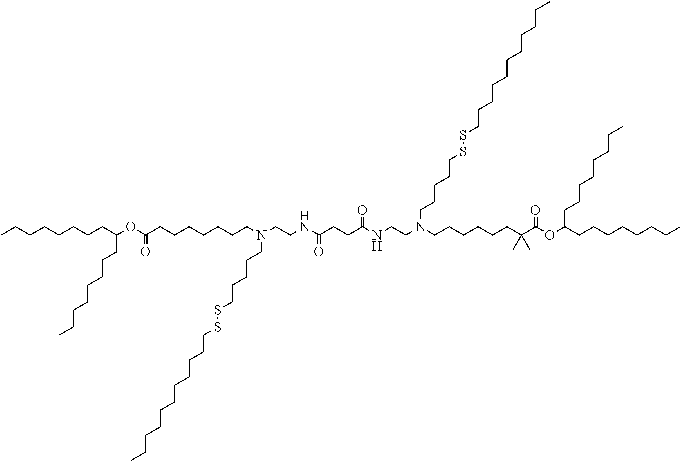 [SMILES: CCCCCCCCCCCSSCCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCSSCCCCCCCCCCC] | |
| A13 | 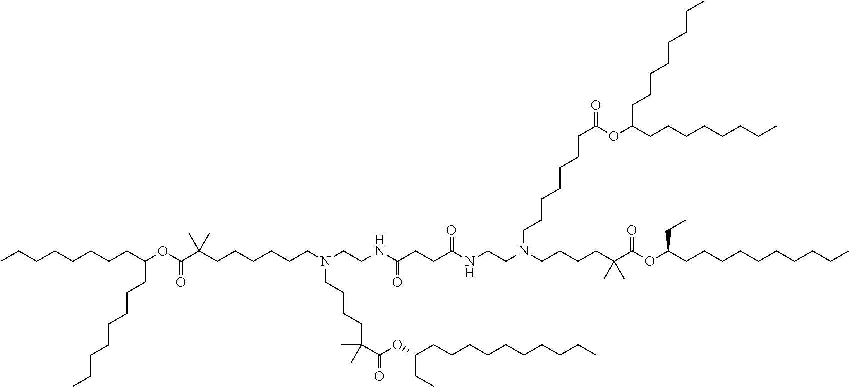 [SMILES: CCCCCCCCCC[C@H](CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] | |
| A14 | 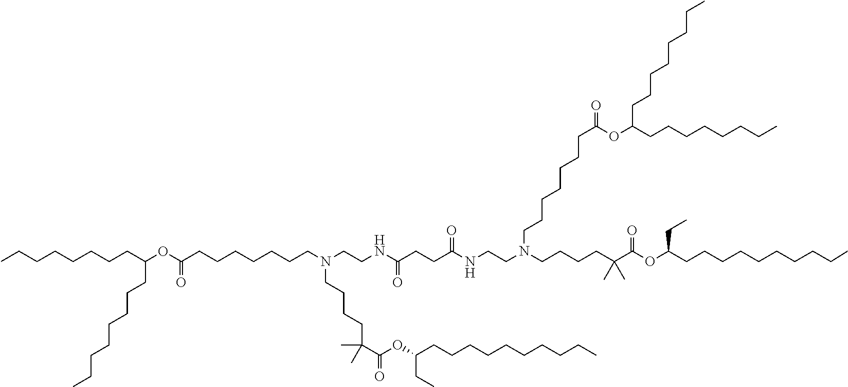 [SMILES: CCCCCCCCCC[C@H](CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | |
| A15 | 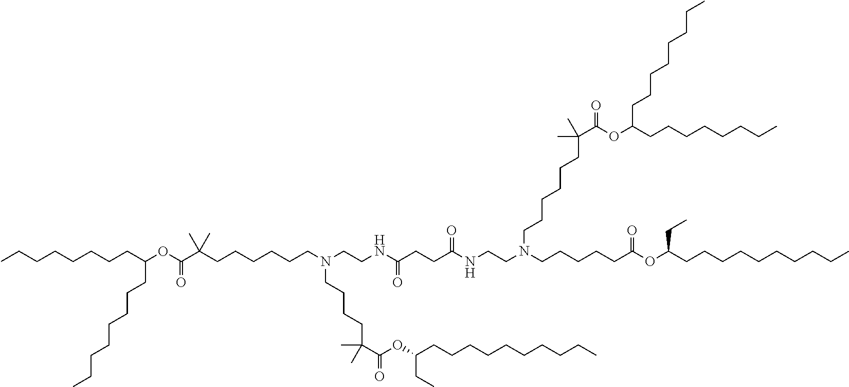 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] | |
| A16 | 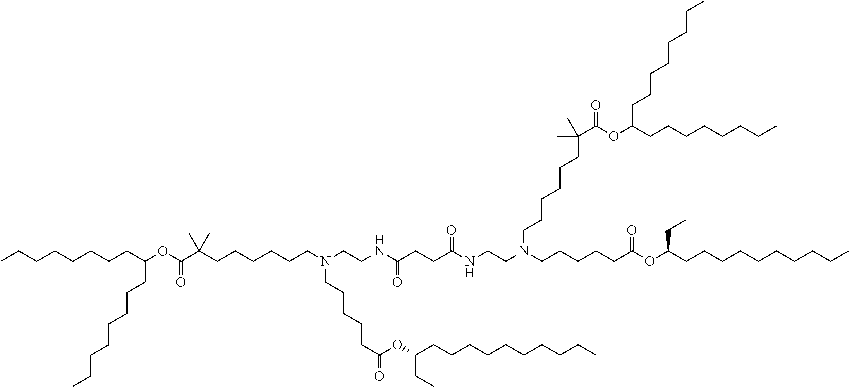 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@@H](CC)CCCCCCCCCC] | |
| A17 | 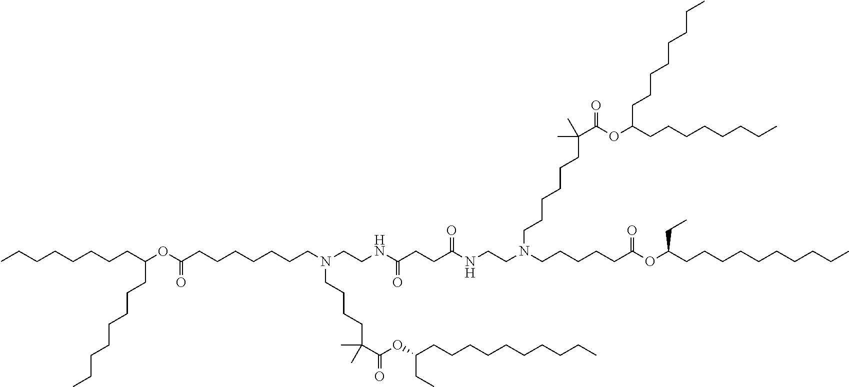 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | |
| A18 | 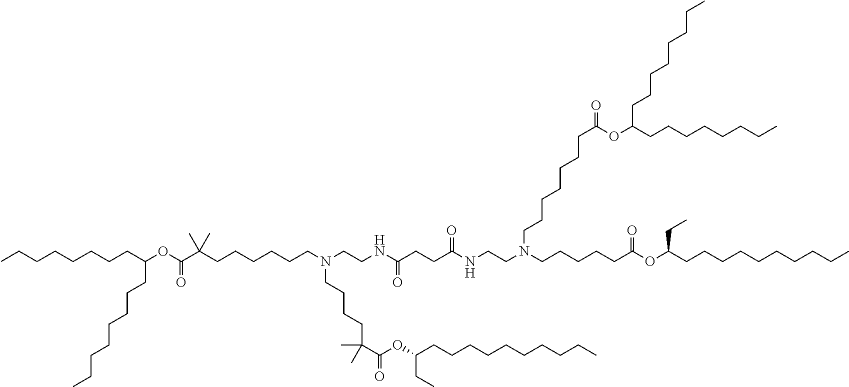 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] | |
| A19 | 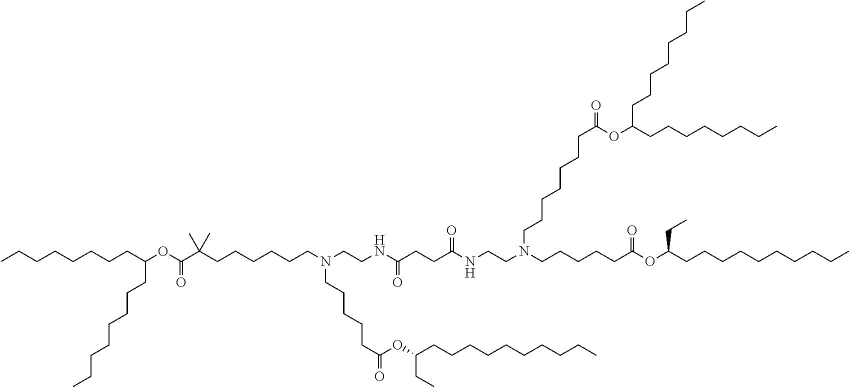 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@@H](CC)CCCCCCCCCC] | |
| A20 | 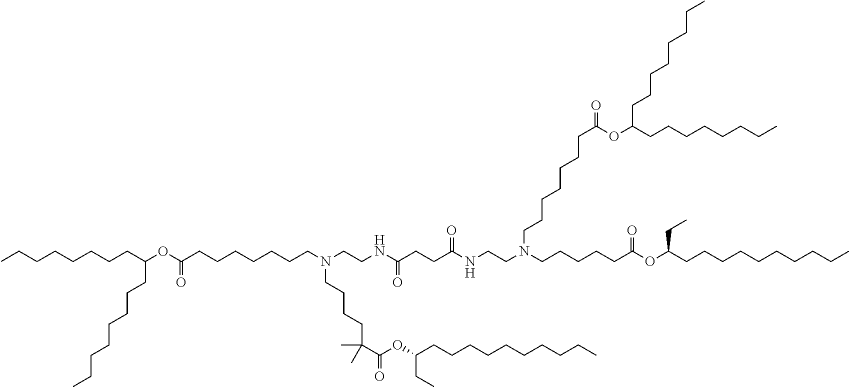 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] | |
| A21 | 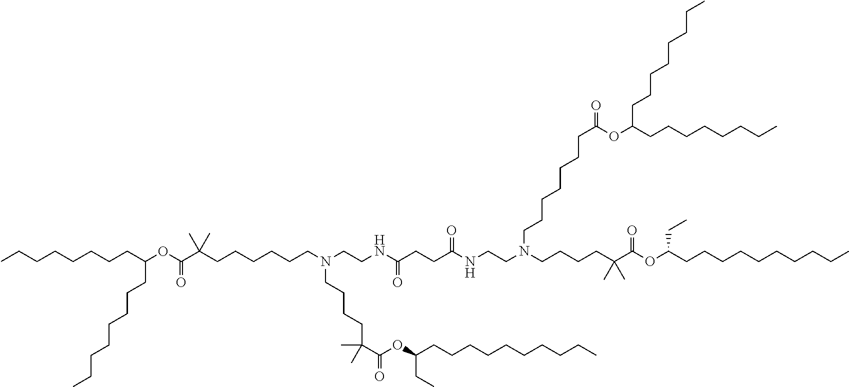 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | |
| A22 | 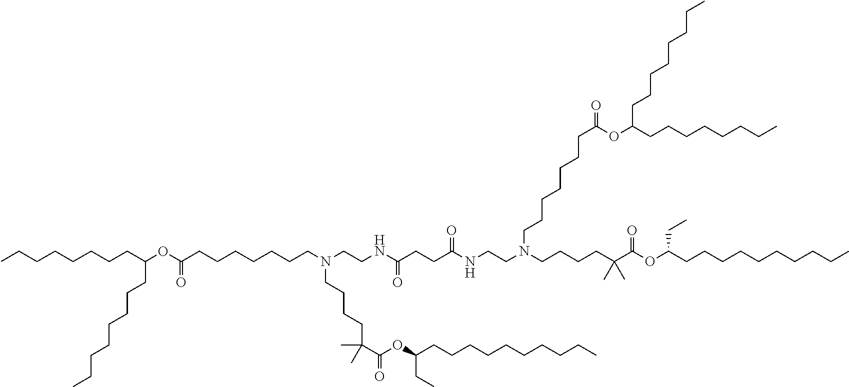 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] | |
| A23 | 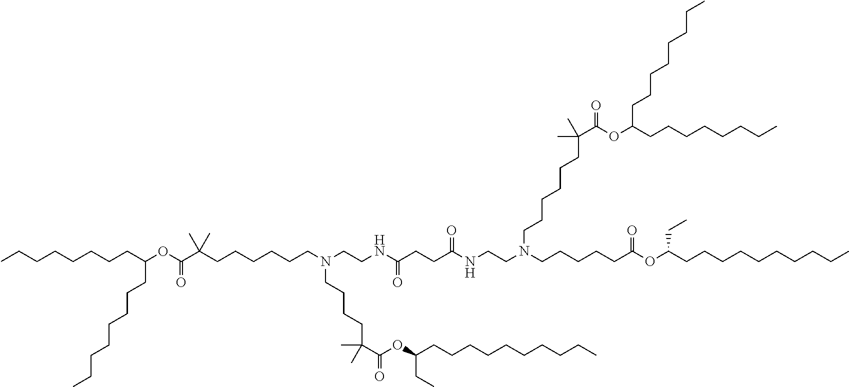 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | |
| A24 | 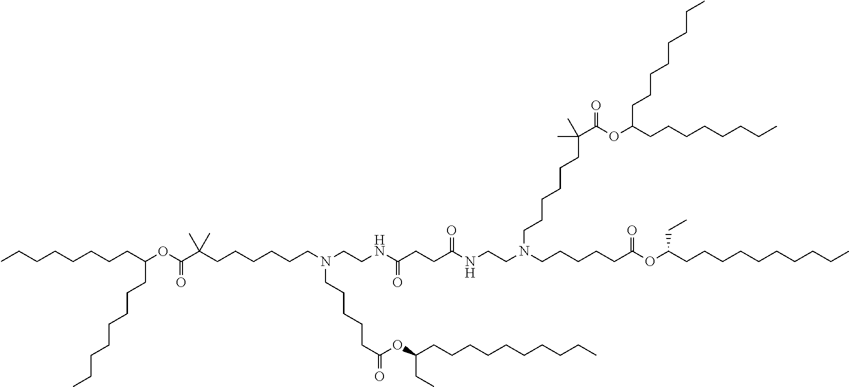 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@H](CC)CCCCCCCCCC] | |
| A25 | 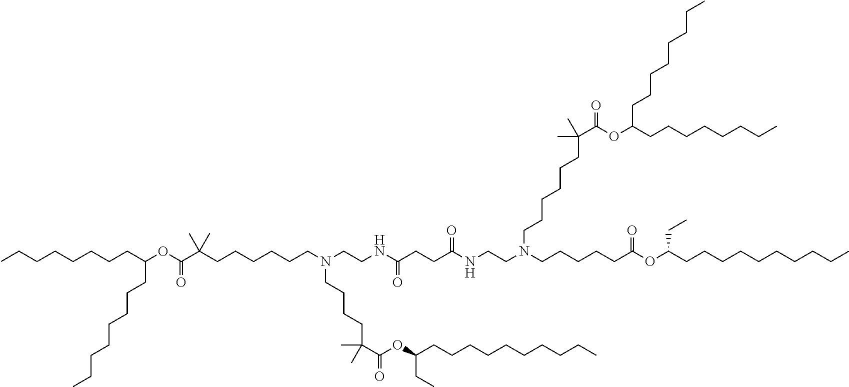 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | |
| A26 | 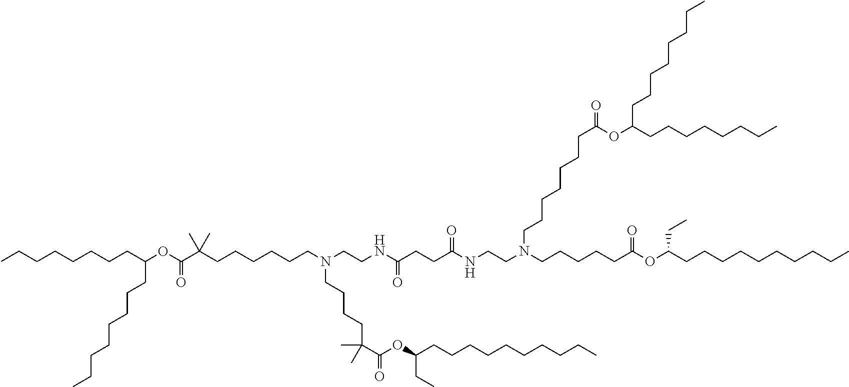 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] | |
| A27 | 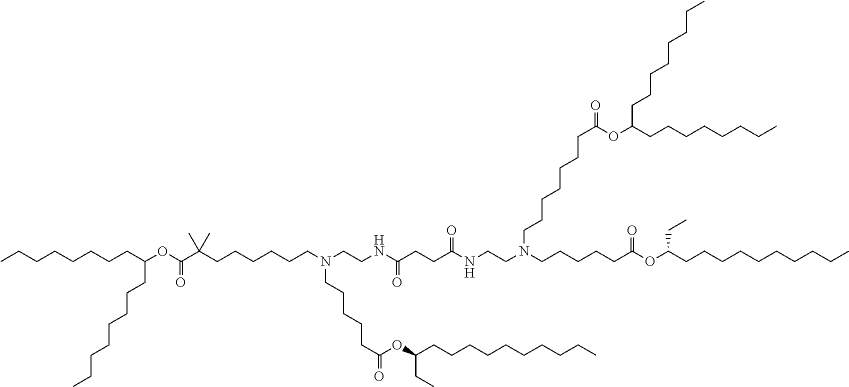 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@H](CC)CCCCCCCCCC] | |
| A28 | 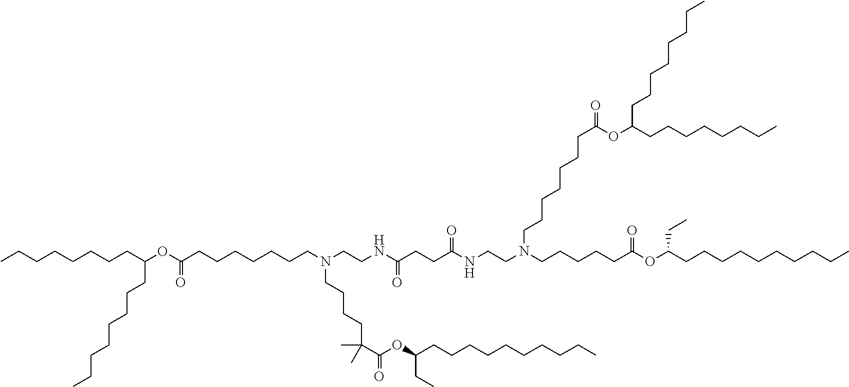 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] | |
| A29 | 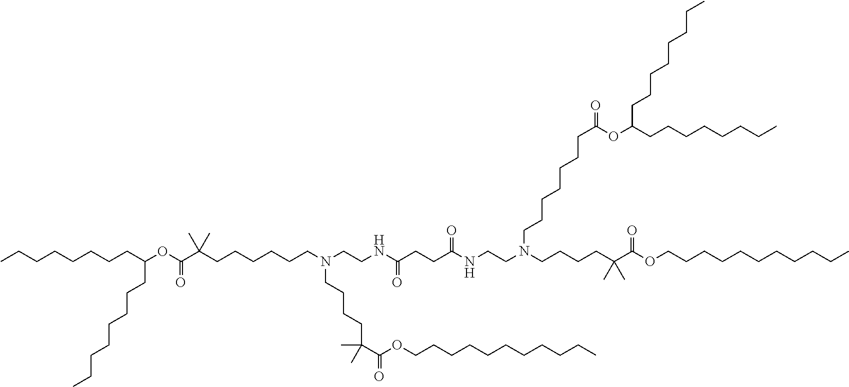 [SMILES: CCCCCCCCCCCOC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | |
| A30 | 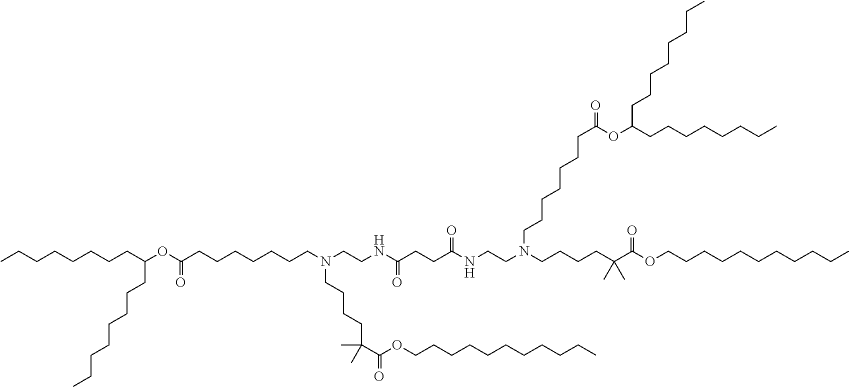 [SMILES: CCCCCCCCCCCOC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] | |
| A31 | 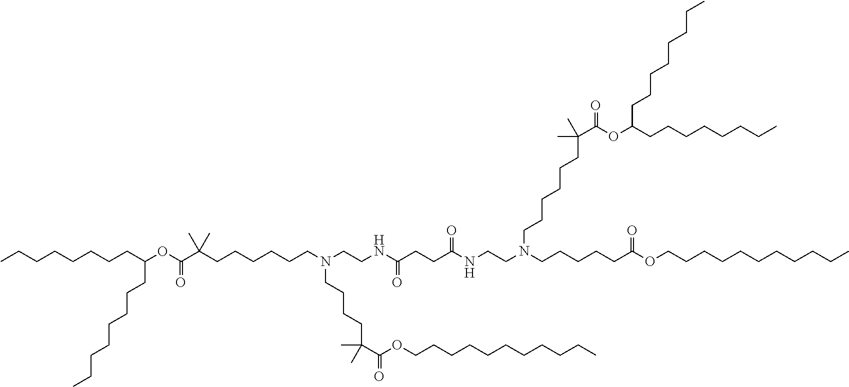 [SMILES: CCCCCCCCCCCOC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | |
| A32 | 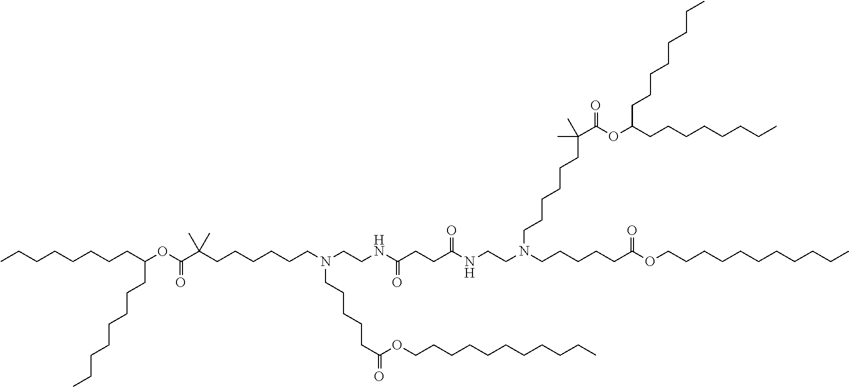 [SMILES: CCCCCCCCCCCOC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OCCCCCCCCCCC] | |
| A33 | 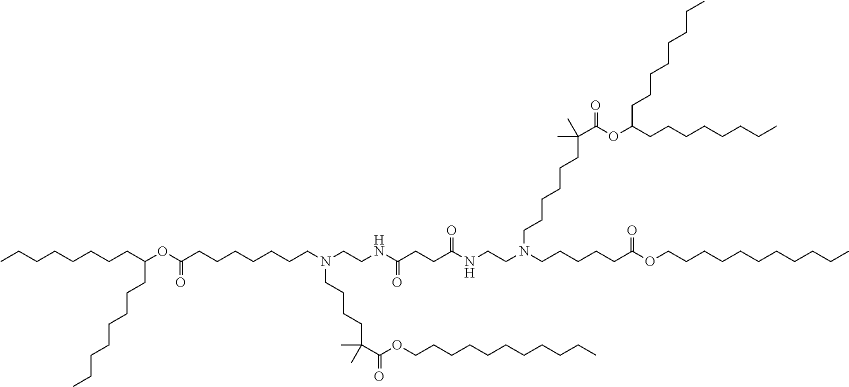 [SMILES: CCCCCCCCCCCOC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] | |
| A34 | 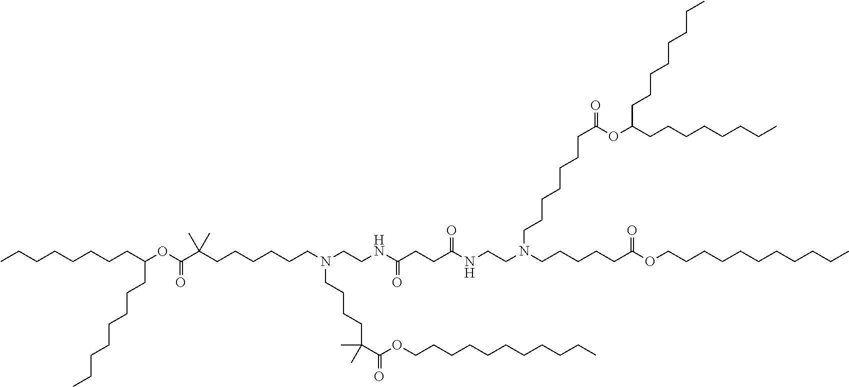 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] | |
| A35 | 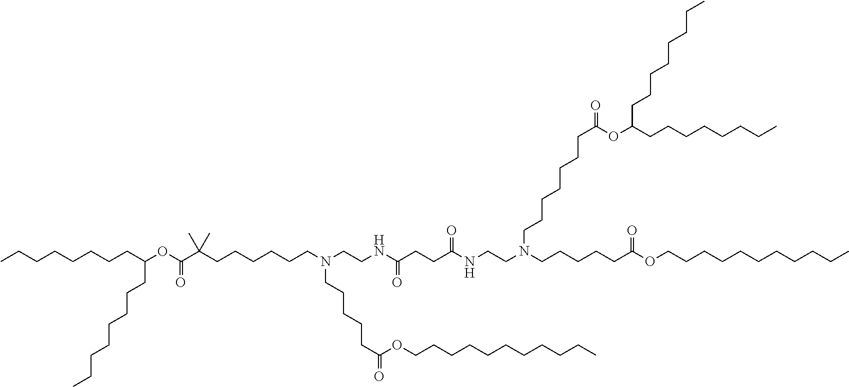 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OCCCCCCCCCCC] | |
| A36 | 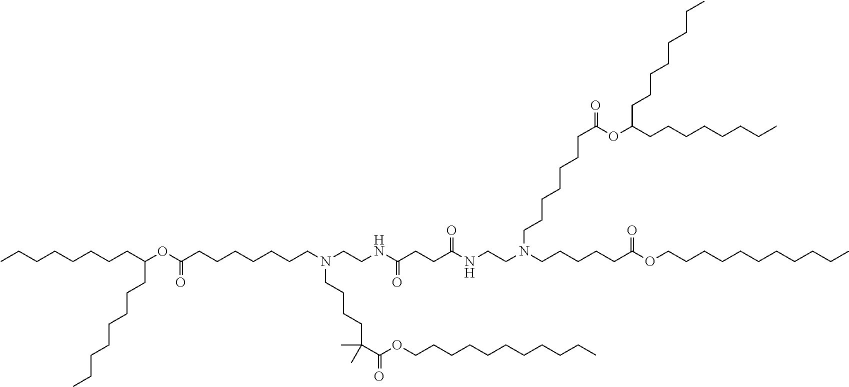 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] | |
| A37 | 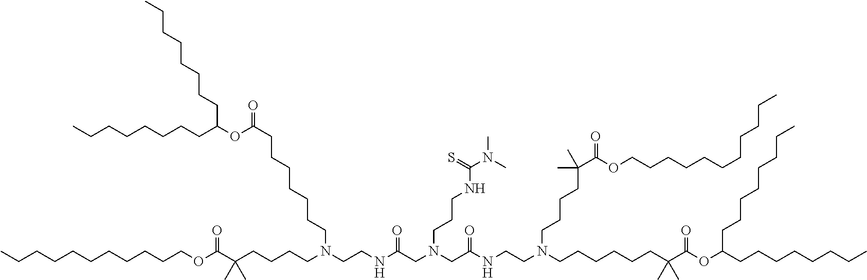 [SMILES: CCCCCCCCCCCOC(=O)C(C)(C)CCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] | |
| A38 | 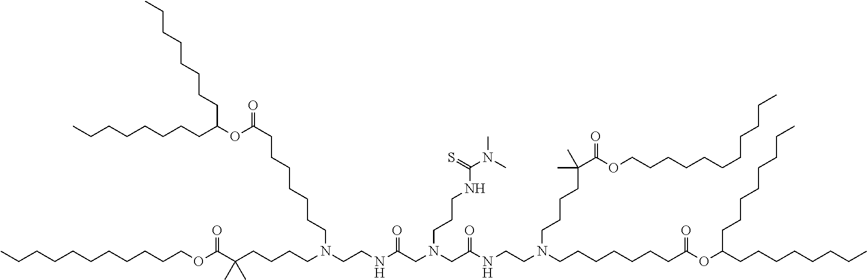 [SMILES: CCCCCCCCCCCOC(=O)C(C)(C)CCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] | |
| A39 | 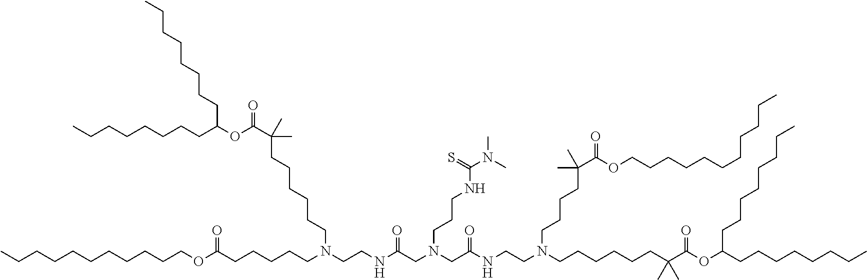 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] | |
| A40 | 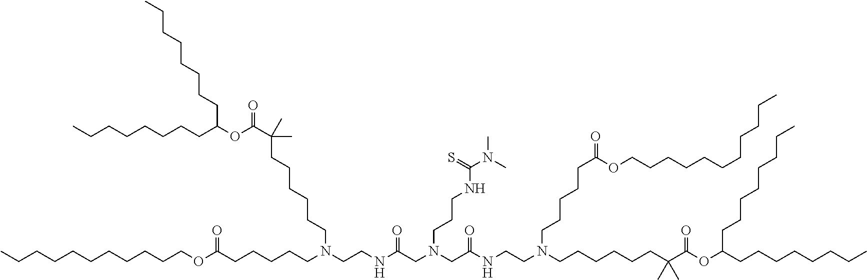 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCCC(=O)OCCCCCCCCCCC] | |
| A41 | 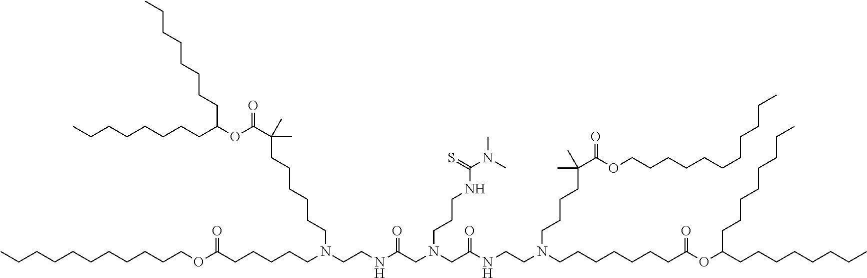 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] | |
| A42 | 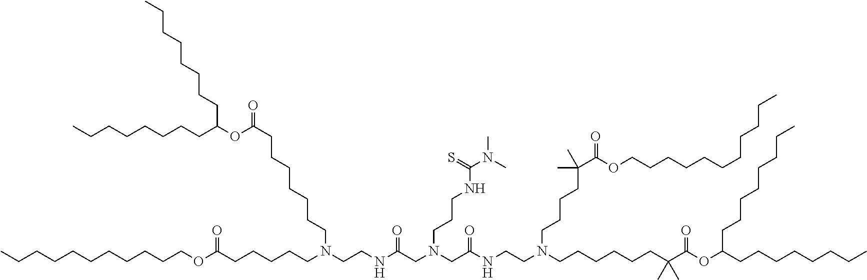 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] | |
| A43 | 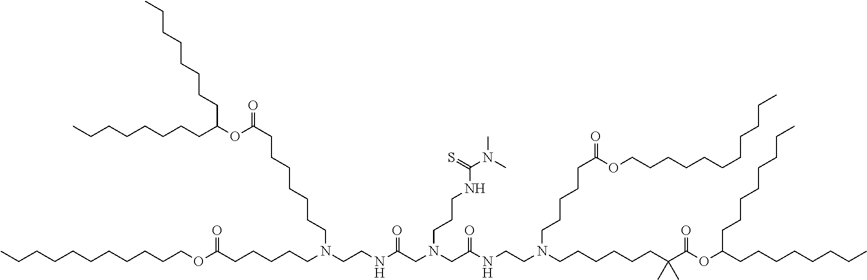 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCCC(=O)OCCCCCCCCCCC] | |
| A44 | 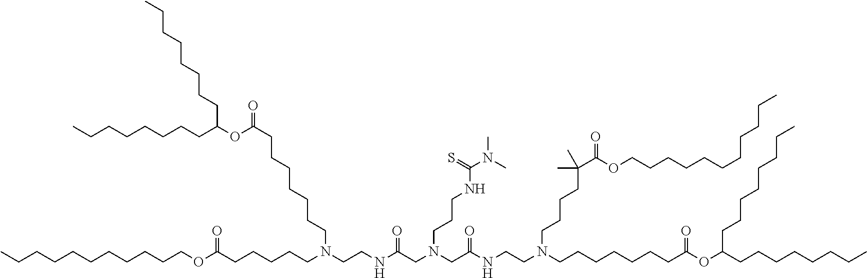 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] | |
| A45 | 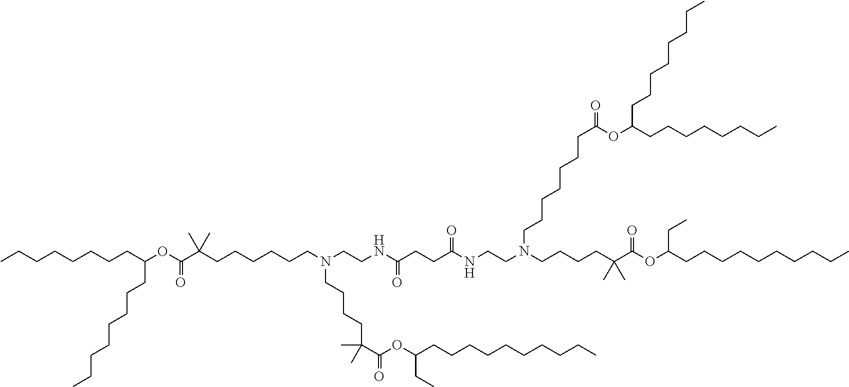 [SMILES: CCCCCCCCCCC(CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | |
| A46 | 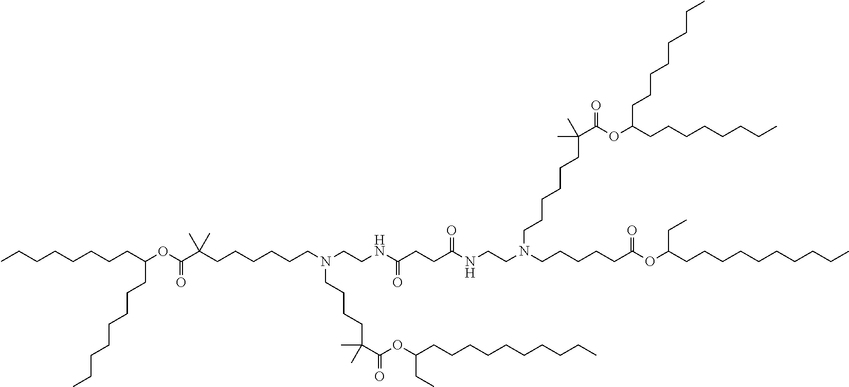 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | |
| A47 | 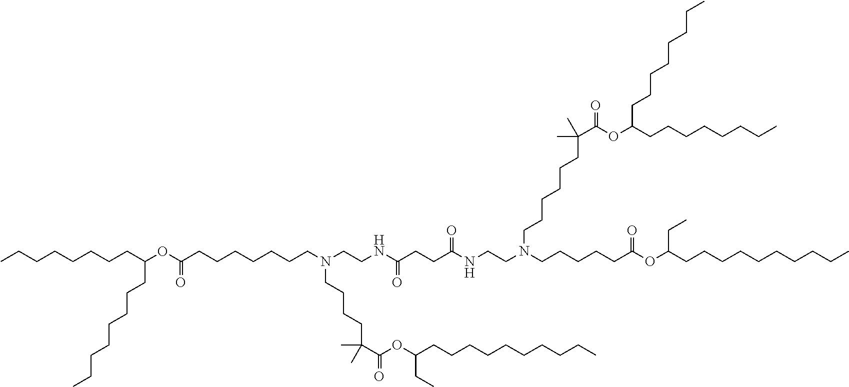 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] | |
| A48 | 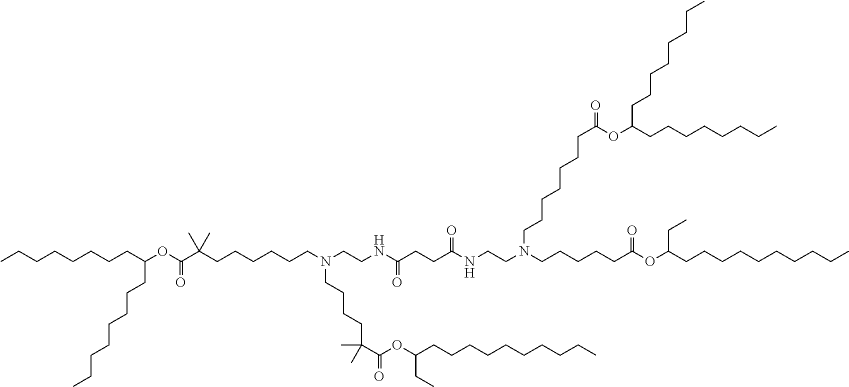 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] | |
| A49 | 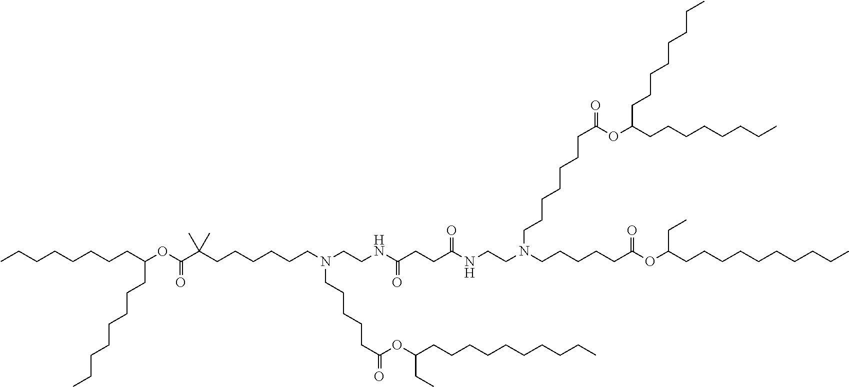 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OC(CC)CCCCCCCCCC] | |
| A50 | 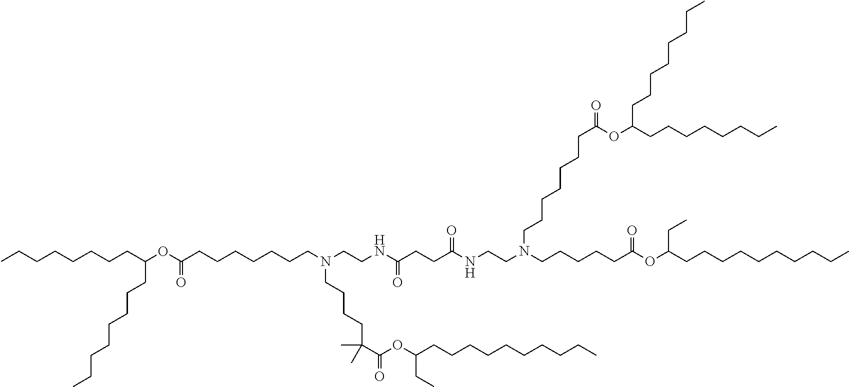 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] | |
| A51 | 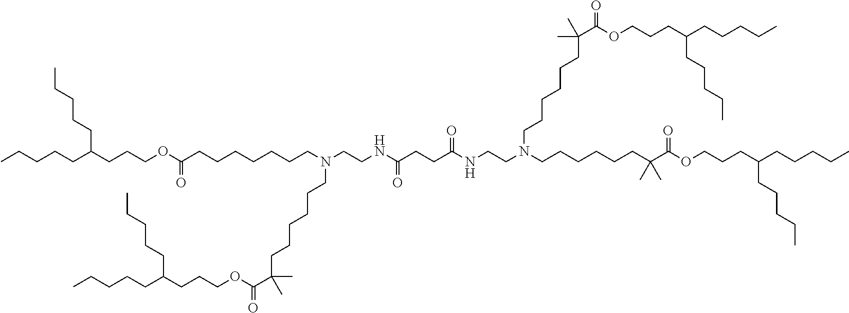 [SMILES: CCCCCC(CCCCC)CCCOC(=O)CCCCCCCN(CCCCCCC(C)(C)C(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] | |
| A52 | 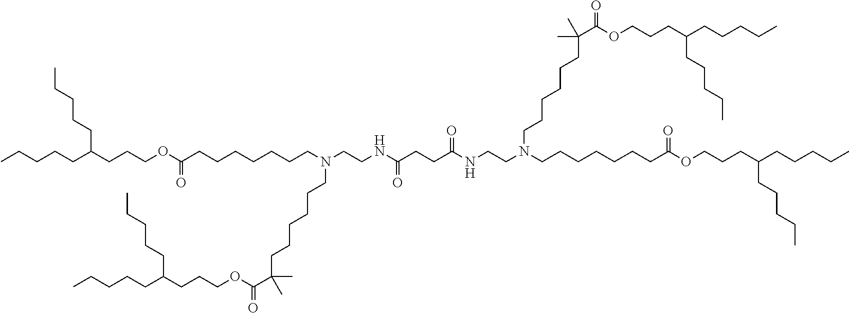 [SMILES: CCCCCC(CCCCC)CCCOC(=O)CCCCCCCN(CCCCCCC(C)(C)C(=O)OCCCC(CCCCC)CCCCC)CCNC(=O)CCC(=O)NCCN(CCCCCCCC(=O)OCCCC(CCCCC)CCCCC)CCCCCCC(C)(C)C(=O)OCCCC(CCCCC)CCCCC] | |
| A53 | 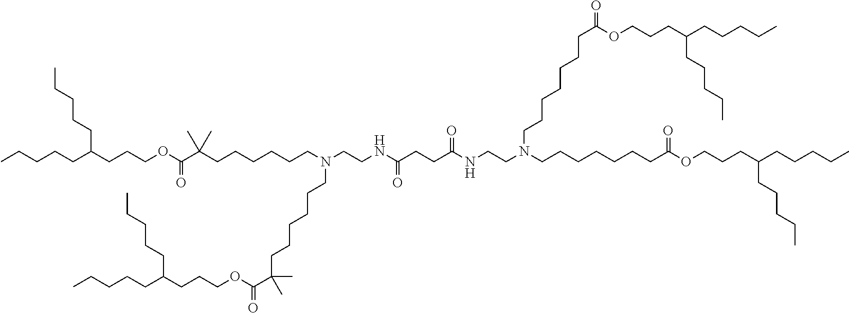 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] | |
| A54 | 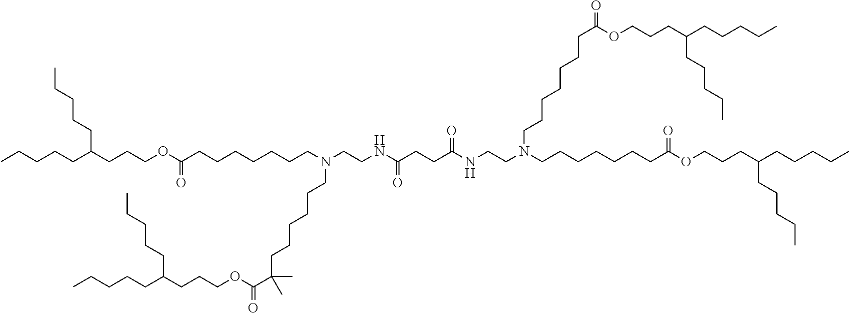 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCCC(=O)OCCCC(CCCCC)CCCCC)CCCCCCC(C)(C)C(=O)OCCCC(CCCCC)CCCCC] | |
| A55 | 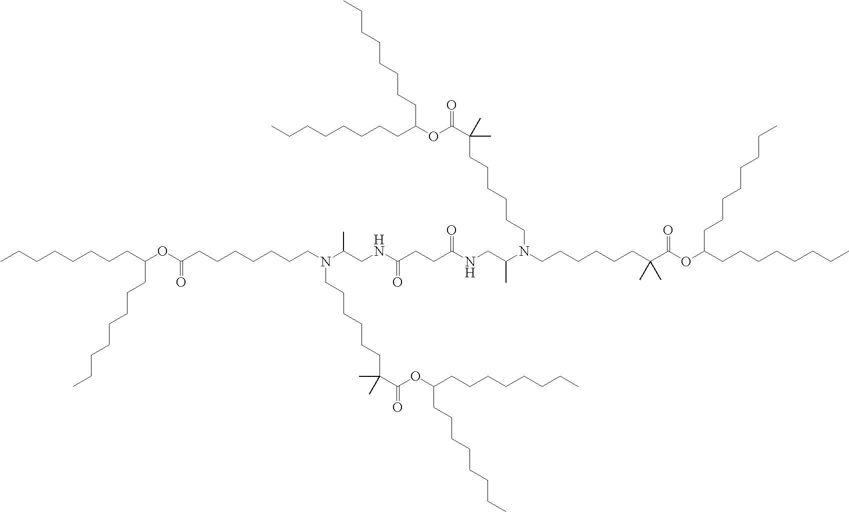 [SMILES: CCCCCCCCC(CCCCCCCC)OC(=O)CCCCCCCN(CCCCCCC(C)(C)C(=O)OC(CCCCCCCC)CCCCCCCC)C(C)CNC(=O)CCC(=O)NCC(C)N(CCCCCCC(C)(C)C(=O)OC(CCCCCCCC)CCCCCCCC)CCCCCCC(C)(C)C(=O)OC(CCCCCCCC)CCCCCCCC] | |
| A56 | 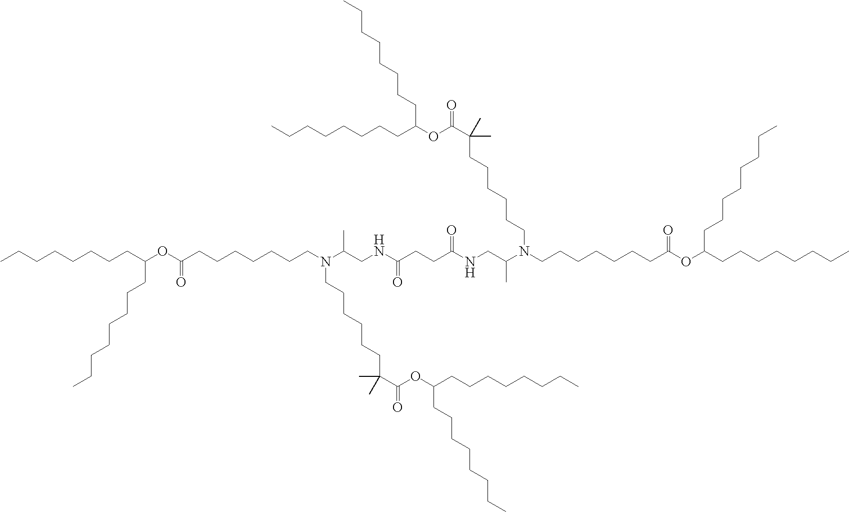 [SMILES: CCCCCCCCC(CCCCCCCC)OC(=O)CCCCCCCN(CCCCCCC(C)(C)C(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCC(C)(C)C(=O)OC(CCCCCCCC)CCCCCCCC] | |
| A57 | 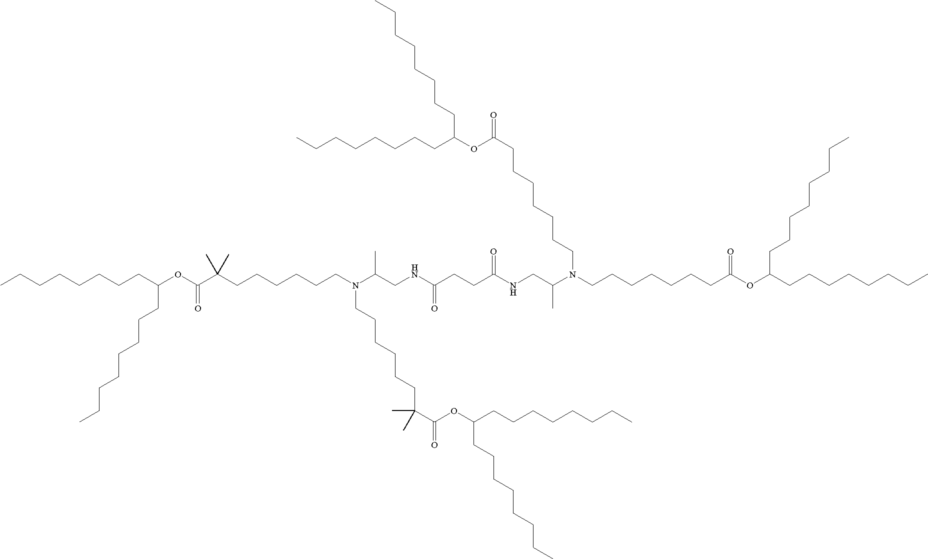 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCC(C)N(CCCCCCC(C)(C)C(=O)OC(CCCCCCCC)CCCCCCCC)CCCCCCC(C)(C)C(=O)OC(CCCCCCCC)CCCCCCCC] | |
| A58 | 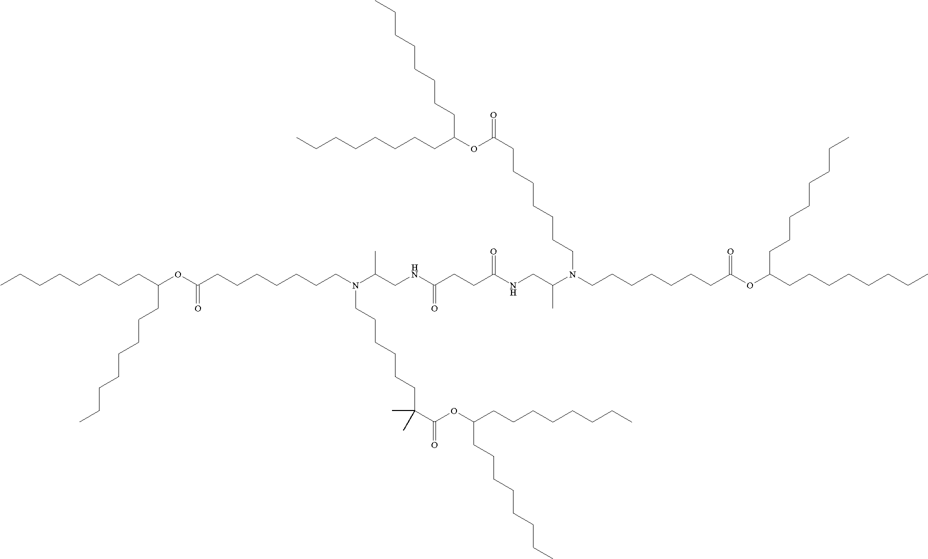 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCC(C)(C)C(=O)OC(CCCCCCCC)CCCCCCCC] | |
| A59 | 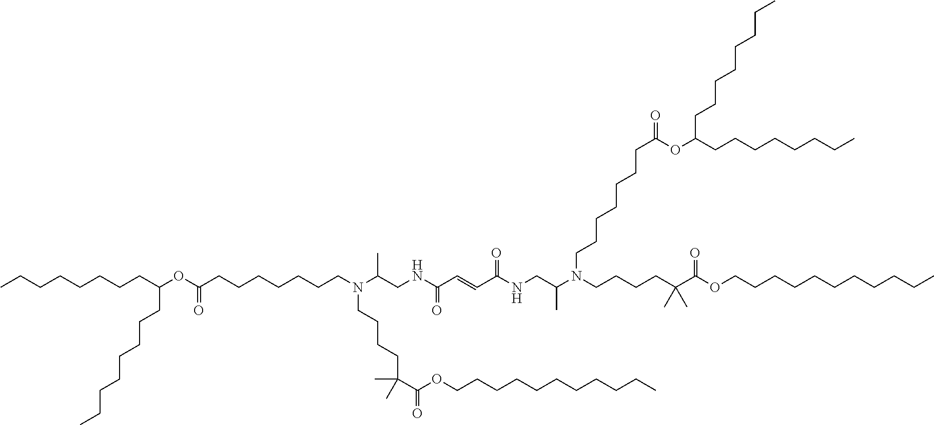 [SMILES: CCCCCCCCCCCOC(=O)C(C)(C)CCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] | |
| A60 | 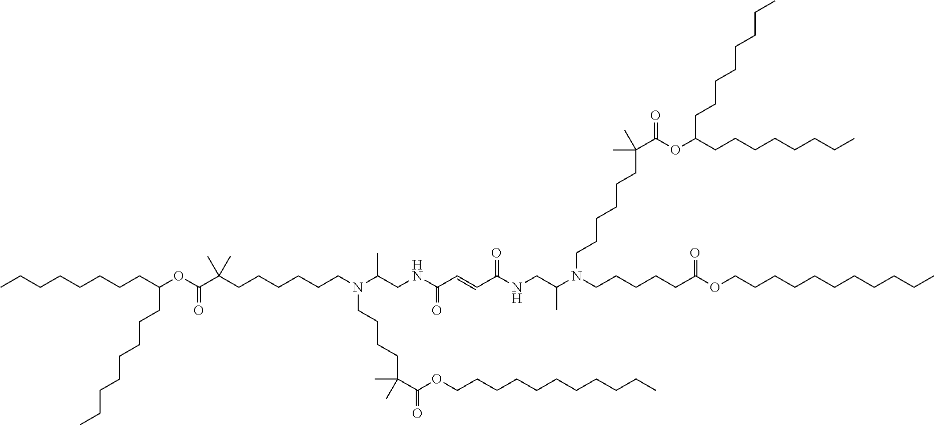 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCC(C)(C)C(=O)OCCCCCCCCCCC] | |
| A61 | 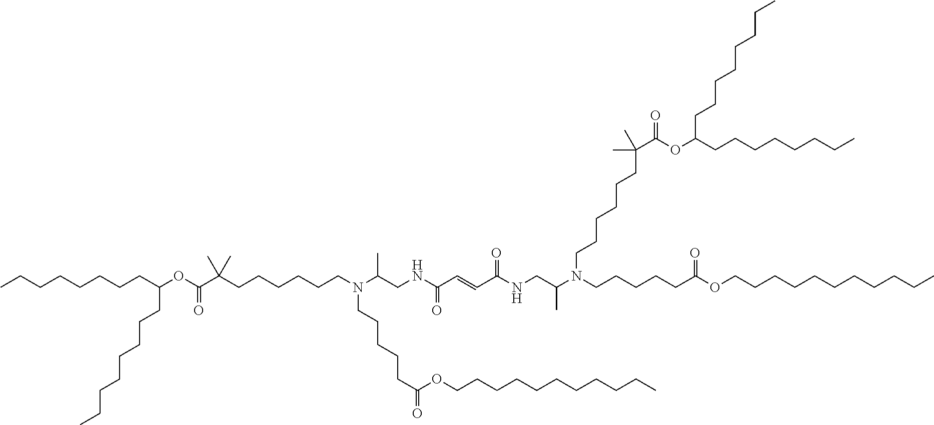 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCCC(=O)OCCCCCCCCCCC] | |
| A62 | 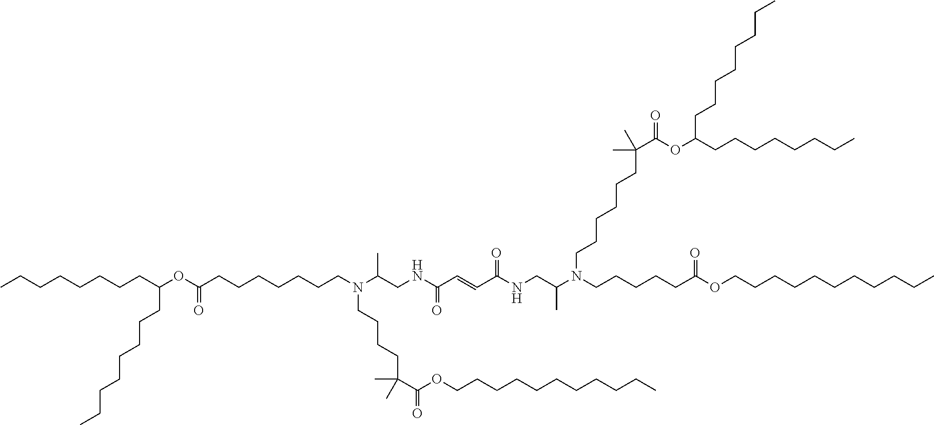 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] | |
| A63 | 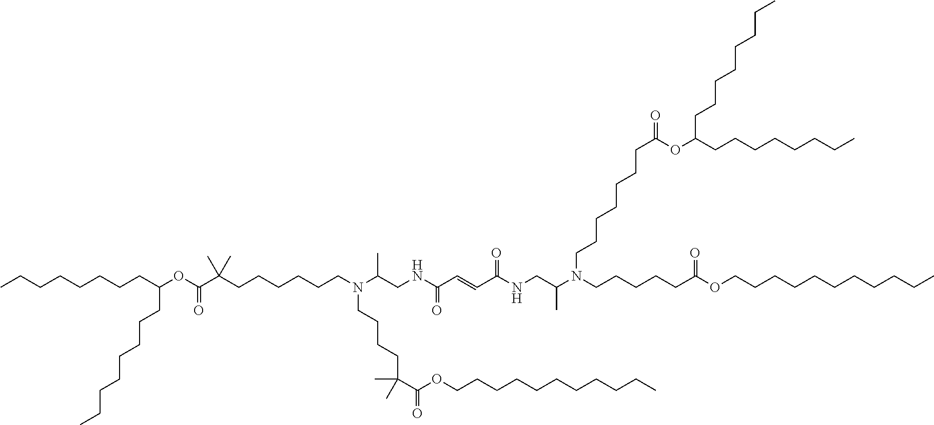 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCC(C)(C)C(=O)OCCCCCCCCCCC] | |
| A64 | 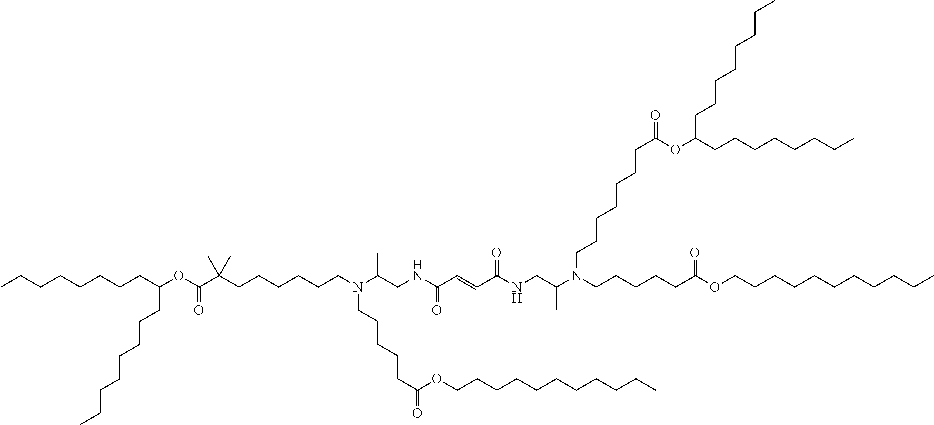 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCCC(=O)OCCCCCCCCCCC] | |
| A65 | 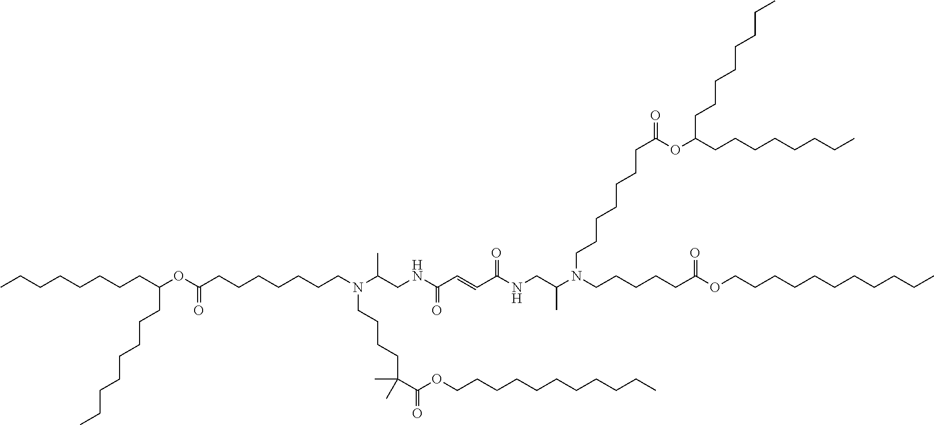 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] | |
| A66 | 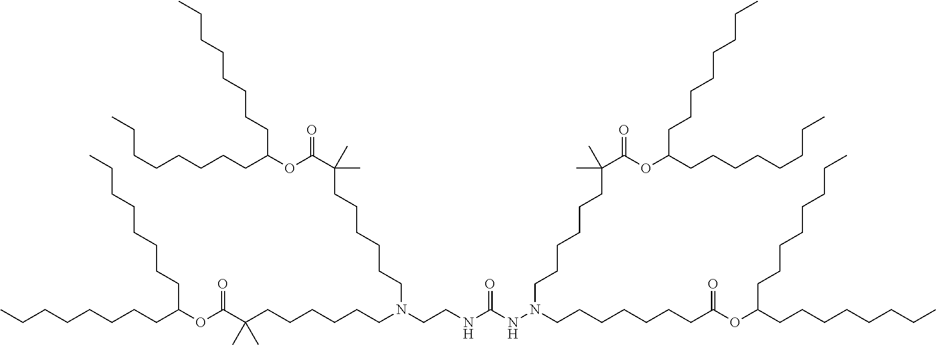 [SMILES: CCCCCCCCC(CCCCCCCC)OC(=O)CCCCCCCN(CCCCCCC(C)(C)C(=O)OC(CCCCCCCC)CCCCCCCC)NC(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A67 | 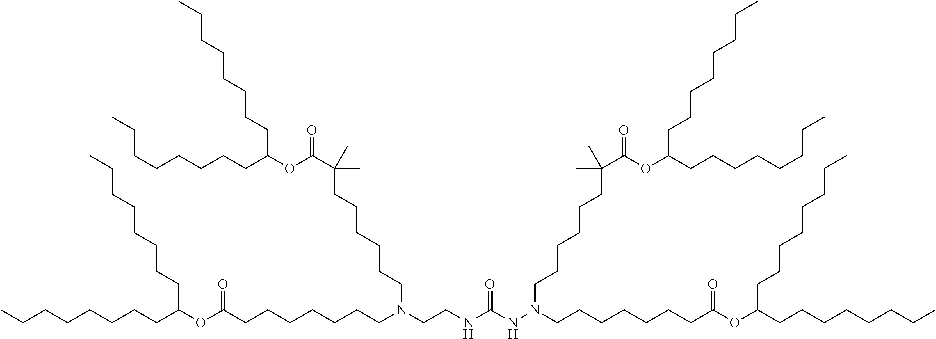 [SMILES: CCCCCCCCC(CCCCCCCC)OC(=O)CCCCCCCN(CCCCCCC(C)(C)C(=O)OC(CCCCCCCC)CCCCCCCC)CCNC(=O)NN(CCCCCCCC(=O)OC(CCCCCCCC)CCCCCCCC)CCCCCCC(C)(C)C(=O)OC(CCCCCCCC)CCCCCCCC] | |
| A68 | 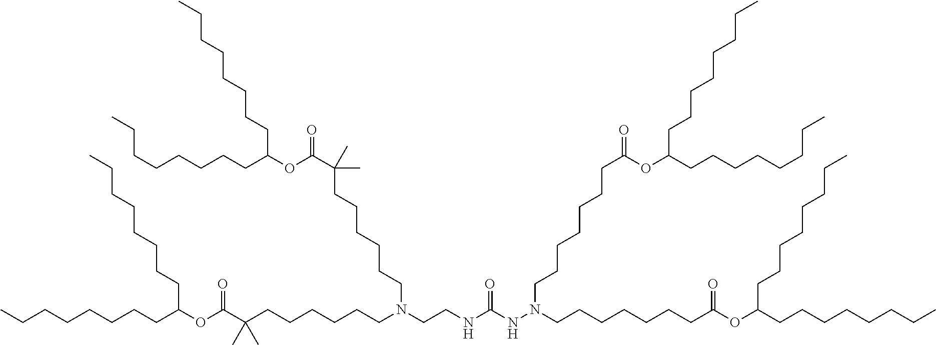 [SMILES: CCCCCCCCC(CCCCCCCC)OC(=O)CCCCCCCN(CCCCCCCC(=O)OC(CCCCCCCC)CCCCCCCC)NC(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A69 | 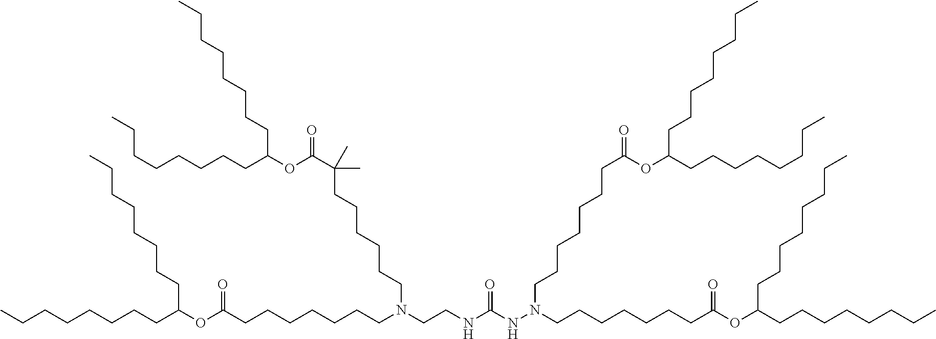 [SMILES: CCCCCCCCC(CCCCCCCC)OC(=O)CCCCCCCN(CCCCCCC(C)(C)C(=O)OC(CCCCCCCC)CCCCCCCC)CCNC(=O)NN(CCCCCCCC(=O)OC(CCCCCCCC)CCCCCCCC)CCCCCCCC(=O)OC(CCCCCCCC)CCCCCCCC] | |
| A70 | 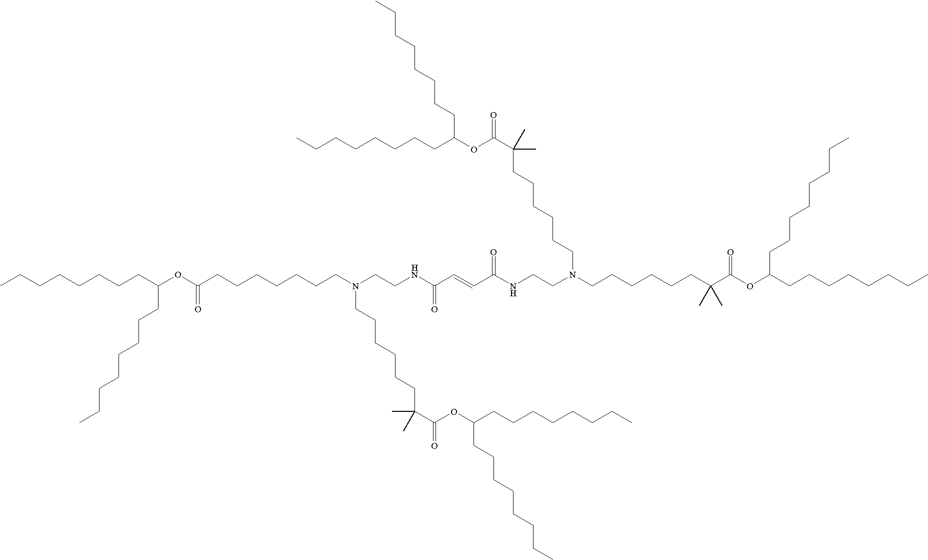 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A71 | 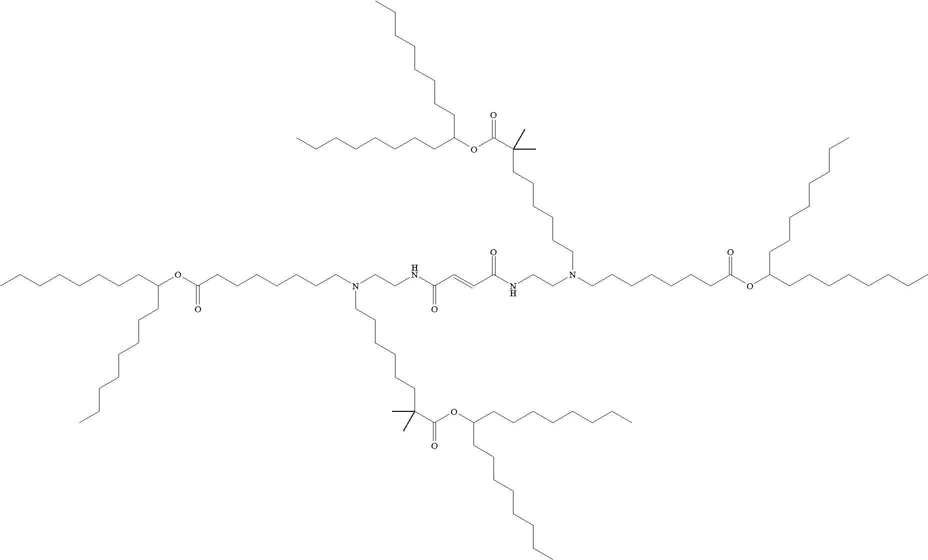 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCCCC(C)(C)C(=O)OC(CCCCCCCC)CCCCCCCC] | |
| A72 | 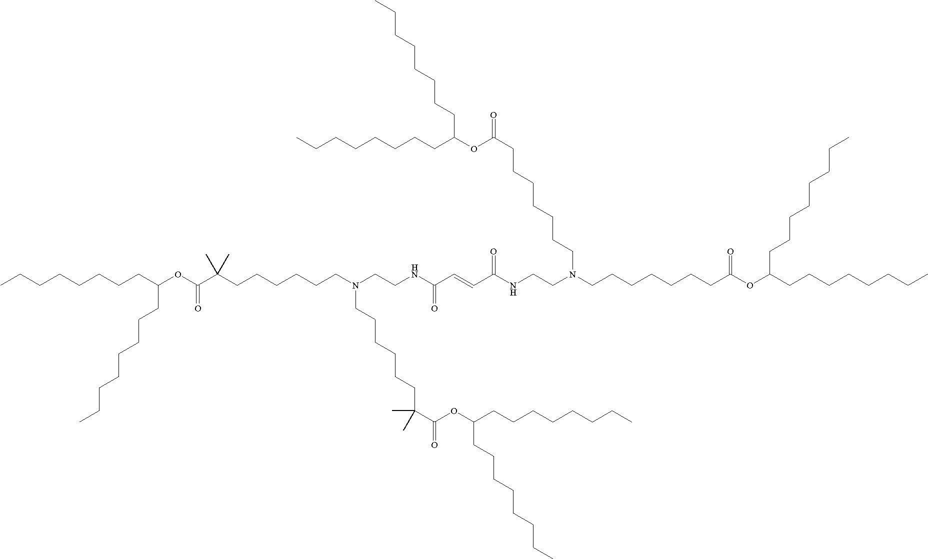 [SMILES: CCCCCCCCC(CCCCCCCC)OC(=O)CCCCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A73 | 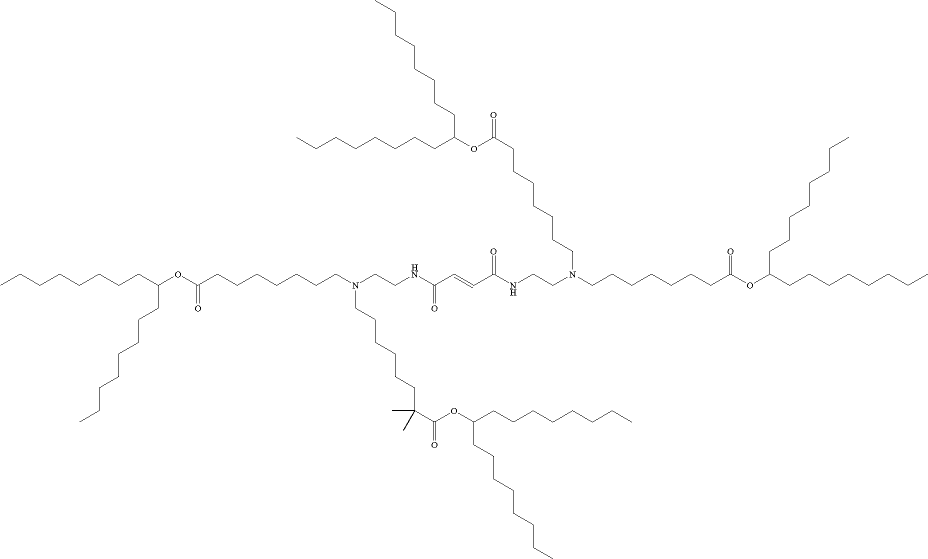 [SMILES: CCCCCCCCC(CCCCCCCC)OC(=O)CCCCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCCC(C)(C)C(=O)OC(CCCCCCCC)CCCCCCCC] | |
| A74 | 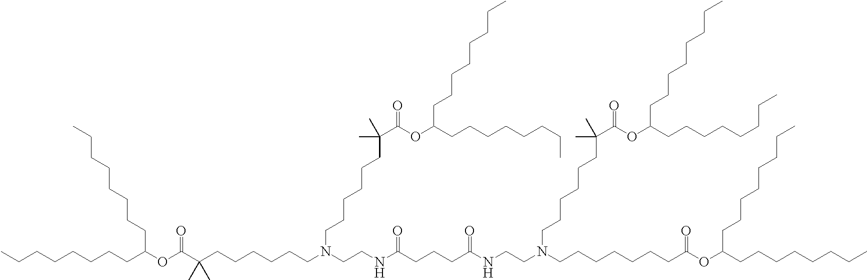 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A75 | 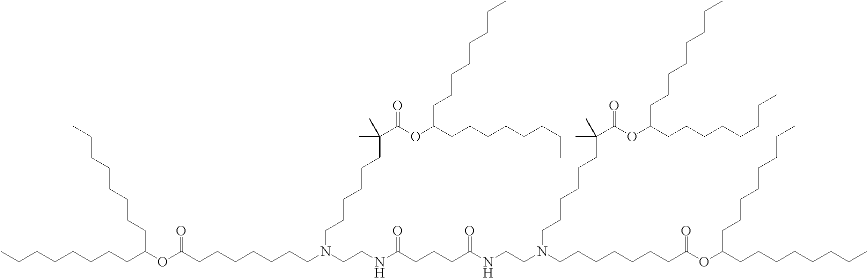 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCCC(=O)NCCN(CCCCCCCC(=O)OC(CCCCCCCC)CCCCCCCC)CCCCCCC(C)(C)C(=O)OC(CCCCCCCC)CCCCCCCC] | |
| A76 | 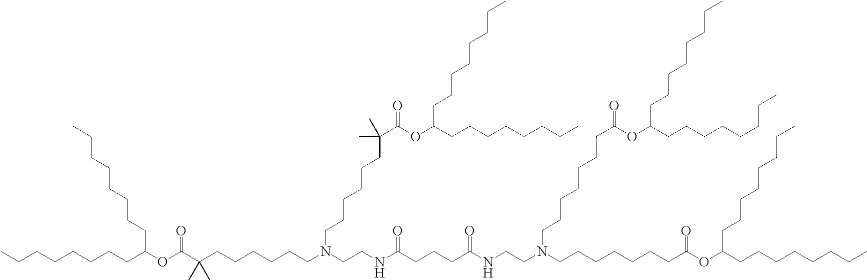 [SMILES: CCCCCCCCC(CCCCCCCC)OC(=O)CCCCCCCN(CCCCCCCC(=O)OC(CCCCCCCC)CCCCCCCC)CCNC(=O)CCCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A77 | 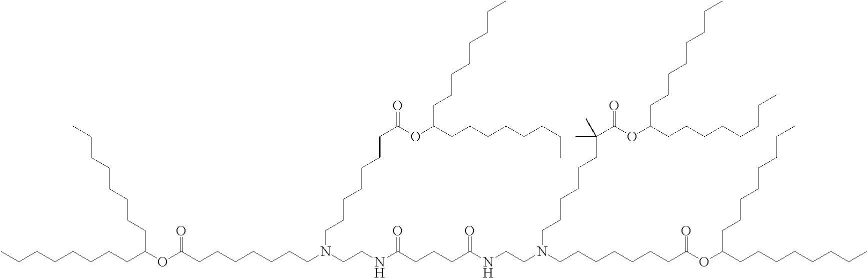 [SMILES: CCCCCCCCC(CCCCCCCC)OC(=O)CCCCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCCC(C)(C)C(=O)OC(CCCCCCCC)CCCCCCCC] | |
| A78 | 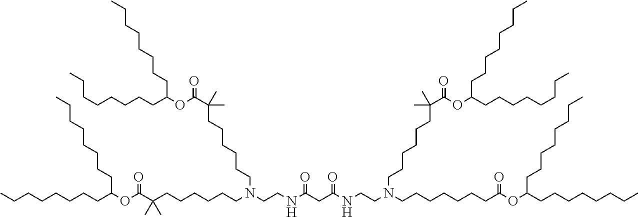 [SMILES: CCCCCCCCC(CCCCCCCC)OC(=O)CCCCCCCN(CCCCCCC(C)(C)C(=O)OC(CCCCCCCC)CCCCCCCC)CCNC(=O)CC(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A79 | 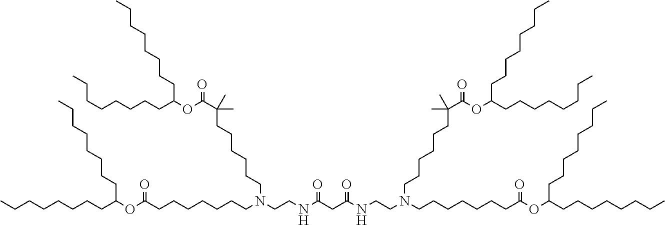 [SMILES: CCCCCCCCC(CCCCCCCC)OC(=O)CCCCCCCN(CCCCCCC(C)(C)C(=O)OC(CCCCCCCC)CCCCCCCC)CCNC(=O)CC(=O)NCCN(CCCCCCCC(=O)OC(CCCCCCCC)CCCCCCCC)CCCCCCC(C)(C)C(=O)OC(CCCCCCCC)CCCCCCCC] | |
| A80 | 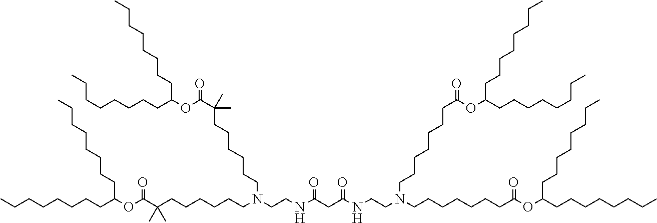 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A81 | 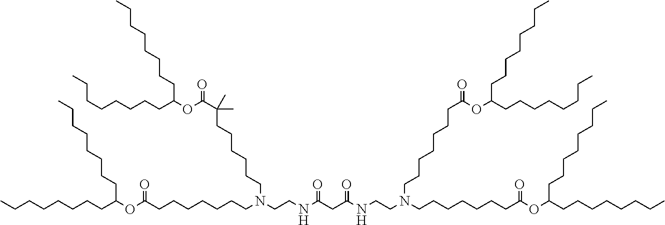 [SMILES: CCCCCCCCC(CCCCCCCC)OC(=O)CCCCCCCN(CCCCCCCC(=O)OC(CCCCCCCC)CCCCCCCC)CCNC(=O)CC(=O)NCCN(CCCCCCCC(=O)OC(CCCCCCCC)CCCCCCCC)CCCCCCC(C)(C)C(=O)OC(CCCCCCCC)CCCCCCCC] | |
| A82 | 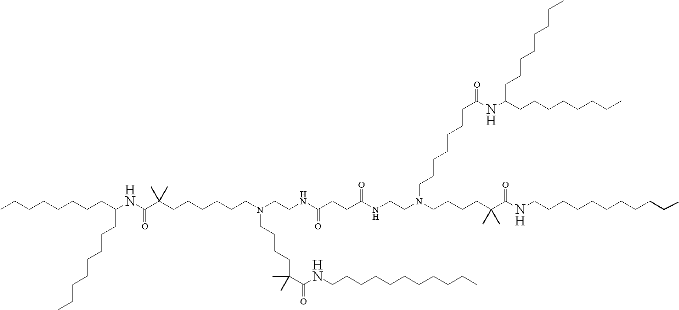 [SMILES: CCCCCCCCCCCNC(=O)C(C)(C)CCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] | |
| A83 | 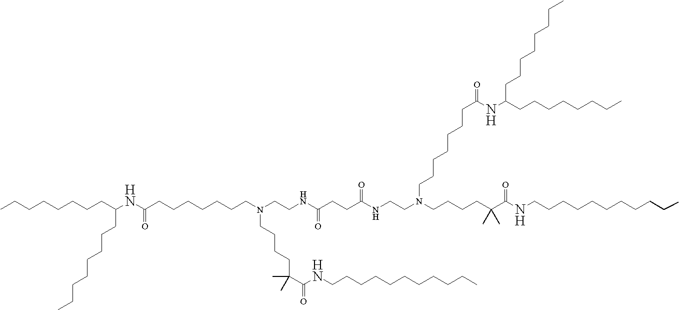 [SMILES: CCCCCCCCCCCNC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)NCCCCCCCCCCC] | |
| A84 | 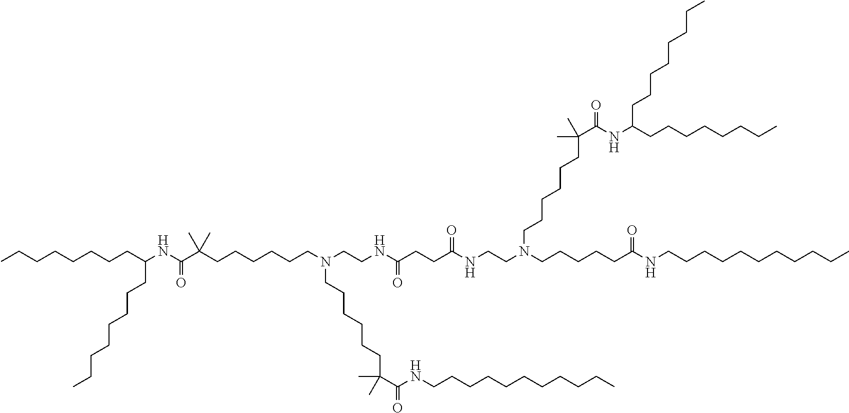 [SMILES: CCCCCCCCCCCNC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NCCCCCCCCCCC)CCCCCCC(C)(C)C(=O)NC(CCCCCCCC)CCCCCCCC] | |
| A85 | 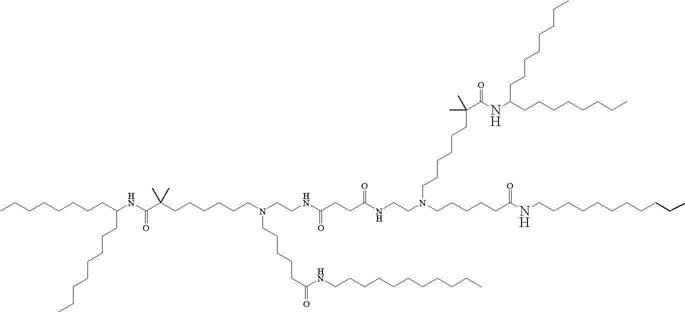 [SMILES: CCCCCCCCCCCNC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)NCCCCCCCCCCC] | |
| A86 | 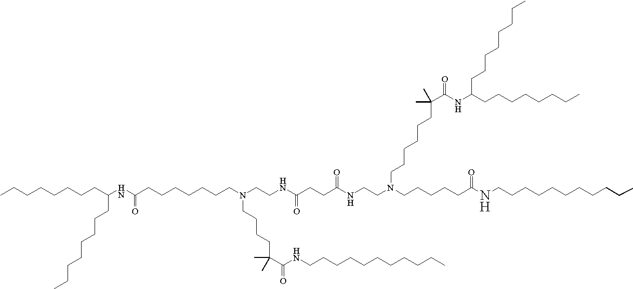 [SMILES: CCCCCCCCCCCNC(=O)CCCCCN(CCCCCCC(C)(C)C(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)NCCCCCCCCCCC] | |
| A87 | 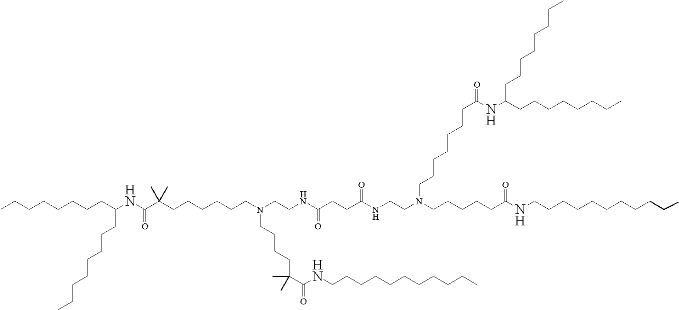 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] | |
| A88 | 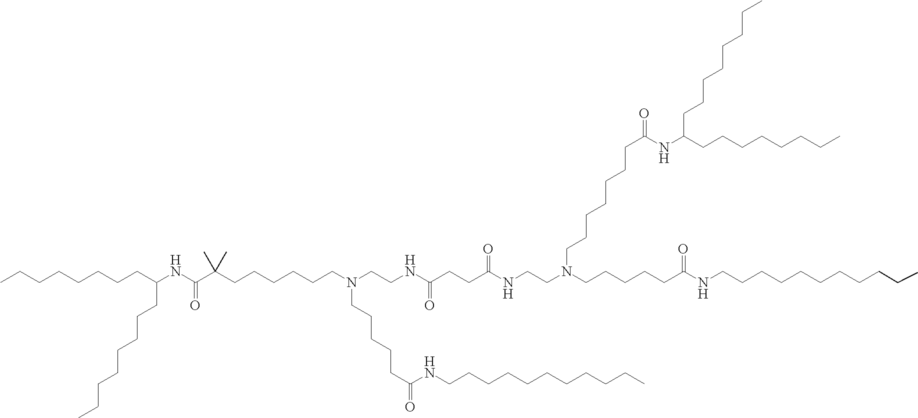 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)NCCCCCCCCCCC] | |
| A89 | 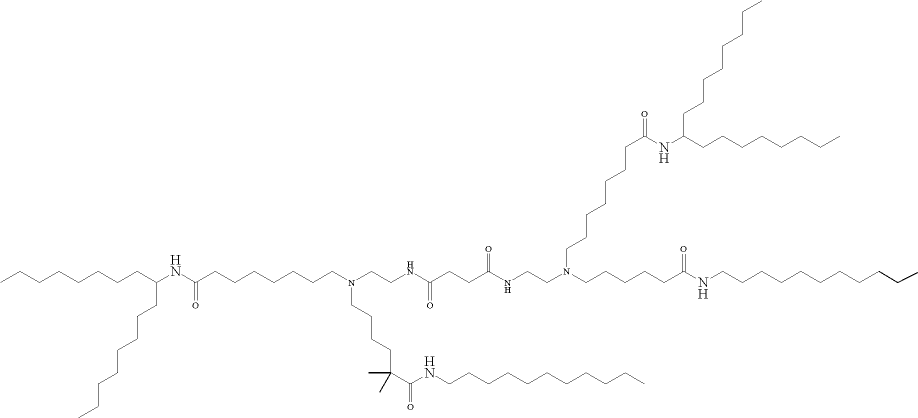 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)NCCCCCCCCCCC] | |
| A90 | 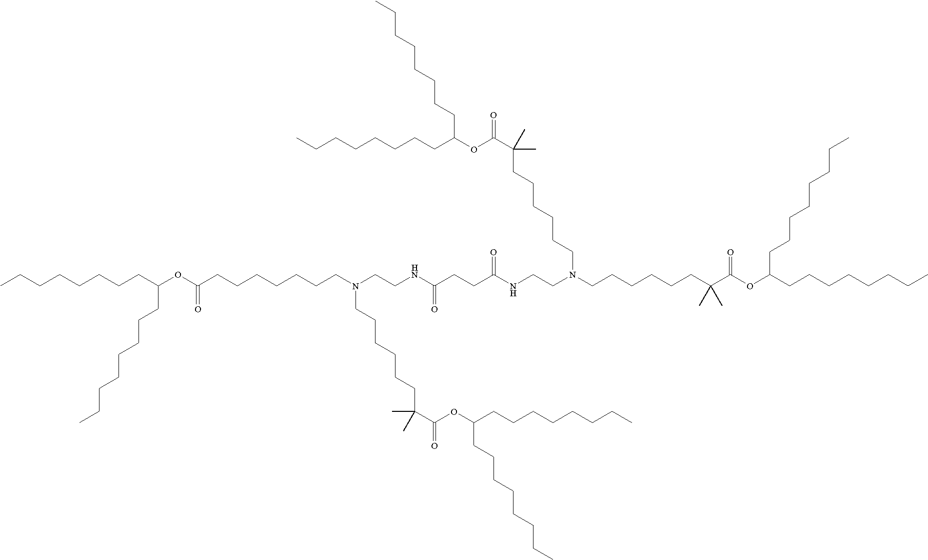 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A91 | 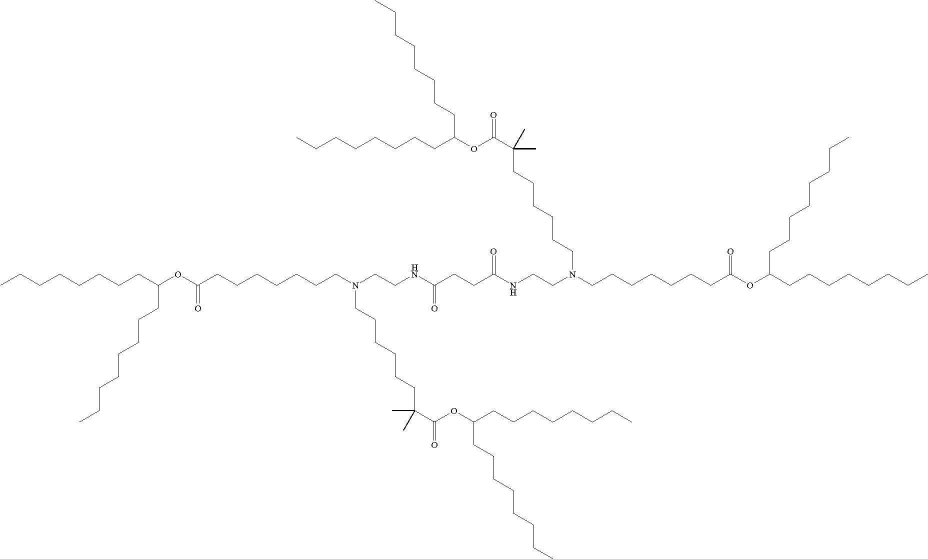 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCCCC(C)(C)C(=O)OC(CCCCCCCC)CCCCCCCC] | |
| A92 | 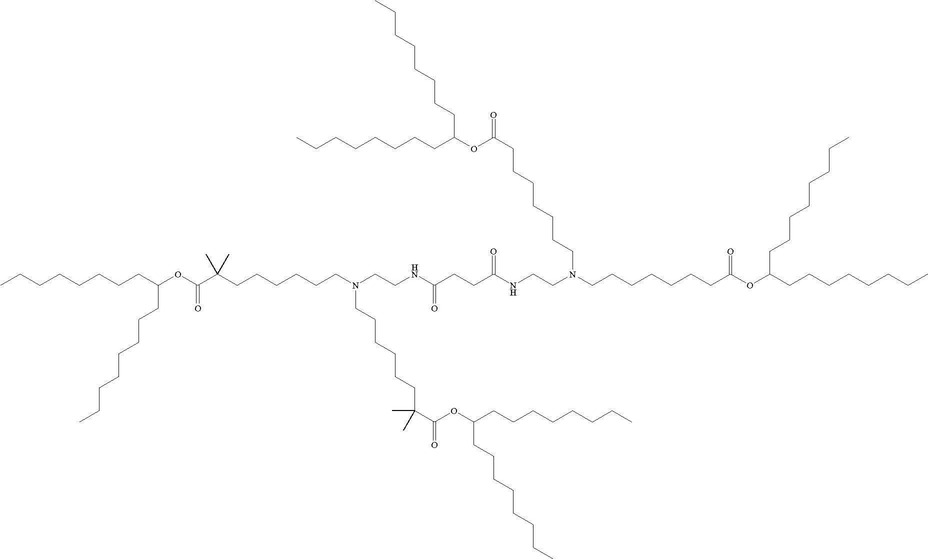 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCCC(C)(C)C(=O)OC(CCCCCCCC)CCCCCCCC] | |
| A93 | 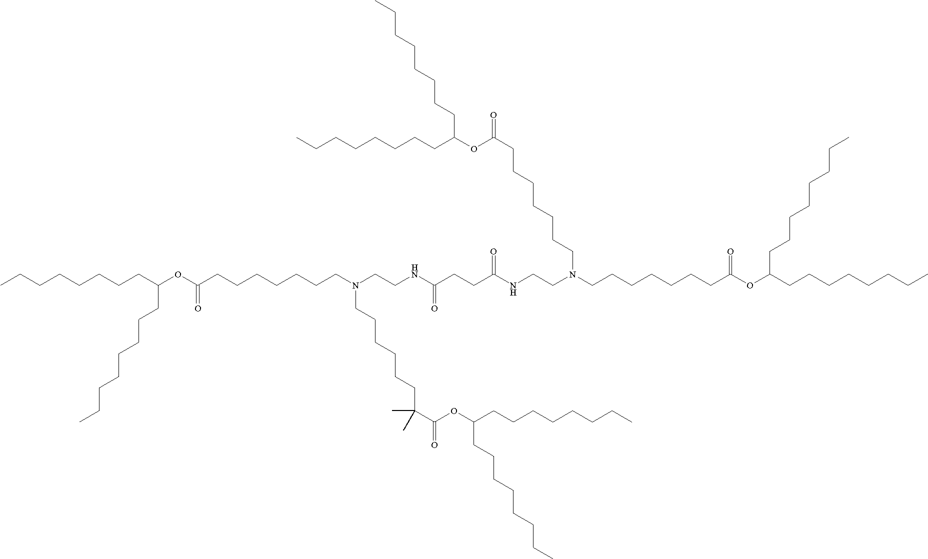 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCCC(C)(C)C(=O)OC(CCCCCCCC)CCCCCCCC] | |
| A94 | 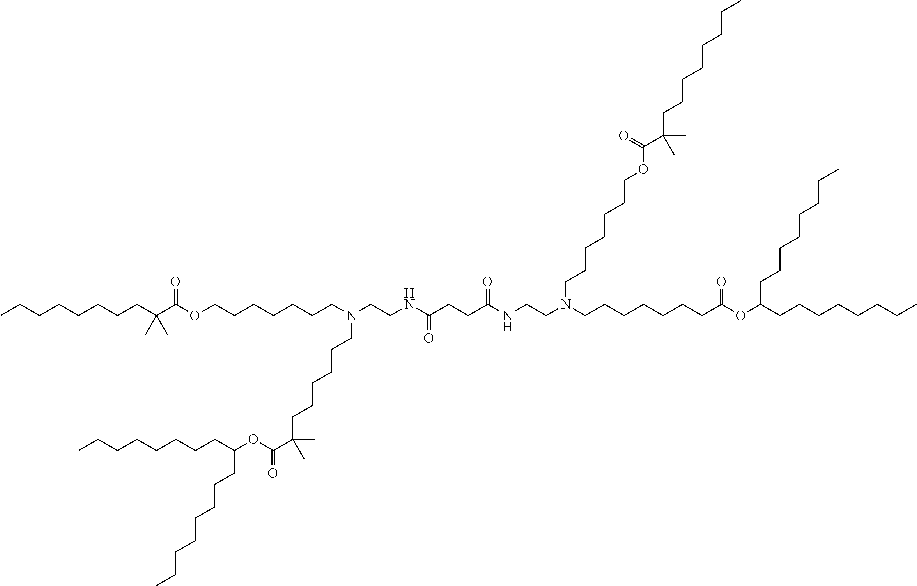 [SMILES: CCCCCCCCC(CCCCCCCC)OC(=O)CCCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] | |
| A95 | 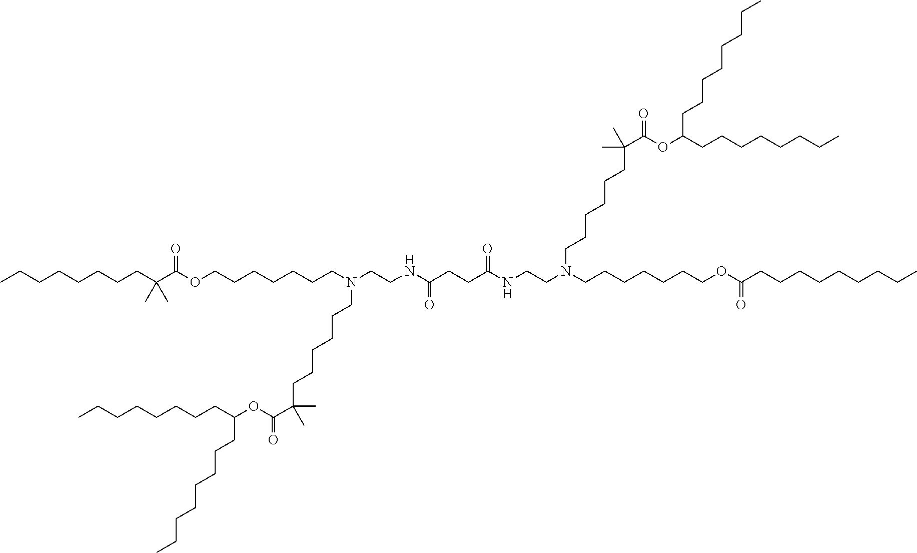 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] | |
| A96 | 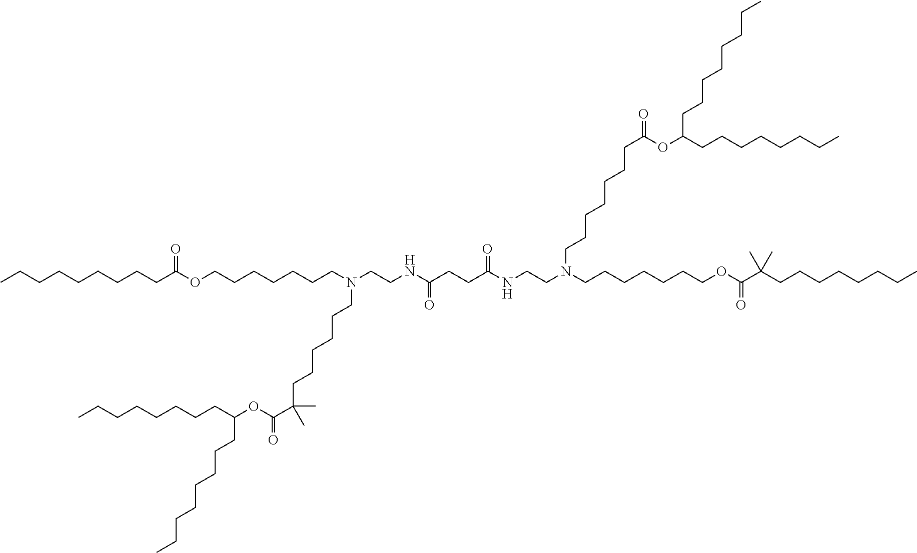 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] | |
| A97 | 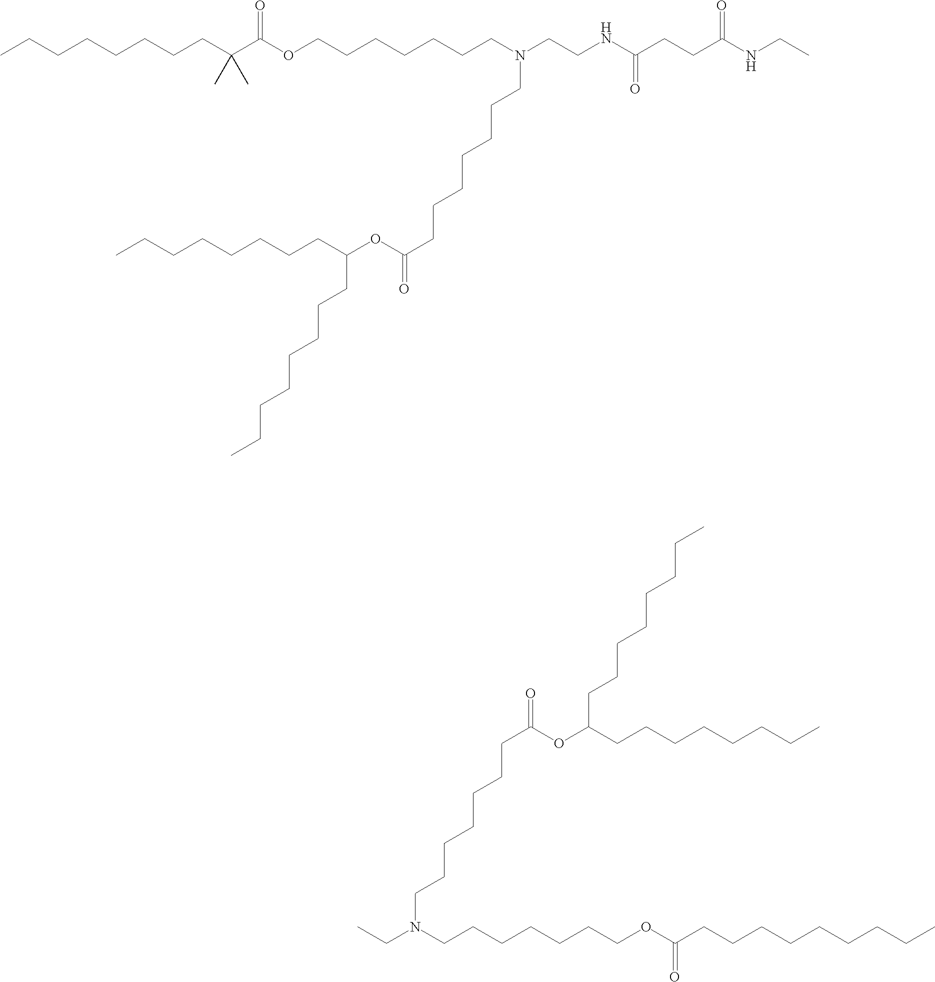 | |
| A98 | 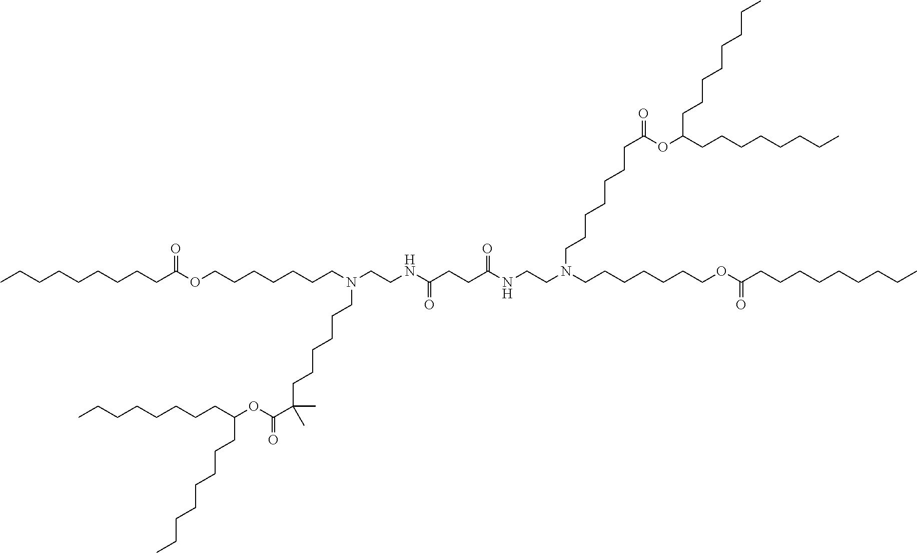 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)CCCCCCCCC)CCCCCCC(C)(C)C(=O)OC(CCCCCCCC)CCCCCCCC] | |
| A99 | 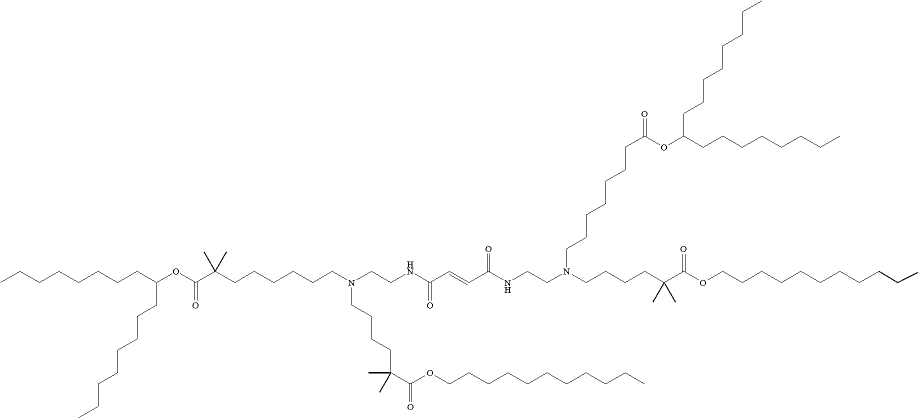 [SMILES: CCCCCCCCCCCOC(=O)C(C)(C)CCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | |
| A100 | 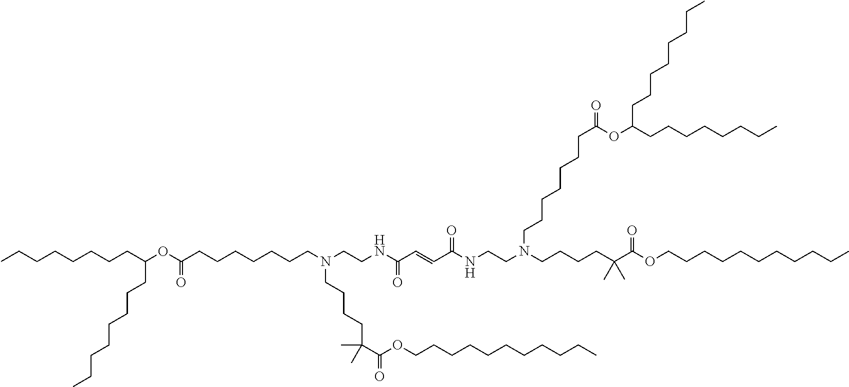 [SMILES: CCCCCCCCCCCOC(=O)C(C)(C)CCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | |
| A101 | 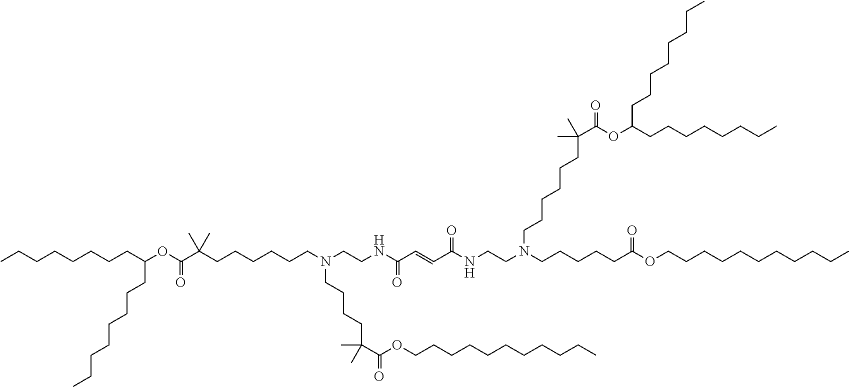 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | |
| A102 | 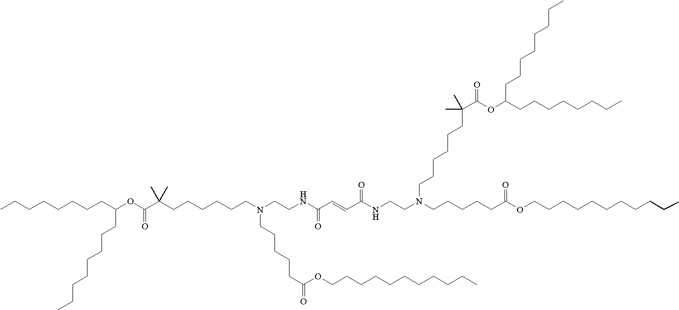 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] | |
| A103 | 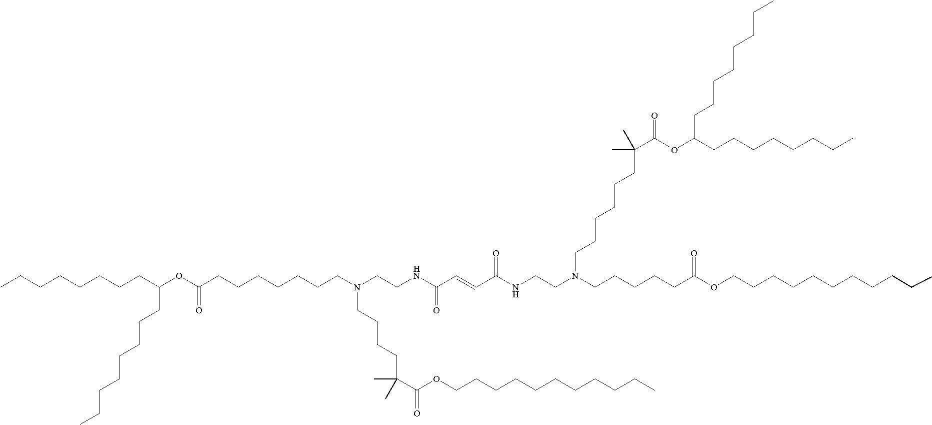 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | |
| A104 | 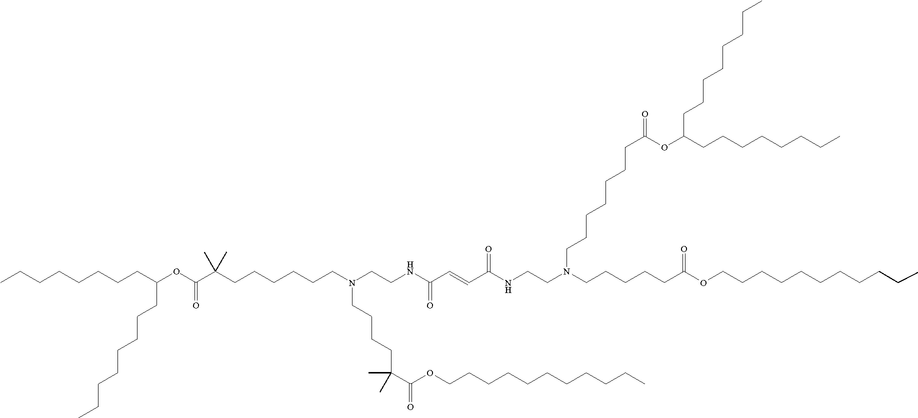 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] | |
| A105 | 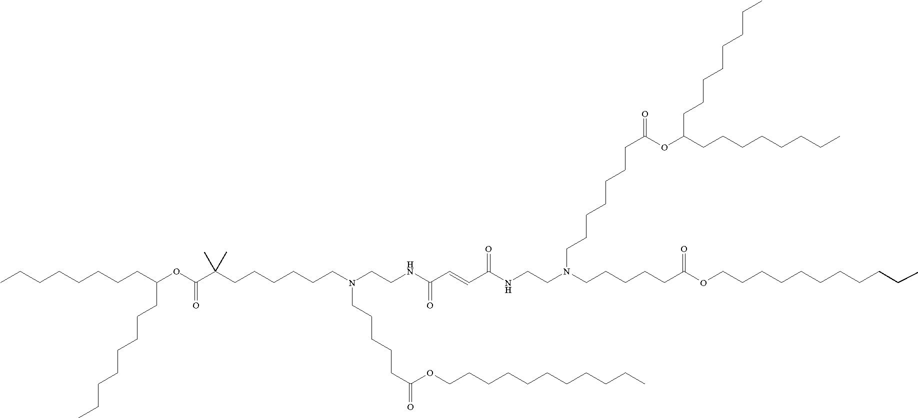 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] | |
| A106 | 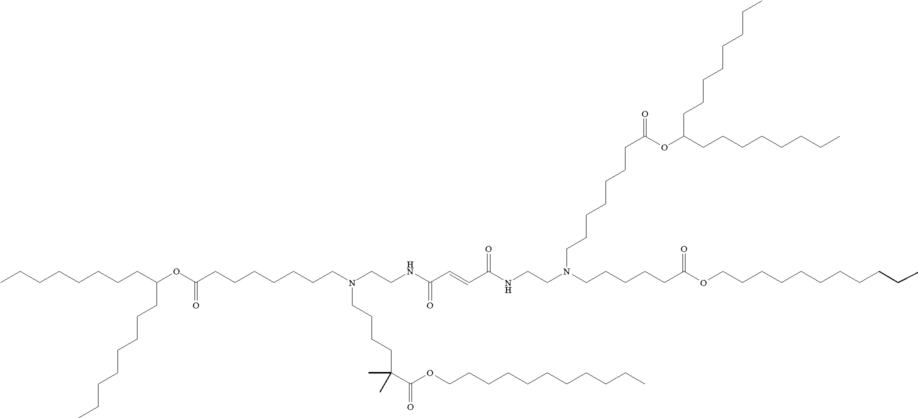 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] | |
| B1 | 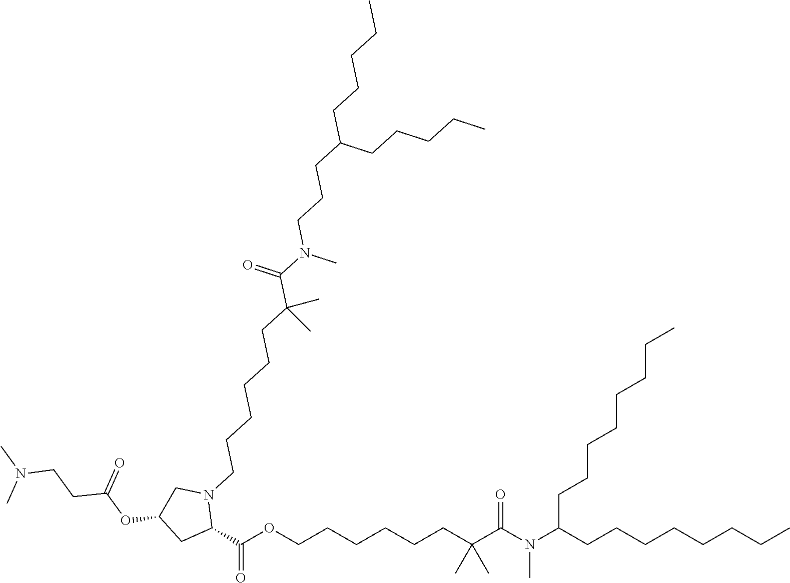 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCC(C)(C)C(=O)N(C)CCCC(CCCCC)CCCCC] | |
| B2 | 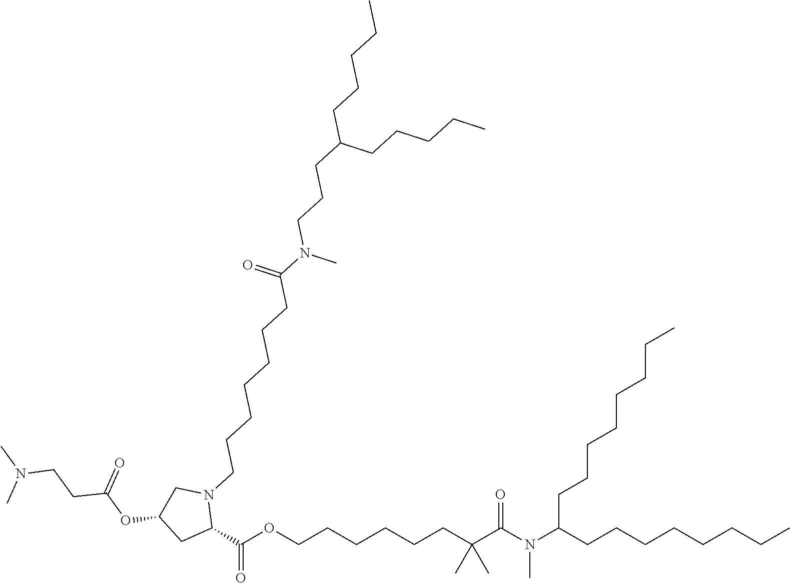 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCCC(=O)N(C)CCCC(CCCCC)CCCCC] | |
| B3 | 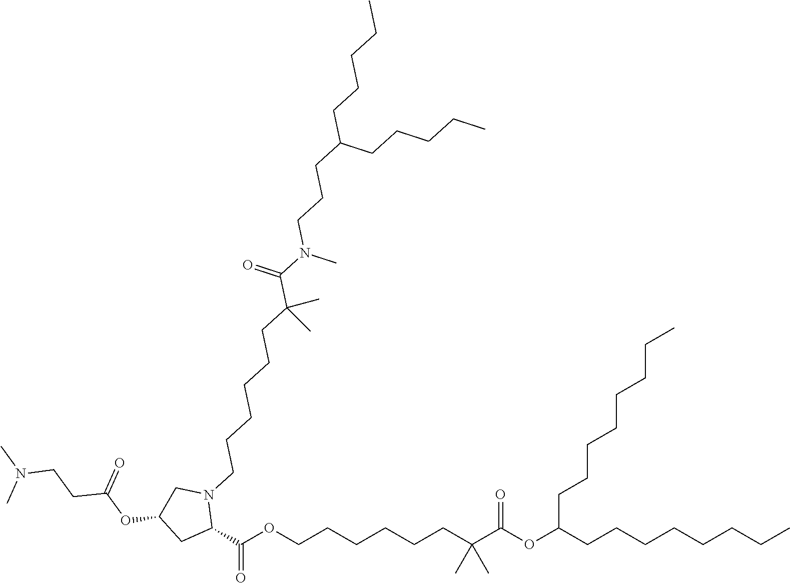 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)N(C)CCCC(CCCCC)CCCCC] | |
| B4 | 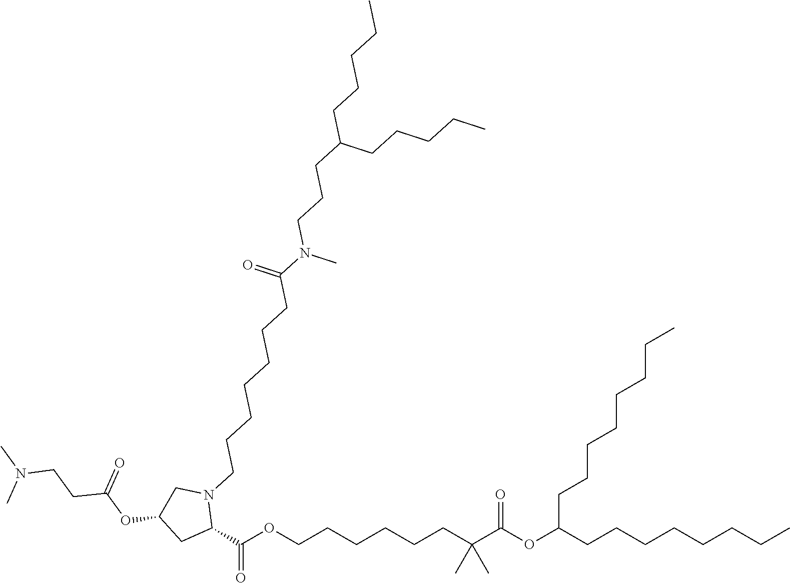 [SMILES: CCCCCCCCC(CCCCCCCC)OC(=O)C(C)(C)CCCCCCOC(=O)[C@@H]1C[C@H](OC(=O)CCN(C)C)CN1CCCCCCCC(=O)N(C)CCCC(CCCCC)CCCCC] | |
| B5 | 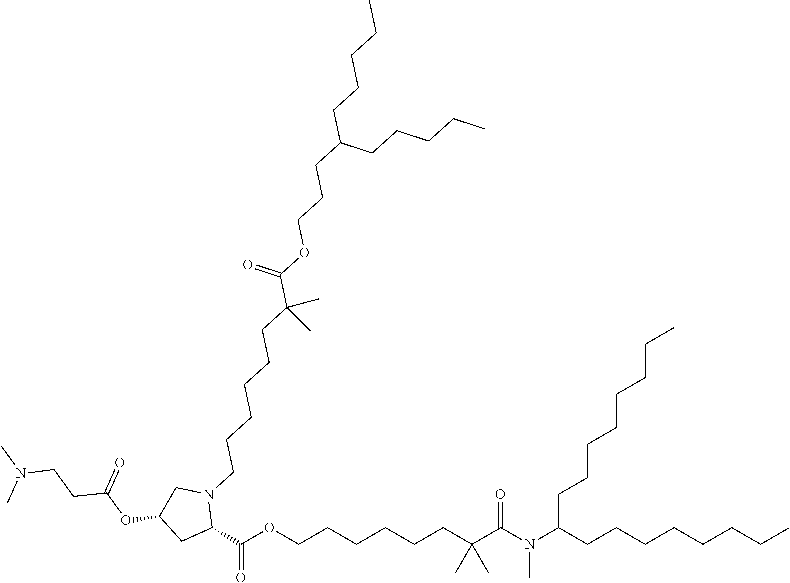 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCC(C)(C)C(=O)OCCCC(CCCCC)CCCCC] | |
| B6 | 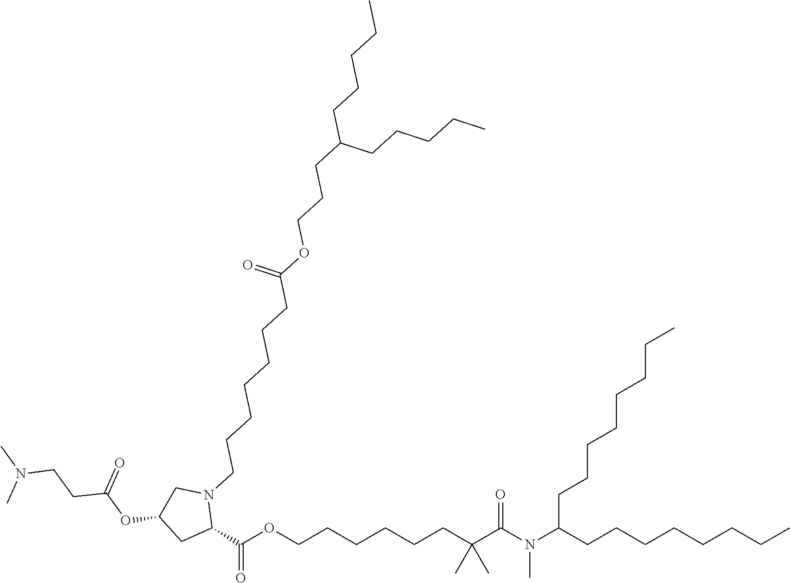 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCCC(=O)OCCCC(CCCCC)CCCCC] | |
| B7 | 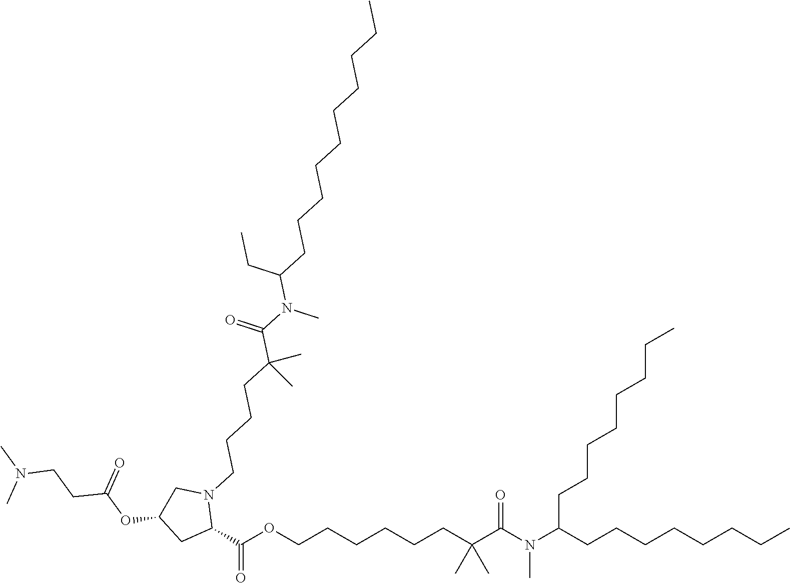 [SMILES: CCCCCCCCCCC(CC)N(C)C(=O)C(C)(C)CCCCN1C[C@@H](OC(=O)CCN(C)C)C[C@H]1C(=O)OCCCCCCC(C)(C)C(=O)N(C)C(CCCCCCCC)CCCCCCCC] | |
| B8 | 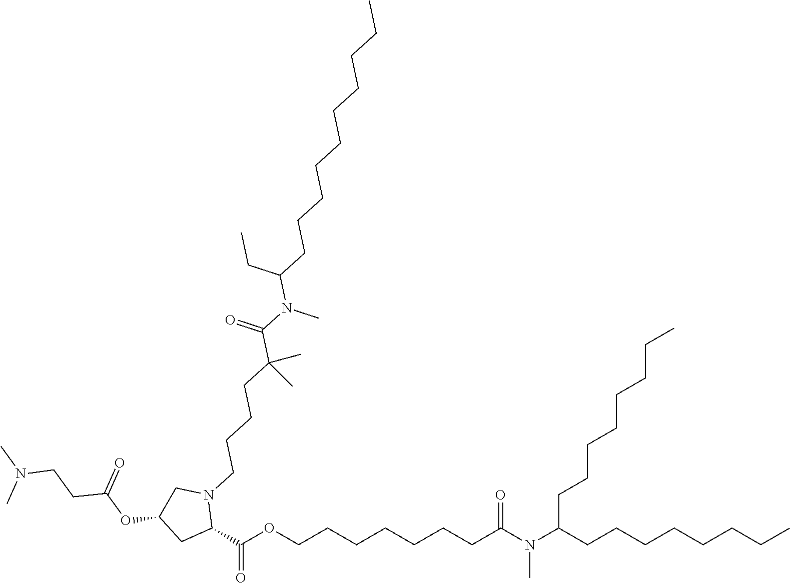 [SMILES: CCCCCCCCCCC(CC)N(C)C(=O)C(C)(C)CCCCN1C[C@@H](OC(=O)CCN(C)C)C[C@H]1C(=O)OCCCCCCCC(=O)N(C)C(CCCCCCCC)CCCCCCCC] | |
| B9 | 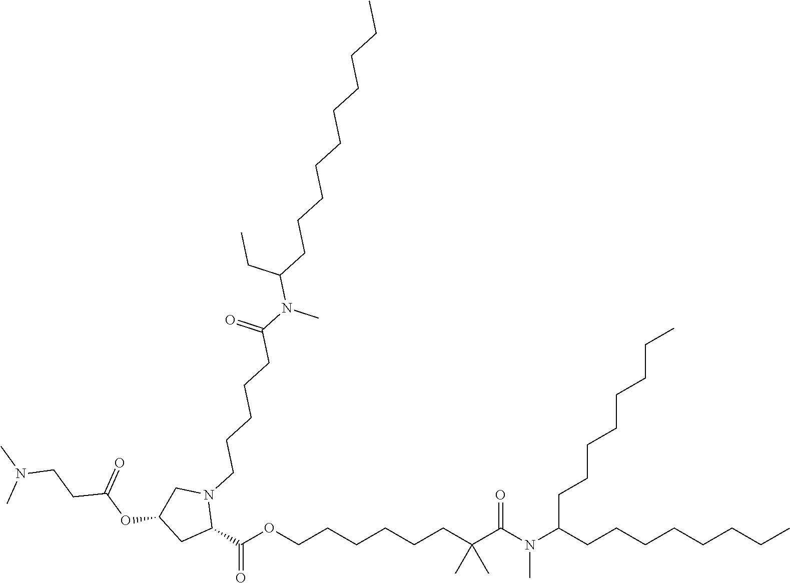 [SMILES: CCCCCCCCCCC(CC)N(C)C(=O)CCCCCN1C[C@@H](OC(=O)CCN(C)C)C[C@H]1C(=O)OCCCCCCC(C)(C)C(=O)N(C)C(CCCCCCCC)CCCCCCCC] | |
| B10 | 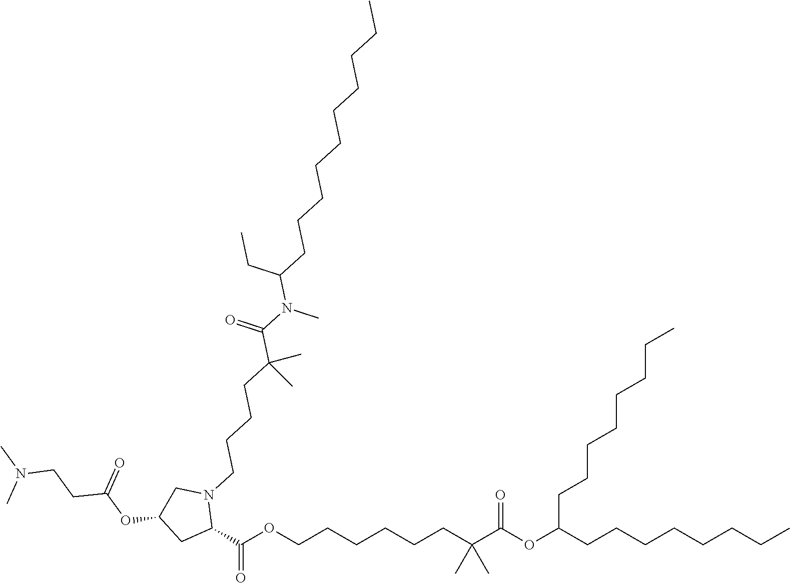 [SMILES: CCCCCCCCCCC(CC)N(C)C(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] | |
| B11 | 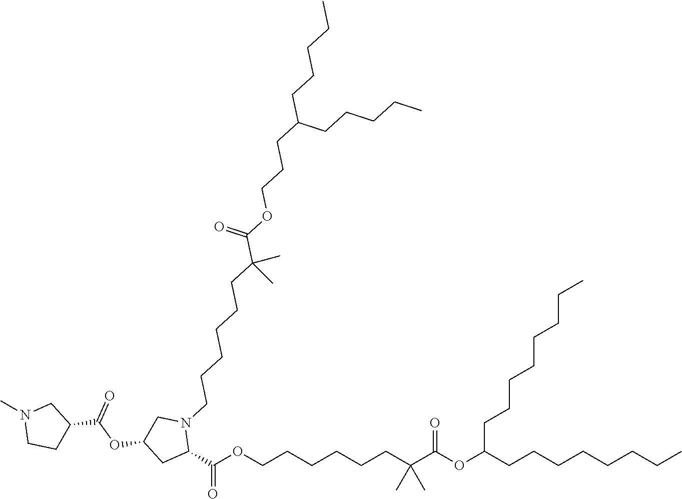 [SMILES: CCCCCCCCC(CCCCCCCC)OC(=O)C(C)(C)CCCCCCOC(=O)[C@@H]1C[C@H](OC(=O)[C@@H]2CCN(C)C2)CN1CCCCCCC(C)(C)C(=O)OCCCC(CCCCC)CCCCC] | |
| B12 | 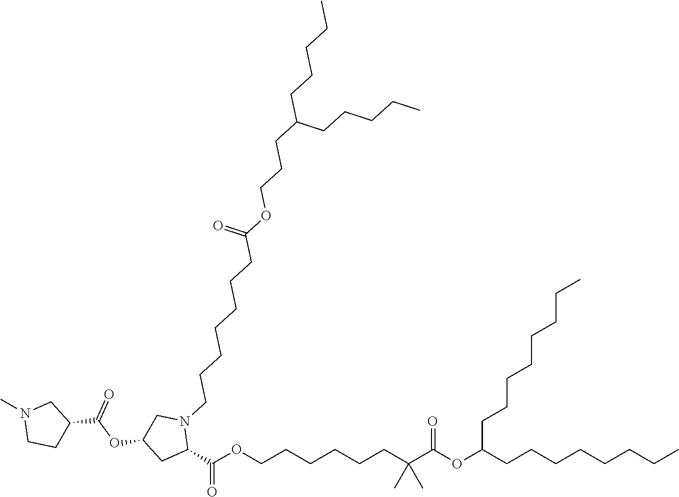 [SMILES: CCCCCCCCC(CCCCCCCC)OC(=O)C(C)(C)CCCCCCOC(=O)[C@@H]1C[C@H](OC(=O)[C@@H]2CCN(C)C2)CN1CCCCCCCC(=O)OCCCC(CCCCC)CCCCC] | |
| B13 | 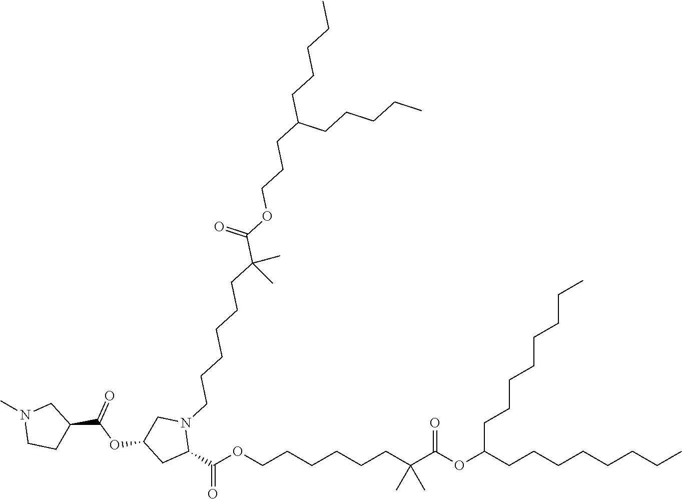 [SMILES: CCCCCCCCC(CCCCCCCC)OC(=O)C(C)(C)CCCCCCOC(=O)[C@@H]1C[C@H](OC(=O)[C@H]2CCN(C)C2)CN1CCCCCCC(C)(C)C(=O)OCCCC(CCCCC)CCCCC] | |
| B14 | 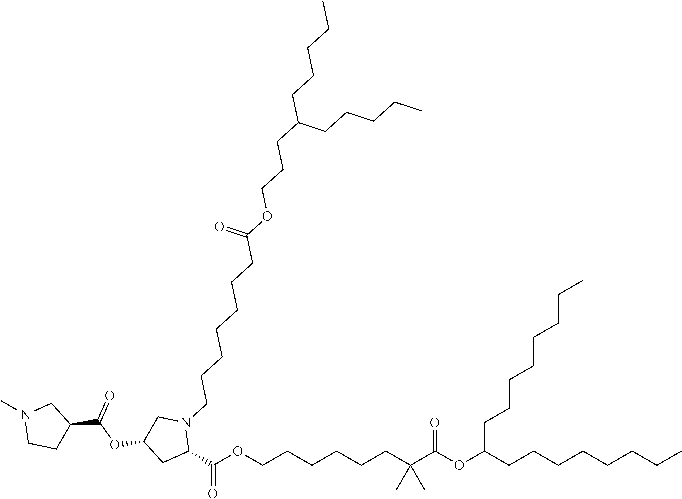 [SMILES: CCCCCCCCC(CCCCCCCC)OC(=O)C(C)(C)CCCCCCOC(=O)[C@@H]1C[C@H](OC(=O)[C@H]2CCN(C)C2)CN1CCCCCCCC(=O)OCCCC(CCCCC)CCCCC] | |
| B15 | 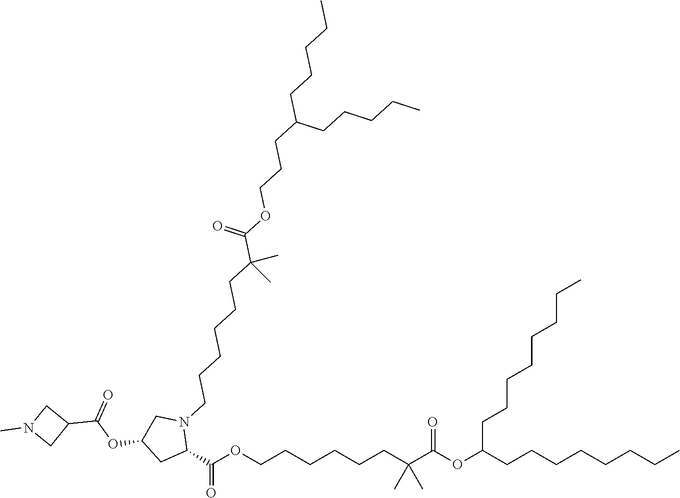 [SMILES: CCCCCCCCC(CCCCCCCC)OC(=O)C(C)(C)CCCCCCOC(=O)[C@@H]1C[C@H](OC(=O)C2CN(C)C2)CN1CCCCCCC(C)(C)C(=O)OCCCC(CCCCC)CCCCC] | |
| B16 | 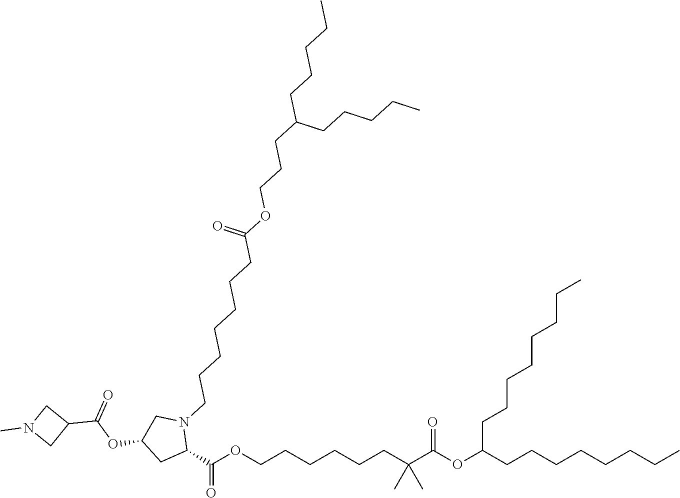 [SMILES: CCCCCCCCC(CCCCCCCC)OC(=O)C(C)(C)CCCCCCOC(=O)[C@@H]1C[C@H](OC(=O)C2CN(C)C2)CN1CCCCCCCC(=O)OCCCC(CCCCC)CCCCC] | |
| B17 | 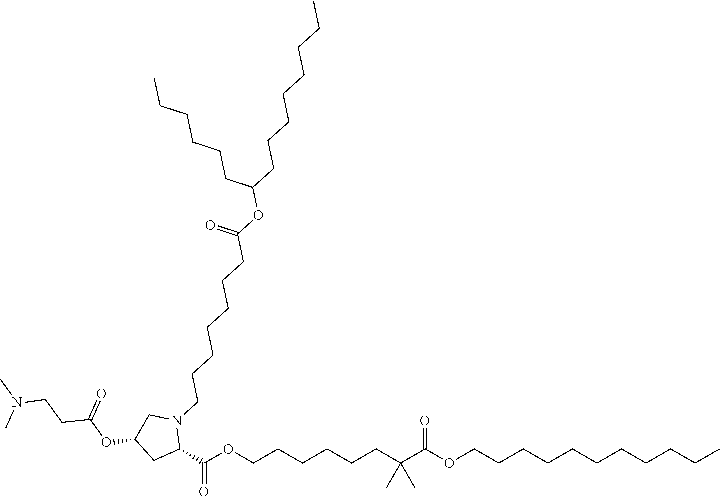 [SMILES: CCCCCCCCCCCOC(=O)C(C)(C)CCCCCCOC(=O)[C@@H]1C[C@H](OC(=O)CCN(C)C)CN1CCCCCCCC(=O)OC(CCCCCC)CCCCCCCC] | |
| B18 | 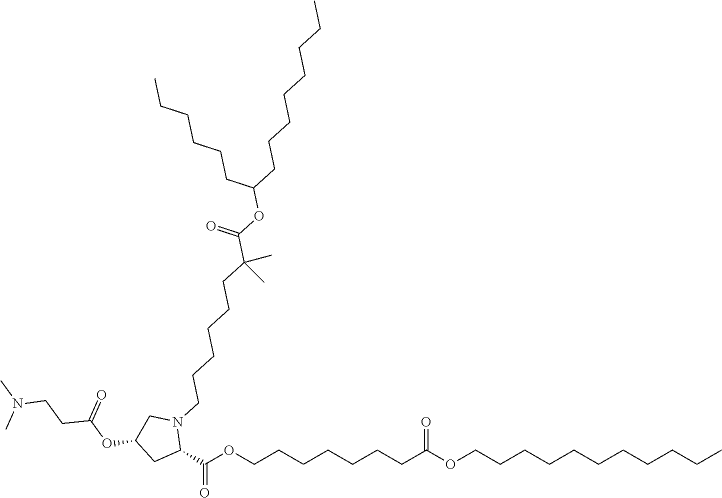 [SMILES: CCCCCCCCCCCOC(=O)CCCCCCCOC(=O)[C@@H]1C[C@H](OC(=O)CCN(C)C)CN1CCCCCCC(C)(C)C(=O)OC(CCCCCC)CCCCCCCC] | |
| B19 | 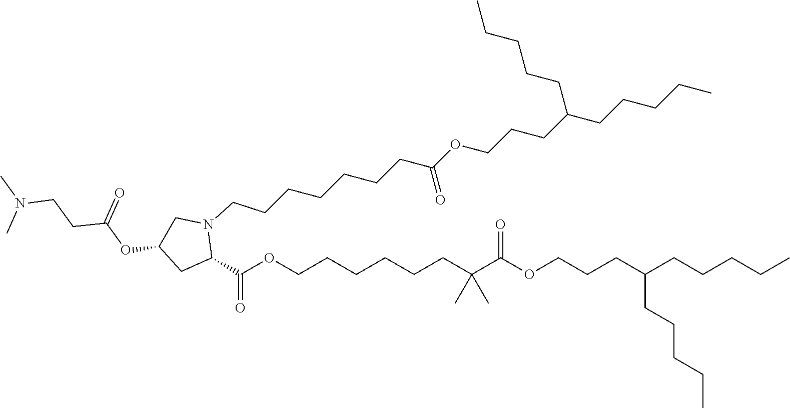 [SMILES: CCCCCC(CCCCC)CCCOC(=O)CCCCCCCN1C[C@@H](OC(=O)CCN(C)C)C[C@H]1C(=O)OCCCCCCC(C)(C)C(=O)OCCCC(CCCCC)CCCCC] | |
| B20 | 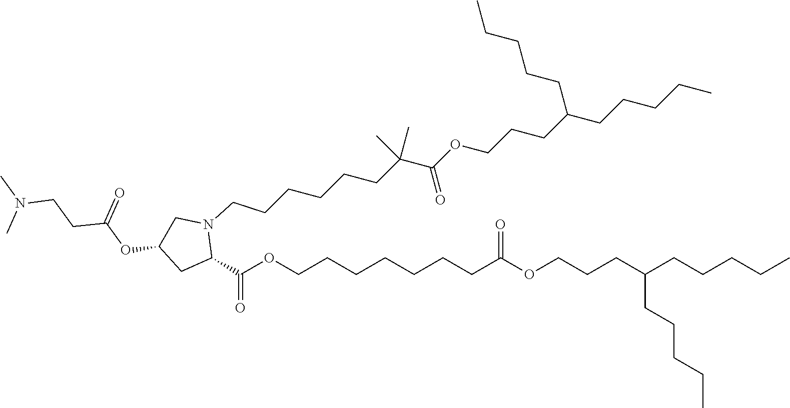 [SMILES: CCCCCC(CCCCC)CCCOC(=O)CCCCCCCOC(=O)[C@@H]1C[C@H](OC(=O)CCN(C)C)CN1CCCCCCC(C)(C)C(=O)OCCCC(CCCCC)CCCCC] | |
| B21 | 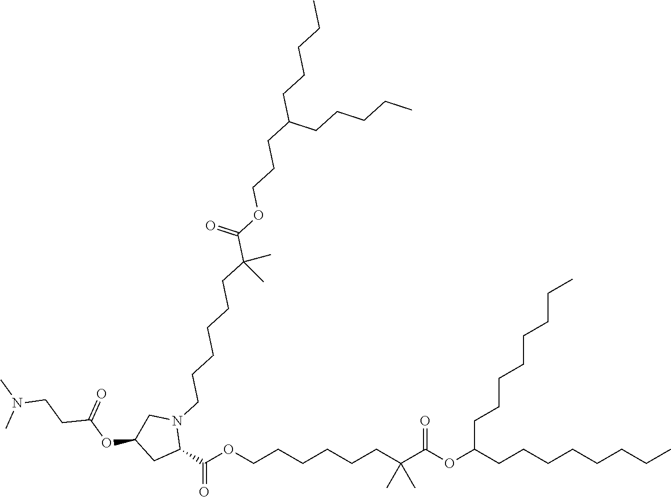 [SMILES: CCCCCCCCC(CCCCCCCC)OC(=O)C(C)(C)CCCCCCOC(=O)[C@@H]1C[C@@H](OC(=O)CCN(C)C)CN1CCCCCCC(C)(C)C(=O)OCCCC(CCCCC)CCCCC] | |
| B22 | 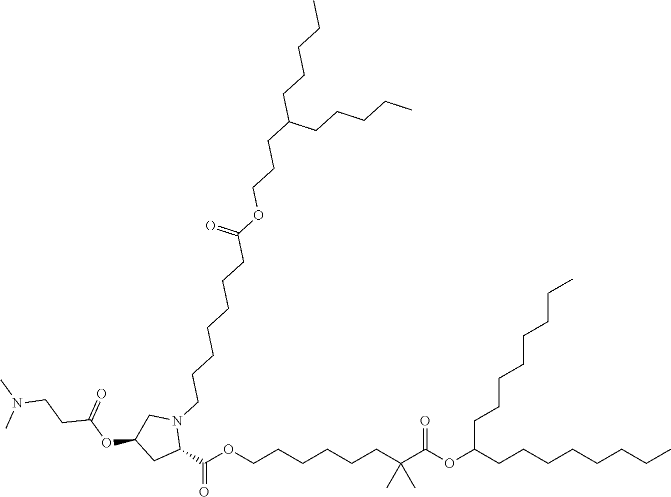 [SMILES: CCCCCCCCC(CCCCCCCC)OC(=O)C(C)(C)CCCCCCOC(=O)[C@@H]1C[C@@H](OC(=O)CCN(C)C)CN1CCCCCCCC(=O)OCCCC(CCCCC)CCCCC] | |
| B23 | 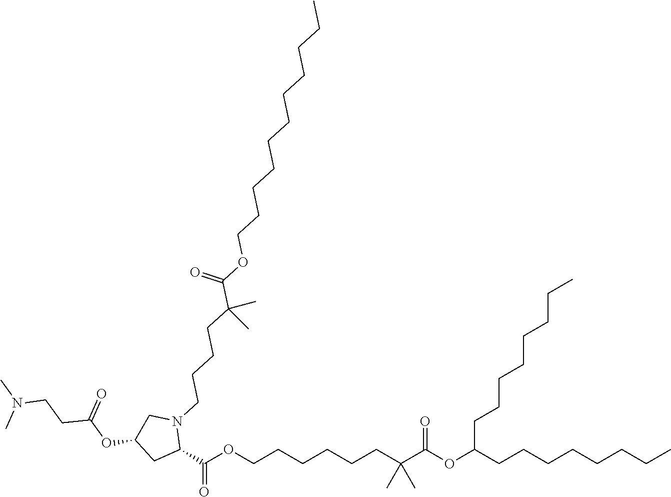 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] | |
| B24 | 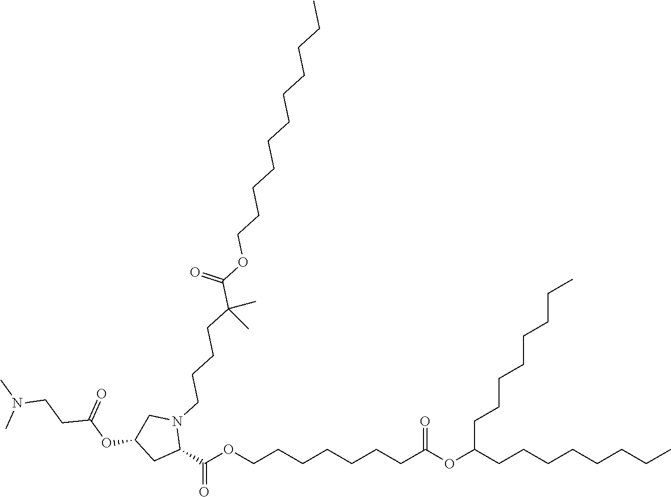 [SMILES: CCCCCCCCCCCOC(=O)C(C)(C)CCCCN1C[C@@H](OC(=O)CCN(C)C)C[C@H]1C(=O)OCCCCCCCC(=O)OC(CCCCCCCC)CCCCCCCC] | |
| B25 | 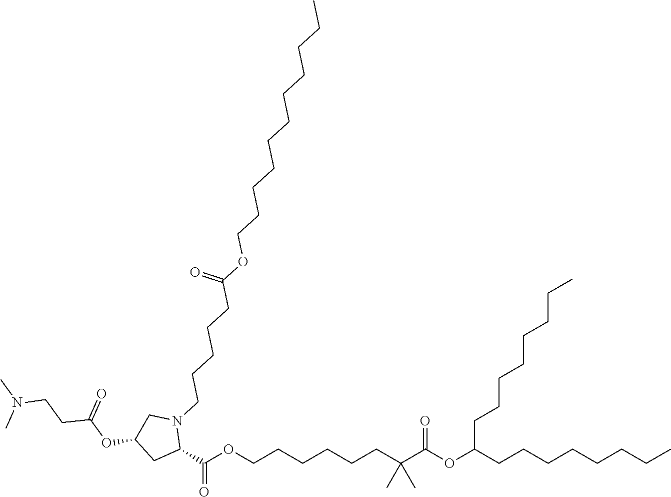 [SMILES: CCCCCCCCCCCOC(=O)CCCCCN1C[C@@H](OC(=O)CCN(C)C)C[C@H]1C(=O)OCCCCCCC(C)(C)C(=O)OC(CCCCCCCC)CCCCCCCC] | |

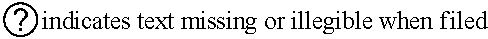

### Process of Preparing Exemplary Lipid Compounds

Disclosed herein are also various processes to make the exemplary lipid compounds.

In some embodiments, provided herein is a process for making a lipid comprising at least one head group and at least one tail group of formula (TI) or (TI′)

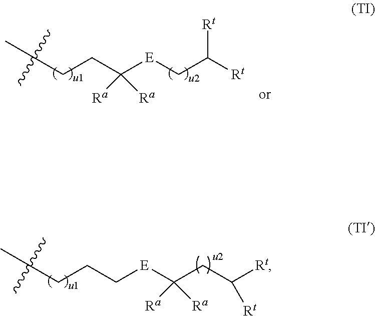

wherein:
E is each independently a biodegradable group;
R^{a} is each independently C₁-C₅ alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
[symbol] represents the bond connecting the tail group to the head group; and
wherein the lipid has a pKa from about 4 to about 8.

The process comprises
reacting a first precursor compound of the tail group of formula (TI) or (TI′)

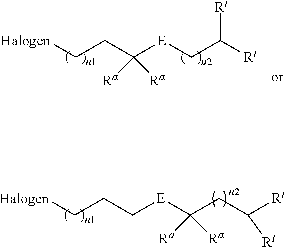

with a precursor compound of the head group, wherein the precursor compound of the head group comprises one or more attaching points for the tail group(s), each attaching point containing a functional group reactive to halogen, thereby forming a lipid by attaching at least one tail group of formula (TI) or (TI′) to the head group at the one or more attaching points.

In some embodiments, one or more attaching points for the tail group in the precursor compound of the head group contains one or more N.

In some embodiments, one or more attaching points for the tail group in the precursor compound of the head group further comprise a non-N functional group, and the one or more N contained at the one or more attaching points of the precursor compound of the head group is protected so that the attaching points containing the non-N functional group is reacted with the precursor compound of the tail group. The process then further comprises:
deprotecting the one or more N contained at the one or more attaching points of the head group of the lipid; and
reacting a second precursor compound of the tail group of formula (TI) or (TI′)

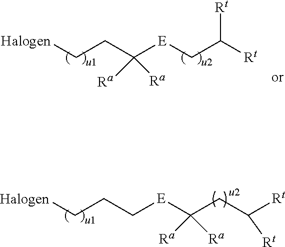

with the lipid containing the one or more deprotected N at the one or more attaching points of the head group, thereby forming a lipid by attaching a second tail group of formula (TI) or (TI′) to the head group at the one or more attaching points. In some embodiments, the second precursor compound of the tail group is the same as the first precursor compound of the tail group. Thus, the final lipid contains multiple tail groups that are the same. In some embodiments, the second precursor compound of the tail group is different than the first precursor compound of the tail group. Thus, the final lipid contains multiple different tail groups.

In some embodiments, at least one tail group has one of the following formulas:

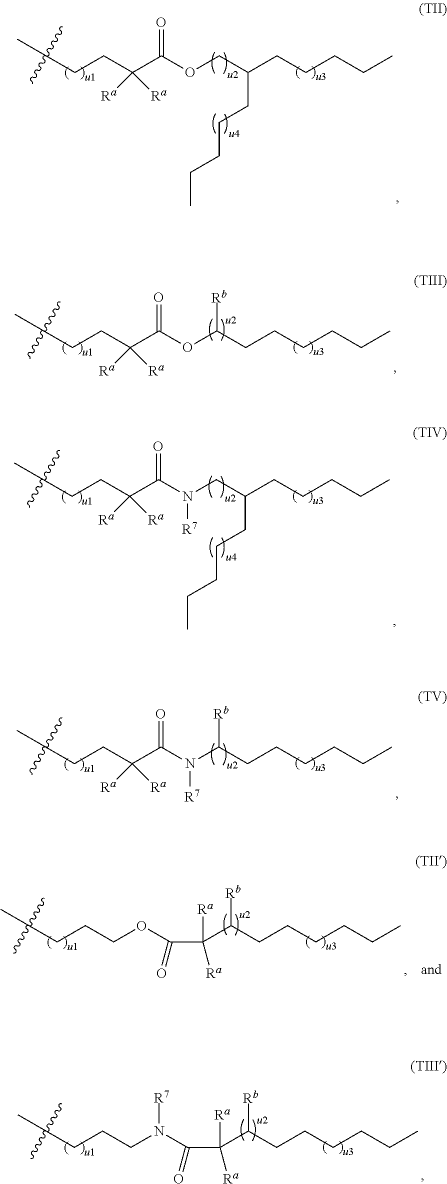

R⁷ is each independently H or methyl;
R^{b} is in each occasion independently H or C₁-C₄ alkyl; and
u3 and u4 are each independently 0, 1, 2, 3, 4, 5, 6, or 7.

In some embodiments, each R^{a} in the above formulas is methyl.

In some embodiments, provided herein is a process for making a lipid comprising at least one head group and at least one tail group, having the formula of

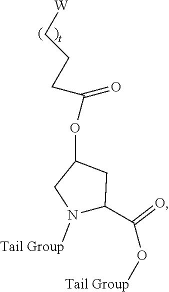

wherein:
   Tail Group is

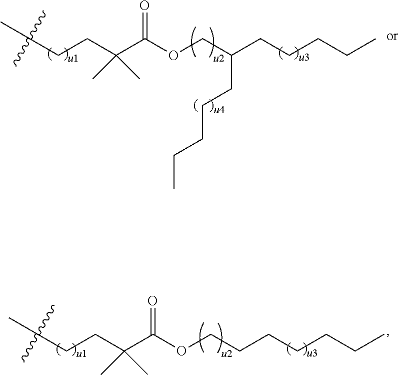

u1 and u2 are each independently 0, 1, 2, 3, 4, 5, 6, or 7,
u3 and u4 are each independently 0, 1, 2, 3, or 4.
W is hydroxyl, hydroxyalkyl, or one of the following moieties:

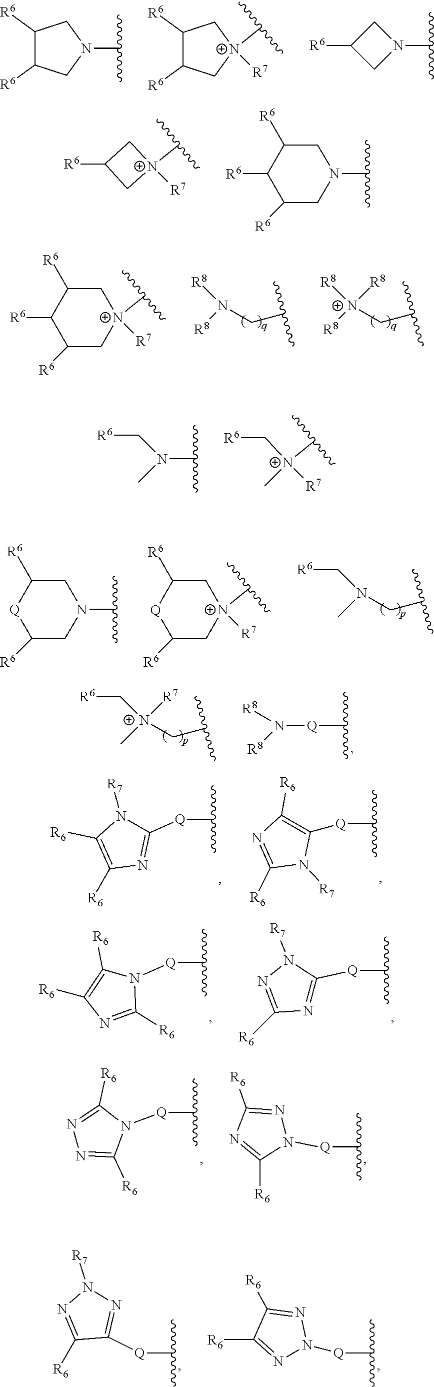

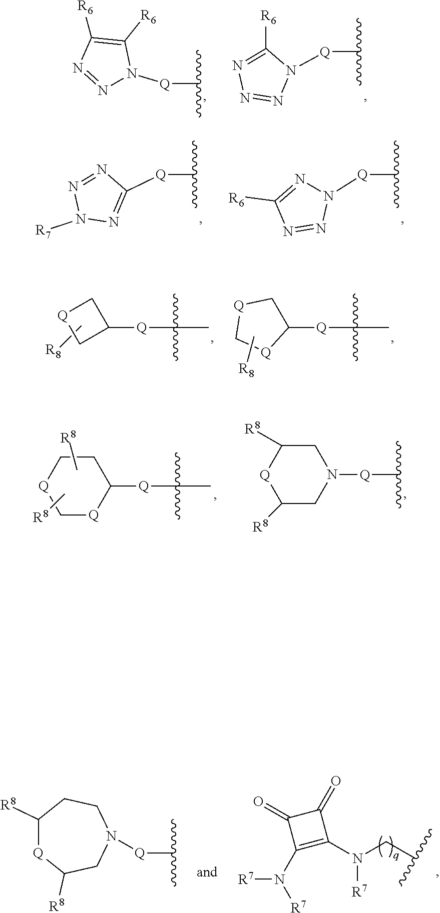

wherein:
   each Q is independently absent, —O—, —C(O)—, —C(S)—, —C(O)O—, —(CH₂)_{q}—C(R⁷)₂, —C(O)N(R⁷)—, —C(S)N(R⁷)—, or —N(R⁷);
   R⁶ is independently H, alkyl, hydroxyl, hydroxyalkyl, alkoxy, —O-alkylene-O-alkyl, —O-alkylene-N(R⁷)₂, amino, alkylamino, aminoalkyl, thiol, thiolalkyl, or N⁺(R⁷)₃-alkylene-Q-;
   each R⁸ is independently H, alkyl, hydroxyalkyl, amino, aminoalkyl, alkylamino, thiol, thiolalkyl, heterocyclyl, heteroaryl; or two R⁸ together with the nitrogen atom form a ring, optionally substituted with one or more alkyl, hydroxy, hydroxyalkyl, alkoxy, alkylaminoalkyl, alkylamino, or aminoalkyl;
   q is 0, 1, 2, 3, 4, or 5; and
   p is 0, 1, 2, 3, 4, or 5.

The process comprises
reacting compound

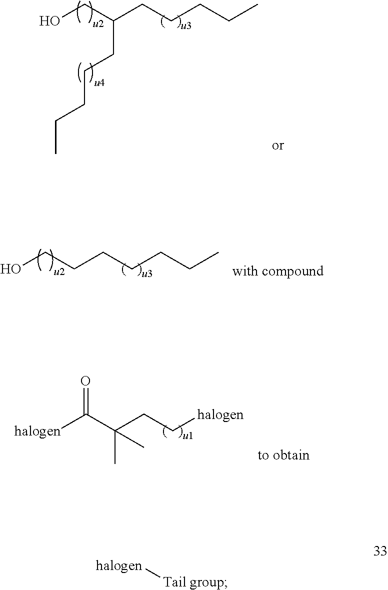

reacting compound 33 with compound

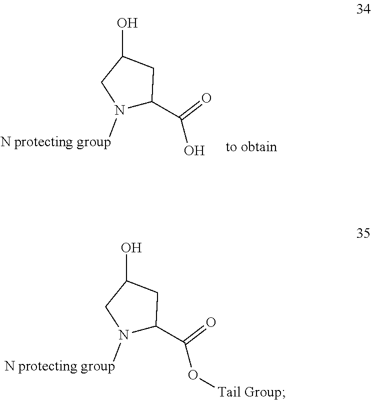

removing N protecting group of compound 35 to obtain compound

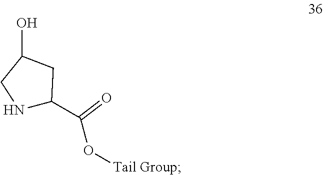

reacting compound 36 with compound [symbol] that may be the same or different than compound 33 to obtain compound

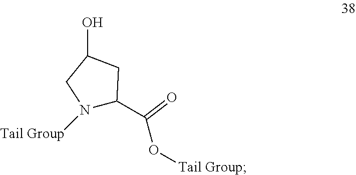

and
reacting compound 38 with compound

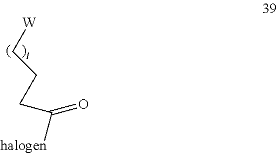

to obtain the lipid of

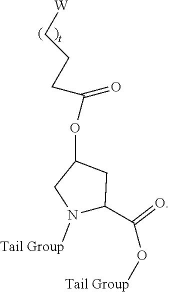

The above process can be shown in the general reaction scheme below:

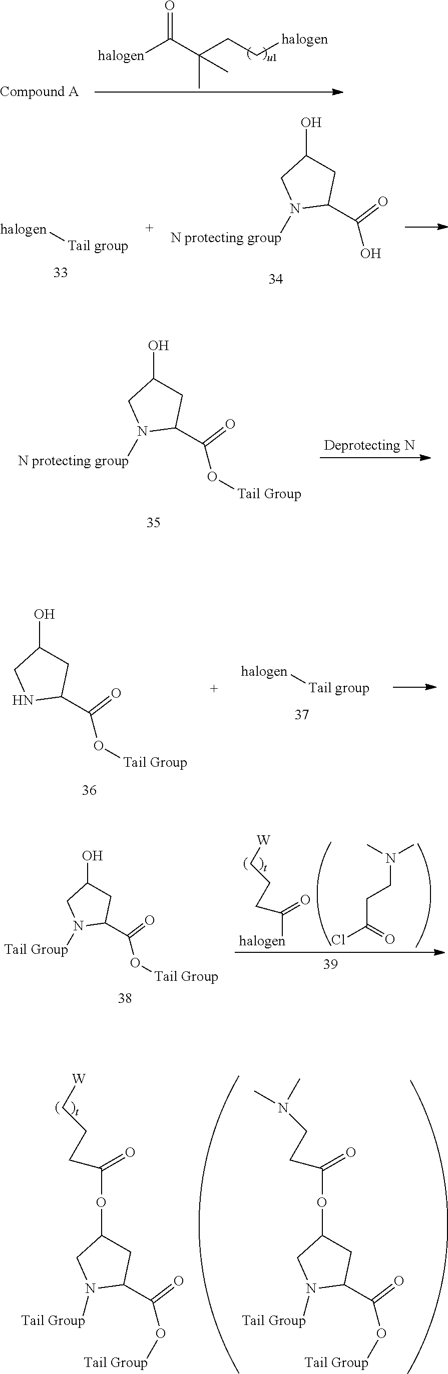

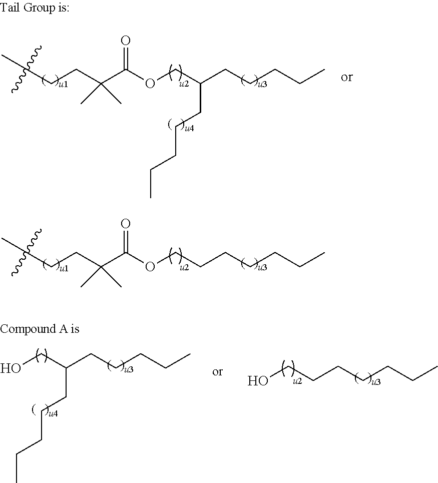

In some embodiments, provided herein is a process for making a lipid comprising at least one head group and at least one tail group, having the formula of

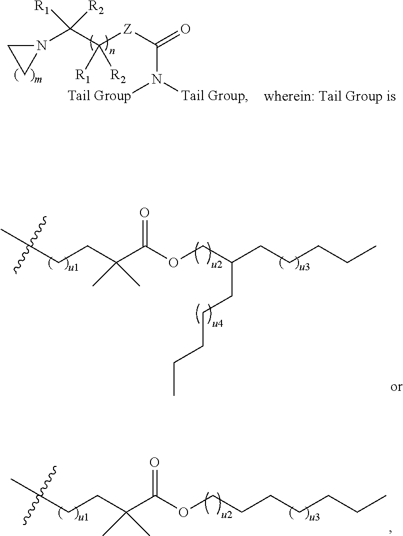

u1 and u2 are each independently 0, 1, 2, 3, 4, 5, 6, or 7,
u3 and u4 are each independently 0, 1, 2, 3, or 4.
each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ are taken together to form a cyclic ring;
each of R₁₀ and R₁₁ is independently H, C₁-C₃ branched or unbranched alkyl, C₂-C₃ branched or unbranched alkenyl; or R₁₀ and R₁₁ are taken together to form a heterocyclic ring;
m is 1, 2, 3, 4, 5, 6, 7 or 8;
n is 0, 1, 2, 3 or 4;
Z is absent, O, S, or NR₁₂, wherein R₁₂ is H or C₁-C₇ branched or unbranched alkyl;

provided that when Z is not absent, the adjacent R₁ and R₂ cannot be OH, NR₁₀R₁₁, SH.

The process comprises:
reacting compound

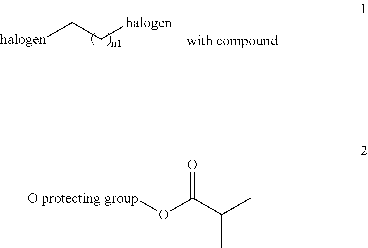

to obtain compound

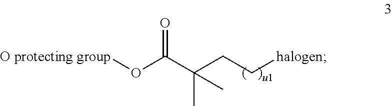

reacting compound 3 with a compound N protecting group —NH₂ to obtain compound

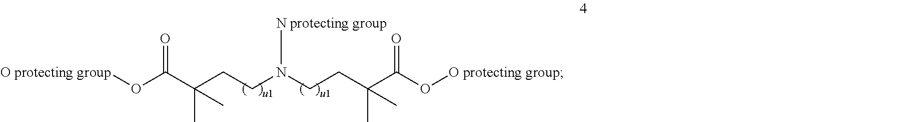

removing O protecting group of compound 4 to obtain compound

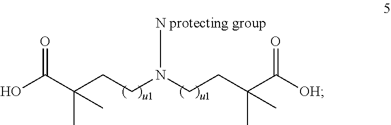

reacting compound 5 with (C(O)-Halogen)₂ to obtain compound

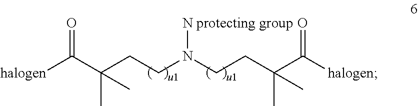

reacting compound 6 with

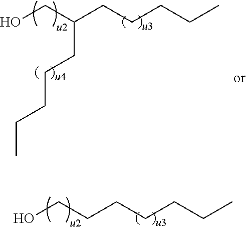

to obtain compound

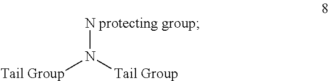

removing N protecting group of compound 8 to obtain compound

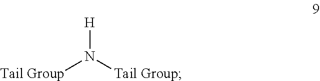

and
reacting compound 9 with compound

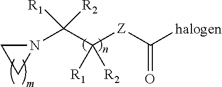

to obtain the lipid of

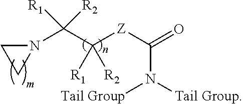

The above process can be shown in the general reaction scheme below:

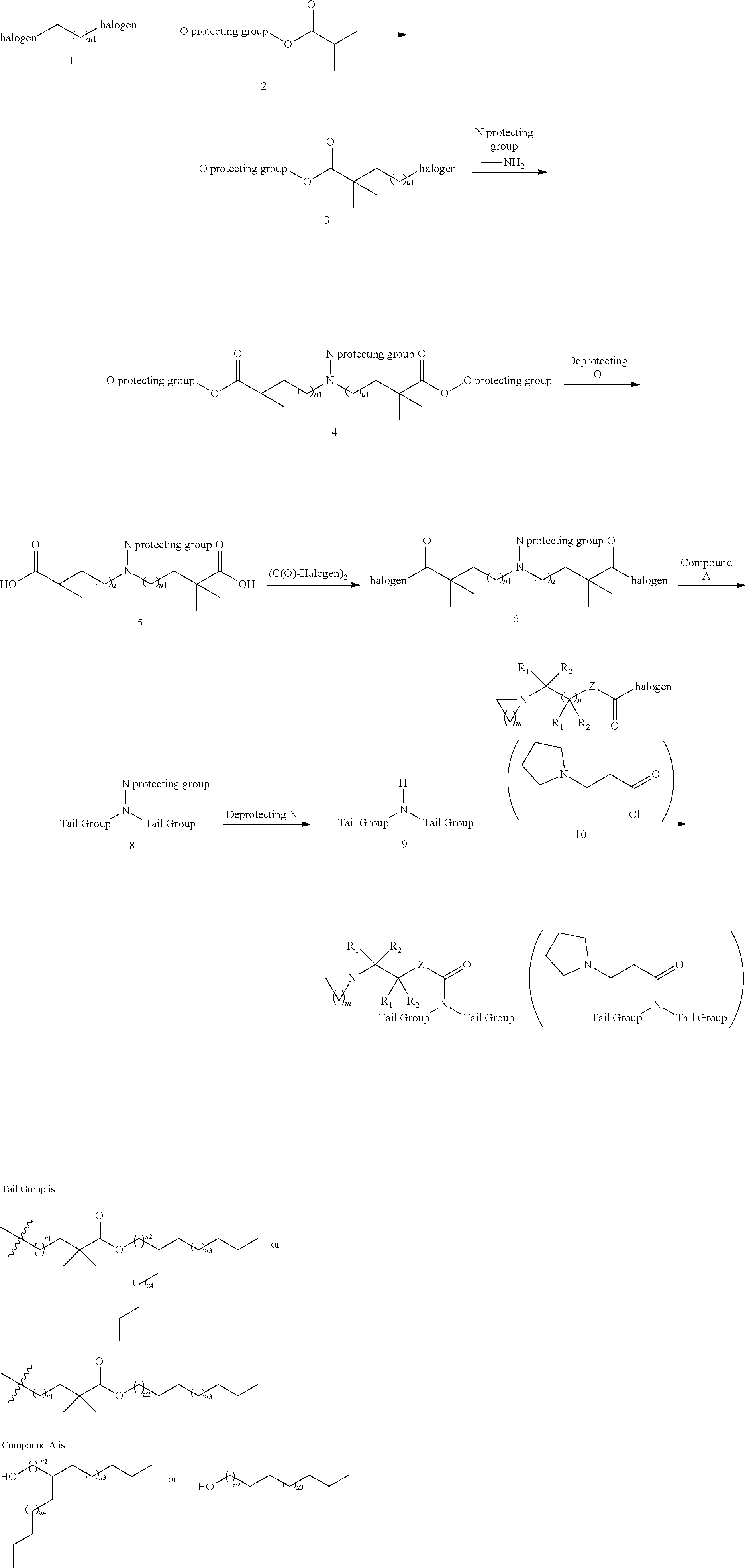

In some embodiments, provided herein is a process for making a lipid comprising at least one head group and at least one tail group, having the formula of

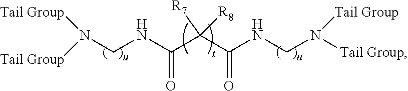

wherein:
Tail Group is

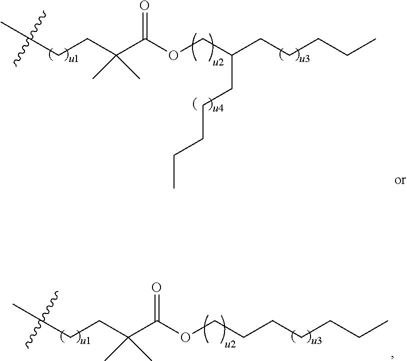

u1 and u2 are each independently 0, 1, 2, 3, 4, 5, 6, or 7,
u3 and u4 are each independently 0, 1, 2, 3, or 4.
each R₇ and R₈ are independently H, C₁-C₃ branched or unbranched alkyl, C₂-C₃ branched or unbranched alkenyl, halogen, OH, SH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; R₇ and R₈ are taken together to form a ring;
each of s, u, and t is independently 1, 2, 3, 4, or 5.

The process comprises:
reacting compound

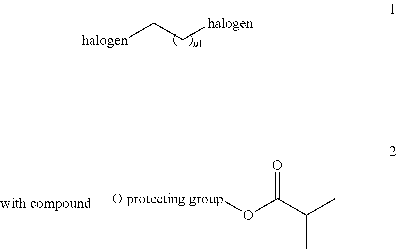

to obtain compound

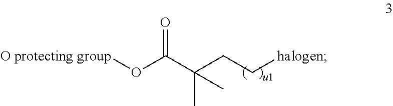

reacting compound 3 with a compound N protecting group —NH₂ to obtain compound

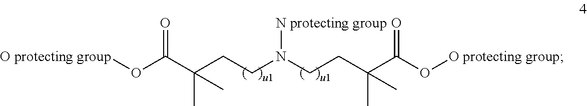

removing O protecting group of compound 4 to obtain compound

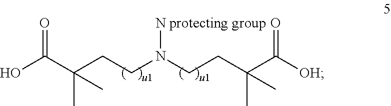

reacting compound 5 with (C(O)-Halogen)₂ to obtain compound

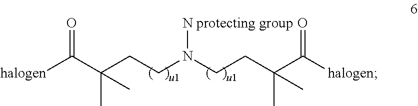

reacting compound 6 with

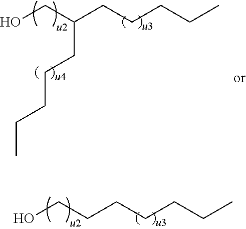

to obtain compound

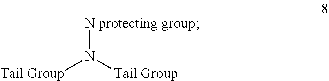

removing N protecting group of compound 8 to obtain compound

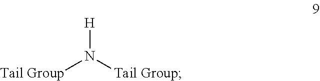

reacting compound 9 with compound

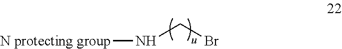

to obtain compound

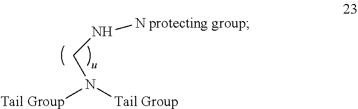

removing N protecting group of compound 23 to obtain compound

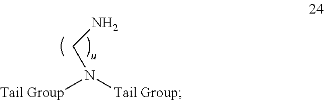

and
reacting compound 24 with compound

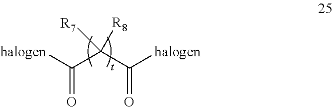

to obtain the lipid having the formula of

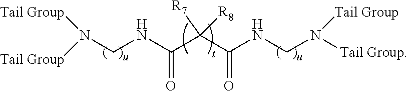

The above process can be shown in the general reaction scheme below:

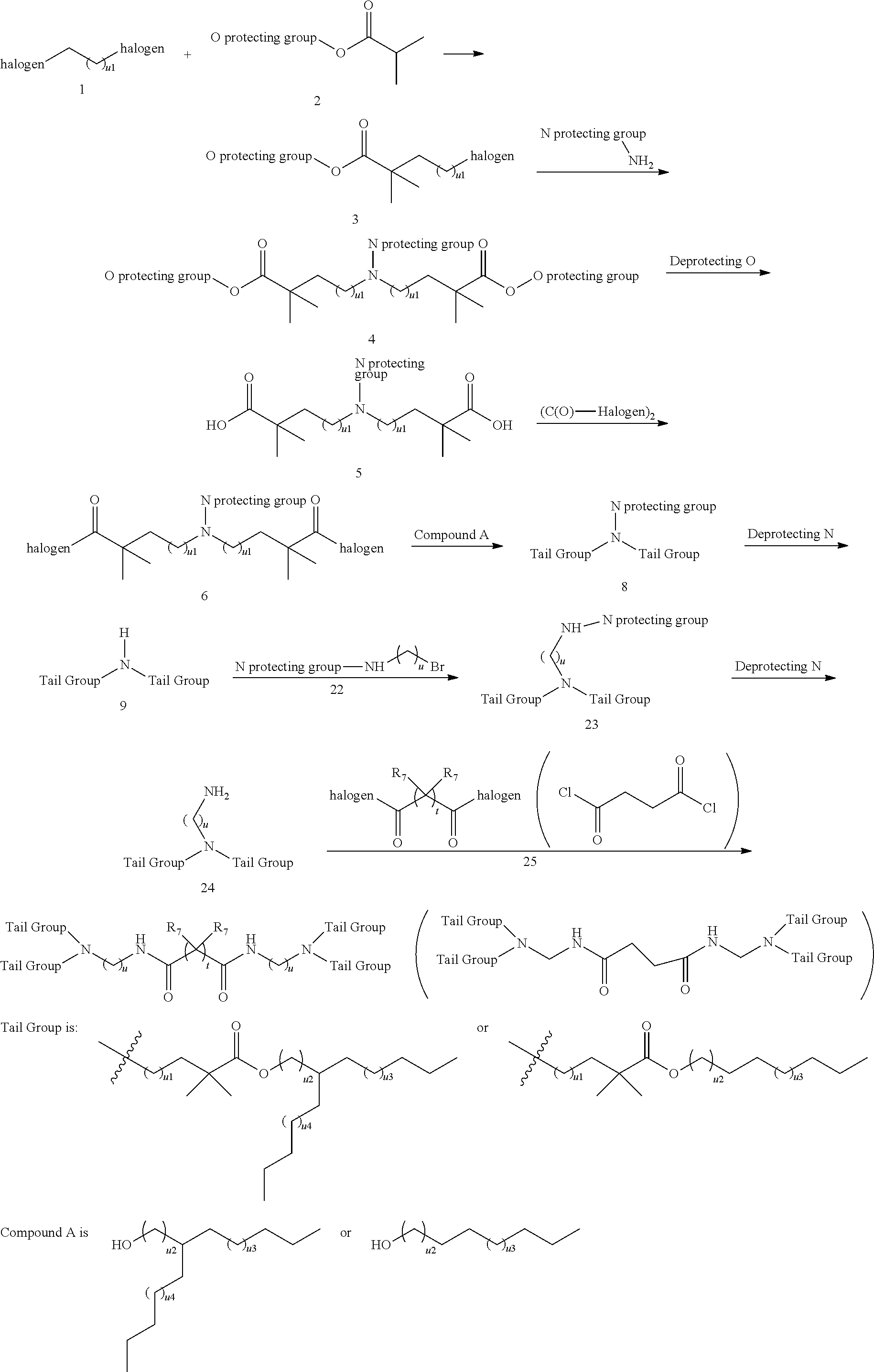

Additional methods of preparing the lipid compounds described herein are exemplified in Examples 1-6 and 9.

### Lipid Composition

Ionizable lipids disclosed herein may be used to form lipid nanoparticle compositions. In some embodiments, the lipid nanoparticle composition further comprises one or more therapeutic agents. In some embodiments, the lipid nanoparticle in the composition encapsulates or is associated with the one or more therapeutic agents.

In some embodiments, the disclosure relates to a composition comprising (i) one or more lipid compounds described herein, comprising at least one head group (e.g., HA-I to HA-VII, HB-I, or HC-I to HC-IIIE; or any subgenus or species of these formulas disclosed herein), and at least one tail group of formula (TI to TIII, or any subgenus or species of these formulas disclosed herein), pharmaceutically acceptable salts thereof, and stereoisomers of any of the foregoing and (ii) one or more lipid component different than the lipid compounds described herein. In some embodiments, the composition comprises 5%, 10%, 15%, 20%, 25%, 30%, 35%, 40%, 45%, 50%, 55%, 60%, 65%, 70%, 75%, 80%, 85%, 90%, or 95% of the one or more lipid compounds.

In some embodiments, the disclosure relates to a composition comprising (i) one or more lipid nanoparticles and (ii) one or more lipid components different than the lipid compounds described herein.

In some embodiments, one or more lipid components different than the lipid compounds described herein comprise one or more helper lipids and one or more PEG lipids. In some embodiments, the lipid component(s) different than the lipid compounds described herein comprise(s) one or more helper lipids, one or more PEG lipids, and one or more neutral lipids.

In some embodiments, the lipid composition may further comprise a sterol and a PEG lipid. In some embodiments, the lipid composition may further comprise a sterol, a PEGylated lipid, a phospholipid, and/or a neutral lipid.

In some embodiments, one or more naturally occurring and/or synthetic lipid compounds may be used in the preparation of the lipid composition. The lipid composition may contain negatively charged lipids, positively charged lipids, or a combination thereof.

### The Non-Ionizable Lipid Components

### Charged and Neutral Lipids

Examples of suitable negatively charged (anionic) lipids include, but are not limited to dimyrystoyl-, dipalmitoyl-, and distearoyl-phasphatidylglycerol; dimyrystoyl-, dipalmitoyl-, and dipalmitoyl-phosphatidic acid; dimyrystoyl-, dipalmitoyl-, and dipalmitoyl-phosphatidylethanolamine; and their unsaturated diacyl and mixed acyl chain counterparts as well as cardiolipin.

Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleyloxypropyl-3-dimethyl-hydroxy ethyl ammonium chloride (DORI), and the cationic lipids described in e.g. Martin et al., Current Pharmaceutical Design, pages 1-394, which is herein incorporated by reference in its entirety.

Additional exemplary cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxy butan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis, cis-9′,12′-octadecadienoxy) propane (CpLin DMA), N,N-Dimethyl-3,4-dioleyloxy benzylamine (DMOBA) and/or a mixture thereof. The neutral lipid can comprise dioleoylphosphatidylethanolamine (DOPE), palmitoyloleoylphosphatidylcholine (POPC), egg phosphatidylcholine (EPC), distearoylphosphatidylcholine (DSPC), and/or a mixture thereof.

In some embodiments, the lipid components comprise one or more neutral lipids. The neutral lipids may be one or more phospholipids, such as one or more (poly) unsaturated lipids. Phospholipids may assemble into one or more lipid bilayers. In general, phospholipids may include a phospholipid moiety and one or more fatty acid moieties. For example, a phospholipid may be a lipid according to formula:

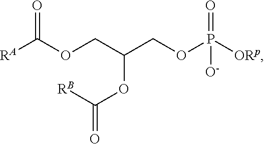

wherein R^{p} represents a phospholipid moiety, and R^{A} and R^{B} represent fatty acid moieties with or without unsaturation that may be the same or different. A phospholipid moiety may be a phosphatidyl choline, phosphatidyl ethanolamine, phosphatidyl glycerol, phosphatidyl serine, phosphatidic acid, 2-lysophosphatidyl choline, or a sphingomyelin. A fatty acid moiety may be a lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, or docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a lipid nanoparticle to facilitate membrane permeation or cellular recognition or in conjugating a lipid nanoparticle to a useful component such as a targeting or imaging moiety (e.g., a dye).

In some embodiments, the neutral lipids may be phospholipids such as distearoyl-sn-glycero-3-phosphocholine (DSPC), 1,2-dioleoyl-sn-gly cero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-gly cero-3-phosphocholine, 1,2-diarachidonoyl-sn-glycero-3-phosphocholine, 1,2-didocosahexaenoyl-sn-glycero-3-phosphocholine, 1,2-diphytanoyl-sn-gly cero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-gly cero-3-phosphoethanolamine, 1,2-didocosahexaenoyl-sn-glycero-3-phosphoethanolamine, 1,2-dioleoyl-sn-gly cero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or mixtures thereof.

Additional non-limiting examples of neutral lipids also include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids may be acyl groups derived from fatty acids having C₁₀-C₂₄ carbon chains, e.g., lauroyl, myristoyl, palmitoyl, stearoyl, or oleoyl.

### Steroids and Other Non-Ionizable Lipid Components

In some embodiments, the lipid components in the lipid composition comprise one or more steroids or analogues thereof.

In some embodiments, the lipid components in the lipid composition comprise sterols such as cholesterol, sisterol and derivatives thereof. Non-limiting examples of cholesterol derivatives include polar analogues such as 5a-cholestanol, 5a-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5a-cholestanone, and cholesteryl decanoate; and mixtures thereof. In some embodiments, the cholesterol derivative is a polar analogue such as cholesteryl-(4′-hydroxy)-butyl ether.

In some embodiments, the non-ionizable lipid components comprises or consists of a mixture of one or more phospholipids and cholesterol or a derivative thereof. In some embodiments, the non-ionizable lipid components comprises or consists of one or more phospholipids, e.g., a cholesterol-free lipid particle formulation. In some embodiments, the non-ionizable lipid components comprises or consists of cholesterol or a derivative thereof, e.g., a phospholipid-free lipid particle formulation.

In some embodiments, the lipid components in the lipid composition (e.g., LNP composition) comprises a phytosterol or a combination of a phytosterol and cholesterol. In some embodiments, the phytosterol is selected from the group consisting of b-sitosterol, stigmasterol, b-sitostanol, campesterol, brassicasterol, and combinations thereof. In some embodiments, the phytosterol is selected from the group consisting of b-sitosterol, b-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S-175 and combinations thereof. In some embodiments, the phytosterol is selected from the group consisting of Compound S-140, Compound S-151, Compound S-156, Compound S-157, Compound S-159, Compound S-160, Compound S-164, Compound S-165, Compound S-170, Compound S-173, Compound S-175, and combinations thereof. In some embodiments, the phytosterol is a combination of Compound S-141, Compound S-140, Compound S-143 and Compound S-148. In some embodiments, the phytosterol comprises a sitosterol or a salt or an ester thereof. In some embodiments, the phytosterol comprises a stigmasterol or a salt or an ester thereof.

In some embodiments, the phytosterol is beta-sitosterol,

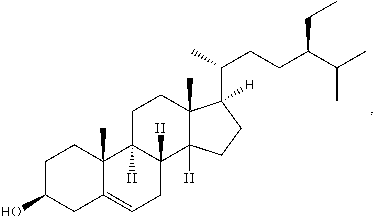

a salt thereof, or an ester thereof.

In some embodiments, the LNP composition comprises a phytosterol, or a salt or ester thereof, and cholesterol or a salt thereof.

In some embodiments, the target cell is a cell described herein (e.g., a liver cell or a splenic cell), and the phytosterol or a salt or ester thereof is selected from the group consisting of b-sitosterol, b-sitostanol, campesterol, and brassicasterol, and combinations thereof. In some embodiments, the phytosterol is b-sitosterol. In some embodiments, the phytosterol is b-sitostanol. In some embodiments, the phytosterol is campesterol. In some embodiments, the phytosterol is brassicasterol.

In some embodiments, the target cell is a cell described herein (e.g., a liver cell or a splenic cell), and the phytosterol or a salt or ester thereof is selected from the group consisting of b-sitosterol, and stigmasterol, and combinations thereof. In some embodiments, the phytosterol is b-sitosterol. In some embodiments, the phytosterol is stigmasterol.

Other examples of non-ionizable lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the non-ionizable lipid comprises from 10 mol % to 60 mol %, from 20 mol % to 55 mol %, from 20 mol % to 45 mol %, 20 mol % to 40 mol %, from 25 mol % to 50 mol %, from 25 mol % to 45 mol %, from 30 mol % to 50 mol %, from 30 mol % to 45 mol %, from 30 mol % to 40 mol %, from 35 mol % to 45 mol %, from 37 mol % to 42 mol %, or 35 mol %, 36 mol %, 37 mol %, 38 mol %, 39 mol %, 40 mol %, 41 mol %, 42 mol %, 43 mol %, 44 mol %, or 45 mol % (or any fraction thereof or range therein) of the total lipid present in the particle.

In embodiments where the lipid particle compositions contain a mixture of phospholipid and cholesterol or a cholesterol derivative, the mixture may comprise up to 40 mol %, 45 mol %, 50 mol %, 55 mol %, or 60 mol % of the total lipid present in the particle.

In some embodiments, the phospholipid component in the mixture may comprise from 2 mol % to 20 mol %, from 2 mol % to 15 mol %, from 2 mol % to 12 mol %, from 4 mol % to 15 mol %, or from 4 mol % to 10 mol % (or any fraction thereof or range therein) of the total lipid present in the particle. In some embodiments, the phospholipid component in the mixture comprises from 5 mol % to 10 mol %, from 5 mol % to 9 mol %, from 5 mol % to 8 mol %, from 6 mol % to 9 mol %, from 6 mol % to 8 mol %, or 5 mol %, 6 mol %, 7 mol %, 8 mol %, 9 mol %, or 10 mol % (or any fraction thereof or range therein) of the total lipid present in the particle.

In some embodiments, the cholesterol component in the mixture may comprise from 25 mol % to 45 mol %, from 25 mol % to 40 mol %, from 30 mol % to 45 mol %, from 30 mol % to 40 mol %, from 27 mol % to 37 mol %, from 25 mol % to 30 mol %, or from 35 mol % to 40 mol % (or any fraction thereof or range therein) of the total lipid present in the particle. In some embodiments, the cholesterol component in the mixture comprises from 25 mol % to 35 mol %, from 27 mol % to 35 mol %, from 29 mol % to 35 mol %, from 30 mol % to 35 mol %, from 30 mol % to 34 mol %, from 31 mol % to 33 mol %, or 30 mol %, 31 mol %, 32 mol %, 33 mol %, 34 mol %, or 35 mol % (or any fraction thereof or range therein) of the total lipid present in the particle.

In embodiments where the lipid particle compositions are phospholipid-free, the cholesterol or derivative thereof may comprise up to 25 mol %, 30 mol %, 35 mol %, 40 mol %, 45 mol %, 50 mol %, 55 mol %, or 60 mol % of the total lipid present in the particle.

In some embodiments, the cholesterol or derivative thereof in the phospholipid-free lipid particle formulation may comprise from 25 mol % to 45 mol %, from 25 mol % to 40 mol %, from 30 mol % to 45 mol %, from 30 mol % to 40 mol %, from 31 mol % to 39 mol %, from 32 mol % to 38 mol %, from 33 mol % to 37 mol %, from 35 mol % to 45 mol %, from 30 mol % to 35 mol %, from 35 mol % to 40 mol %, or 30 mol %, 31 mol %, 32 mol %, 33 mol %, 34 mol %, 35 mol %, 36 mol %, 37 mol %, 38 mol %, 39 mol %, or 40 mol % (or any fraction thereof or range therein) of the total lipid present in the particle.

In some embodiments, the non-ionizable lipid comprises from 5 mol % to 90 mol %, from 10 mol % to 85 mol %, from 20 mol % to 80 mol %, 10 mol % (e.g., phospholipid only), or 60 mol % (e.g., phospholipid and cholesterol or derivative thereof) (or any fraction thereof or range therein) of the total lipid present in the particle.

The percentage of non-ionizable lipid present in the lipid particles is a target amount, and that the actual amount of non-ionizable lipid present in the particle may vary, for example, by ±5 mol %.

A composition containing a ionizable lipid compound may be 30-70% ionizable lipid compound, 0-60% cholesterol, 0-30% phospholipid and 1-10% polyethylene glycol (PEG). In some embodiments, the composition is 30-40% ionizable lipid compound, 40-50% cholesterol, and 10-20% PEG. In some embodiments, the composition is 50-75% ionizable lipid compound, 20-40% cholesterol, and 5-10% phospholipid, and 1-10% PEG. The composition may contain 60-70% ionizable lipid compound, 25-35% cholesterol, and 5-10% PEG. The composition may contain up to 90% ionizable lipid compound and 2-15% helper lipid.

The composition may be a lipid particle composition, for example containing 8-30% compound, 5-30% helper lipid, and 0-20% cholesterol; 4-25% ionizable lipid, 4-25% helper lipid, 2-25% cholesterol, 10-35% cholesterol-PEG, and 5% cholesterol-amine; or 2-30% ionizable lipid, 2-30% helper lipid, 1-15% cholesterol, 2-35% cholesterol-PEG, and 1-20% cholesterol-amine; or up to 90% ionizable lipid and 2-10% helper lipids, or even 100% ionizable lipid.

### Lipid Conjugates

In addition to one or more ionizable lipids, the lipid particles described herein may further comprise one or more lipid conjugates. A conjugated lipid may prevent the aggregation of particles. Non-limiting examples of conjugated lipids include PEG-lipid conjugates, cationic polymer-lipid conjugates, and mixtures thereof.

In some embodiments, the lipid conjugate is a PEG-lipid or PEG-modified lipid (alternatively referred to as PEGylated lipid). A PEG lipid is a lipid modified with polyethylene glycol. Examples of PEG-lipids include, but are not limited to, PEG coupled to dialkyloxypropyls (PEG-DAA), PEG coupled to diacylglycerol (PEG-DAG), PEG-modified dialkylamines, PEG-modified diacylglycerols (PEG-DEG), PEG coupled to phospholipids such as phosphatidylethanolamine (PEG-PE), PEG conjugated to ceramides (PEG-CER), PEG conjugated to cholesterol or a derivative thereof, and mixtures thereof.

For example, a PEG lipid may be PEG-c-DOMG, PEG-DMG, PEG-DLPE, PEG-DMPE, PEG-DPPC, or a PEG-DSPE lipid.

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

In some embodiments, the PEG-lipid is selected from the group consisting of 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-diolexl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

PEG is a linear, water-soluble polymer of ethylene PEG repeating units with two terminal hydroxyl groups. PEGs are classified by their molecular weights; and include the following: monomethoxypoly ethylene glycol (MePEG-OH), monomethoxypoly ethylene glycol-succinate (MePEG-S), monomethoxypoly ethylene glycol-succinimidyl succinate (MePEG-S—NHS), monomethoxypoly ethylene glycol-amine (MePEG-NH₂), monomethoxypoly ethylene glycol-tresylate (MePEG-TRES), monomethoxypoly ethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH₂).

The PEG moiety of the PEG-lipid conjugates described herein may comprise an average molecular weight ranging from 550 daltons to 10,000 daltons. In certain instances, the PEG moiety has an average molecular weight of from 750 daltons to 5,000 daltons (e.g., from 1,000 daltons to 5,000 daltons, from 1,500 daltons to 3,000 daltons, from 750 daltons to 3,000 daltons, from 750 daltons to 2,000 daltons). In some embodiments, the PEG moiety has an average molecular weight of 2,000 daltons or 750 daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In some embodiments, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amido linker moiety). In some embodiments, a carbamate linker is used to couple the PEG to the lipid.

In some embodiments, an ester-containing linker moiety is used to couple the PEG to the lipid. Suitable ester-containing linker moieties include, e.g., carbonate (—OC(O)O—), succinoyl, phosphate esters (—O—(O) POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available, or can be isolated or synthesized using conventional techniques known to those of skill in the art.

In some embodiments, phosphatidylethanolamines contain saturated or unsaturated fatty acids with carbon chain lengths in the range of C10 to C20. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

The term “diacylglycerol” or “DAG” includes a compound having 2 fatty acyl chains, R1 and R2, both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C12), myristoyl (CM), palmitoyl (C16), stearoyl (C18), and icosoyl (C20). In some embodiments, R1 and R2 are the same, i.e., R1 and R2 are both myristoyl (i.e., dimyristoyl), R1 and R2 are both stearoyl (i.e., distearoyl).

The term “dialkyloxy propyl” or “DAA” includes a compound having 2 alkyl chains, R and R′, both of which have independently between 2 and 30 carbons. The alkyl groups can be saturated or have varying degrees of unsaturation.

In some embodiments, the PEG-DAA conjugate is a PEG-didecyloxypropyl (C10) conjugate, a PEG-dilauryloxypropyl (C12) conjugate, a PEG-dimyristyloxypropyl (C14) conjugate, a PEG-dipalmityloxy propyl (C16) conjugate, or a PEG-distearyloxy propyl (C18) conjugate. In some embodiments, the PEG has an average molecular weight of 750 or 2,000 daltons. In some embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and poly dimethylacrylamide, polylactic acid, poly glycolic acid, and derivatized celluloses such as hydroxymethylcellulose or hydroxy ethylcellulose.

In some embodiments, the PEG-lipid is a compound of formula

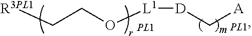

or a salt thereof, wherein:
R^{3PL1} is —OR^{OPL1};
R^{OPL1} is hydrogen, optionally substituted alkyl, or an oxygen protecting group;
r^{PL1} is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{NPL1}), S, C(O), C(O)N(R^{NPL1}), NR^{NFL1}C(O), —C(O)O, OC(O), OC(O)O, OC(O)N(R^{NPL1}), NR^{NPL1}C(O)O, or NR^{NPL1}C(O)N(R^{NPL1});
D is a moiety obtained by click chemistry or a moiety cleavable under physiological conditions; m^{PL1} is 0, 1, 2, 3, 4, 5, 6, 7, 8, 9, or 10;
A is of the formula:

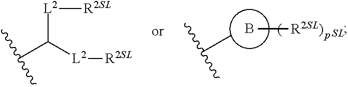

each instance of L² is independently a bond or optionally substituted C₁₋₆ alkylene, wherein one methylene unit of the optionally substituted C₁₋₆ alkylene is optionally replaced with O, N(R^{NPL1}), S, C(O), C(O)N(R^{NPL1}), NR^{NPL1}C(O), C(O)O, OC(O), OC(O)O, —OC(O)N(R^{NPL1}), NR^{NPL1}C(O)O, or NR^{NPL1}C(O)N(R^{NPL1});
each instance of R^{2SL} is independently optionally substituted C₁₋₃₀ alkyl, optionally substituted C₁₋₃₀ alkenyl, or optionally substituted C₁₋₃₀ alkynyl; optionally wherein one or more methylene units of R^{2SL} are independently replaced with optionally substituted carbocyclylene, optionally substituted heterocyclylene, optionally substituted arylene, optionally substituted heteroarylene, N(R^{NPL1}), O, S, C(O), C(O)N(R^{NPL1}), NR^{NPL1}C(O), —NR^{NPL1}C(O)N(R^{NPL1}), C(O)O, OC(O), OC(O)O, OC(O)N(R^{NPL1}), NR^{NPL1}C(O)O, C(O)S, —SC(O), C(═NR^{NPL1}), C(═NR^{NPL1})N(R^{NPL1}), NR^{NPL1}C(═NR^{NPL1}), —NR^{NPL1}C(═NR^{NPL1})N(R^{NPL1}), C(S), C(S)N(R^{NPL1}), NR^{NPL1}C(S), NR^{NPL1}C(S)N(R^{NPL1}), S(O), OS(O), S(O)O, OS(O)O, OS(O)₂, S(O)₂O, OS(O)₂O, N(R^{NPL1})S(O), S(O)N(R^{NPL1}), —N(R^{NPL1})S(O)N(R^{NPL1}), OS(O)N(R^{NPL1}), N(R^{NPL1})S(O)O, S(O)₂, N(R^{NPL1})S(O)₂, —S(O)₂N(R^{NPL1}), N(R^{NPL1})S(O)₂N(R^{NPL1}), OS(O)₂N(R^{NPL1}), or N(R^{NPL1})S(O)₂O;
each instance of R^{NPL1} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p^{SL} is 1 or 2.

In some embodiments, the PEG-lipid is a compound of formula

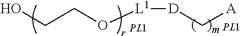

or a salt thereof, wherein r^{PL1}, L¹, D, m^{PL1}, and A are as above defined.

In some embodiments, the PEG-lipid is a compound of formula

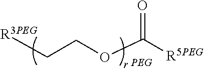

or a salt or isomer thereof, wherein:
R^{3PEG} is —OR^{O};
R^{O} is hydrogen, C₁₋₆ alkyl or an oxygen protecting group;
r^{PEG} is an integer between 1 and 100 (e.g., between 40 and 50, e.g., 45);
R^{5PEG} is C₁₀₋₄₀ alkyl (e.g., C₁₇ alkyl), C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{NPEG})—, —O—, —S—, —C(O)—, —C(O)N(R^{NPEG})—, —NR^{NPEG}C(O)—, —NR^{NPEG}C(O)N(R^{NPEG})—, —C(O)O—, —OC(O)—, —OC(O)O—, —OC(O)N(R^{NPEG})—, —NR^{NPEG}C(O)O—, —C(O)S—, —SC(O)—, —C(═NR^{NPEG})—, —C(═NR^{NPEG})N(R^{NPEG})—, —NR^{NPEG}C(═NR^{NPEG})—, —NR^{NPEG}C(═NR^{NPEG})N(R^{NPEG})—, —C(S)—, —C(S)N(R^{NPEG})—, —NR^{NPEG}C(S)—, —NR^{NPEG}C(S)N(R^{NPEG})—, —S(O)—, —OS(O)—, —S(O)O—, —OS(O)O—, —OS(O)₂—, —S(O)₂O—, —OS(O)₂O—, —N(R^{NPEG})S(O)—, —S(O)N(R^{NPEG})—, —N(R^{NPEG})S(O)N(R^{NPEG})—, —OS(O)N(R^{NPEG})—, —N(R^{NPEG})S(O)O—, —S(O)₂—, —N(R^{NPEG})S(O)₂—, —S(O)₂N(R^{NPEG})—, —N(R^{NPEG})S(O)₂N(R^{NPEG})—, —OS(O)₂N(R^{NPEG})—, or —N(R^{NPEG})S(O)₂O—; and
each instance of R^{NPEG} is independently hydrogen, C₁₋₆ alkyl, or a nitrogen protecting group.

In some embodiments, the PEG-lipid is a compound of formula

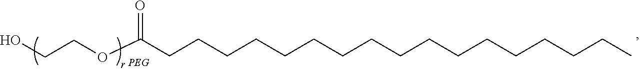

wherein r^{PEG} is an integer between 1 and 100 (e.g., between 40 and 50, e.g., 45).

In some embodiments, the PEG-lipid is a compound of formula

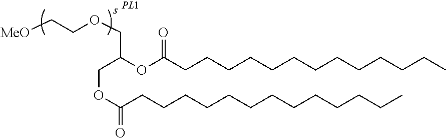

or a salt or isomer thereof, wherein s^{PL1} is an integer between 1 and 100 (e.g., between 40 and 50, e.g., 45).

In some embodiments, the PEG-lipid has the formula of

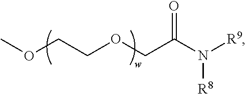

or a pharmaceutically acceptable salt, tautomer or stereoisomer thereof, wherein:
R⁸ and R⁹ are each independently a straight or branched, saturated or unsaturated alkyl chain containing from 10 to 30 carbon atoms, wherein the alkyl chain is optionally interrupted by one or more ester bonds (e.g., R⁸ and R⁹ are each independently straight, saturated alkyl chains containing from 12 to 16 carbon atoms); and
w has a mean value ranging from 30 to 60 (e.g., the average w is about 49).

In some embodiments, the incorporation of any of the above-discussed PEG-lipids in the lipid composition can improve the pharmacokinetics and/or biodistribution of the lipid composition. For example, incorporation of any of the above-discussed PEG-lipids in the lipid composition can reduce the accelerated blood clearance (ABC) effect.

### Other Iniozable Lipids

In some embodiments, the lipid composition may comprise one or more additional ionizable lipids, different than the ionizable lipids described herein. Exemplary ionizable lipids include, but are not limited to,

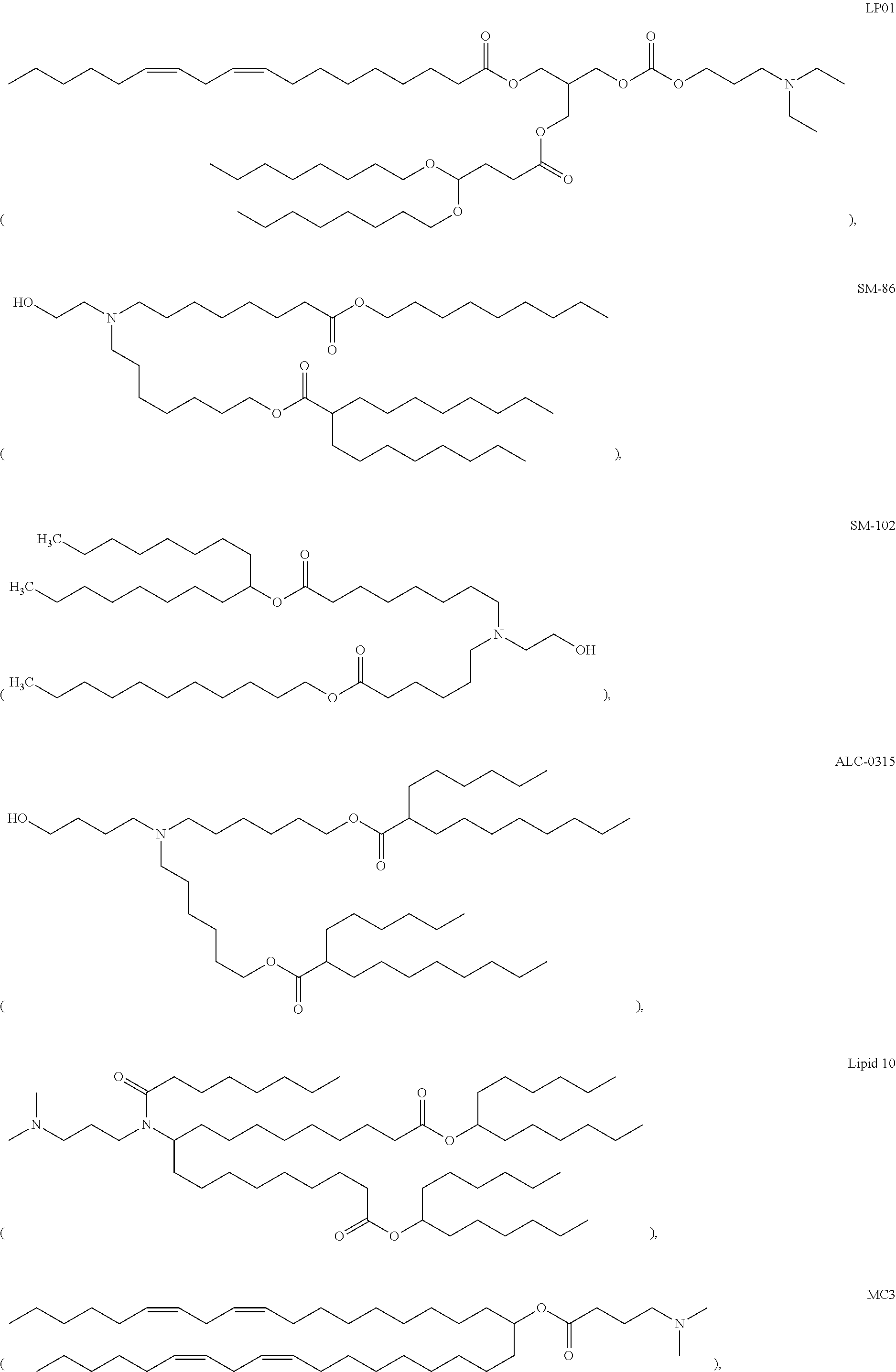

Acuitas Lipid 9, and Acuitas Lipid 10 (see WO 2017/004143A1, which is incorporated herein by reference in its entirety).

In one embodiment, the additional ionizable lipid is heptadecan-9-yl 8-((2-hydroxyethyl) (6-oxo-6-(undecyloxy)hexyl)amino)octanoate (SM-102); e.g., as described in Example 1 of U.S. Pat. No. 9,867,888 (which is incorporated by reference herein in its entirety).

In one embodiment, the additional ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO 2015/095340 (which is incorporated by reference herein in its entirety).

In one embodiment, the additional ionizable lipid is Di((Z)-non-2-en-1-yl) 9-((4-dimethylamino)butanoyl)oxy)heptadecanedioate (L319), e.g., as synthesized in Example 7, 8, or 9 of US 2012/0027803 (which is incorporated by reference herein in its entirety).

In one embodiment, the additional ionizable lipid is 1,1′-((2-(4-(2-((2-(Bis(2-hydroxydodecyl)amino)ethyl)(2-hydroxydodecyl)amino)ethyl)piperazin-1-yl)ethyl)azanediyl)bis(dodecan-2-ol)(C12-200), e.g., as synthesized in Examples 14 and 16 of WO 2010/053572 (which is incorporated by reference herein in its entirety).

In one embodiment, the additional ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl) propanoate, e.g., Structure (I) from WO 2020/106946 (which is incorporated by reference herein in its entirety).

In one embodiment, the additional ionizable lipid is MC3 (6Z,9Z,28Z,3 1Z)-heptatriaconta-6,9,28,31-tetraen-19-yl-4-(dimethylamino) butanoate (DLin-MC3-DMA or MC3), e.g., as described in Example 9 of WO 2019/051289A9, which is incorporated by reference herein in its entirety.

In one embodiment, the additional ionizable lipid is lipid ATX-002, e.g., as described in Example 10 of WO 2019/051289A9, which incorporated by reference herein in its entirety.

In one embodiment, the additional ionizable lipid is is (13Z,16Z)-A,A-dimethyl-3-nonyldocosa-13, 16-dien-1-amine (Compound 32), e.g., as described in Example 11 of WO 2019/051289A9 (which is incorporated by reference herein in its entirety).

In one embodiment, the additional ionizable lipid is Compound 6 or Compound 22, e.g., as described in Example 12 of WO 2019/051289A9, which is incorporated by reference herein in its entirety.

Examples of additional ionizable lipids useful in the lipid composition include those listed in Table 1 of WO 2019/051289, which is incorporated herein by reference.

### Additional Lipid Components

Some non-limiting examples of additional lipid compounds that may be used (e.g., in combination with the ionizable lipid compound described herein and other lipid components) to form the lipid composition include:

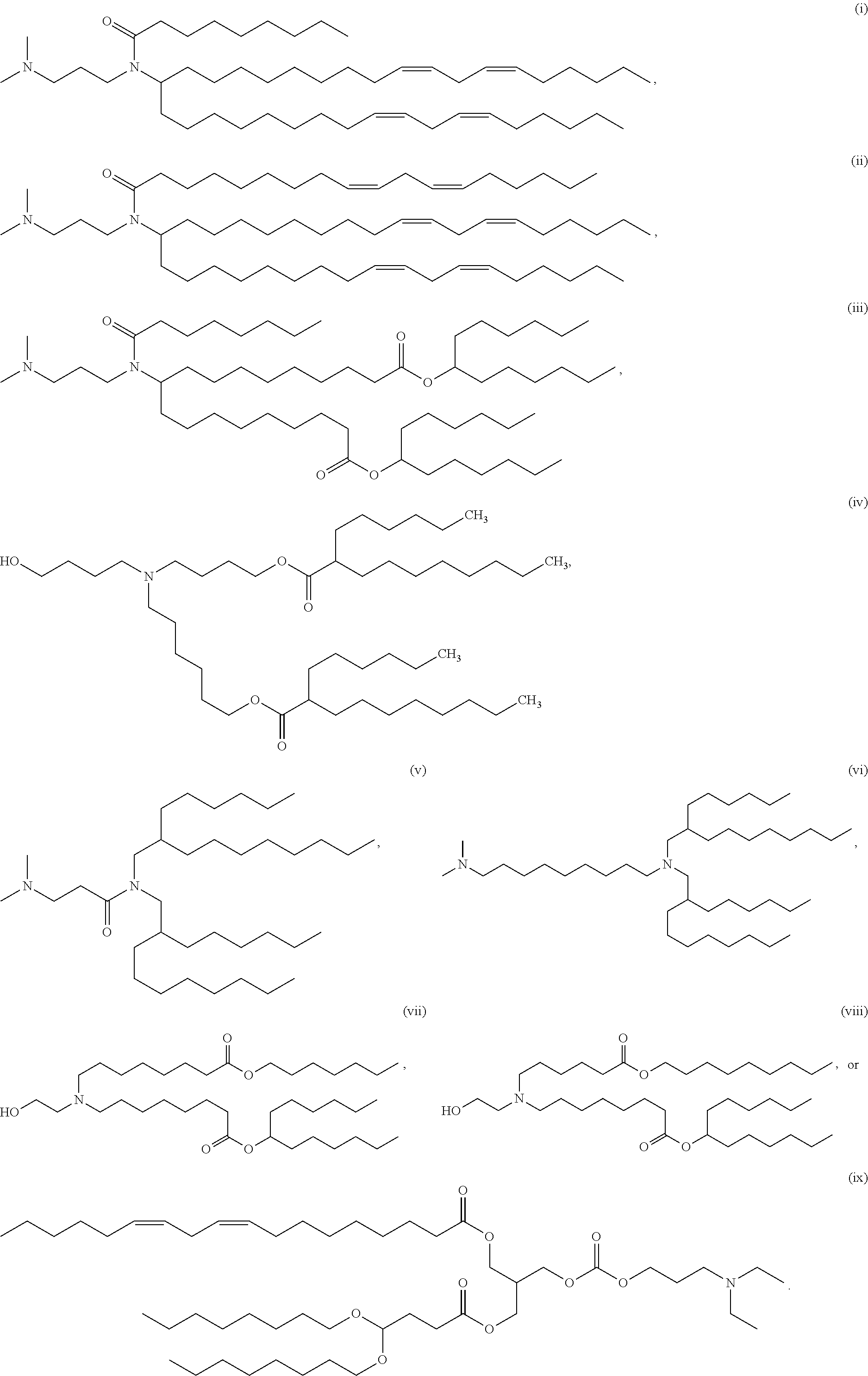

In some embodiments, the lipid composition further comprises the lipids in formula (i), (ii), (iii), (iv), (v), (vi), (vii), (viii), or (ix).

In some embodiments, the lipid composition further comprises the following compounds having the structure of:

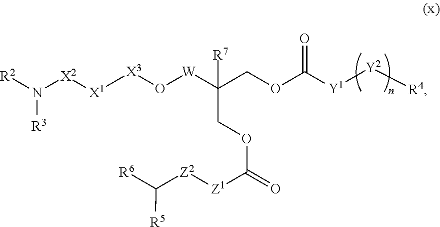

wherein:
   X¹ is O, NR¹, or a direct bond, X² is C₂₋₅ alkylene, and X³ is C(═O) or a direct bond;
   R¹ is H or Me, R³ is C₁₋₃ alkyl, R² is C₁₋₃ alkyl, or R² taken together with the nitrogen atom to which it is attached and 1-3 carbon atoms of X² form a 4-, 5-, or 6-membered ring; or
   X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring;
   Y¹ is C₂₋₁₂ alkylene, and Y² is selected from

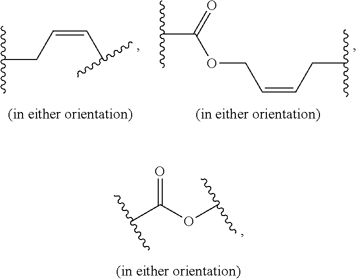 [SMILES: */C=C\C* ; *C/C=C\COC(*)=O ; *OC(*)=O]

n is 0 to 3;
R⁴ is C₁-15 alkyl;
Z¹ is C₁₋₆ alkylene or a direct bond, and Z² is

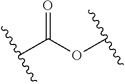 [SMILES: *OC(*)=O]

(in either orientation) or absent, provided that if Z¹ is a direct bond, Z² is absent;
R⁵ is C₅₋₉ alkyl or C₆₋₁₀ alkoxy, R⁶ is C₅₋₉ alkyl or C₆₋₁₀ alkoxy;
W is methylene or a direct bond; and
R⁷ is H or Me, or a salt thereof;
provided that if R³ and R² are C₂ alkyls, X¹ is O, X² is linear C₃ alkylene, X³ is C(═O), Y¹ is linear C₅ alkylene, (Y²)n-R⁴ is:

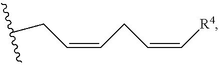

R⁴ is linear C₅ alkyl, Z¹ is C₂ alkylene, Z² is absent, W is methylene, and R⁷ is H, then R⁵ and R⁶ are not C₂ alkoxy. In some embodiments, the lipid composition further comprises one or more compounds of formula (x).

Additional non-limiting examples of lipid compounds that may be further included in the lipid composition further comprises (e.g., in combination with the lipid compounds described herein and other lipid components):

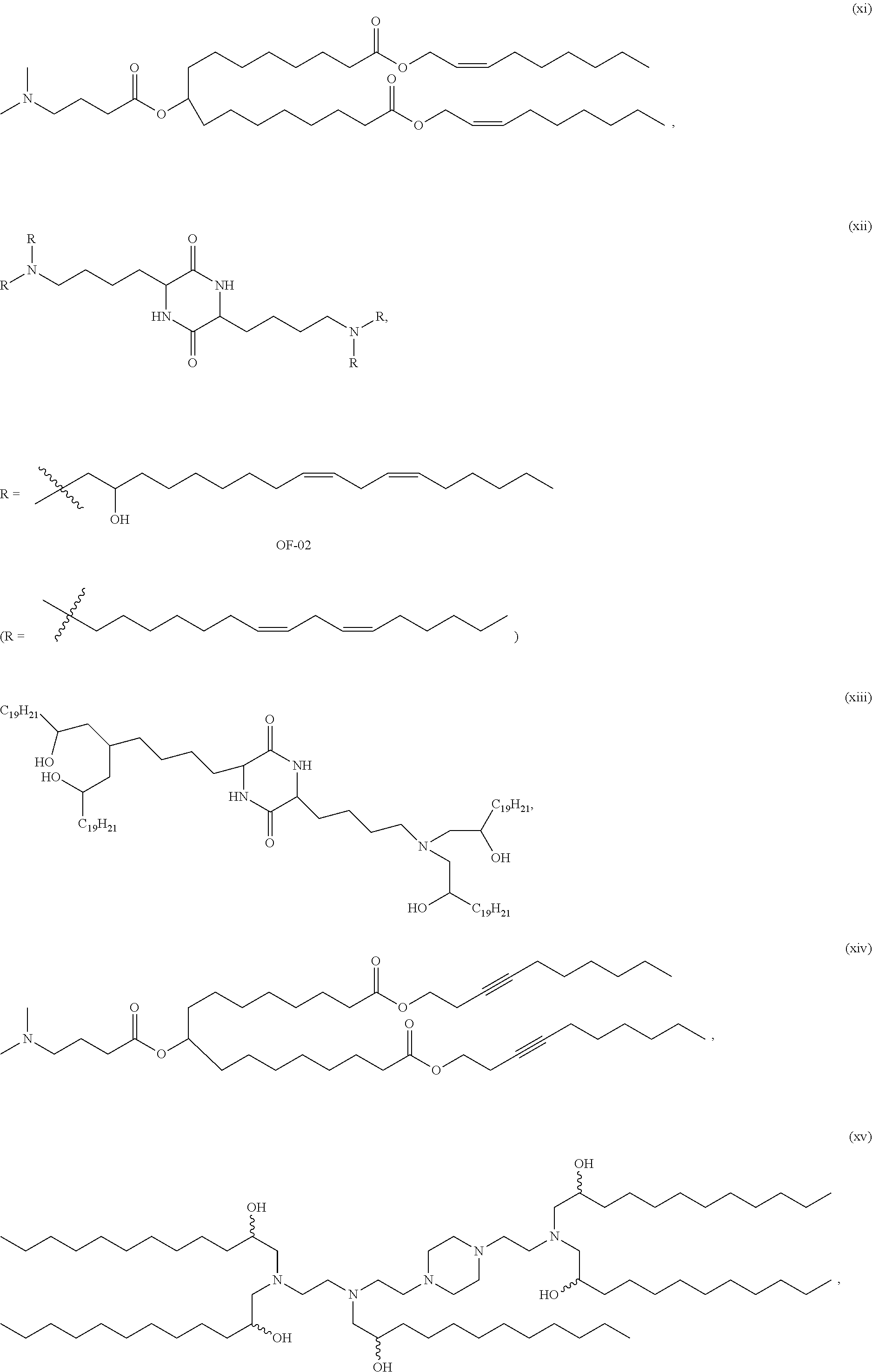

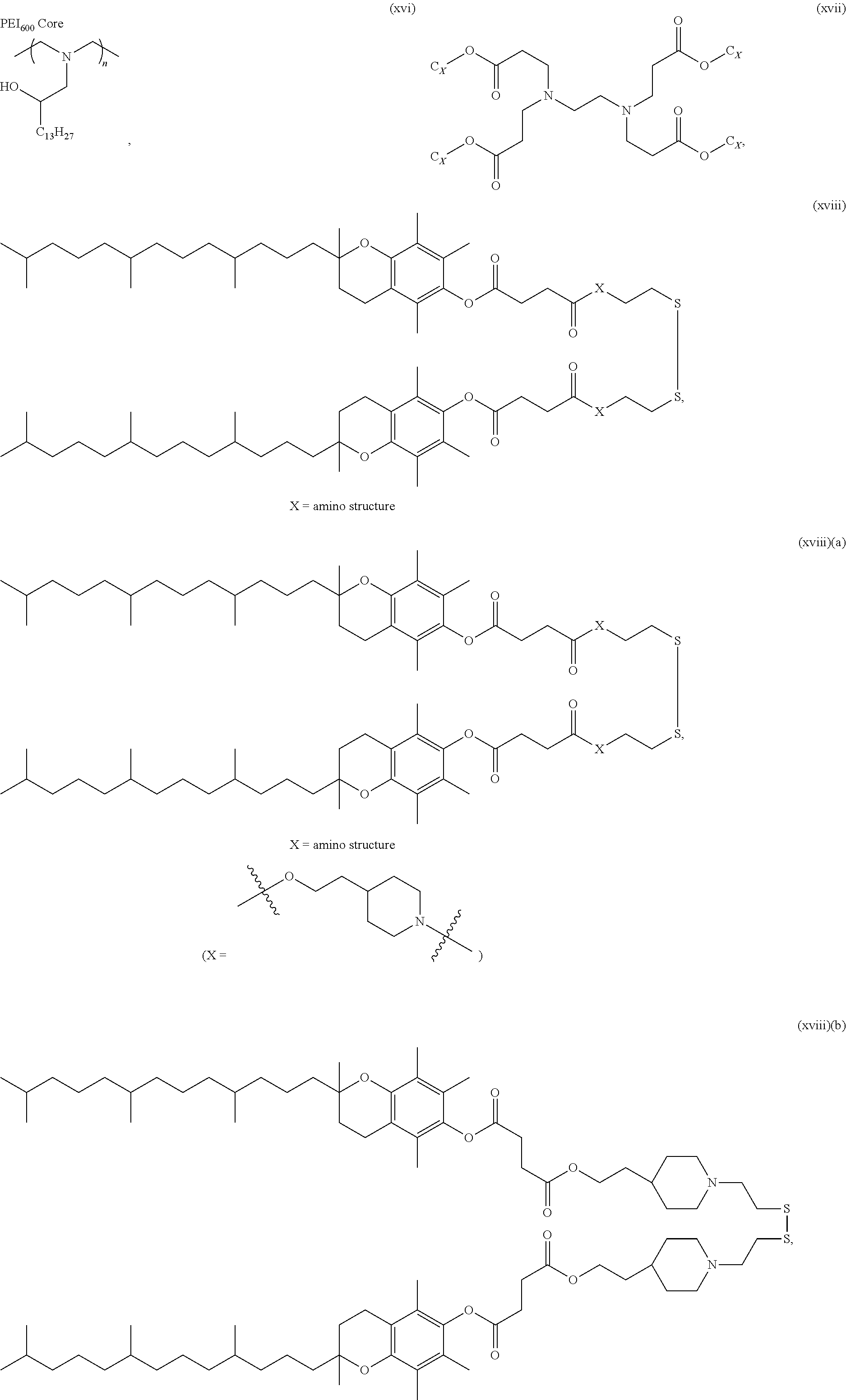

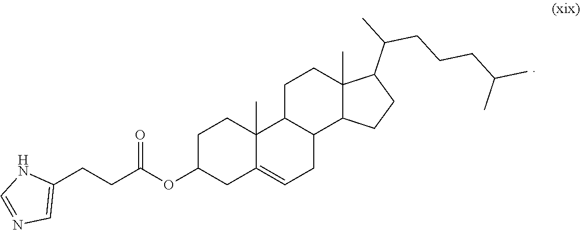 [SMILES: CC(C)CCCC(C)C1CCC2C3CC=C4CC(OC(=O)CCc5cnc[nH]5)CCC4(C)C3CCC12C]

In some embodiments, the lipid composition further comprises one or more compounds of formula (xi), (xii), (xiii), (xiv), (xv), (xvi), (xvii), (xviii) (e.g., (xviii)a, (xviii)b), or (xix).

In some embodiments, the lipid composition further comprises lipids formed by one of the following reactions:

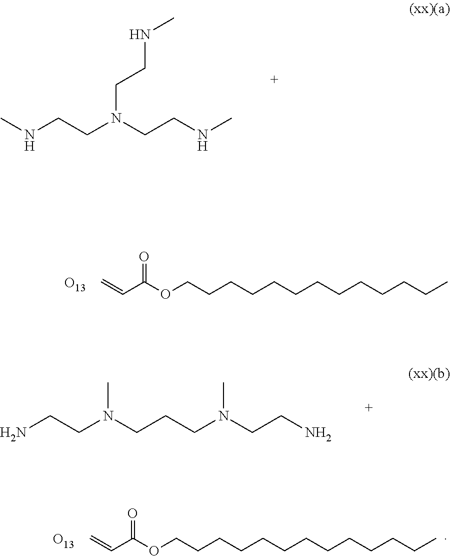

In some embodiments, the lipid composition further comprises the lipid (e.g., in combination with the lipid compounds described herein and other lipid components) having the formula (xxi):

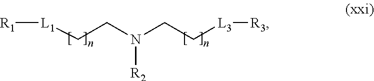

wherein:
each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
   R₂ is selected from a group consisting of:

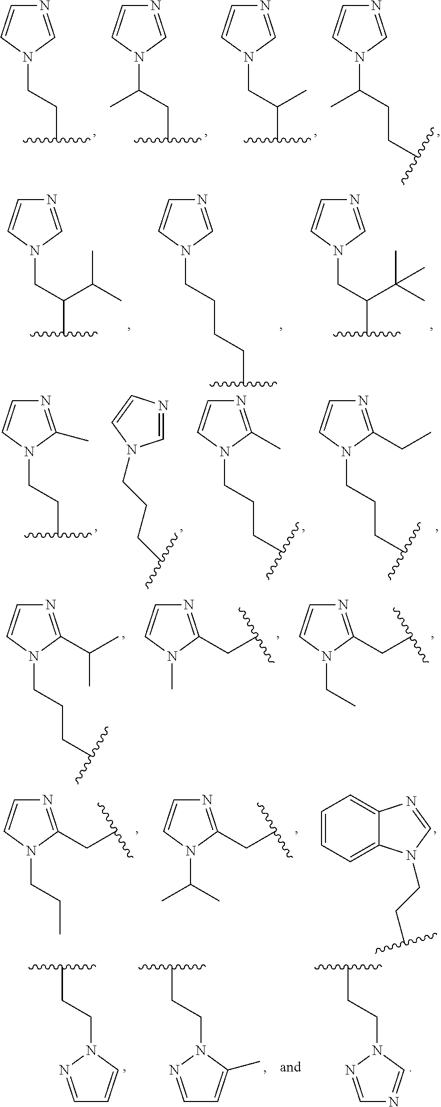

In some embodiments, the lipid composition further comprises one or more compounds of formula (xxi). In some embodiments, the compounds of formula (xxi) include those described by WO 2021/113777 (e.g., a lipid of Formula (1) such as a lipid of Table 1 of WO 2021/113777), which is incorporated herein by reference in its entirety.

In some embodiments, the lipid composition further comprises lipids (e.g., in combination with the lipid compound described herein and other lipid components) having the formula (xxii):

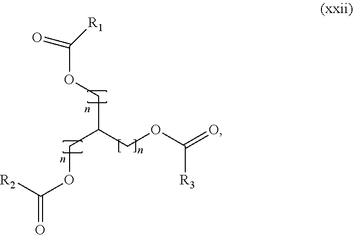

wherein:
each n is independently an integer from 1-15;
R₁ and R₂ are each independently selected from a group consisting of:

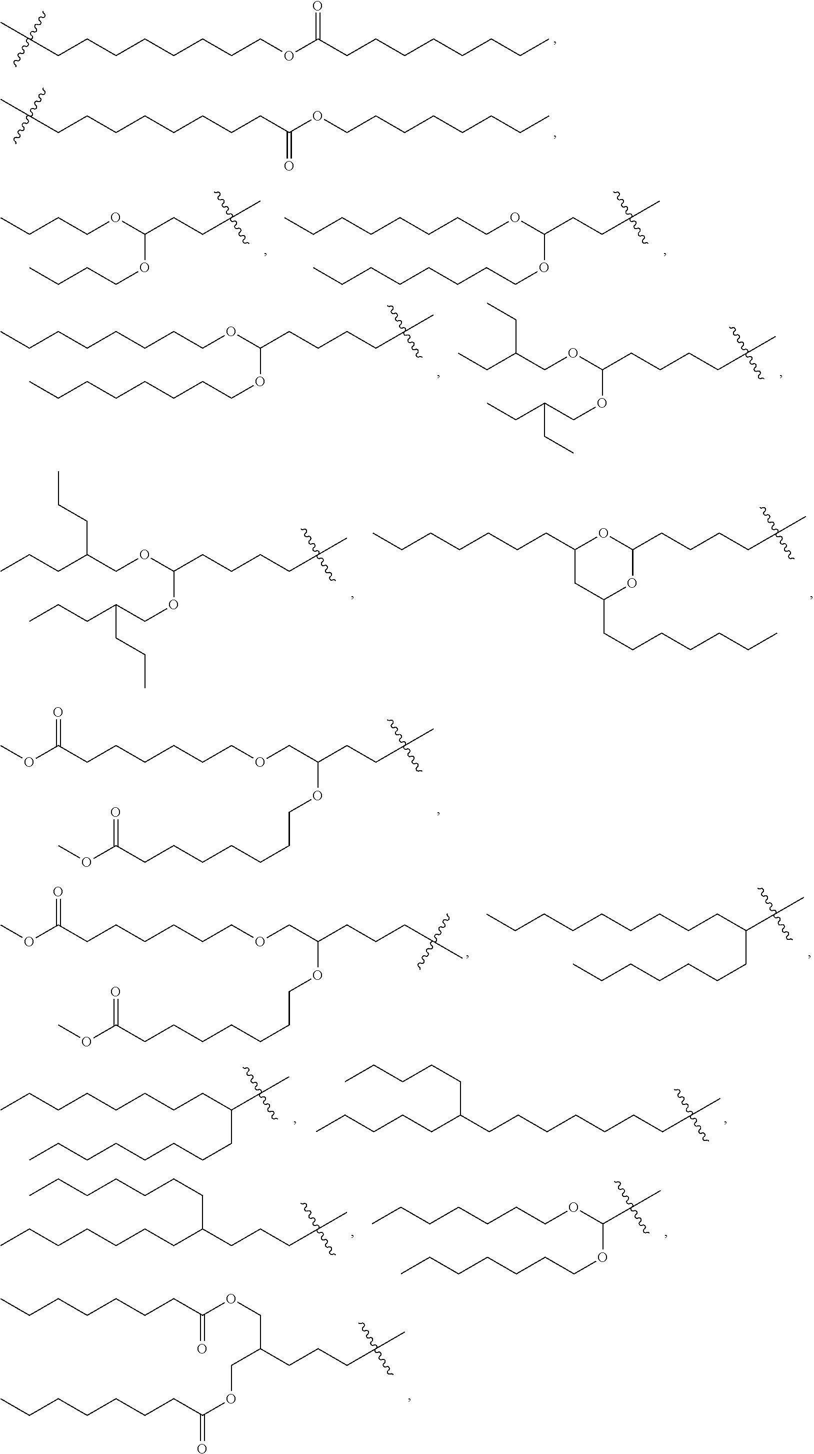

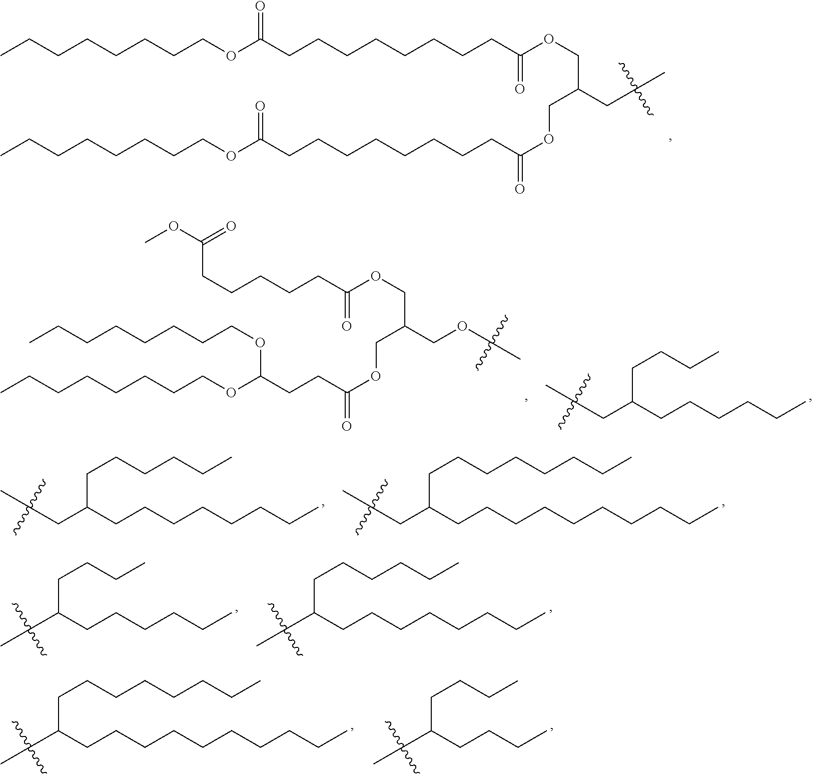

R₃ is selected from a group consisting of:

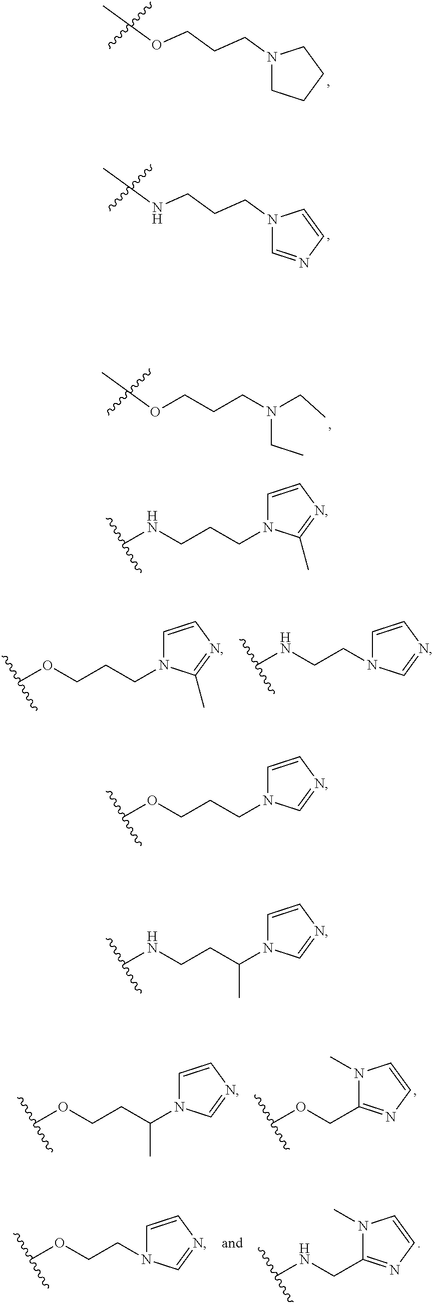

In some embodiments, the lipid composition further comprises one or more compounds of formula (xxii). In some embodiments, the compounds of formula (xxii) include those described by WO 2021/113777 (e.g., a lipid of Formula (2) such as a lipid of Table 2 of WO 2021/113777), which is incorporated herein by reference in its entirety.

In some embodiments, the lipid composition further comprises lipids (e.g., in combination with the lipid compound described herein and other lipid components) having the formula (xxiii):

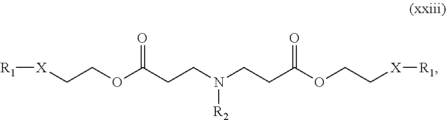

wherein
X is selected from —O—, —S—, or —OC(O)—*, wherein * indicates the attachment point to R₁;
R₁ is selected from a group consisting of:

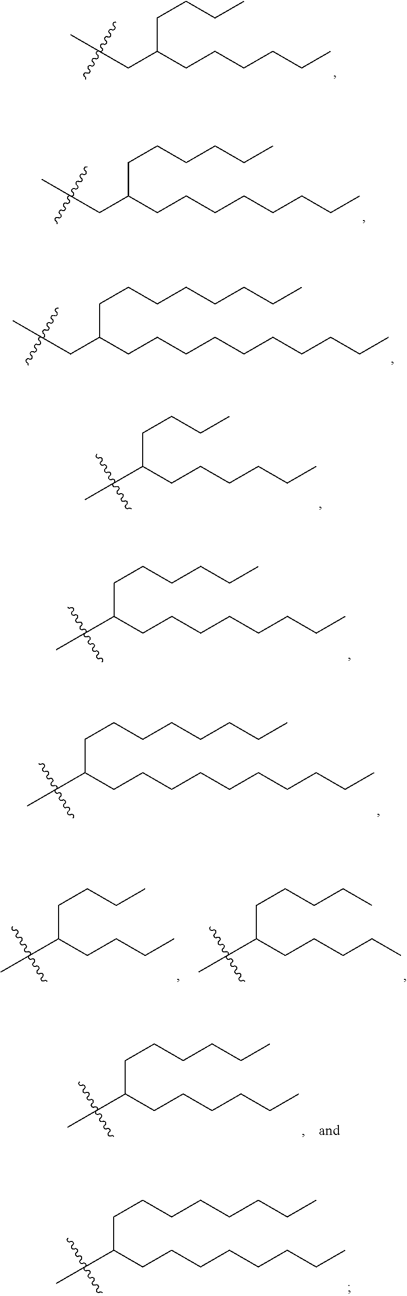

and
R₂ is selected from a group consisting of:

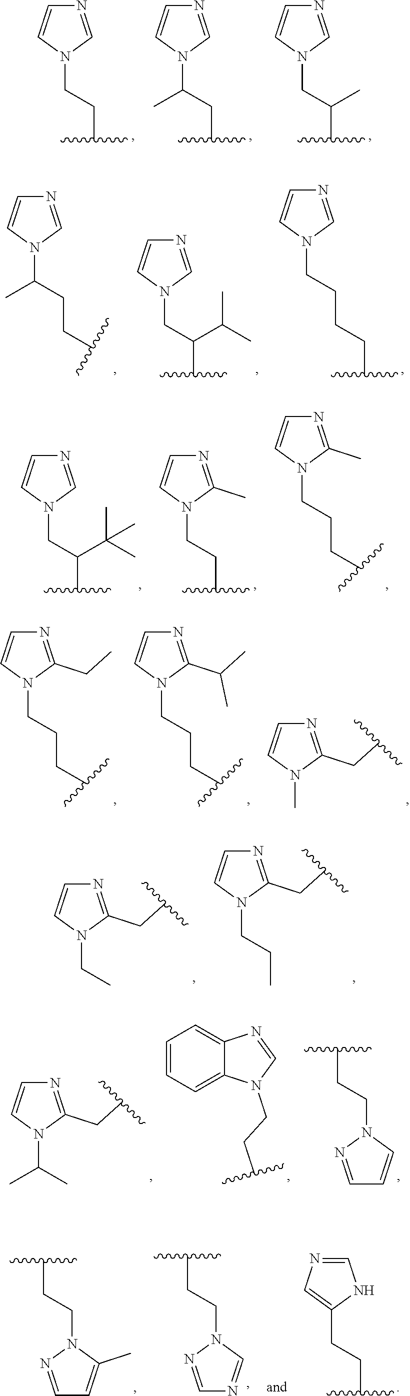

In some embodiments, the lipid composition further comprises one or more compounds of formula (xxiii). In some embodiments, the compounds of formula (xxiii) include those described by WO 2021/113777 (e.g., a lipid of Formula (3) such as a lipid of Table 3 of WO 2021/113777), which is incorporated herein by reference in its entirety.

Examples of additional lipids that can be used in the lipid composition include, without limitation, one or more of the following formulas: X of US 2016/0311759; I of US 20150376115 or in US 2016/0376224; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210); I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of US 2013/0123338; I or X-A-Y—Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; 1-5 or 1-8 of WO 2020/081938; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-O13 or 503-O13 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid conjugate (e.g., PEG-lipid) comprises from 0.1 mol % to 2 mol %, from 0.5 mol % to 2 mol %, from 1 mol % to 2 mol %, from 0.6 mol % to 1.9 mol %, from 0.7 mol % to 1.8 mol %, from 0.8 mol % to 1.7 mol %, from 0.9 mol % to 1.6 mol %, from 0.9 mol % to 1.8 mol %, from 1 mol % to 1.8 mol %, from 1 mol % to 1.7 mol %, from 1.2 mol % to 1.8 mol %, from 1.2 mol % to 1.7 mol %, from 1.3 mol % to 1.6 mol %, or from 1.4 mol % to 1.5 mol % (or any fraction thereof or range therein) of the total lipid present in the particle. In some embodiments, the lipid conjugate (e.g., PEG-lipid) comprises from 0 mol % to 20 mol %, from 0.5 mol % to 20 mol %, from 2 mol % to 20 mol %, from 1.5 mol % to 18 mol %, from 2 mol % to 15 mol %, from 4 mol % to 15 mol %, from 2 mol % to 12 mol %, from 5 mol % to 12 mol %, or 2 mol % (or any fraction thereof or range therein) of the total lipid present in the particle.

In further embodiments, the lipid conjugate (e.g., PEG-lipid) comprises from 4 mol % to 10 mol %, from 5 mol % to 10 mol %, from 5 mol % to 9 mol %, from 5 mol % to 8 mol %, from 6 mol % to 9 mol %, from 6 mol % to 8 mol %, or 5 mol %, 6 mol %, 7 mol %, 8 mol %, 9 mol %, or 10 mol % (or any fraction thereof or range therein) of the total lipid present in the particle.

The percentage of lipid conjugate (e.g., PEG-lipid) present in the lipid particles of the disclosure is a target amount, and the actual amount of lipid conjugate present in the composition may vary, for example, by ±2 mol %. One of ordinary skill in the art will appreciate that the concentration of the lipid conjugate can be varied depending on the lipid conjugate employed and the rate at which the lipid particle is to become fusogenic.

By controlling the composition and concentration of the lipid conjugate, one can control the rate at which the lipid conjugate exchanges out of the lipid particle and, in turn, the rate at which the lipid particle becomes fusogenic. In addition, other variables including, e.g., pH, temperature, or ionic strength, can be used to vary and/or control the rate at which the lipid particle becomes fusogenic. Other methods which can be used to control the rate at which the lipid particle becomes fusogenic will become apparent to those of skill in the art upon reading this disclosure. Also, by controlling the composition and concentration of the lipid conjugate, one can control the lipid particle size.

In some embodiments, the composition further comprises one or more nucleic acids, ionizable lipids, amphiphiles, phospholipids, cholesterol, and/or PEG-linked cholesterol.

### Other Components for the LNP Composition

The lipid nanoparticle composition may include one or more components in addition to those described above. For example, a LNP composition may include one or more small hydrophobic molecules such as a vitamin (e.g., vitamin A or vitamin E) or a sterol.

The lipid nanoparticle composition may also include one or more permeability enhancer molecules, carbohydrates, polymers, surface altering agents, or other components.

Suitable carbohydrates may include simple sugars (e.g., glucose) and polysaccharides (e.g., glycogen and derivatives and analogs thereof).

A polymer may be used to encapsulate or partially encapsulate a nanoparticle composition. The polymer may be biodegradable and/or biocompatible. Suitable polymers include, but are not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid)(PLA), poly(L-lactic acid) (PLLA), poly(glycolic acid)(PGA), poly(lactic acid-co-glycolic acid)(PLGA), poly(L-lactic acid-co-glycolic acid)(PLLGA), poly(D,L-lactide)(PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D, L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene (PS), polyurethanes, derivatized celluloses such as alkyl celluloses, hydroxyalkyl celluloses, cellulose ethers, cellulose esters, nitro celluloses, hydroxy propylcellulose, carboxymethylcellulose, polymers of acrylic acids, such as poly(methyl(meth)acrylate)(PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxy methylene, poloxamers, polyoxamines, poly(ortho)esters, poly(butyric acid), poly(valeric acid), poly(lactide-co-caprolactone), trimethylene carbonate, poly(N-acryloylmorpholine)(PAcM), poly(2-methyl-2-oxazoline)(PMOX), poly(2-ethyl-2-oxazoline)(PEOZ), and polyglycerol.

Suitable surface altering agents include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymnosin β4, dornase alfa, neltenexine, and erdosteine), and DNases (e.g., rhDNase). A surface altering agent may be disposed within a lipid nanoparticle and/or on the surface of a lipid nanoparticle (e.g., by coating, adsorption, covalent linkage, or other process).

The lipid nanoparticle composition may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a lipid nanoparticle may also be conjugated with one or more useful antibodies. Functional groups and conjugates useful in targeted cell delivery, imaging, and membrane permeation are well known in the art.

The lipid nanoparticle composition may include any substance useful in pharmaceutical compositions. For example, the lipid nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included.

Suitable diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxy methyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Suitable surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN® 20], polyoxyethylene sorbitan [TWEEN® 60], polyoxyethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g., polyoxyethylene monostearate [MYRJ® 45], polyoxyethylene hydrogenated castor oil, polyethoxylated castor oil, polyoxy methylene stearate, and SOLUTOL®), sucrose fatty acid esters, polyethylene glycol fatty acid esters (e.g. CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

Suitable binding agents may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; morganic calcium salts; silicic acid; polymethacrylates; waxes; water; alcohol; and combinations thereof, or any other suitable binding agent.

Suitable preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine chlorobutanol, chlorocresol, chloroxylenol, cresol, ethyl alcohol, glycerin, hexidine, imidurea, phenol, phenoxyethanol, phenylethyl alcohol, phenylmercuric nitrate, propylene glycol, and/or thimerosal Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Suitable lubricating agents include, but are not limited to, magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Suitable oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

In some embodiments, the composition further comprises one or more cryoprotectants. Suitable cryoprotective agents include, but are not limited to, a polyol (e.g., a diol or a triol such as propylene glycol (i e, 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG₂ₖ-DMG, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium acetate, sodium chloride, sodium formate, sodium malonate, sodium nitrate, sodium sulfate, or any hydrate thereof), or any combination thereof.

In some embodiments, the cryoprotectant comprises sucrose. In some embodiments, the cryoprotectant and/or excipient is sucrose. In some embodiments, the cryoprotectant comprises sodium acetate. In some embodiments, the cryoprotectant and/or excipient is sodium acetate. In some embodiments, the cryoprotectant comprises sucrose and sodium acetate.

In some embodiments, the composition further comprises one or more buffers. Suitable buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the buffer is an acetate buffer, a citrate buffer, a phosphate buffer, a tris buffer, or combinations thereof.

### Pharmaceutical Compositions

Another aspect of the disclosure also provides pharmaceutical compositions comprising the lipid composition as described herein, which comprises one or more lipid compounds chosen from an ionizable lipid compound described herein, and a pharmaceutically acceptable excipient. The pharmaceutical composition may further comprise a therapeutic agent.

All above descriptions and all embodiments discussed in the above aspects relating to the aspects of the lipid compounds, and the exemplary variables and compounds are all applicable to these aspects of the invention relating to the pharmaceutical composition.

All above descriptions and all embodiments discussed in the above aspects relating to the aspects of the lipid composition, including various other lipid components, are applicable to these aspects of the invention relating to the pharmaceutical composition.

In the lipid composition containing the therapeutic agent, the ratio of total lipid components to the cargo (e.g., an encapsulated therapeutic agent such as a nucleic acid) can be varied as desired. For example, the total lipid components to the cargo (mass or weight) ratio can be from about 10:1 to about 30:1. In some embodiments, the total lipid components to the cargo ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the cargo can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid composition's overall lipid content can range from about 5 mg/ml to about 30 mg/mL.

### Therapeutic Agents

### Nucleic Acid Molecule

In some embodiments, the composition further comprises one or more nucleic acid components. The nucleic acid molecule may be a plasmid, an immunostimulatory oligonucleotide, an antisense oligonucleotide, an antagomir, an aptamer, a deoxyribozyme (DNAzyme), and a ribozyme.

In some embodiments, the composition further comprises one or more RNA and/or DNA components.

In some embodiments, the composition further comprises one or more DNA components. In some embodiments, the DNA is linear DNA, circular DNA, single stranded DNA, or double stranded DNA.

In some embodiments, the composition further comprises one or more RNA components. In some embodiments, the RNA is mRNA, miRNA, siRNA, RNA aptamer, linear RNA, circular RNA, single stranded RNA, double stranded RNA, tRNA, microRNA (miRNA) or miRNA precursor, a Dicer substrate small interfering RNA (dsiRNA), a short hairpin RNA (shRNA), an asymmetric interfering RNA (aiRNA), a guide RNA (gRNA), lncRNA, ncRNA, sncRNA, rRNA, snRNA, piRNA, snoRNA, snRNA, scaRNA, exRNA, scaRNA, Y RNA, or hnRNA.

In some embodiments, the one or more RNA components is chosen from mRNA. In some embodiments, the mRNA is a modified mRNA.

In some embodiments, the nucleic acid molecule is an enzymatic nucleic acid molecule. The term “enzymatic nucleic acid molecule” refers to a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity.

In some embodiments, the nucleic acid molecule is an antisense nucleic acid. The term “antisense nucleic acid” refers to a non-enzymatic nucleic acid molecule that binds to target RNA by means of RNA-RNA or RNA-DNA or RNA-PNA (protein nucleic acid) interactions and alters the activity of the target RNA.

In some embodiments, the nucleic acid molecule may be a 2-5A antisense chimera. The term “2-5A antisense chimera” refers to an antisense oligonucleotide containing a 5′-phosphorylated 2′-5′-linked adenylate residue.

In some embodiments, the nucleic acid molecule may be a triplex forming oligonucleotide. The term “triplex forming oligonucleotide” refers to an oligonucleotide that can bind to a double-stranded DNA in a sequence-specific manner to form a triple-strand helix.

In some embodiments, the nucleic acid molecule may be a decoy RNA. The term “decoy RNA” refers to a RNA molecule or aptamer that is designed to preferentially bind to a predetermined ligand. Such binding can result in the inhibition or activation of a target molecule.

In some embodiments, the nucleic acid molecule (e.g., RNA or DNA) encodes a therapeutic peptide or polypeptide, operably linked to a promoter for a DNA. The therapeutic peptide or polypeptide may be, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase. Cpf/Cas12a); a Crispr-linked enzyme, e.g., a base editor or prime editor; a mobile genetic element protein (e.g., a transposase, a retrotransposase, a recombinase, an integrase); a Gene Writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In some embodiments, the one or more RNA components comprise a gRNA nucleic acid. In some embodiments, the gRNA nucleic acid is a gRNA.

In some embodiments, the one or more RNA components comprise a Class 2 Cas nuclease mRNA and a gRNA. In some embodiments, the gRNA nucleic acid is or encodes a dual-guide RNA (dgRNA). In some embodiments, the gRNA nucleic acid is or encodes a single-guide RNA (sgRNA). In some embodiments, the gRNA is a modified gRNA. In some embodiments, the modified gRNA comprises a modification at one or more of the first five nucleotides at a 5° end. In some embodiments, the modified gRNA comprises a modification at one or more of the last five nucleotides at a 3′ end.

In some embodiments, the one or more RNA components comprise an mRNA. In some embodiments, the one or more RNA components comprise an RNA-guided DNA-binding agent, for example a Cas nuclease mRNA (such as a Class 2 Cas nuclease mRNA) or a Cas9 nuclease mRNA.

All the nucleic acid molecules described herein can be chemically modified. The various modification strategy to the nucleic acid molecules are well known to one skilled in the art. In some embodiments, the nucleic acid molecule comprises one or more modifications selected from the group consisting of pseudouridine, 5-bromouracil, 5-methylcytosine, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or fluorescein linked to the sugar), thiol containing nucleotides, biotin linked nucleotides, fluorescent base analogs. CpG islands, methyl-7-guanosine, methylated nucleotides, inosine, thiouridine, pseudourdine, dihydrouridine, queuosine, and wyosine. In some embodiments, the antisense oligonucleotide may be a locked nucleic acid oligonucleotide (LNA). The term “locked nucleic acid (LNA)” refers to oligonucleotides that contain one or more nucleotide building blocks in which an extra methylene bridge fixes the ribose moiety either in the C3′-endo (beta-D-LNA) or C2′-endo (alpha-L-LNA) conformation (Grunweller A, Hartmann R K, BioDrugs, 21(4):235-243 (2007)).

In some embodiments, the composition further comprises one or more template nucleic acids. Additional examples of the nucleic acid molecules (including tumor suppressor genes, antisense oligonucleotides, siRNA, miRNA, or shRNA) may be found in U.S. Published Patent Application No. 2007/0065499 and U.S. Pat. No. 7,780,882, which are incorporated by reference herein in their entireties.

In some embodiments, the pharmaceutical composition can include a plurality of nucleic acid molecules, which may be the same or different types.

Nucleic acids for use with embodiments of this disclosure may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g., including but not limited to that from the T7, T3 and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012).

Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see, e.g. Losick, R., 1972, In vitro transcription, Ann Rev Biochem v. 41 409-46; Kamakaka, R. T, and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N. Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed mRNA may be purified from the undesired components of the transcription or associated reactions (including unincorporated rNTPs, protein enzyme, salts, short RNA oligos, etc.). Techniques for the isolation of the mRNA transcripts are well known in the art. Well known procedures include, for non-limiting examples, phenol/chloroform extraction or precipitation with either alcohol (ethanol, isopropanol) in the presence of monovalent cations or lithium chloride.

Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v. 10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek).

Furthermore, while reverse transcription can yield large quantities of mRNA, the products can contain a number of aberrant RNA impurities associated with undesired polymerase activity which may need to be removed from the full-length mRNA preparation. These include short RNAs that result from abortive transcription initiation as well as double-stranded RNA (dsRNA) generated by RNA-dependent RNA polymerase activity. RNA-primed transcription from RNA templates and self-complementary 3′ extension. It has been demonstrated that these contaminants with dsRNA structures can lead to undesired immunostimulatory activity through interaction with various innate immune sensors in eukaryotic cells that function to recognize specific nucleic acid structures and induce potent immune responses. This in turn, can dramatically reduce mRNA translation since protein synthesis is reduced during the innate cellular immune response. Therefore, additional techniques to remove these dsRNA contaminants have been developed and are known in the art including but not limited to scaleable HPLC purification (see, e.g., Kariko, K., Muramatsu, H., Ludwig, J. And Weissman, D., 2011. Generating the optimal mRNA for therapy: HPLC purification eliminates immune activation and improves translation of nucleoside-modified, protein-encoding mRNA. Nucl Acid Res, v. 39 e142; Weissman, D., Pardi, N., Muramatsu, H., and Kariko, K., HPLC Purification of in vitro transcribed long RNA in Synthetic Messenger RNA and Cell Metabolism Modulation in Methods in Molecular Biology v. 969 (Rabinovich, P. H. Ed), 2013). HPLC purified mRNA has been reported to be translated at much greater levels, particularly in primary cells and in vivo.

A significant variety of modifications have been described in the art which are used to alter specific properties of in vitro transcribed mRNA, and may improve its utility. These include, but are not limited to modifications to the 5′ and 3′ termini of the mRNA. Endogenous eukaryotic mRNA typically contain a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. The conjugated guanine nucleotide is methylated at the N7 position. Additional modifications include methylation of the ultimate and penultimate most S′-nucleotides on the 2′-hydroxyl group.

Multiple distinct cap structures can be used to generate the 5′-cap of in vitro transcribed synthetic mRNA. 5′-capping of synthetic mRNA can be performed co-transcriptionally with chemical cap analogs (i.e., capping during in vitro transcription). For example, the Anti-Reverse Cap Analog (ARC A) cap contains a 5′-5′-triphosphate guanine-guanine linkage where one guanine contains an N7 methyl group as well as a 3′-0-methyl group. However, up to 20% of transcripts remain uncapped during this co-transcriptional process and the synthetic cap analog is not identical to the 5′-cap structure of an authentic cellular mRNA, potentially reducing translatability and cellular stability. Alternatively, synthetic miRNA molecules may also be enzymatically capped post-transcriptionally. These may generate a more authentic 5′-cap structure that more closely mimics, either structurally or functionally, the endogenous 5′-cap which have enhanced binding of cap binding proteins, increased half-life and reduced susceptibility to 5′ endonucleases and/or reduced 5′ decapping. Numerous synthetic 5′-cap analogs have been developed and are known in the art to enhance mRNA stability and translatability (see, e.g., Grudzien-Nogalska, E., Kowalska, J., Su, W., Kuhn, A. N., Slepenkov, S. V., Darynkiewicz, E., Sahin, U., Jemielity. J., and Rhoads, R. E., Synthetic mRNAs with superior translation and stability properties in Synthetic Messenger RNA and Cell Metabolism Modulation in Methods in Molecular Biology v. 969 (Rabinovich, P. H. Ed), 2013).

On the 3′-terminus, a long chain of adenine nucleotides (poly-A tail) is normally added to mRNA molecules during RNA processing. Immediately after transcription, the 3′ end of the transcript is cleaved to free a 3′ hydroxyl to which poly-A polymerase adds a chain of adenine nucleotides to the RNA in a process called polyadenylation. The poly-A tail has been extensively shown to enhance both translational efficiency and stability of mRNA (see Bernstein, P, and Ross, J., 1989, Poly (A), poly (A) binding protein and the regulation of mRNA stability, Trends Bio Sci v. 14 373-377; Guhaniyogi, J. And Brewer, G., 2001, Regulation of mRNA stability in mammalian cells, Gene, v. 265, 11-23; Dreyfus, M. And Regnier, P., 2002, The poly (A) tail of mRNAs: Bodyguard in eukaryotes, scavenger in bacteria, Cell, v. I1, 611-613).

Poly (A) tailing of in vitro transcribed mRNA can be achieved using various approaches including, but not limited to, cloning of a poly (T) tract into the DNA template or by post-transcriptional addition using Poly (A) polymerase. The first case allows in vitro transcription of mRNA with poly (A) tails of defined length, depending on the size of the poly (T) tract, but requires additional manipulation of the template. The latter case involves the enzymatic addition of a poly (A) tail to in vitro transcribed mRNA using poly (A) polymerase which catalyzes the incorporation of adenine residues onto the 3′ termini of RNA, requiring no additional manipulation of the DNA template, but results in mRNA with poly (A) tails of heterogeneous length. 5′-capping and 3′-poly (A) tailing can be performed using a variety of commercially available kits including, but not limited to Poly (A) Polymerase Tailing kit (EpiCenter), mMESSAGE mMACHINE T7 Ultra kit and Poly (A) Tailing kit (Life Technologies) as well as with commercially available reagents, various ARCA caps, Poly (A) polymerase, etc.

In addition to 5′ cap and 3′ poly adenylation, other modifications of the in vitro transcripts have been reported to provide benefits as related to efficiency of translation and stability. It is well known in the art that pathogenic DNA and RNA can be recognized by a variety of sensors within eukaryotes and trigger potent innate immune responses. The ability to discriminate between pathogenic and self DNA and RNA has been shown to be based, at least in part, on structure and nucleoside modifications since most nucleic acids from natural sources contain modified nucleosides. In contrast, in vitro synthesized RNA lacks these modifications, thus rendering it immunostimulatory which in turn can inhibit effective mRNA translation as outlined above. The introduction of modified nucleosides into in vitro transcribed mRNA can be used to prevent recognition and activation of RNA sensors, thus mitigating this undesired immunostimulatory activity and enhancing translation capacity (see, e.g., Kariko, K. And Weissman, D. 2007, Naturally occurring nucleoside modifications suppress the immunostimulatory activity of RNA: implication for therapeutic RNA development, Curr Opin Drug Discov Devel, v. 10 523-532; Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013; Kariko, K., Muramatsu, H., Welsh, F. A., Ludwig, J., Kato, H., Akira, S., Weissman, D., 2008, Incorporation of Pseudouridine Into mRNA Yields Superior Nonimmunogenic Vector With Increased Translational Capacity and Biological Stability, Mol Ther v. 16, 1833-1840). The modified nucleosides and nucleotides used in the synthesis of modified RNAs can be prepared monitored and utilized using general methods and procedures known in the art. A large variety of nucleoside modifications are available that may be incorporated alone or in combination with other modified nucleosides to some extent into the in vitro transcribed mRNA (see, e.g., US2012/0251618). In vitro synthesis of nucleoside-modified mRNA has been reported to have reduced ability to activate immune sensors with a concomitant enhanced translational capacity.

Other components of mRNA which can be modified to provide benefit in terms of translatability and stability include the 5′ and 3′ untranslated regions (UTR). Optimization of the UTRs (favorable 5′ and 3′ UTRs can be obtained from cellular or viral RNAs), either both or independently, have been shown to increase mRNA stability and translational efficiency of in vitro transcribed mRNA (see, e.g., Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013).

In addition to mRNA, other nucleic acid payloads may be used for this disclosure. For oligonucleotides, methods of preparation include but are not limited to chemical synthesis and enzymatic, chemical cleavage of a longer precursor, in vitro transcription as described above, etc. Methods of synthesizing DNA and RNA nucleotides are widely used and well known in the art (see, e.g., Gait, M. J. (ed.) Oligonucleotide synthesis: a practical approach, Oxford [Oxfordshire], Ishington, D. C.: IRL Press. 1984; and Herdewijn. P. (ed.) Oligonucleotide synthesis: methods and applications. Methods in Molecular Biology, v. 288 (Clifton, N.J.) Totowa, N.J.: Humana Press, 2005; both of which are incorporated herein by reference).

For plasmid DNA, preparation for use with embodiments of this disclosure commonly utilizes, but is not limited to, expansion and isolation of the plasmid DNA in vitro in a liquid culture of bacteria containing the plasmid of interest. The presence of a gene in the plasmid of interest that encodes resistance to a particular antibiotic (penicillin, kanamycin, etc.) allows those bacteria containing the plasmid of interest to selectively grow in antibiotic-containing cultures. Methods of isolating plasmid DNA are widely used and well known in the art (see, e.g., Heilig, J., Elbing, K. L. and Brent, R., (2001). Large-Scale Preparation of Plasmid DNA, Current Protocols in Molecular Biology, 41:11:1.7:1.7.1-1.7.16; Rozkov, A., Larsson, B., Gillstrom, S., Bjornestedt, R, and Schmidt, S. R., (2008), Large-scale production of endotoxin-free plasmids for transient expression in mammalian cell culture, Biotechnol. Bioeng., 99:557-566; and U.S. Pat. No. 6,197,553 B1). Plasmid isolation can be performed using a variety of commercially available kits including, but not limited to Plasmid Plus (Qiagen), GenJET plasmid MaxiPrep (Thermo) and Pure Yield MaxiPrep (Promega) kits as well as with commercially available reagents.

In some embodiments, the lipid nanoparticle compositions are useful for expression of protein encoded by mRNA. In some embodiments, provided herein are methods for expression of protein encoded by mRNA.

In some embodiments, the LNP composition has an N/P ratio of from about 1:1 to about 30:1, for instance, from about 3:1 to about 20:1, from about 3:1 to about 15:1, from about 3:1 to about 10:1, or from about 3:1 to about 6:1. For example, the N/P ratio of the nucleic acid molecule-encapsulated lipid composition may be about 6±1, or the N/P ratio of the nucleic acid molecule-encapsulated lipid composition may be about 6±0.5. In some embodiments, the N/P ratio of the nucleic acid molecule-encapsulated lipid composition ranges from about 3:1 to about 15:1. In some embodiments, the N/P ratio of the nucleic acid molecule-encapsulated lipid composition is about 6. An N:P ratio refers to the molar ratio of the amines present in the lipid composition or lipid nanoformulation (e.g., the amines in the ionizable lipids) to the phosphates present in the nucleic acid molecule. It is a factor for efficient packaging and potency.

### Other Therapeutic Agents

The therapeutic agent can be a peptide or protein, a small molecule drug, encapsulated in the lipid composition. The pharmaceutical composition can contain two or more different therapeutic agents from the nucleic acid molecule, peptide or protein, and small molecule drug.

In some embodiments, the protein may be a peptide or polypeptide, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g., a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In some embodiments, the pharmaceutical composition can include a plurality of protein molecules, which may be the same or different types.

In some embodiments, the therapeutic agent is a small molecule drug, for instance, a small molecule drug approved for use in humans by an appropriate regulatory authority.

In some embodiments, the pharmaceutical composition can include a plurality of small molecule drugs, which may be the same or different types.

In some embodiments, the therapeutic agent is a vaccine. In some embodiments, the vaccine is a RNA vaccine, such as a RNA cancer vaccine or RNA vaccine for infectious disease (e.g., an influenza virus vaccine or a corona virus vaccine (e.g., COVID-19 vaccine).

### Other Ingredients

The pharmaceutical compositions may contain one or more pharmaceutically acceptable excipients. The pharmaceutically acceptable excipient is selected on the basis of the mode and route of administration. Suitable pharmaceutical carriers or excipients for use in pharmaceutical formulations are described in Remington: The Science and Practice of Pharmacy, 21^{st} Ed., Gennaro, Ed., Lippencott Williams & Wilkins (2005); Handbook of Pharmaceutical Excipients, 6^{th} Edition, Rowe et al., Eds., Pharmaceutical Press (2009); and the USP/NF (United States Pharmacopeia and the National Formulary), which are herein incorporated by reference in their entirety.

In some embodiments, the pharmaceutically acceptable excipient includes one or more of an antioxidant, binder, antiadherent, buffer, coloring agent, diluent (e.g., solid or liquid), disintegrant (e.g., coatings disintegrate), dispersing agent, dyestuff, filler, emulsifier, flavoring agent, lubricant, pH adjuster, pigment, preservative, stabilizer, solubilizing agent, solvent, suspending agent, sweetener, or wetting agent, or combination thereof.

Examples of suitable excipients include, without limitation, acacia, alginate, calcium phosphate, calcium carbonate, calcium silicate, carbopol gel, carboxymethyl cellulose, carnauba wax, cellulose, crospovidone, dextrose, diacetylated monoglycerides, ethylcellulose, gelatin, glyceryl monostearate 40-50, gum acacia, gum arabic, hydroxyethyl cellulose, hydroxypropyl cellulose, hydroxypropylmethyl cellulose, hypromellose phthalate, hypromellose, lactose, lecithin, magnesium stearate, kaolin, methacrylic acid copolymer type C, mannitol, methyl cellulose, methylhydroxybenzoate, microcrystalline cellulose, povidone, polyethylene glycol, polysorbate 80, polyvinylpyrrolidone, propylhydroxy benzoate, sodium carboxymethyl cellulose sodium hydroxide, sodium stearyl fumarate, sodium starch glycolate, starch, sorbitan monooleate sorbitol, sorbic acid, sucrose, talc, tragacanth, talc, triethyl citrate, titanium dioxide, yellow ferric oxide, talc, oil medium (e.g., peanut oil, liquid paraffin, mineral oil, olive oil, almond oil, glycerin, propylene glycol), or water,

When the excipient serves as a diluent, it can be a solid, semisolid, or liquid material (e.g., normal saline), which acts as a vehicle, carrier or medium for the active ingredient. As is known in the art, the type of diluent can vary depending upon the intended route of administration.

The pharmaceutical compositions can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers such as phosphate, citrate, and other organic acids; antioxidants including ascorbic acid and methionine, preservatives (such as octadecyldimethylbenzyl chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) poly peptides; proteins, such as serum albumin, gelatin, or immunoglobulins; hydrophilic polymers such as polyvinylpyrrolidone; amino acids such as gly cine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium, metal complexes (e.g., Zn-protein complexes); and/or nonionic surfactants such as TWEEN™, PLURONICS™ or polyethylene glycol (PEG).

Suitable carriers or excipients for the pharmaceutical compositions may also include a substance that enhances the ability of the body of an individual to absorb the LNP or liposome. Suitable carriers and/or excipients also include any substance that can be used to bulk up formulations with a LNP or liposome, to allow for convenient and accurate dosage. In addition, carriers and/or excipients may be used in the manufacturing process to aid in the handling of a LNP or liposome. Depending on the route of administration, and form of medication, different carriers and/or excipients may be used.

Carriers and/or excipients may also include vehicles and/or diluents. “Vehicles” indicates any of various media acting usually as solvents or carriers; “diluent” indicates a diluting agent which is issued to dilute an active ingredient of a composition; suitable diluent include any substance that can decrease the viscosity of a medicine. The type and amounts of carriers and/or excipients are chosen in function of the chosen pharmaceutical form; suitable pharmaceutical forms are liquid systems like solutions, infusions, suspensions; semisolid systems like colloids, gels, pastes or creams, solid systems like powders, granulates, tablets, capsules, pellets, microgranulates, minitablets, microcapsules, micropellets, suppositories; etc.

Each of the above systems can be suitably formulated for normal, delayed or accelerated release, using techniques well-known in the art.

### Formulations, Dosages, and Routes of Administration

The pharmaceutical compositions described herein can be prepared according to standard techniques, as well as those techniques described herein. For instance, the pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are known in the art. See, e.g., Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York.

The therapeutic agent may be encapsulated in the lipid composition, for instance, the therapeutic agent may be completely or partially located in the interior space of the LNPs, within the lipid layer/membrane, or associated with the exterior surface of the lipid layer/membrane. One purpose of incorporating therapeutic agents into LNPs is to protect the therapeutic agents from environments which may contain enzymes or chemicals or conditions that degrade the therapeutic agents and/or systems or receptors that cause the rapid excretion of the therapeutic agents. Moreover, incorporating therapeutic agents into LNPs may promote uptake of the therapeutic agent, and hence, may enhance the therapeutic effect.

In some embodiments, in the pharmaceutical composition, the lipid components to therapeutic agent ratio (mass/mass ratio; w/w ratio) can range from about 1:1 to about 25:1, 10:1 to about 14:1, about 3:1 to about 15:1, about 4:1 to about 10:1, about 5:1 to about 9:1, or about 6:1 to about 9:1.

The lipid composition or pharmaceutical composition may contain about 5 to about 95% by weight the therapeutic agent, based on the weight of the lipid composition or pharmaceutical composition. In some embodiments, the lipid composition or pharmaceutical composition contains about 5%, about 10%, about 20, about 30, about 40, about 50, about 60, about 70, about 80, about 90, or about 95% by weight, based on the weight of the LNP or pharmaceutical composition, of the therapeutic agent. In some embodiments, the lipid composition or pharmaceutical composition contains the therapeutic agent in an amount about 5-95%, about 5-90%, about 5-80%, about 5-70%, about 5-60%, about 5-50%, about 5-40%, about 5-30%, about 5-20%, about 5-10%, about 10-95%, about 10-90%, about 10-80%, about 10-70%, about 10-60%, about 10-50%, about 10-40%, about 10-30%, about 10-20%, about 20-95%, about 20-90%, about 20-80%, about 20-70%, about 20-60%, about 20-50%, about 20-40%, about 20-30%, about 30-95%, about 30-90%, about 30-80%, about 30-70%, about 30-60%, about 30-50%, about 30-40%, about 40-95%, about 40-90%, about 40-80%, about 40-70%, about 40-60%, about 40-50%, about 50-95%, about 50-90%, about 50-80%, about 50-70%, about 50-60%, about 60-95%, about 60-90%, about 60-80%, about 60-70%, about 70-95%, about 70-90%, about 70-80%, about 80-95%, about 80-90%, or about 90-95%, based on the weight of the lipid composition or pharmaceutical composition.

The lipid composition or pharmaceutical compositions can contain total lipids at an amount of about 5 to about 95% by weight, based on the weight of the lipid composition or pharmaceutical composition. In some embodiments, the lipid composition or pharmaceutical compositions contain total lipids at an amount of about 5-95%, about 5-90%, about 5-80%, about 5-70) %, about 5-60%, about 5-50%, about 5-40%, about 5-30%, about 5-20%, about 5-10%, about 10-95%, about 10-90%, about 10-80%, about 10-70%, about 10-60%, about 10-50%, about 10-40%, about 10-30%, about 10-20%, about 20-95%, about 20-90%, about 20-80%, about 20-70%, about 20-60%, about 20-50%, about 20-40%, about 20-30%, about 30)-95%, about 30-90%, about 30-80%, about 30-70%, about 30-60%, about 30-50%, about 30)-40%, about 40-95%, about 40-90%, about 40-80%, about 40-70%, about 40-60%, about 40-50%, about 50-95%, about 50-90%, about 50-80%, about 50-70%, about 50-60%, about 60-95%, about 60-90%, about 60-80%, about 60-70%, about 70-95%, about 70-90%, about 70-20) 80%, about 80-95%, about 80-90%, or about 90-95%, based on the weight of the lipid composition or pharmaceutical composition.

The compositions of this disclosure may be administered by various routes, for example, to effect systemic delivery via intravenous, parenteral, intraperitoneal, or topical routes. In some embodiments, a siRNA may be delivered intracellularly, for example, in cells of a target tissue such as lung or liver, or in inflamed tissues. In some embodiments, this disclosure provides a method for delivery of siRNA in vivo. A nucleic acid-lipid composition may be administered intravenously, subcutaneously, or intraperitoneally to a subject.

The compositions and methods of the disclosure may be administered to subjects by a variety of mucosal administration modes, including by oral, rectal, vaginal, intranasal, intrapulmonary, or transdermal or dermal delivery, or by topical delivery to the eyes, ears, skin, or other mucosal surfaces. In some aspects of this disclosure, the mucosal tissue layer includes an epithelial cell layer. The epithelial cell can be pulmonary, tracheal, bronchial, alveolar, nasal, buccal, epidermal, or gastrointestinal. Compositions of this disclosure can be administered using conventional actuators such as mechanical spray devices, as well as pressurized, electrically activated, or other types of actuators.

Compositions of this disclosure may be administered in an aqueous solution as a nasal or pulmonary spray and may be dispensed in spray form by a variety of methods known to those skilled in the art. Pulmonary delivery of a composition of this disclosure is achieved by administering the composition in the form of drops, particles, or spray, which can be, for example, aerosolized, atomized, or nebulized. Particles of the composition, spray, or aerosol can be in either a liquid or solid form. Non-limiting examples of systems for dispensing liquids as a nasal spray are disclosed in U.S. Pat. No. 4,511,069. Such formulations may be conveniently prepared by dissolving compositions according to the present disclosure in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in TRANSDERMAL SYSTEMIC MEDICATION, Y. W. Chien ed., Elsevier Publishers, New York, 1985; and in U.S. Pat. No. 4,778,810. Additional aerosol delivery forms may include, e.g., compressed air-Jet-, ultrasonic-, and piezoelectric nebulizers, which deliver the biologically active agent dissolved or suspended in a pharmaceutical solvent, e.g., water, ethanol, or mixtures thereof.

Nasal and pulmonary spray solutions of the present disclosure typically comprise the drug or drug to be delivered, optionally formulated with a surface active agent, such as a nonionic surfactant (e.g., polysorbate-80), and one or more buffers. In some embodiments of the present disclosure, the nasal spray solution further comprises a propellant. The pH of the nasal spray solution may be from pH 6.8 to 7.2. The pharmaceutical solvents employed can also be a slightly acidic aqueous buffer of pH 4-6. Other components may be added to enhance or maintain chemical stability, including preservatives, surfactants, dispersants, or gases.

In some embodiments, this disclosure is a pharmaceutical product which includes a solution containing a composition of this disclosure and an actuator for a pulmonary, mucosal, or intranasal spray or aerosol.

A dosage form of the composition of this disclosure can be liquid, in the form of droplets or an emulsion, or in the form of an aerosol.

A dosage form of the composition of this disclosure can be solid, which can be reconstituted in a liquid prior to administration. The solid can be administered as a powder. The solid can be in the form of a capsule, tablet, or gel.

To prepare compositions for pulmonary delivery within the present disclosure, the biologically active agent can be combined with various pharmaceutically acceptable additives, as well as a base or carrier for dispersion of the active agent(s).

Examples of additives include pH control agents such as arginine, sodium hydroxide, glycine, hydrochloric acid, citric acid, and mixtures thereof. Other additives include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the composition, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-gly colic acid) copolymer, polyhydroxy butyric acid, poly(hydroxy butyric acid-glycolic acid) copolymer, and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination, and enhanced structural integrity can be imparted to the carrier by partial crystallization, ionic bonding, crosslinking, and the like. The carrier can be provided in a variety of forms, including fluid or viscous solutions, gels, pastes, powders, microspheres, and films for direct application to the nasal mucosa. The use of a selected carrier in this context may result in promotion of absorption of the biologically active agent.

Compositions for mucosal, nasal, or pulmonary delivery may contain a hydrophilic low molecular weight compound as a base or excipient. Such hydrophilic low molecular weight compounds may provide a passage medium through which a water-soluble active agent, such as a physiologically active peptide or protein, may diffuse through the base to the body surface where the active agent is absorbed. The hydrophilic low molecular weight compound may optionally absorb moisture from the mucosa or the administration atmosphere and may dissolve the water-soluble active peptide. In some embodiments, the molecular weight of the hydrophilic low molecular weight compound is less than or equal to 10,000, such as not more than 3,000. Examples of hydrophilic low molecular weight compounds include polyol compounds, such as oligo-, di- and monosaccharides including sucrose, mannitol, lactose, L-arabinose, D-erythrose, D-ribose, D-xylose, D-mannose, D-galactose, lactulose, cellobiose, gentibiose, glycerin, polyethylene glycol, and mixtures thereof. Further examples of hydrophilic low molecular weight compounds include N-methylpyrrolidone, alcohols (e.g., oligovinyl alcohol, ethanol, ethylene glycol, propylene glycol, etc.), and mixtures thereof.

The compositions of this disclosure may alternatively contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the biologically active agent may be administered in a time release formulation, for example in a composition which includes a slow release polymer. The active agent can be prepared with carriers that will protect against rapid release, for example a controlled release vehicle such as a polymer, microencapsulated delivery system, or bioadhesive gel. Prolonged delivery of the active agent, in various compositions of the disclosure can be brought about by including in the composition agents that delay absorption, for example, aluminum monosterate hydrogels and gelatin.

In some embodiments, the lipid composition, pharmaceutical compositions, or dosage units contain about 0.01 to about 1000 mg of one or more lipid compounds described herein. In some embodiments, the lipid composition, pharmaceutical compositions, or dosage units contain about 0.01, about 0.1, about 0.5, about 1, about 5, about 10, about 25, about 50, about 75, about 100, about 125, about 150, about 175, about 200, about 225, 250, about 275, about 300, about 350, about 400, about 450, about 500, about 550, about 600, about 650, about 700, about 750, about 800, about 850, about 900, about 950, or about 1000 mg of one or more lipid compounds described herein. In some embodiments, the lipid composition, pharmaceutical compositions, or dosage units contain about 0.01 to about 750 mg, about 0.01 to about 500 mg, about 0.01 to about 250 mg, about 0.01 to about 100 mg, about 0.01 to about 50 mg, about 0.01 to about 25 mg, about 0.01 to about 10 mg, about 0.01 to about 5 mg, about 0.01 to about 0.1 mg, about 0.1 to about 1000 mg, about 0.1 to about 750 mg, about 0.1 to about 500 mg, about 0.1 to about 250 mg, about 0.1 to about 100 mg, about 0.1 to about 50 mg, about 0.1 to about 25, about 0.1 to about 10 mg, about 0.1 to about 5 mg, about 0.1 to about 1 mg, about 1 to about 1000 mg, about 1 to about 750 mg, about 1 to about 500 mg, about 1 to about 250 mg, about 1 to about 100 mg, about 1 to about 50 mg, about 1 to about 25 mg, about 1 to about 10 mg, about 1 to about 5 mg, about 5 to about 1000 mg, about 5 to about 750 mg, about 5 to about 500 mg, about 5 to about 250 mg, about 5 to about 100 mg, about 5 to about 50 mg, about 5 to about 25 mg, about 5 to about 10 mg, about 10 to about 1000 mg, about 10 to about 750 mg, about 10 to about 500, about 10 to about 250 mg, about 10 to about 100 mg, about 10 to about 50 mg, about 10 to about 25 mg, about 25 to about 1000 mg, about 25 to about 750 mg, about 25 to about 500 mg, about 25 to about 250 mg, about 25 to about 100 mg, about 25 to about 50 mg, about 50 to about 1000, mg about 50 to about 750 mg, about 50 to about 500 mg, about 50 to about 250 mg, about 50 to about 100 mg, about 100 to about 1000 mg, about 100 to about 750 mg, about 100 to about 500 mg, about 100 to about 250 mg, about 250 to about 1000 mg, about 250 to about 750 mg, about 250 to about 500 mg, about 500 to about 1000 mg, about 500 to about 750 mg, or about 750 to about 1000 mg of one or more lipid compounds described herein.

### Methods of Using the Lipid Composition

Another aspect of the present disclosure provides methods for delivering a therapeutic agent to a subject (e.g., a patient) in need thereof, comprising administering to said subject (e.g., patient) the pharmaceutical composition comprises a lipid nanoparticle composition comprising the ionizable lipid compound described herein, a pharmaceutically acceptable salt thereof, and/or a stereoisomer of any of the foregoing, and the therapeutic agent.

In some embodiments, provided herein is a method of delivering therapeutic cargo to at least one organ chosen from the pancreas, one or both lungs, and the spleen of a subject in need thereof with a minimum amount delivered elsewhere in body, such as in the liver, of the subject. In some embodiments, the method delivers therapeutic cargo to the pancreas and/or one or both lungs a subject in need thereof with a minimum amount delivered elsewhere in body, such as in the liver, of the subject.

In some embodiments, less than 55%, 50%, 45%, 40%, 35%, 30%, 25%, 20%, 15%, 10%, 5% or 1% of the total therapeutic cargo administered to the subject is delivered to the liver of the subject. In some embodiments, less than 6%, 7%, 8%, 10%, 11%, 12%, 13%, 14%, 15%, 16%, 17%, 18%, 19%, or 20% of the total therapeutic cargo administered to the subject is delivered to the liver of the subject.

In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic cargo administered to the subject is delivered to the pancreas, spleen, and/or one or both lungs of the subject. In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic cargo administered to the subject is delivered to the pancreas of the subject. In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic cargo administered to the subject is delivered to the lungs of the subject. In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic cargo administered to the subject is delivered to the spleen of the subject.

In some embodiments, the total therapeutic cargo administered to the subject has a spleen to liver ratio of at least 0.5, 0.6, 0.7, 0.8, 0.9, 10, 1.1, 1.2, 1.3, 1.4, 1.5, 1.6, 1.7, 1.8, 1.9, 2, 3, 4, 5, 6, 7, 8, 9, or 10. In some embodiments, the total therapeutic cargo administered to the subject has a spleen to liver ratio of at least 1. In some embodiments, the total therapeutic cargo administered to the subject has spleen to liver ratio of at least 5.

As used herein, the percent amount of the total therapeutic cargo administered to the subject and delivered to a location in the subject is measured by the level of protein expression, or mRNA knockdown level.

In some embodiments, the method of delivering a therapeutic cargo disclosed above comprises administering to a subject a lipid nanoparticle composition comprising therapeutic cargo. In some embodiments, the lipid nanoparticles in the lipid nanoparticle composition are formed from one or more compounds chosen from ionizable lipids of Formula (I)—(VII), pharmaceutically acceptable salts thereof, and stereoisomers of any of the foregoing. In some embodiments, the lipid nanoparticles are formed from one or more compounds chosen from ionizable lipids of Formula (I), pharmaceutically acceptable salts thereof, and stereoisomers of any of the foregoing. In some embodiments, the lipid nanoparticles are formed from one or more compounds chosen from ionizable lipids of Formula (II), pharmaceutically acceptable salts thereof, and stereoisomers of any of the foregoing. In some embodiments, the lipid nanoparticles are formed from one or more compounds chosen from ionizable lipids of Formula (III), pharmaceutically acceptable salts thereof, and stereoisomers of any of the foregoing. In some embodiments, the lipid nanoparticles are formed from one or more compounds chosen from ionizable lipids of Formula (IV), pharmaceutically acceptable salts thereof, and stereoisomers of any of the foregoing. In some embodiments, the lipid nanoparticles are formed from one or more compounds chosen from ionizable lipids of Formula (V), pharmaceutically acceptable salts thereof, and stereoisomers of any of the foregoing. In some embodiments, the lipid nanoparticles are formed from one or more compounds chosen from ionizable lipids of Formula (VI), pharmaceutically acceptable salts thereof, and stereoisomers of any of the foregoing. In some embodiments, the lipid nanoparticles are formed from one or more compounds chosen from ionizable lipids of Formula (VII), pharmaceutically acceptable salts thereof, and stereoisomers of any of the foregoing.

In some embodiments, the lipid nanoparticles and lipid nanoparticle compositions disclosed herein may be used for a variety of purposes, including delivery of encapsulated or associated (e.g., complexed) therapeutic agents such as nucleic acids to cells, in vitro and/or in vivo. Accordingly, in some embodiments, provided are methods of treating or preventing diseases or disorders in a subject in need thereof comprising administering to the subject a lipid nanoparticle. In some embodiments, the lipid nanoparticle encapsulates or is associated with a suitable therapeutic agent, wherein the lipid nanoparticle comprises one or more of the novel ionizable lipids described herein, a pharmaceutically acceptable salt thereof, and/or a stereoisomer of any of the foregoing.

In some embodiments, the lipid nanoparticles of the present disclosure are useful for delivery of therapeutic cargo. In some embodiments, therapeutic cargo is chosen from one or more nucleic acids, including, e.g., mRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc. Therefore, in some embodiments, disclosed herein are methods of inducing expression of a desired protein in vitro and/or in vivo by contacting cells with a lipid nanoparticle comprising one or more novel ionizable lipids described herein, wherein the lipid nanoparticle encapsulates or is associated with a nucleic acid that is expressed to produce a desired protein (e.g., a messenger RNA or plasmid encoding the desired protein) or inhibit processes that terminate expression of mRNA (e.g., miRNA inhibitors). In some embodiments, disclosed herein are methods of decreasing expression of target genes and proteins in vitro and/or in vivo by contacting cells with a lipid nanoparticle comprising one or more novel ionizable lipids described herein, wherein the lipid nanoparticle encapsulates or is associated with a nucleic acid that reduces target gene expression (e.g., an antisense oligonucleotide or small interfering RNA (siRNA)). In some embodiments, disclosed herein are methods for co-delivery of one or more nucleic acid (e.g. mRNA and plasmid DNA), separately or in combination, such as may be useful to provide an effect requiring colocalization of different nucleic acids (e.g. mRNA encoding for a suitable gene modifying enzyme and DNA segment(s) for incorporation into the host genome).

In some embodiments, the lipid nanoparticles compositions are useful for upregulation of endogenous protein expression by delivering miRNA inhibitors targeting one specific miRNA or a group of miRNA regulating one target mRNA or several mRNA. In some embodiments, provided herein are methods for upregulating endogenous protein expression comprising delivering miRNA inhibitors targeting one or more miRNA regulating one or more mRNA.

In some embodiments, the lipid nanoparticle compositions are useful for down-regulating (e.g., silencing) the protein levels and/or mRNA levels of target genes. In some embodiments, provided herein are methods for down-regulating (e.g., silencing) protein and/or mRNA levels of target genes.

In some embodiments, the lipid nanoparticles are useful for delivery of mRNA and plasmids for expression of transgenes. In some embodiments, provided herein are methods for delivering mRNA and plasmids for expression of transgenes.

In some embodiments, the lipid nanoparticle compositions are useful for inducing a pharmacological effect resulting from expression of a protein, e.g., increased production of red blood cells through the delivery of a suitable erythropoietin mRNA, or protection against infection through delivery of mRNA encoding for a suitable antigen or antibody. In some embodiments, provided herein are methods for inducing a pharmacological effect resulting from expression of a protein, e.g., increased production of red blood cells through the delivery of a suitable erythropoietin mRNA, or protection against infection through delivery of mRNA encoding for a suitable antigen or antibody.

Non-limiting exemplary embodiments of the ionizable lipids of the present disclosure, lipid nanoparticles and compositions comprising the same, and their use to deliver agents (e.g., therapeutic agents, such as nucleic acids) and/or to modulate gene and/or protein expression are described in further detail below.

In some embodiments, the disclosure relates to a method of gene editing, comprising contacting a cell with an LNP. In some embodiments, the disclosure relates to any method of gene editing described herein, comprising cleaving DNA.

In some embodiments, the disclosure relates to a method of cleaving DNA, comprising contacting a cell with an LNP composition.

In some embodiments, the disclosure relates to any method of cleaving DNA described herein, wherein the cleaving step comprises introducing a single stranded DNA nick. In some embodiments, the disclosure relates to any method of cleaving DNA described herein, wherein the cleaving step comprises introducing a double-stranded DNA break. In some embodiments, the disclosure relates to any method of cleaving DNA described herein, wherein the LNP composition comprises a Class 2 Cas mRNA and a guide RNA nucleic acid. In some embodiments, the disclosure relates to any method of cleaving DNA described herein, further comprising introducing at least one template nucleic acid into the cell. In some embodiments, the disclosure relates to any method of cleaving DNA described herein, comprising contacting the cell with an LNP composition comprising a template nucleic acid.

In some embodiments, the disclosure relates to any a method of gene editing described herein, wherein the method comprises administering the LNP composition to an animal, for example a human. In some embodiments, the disclosure relates to any method of gene editing described herein, wherein the method comprises administering the LNP composition to a cell, such as a eukaryotic cell.

In some embodiments, the disclosure relates to any method of gene editing described herein, wherein the method comprises administering the mRNA formulated in a first LNP composition and a second LNP composition comprising one or more of an mRNA, a gRNA, a gRNA nucleic acid, and a template nucleic acid. In some embodiments, the disclosure relates to any method of gene editing described herein, wherein the first and second LNP compositions are administered simultaneously. In some embodiments, the disclosure relates to any method of gene editing described herein, wherein the first and second LNP compositions are administered sequentially.

In some embodiments, the disclosure relates to any method of gene editing described herein, wherein the method comprises administering the mRNA and the guide RNA nucleic acid formulated in a single LNP composition.

In some embodiments, the disclosure relates to any method of gene editing described herein, wherein the gene editing results in a gene knockout.

In some embodiments, the disclosure relates to any method of gene editing described herein, wherein the gene editing results in a gene correction.

In some embodiments, the disclosure relates to methods for in vivo delivery of interfering RNA to the lung of a mammalian subject.

In some embodiments, relates to methods of treating a disease or disorder in a mammalian subject. In some embodiments, these methods comprise administering a therapeutically effective amount of a composition of this disclosure to a subject having a disease or disorder associated with expression or overexpression of a gene that can be reduced, decreased, downregulated, or silenced by the composition.

### EXAMPLES

The following examples are for illustrative purposes only and are not intended to limit, in any way, the scope of the present invention.

### Example 1. Synthesis of Compound 2243

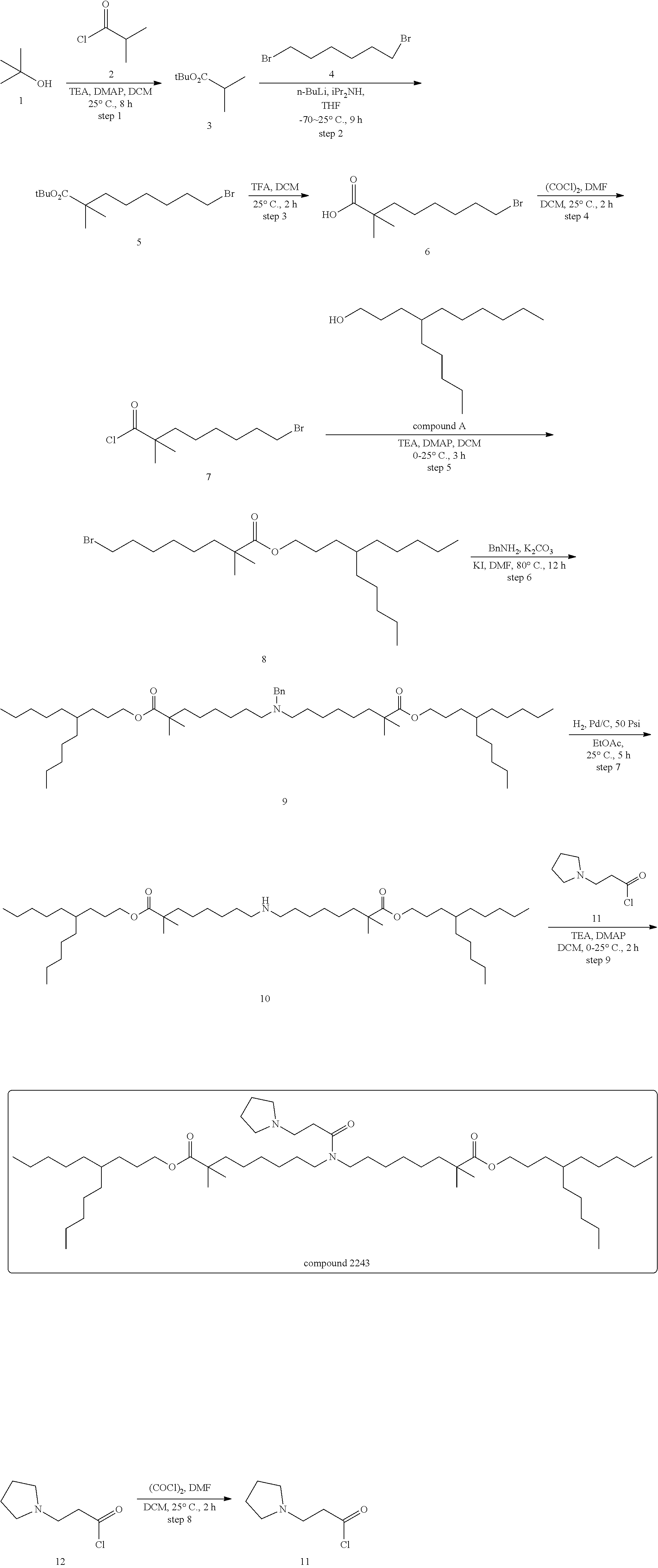

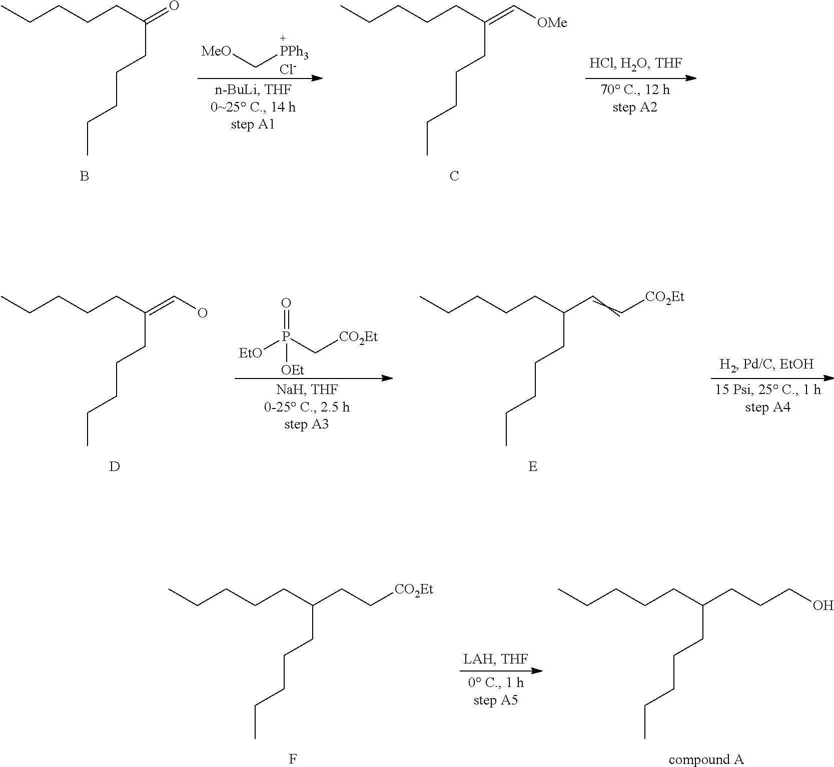

### Step A1

To a solution of methoxymethyl(triphenyl)phosphonium; chloride (24.16 g, 70.47 mmol, 3 eq.) in THF (360 mL) was added dropwise n-BμLi (2.5 M, 26.31 mL, 2.8 eq.) at 0° C. and the mixture was stirred at 25° C. for 2 hours. A solution of undecan-6-one (B) (4 g, 23.49 mmol, 1 eq.) in THF (120 mL) was added into the mixture at ° C., then stirred at 25° C. for 12 hours. The mixture was poured into H₂O (200 mL) at 0° C., and extracted with EtOAc (100 mL×3). The combined organic layer was washed with brine (100 mL×2), dried over Na₂SO₄, filtered and the filtrate was concentrated under reduced pressure to give a residue. The residue was purified by column chromatography (SiO₂, petroleum ether/ethyl acetate=100/0 to 50/1) to give 6-(methoxymethylene)undecane (C) (18 g, 90.8 mmol, 77% yield) as colorless oil.

### Step A2:

A solution of 6-(methoxymethylene)undecane (C) (18 g, 90.75 mmol, 1 eq.) in THF (72 mL) and aqeous HCl (3 M, 18.00 mL, 5.95e-1 eq.) was stirred at 70° C. for 12 hours. The mixture was poured into H₂O (100 mL) at 0° C., extracted with EtOAc (50 mL×3). The combined organic layer was washed with brine (50 mL×2), dried over Na₂SO₄, filtered and the filtrate was concentrated under reduced pressure to give a residue. The residue was purified by column chromatography (SiO₂, Petroleum ether/Ethyl acetate=100/1 to 20/1) to give 2-pentylheptanal (D) (15 g, 81.38 mmol, 90% yield) as colorless oil.

### Step A3:

To a solution of NaH (3.95 g, 98.74 mmol, 7.05 mL, 60% purity, 1.3 eq.) in THF (280 mL) was added dropwise ethyl 2-diethoxyphosphorylacetate (22.14 g, 98.74 mmol, 19.59 mL, 1.3 eq.) at 0° C., the mixture was stirred at 25° C. for 0.5 hour. A solution of 2-pentylheptanal (D) (14 g, 75.96 mmol, 1 eq.) in THF (70 mL) was added into the mixture at 0° C., then the mixture was warmed to 25° C., and stirred at 25° C. for 2 hours. The mixture was poured into H₂O (200 mL) at 0° C. extracted with EtOAc (100 mL×3). The combined organic layer was washed with brine (50 mL×2), dried over Na₂SO₄, filtered and the filtrate was concentrated under reduced pressure to get a residue. The residue was purified by column chromatography (SiO₂, Petroleum ether/Ethyl acetate=100/1 to 20/1) to give ethyl 4-pentylnon-2-enoate (E) (16 g, 62.89 mmol, 82.80% yield) as colorless oil.

### Step A4

A solution of Pd/C (2.5 g, 10% purity) and ethyl 4-pentylnon-2-enoate (E) (5 g, 19.65 mmol, 1 eq.) in EtOH (100 mL) was stirred at 25° C. for 1 hour under H₂ (15 Psi). The mixture was filtered and the filtrate was concentrated under reduced pressure to give ethyl 4-pentylnonanoate (F) (15 g, crude) as colorless oil.

### Step A5

To a solution of LAH (1.48 g, 39.00 mmol, 7.05 mL, 2 eq.) in THF (50 mL) was added a solution of ethyl 4-pentylnonanoate (F) (5 g, 19.50 mmol, 1 eq) in THF (10 mL) at 0° C. and stirred at 0° C. for 1 hour. The mixture was poured into H₂O (30 mL) at 0° C. then the mixture was filtered and the filtrate was extracted with EtOAc (50 mL*3). The combined organic layer was washed with brine (50 ml×2), dried over Na₂SO₄, filtered and the filtrate was concentrated under reduced pressure. The residue was purified by column chromatography (SiO₂, Petroleum ether/Ethyl acetate=100/1 to 20/1) to give 4-pentylnonan-1-ol (compound A) (10 g, 46.6 mmol, 80% yield) as colorless oil.

### Step 1

To a solution of 2-methylpropanoyl chloride (2) (25.50 g, 239 mmol, 25 mL, 1 eq.) in DCM (400 mL) was added a solution of 2-methylpropan-2-ol (1) (18.63 g, 251 mmol, 24 mL, 1.05 eq.) in DCM (400 mL) and then TEA (36.33 g, 359 mmol, 50 mL, 1.5 eq.) and DMAP (1.46 g, 11.97 mmol, 0.05 eq.) was added into the mixture, the mixture was stirred at 25° C. for 8 h. The mixture was added into H₂O (1000 mL), extracted with DCM (300 mL×2), organic layer was washed with brine (200 mL×2), dried over Na₂SO₄, filtered and filtrate was concentrated under reduced pressure. The crude product was distilled in vacuum (100° C., 0.08 MPa/oil pump) to give tert-butyl 2-methylpropanoate (3) (46 g, 319 mmol, 33% yield) as colorless oil.

### Step 2

To a solution of diisopropylamine (10.5 g, 104 mmol, 14.7 mL, 1.5 eq.) in THF (120 mL) was added n-BμLi (2.5 M, 41.6 mL, 1.5 eq.) at −40° C. under N₂. The mixture was stirred for 0.5 hour at −40° C. and then cooled to −70° C., the solution was added into a solution of tert-butyl 2-methylpropanoate (3) (10 g, 69.3 mmol, 1 eq.) in THF (100 mL) and stirred at −70° C., for 0.5 hour under N₂. Then a solution of 1,6-dibromohexane (4) (30.45 g, 124.82 mmol, 19.15 mL, 1.8 eq.) in THF (50 mL) was added into the mixture at −70° C., and stirred at 25° C., for 8 hours under N₂. The mixture was added into aqeuous NH₄Cl solution (200 mL) and extracted with EtOAc (200 mL×3). The combined organic phases were washed with brine (100 mL×2), dried over Na₂SO₄ and filtered and the filtrate was concentrated under reduced pressure. The residue was purified by column chromatography (SiO₂, Petroleum ether/Ethyl acetate=1/0 to 50/1) to give tert-butyl 8-bromo-2,2-dimethyl-octanoate (5) (10 g, 32.5 mmol, 47% yield) as colorless oil.

¹H NMR (400 MHz, CDCl₃), 3.41 (t, J=6.8 Hz, 2H), 1.83-1.90 (m, 2H), 1.43-1.49 (m, 14H), 1.27-1.30 (m, 6H), 1.14 (s, 6H).

### Step 3

A solution of tert-butyl 8-bromo-2,2-dimethyl-octanoate (5) (10 g, 32.55 mmol, 1 eq.) in DCM (30 mL) and TFA (46.20 g, 405.18 mmol, 30.00 mL, 12.45 eq.) was stirred at 25° C. for 2 hours. The mixture was concentrated under reduced pressure to give a residue. And then the residue was dissolved with EtOAc (200 mL), washed with NaHCO₃ (200 mL×3), brine (200 mL×2), dried over Na₂SO₄, filtered and the filtrate was concentrated under reduced pressure to give 8-bromo-2,2-dimethyl-octanoic acid (6) (6.6 g, crude) as colorless oil.

¹H NMR (400 MHz, CDCl₃), 6.34 (brs, 2H), 3.41 (t, J=6.8 Hz, 2H), 1.83-1.90 (m, 2H), 1.53-1.60 (m, 2H), 1.40-1.49 (m, 2H), 1.25-1.39 (m, 4H), 1.21 (s, 6H).

### Step 4

To a solution of 8-bromo-2,2-dimethyl-octanoic acid (6) (6.6 g, 26.3 mmol, 1 eq.) in DCM (200 mL) was added (COCl)₂ (16.7 g, 131 mmol, 11.5 mL, 5 eq.) and DMF (19.2 mg, 263 μmol, 20.2 μL, 0.01 eq.), stirred at 25° C. for 2 hours. The mixture was concentrated under reduced pressure to give 8-bromo-2,2-dimethyl-octanoyl chloride (7) (7.08 g, crude) was obtained as yellow oil.

### Step 5

To a solution of 4-pentylnonan-1-ol (compound A) (2 g, 9.33 mmol, 1 eq.), DMAP (228 mg, 1.87 mmol, 0.2 eq.) and TEA (2.83 g, 28.0 mmol, 3.90 mL, 3 eq.) in DCM (50 mL) was added a solution of 8-bromo-2,2-dimethyl-octanoyl chloride (7) (2.78 g, 10.3 mmol, 1.11 eq) in DCM (20 mL) at 0° C. stirred at 25° C. for 3 hours. The mixture was added into saturated NaHCO₃ (100 mL), extracted with EtOAc (50 mL×3), organic layer was washed with brine (100 mL×2), dried over Na₂SO₄, filtered and the filtrate was concentrated under reduced pressure. The residue was purified by column chromatography (SiO₂, Petroleum ether/Ethyl acetate=50/1 to 1/1) to give 4-pentylnonyl 8-bromo-2,2-dimethyl-octanoate (8) (3 g, 6.70 mmol, 72% yield) as colorless oil.

### Step 6

To a solution of BnNH₂ (350 mg, 3.27 mmol, 356.05 μL, 1 eq.) in DMF (30 mL) added KI (1.36 g, 8.17 mmol, 2.5 eq.) and K₂CO₃ (2.26 g, 16.33 mmol, 5 eq.), 4-pentylnonyl 8-bromo-2,2-dimethyl-octanoate (8) (3.00 g, 6.70 mmol, 2.05 eq.), then stirred at 80° C. for 12 hours. The mixture was filtered, and the filtrate was added into H₂O (50) mL), extracted with EtOAc (100 mL×3), combined organic layer was washed with brine (100 mL×2), dried over Na₂SO₄, filtered and the filtrate was concentrated under reduced pressure. The residue was purified by column chromatography (SiO₂, Petroleum ether/Ethyl acetate=100/1 to 20/1) to give 4-pentylnonyl 8-[benzyl-[7,7-dimethyl-8-oxo-8-(4-pentylnonoxy)octyl]amino]-2,2-dimethyl-octanoate (9) (1.5 g, 1.78 mmol, 55% yield) as yellow oil.

¹H NMR (400 MHz, CDCl₃), 7.22-7.25 (m, 3H), 7.10-7.15 (m, 1H), 4.07 (t, J=6.8 Hz, 2H), 3.95 (t, J=6.8 Hz, 3H), 3.45 (s, 2H), 2.30 (t, J=7.2 Hz, 3H), 1.48-1.58 (m, 9H), 1.16-1.22 (m, 7H), 1.10-1.20 (m, 62H), 1.07 (s, 10H), 0.81 (t, J=6.8 Hz, 16H).

### Step 7

To a solution of Pd/C (500 mg, 10% w/w) in EtOAc (400 mL) was added 4-pentylnonyl 8-[benzyl-[7,7-dimethyl-8-oxo-8-(4-pentylnonoxy)octyl]amino]-2,2-dimethyl-octanoate (9) (1 g, 1.19 mmol, 1 eq.), stirred at 25° C. for 5 hours under H₂ under 50 Psi. The mixture was filtered, and the filtrate was concentrated under reduced pressure. The residue was purified by column chromatography (SiO₂, Petroleum ether/Ethyl acetate=5/1 to 0.1) to give 4-pentylnonyl 8-[[7,7-dimethyl-8-oxo-8-(4-pentylnonoxy)octyl]amino]-2,2-dimethyl-octanoate (10) (500 mg, crude) as colorless oil.

### Step 8

To a solution of 3-pyrrolidin-1-ylpropanoic acid (12) (100 mg, 698 μmol, 1 eq.) in DCM (5 mL) was added (COCl)₂ (443 mg, 3.49 mmol, 305 μL, 5 eq.) and DMF (5.10 mg, 69.8 μmol, 5.3 μL, 0.1 eq.), stirred at 25° C. for 2 hours. The mixture was concentrated under reduced pressure to give compound 3-pyrrolidin-1-ylpropanoyl chloride (11) (692 mg, crude, HCl) as a yellow solid.

### Step 9

To a solution of 4-pentylnonyl 8-[[7,7-dimethyl-8-oxo-8-(4-pentylnonoxy)octyl]amino]-2,2-dimethyl-octanoate (10) (400 mg, 533.14 μmol, 1 eq.) and DMAP (13.03 mg, 106.63 μmol, 0.2 eq) in DCM (5 mL) was added TEA (269.74 mg, 2.67 mmol, 371.04 μL, 5 eq.) and 3-pyrrolidin-1-ylpropanoyl chloride (456.47 mg, 2.30 mmol, 234.93 μL, 4.32 eq., HCl) at 0° C., stirred for 2 hours at 25° C. The mixture was added into sat. NaHCO₃ (50 mL), extracted with EtOAc (20 mL×3), organic layer was washed with brine (20 mL×3), dried over Na₂SO₄, filtered and the filtrate was concentrated under reduced pressure. The residue was purified by column chromatography (SiO₂. Ethyl acetate/MeOH=50/1 to 1/1) and the product was washed with PE/ACN=1/1 (5 mL). PE phase was concentrated under reduced pressure to give compound 2243 (4-pentylnonyl 8-[[7,7-dimethyl-8-oxo-8-(4-pentylnonoxy)octyl]-(3-pyrrolidin-1-ylpropanoyl)amino]-2,2-dimethyl-octanoate) (320 mg, 362 μmol, 68% yield, 99% purity) as colorless oil.

¹H NMR (400 MHz, CDCl₃), 4.01-4.16 (m, 4H), 3.27 (t, J=7.6 Hz, 2H), 3.20 (t, J=8.0 Hz, 2H), 2.83 (brs, 2H), 2.57 (brs, 6H), 1.81 (brs, 4H), 1.48-1.58 (m, 12H), 1.20-1.35 (m, 50H), 1.16 (d, J=4.8 Hz, 12H), 0.89 (t, J=6.8 Hz, 12H). LCMS: (M+H⁺): 875.8 @ 10.888 min.

### Example 2. Synthesis of Compound 2331

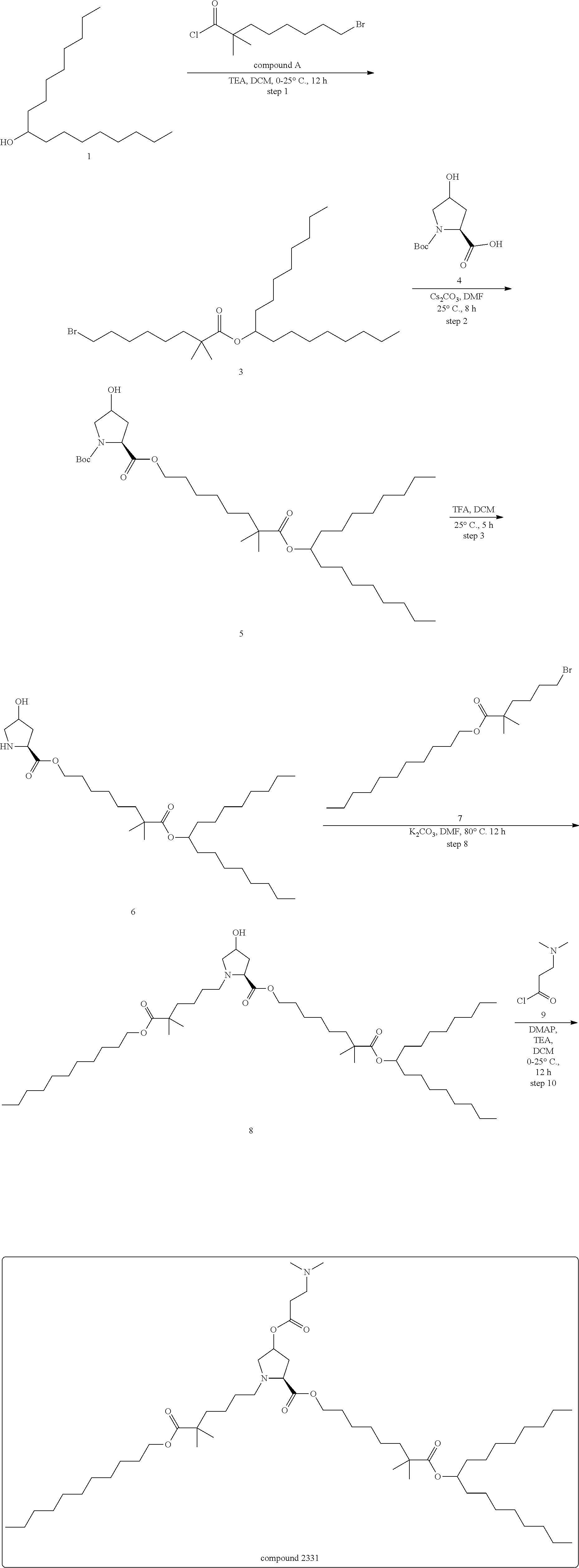

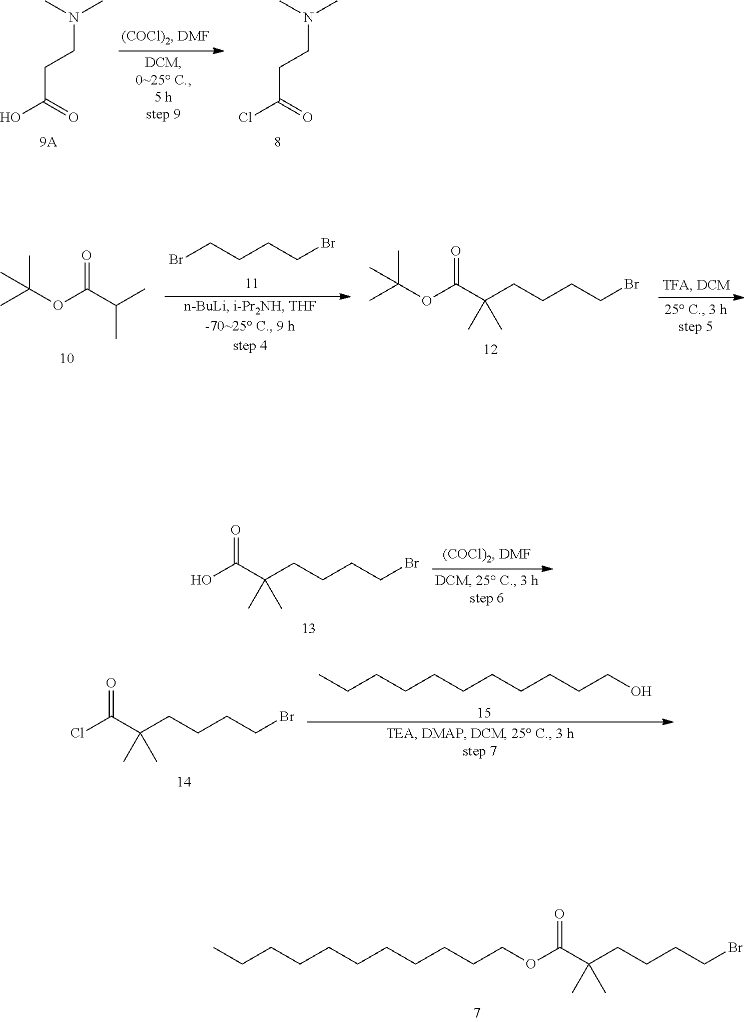

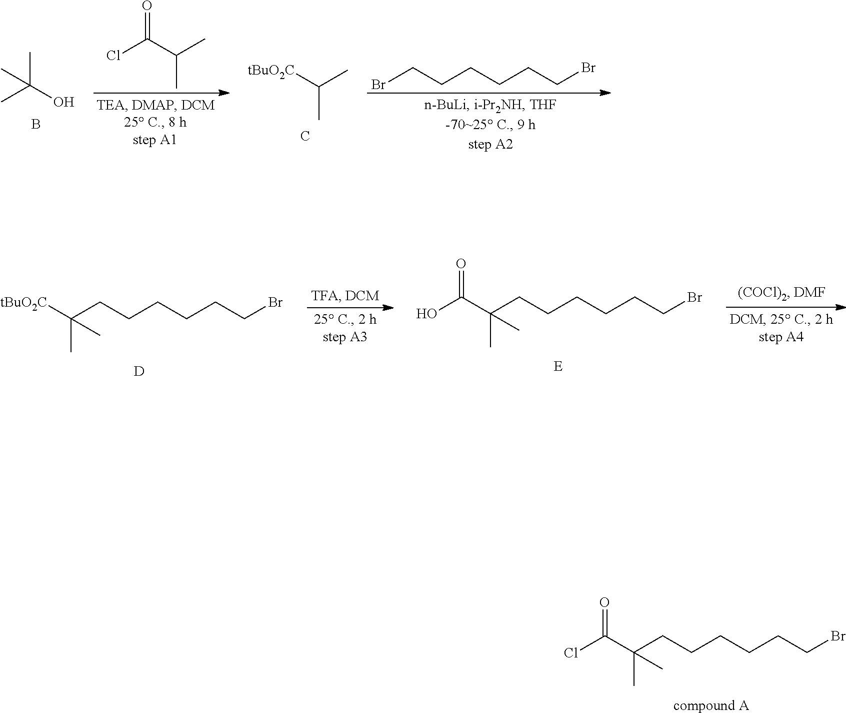

### Step A1

To a solution of 2-methylpropanoyl chloride (25.50 g, 239.32 mmol, 25 mL, 1 eq.) in DCM (400 mL) was added a solution of 2-methylpropan-2-ol (B) (18.63 g, 251.29 mmol, 24.03 mL, 1.05 eq.) in DCM (400 mL) and then TEA (36.33 g, 358.98 mmol, 49.97 mL, 1.5 eq.) and DMAP (1.46 g, 11.97 mmol, 0.05 eq.) was added into the mixture, the mixture was stirred at 25° C. for 8 hours. The mixture was added into H₂O (1000 mL), extracted with DCM (300 mL×2), organic layer was washed with brine (200 mL×2), dried over Na₂SO₄, filtered and filtrate was concentrated under reduced pressure. The crude product was distilled in vacuum (100° C., 0.08 MPa/oil pump) to give tert-butyl 2-methylpropanoate (C) (46 g, 319 mmol, 33% yield) as colorless oil.

### Step A2

To a solution of diisopropylamine (10.53 g, 104.01 mmol, 14.70 mL, 1.5 eq.) in THF (120 mL) was added n-BμLi (2.5 M, 41.61 mL, 1.5 eq.) at −40° C., under N₂. The mixture was stirred for 0.5 hours at −40° C., and then cooled to −70° C., the solution was added into a solution of tert-butyl 2-methylpropanoate (C) (10 g, 69.34 mmol, 1 eq.) in THF (100 mL) and stirred at −70° C. for 0.5 hour under N₂. Then a solution of 1,6-dibromohexane (30.45 g, 124.82 mmol, 19.15 mL, 1.8 eq) in THF (50 mL) was added into the mixture at −70° C., and stirred at 25° C. for 8 hours under N₂. The mixture was added into aq. NH₄Cl solution (200 mL) and extracted with EtOAc (200 mL×3). The combined organic phases were washed with brine (100 mL×2), dried over Na₂SO₄ and filtered and the filtrate was concentrated under reduced pressure. The residue was purified by column chromatography (SiO₂, Petroleum ether/Ethyl acetate=1/0 to 50/1) to give tert-butyl 8-bromo-2,2-dimethyl-octanoate (D) (10 g, 32.55 mmol, 46.93% yield) as colorless oil.

¹H NMR (400 MHz, CDCl₃), 3.41 (t, J=6.8 Hz, 2H), 1.83-1.90 (m, 2H), 1.43-1.49 (m, 14H), 1.27-1.30 (m, 6H), 1.14 (s, 6H).

### Step A3

A solution of tert-butyl 8-bromo-2,2-dimethyl-octanoate (D) (10 g, 32.55 mmol, 1 eq.) in DCM (30 mL) and TFA (46.20 g, 405.18 mmol, 30.00 mL, 12.45 eq.) was stirred at 25° C. for 2 hours. The mixture was concentrated under reduced pressure to give a residue. And then the residue was dissolved with EtOAc (200 mL), washed with NaHCO₃ (200 mL×3), brine (200 mL×2), dried over Na₂SO₄, filtered and the filtrate was concentrated under reduced pressure to give 8-bromo-2,2-dimethyl-octanoic acid (E) (6.6 g, crude) as colorless oil.

¹H NMR (400 MHz, CDCl₃), 6.34 (brs, 2H), 3.41 (t, J=6.8 Hz, 2H), 1.83-1.90 (m, 2H), 1.53-1.60 (m, 2H), 1.40-1.49 (m, 2H), 1.25-1.39 (m, 4H), 1.21 (s, 6H).

### Step A4

To a solution of 8-bromo-2,2-dimethyl-octanoic acid (E) (6.6 g, 26.28 mmol, 1 eq.) in DCM (200 mL) was added (COCl)₂ (16.68 g, 131.39 mmol, 11.50 mL, 5 eq.) and DMF (19.21 mg, 262.78 μmol, 20.22 μL, 0.01 eq.), stirred at 25° C. for 2 hours. The mixture was concentrated under reduced pressure to give compound A (8-bromo-2,2-dimethyl-octanoyl chloride) (7.08 g, crude) as yellow oil.

### Step 1

To a solution of heptadecan-9-ol (1) (3.96 g, 15.45 mmol, 1 eq.), 8-bromo-2,2-dimethyl-octanoyl chloride (5 g, 18.55 mmol, 1.2 eq.) in DCM (100 mL) was added TEA (6.26 g, 61.82 mmol, 8.60 mL, 4 eq.) at 0° C. The mixture was stirred at 25° C. for 12 hr. The reaction mixture was diluted with H₂O 100 mL and extracted with EtOAc 150 mL (50 mL×3). The combined organic layers were dried over Na₂SO₄, filtered and concentrated under reduced pressure to give a residue. The residue was purified by column chromatography (SiO₂, Petroleum ether/Ethyl acetate=1/0 to 1/0) to give 1-octylnonyl 8-bromo-2,2-dimethyl-octanoate (3) (4 g, 8.17 mmol, 53% yield) as yellow oil.

### Step 2

A mixture of (2S)-1-tert-butoxycarbonyl-4-hydroxy-pyrrolidine-2-carboxylic acid (4) (787.17 mg, 3.40 mmol, 1 eq.), 1-octylnonyl 8-bromo-2,2-dimethyl-octanoate (3) (2 g, 4.08 mmol, 1.2 eq), Cs₂CO₃ (2.44 g, 7.49 mmol, 2.2 eq.) in DMF (800 mL) was stirred at 25° C. for 8 hours under N₂ atmosphere. The reaction mixture was diluted with H₂O 50 ml, and extracted with EtOAc 60 mL (20 mL×3). The combined organic layers were dried over Na₂SO₄, filtered and concentrated under reduced pressure to give a residue. The residue was purified by column chromatography (SiO₂, Petroleum ether/Ethyl acetate=10/1 to 3/1) to give O1-tert-butyl O2-[7,7-dimethyl-8-(1-octylnonoxy)-8-oxo-octyl] (2S)-4-hydroxypyrrolidine-1,2-dicarboxylate (5) (2 g, 3.13 mmol, 92% yield) as a white solid.

¹H NMR (400 MHz, CDCl₃), 4.81-4.85 (m, 1H), 4.35-4.51 (m, 2H), 4.08-4.16 (m, 2H), 3.54-3.68 (m, 2H), 2.20-2.30 (m, 1H), 2.05-2.11 (m, 1H), 1.60-1.70 (m, 2H), 1.40-1.55 (m, 15H), 1.20-1.35 (m, 30H), 1.16 (s, 6H), 0.88 (t, J=6.8 Hz, 6H).

### Step 3

To a solution of O1-tert-butyl O2-[7,7-dimethyl-8-(1-octylnonoxy)-8-oxo-octyl] (2S)-4-hydroxypyrrolidine-1,2-dicarboxylate (5) (2 g, 3.13 mmol, 1 eq.) in DCM (20 mL) was added TFA (6.16 g, 54.03 mmol, 4 mL, 17.29 eq.). The mixture was stirred at 25° C. for 5 hours. The reaction mixture was adjusted pH=7 with saturated NaHCO₃ aqueous and extracted with 600 mL EtOAc (200 mL×3), dried over Na₂SO₄, filtered and concentrated under reduced pressure to give compound [7,7-dimethyl-8-(1-octylnonoxy)-8-oxo-octyl] (2S)-4-hydroxypyrrolidine-2-carboxylate (6) (1.3 g, 2.41 mmol, 77% yield) as yellow oil without purification.

### Step 4

To a solution of N-isopropylpropan-2-amine (1.05 g, 10.40 mmol, 1.47 mL, 1.5 eq.) in THF (35 mL) was added dropwise n-BμLi (2.5 M, 4.16 mL, 1.5 eq.) at −40° C. under N₂. After addition, the mixture was stirred at this temperature for 0.5 hour, and then cooled −70° C. The solution was added into a solution of tert-butyl 2-methylpropanoate (10) (1 g, 6.93 mmol, 1 eq.) in THF (50 mL) and stirred at −70° C. for 0.5 hour, then a solution of 1,4-dibromobutane (11) (2.69 g, 12.48 mmol, 1.51 mL, 1.8 eq.) in THF (10 mL) was added dropwise at −70° C. The resulting mixture was stirred at 25° C. for 8 hours. The reaction mixture was quenched by addition of 100 mL aqeuous NH₄Cl at 0° C. under N₂, and then extracted with PE 150 mL (50 mL×3). The combined organic layers were washed with brine 100 mL (50 mL×2), dried over Na₂SO₄, filtered and concentrated under reduced pressure to give a residue. The residue was purified by column chromatography (SiO₂, Petroleum ether/Ethyl acetate=1/0 to 50/1) to give tert-butyl 6-bromo-2,2-dimethyl-hexanoate (12) (9 g, 32.23 mmol, 93% yield, batch 5) as colorless oil.

¹H NMR (400 MHz, CDCl₃), 3.41 (t, J=6.8 Hz, 2H), 1.34-1.53 (m, 15H), 1.13 (s, 6H)

### Step 5

To a solution of tert-butyl 6-bromo-2,2-dimethyl-hexanoate (12) (9 g, 32.23 mmol, 1 eq.) in DCM (80 mL) was added TFA (50.40 g, 442.02 mmol, 32.73 mL, 13.71 eq.). The mixture was stirred at 25° C. for 3 hours. The reaction mixture was quenched by addition of 100 mL aqeuous NaHCO₃ at 25° C., and then extracted with EtOAc 300 mL (100 mL×3). The combined organic layers were washed with brine 200 mL (100 mL×2), dried over Na₂SO₄, filtered and concentrated under reduced pressure to give 6-bromo-2,2-dimethyl-hexanoic acid (13) (5 g, 22.41 mmol, 70% yield) as colorless oil.

### Step 6

To a solution of 6-bromo-2,2-dimethyl-hexanoic acid (13) (1 g, 4.48 mmol, 1 eq.) in DCM (50 mL) was added (COCl)₂ (2.84 g, 22.41 mmol, 1.96 mL, 5 eq.) and DMF (32.76 mg, 448.22 μmol, 34.49 μL, 0.1 eq.). The mixture was stirred at 25° C. for 3 hours. The reaction mixture was concentrated under reduced pressure to give compound 6-bromo-2,2-dimethyl-hexanoyl chloride (14) (6.23 g, crude) as a colorless oil.

### Step 7:

To the suspension of undecan-1-ol (15) (0.8 g, 4.64 mmol, 1 eq.), TEA (939.63 mg, 9.29 mmol, 1.29 mL, 2 eq) and DMAP (283.61 mg, 2.32 mmol, 0.5 eq.) in DCM (50 mL) was added dropwise 6-bromo-2,2-dimethyl-hexanoyl chloride (14) (1.55 g, 5.57 mmol, 1.2 eq., HCl) in DCM (30 mL) at 25° C. The mixture was stirred at 25° C. for 3 hours under N₂ atmosphere. The reaction mixture was quenched by addition of 100 mL aqeuous NaHCO₃ at 25° C., and then extracted with 300 mL EtOAc (100 mL×3). The combined organic layers were washed with 200 mL brine (100 mL×2), dried over Na₂SO₄, filtered and concentrated under reduced pressure to give a residue. The residue was purified by column chromatography (SiO₂, Petroleum ether/Ethyl acetate=1/0 to 10/1) to give undecyl 6-bromo-2,2-dimethyl-hexanoate (7) (5 g, 13.25 mmol, 71% yield) was obtained as a colorless oil.

¹H NMR (400 MHz, CDCl₃), 4.06 (t, J=6.4 Hz, 2H), 3.40 (t, J=6.8 Hz, 2H), 1.50-1.67 (m, 6H), 1.22-1.44 (m, 18H), 1.18 (s, 6H), 0.89 (t, J=7.2 Hz, 3H)

### Step 8

To a solution of [7,7-dimethyl-8-(1-octylnonoxy)-8-oxo-octyl] (2S)-4-hydroxypyrrolidine-2-carboxylate (6) (1 g, 1.85 mmol, 1 eq.), undecyl 6-bromo-2,2-dimethyl-hexanoate (7) (838.93 mg, 2.22 mmol, 1.2 eq.) in DMF (300 mL) was added K₂CO₃ (768.08 mg, 5.56 mmol, 3 eq.). The mixture was stirred at 80° C. for 12 hours. The reaction mixture was diluted with H₂O 500 mL and extracted with EtOAc 1200 mL (400 mL×3). The combined organic layers were dried over Na₂SO₄, filtered and concentrated under reduced pressure to give a residue. The residue was purified by column chromatography (SiO₂, Petroleum ether/Ethyl acetate/NH₃·H₂O=10/1/1 to 1/1/0.5) to give [7,7-dimethyl-8-(1-octylnonoxy)-8-oxo-octyl] (2S)-1-(5,5-dimethyl-6-oxo-6-undecoxy-hexyl)-4-hydroxy-pyrrolidine-2-carboxylate (8) (1 g, 1.12 mmol, 61% yield, 94% purity) as yellow oil.

¹H NMR (400 MHz, CDCl₃), 4.81-4.85 (m, 1H), 4.48 (brs, 1H), 4.02-4.16 (m, 5H), 3.40-3.60 (m, 2H), 2.72 (brs, 1H), 2.51 (brs, 2H), 2.05-2.30 (m, 2H), 1.55-1.70 (m, 8H), 1.40-1.55 (m, 6H), 1.20-1.39 (m, 48H), 1.16 (s, 12H), 0.86-0.90 (m, 11H).

### Step 9

To a solution of 3-(dimethylamino) propanoic acid (9A) (0.5 g, 3.26 mmol, 1 eq., HCl) and oxalyl dichloride (1.24 g, 9.77 mmol, 854.82 μL, 3 eq.) in DCM (10 mL) was added two drops of DMF at 0° C. The mixture was degassed and purged with N₂ for 3 times, and stirred at 25° C. for 5 hours under N₂ atmosphere. The reaction mixture was concentrated under reduced pressure to get 3-(dimethylamino)propanoyl chloride (9) (0.5 g, crude, HCl) as yellow oil.

### Step 10

To a solution of [7,7-dimethyl-8-(1-octylnonoxy)-8-oxo-octyl] (2S)-1-(5,5-dimethyl-6-oxo-6-undecoxy-hexyl)-4-hydroxy-pyrrolidine-2-carboxylate (8) (0.5 g, 597.86 μmol, 1 eq.), 3-(dimethylamino)propanoyl chloride (9) (411.45 mg, 2.39 mmol, 4 eq., HCl) in DCM (3 mL) was added TEA (544.48 mg, 5.38 mmol, 748.94 μL, 9 eq.) at 0° C. The mixture was stirred at 25° C. for 12 hours. The reaction mixture was diluted with 50 mL H₂O and extracted with 150 mL EtOAc (50 mL×3). The combined organic layers were dried over Na₂SO₄, filtered and concentrated under reduced pressure to give a residue. The residue was purified by column chromatography (SiO₂, Petroleum ether/Ethyl acetate/NH₃·H₂O=1/0/0.1 to 3/1/0.1) and prep-HPLC (column: Phenomenex Luna C18 100×30 mm×5 um; mobile phase: [water (HCl)-ACN]; B %: 50%-80%, 10 min) to give a solution. The solution was added into saturated NaHCO₃ (100 mL), extracted with EtOAc (20 mL×3), the organic layer was washed brine (20 mL×2), dried over Na₂SO₄, filtered and the filtrate was concentrated under reduced pressure to give compound 2331 ([7,7-dimethyl-8-(1-octylnonoxy)-8-oxo-octyl] (2S)-4-[3-(dimethylamino)propanoyloxy]-1-(5,5-dimethyl-6-oxo-6-undecoxy-hexyl) pyrrolidine-2-carboxy late) (60 mg, 62.9 μmol, 11% yield, 98% purity) as yellow oil.

¹H NMR (400 MHz, CDCl₃), 5.23-5.28 (m, 1H), 4.80-4.87 (m, 1H), 4.02-4.13 (m, 4H), 3.43-3.54 (m, 2H), 2.11-2.65 (m, 14H), 1.43-1.65 (m, 13H), 1.20-1.35 (m, 50H), 1.15 (s, 12H), 0.86-0.90 (m, 9H). MS (M+H⁺): 935.7.

### Example 3. Synthesis of Compound 2333

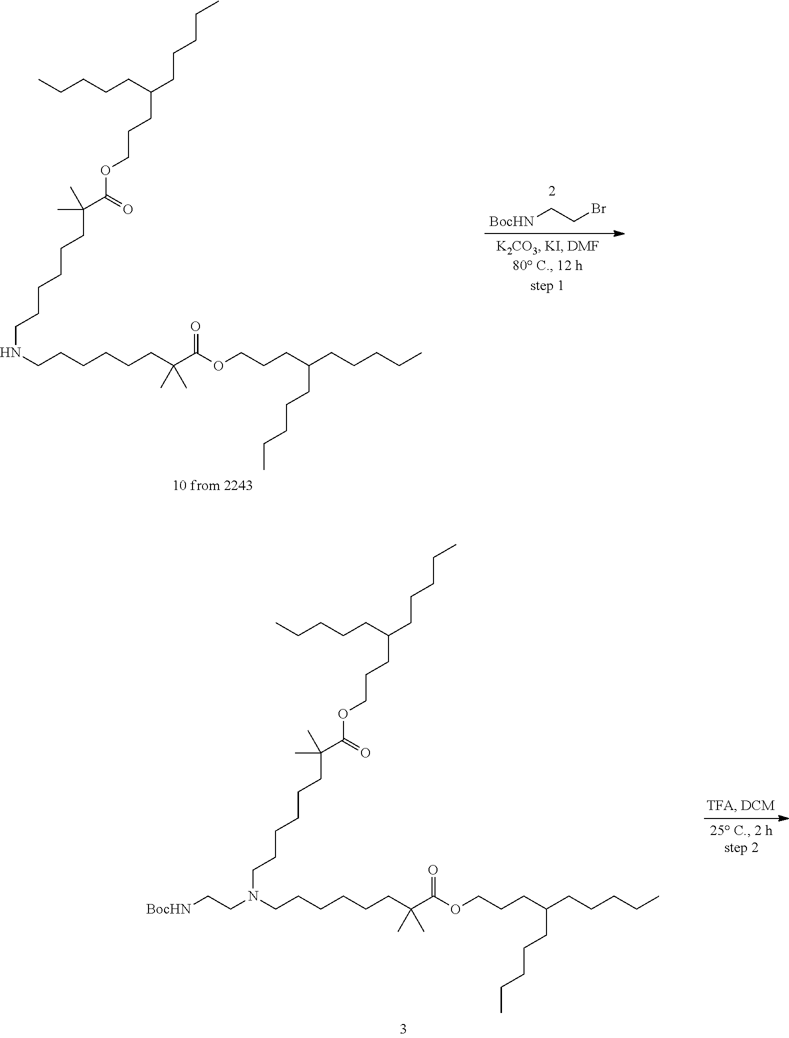 [SMILES: CCCCCC(CCCCC)CCCOC(=O)C(C)(C)CCCCCCNCCCCCCC(C)(C)C(=O)OCCCC(CCCCC)CCCCC ; CC(C)(C)OC(=O)NCCBr ; CCCCCC(CCCCC)CCCOC(=O)C(C)(C)CCCCCCN(CCCCCCC(C)(C)C(=O)OCCCC(CCCCC)CCCCC)CCNC(=O)OC(C)(C)C]

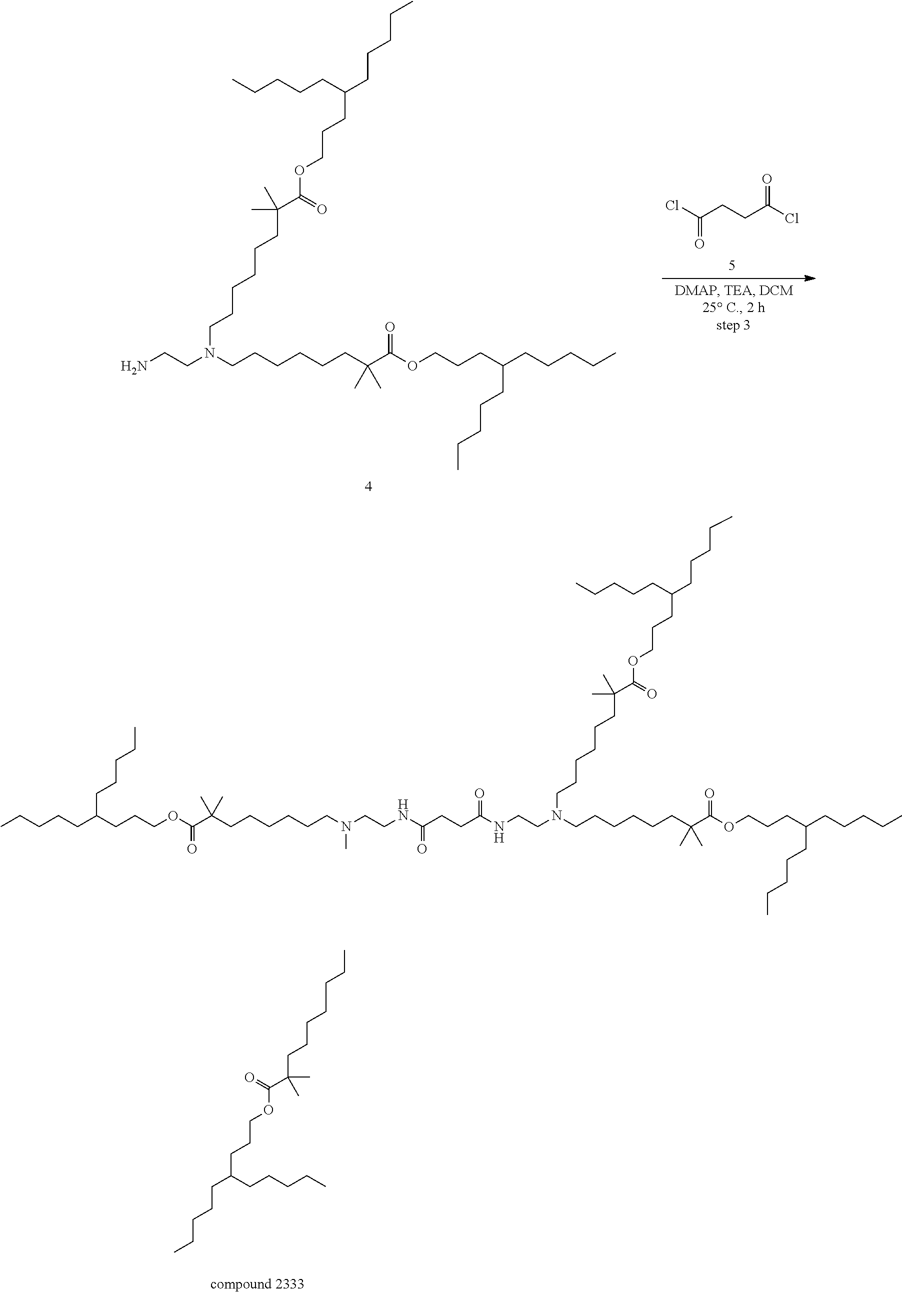

### Step 1

To a solution of 4-pentylnonyl 8-[[7,7-dimethyl-8-oxo-8-(4-pentylnonoxy)octyl]amino]-2,2-dimethyl-octanoate (10 from 2243) (500 mg, 666.43 μmol, 1 eq.) in DMF (10 mL) was added K₂CO₃ (460.54 mg, 3.33 mmol, 5 eq.) and KI (110.63 mg, 666.43 μmol, 1 eq.), then tert-butyl N-(2-bromoethyl) carbamate (2) (1.05 g, 4.66 mmol, 7 eq) was added into the mixture. The mixture was stirred at 80° C. for 12 hours. The mixture was filtered and the filtrate was added into H₂O (10 mL), extracted with EtOAc (5 mL×3), organic layer was washed with brine (5 mL×2), dried over Na₂SO₄, filtered and the filtrate was concentrated under reduced pressure. The residue was purified by column chromatography (SiO₂, Petroleum ether/Ethyl acetate=10/1 to 0/1) to give 4-pentylnonyl 8-[2-(tert-butoxy carbonylamino)ethyl-[7,7-dimethyl-8-oxo-8-(4-pentylnonoxy)octyl]amino]-2,2-dimethyl-octanoate (3) (480 mg, 537 μmol, 81% yield) was obtained as colorless oil.

### Step 2

A solution of 4-pentylnonyl 8-[2-(tert-butoxycarbonylamino)ethyl-[7,7-dimethyl-8-oxo-8-(4-pentylnonoxy)octyl]amino]-2,2-dimethyl-octanoate (3) (480 mg, 537.24 μmol, 1 eq.) in DCM (5 mL) and TFA (3.85 g, 33.77 mmol, 2.5 mL, 62.85 eq.) was stirred at 25° C. for 2 hours. The mixture was concentrated under reduced pressure to give a residue. The residue was dissolved with EtOAc (10 mL), washed with sat. NaHCO₃ (10 mL×3), brine (10 mL×2), dried over Na₂SO₄, filtered and the filtrate was concentrated under reduced pressure to give compound 4-pentylnonyl 8-[2-aminoethyl-[7,7-dimethyl-8-oxo-8-(4-pentylnonoxy)octyl]amino]-2,2-dimethyl-octanoate (4) (350 mg, crude) as colorless oil.

### Step 3

To a solution of 4-pentylnonyl 8-[2-aminoethyl-[7,7-dimethyl-8-oxo-8-(4-pentylnonoxy)octyl]amino]-2,2-dimethyl-octanoate (4) (150 mg, 189.07 μmol, 2 eq.), TEA (38.26 mg, 378.15 μmol, 52.63 μL, 4 eq.) in DCM (5 mL) was added DMAP (2.31 mg, 18.91 μmol, 0.2 eq.) and butanedioyl dichloride (15.38 mg, 99.26 μmol, 10.91 μL, 1.05 eq.) under N₂, then the mixture was stirred at 25° C. for 2 hours. The mixture was added into sat. NaHCO₃ (20 mL), extracted with EtOAc (10 mL×3), organic layer was washed with brine (10 mL×2), dried over Na₂SO₄, filtered and the filtrate was concentrated under reduced pressure. The residue was purified by column chromatography (SiO₂, Petroleum ether/Ethyl acetate=10/1 to 0/1) to get product. The product was washed with PE/ACN=1/1 (10 mL), PE phase was concentrated under reduced pressure to get the product. The product was purified by prep-TLC (SiO₂, Ethyl acetate:MeOH=10:1, added 0.3% NH₃·H₂O) and (SiO₂, Petroleum ether/Ethyl acetate=0/1, added 0.3% NH₃·H₂O) to give compound 2331 (4-pentylnonyl 8-[2-[[4-[2-[bis[7,7-dimethyl-8-oxo-8-(4-pentylnonoxy)octyl]amino]ethylamino]-4-oxo-butanoyl]amino]ethyl-[7,7-dimethyl-8-oxo-8-(4-pentylnonoxy)octyl]amino]-2,2-dimethyl-octanoate) (25 mg, 15 μmol, 83% yield, 99% purity) as colorless oil.

¹H NMR (400 MHz, CDCl₃), 6.30 (brs, 2H), 4.04 (1, J=6.4 Hz, 8H), 3.28 (brs, 4H), 2.25-2.65 (m, 16H), 1.59-1.63 (m, 10H), 1.45-1.55 (m, 8H), 1.35-1.45 (m, 6H), 1.20-1.30 (m, 100H), 1.16 (s, 24H), 0.89 (t, J=6.8 Hz, 24H). MS (M/2+H⁺): 835.0.

### Example 4. Synthesis of Compound 2335

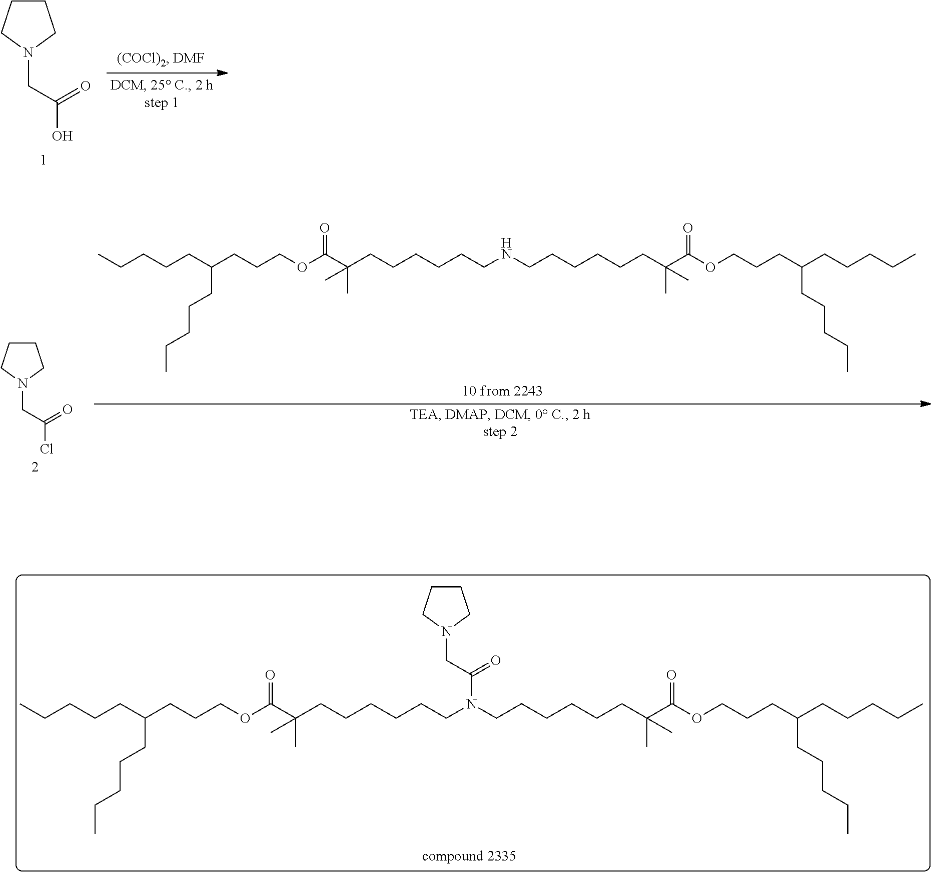

### Step 1

To a solution of 2-pyrrolidin-1-ylacetic acid (1) (100 mg, 774.25 μmol, 1 eq.) in DCM (5 mL) was added (COCl)₂ (491.38 mg, 3.87 mmol, 338.88 μL, 5 eq.) and DMF (5.66 mg, 77.43 μmol, 5.96 μL, 0.1 eq.), stirred at 25° C. for 2 hours. The mixture was concentrated under reduced pressure to give compound 2-pyrrolidin-1-ylacetyl chloride (2) (712.5 mg, crude, HCl) as a yellow solid.

### Step 2

To a solution of 4-pentylnonyl 8-[[7,7-dimethyl-8-oxo-8-(4-pentylnonoxy)octyl]amino]-2,2-dimethyl-octanoate (10 from 2243) (500 mg, 666.43 μmol, 1 eq.) and DMAP (16.28 mg, 133.29 μmol, 0.2 eq.) in DCM (5 mL) was added TEA (337.18 mg, 3.33 mmol, 463.80 μL, 5 eq.) and 2-pyrrolidin-1-ylacetyl chloride (2) (530.19 mg, 2.88 mmol, 234.93 μL, 4.32 eq., HCl) at 0° C., stirred for 2 hours at 0° C. The mixture was added into Sat. NaHCO₃ (20 mL), extracted with EtOAc (10 mL×3), organic layer was washed with brine (10 mL), dried over Na₂SO₄, filtered and the filtrate was concentrated under reduced pressure. The residue was purified by column chromatography (SiO₂, Ethyl acetate/MeOH=50/1 to 1/1) to give the product. The product was washed with PE/ACN=1/1 (5 mL), PE phase was concentrated under reduced pressure to compound 2335 (4-pentylnonyl 8-[[7,7-dimethyl-8-oxo-8-(4-pentylnonoxy)octyl]-(2-pyrrolidin-1-ylacetyl)amino]-2,2-dimethyl-octanoate) (100 mg, 115 μmol, 17% yield, 99% purity) as colorless oil.

¹H NMR (400 MHz, CDCl₃), 4.01-4.06 (m, 4H), 3.25-3.32 (m, 6H), 2.65 (brs, 2H), 1.81 (brs, 4H), 1.47-1.58 (m, 12H), 1.20-1.35 (m, 50H), 1.16 (d, J=4.8 Hz, 12H), 0.89 (t, J=6.8 Hz, 12H).

MS (M+H⁺): 861.8.

### Example 5. Synthesis of 2365

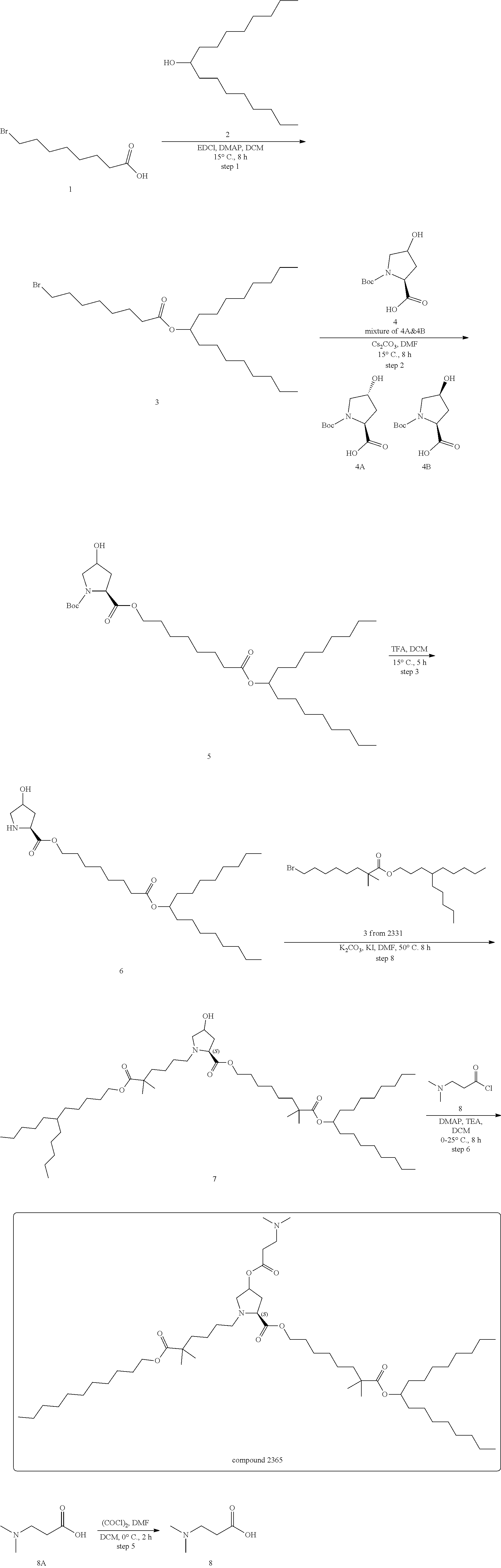

### Step 1

To a solution of 8-bromooctanoic acid (1) (4.35 g, 19.50 mmol, 1 eq) and heptadecan-9-ol (2) (5 g, 19.50 mmol, 1 eq.) in DCM (100 mL) was added EDCl (4.48 g, 23.39 mmol, 1.2 eq.) and DMAP (1.19 g, 9.75 mmol, 0.5 eq.). The mixture was stirred at 15° C. for 8 hours. The reaction mixture was quenched by addition H₂O 200 mL at 15° C., and then extracted with EtOAc 600 mL (200 mL×3). The combined organic layers were washed with brine 400 mL (200 mL×2), dried over Na₂SO₄, filtered and concentrated under reduced pressure to give a residue. The residue was purified by column chromatography (SiO₂, Petroleum ether/Ethyl acetate=1/0 to 20/1) to give 1-octylnonyl 8-bromooctanoate (3) (35 g, 75.8 mmol, 97% yield) as colorless oil.

¹H NMR (400 MHz, CDCl₃), 4.84-4.90 (m, 1H), 3.41 (t, J=6.8 Hz, 2H), 2.29 (t, J=7.6 Hz, 2H), 1.82-1.88 (m, 2H), 1.62-1.65 (m, 2H), 1.42-1.52 (m, 6H), 1.25-1.36 (m, 28H), 0.89 (t, J=7.2 Hz, 6H)

### Step 2

A mixture of 1-octylnonyl 8-bromooctanoate (3) (1 g, 2.17 mmol, 1.2 eq), (2S)-1-tert-butoxy carbonyl-4-hydroxy-pyrrolidine-2-carboxylic acid (4) (417.51 mg, 1.81 mmol, 1 eq.), Cs₂CO₃ (1.29 g, 3.97 mmol, 2.2 eq.) in DMF (10 mL) was degassed and purged with N₂ for 3 times, and then the mixture was stirred at 15° C. for 8 hours under N₂ atmosphere. The reaction mixture was quenched by addition of 50 mL H₂O at 15° C., and then extracted with EtOAc 150 mL (50 mL×3). The combined organic layers were washed with brine 100 ml (50 mL×2), dried over Na₂SO₄, filtered and concentrated under reduced pressure to give a residue. The residue was purified by column chromatography (SiO₂, Petroleum ether/Ethyl acetate=20/1 to 3/1) to give O1-tert-butyl O2-18-(1-octylnonoxy)-8-oxo-octyl] (2S)-4-hydroxypyrrolidine-1,2-dicarboxylate (5) (4.55 g, 7.44 mmol, 82% yield) as colorless oil.

¹H NMR (400 MHz, CDCl₃), 4.84-4.90 (m, 1H), 4.18-4.52 (m, 3H), 4.06-4.10 (m, 1H), 3.42-3.72 (m, 2H), 2.21-2.39 (m, 3H), 2.07-2.11 (m, 1H), 1.25-1.67 (m, 48H), 0.88 (t, J=6.8 Hz, 6H)

### Step 3

To a solution of O1-tert-butyl O2-[8-(1-octylnonoxy)-8-oxo-octyl] (2S)-4-hydroxypyrrolidine-1,2-dicarboxylate (5) (4.5 g, 7.35 mmol, 1 eq) in DCM (30 mL) was added TFA (23.10 g, 202.59 mmol, 15 mL, 27.55 eq.). The mixture was stirred at 15° C. for 3 hours. The reaction mixture was quenched by addition of aqeuous 60 mL NaHCO₃ at 15° C., and then extracted with EtOAc 150 mL (50 mL×3). The combined organic layers were washed with brine 100 mL (50 mL×2), dried over Na₂SO₄, filtered and concentrated under reduced pressure to give [8-(1-octylnonoxy)-8-oxo-octyl] (2S)-4-hydroxypyrrolidine-2-carboxylate (6) (3.76 g, 7.35 mmol, quantitative yield) as colorless oil.

### Step 4

To a solution of [8-(1-octylnonoxy)-8-oxo-octyl] (2S)-4-hydroxypyrrolidine-2-carboxylate (6) (1 g, 1.95 mmol, 1 eq) in DMF (10 mL) was added K₂CO₃ (810.16 mg, 5.86 mmol, 3 eq.) and KI (162.18 mg, 976.99 μmol, 0.5 eq.) and 4-pentylnonyl 8-bromo-2,2-dimethyl-octanoate (3 from 2331) (1.31 g, 2.93 mmol, 1.5 eq.). The mixture was stirred at 50° C. for 8 hours. The reaction mixture was quenched by addition H₂O 100 mL at 0° C., and then extracted with EtOAc 300 mL (100 mL×3). The combined organic layers were dried over Na₂SO₄, filtered and concentrated under reduced pressure to give a residue. The residue was purified by column chromatography (SiO₂, Petroleum ether/Ethyl acetate=1/0 to 3/1) to give [8-(1-octylnonoxy)-8-oxo-octyl] (2S)-1-[7,7-dimethyl-8-oxo-8-(4-pentylnonoxy)octyl]-4-hydroxy-pyrrolidine-2-carboxylate (7) (1 g, 1.14 mmol, 58% yield) as colorless oil.

### Step 5

To a solution of 3-(dimethylamino) propanoic acid (8A) (500 mg, 3.26 mmol, 1 eq., HCl) in DCM (20 mL) was added DMF (11.90 mg, 162.75 μmol, 12.52 μL, 0.05 eq.) and oxalyl dichloride (495.78 mg, 3.91 mmol, 341.92 μL, 1.2 eq.). The mixture was stirred at 0° C. for 2 hours. The mixture was concentrated under reduced pressure to give 3-(dimethylamino)propanoyl chloride (8) (560 mg, crude, HCl) as yellow oil.

### Step 6

To a solution of [8-(1-octylnonoxy)-8-oxo-octyl] (2S)-1-[7,7-dimethyl-8-oxo-8-(4-pentylnonoxy)octyl]-4-hydroxy-pyrrolidine-2-carboxylate (500 mg, 569 μmol, 1 eq.) in DCM (10 mL) was added TEA (576 mg, 5.69 mmol, 792 μL, 10 eq.) and DMAP (34.77 mg, 285 μmol, 0.5 eq.) and 3-(dimethylamino)propanoyl chloride (489 mg, 2.85 mmol, 5 eq., HCl) at 0° C. The mixture was stirred at 25° C. for 8 hours. The reaction mixture was quenched by addition of 20 mL H₂O at 0° C., and then extracted with EtOAc 30 mL (10 mL×3). The combined organic layers were dried over Na₂SO₄, filtered and concentrated under reduced pressure to give a residue. The residue was purified by column chromatography (SiO₂, Petroleum ether/Ethyl acetate=1/0 to 0/1, added 0.1% NH₃·H₂O). Then the mixture was purified by prep-HPLC (column: Phenomenex Luna C18 100×30 mm×5 μm; mobile phase: [water (HCl)-ACN]; B %: 60%-90%, 10 min), then work by sat. NaHCO₃ (100 mL) and extracted with 30 mL EtOAc (10 mL×3). The combined organic layers were dried over Na₂SO₄, filtered and concentrated under reduced pressure. Then it was purified by p-TLC (SiO₂, Petroleum ether/Ethyl acetate=0/1, added 0.1% NH₃·H₂O) to give compound 2365 ([8-(1-octylnonoxy)-8-oxo-octyl] (2S)-4-[3-(dimethylamino)propanoyloxy]-1-[7,7-dimethyl-8-oxo-8-(4-pentylnonoxy)octyl]pyrrolidine-2-carboxylate) (73 mg, 128.90 μmol, 23% yield, 100% purity) as yellow oil.

¹H NMR (400 MHz, CDCl₃), 5.20-5.27 (m, 1H), 4.85-4.88 (m, 1H), 4.01-4.12 (m, 4H), 3.11-3.54 (m, 2H), 2.26-2.62 (m, 17H), 1.59-1.60 (m, 5H), 1.47-1.51 (m, 8H), 1.24-1.33 (m, 56H), 1.15 (s, 6H), 0.89 (t, J=6.8 Hz, 12H). (M+H⁺): 977.8. MS: (M+H⁺): 977.8.

### Example 6. Synthesis of 2366

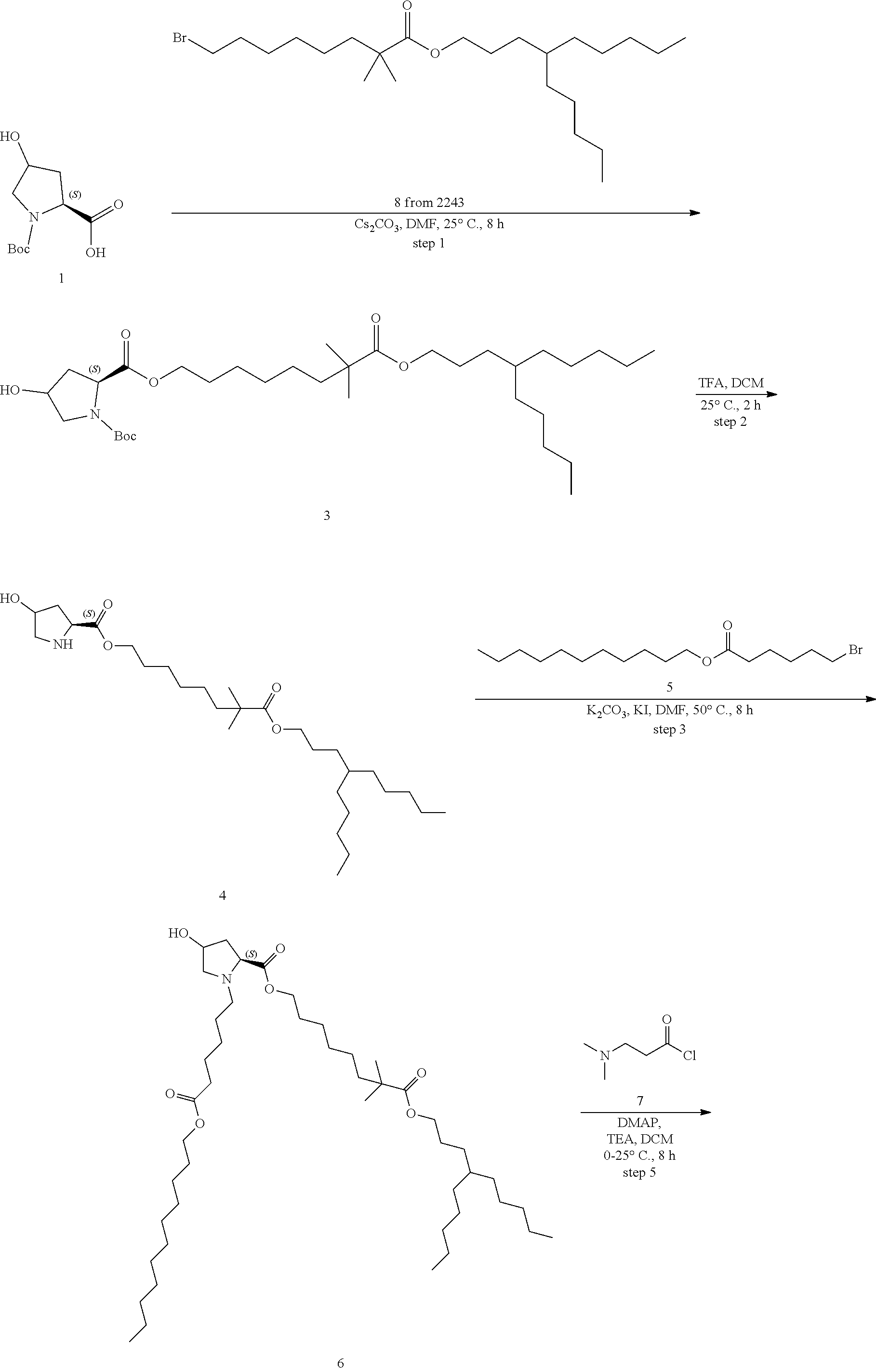

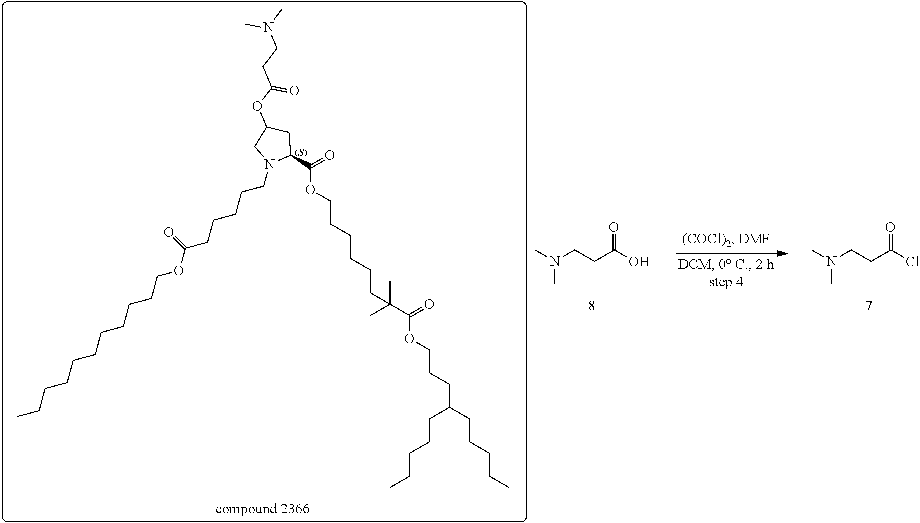

### Step 1

To a solution of 4-pentylnonyl 8-bromo-2,2-dimethyl-octanoate (8 from 2243) (2 g, 4.47 mmol, 1.2 eq.) in DMF (10 mL) was added Cs₂CO₃ (1.82 g, 5.59 mmol, 1.5 eq.) and (2S)-1-tert-butoxycarbonyl-4-hydroxy-pyrrolidine-2-carboxylic acid (1) (861 mg, 3.72 mmol, 1 eq.). The mixture was stirred at 25° C. for 8 hours. The reaction mixture was quenched by addition of 100 mL H₂O at 0° C., and then extracted with EtOAc 300 mL (100 mL×3). The combined organic layers were dried over Na₂SO₄, filtered and concentrated under reduced pressure to give a residue. The residue was purified by column chromatography (SiO₂, Petroleum ether/Ethyl acetate=1/0 to 2/1) to give O1-tert-butyl O2-[7,7-dimethyl-8-oxo-8-(4-pentylnonoxy)octyl] (2S)-4-hydroxypyrrolidine-1,2-dicarboxy late (3) (1.5 g, 2.51 mmol, 67% yield) as colorless oil.

### Step 2

To a solution of O1-tert-butyl O2-[7,7-dimethyl-8-oxo-8-(4-pentylnonoxy)octyl] (2S)-4-hydroxypyrrolidine-1,2-dicarboxylate (3) (1.5 g, 2.51 mmol, 1 eq.) in DCM (20 mL) was added TFA (10 mL). The mixture was stirred at 25° C. for 2 hours. The mixture was concentrated under reduced pressure, then adjust pH to 8 with sat. NaHCO₃, extracted with EtOAc 300 mL (100 mL×3). The combined organic layers were dried over Na₂SO₄, filtered and concentrated under reduced pressure to give a residue. The residue was purified by column chromatography (SiO₂, Petroleum ether/Ethyl acetate=3/1 to EtOAc/MeOH=5/1, added 0.1% NH₃·H₂O) to give [7,7-dimethyl-8-oxo-8-(4-pentylnonoxy)octyl] (2S)-4-hydroxypyrrolidine-2-carboxylate (4) (1 g, 2.01 mmol, 80% yield) as yellow oil.

### Step 3

To a solution of [7,7-dimethyl-8-oxo-8-(4-pentylnonoxy)octyl] (2S)-4-hydroxypyrrolidine-2-carboxylate (4) (1 g, 2.0 mmol, 1 eq.) in DMF (10 mL) was added K₂CO₃ (832.99 mg, 6.03 mmol, 3 eq.) and KI (167 mg, 1.0 mmol, 0.5 eq.) and undecyl 6-bromohexanoate (5) (1.05 g, 3.0 mmol, 1.5 eq.). The mixture was stirred at 50° C. for 8 hours. The reaction mixture was quenched by addition of 20 mL H₂O at 0° C., and then extracted with EtOAc 60 mL (20 mL×3). The combined organic layers were dried over Na₂SO₄, filtered and concentrated under reduced pressure to give a residue. The residue was purified by column chromatography (SiO₂, Petroleum ether/Ethyl acetate=1/0 to 3/1) to give [7,7-dimethyl-8-oxo-8-(4-pentylnonoxy)octyl] (2S)-4-hydroxy-1-(6-oxo-6-undecoxy-hexyl)pyrrolidine-2-carboxylate (6) (1 g, 1.31 mmol, 64.96% yield) as yellow oil.

¹H NMR (400 MHz, CDCl₃), 4.20-4.47 (m, 1H), 4.00-4.58 (m, 4H), 3.20-3.64 (m, 2H), 2.28-2.46 (m, 7H), 1.85-1.95 (m, 1H), 1.45-1.62 (m, 12H), 1.20-1.29 (m, 44H), 1.14 (s, 6H), 0.87 (t, J=6.8 Hz, 9H).

### Step 4

To a solution of 3-(dimethylamino) propanoic acid (8) (0.5 g, 3.26 mmol, 1 eq., HCl) in DCM (20 mL) was added DMF (11.90 mg, 162.75 μmol, 12.52 μL, 0.05 eq.) and oxalyl dichloride (496 mg, 3.91 mmol, 342 μL, 1.2 eq.). The mixture was stirred at 0° C. for 2 hours. The mixture was concentrated under reduce pressure to get 3-(dimethylamino)propanoyl chloride (7) (560 mg, crude, HCl) as yellow oil.

### Step 5

To a solution of [7,7-dimethyl-8-oxo-8-(4-pentylnonoxy)octyl] (2S)-4-hydroxy-1-(6-oxo-6-undecoxy-hexyl)pyrrolidine-2-carboxylate (6) (0.5 g, 653 μmol, 1 eq.) in DCM (10 mL) was added TEA (660 mg, 6.53 mmol, 908 μL, 10 eq.) and DMAP (39.8 mg, 326 μmol, 0.5 eq.) and 3-(dimethylamino)propanoyl chloride (7) (505 mg, 2.94 mmol, 4.5 eq., HCl) at 0° C. The mixture was stirred at 25° C. for 8 hours. The reaction mixture was quenched by addition H₂O 20 mL at 0° C. and then extracted with EtOAc 30 mL (10 mL×3). The combined organic layers were over Na₂SO₄, filtered and concentrated under reduced pressure to give a residue. The residue was purified by column chromatography (SiO₂, Petroleum ether/Ethyl acetate=1/0 to 1/1, added 0.1% NH₃·H₂O). Then the mixture was purified by prep-HPLC (column: Phenomenex Luna C18 100×30 mm×5 μm; mobile phase: [water (HCl)-ACN]; B %: 50%-80%, 10 min) and worked-up with sat. NaHCO₃ (300 mL), extracted with EtOAc 30 mL (10 mL×3). The combined organic layers were over Na₂SO₄, filtered and concentrated under reduced pressure. Then it was purified by p-TLC (SiO₂, Petroleum ether/Ethyl acetate=1/1, added 0.1% NH₃·H₂O) to give compound 2366 ([7,7-dimethyl-8-oxo-8-(4-pentylnonoxy)octyl] (2S)-4-[3-(dimethylamino)propanoyloxy]-1-(6-oxo-6-undecoxy-hexyl)pyrrolidine-2-carboxy late) (96 mg, 416 μmol, 17% yield) as colorless oil.

¹H NMR (400 MHz, CDCl₃), 5.20-5.28 (m, 1H), 4.01-4.12 (m, 6H), 3.10-3.54 (m, 2H), 2.51-2.74 (m, 7H), 2.15-2.33 (m, 10H), 1.59-1.71 (m, 8H), 1.48-1.55 (m, 4H), 1.24-1.31 (m, 43H), 1.16 (s, 6H), 0.89 (t, J=6.8 Hz, 9H). MS (M+H⁺): 865.7.

### Example 7. Preparation of Lipid Nanoparticle Compositions with or without a Cargo

### Exemplary Lipid Nanoparticle Compositions.

Exemplary lipid nanoparticle compositions and comparative lipid nanoparticle compositions were prepared to result in an ionizable lipid:structural lipid:sterol:PEG-lipid at a molar ratio shown in the below charts. For instance, exemplary lipid nanoparticle compositions in this example are shown in the below chart. The exemplary ionizable lipids used for each exemplary lipid nanoparticle composition were Compounds 2243, 2335, 2331, and 2333 (LNP 2243, LNP 2335, LNP 2331, and LNP 2333).

### Comparative Lipid Nanoparticle Compositions.

Each exemplary lipid nanoparticle composition was compared against a comparative lipid nanoparticle composition that was otherwise the same, except that the comparative lipid did not contain the structure feature of

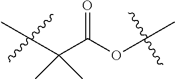 [SMILES: *OC(=O)C(*)(C)C]

in its lipid tails. The ionizable lipids used for each comparative lipid nanoparticle composition were Lipids 2141, 2233, 2231, and 2332 (LNP 2141, LNP 2233, LNP 2231 and LNP 2332)

Lipid
2243

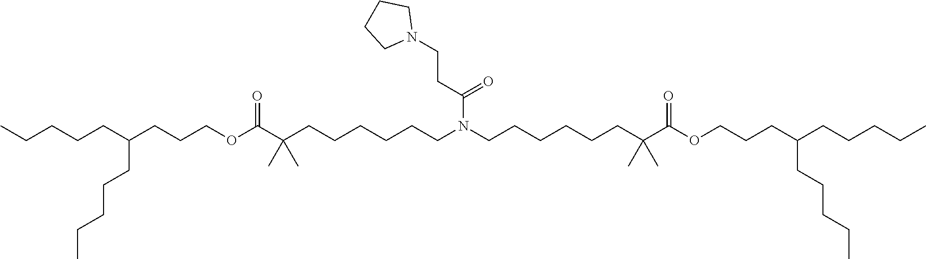 [SMILES: CCCCCC(CCCCC)CCCOC(=O)C(C)(C)CCCCCCN(CCCCCCC(C)(C)C(=O)OCCCC(CCCCC)CCCCC)C(=O)CCN1CCCC1]

2335

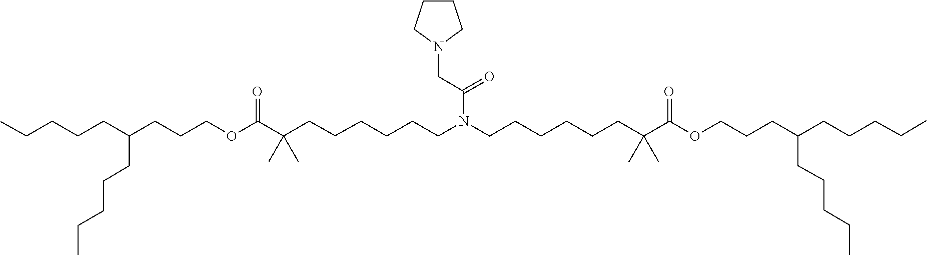 [SMILES: CCCCCC(CCCCC)CCCOC(=O)C(C)(C)CCCCCCN(CCCCCCC(C)(C)C(=O)OCCCC(CCCCC)CCCCC)C(=O)CN1CCCC1]

2331

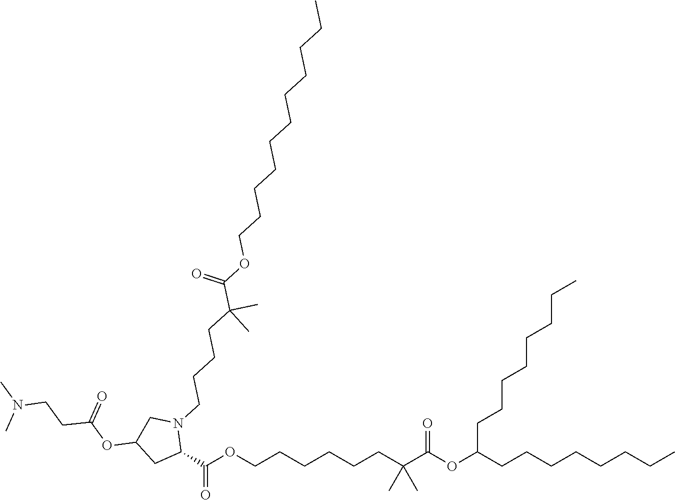 [SMILES: CCCCCCCCCCCOC(=O)C(C)(C)CCCCN1CC(OC(=O)CCN(C)C)C[C@H]1C(=O)OCCCCCCC(C)(C)C(=O)OC(CCCCCCCC)CCCCCCCC]

2333

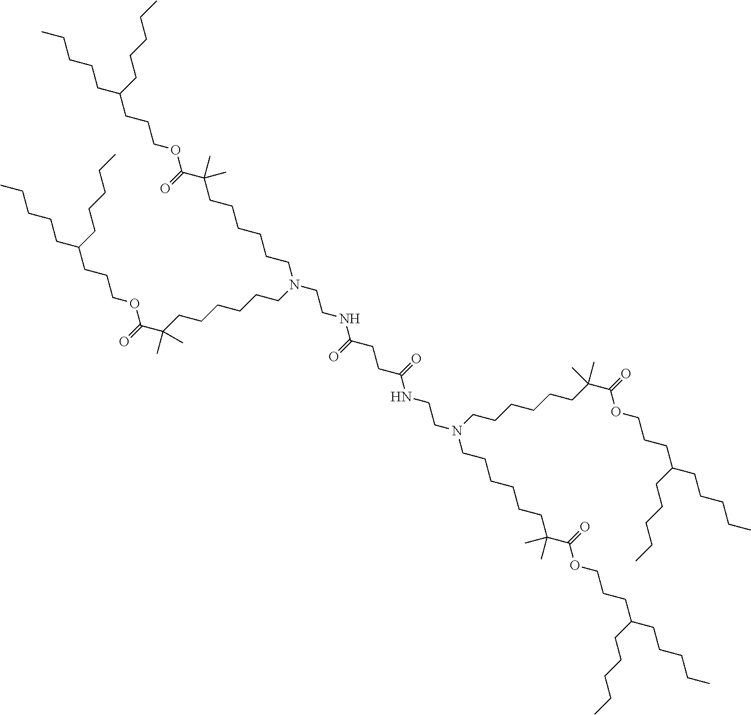 [SMILES: CCCCCC(CCCCC)CCCOC(=O)C(C)(C)CCCCCCN(CCCCCCC(C)(C)C(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC]

Comparative Lipid
2141

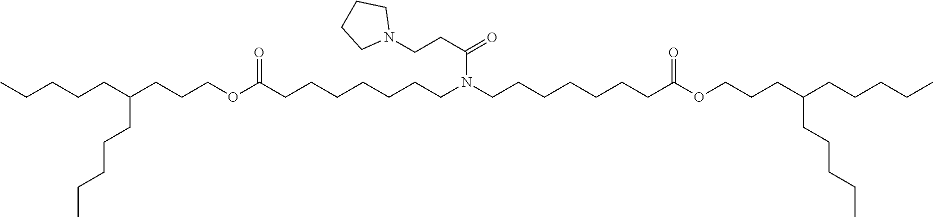 [SMILES: CCCCCC(CCCCC)CCCOC(=O)CCCCCCCN(CCCCCCCC(=O)OCCCC(CCCCC)CCCCC)C(=O)CCN1CCCC1]

2233

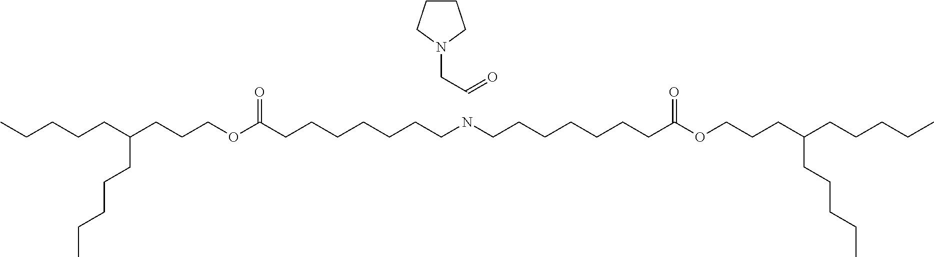

2231

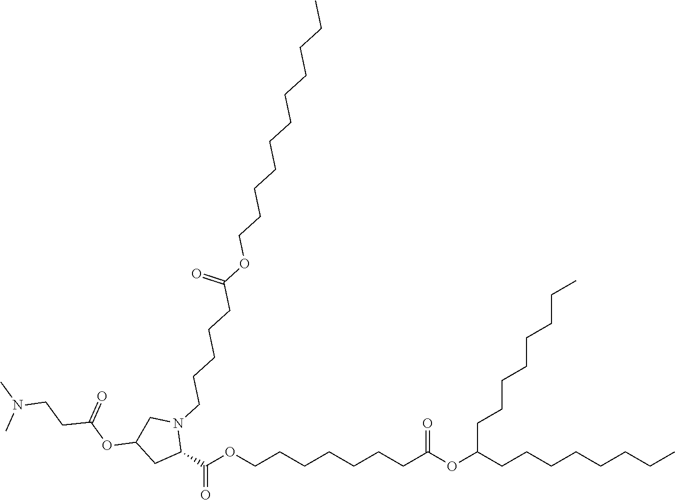 [SMILES: CCCCCCCCCCCOC(=O)CCCCCN1CC(OC(=O)CCN(C)C)C[C@H]1C(=O)OCCCCCCCC(=O)OC(CCCCCCCC)CCCCCCCC]

2332

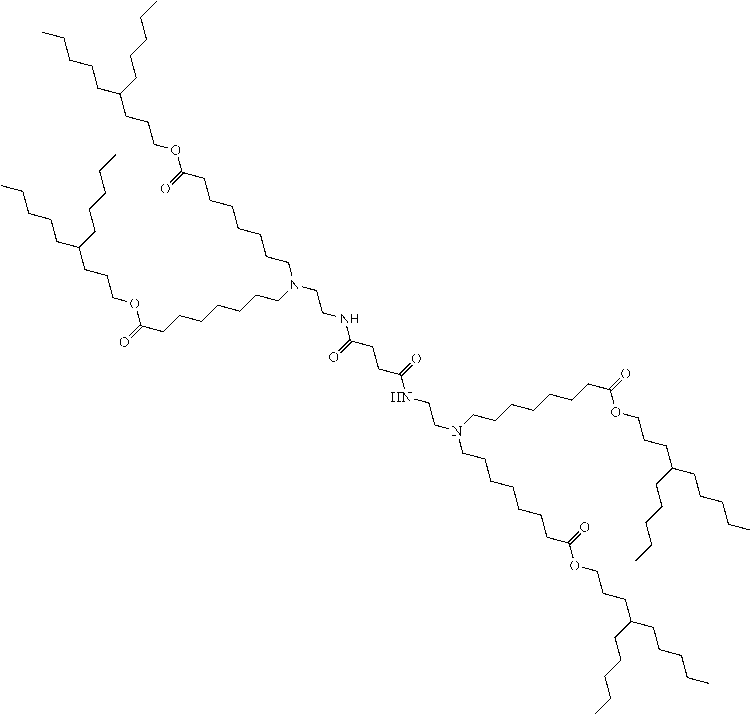 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCCC(=O)OCCCC(CCCCC)CCCCC)CCCCCCCC(=O)OCCCC(CCCCC)CCCCC]

| Lipids | |
|---|---|
| (LNP 2243, LNP 2335, LNP 2331) | |
| (LNP 2141, LNP 2233, LNP 2231) | Molar ratios |
| Exemplary (or comparative) ionizable | 50 |
| lipid | |
| DSPC | 10 |
| Cholesterol | 38.5 |
| DMPE−PEG | 1.5 |

| Lipids | |
|---|---|
| (LNP 2333) | |
| (LNP 2332) | Molar ratios |
| Exemplary (or comparative) ionizable | 35 |
| lipid | |
| DOPE | 16 |
| Cholesterol | 46.5 |
| DMPE−PEG | 2.5 |

To prepare these compositions, the lipids according to the above chart were solubilized in ethanol, mixed at the above molar ratios, and diluted in ethanol (organic phase) to obtain total lipid concentration of 5.5 mM.

Lipid Nanoparticle Compositions Encapsulating mRNA.

An mRNA solution (aqueous phase, fluc:EPO mRNA) was prepared with RNAse-free water and 100 mM citrate buffer pH 3 for a final concentration of 50 mM citrate buffer and 0.167 mg/mL mRNA concentration (1:1 Fluc:EPO). The formulations were maintained at an ionizable lipid to mRNA at an ionizable lipid nitrogen:mRNA phosphate (N:P) ratio of 6:1 for the exemplary and comparative lipid nanoparticle compositions (LNP 2243, LNP 2141, LNP 2335, LNP 2233, LNP 2331, LNP 2231, LNP 2333, and LNP 2332).

For each LNP composition, the lipid mix and mRNA solution were mixed at a 1:3 ratio by volume, respectively, on a NanoAssemblr Ignite (Precision Nanosystems) at a total flow rate of 9 mL/min. The resulting compositions were then loaded into Slide-A-Lyzer G2 dialysis cassettes (10k MWCO) and dialyzed in 200 times sample volume of 1×PBS for 2 hours at room temperature with gentle stirring. The PBS was refreshed, and the compositions were further dialyzed for at least 14 hours at 4° C., with gentle stirring. The dialyzed compositions were then collected and concentrated by centrifugation at 3000×g using Amicon Ultra centrifugation filters (100k MWCO). The concentrated particles were characterized for size, polydispersity, and particle concentration using Zetasizer Ultra (Malvern Panalytical) and for mRNA encapsulation efficiency using Quant-iT RiboGreen RNA Assay Kit (ThermoFisher Scientific).

For pKa measurement, a TNA assay was conducted according to those described in Sabnis et al., Molecular Therapy, 26(6):1509-19), which is incorporated herein by reference in its entirety. Briefly, 20 buffers (10 mM sodium phosphate, 10 mM sodium borate, 10 mM sodium citrate, and 150 mM sodium chloride, in distilled Water) of unique pH values ranging from 3.0-12.0 were prepared using IM sodium hydroxide and IM hydrochloric acid, 3.25 μL of a LNP composition (0.04 mg/mL mRNA, in PBS) was incubated with 2 μL of TNS reagent (0.3 mM, in DMSO) and 90 μL of buffer for each pH value (described above) in a 96-well black-walled plate. Each pH condition was performed in triplicate wells. The TNS fluorescence was measured using a Biotek Cytation Plate reader at excitation/emission wavelengths of 321/445 nm. The fluorescence values were then plotted and fit using a 4-parameter sigmoid curve. From the fit, the pH value yielding the half-maximal fluorescence was calculated and reported as the apparent LNP pKa value.

The particle characterization data for each exemplary and comparative lipid nanoparticle compositions (LNP 2243, LNP 2141, LNP 2335, LNP 2233, LNP 2331, LNP 2231, LNP 2333, and LNP 2332) are shown in the table below.

| | Size | | % entrapment | |
|---|---|---|---|---|
| LNP | (nm) | PDI | efficiency | pKa |
| 2243 | 87.8 | 0.04 | 94.9 | 7.1 |
| 2141 (comparative) | 73.8 | 0.06 | 95.9 | 7.57 |
| 2335 | 87.7 | 0.02 | 93.3 | 6.4 |
| 2233 (comparative) | 85.5 | 0.04 | 91.2 | 6.83 |
| 2331 | 90.0 | 0.04 | 97.4 | 5.41 |
| 2231 (comparative) | 91.6 | 0.05 | 93.9 | 5.03 |
| 2333 | 67.4 | 0.09 | 97.4 | — |
| 2332 (comparative) | 68.1 | 0.12 | 98 | 6.57 |

### Example 8. In Vivo Bioluminescent Imaging

The exemplary and comparative lipid nanoparticle compositions (LNP 2243, LNP 2141, LNP 2335, LNP 2233, LNP 2331, LNP 2231, LNP 2333, and LNP 2332) prepared according to Example 7, with encapsulating an mRNA (EPO), were used in this example.

### Bioluminescence Screening.

8-9 week old female Balb/c mice were utilized for bioluminescence-based ionizable lipid screening efforts. Mice were obtained from Jackson Laboratories (JAX Stock: 000651) and allowed to acclimate for one week prior to manipulations. Animals were placed under a heat lamp for a few minutes before introducing them to a restraining chamber. The tail was wiped with alcohol pads (Fisher Scientific) and, for each LNP composition described above, 100 μL of a lipid nanoparticle composition containing 10 μg total mRNA (5 μg Fluc+5 μg EPO) was injected intravenously using a 29G insulin syringe (Covidien), 4-6 hours post-dose, animals were injected with 200 μL of 15 mg/mL D-Luciferin (GoldBio), and placed in set nose cones inside the IVIS Lumina LT imager (PerkinElmer). LivingImage software was utilized for imaging. Whole body bio-luminescence was captured at auto-exposure after which animals are removed from the IVIS and placed into a CO₂ chamber for euthanasia. Cardiac puncture was performed on each animal after placing it in dorsal recumbency, and blood collection was performed using a 25G insulin syringe (BD). Once all blood samples were collected, tubes are spun at 2000G for 10 minutes using a tabletop centrifuge and plasma was aliquoted into individual Eppendorf tubes (Fisher Scientific) and stored at −80° C., for subsequent EPO quantification. EPO levels in plasma were determined using EPO MSD kit (Meso Scale Diagnostics).

hEPO MSD Measurement.

The reagents used for measuring hEPO levels included:
MSD wash buffer (#R61AA-1)
MSD EPO Kit (#K151VXK-2)
   MSD GOLD 96 Small Spot Streptavidin Plate
   Diluent 100
   Diluent 3
   Diluent 43
   Calibrator 9
   Capture Ab
   Detection Ab
   MSD GOLD Read Buffer B

General procedure. The Plate was coated. 200 μL of biotinylated capture antibody was added to 3.3 mL of Diluent 100 and was mixed by vortexing. 25 μL of the above solution was added to each well of the provided MSD GOLD Small Spot Streptavidin Plate. The plate was sealed with an adhesive plate seal and incubated with shaking at room temperature for 1 hour or at 2-8° C. overnight. The plate was washed 3 times with at least 150 μL/well of 1×MSD Wash Buffer.

Preparation of Calibrator Standards. The Calibrator vial(s) were brought to room temperature. Each vial of Calibrator was reconstituted by adding 250 μL of Diluent 43 to the glass vial, resulting in a 5× concentrated stock of the Calibrator. The reconstituted Calibrator was inverted at least 3 times, and equilibrated at room temperature for 15-30 minutes and then was vortexed briefly. Calibrator Standard 1 was prepared by adding 50 μL of the reconstituted Calibrator to 200 μL of Diluent 43 and vortexing. Calibrator Standard 2 was prepared by adding 75 μL of Calibrator Standard 1 to 225 μL of Diluent 43 and vortexing. The four-fold serial dilutions were repeated 5 additional times to generate a total of 7 Calibrator Standards. Mix by vortexing between each serial dilution. Diluent 43 was used as Calibrator Standard 8 (zero Calibrator).

Samples and Calibrators additions. 25 μL of Diluent 43 was added to each well. 25 μL of the prepared Calibrator Standard or sample was added to each well. The plate was sealed with an adhesive plate seal, and incubate at room temperature with shaking for 1 hour.

Preparation and addition of the Detection Antibody Solution. The detection antibody solution was provided as a 100× stock solution. The working solution was 1×. 60 μL of the supplied 100× detection antibody was added to 5940 μL of Diluent 3. The plate was washed 3 times with at least 150 μL/well of 1×MSD Wash Buffer. 50 μL of the Detection Antibody Solution prepared above was added to each well. The plate was sealed with an adhesive plate seal, and incubated at room temperature with shaking for 1 hour

Sample reading. The plate was washed 3 times with at least 150 μL/well of 1×MSD Wash Buffer. 150 μL of MSD GOLD Read Buffer B was added to each well. The plate was analyzed on an MSD instrument to read the EPO level.

The EPO levels determined by the in-vivo bioluminescent imaging for each exemplary and comparative lipid nanoparticle compositions (LNP 2243, LNP 2141, LNP 2335, LNP 2233, LNP 2331, LNP 2231, LNP 2333, and LNP 2332) are shown in the table below.

| | Bioluminescence | | | |
|---|---|---|---|---|
| | Whole | | | |
| LNP | body | Liver | Spleen | Spleen:Liver |
| 2243 | 1.60E+08 | 3.03E+07 | 4.18E+06 | 1.38E−01 |
| 2141 (comparative) | 1.93E+05 | 3.09E+04 | 1.29E+05 | 6.13E+00 |
| 2335 | 3.03E+08 | 4.32E+07 | 4.84E+06 | 1.15E−01 |
| 2233 (comparative) | 2.78E+08 | 3.93E+07 | 2.45E+06 | 4.39E−02 |
| 2331 | 6.75E+07 | 7.77E+06 | 3.46E+06 | 4.92E−01 |
| 2231 (comparative) | 2.42E+06 | 4.22E+05 | 3.77E+06 | 1.28E+01 |
| 2333 | 3.90E+08 | 5.62E+07 | 3.64E+05 | 6.31E−03 |
| 2332 (comparative) | 9.81E+07 | 7.77E+06 | 3.53E+05 | 4.52E−02 |

### Example 9: Synthesis of Exemplary Ionizable Lipid Compounds

### 9.1. Synthesis of Compound 2330

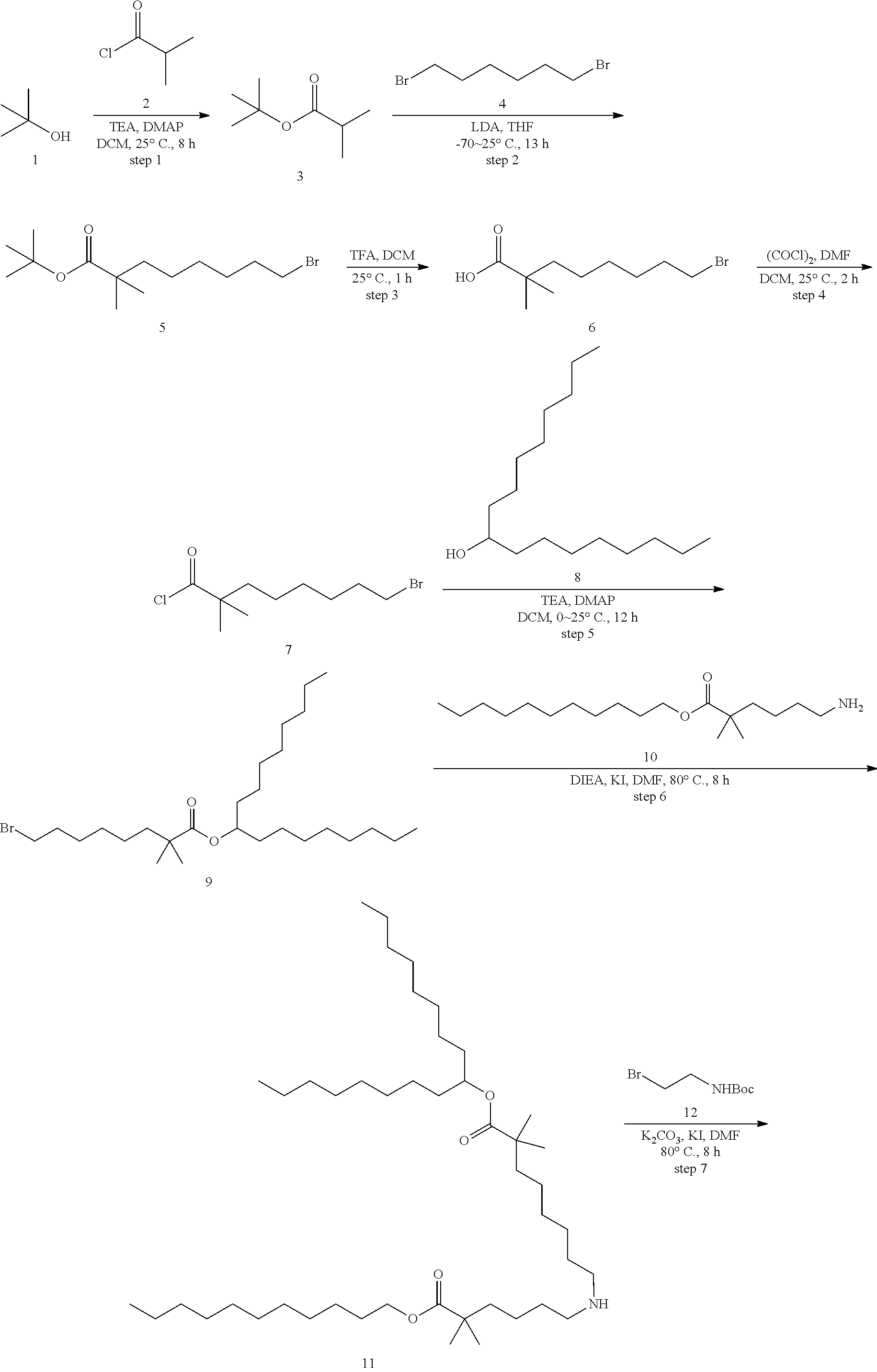

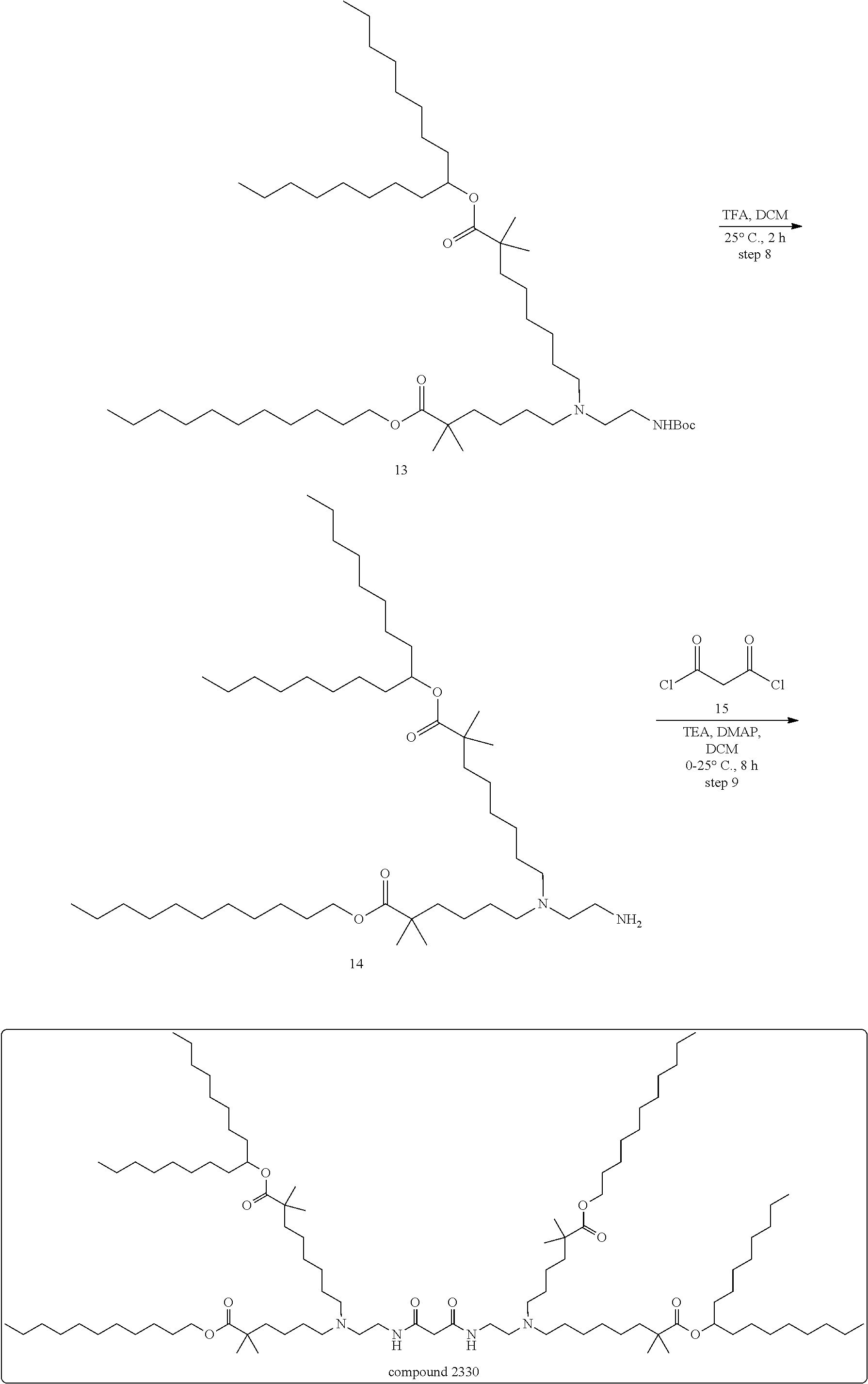 [SMILES: CCCCCCCCCCCOC(=O)C(C)(C)CCCCN(CCCCCCC(C)(C)C(=O)OC(CCCCCCCC)CCCCCCCC)CCNC(=O)OC(C)(C)C ; CCCCCCCCCCCOC(=O)C(C)(C)CCCCN(CCN)CCCCCCC(C)(C)C(=O)OC(CCCCCCCC)CCCCCCCC ; O=C(Cl)CC(=O)Cl ; CCCCCCCCCCCOC(=O)C(C)(C)CCCCN(CCCCCCC(C)(C)C(=O)OC(CCCCCCCC)CCCCCCCC)CCNC(=O)CC(=O)NCCN(CCCCCCC(C)(C)C(=O)OC(CCCCCCCC)CCCCCCCC)CCCCC(C)(C)C(=O)OCCCCCCCCCCC]

### Step 1:

To a solution of 2-methylpropanoyl chloride (204.0 g, 1.915 mol, 200 mL, 1 eq) in DCM (4000 mL) was added a solution of 2-methylpropan-2-ol (149.0 g, 2.010 mol, 192.3 mL, 1.05 eq) in DCM (4000 mL) and then TEA (290.6 g, 2.872 mmol, 399.7 mL, 1.5 eq) and DMAP (11.7 g, 95.7 mmol, 0.05 eq) was added into the mixture, the mixture was stirred at 25° C. for 8 h. The mixture was added into H₂O (5 L), extracted with DCM (3000 mL*2), organic layer was washed with brine (2000 mL*2), dried over Na₂SO₄, filtered and filtrate was concentrated under reduced pressure. The crude product was distilled in vacuum (100° C., 0.08 MPa/oil pump) to give compound Tert-butyl 2-methylpropanoate (200 g, 1.39 mol, 72.44% yield) as yellow oil.

### Step 2:

To a solution of N-isopropylpropan-2-amine (5.26 g, 52.01 mmol, 7.35 mL, 1.5 eq) in THF (250 mL) was added n-BuLi (2.5 M, 20.80 mL, 1.5 eq) at −40° C., under N₂, stirred for 0.5 h and then cooled to −70° C., the solution was added dropwise into a solution of tert-butyl 2-methylpropanoate (5 g, 34.67 mmol, 1 eq) in the THF (100 mL), stirred at −70° C. for 0.5 h under N₂, a solution of 1,6-dibromohexane (15.23 g, 62.41 mmol, 9.58 mL, 1.8 eq) in THF (100 mL) was added dropwise into the mixture at −70° C., the mixture was stirred at 25° C. for 12 h under N₂. The reaction mixture was cooled to 0° C., and then added slowly into aq. NH₄Cl solution (1000 mL) under N₂ at 0° C., the mixture was stirred at 0° C. for 0.5 h, then the mixture was extracted with EtOAc 900 mL (300 mL*3). The combined organic lavers were washed with sat, brine 450 mL (150 mL*3), dried over Na₂SO₄, filtered and concentrated under reduced pressure to give a residue. The residue was purified by column chromatography (SiO₂, Petroleum ether/Ethyl acetate=1/0 to 5/1) to give compound tert-butyl 8-bromo-2,2-dimethyl-octanoate (35 g, 113.91 mmol, 65.71% yield, 5 batches) as colorless oil.

### Step 3:

To a solution of tert-butyl 8-bromo-2,2-dimethyl-octanoate (14 g, 45.56 mmol, 1 eq) in DCM (80 mL) was added TFA (61.60 g, 540.24 mmol, 40 mL, 11.86 eq). The mixture was stirred at 25° C. for 1 hr. The reaction mixture was adjusted pH to 8 with sat. NaHCO₃, and then diluted with H₂O 500 mL and extracted with EtOAc 450 mL (150 mL*3). The combined organic layers were dried over Na₂SO₄, filtered and concentrated under reduced pressure to give a residue. The residue was purified by column chromatography (SiO₂, Petroleum ether/Ethyl acetate=1/0 to 1/1) to give compound 8-bromo-2,2-dimethyl-octanoic acid (17 g, 67.69 mmol, 85.00% yield, 4 batches) as yellow oil.

¹H NMR (400 MHz, CDCl₃), 3.40 (t, J=7.2 Hz, 2H), 1.83-1.87 (m, 2H), 1.52-1.54 (m, 2H), 1.41-1.49 (m, 2H), 1.28-1.31 (m, 4H), 1.25 (s, 6H).

### Step 4:

To a solution of 8-bromo-2,2-dimethyl-octanoic acid (8.5 g, 33.84 mmol, 1 eq) in DCM (100 mL) was added DMF (247.37 mg, 3.38 mmol, 260.39 μL, 0.1 eq) and (COCl), (8.59 g, 67.69 mmol, 5.92 mL, 2 eq). The mixture was stirred at 25° C. for 2 hr. The reaction mixture was concentrated under reduced pressure to give compound 8-bromo-2,2-dimethyl-octanoyl chloride (18 g, crude, 2 batches) was obtained as yellow oil.

### Step 5:

To a solution of heptadecan-9-ol (5 g, 19.50 mmol, 1 eq) in DCM (150 mL) was added TEA (9.86 g, 97.48 mmol, 13.57 mL, 5 eq) and DMAP (1.19 g, 9.75 mmol, 0.5 eq) and 8-bromo-2,2-dimethyl-octanoyl chloride (5.78 g, 21.45 mmol, 1.1 eq) in DCM (100 mL) at 0° C. The mixture was stirred at 25° C. for 12 hr. The reaction mixture was diluted with water 100 ml and extracted with EtOAc 90 mL (30 mL*3). The combined organic layers were dried over Na₂SO₄, filtered and concentrated under reduced pressure to give a residue. The residue was purified by column chromatography (SiO₂, Petroleum ether/Ethyl acetate=1/0 to 10/1) to give compound 1-octylnonyl 8-bromo-2,2-dimethyl-octanoate (11 g, 22.47 mmol, 38.41% yield, 3 batches) as colorless oil.

¹H NMR (400 MHz, CDCl₃), 4.80-4.86 (m, 1H), 3.37-3.53 (m, 2H), 1.84-1.86 (m, 2H), 1.42-1.53 (m, 8H), 1.26-1.30 (m, 28H), 1.52-1.58 (m, 6H), 1.05-1.11 (m, 6H), 0.88 (t, J=6.4 Hz, 6H).

### Step 6:

To a solution of undecyl 6-amino-2,2-dimethyl-hexanoate (2.9 g, 9.25 mmol, 1 eq) and 1-octylnonyl 8-bromo-2,2-dimethyl-octanoate (4.76 g, 9.71 mmol, 1.05 eq) in DMF (30 mL) was added KI (767.75 mg, 4.62 mmol, 0.5 eq) and DIEA (2.39 g, 18.50 mmol, 3.22 mL, 2 eq). The mixture was stirred at 80° C. for 8 hr. The reaction mixture diluted with by addition H₂O 50 mL, and then extracted with EtOAc 45 mL (15 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound 1-octylnonyl 8-[(5,5-dimethyl-6-oxo-6-undecoxy-hexyl)amino]-2,2-dimethyl-octanoate (2.3 g, 3.18 mmol, 34.43% yield) as yellow oil.

¹H NMR (400 MHz, CDCl₃), 4.80-4.86 (m, 1H), 4.04 (t, J=6.4 Hz, 2H), 2.55-2.60 (m, 4H), 1.61-1.62 (m, 2H), 1.47-1.54 (m, 14H), 1.25-1.45 (m, 46H), 1.14-1.16 (d, J=5.2 Hz, 12H), 0.88 (t, J=6.4 Hz, 9H).

### Step 7:

To a solution of 1-octylnonyl 8-[(5,5-dimethyl-6-oxo-6-undecoxy-hexyl)amino]-2,2-dimethyl-octanoate (1.6 g, 2.22 mmol, 1 eq), K₂CO₃ (1.53 g, 11.08 mmol, 5 eq) and KI (367.76 mg, 2.22 mmol, 1 eq) in DMF (50 mL) was added tert-butyl N-(2-bromoethyl) carbamate (2.48 g, 11.08 mmol, 5 eq). The mixture was degassed and purged with N₂ for 3 times, and then stirred at 80° C. for 8 hr under N₂ atmosphere. The reaction mixture was concentrated under reduced pressure to get a residue. Then crude product was added in H₂O 100 mL and extracted with EtOAc 150 mL (50 mL*3). The combined organic layers was washed with brine (150 mL*2), then dried over Na₂SO₄, filtered and the filtrate was concentrated under reduced pressure to give a residue. The residue was purified by column chromatography (SiO₂, Petroleum ether/Ethyl acetate=9/1 to 0/1) to give compound 1-octylnonyl 8-[2-(tert-butoxycarbonylamino)ethyl-(5,5-dimethyl-6-oxo-6-undecoxy-hexyl)amino]-2,2-dimethyl-octanoate (1 g, 1.16 mmol, 52.16% yield) as yellow oil.

¹H NMR (400 MHz, CDCl₃), 5.01 (s, 1H), 4.80-4.86 (m, 1H), 4.04 (t, J=6.8 Hz, 2H), 3.10-3.20 (m, 2H), 2.35-2.50 (m, 6H), 1.72 (s, 2H), 1.55-1.65 (m, 2H), 1.50-1.55 (m, 4H), 1.44 (s, 9H), 1.16-1.25 (m, 49H), 1.15 (s, 12H), 0.86-0.90 (m, 9H).

### Step 8:

A mixture of 1-octylnonyl 8-[2-(tert-butoxycarbonylamino)ethyl-(5,5-dimethyl-6-oxo-6-undecoxy-hexyl)amino]-2,2-dimethyl-octanoate (475 mg, 548.88 μmol, 1 eq) and TFA (4.62 g, 40.52 mmol, 3 mL, 73.82 eq) in DCM (6 mL) was degassed and purged with N₂ for 3 times, and then the mixture was stirred at 25° C. for 2 hr under N₂ atmosphere. The crude reaction mixture was concentrated under reduced pressure to get a residue. The crude product was diluted with EtOAc 20 mL, then the mixture was adjusted pH=7 with sat. NaHCO₃ aq, and washed with brine (20 mL*3). The combined organic layers were dried over Na₂SO₄, filtered and concentrated under reduced pressure to give a residue to give compound 1-octylnonyl 8-[2-aminoethyl-(5,5-dimethyl-6-oxo-6-undecoxy-hexyl)amino]-2,2-dimethyl-octanoate (420 mg, 548.82 μmol, 99.99% yield) as colorless oil was used for next step without purification.

### Step 9:

To a solution of 1-octylnonyl 8-[2-aminoethyl-(5,5-dimethyl-6-oxo-6-undecoxy-hexyl)amino]-2,2-dimethyl-octanoate (420 mg, 548.82 umol, 1 eq), TEA (166.60 mg, 1.65 mmol, 229.16 μL, 3 eq) and DMAP (33.52 mg, 274.41 μmol, 0.5 eq) in DCM (10 mL) was added a solution of propanedioyl dichloride (85.09 mg, 603.70 μmol, 58.68 μL, 1.1 eq) in DCM (10 mL) at 0° C. After addition, the mixture was stirred at 25° C. for 8 h under N₂ atmosphere. The mixture was added to sat. NaHCO₃ (20 mL), then extracted with DCM (20*3 mL). The combined organic layers were dried over Na₂SO₄, filtered and concentrated under reduced pressure to give a residue. The residue was purified by prep-TLC (SiO₂, PE:EA=3:2) and purified by column chromatography (SiO₂, Petroleum ether/Ethyl acetate=10/1 to 5/1, 3% NH₃·H₂O) to give compound 1-octylnonyl 8-[2-[[3-[2-[[7,7-dimethyl-8-(1-octylnonoxy)-8-oxo-octyl]-(5,5-dimethyl-6-oxo-6-undecoxy-hexyl)amino]ethylamino]-3-oxo-propanoyl]amino]ethyl-(5,5-dimethyl-6-oxo-6-undecoxy-hexyl)amino]-2,2-dimethyl-octanoate (70 mg, 43.79 μmol, 7.98% yield) as colorless oil.

¹H NMR (400 MHz, CDCl₃) 7.15 (brs, 1H), 4.80-4.86 (m, 2H), 4.05 (t, J=6.8 Hz, 4H), 3.15-3.39 (m, 7H), 2.40-2.56 (m, 11H), 1.62 (m, 6H), 1.45-1.55 (m, 16H), 1.35-1.45 (m, 6H), 1.20-1.40 (m, 96H), 1.16 (d, J=3.2 Hz, 24H), 0.86-0.91 (m, 18H).

LCMS (ELSD): (M+H⁺): 1598.4 @ 12.124 min.

### 9.2. Synthesis of 2367

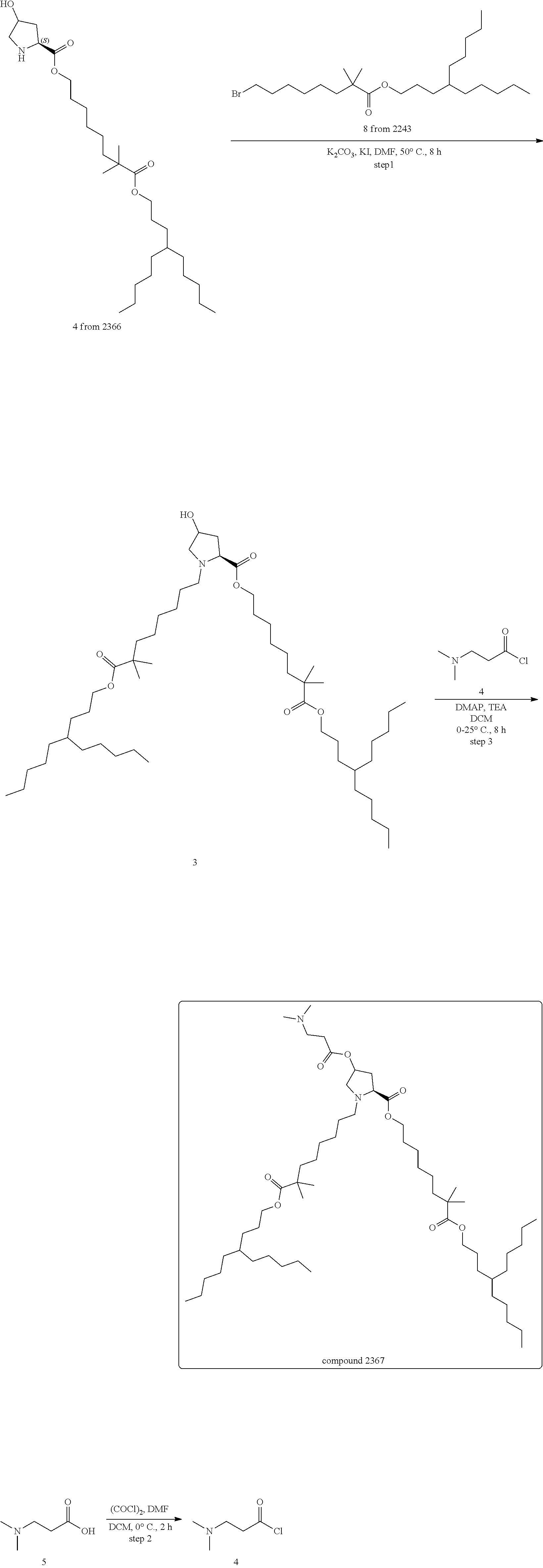

### Step 1:

To a solution of [7,7-dimethyl-8-oxo-8-(4-pentylnonoxy)octyl] (2S)-4-hydroxypyrrolidine-2-carboxylate (2 g, 4.02 mmol, 1 eq) in DMF (20 mL) was added K₂CO₃ (1.67 g, 12.05 mmol, 3 eq), KI (333.51 mg, 2.01 mmol, 0.5 eq) and 4-pentylnonyl 8-bromo-2,2-dimethyl-octanoate (2.70 g, 6.03 mmol, 1.5 eq). The mixture was stirred at 50° C. for 8 hr. The reaction mixture was quenched by addition H₂O 20 mL at 0° C., and then extracted with EtOAc 60 mL (20 mL*3). The combined organic layers were dried over Na₂SO₄, filtered and concentrated under reduced pressure to give a residue. The residue was purified by chromatography (SiO₂, Petroleum ether/Ethyl acetate=1/0 to 3/1, added NH₃·H₂O) to give compound [7,7-dimethyl-8-oxo-8-(4-pentylnonoxy)octyl] (2S)-1-[7,7-dimethyl-8-oxo-8-(4-pentylnonoxy)octyl]-4-hydroxy-pyrrolidine-2-carboxylate (2 g, 2.31 mmol, 57.59% yield) as yellow oil.

¹H NMR (400 MHz, CDCl₃), 4.27-4.50 (m, 1H), 4.01-4.14 (m, 6H), 3.40-3.66 (m, 2H), 3.08-3.26 (m, 1H), 2.26-2.75 (m, 3H), 2.02-2.05 (m, 1H), 1.49-1.64 (m, 17H), 1.24-1.31 (m, 46H), 1.16 (s, 12H), 0.89 (t, J=6.8 Hz, 12H).

### Step 2:

To a solution of 3-(dimethylamino) propanoic acid (0.5 g, 3.26 mmol, 1 eq, HCl) in DCM (20 mL) was added DMF (11.90 mg, 162.75 μmol, 12.52 μL, 0.05 eq) and oxalyl dichloride (495.78 mg, 3.91 mmol, 341.92 μL, 1.2 eq). The mixture was stirred at 0° C. for 2 hr. The mixture was concentrated under reduce pressure to give compound 3-(dimethylamino)propanoyl chloride (560 mg, crude, HCl) as yellow oil.

### Step 3:

To a solution of [7,7-dimethyl-8-oxo-8-(4-pentylnonoxy)octyl] (2S)-1-[7,7-dimethyl-8-oxo-8-(4-pentylnonoxy)octyl]-4-hydroxy-pyrrolidine-2-carboxylate (708.31 mg, 819.45 μmol, 1 eq) in DCM (10 mL) was added TEA (829.20 mg, 8.19 mmol, 1.14 mL, 10 eq) and DMAP (50.06 mg, 409.73 μmol, 0.5 eq) and 3-(dimethylamino)propanoyl chloride (0.5 g, 3.69 mmol, 4.5 eq) at 0° C. The mixture was stirred at 25° C. for 8 hr. The reaction mixture was quenched by addition H₂O 20 mL at 0° C., and then extracted with EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate=1/0 to 1/1, added NH₃·H₂O). Then purified by p-HPLC (column: Phenomenex Luna C18 100*30 mm*5 μm; mobile phase: [water (HCl)-ACN]; B %: 55%-85%, 10 min). Then the mixture was adjusted pH to 8 with sat. NaHCO₃, extracted with EtOAc 30 mL (10 mL*3). The combined organic layers were over Na₂SO₄, filtered and concentrated under reduced pressure to give compound [7,7-dimethyl-8-oxo-8-(4-pentylnonoxy)octyl] (2S)-4-[3-(dimethylamino)propanoyloxy]-1-[7,7-dimethyl-8-oxo-8-(4-pentylnonoxy)octyl]pyrrolidine-2-carboxylate (91 mg, 111.05 μmol, 13.55% yield) as yellow oil.

¹H NMR (400 MHz, CDCl₃), 5.19-5.28 (m, 1H), 4.01-4.12 (m, 6H), 3.08-3.55 (m, 2H), 2.30-2.67 (m, 7H), 2.02-2.30 (m, 8H), 1.57-1.62 (m, 6H), 1.47-1.52 (m, 6H), 1.24-1.31 (m, 50H), 1.15-1.16 (d, J=3.2 Hz, 12H), 0.89 (t, J=6.8 Hz, 12H).

LCMS: (M+H⁺): 963.8 @ 11.609&11.693 min.

### 9.3. Synthesis of 2368

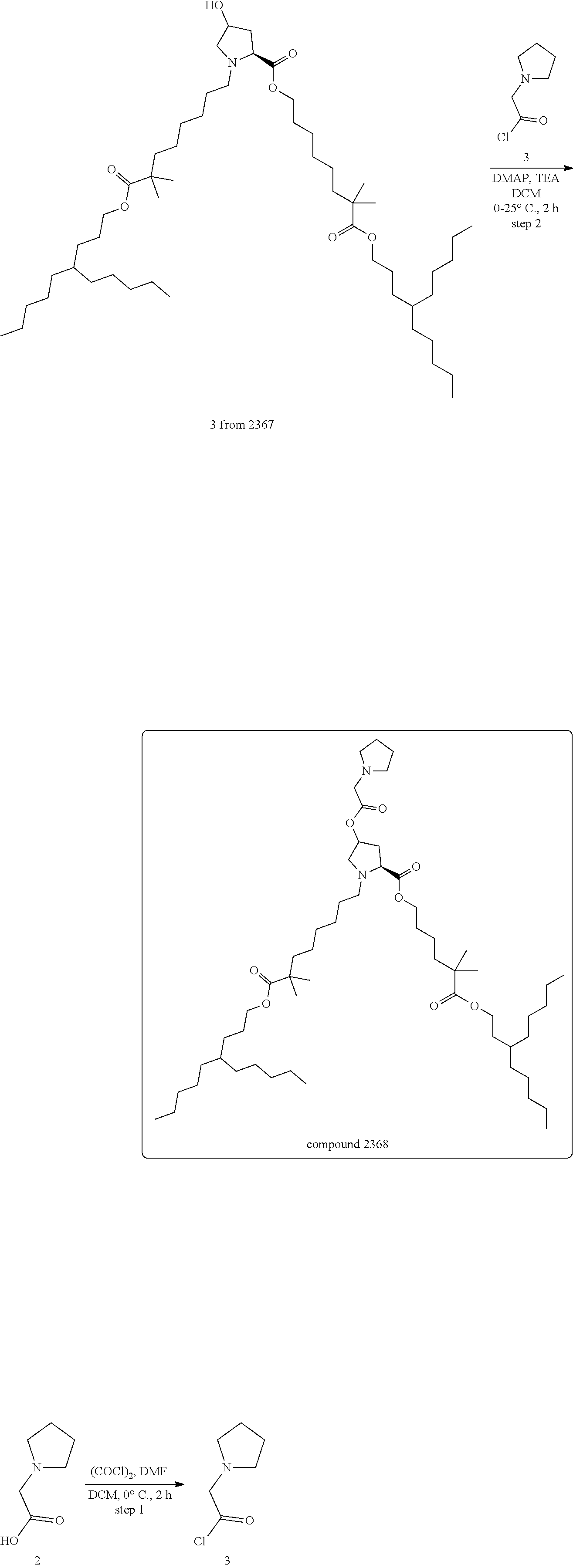

### Step 1:

To a solution of 2-pyrrolidin-1-ylacetic acid (350 mg, 2.71 mmol, 1 eq) in DCM (5 mL) was added DMF (9.90 mg, 135.49 μmol, 10.43 μL, 0.05 eq) and oxalyl dichloride (412.75 mg, 3.25 mmol, 284.65 μL, 1.2 eq). The mixture was stirred at 25° C. for 2 hr. The mixture was concentrated under reduce pressure to give compound 2-pyrrolidin-1-ylacetyl chloride (399 mg, crude) as yellow oil.

### Step 2:

To a solution of [7,7-dimethyl-8-oxo-8-(4-pentylnonoxy)octyl] (2S)-1-[7,7-dimethyl-8-oxo-8-(4-pentylnonoxy)octyl]-4-hydroxy-pyrrolidine-2-carboxylate (500 mg, 578.46 μmol, 1 eq) in DCM (10 mL) was added TEA (585.34 mg, 5.78 mmol, 805.14 μL, 10 eq) and DMAP (35.33 mg, 289.23 μmol, 0.5 eq) and 2-pyrrolidin-1-ylacetyl chloride (384.22 mg, 2.60 mmol, 4.5 eq) at 0° C. The mixture was stirred at 25° C. for 2 hr. The reaction mixture was quenched by addition H₂O 10 mL at 0° C., and then extracted with EtOAc 30 mL (10 mL*3). The combined organic layers were over Na₂SO₄, filtered and concentrated under reduced pressure to give a residue. The residue was purified by column chromatography (SiO₂, Petroleum ether/Ethyl acetate=1/0 to 1/1, added NH₃·H₂O). Then purified by p-HPLC (column: Phenomenex Luna C18 100*30 mm*5 μm; mobile phase: [water (HCl)-ACN]; B %: 60%-90%, 10 min). Then the mixture was adjusted pH to 8 with sat. NaHCO₃, extracted with EtOAc 30 mL (10 mL*3). The combined organic layers were dried over Na₂SO₄, filtered and concentrated under reduced pressure to give compound [7,7-dimethyl-8-oxo-8-(4-pentylnonoxy)octyl] (2S)-1-[7,7-dimethyl-8-oxo-8-(4-pentylnonoxy)octyl]-4-(2-pyrrolidin-1-ylacetyl)oxy-pyrrolidine-2-carboxylate (197 mg, 201.95 μmol, 34.91% yield) as yellow oil.

¹H NMR (400 MHz, CDCl₃), 5.23-5.32 (m, 1H), 4.01-4.11 (m, 6H), 3.09-3.55 (m, 4H), 2.04-2.78 (m, 9H), 1.86 (s, 4H), 1.59-1.62 (m, 6H), 1.47-1.52 (m, 6H), 1.24-1.31 (m, 50H), 1.40-1.43 (d, J=3.6 Hz, 12H), 0.88 (t, J=6.8 Hz, 12H). LCMS: (M+H⁺): 975.8 @ 11.746&11.940 min.

### 9.4. Synthesis of 2369

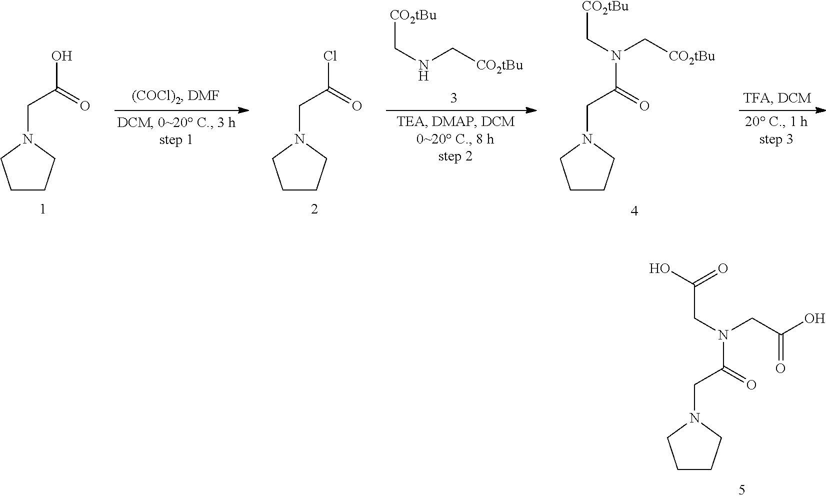 [SMILES: O=C(O)CN1CCCC1 ; O=C(Cl)CN1CCCC1 ; CC(C)(C)OC(=O)CNCC(=O)OC(C)(C)C ; CC(C)(C)OC(=O)CN(CC(=O)OC(C)(C)C)C(=O)CN1CCCC1 ; O=C(O)CN(CC(=O)O)C(=O)CN1CCCC1]

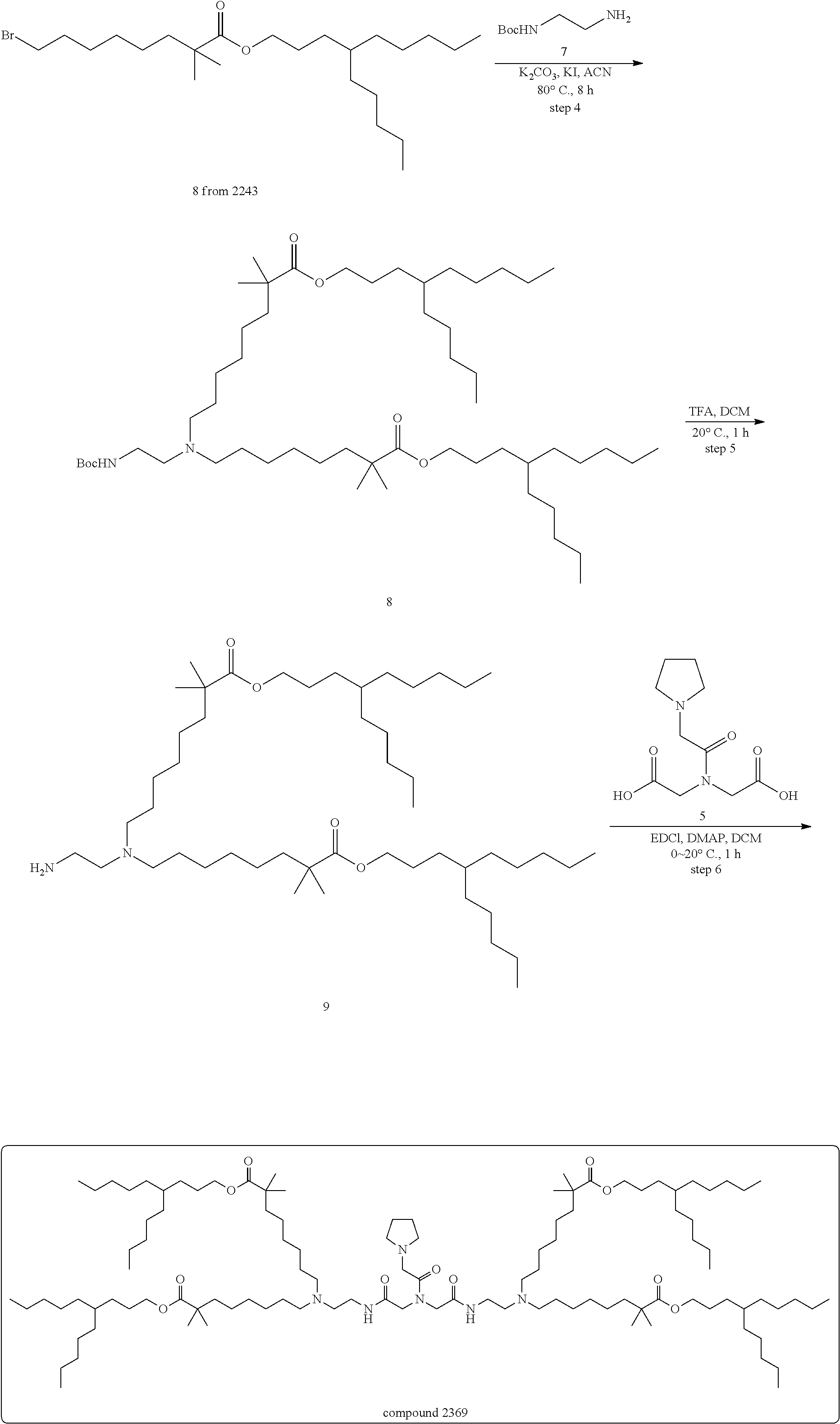

### Step 1:

A mixture of 2-pyrrolidin-1-ylacetic acid (1 g, 7.74 mmol, 1 eq) in DCM (10 mL) was added (COCl)₂ (4.91 g, 38.71 mmol, 3.39 mL, 5 eq), DMF (11.32 mg, 154.85 μmol, 11.91 μL, 0.02 eq) at 0° C. The mixture was stirred at 20° C. for 3 hr under N₂ atmosphere. The reaction mixture was concentrated under reduced pressure to give compound 2-pyrrolidin-1-ylacetyl chloride (1.1 g, crude) as yellow oil.

### Step 2:

To a solution of 2-pyrrolidin-1-ylacetyl chloride (2 g, 13.55 mmol, 1.2 eq), TEA (5.71 g, 56.46 mmol, 7.86 mL, 5 eq), DMAP (275.90 mg, 2.26 mmol, 0.2 eq) in DCM (10 mL) was added tert-butyl 2-[(2-tert-butoxy-2-oxo-ethyl)amino]acetate (2.77 g, 11.29 mmol, 1 eq) at 0° C. The mixture was stirred at 20° C. for 8 hr. The combined organic phase was diluted with EtOAc 20 mL and washed with water 60 mL (20 mL*3) and brine 40 mL (20 mL*2), dried with anhydrous Na₂SO₄, filtered and concentrated under reduced pressure to give a residue. The residue was purified by column chromatography (SiO₂, Petroleum ether/Ethyl acetate=1/0 to 5/1) to give compound tert-butyl 2-[(2-tert-butoxy-2-ovo-ethyl)-(2-pyrrolidin-1-ylacetyl)amino]acetate (2.3 g, 6.45 mmol, 57.14% yield) as yellow oil.

### Step 3:

To a solution of tert-butyl 2-[(2-tert-butoxy-2-oxo-ethyl)-(2-pyrrolidin-1-ylacetyl)amino]acetate (0.8 g, 2.24 mmol, 1 eq) in DCM (3 mL) was added TFA (1.54 g, 13.46 mmol, 1 mL, 6.00 eq). The mixture was stirred at 20° C. for 1 hr. The reaction mixture was concentrated under reduced pressure to get a residue. The residue was diluted with H₂O 10 mL and freeze-dried to give compound 2-[carboxymethyl-(2-pyrrolidin-1-ylacetyl)amino]acetic acid (0.5 g, 1.40 mmol, 62.18% yield, TFA) as yellow oil.

¹H NMR (400 MHz, CDCl₃), 9.93 (s, 1H), 4.34 (d, J=4.8 Hz, 2H), 4.18 (s, 2H), 4.07 (s, 2H), 3.56 (s, 2H), 2.98 (s, 2H), 1.86-1.98 (m, 4H).

### Step 4:

To a solution of 4-pentylnonyl 8-bromo-2,2-dimethyl-octanoate (6 g, 13.41 mmol, 2 eq), tert-butyl N-(2-aminoethyl) carbamate (1.07 g, 6.70 mmol, 1.06 mL, 1 eq) in ACN (10 mL) was added K₂CO₃ (1.85 g, 13.41 mmol, 2 eq), KI (556.39 mg, 3.35 mmol, 0.5 eq) and stirred at 80° C. for 8 hr. The reaction mixture was diluted with H₂O 20 mL and extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=10/1 to 1/1) to give compound 4-pentylnonyl 8-[2-(tert-butoxy carbonylamino)ethyl-[7,7-dimethyl-8-oxo-8-(4-pentylnonoxy)octyl]amino]-2,2-dimethyl-octanoate (5 g, 5.60 mmol, 83.48% yield) as a white solid.

¹H NMR (400 MHz, CDCl₃), 4.96 (s, 1H), 4.03 (t, J=6.4 Hz, 4H), 3.14 (d, J=3.2 Hz, 2H), 2.35-2.48 (m, 6H), 1.16-1.58 (m, 83H), 0.89 (t, J=6.8 Hz, 12H).

### Step 5:

To a solution of 4-pentylnonyl 8-[2-(tert-butoxycarbonylamino)ethyl-[7,7-dimethyl-8-oxo-8-(4-pentylnonoxy)octyl]amino]-2,2-dimethyl-octanoate (2 g, 2.24 mmol, 1 eq) in DCM (15 mL) was added TFA (7.68 g, 67.31 mmol, 5 mL, 30.07 eq). The mixture was stirred at 20° C. for 1 hr. The reaction mixture was concentrated under reduced pressure to get a residue. The residue was adjusted pH to 7 with saturated NaHCO₃ aqueous and extracted with EtOAc 30 mL (10 mL*3). The combined organic layers were dried over Na₂SO₄, filtered and concentrated under reduced pressure to give compound 4-pentylnonyl 8-[2-aminoethyl-[7,7-dimethyl-8-oxo-8-(4-pentylnonoxy)octyl]amino]-2,2-dimethyl-octanoate (1.5 g, 1.89 mmol, 84.46% yield) as yellow oil.

¹H NMR (400 MHz, CDCl₃), 4.03 (t, J=6.4 Hz, 4H), 2.84 (t, J=6.0 Hz, 2H), 2.60 (t, J=5.6 Hz, 2H), 2.49-2.57 (m, 4H), 1.48-1.50 (m, 12H), 1.16-1.31 (m, 62H), 0.89 (t, J=7.2 Hz, 12H).

### Step 6:

To a solution of 4-pentylnonyl 8-[2-aminoethyl-[7,7-dimethyl-8-oxo-8-(4-pentylnonoxy)octyl]amino]-2,2-dimethyl-octanoate (1.1 g, 1.39 mmol, 1 eq), EDCI (398.71 mg, 2.08 mmol, 1.5 eq), DMAP (84.70 mg, 693.27 μmol, 0.5 eq) in DCM (10 mL) was added 2-[carboxymethyl-(2-pyrrolidin-1-ylacetyl)amino]acetic acid (169.33 mg, 693.27 μmol, 0.5 eq) at 0° C. The mixture was stirred at 20° C. for 1 hr. The reaction mixture was diluted with H₂O 20 mL and extracted with EtOAc 60 mL (20 mL*3). The combined organic layers were dried over Na₂SO₄, filtered and concentrated under reduced pressure to give a residue. The residue was purified by column chromatography (SiO2, Petroleum ether/Ethyl acetate=1/0 to 2/1, added 3% NH₃·H₂O). Then the residue was purified by prep-HPLC (column: X-Select CSH Phenyl-Hexyl 100*30 5 u; mobile phase: [H₂O (0.04% HCl)-THF:ACN=1:3]; gradient: 55%-90% B over 10.0 min), then adjusted pH to 7 with saturated NaHCO₃ aqueous and extracted with EtOAc 30 mL (10 mL*3). The combined organic layers were dried over Na₂SO₄, filtered and concentrated under reduced pressure to give compound 4-pentylnonyl 8-[2-[[2-[[2-[2-[bis[7,7-dimethyl-8-oxo-8-(4-pentylnonoxy)octyl]amino]ethylamino]-2-oxo-ethyl]-(2-pyrrolidin-1-ylacetyl)amino]acetyl]amino]ethyl-[7,7-dimethyl-8-oxo-8-(4-pentylnonoxy)octyl]amino]-2,2-dimethyl-octanoate (262 mg, 140.06 μmol, 85.50% yield, 97.9% purity) as yellow oil.

¹H NMR (400 MHz, CDCl₃), 8.63 (t, J=5.2 Hz, 1H), 6.54 (t, J=4.4 Hz, 1H), 4.19 (s, 2H), 4.03 (t, J=6.4 Hz, 8H), 3.88 (s, 2H), 3.28-3.30 (m, 6H), 2.38-2.57 (m, 16H), 1.72-1.78 (m, 4H), 1.56-1.64 (m, 8H), 1.39-1.52 (m, 16H), 1.16-1.33 (m, 124H), 0.89 (t, J=6.8 Hz, 24H). LCMS: (½M+H⁺): 898.1 @ 10.525 min

### 9.5. Synthesis of 2370

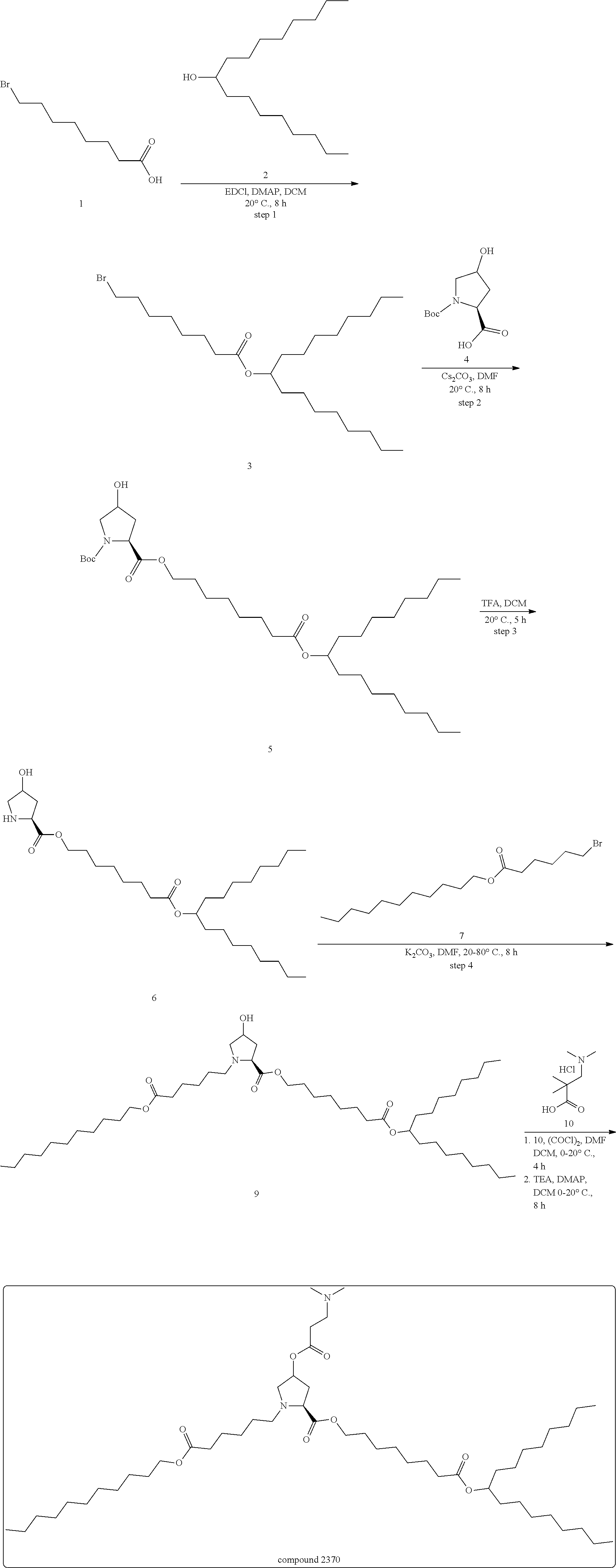

### Step 1:

To a solution of 8-bromooctanoic acid (5 g, 22.41 mmol, 1.2 eq) in DCM (50 mL) was added EDCl (5.37 g, 28.01 mmol, 1.5 eq), DMAP (456.31 mg, 3.74 mmol, 0.2 eq) and heptadecan-9-ol (4.79 g, 18.68 mmol, 1 eq) at 20° C. The mixture was degassed and purged with N₂ for 3 times, and then stirred at 20° C. for 8 hr under N₂ atmosphere. The reaction mixture was concentrated under reduced pressure to give a residue. The residue diluted with H₂O 500 mL then extracted with EtOAc 800 mL (400 mL*2). The combined organic layers were washed with brine 500 mL, dried over Na₂SO₄, filtered and concentrated under reduced pressure to give a residue. The residue was purified by column chromatography (SiO₂, Petroleum ether/Ethyl acetate=1/0 to 10/1) to give compound 1-octylnonyl 8-bromooctanoate (24 g, 52.00 mmol, 92.81% yield) as colourless oil.

### Step 2:

To a solution of 1-octylnonyl 8-bromooctanoate (5 g, 10.83 mmol, 1.2 eq) and (2S)-1-tert-butoxycarbonyl-4-hydroxy-pyrrolidine-2-carboxylic acid (2.09 g, 9.03 mmol, 1 eq) in DMF (70 mL) was added Cs₂CO₃ (6.47 g, 19.86 mmol, 2.2 eq) at 20° C. The mixture was degassed and purged with N₂ for 3 times, and then stirred at 20° C. for 8 hr under N₂ atmosphere. The reaction mixture was filtered and diluted with H₂O 50 mL then extracted with EtOAc 200 mL (100 mL*2). The combined organic layers were washed with brine 300 mL (150 mL*2), dried over Na₂SO₄, filtered and concentrated under reduced pressure to give a residue. The residue was purified by column chromatography (SiO₂, Petroleum ether/Ethyl acetate=8/1 to 3/1) to give compound O1-tert-butyl O2-[8-(1-octylnonoxy)-8-oxo-octyl] (2S)-4-hydroxypyrrolidine-1,2-dicarboxylate (27 g, 44.13 mmol, 97.76% yield) as colourless oil.

¹H NMR (400 MHz, CDCl₃), 4.85-4.89 (m, 1H), 4.11-4.55 (m, 4H), 3.35-3.75 (m, 2H), 2.05-2.35 (m, 4H), 1.55-1.63 (m, 10H), 1.26-1.50 (m, 37H), 0.88 (t, J=6.8 Hz, 6H)

### Step 3:

To a solution of O1-tert-butyl O2-[8-(1-octylnonoxy)-8-oxo-octyl] (2S)-4-hydroxypyrrolidine-1,2-dicarboxylate (10 g, 16.34 mmol, 1 eq) in DCM (60 mL) was added TFA (46.05 g, 403.87 mmol, 30 mL, 24.71 eq) at 20° C. The mixture was stirred at 20° C. for 5 hr. The reaction mixture was concentrated under reduced pressure to get a residue. The reaction mixture was adjusted to pH=7.0 with sat. NaHCO₃ aq, and extracted with EtOAc 100 mL (25 mL*4). The combined organic layers were dried over Na₂SO₄, filtered and concentrated under reduced pressure to give compound [8-(1-octylnonoxy)-8-oxo-octyl] (2S)-4-hydroxypyrrolidine-2-carboxylate (7.14 g, 13.95 mmol, 85.37% yield) as yellow oil.

### Step 4:

To a solution of [8-(1-octylnonoxy)-8-oxo-octyl] (2S)-4-hydroxypyrrolidine-2-carboxylate (7.14 g, 13.95 mmol, 1 eq) and undecyl 6-bromohexanoate (5.85 g, 16.74 mmol, 1.2 eq) in DMF (100 mL) was added K₂CO₃ (5.78 g, 41.85 mmol, 3 eq) at 20° C. The mixture was stirred at 80° C. for 8 h. The reaction mixture was filtered and the filtrate was concentrated under reduced pressure to give a residue. The reaction mixture was diluted with H₂O 300 mL and extracted with EtOAc 600 mL (200 mL*3). The combined organic layers were washed with brine 150 mL (50 ml*3), dried over Na₂SO₄, filtered and concentrated under reduced pressure to give a residue. The residue was purified by column chromatography (SiO₂, Petroleum ether/Ethyl acetate=5/1 to 0/1) to give compound [8-(1-octylnonoxy)-8-oxo-octyl] (2S)-4-hydroxy-1-(6-oxo-6-undecoxy-hexyl)pyrrolidine-2-carboxylate (9.6 g, crude) as yellow oil.

¹H NMR (400 MHz, CDCl₃), 4.76-4.82 (m, 1H), 4.22-4.52 (m, 1H), 4.10-4.20 (m, 2H), 4.07 (t, J=6.8 Hz, 2H), 3.40-3.68 (m, 1H), 3.02-3.24 (m, 1H), 2.45-2.78 (m, 3H), 2.25-2.33 (m, 4H), 1.86-2.17 (m, 2H), 1.51-1.56 (m, 8H), 1.42-1.44 (m, 6H), 1.19-1.38 (m, 48H), 0.80 (t, J=6.4 Hz, 9H).

### Step 5:

To a solution of 3-(dimethylamino)-2,2-dimethyl-propanoic acid (130.00 mg, 715.62 μmol, 1 eq, HCl) in DCM (8 mL) was added oxalyl dichloride (454.17 mg, 3.58 mmol, 313.22 μL, 5 eq) and DMF (19.00 mg, 259.94 μmol, 20.00 μL, 3.63e-1 eq) at 0° C. The mixture was degassed and purged with N₂ for 3 times, and then stirred at 20° C. for 4 hr under N₂ atmosphere. The reaction mixture was concentrated under reduced pressure to give compound 3-(dimethylamino)-2,2-dimethyl-propanoyl chloride (0.15 g, crude, HCl) as colourless oil. The crude oil residue wased dissolved with DCM (10 mL) and added into a solution of [8-(1-octylnonoxy)-8-oxo-octyl] (2S)-4-hydroxy-1-(6-oxo-6-undecoxy-hexyl)pyrrolidine-2-carboxylate (100.00 mg, 128.17 μmol, 1 eq), TEA (129.69 mg, 1.28 mmol, 178.40 μL, 10 eq) and DMAP (3.13 mg, 25.63 μmol, 0.2 eq) in DCM (7 mL) at 0° C. The mixture was degassed and purged with N₂ for 3 times, and then stirred at 20° C. for 8 hr under N₂ atmosphere. The reaction mixture was diluted with sat. NaHCO₃ aq. 20 mL and 50 extracted with EtOAc 100 mL (25 mL*4). The combined organic layers were dried over Na₂SO₄, filtered and concentrated under reduced pressure to give a residue. The residue was purified by column chromatography (SiO₂, Petroleum ether/Ethyl acetate=5/1 to 0/1, 5% NH₃·H₂O) and prep-TLC (SiO₂, Petroleum ether/Ethyl acetate=1:3, 2% NH₃·H₂O). The residue was extracted with hexane (5 mL) and ACN (5 mL). The hexane layer was concentrated under reduced pressure to give compound [8-(1-octylnonoxy)-8-oxo-octyl] (2S)-4-[3-(dimethylamino)-2,2-dimethyl-propanoyl]oxy-1-(6-oxo-6-undecoxy-hexyl)pyrrolidine-2-carboxylate (0.062 g, 68.33 μmol, 77.50% yield) as yellow oil.

¹H NMR (400 MHz, CDCl₃), 5.18-5.22 (m, 1H), 4.85-4.88 (m, 1H), 4.03-4.13 (m, 4H), 3.16-3.51 (m, 2H), 2.26-2.47 (m, 16H), 2.00-2.15 (m, 1H), 1.61-1.65 (m, 8H), 1.40-1.50 (m, 6H), 1.26-1.34 (m, 48H), 1.18 (s, 6H), 0.88 (t, J=6.4 Hz, 9H).

LCMS: (M+H⁺): 907.7 @ 11.518 min.

### 9.6. Synthesis of 2392

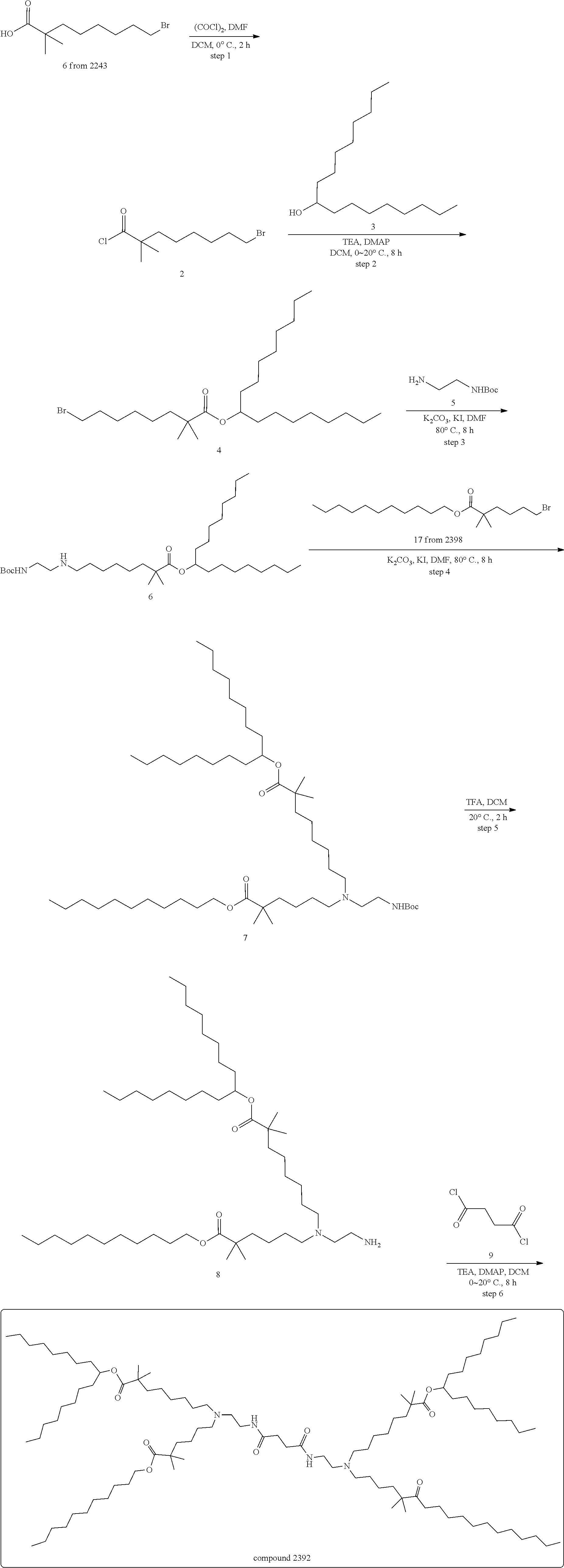

### Step 1:

To a solution of 8-bromo-2,2-dimethyl-octanoic acid (5 g, 19.91 mmol, 1 eq) in DCM (50 mL) was added DMF (72.76 mg, 995.38 μmol, 76.59 μL, 0.05 eq) and oxalyl dichloride (3.03 g, 23.89 mmol, 2.09 mL, 1.2 eq). The mixture was stirred at 0° C. for 2 hr. The mixture was concentrated under reduced pressure to give compound 8-bromo-2,2-dimethyl-octanoyl chloride (5.37 g, crude) as yellow oil.

### Step 2:

To a solution of heptadecan-9-ol (4.5 g, 17.55 mmol, 1 eq) in DCM (50 mL) was added TEA (5.33 g, 52.64 mmol, 7.33 mL, 3 eq) and DMAP (1.07 g, 8.77 mmol, 0.5 eq) and 8-bromo-2,2-dimethyl-octanoyl chloride (5.20 g, 19.30 mmol, 1.1 eq) at 0° C. The mixture was stirred at 20° C. for 8 hr. The reaction mixture was quenched by addition H₂O 50 mL at 0° C., and then extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=100/1 to 50/1) to give compound 1-octylnonyl 8-bromo-2,2-dimethyl-octanoate (4.8 g, 9.80 mmol, 55.87% yield) as yellow oil.

¹H NMR (400 MHz, CDCl₃), 4.80-4.87 (m, 1H), 3.39 (t, J=6.8 Hz, 2H), 1.82-1.86 (m, 2H), 1.43-1.53 (m, 8H), 1.26-1.30 (m, 28H), 1.15 (s, 6H), 0.88 (t, J=6.8 Hz, 6H).

### Step 3:

To a solution of 1-octylnonyl 8-bromo-2,2-dimethyl-octanoate (4.8 g, 9.80 mmol, 1 eq) in DMF (60 mL) was added KI (3.25 g, 19.61 mmol, 2 eq) and K₂CO₃ (4.06 g, 29.41 mmol, 3 eq) and tert-butyl N-(2-aminoethyl) carbamate (6.28 g, 39.21 mmol, 6.18 mL, 4 eq). The mixture was stirred at 80° C. for 8 hr. The reaction mixture was quenched by addition H₂O 50 mL at 0° C. and then extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=100/1 to 0/1) to give compound 1-octylnonyl 8-[2-(tert-butoxy carbonylamino)ethylamino]-2,2-dimethyl-octanoate (5 g, 8.79 mmol, 89.65% yield) as yellow oil.

¹H NMR (400 MHz, CDCl₃), 4.80-4.94 (m, 2H), 3.20-3.24 (m, 2H), 2.56-2.74 (m, 4H), 1.44-1.50 (m, 18H), 1.24-1.29 (m, 30H), 1.15 (s, 6H), 0.88 (t, J=6.4 Hz, 6H).

### Step 4:

To a solution of 1-octylnonyl 8-[2-(tert-butoxycarbonylamino)ethylamino]-2,2-dimethyl-octanoate (5 g, 8.79 mmol, 1 eq) in DMF (50 mL) was added K₂CO₃ (3.64 g, 26.37 mmol, 3 eq) and KI (2.92 g, 17.58 mmol, 2 eq) and undecyl 6-bromo-2,2-dimethyl-hexanoate (3.98 g, 10.55 mmol, 1.2 eq). The mixture was stirred at 80° C. for 8 hr. The reaction mixture was quenched by addition H₂O 60 mL at 0° C. and then extracted with EtOAc 150 mL (50 mL*3). The combined organic layers were dried over Na₂SO₄, filtered and concentrated under reduced pressure to give a residue. The residue was purified by chromatography (SiO₂, Petroleum ether/Ethyl acetate=50/1 to 0/1) to give compound 1-octylnonyl 8-[2-(tert-butoxy carbonylamino)ethyl-(5,5-dimethyl-6-oxo-6-undecoxy-hexyl)amino]-2,2-dimethyl-octanoate (6 g, 6.93 mmol, 78.89% yield) as colorless oil.

### Step 5:

To a solution of 1-octylnonyl 8-[2-(tert-butoxycarbonylamino)ethyl-(5,5-dimethyl-6-oxo-6-undecoxy-hexyl)amino]-2,2-dimethyl-octanoate (6 g, 6.93 mmol, 1 eq) in DCM (40 mL) was added TFA (20 mL). The mixture was stirred at 20° C. for 2 hr. The mixture was concentrated under reduced pressure, then adjust pH to 8 with sat. NaHCO₃, extracted with EtOAc 300 mL (100 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound 1-octylnonyl 8-[2-aminoethyl-(5,5-dimethyl-6-oxo-6-undecoxy-hexyl)amino]-2,2-dimethyl-octanoate (4 g, 5.23 mmol, 75.39% yield) as colorless oil.

¹H NMR (400 MHz, CDCl₃), 4.80-5.11 (m, 4H), 4.04 (t, J=6.4 Hz, 2H), 2.96 (t, J=5.6 Hz, 2H), 2.70 (t, J=6.0 Hz, 2H), 2.49-2.53 (m, 3H), 1.59-1.63 (m, 2H), 1.42-1.51 (m, 12H), 1.26-1.29 (m, 48H), 1.15-(d, J=2.4 Hz, 12H), 0.86-0.89 (m, 9H).

### Step 6:

To a solution of 1-octylnonyl 8-[2-aminoethyl-(5,5-dimethyl-6-oxo-6-undecoxy-hexyl)amino]-2,2-dimethyl-octanoate (1 g, 1.31 mmol, 2 eq) in DCM (10 mL) was added TEA (198.34 mg, 1.96 mmol, 272.81 μL, 3 eq), DMAP (39.91 mg, 326.68 μmol, 0.5 eq) and butanedioyl dichloride (101.26 mg, 653.35 μmol, 71.97 μL, 1 eq) at 0° C. The mixture was stirred at 20° C. for 8 hr. The reaction mixture was quenched by addition H₂O 10 mL at 0° C., and then extracted with EtOAc 30 mL (10 mL*3). The combined organic layers were dried over Na₂SO₄, filtered and concentrated under reduced pressure to give a residue. The residue was purified by p-HPLC (column: Welch Xtimate C1 250*50 mm*10 μm; mobile phase: [H₂O (0.05% HCl)-ACN:THF=1:1]; gradient: 35%-75% B over 20.0 min). The mixture was freeze-dried. Then the mixture was adjusted pH to 8 with sat. NaHCO₃, extracted with EtOAc 60 mL (20 mL*3). The combined organic layers were over Na₂SO₄, filtered and concentrated under reduced pressure. Then the residue was purified by prep-TLC (SiO₂, EtOAc:MeOH=10:1) to give compound 1-octylnonyl 8-[2-[[4-[2-[[7,7-dimethyl-8-(1-octylnonoxy)-8-oxo-octyl]-(5,5-dimethyl-6-oxo-6-undecoxy-hexyl)amino]ethylamino]-4-oxo-butanoyl]amino]ethyl-(5,5-dimethyl-6-oxo-6-undecoxy-hexyl)amino]-2,2-dimethyl-octanoate (23 mg, 14.26 μmol, 14.37% yield, 100% purity) as colorless oil.

¹H NMR (400 MHz, CDCl₃), 6.32 (t, J=4.8 Hz, 2H), 4.83 (t, J=6.8 Hz, 2H), 4.04 (t, J=6.8 Hz, 4H), 3.24-3.28 (m, 4H), 2.37-2.51 (m, 16H), 1.60-1.63 (m, 6H), 1.49-1.53 (m, 16H), 1.26-1.38 (m, 102H), 1.15 (d, J=3.2 Hz, 24H), 0.86-0.90 (m, 18H).

LCMS (CAD): (½M+H⁺): 807.0 @ 11.831 min. LCMS (ELSD): (½M+H⁺): 807.0 @ 11.047 min.

### 9.7. Synthesis of 2398

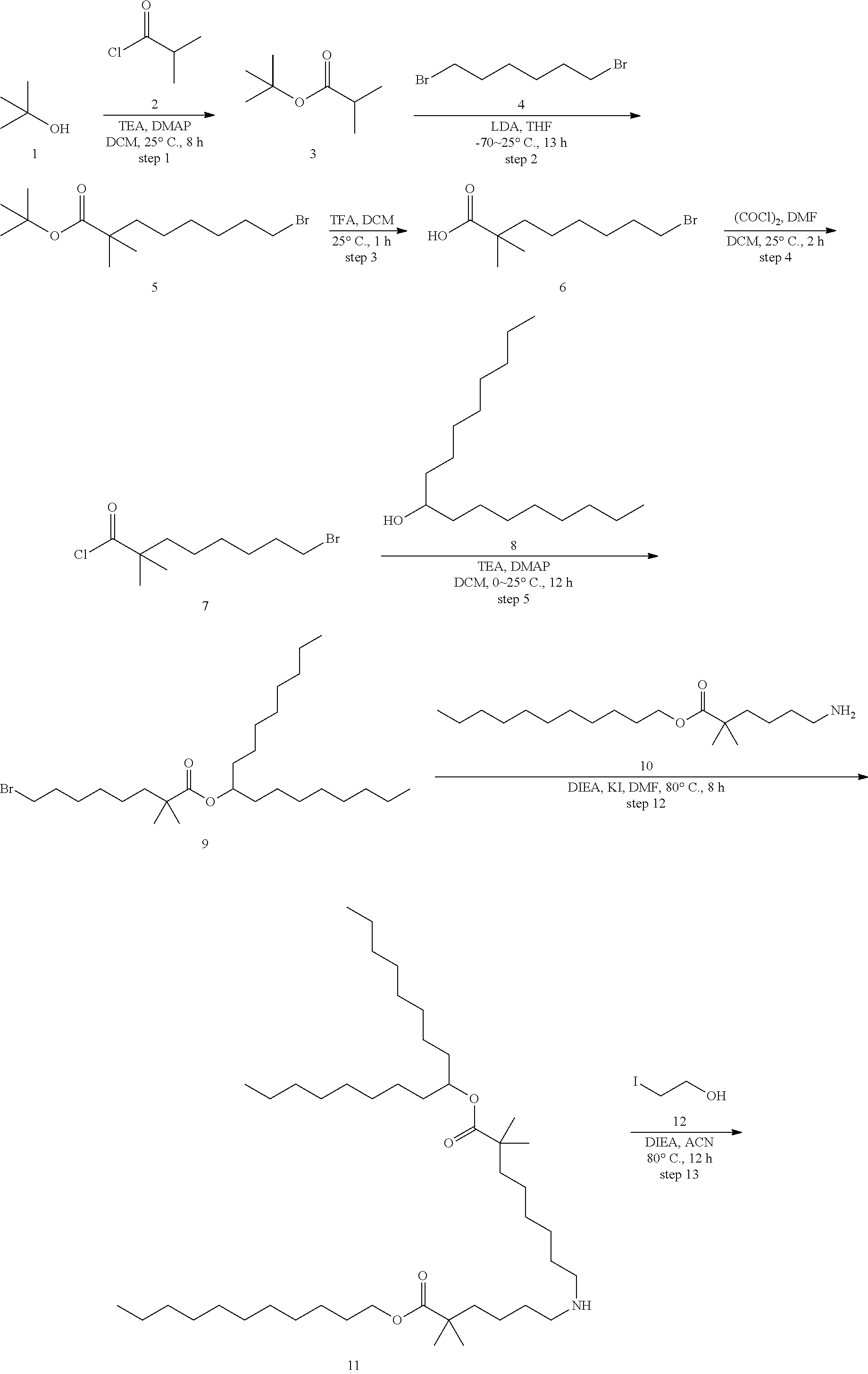

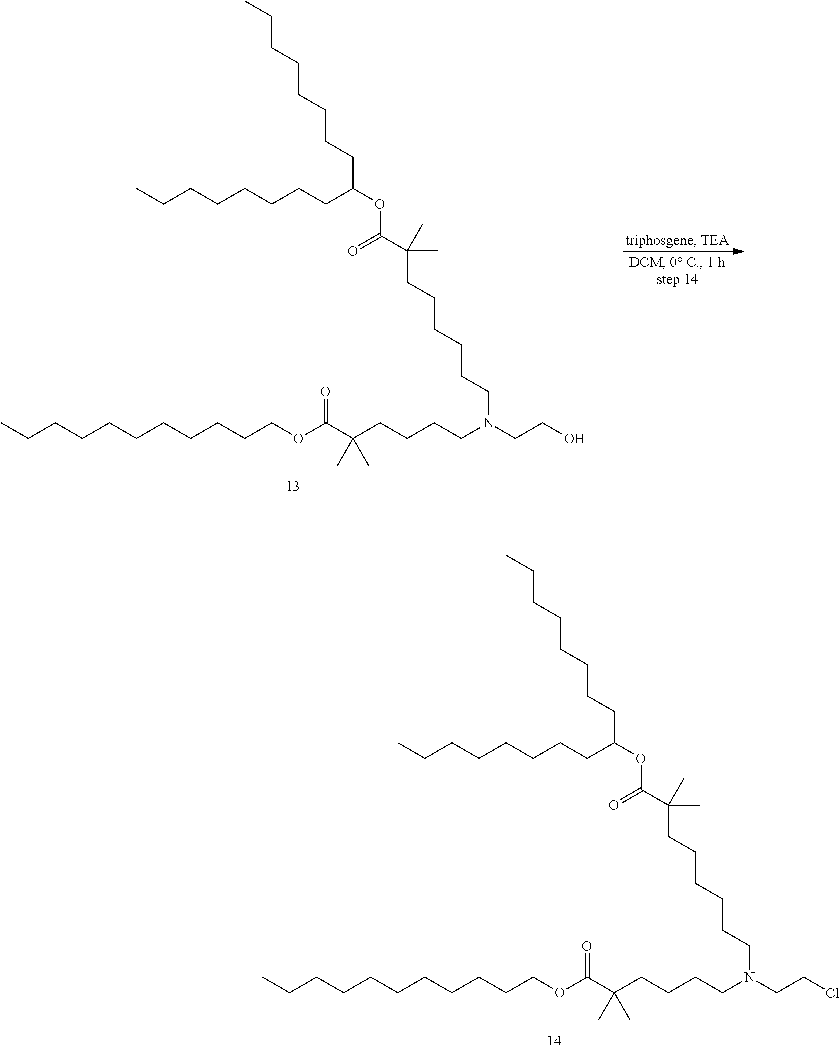 [SMILES: CCCCCCCCCCCOC(=O)C(C)(C)CCCCN(CCO)CCCCCCC(C)(C)C(=O)OC(CCCCCCCC)CCCCCCCC ; CCCCCCCCCCCOC(=O)C(C)(C)CCCCN(CCCl)CCCCCCC(C)(C)C(=O)OC(CCCCCCCC)CCCCCCCC]

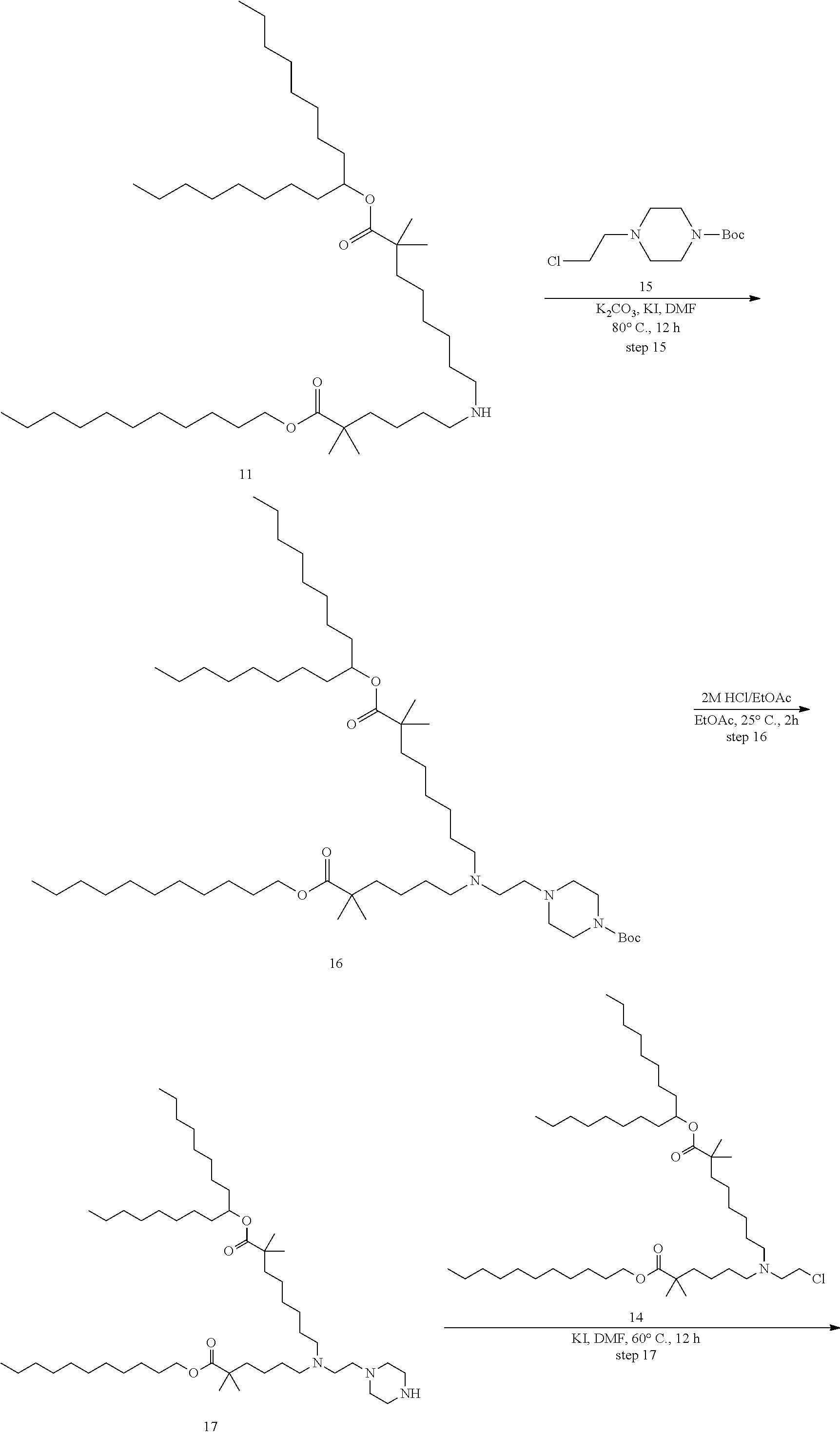 [SMILES: CCCCCCCCCCCOC(=O)C(C)(C)CCCCNCCCCCCC(C)(C)C(=O)OC(CCCCCCCC)CCCCCCCC ; CC(C)(C)OC(=O)N1CCN(CCCl)CC1 ; CCCCCCCCCCCOC(=O)C(C)(C)CCCCN(CCCCCCC(C)(C)C(=O)OC(CCCCCCCC)CCCCCCCC)CCN1CCN(C(=O)OC(C)(C)C)CC1 ; CCCCCCCCCCCOC(=O)C(C)(C)CCCCN(CCCCCCC(C)(C)C(=O)OC(CCCCCCCC)CCCCCCCC)CCN1CCNCC1 ; CCCCCCCCCCCOC(=O)C(C)(C)CCCCN(CCCl)CCCCCCC(C)(C)C(=O)OC(CCCCCCCC)CCCCCCCC]

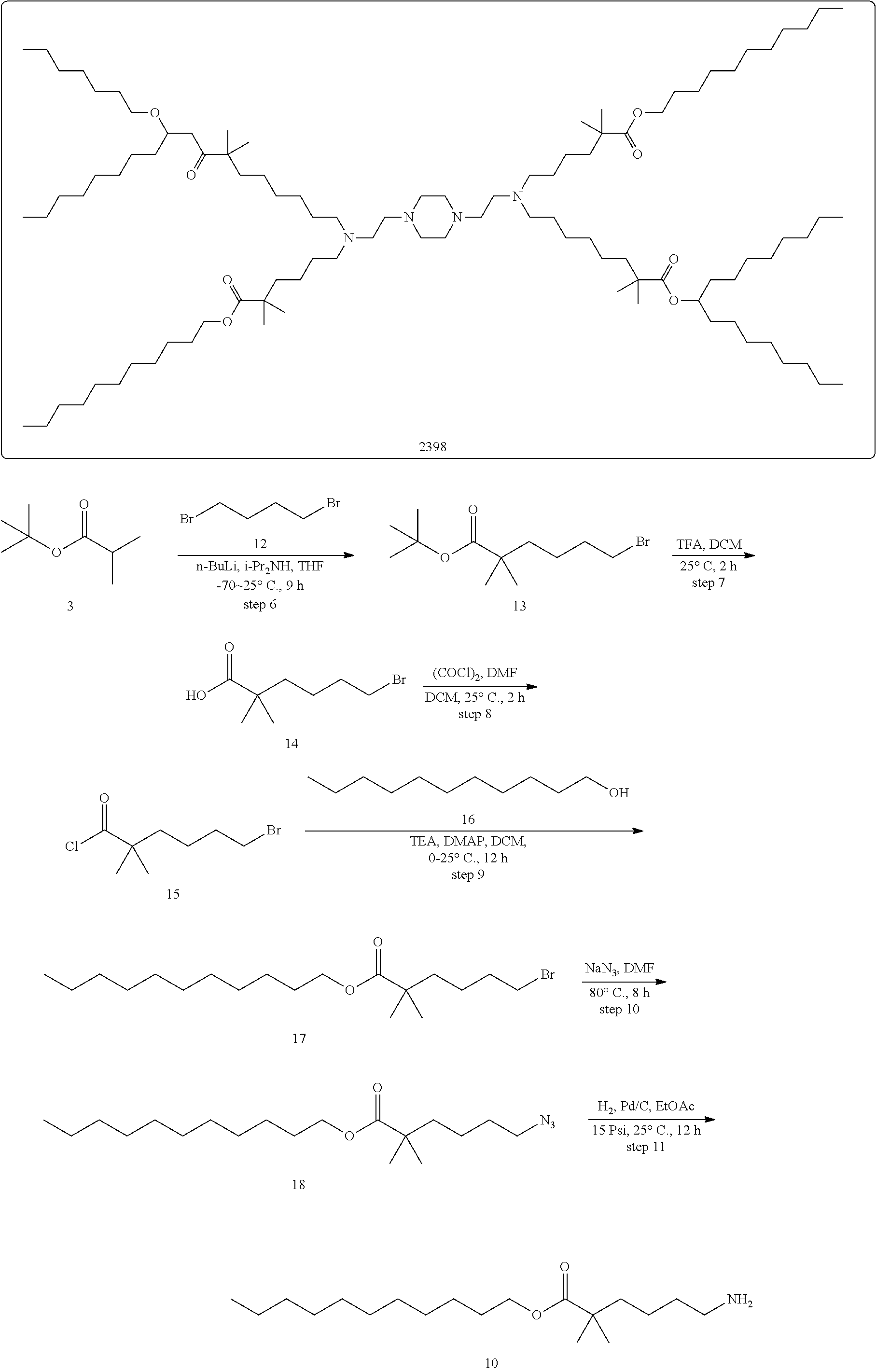

### Step 1:

To a solution of 2-methylpropanoyl chloride (204.0 g, 1.915 mol, 200 mL, 1 eq) in DCM (4000 mL) was added a solution of 2-methylpropan-2-ol (149.0 g, 2.010 mol, 192.3 mL, 1.05 eq) in DCM (4000 mL) and then TEA (290.6 g, 2.872 mmol, 399.7 mL, 1.5 eq) and DMAP (11.7 g, 95.7 mmol, 0.05 eq) was added into the mixture, the mixture was stirred at 25° C. for 8 h. The mixture was added into H₂O (5 L), extracted with DCM (3000 mL*2), organic layer was washed with brine (2000 mL*2), dried over Na₂SO₄, filtered and filtrate was concentrated under reduced pressure. The crude product was distilled in vacuum (100° C., 0.08 MPa/oil pump) to give compound Tert-butyl 2-methylpropanoate (200 g, 1.39 mol, 72.44% yield) as yellow oil.

### Step 2:

To a solution of N-isopropylpropan-2-amine (5.26 g, 52.01 mmol, 7.35 mL, 1.5 eq) in THF (250 mL) was added n-BuLi (2.5 M, 20.80 mL, 1.5 eq) at −40° C., under N₂, stirred for 0.5 h and then cooled to −70° C., the solution was added dropwise into a solution of tert-butyl 2-methylpropanoate (5 g, 34.67 mmol, 1 eq) in the THF (100 mL), stirred at −70° C. for 0.5 h under N₂, a solution of 1,6-dibromohexane (15.23 g, 62.41 mmol, 9.58 mL, 1.8 eq) in THF (100 mL) was added dropwise into the mixture at −70° C., the mixture was stirred at 25° C. for 12 h under N₂. The reaction mixture was cooled to 0° C., and then added slowly into aq. NH₄Cl solution (1000 mL) under N₂ at 0° C. the mixture was stirred at 0° C. for 0.5 h, then the mixture was extracted with EtOAc 900 mL (300 mL*3). The combined organic layers were washed with sat, brine 450 mL (150 mL*3), dried over Na₂SO₄, filtered and concentrated under reduced pressure to give a residue. The residue was purified by column chromatography (SiO₂, Petroleum ether/Ethyl acetate=1/0 to 5/1) to give compound tert-butyl 8-bromo-2,2-dimethyl-octanoate (35 g, 113.91 mmol, 65.71% yield, 5 batches) as colorless oil.

### Step 3:

To a solution of tert-butyl 8-bromo-2,2-dimethyl-octanoate (14 g, 45.56 mmol, 1 eq) in DCM (80 mL) was added TFA (61.60 g, 540.24 mmol, 40 mL, 11.86 eq). The mixture was stirred at 25° C. for 1 hr. The reaction mixture was adjusted pH to 8 with sat. NaHCO₃, and then diluted with H₂O 500 mL and extracted with EtOAc 450 mL (150 mL*3). The combined organic layers were dried over Na₂SO₄, filtered and concentrated under reduced pressure to give a residue. The residue was purified by column chromatography (SiO₂, Petroleum ether/Ethyl acetate=1/0 to 1/1) to give compound 8-bromo-2,2-dimethyl-octanoic acid (17 g, 67.69 mmol, 85.00% yield, 4 batches) as yellow oil.

¹H NMR (400 MHz, CDCl₃), 3.40 (t, J=7.2 Hz, 2H), 1.83-1.87 (m, 2H), 1.52-1.54 (m, 2H), 1.41-1.49 (m, 2H), 1.28-1.31 (m, 4H), 1.25 (s, 6H).

### Step 4:

To a solution of 8-bromo-2,2-dimethyl-octanoic acid (8.5 g, 33.84 mmol, 1 eq) in DCM (100 mL) was added DMF (247.37 mg, 3.38 mmol, 260.39 μL, 0.1 eq) and (COCl)₂ (8.59 g, 67.69 mmol, 5.92 mL, 2 eq). The mixture was stirred at 25° C. for 2 hr. The reaction mixture was concentrated under reduced pressure to give compound 8-bromo-2,2-dimethyl-octanoyl chloride (18 g, crude, 2 batches) was obtained as yellow oil.

### Step 5:

To a solution of heptadecan-9-ol (5 g, 19.50 mmol, 1 eq) in DCM (150 mL) was added TEA (9.86 g, 97.48 mmol, 13.57 mL, 5 eq) and DMAP (1.19 g, 9.75 mmol, 0.5 eq) and 8-bromo-2,2-dimethyl-octanoyl chloride (5.78 g, 21.45 mmol, 1.1 eq) in DCM (100 mL) at 0° C. The mixture was stirred at 25° C. for 12 hr. The reaction mixture was diluted with water 100 ml and extracted with EtOAc 90 mL (30 mL*3). The combined organic layers were dried over Na₂SO₄, filtered and concentrated under reduced pressure to give a residue. The residue was purified by column chromatography (SiO₂, Petroleum ether/Ethyl acetate=1/0 to 10/1) to give compound 1-octylnonyl 8-bromo-2,2-dimethyl-octanoate (11 g, 22.47 mmol, 38.41% yield, 3 batches) as colorless oil.

¹H NMR (400 MHz, CDCl₃), 4.80-4.86 (m, 1H), 3.37-3.53 (m, 2H), 1.84-1.86 (m, 2H), 1.42-1.53 (m, 8H), 1.26-1.30 (m, 28H), 1.52-1.58 (m, 6H), 1.05-1.11 (m, 6H), 0.88 (t, J=6.4 Hz, 6H).

### Step 6:

To a solution of n-BuLi (2.5 M, 104.02 mL, 1.5 eq) in THF (250 mL) was added dropwise diisopropylamine (26.31 g, 260.04 mmol, 36.75 mL, 1.5 eq) at −40° C., under N₂, stirred for 0.5 h and then cooled to −70° C., the solution was added dropwise into a solution of tert-butyl 2-methylpropanoate (25 g, 173.36 mmol, 1 eq) in THF (200 mL), stirred at −70° C. for 0.5 h under N₂, a solution of 1,4-dibromobutane (67.38 g, 312.05 mmol, 37.64 mL, 1.8 eq) in THF (200 mL) was added dropwise into the mixture at −70° C., the mixture was stirred at 25° C. for 8 h under N₂. The mixture was cooled to 0° C., and then added slowly into aq. NH₄Cl solution (200 mL) under N₂ at 0° C. The mixture was stirred at 0° C. for 30 min, then the mixture was extracted with EtOAc (200 mL*3). The combined organic phases were washed with brine (100 mL*2), dried over Na₂SO₄ and filtered and the filtrate was concentrated under reduced pressure. The residue was purified by column chromatography (SiO₂, Petroleum ether/Ethyl acetate=1/0 to 50/1) to give compound Tert-butyl 6-bromo-2,2-dimethyl-hexanoate (45 g, 161.17 mmol, 92.97% yield) as colorless oil.

### Step 7:

A solution of tert-butyl 6-bromo-2,2-dimethyl-hexanoate (10 g, 35.81 mmol, 1 eq) in DCM (30 mL) and TFA (50.84 g, 445.89 mmol, 33.01 mL, 12.45 eq) was stirred at 25° C. for 2 h. The mixture was concentrated under reduced pressure. And then the dissolved with EtOAc (200 mL), washed with NaHCO₃ (200 mL*3), dried over Na₂SO₄, filtered and the filtrate was concentrated to give compound 6-bromo-2,2-dimethyl-hexanoic acid (30 g, crude) as colorless oil.

### Step 8:

To a solution of 6-bromo-2,2-dimethyl-hexanoic acid (4 g, 17.93 mmol, 1 eq) in DCM (150 mL) was added DMF (131.04 mg, 1.79 mmol, 137.94 μL, 0.1 eq) and (COCl)₂ (4.55 g, 35.86 mmol, 3.14 mL, 2 eq). The mixture was stirred at 25° C. for 2 hr. The reaction mixture was concentrated under reduced pressure to give compound 6-bromo-2,2-dimethyl-hexanoyl chloride (17 g, crude, 4 batches) as a yellow solid.

### Step 9:

To a solution of undecan-1-ol (5 g, 29.02 mmol, 1 eq) in DCM (80 mL) was added TEA (14.68 g, 145.09 mmol, 20.19 mL, 5 eq) and DMAP (1.77 g, 14.51 mmol, 0.5 eq) and 6-bromo-2,2-dimethyl-hexanoyl chloride (7.71 g, 31.92 mmol, 1.1 eq) in DCM (50 mL) at 0° C. The mixture was stirred at 25° C. for 12 hr. The reaction mixture was diluted with water 100 mL and extracted with EtOAc 90 mL (30 mL*3). The combined organic layers were dried over Na₂SO₄, filtered and concentrated under reduced pressure to give a residue. The residue was purified by column chromatography (SiO₂, Petroleum ether/Ethyl acetate=1/0 to 5/1) to give compound undecyl 6-bromo-2,2-dimethyl-hexanoate (13 g, 34.45 mmol, 59.35% yield) as colorless oil.

¹H NMR (400 MHz, CDCl₃), 3.98 (t, J=6.4 Hz, 2H), 3.32 (t, J=6.8 Hz, 2H), 1.74-1.80 (m, 2H), 1.44-1.54 (m, 2H), 1.23-1.30 (m, 20H), 1.19 (s, 6H), 0.81 (t, J=6.4 Hz, 3H).

### Step 10:

To a solution of undecyl 6-bromo-2,2-dimethyl-hexanoate (13 g, 34.45 mmol, 1 eq) in DMF (150 mL) was added NaN₃ (11.26 g, 173.20 mmol, 5.03 eq). The mixture was stirred at 80° C. for 8 hr. The reaction mixture was diluted with water 200 mL and extracted with EtOAc 210) mL (70 mL*3). The combined organic layers were washed with sat, brine 90 mL (30 mL*3), dried over Na₂SO₄, filtered and concentrated under reduced pressure to give compound undecyl 6-azido-2,2-dimethyl-hexanoate (11.7 g, crude) as colorless oil.

### Step 11:

To a solution of Pd/C (6 g, 10% purity) in EtOAc (200 mL) was added undecyl 6-azido-2,2-dimethyl-hexanoate (11.7 g, 34.46 mmol, 1 eq). The mixture was stirred at 25° C. for 12 hr under H₂ atmosphere (15 psi). The reaction mixture was filtered and the filtrate was concentrated under pressure to give a residue. The residue was purified by column chromatography (SiO₂, Petroleum ether/Ethyl acetate=1/1 to dichloromethane/methyl alcohol=3/1) to give compound undecyl 6-amino-2,2-dimethyl-hexanoate (2.9 g, 9.25 mmol, 30.53% yield) as yellow oil.

¹H NMR (400 MHz, CDCl₃), 4.04 (t, J=6.4 Hz, 2H), 2.69 (t, J=6.8 Hz, 2H), 1.61-1.70 (m, 2H), 1.50-1.54 (m, 2H), 1.39-1.48 (m, 2H), 1.24-1.31 (m, 18H), 1.16 (s, 6H), 0.88 (t, J=6.4 Hz, 3H).

### Step 12:

To a solution of undecyl 6-amino-2,2-dimethyl-hexanoate (2.9 g, 9.25 mmol, 1 eq) and 1-octylnonyl 8-bromo-2,2-dimethyl-octanoate (4.76 g, 9.71 mmol, 1.05 eq) in DMF (30 mL) was added KI (767.75 mg, 4.62 mmol, 0.5 eq) and DIEA (2.39 g, 18.50 mmol, 3.22 mL, 2 eq). The mixture was stirred at 80° C. for 8 hr. The reaction mixture diluted with by addition H₂O 50 mL, and then extracted with EtOAc 45 mL (15 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound 1-octylnonyl 8-[(5,5-dimethyl-6-oxo-6-undecoxy-hexyl)amino]-2,2-dimethyl-octanoate (2.3 g, 3.18 mmol, 34.43% yield) as yellow oil.

¹H NMR (400 MHz, CDCl₃), 4.80-4.86 (m, 1H), 4.04 (t, J=6.4 Hz, 2H), 2.55-2.60 (m, 4H), 1.61-1.62 (m, 2H), 1.47-1.54 (m, 14H), 1.25-1.45 (m, 46H), 1.14-1.16 (d, J=5.2 Hz, 12H), 0.88 (t, J=6.4 Hz, 9H).

### Step 13:

To a solution of 1-octylnonyl 8-[(5,5-dimethyl-6-oxo-6-undecoxy-hexyl)amino]-2,2-dimethyl-octanoate (1.2 g, 1.66 mmol, 1 eq) in ACN (10 mL) was added DIEA (429.49 mg, 3.32 mmol, 578.83 μL, 2 eq) and 2-iodoethanol (428.59 mg, 2.49 mmol, 194.81 μL, 1.5 eq) in sequence. Then the mixture was stirred at 80° C. for 12 hr. The reaction mixture was concentrated under reduced pressure to remove solvent. The residue was purified by column chromatography (SiO₂, Petroleum ether/Ethyl acetate=20/1 to 1/1) to give compound 1-octylnonyl 8-[(5,5-dimethyl-6-oxo-6-undecoxy-hexyl)-(2-hydroxyethyl)amino]-2,2-dimethyl-octanoate (600 mg, 783.02 μmol, 47.13% yield) as colorless oil.

### Step 14:

To a solution of 1-octylnonyl 8-[(5,5-dimethyl-6-oxo-6-undecoxy-hexyl)-(2-hydroxyethyl)amino]-2,2-dimethyl-octanoate (600 mg, 783.02 μmol, 1 eq) was added TEA (118.85 mg, 1.17 mmol, 163.48 μL, 1.5 eq) and then a solution of triphosgene (302 mg, 1.02 mmol, 1.30 eq) in DCM (10 mL) was added into the mixture. The mixture was stirred at 0° C. for 1 hr under N₂. The reaction mixture was quenched by addition H₂O 50 mL at 0° C. and then extracted with EtOAc 45 mL (15 mL*3). The combined organic layers were dried over Na₂SO₄, filtered and concentrated under reduced pressure to give a residue. The residue was purified by column chromatography (SiO₂, Petroleum ether/Ethyl acetate=1/0 to 20/1) to give compound 1-octylnonyl 8-[2-chloroethyl-(5,5-dimethyl-6-oxo-6-undecoxy-hexyl)amino]-2,2-dimethyl-octanoate (300 mg, 382.31 μmol, 48.82% yield) as yellow oil.

### Step 15:

To a solution of 1-octylnonyl 8-[(5,5-dimethyl-6-oxo-6-undecoxy-hexyl)amino]-2,2-dimethyl-octanoate (1 g, 1.38 mmol, 1 eq) and tert-butyl 4-(2-chloroethyl) piperazine-1-carboxylate (378.87 mg, 1.52 mmol, 1.1 eq) in DMF (10 mL) was added KI (114.93 mg, 692.31 μmol, 0.5 eq) and K₂CO₃ (287.04 mg, 2.08 mmol, 1.5 eq) in sequence. Then the mixture was stirred at 80° C. for 12 hr. The reaction mixture diluted with by addition H₂O 30 mL, and then extracted with EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate=10/1 to 1/1) to give compound tert-butyl 4-[2-[[7,7-dimethyl-8-(1-octylnonoxy)-8-oxo-octyl]-(5,5-dimethyl-6-oxo-6-undecoxy-hexyl)amino]ethyl]piperazine-1-carboxylate (1 g, 941.68 μmol, 68.01% yield, 88% purity) as yellow oil.

### Step 16:

To a solution of tert-butyl 4-[2-[[7,7-dimethyl-8-(1-octylnonoxy)-8-oxo-octyl]-(5,5-dimethyl-6-oxo-6-undecoxy-hexyl)amino]ethyl]piperazine-1-carboxylate (1 g, 1.07 mmol, 1 eq) in EtOAc (5 mL) was added HCl/EtOAc (4 M, 5 mL, 18.69 eq) in sequence. Then the mixture was stirred at 25° C. for 2 hr. The reaction mixture was adjusted pH to 8 with sat. NaHCO₃ and then extracted EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate=1/1 to DCM:MeOH=3:1) to give compound 1-octylnonyl 8-[(5,5-dimethyl-6-oxo-6-undecoxy-hexyl)-(2-piperazin-1-ylethyl)amino]-2,2-dimethyl-octanoate (600 mg, 719.09 μmol, 67.20% yield, 100% purity) as yellow oil.

¹H NMR (400 MHz, CDCl₃), 4.80-4.86 (m, 1H), 4.04 (t, J=6.4 Hz, 2H), 2.92-2.94 (m, 4H), 2.05-2.64 (m, 14H), 1.61-1.63 (m, 2H), 1.45-1.52 (m, 10H), 1.26-1.31 (m, 44H), 1.15 (d, J=2.8 Hz, 12H), 0.88 (t, J=7.2 Hz, 9H).

### Step 17:

To a solution of 1-octylnonyl 8-[(5,5-dimethyl-6-oxo-6-undecoxy-hexyl)-(2-piperazin-1-ylethyl)amino]-2,2-dimethyl-octanoate (193.33 mg, 231.70 μmol, 1 eq) and 1-octylnonyl 8-[2-chloroethyl-(5,5-dimethyl-6-oxo-6-undecoxy-hexyl)amino]-2,2-dimethyl-octanoate (200 mg, 254.87 μmol, 1.1 eq) in DMF (5 mL) was added KI (38.46 mg, 231.70 μmol, 1 eq) in sequence. Then the mixture was stirred at 60° C. for 12 hr. The reaction mixture diluted with by addition H₂O 50 mL, and then extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=10/1 to 1/1) to give compound 1-octylnonyl 8-[2-[4-[2-[[7,7-dimethyl-8-(1-octylnonoxy)-8-oxo-octyl]-(5,5-dimethyl-6-oxo-6-undecoxy-hexyl)amino]ethyl]piperazin-1-yl]ethyl-(5,5-dimethyl-6-oxo-6-unde coxy-hexyl)amino]-2,2-dimethyl-octanoate (39 mg, 120.05 μmol, 10.64% yield) as yellow oil.

¹H NMR (400 MHz, CDCl₃), 4.80-4.86 (m, 2H), 4.04 (t, J=6.4 Hz, 4H), 2.39-2.56 (m, 24H), 1.58-1.63 (m, 4H), 1.49-1.53 (m, 14H), 1.36-1.41 (m, 8H), 1.21-1.33 (m, 98H), 1.15 (s, 24H), 0.88 (t, J=6.4 Hz, 18H). LCMS (CAD): (½M+H⁺): 791.8 @ 13.372 min. LCMS (ELSD): (½M+H⁺): 791.9 @ 13.754 min.

### 9.8. Synthesis of 2424

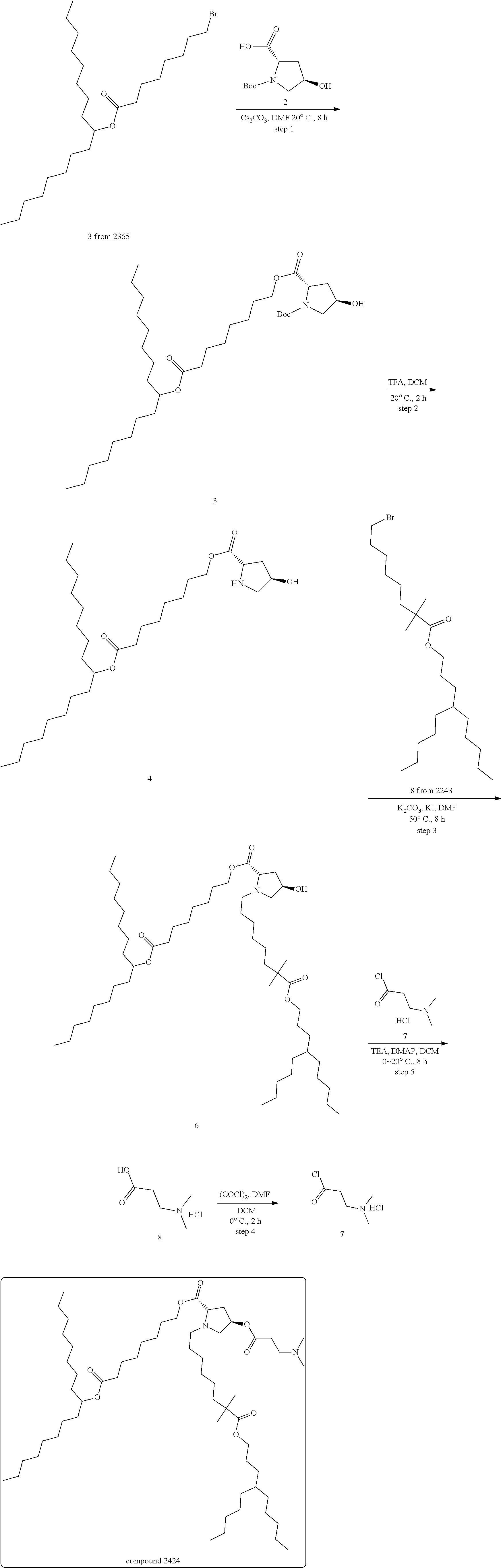

### Step 1:

To a solution of (2S,4R)-1-tert-butoxycarbonyl-4-hydroxy-pyrrolidine-2-carboxylic acid (15 g, 64.87 mmol, 1 eq) and 1-octylnonyl 8-bromooctanoate (35.93 g, 77.84 mmol, 1.2 eq) in DMF (200 mL) was added Cs₂CO₃ (46.50 g, 142.71 mmol, 2.2 eq). The mixture was stirred at 20° C. for 8 hr. The reaction mixture was quenched by addition H₂O 200 mL at 0° C., and then extracted with EtOAc 600 mL (200 mL*3). The combined organic layers were washed with sat, brine 400 mL (200 mL*2), dried over Na₂SO₄, filtered and concentrated under reduced pressure to give a residue. The residue was purified by column chromatography (SiO₂, Petroleum ether/Ethyl acetate=50/1 to 3/1) to give compound O1-tert-butyl O2-[8-(1-octylnonoxy)-8-oxo-octyl] (2S,4R)-4-hydroxypyrrolidine-1,2-dicarboxylate (30 g, 49.03 mmol, 75.58% yield) as colorless oil.

### Step 2:

To a solution of O1-tert-butyl O2-[8-(1-octylnonoxy)-8-oxo-octyl] (2S,4R)-4-hydroxypyrrolidine-1,2-dicarboxylate (15 g, 24.51 mmol, 1 eq) in DCM (200 mL) was added TFA (100 mL). The mixture was stirred at 20° C. for 2 hr. The mixture was concentrated under reduced pressure and adjust pH to 8 with sat. NaHCO₃, extracted with EtOAc 450 mL (150 mL*3). The combined organic layers were dried over Na₂SO₄, filtered and concentrated under reduced pressure to give a residue. The residue was purified by column chromatography (SiO₂, Petroleum ether/Ethyl acetate=5/1 to EtOAc/MeOH=3/1, added 0.5% NH₃·H₂O) to give compound [8-(1-octylnonoxy)-8-oxo-octyl] (2S,4R)-4-hydroxypyrrolidine-2-carboxylate (11 g, 21.49 mmol, 87.68% yield) as yellow oil.

¹H NMR (400) MHz, CDCl₃), 4.85-4.88 (m, 1H), 4.48-4.50 (m, 1H), 4.13-4.18 (m, 3H), 3.11-3.21 (m, 2H), 2.27-2.30 (m, 3H), 2.05-2.15 (m, 1H), 1.63-1.65 (m, 4H), 1.50-1.51 (m, 4H), 1.26-1.34 (m, 30H), 0.88 (t, J=6.4 Hz, 6H).

### Step 3:

To a solution of [8-(1-octylnonoxy)-8-oxo-octyl] (2S,4R)-4-hydroxypyrrolidine-2-carboxylate (5.5 g, 10.75 mmol, 1 eq) in DMF (60 mL) was added K₂CO₃ (4.46 g, 32.24 mmol, 3 eq), KI (892.01 mg, 5.37 mmol, 0.5 eq) and 4-pentylnonyl 8-bromo-2,2-dimethyl-octanoate (7.21 g, 16.12 mmol, 1.5 eq). The mixture was stirred at 50° C. for 8 hr. The reaction mixture was quenched by addition H₂O 60 mL at 0° C. and then extracted with EtOAc 150 mL (50 mL*3). The combined organic layers were washed with sat, brine 150 ml (50 mL*3), dried over Na₂SO₄, filtered and concentrated under reduced pressure to give a residue. The residue was purified by column chromatography (SiO₂, Petroleum ether/Ethyl acetate=10/1 to 0.1, added 0.5% NH₃·H₂O) to give compound [8-(1-octylnonoxy)-8-oxo-octyl] (2S,4R)-1-[7,7-dimethyl-8-oxo-8-(4-pentylnonoxy)octyl]-4-hydroxy-pyrrolidine-2-carboxylate (5.3 g, 6.03 mmol, 56.14% yield) as yellow oil.

¹H NMR (400 MHz, CDCl₃), 4.85-4.88 (m, 1H), 4.48 (s, 1H), 4.01-4.12 (m, 4H), 3.40-3.51 (m, 2H), 2.44-2.69 (m, 2H), 2.28-2.30 (m, 4H), 1.45-1.63 (m, 16H), 1.24-1.34 (m, 56H), 1.16 (s, 6H), 0.89 (t, J=6.8 Hz, 12H).

### Step 4:

To a solution of 3-(dimethylamino) propanoic acid (3 g, 19.53 mmol, 1 eq, HCl) in DCM (20 mL) was added DMF (71.38 mg, 976.52 μmol, 75.13 μL, 0.05 eq) and (COCl)₂ (2.97 g, 23.44 mmol, 2.05 mL, 1.2 eq). The mixture was stirred at 0° C. for 2 h. The mixture was concentrated under reduced pressure to give compound 3-(dimethylamino)propanoyl chloride (3.36 g, crude, HCl) as a white solid.

### Step 5:

To a solution of [8-(1-octylnonoxy)-8-oxo-octyl] (2S,4R)-1-[7,7-dimethyl-8-oxo-8-(4-pentylnonoxy)octyl]-4-hydroxy-pyrrolidine-2-carboxylate (3.4 g, 3.87 mmol, 1 eq) in DCM (30 mL) was added TEA (3.92 g, 38.71 mmol, 5.39 mL, 10 eq) and DMAP (236.44 mg, 1.94 mmol, 0.5 eq) and 3-(dimethylamino)propanoyl chloride (3.33 g, 19.35 mmol, 5 eq, HCl) at 0° C. The mixture was stirred at 20° C. for 8 hr. The reaction mixture was quenched by addition H₂O 50 mL at 0° C. and then extracted with EtOAc 90 mL (30 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1, added 0.5% NH₃·H₂O) to give compound [8-(1-octylnonoxy)-8-oxo-octyl] (2S,4R)-4-[3-(dimethylamino)propanoyloxy]-1-[7,7-dimethyl-8-oxo-8-(4-pentylnonoxy)octyl]pyrrolidine-2-carboxylate (1.08 g, 3.27 mmol, 84.57% yield) as colorless oil.

¹H NMR (400 MHz, CDCl₃), 5.25-5.28 (m, 1H), 4.85-4.88 (m, 1H), 4.01-4.13 (m, 4H), 3.43-3.55 (m, 2H), 2.07-2.68 (m, 18H), 1.59-1.63 (m, 6H), 1.47-1.51 (m, 8H), 1.24-1.34 (m, 56H), 1.15 (s, 6H), 0.89 (t, J=6.8 Hz, 12H).

LCMS: (M+H⁺): 977.8 @ 9.582 min. LCMS: (M+H⁺): 977.3 @ 11.443 min.

### 9.9. Synthesis of 2425

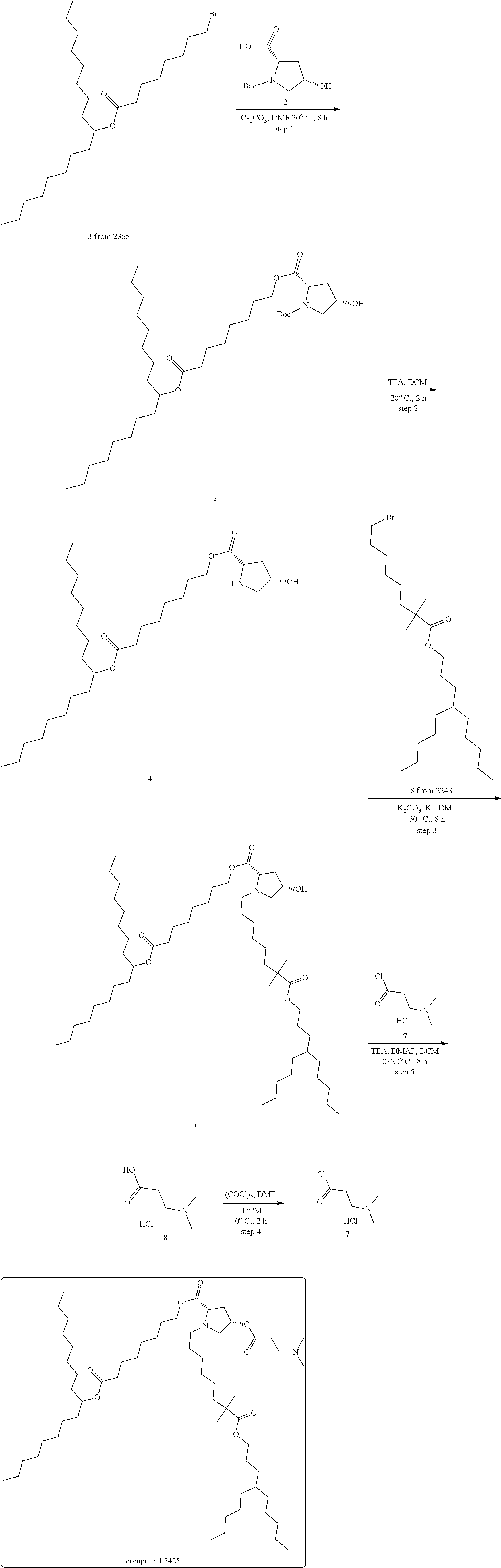

### Step 1:

To a solution of 1-octylnonyl 8-bromooctanoate (20 g, 43.33 mmol, 1 eq), (2S,4S)-1-tert-butoxycarbonyl-4-hydroxy-pyrrolidine-2-carboxylic acid (12.02 g, 52.00 mmol, 1.2 eq) in DMF (200 mL) was added Cs₂CO₃ (31.06 g, 95.33 mmol, 2.2 eq). The mixture was stirred at 20° C. for 8 hr. The reaction mixture was quenched by addition H₂O 200 mL at 0° C., and then extracted with EtOAc 600 mL (200 mL*3). The combined organic layers were washed with sat.brine 400 mL (200 mL*2), dried over Na₂SO₄, filtered and concentrated under reduced pressure to give a residue. The residue was purified by column chromatography (SiO₂, Petroleum ether/Ethyl acetate=50/1 to 3/1) to give compound O1-tert-butyl O2-[8-(1-octylnonoxy)-8-oxo-octyl] (2S,4S)-4-hydroxypyrrolidine-1,2-dicarboxylate (49 g, 80.08 mmol, 61.60% yield) as colorless oil.

¹H NMR (400 MHz, CDCl₃), 4.85-4.88 (m, 1H), 4.10-4.35 (m, 4H), 3.54-3.69 (m, 2H), 2.05-2.30 (m, 4H), 1.65-1.69 (m, 6H), 1.42-1.47 (m, 9H), 1.25-1.30 (m, 32H), 0.88 (t, J=6.8 Hz, 6H).

### Step 2:

To a solution of O1-tert-butyl O2-[8-(1-octylnonoxy)-8-oxo-octyl] (2S,4S)-4-hydroxypyrrolidine-1,2-dicarboxylate (10 g, 16.34 mmol, 1 eq) in DCM (100 mL) was added TFA (33 mL). The mixture was stirred at 20° C. for 2 hr. The mixture was concentrated under reduced pressure and adjust pH to 8 with sat. NaHCO₃, extracted with EtOAc 450 mL (150 mL*3). The combined organic layers were dried over Na₂SO₄, filtered and concentrated under reduced pressure to give a residue. The residue was purified by column chromatography (SiO₂, Petroleum ether/Ethyl acetate=5/1 to EtOAc/MeOH=3/1, added 0.5% NH₃·H₂O) to give compound [8-(1-octylnonoxy)-8-oxo-octyl] (2S,4S)-4-hydroxypyrrolidine-2-carboxylate (7 g, 13.68 mmol, 83.69% yield) as yellow oil.

### Step 3:

To a solution of [8-(1-octylnonoxy)-8-oxo-octyl] (2S,4S)-4-hydroxypyrrolidine-2-carboxylate (7 g, 13.68 mmol, 1 eq) in DMF (60 mL) was added K₂CO₃ (5.67 g, 41.03 mmol, 3 eq), KI (1.14 g, 6.84 mmol, 0.5 eq) and 4-pentylnonyl 8-bromo-2,2-dimethyl-octanoate (9.18 g, 20.52 mmol, 1.5 eq). The mixture was stirred at 50° C. for 8 hr. The reaction mixture was quenched by addition H₂O 60 mL at 0° C. and then extracted with EtOAc 150 mL (50 mL*3). The combined organic layers were washed with sat, brine 150 mL (50 mL*3), dried over Na₂SO₄, filtered and concentrated under reduced pressure to give a residue. The residue was purified by column chromatography (SiO₂, Petroleum ether/Ethyl acetate=10/1 to 9/1) to give compound [8-(1-octylnonoxy)-8-oxo-octyl] (2S,4S)-1-[7,7-dimethyl-8-oxo-8-(4-pentylnonoxy)octyl]-4-hydroxy-pyrrolidine-2-carboxylate (6 g, 6.83 mmol, 33.29% yield, 100% purity, 2 batches) as yellow oil.

¹H NMR (400 MHz, CDCl₃), 4.86-4.89 (m, 1H), 4.02-4.27 (m, 5H), 3.05-3.26 (m, 3H), 2.29-2.63 (m, 6H), 1.82-1.95 (m, 1H), 1.16-1.64 (m, 74H), 0.89 (t, J=7.2 Hz, 12H).

### Step 4:

To a solution of 3-(dimethylamino) propanoic acid (4 g, 26.04 mmol, 1 eq, HCl) in DCM (20 mL) was added oxalyl dichloride (16.53 g, 130.20 mmol, 11.40 mL, 5 eq) and DMF (71.38 mg, 976.52 μmol, 75.13 μL, 0.05 eq). The mixture was stirred at 0° C. for 2 h. The mixture was concentrated under reduced pressure to give compound 3-(dimethylamino)propanoyl chloride (3.36 g, crude, HCl) as a white solid.

### Step 5:

To a solution of [8-(1-octylnonoxy)-8-oxo-octyl] (2S,4S)-1-[7,7-dimethyl-8-oxo-8-(4-pentylnonoxy)octyl]-4-hydroxy-pyrrolidine-2-carboxylate (6 g, 6.83 mmol, 1 eq) in DCM (60 mL) was added TEA (6.22 g, 61.48 mmol, 8.56 mL, 9 eq) and DMAP (83.45 mg, 683.06 μmol, 0.1 eq) and 3-(dimethylamino)propanoyl chloride (3.53 g, 20.49 mmol, 3 eq, HCl) at 0° C. The mixture was stirred at 20° C. for 8 hr. The reaction mixture was quenched by addition H₂O 50 mL at 0° C., and then extracted with EtOAc 300 mL (100 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1, added 0.5% NH₃·H₂O) and p-HPLC (column: Welch Xtimate C1 100*30 mm*5 μm; mobile phase: [H₂O (0.05% HCl)-ACN]; gradient: 35%-70% B over 20.0 min). The mixture was adjusted pH-7 with sat. NaHCO₃ aq, and extracted with EtOAc 150 mL (50 mL*3). The combined organic layers were dried over Na₂SO₄, filtered and concentrated under reduced pressure to give compound [8-(1-octylnonoxy)-8-oxo-octyl] (2S,4S)-4-[3-(dimethylamino)propanoyloxy]-1-[7,7-dimethyl-8-oxo-8-(4-pentylnonoxy)octyl]pyrrolidine-2-carboxylate (1.7 g, 1.74 mmol, 32.12% yield, 100% purity) as colorless oil.

¹H NMR (400 MHz, CDCl₃), 5.20-5.22 (m, 1H), 4.85-4.89 (m, 1H), 4.01-4.15 (m, 4H), 3.24-3.27 (m, 1H), 3.10-3.12 (m, 1H), 2.74-2.76 (m, 1H), 2.60-2.65 (m, 4H), 2.49-2.51 (m, 2H), 2.23-2.26 (m, 9H), 1.98-2.15 (m, 1H), 1.61-1.66 (m, 7H), 1.50-1.52 (m, 4H), 1.26-1.34 (m, 58H), 1.16 (s, 6H), 0.88 (t, J=7.2 Hz, 12H).

LCMS: (M+H⁺): 977.9 @ 9.771 min.

### 9.10. Synthesis of 2432

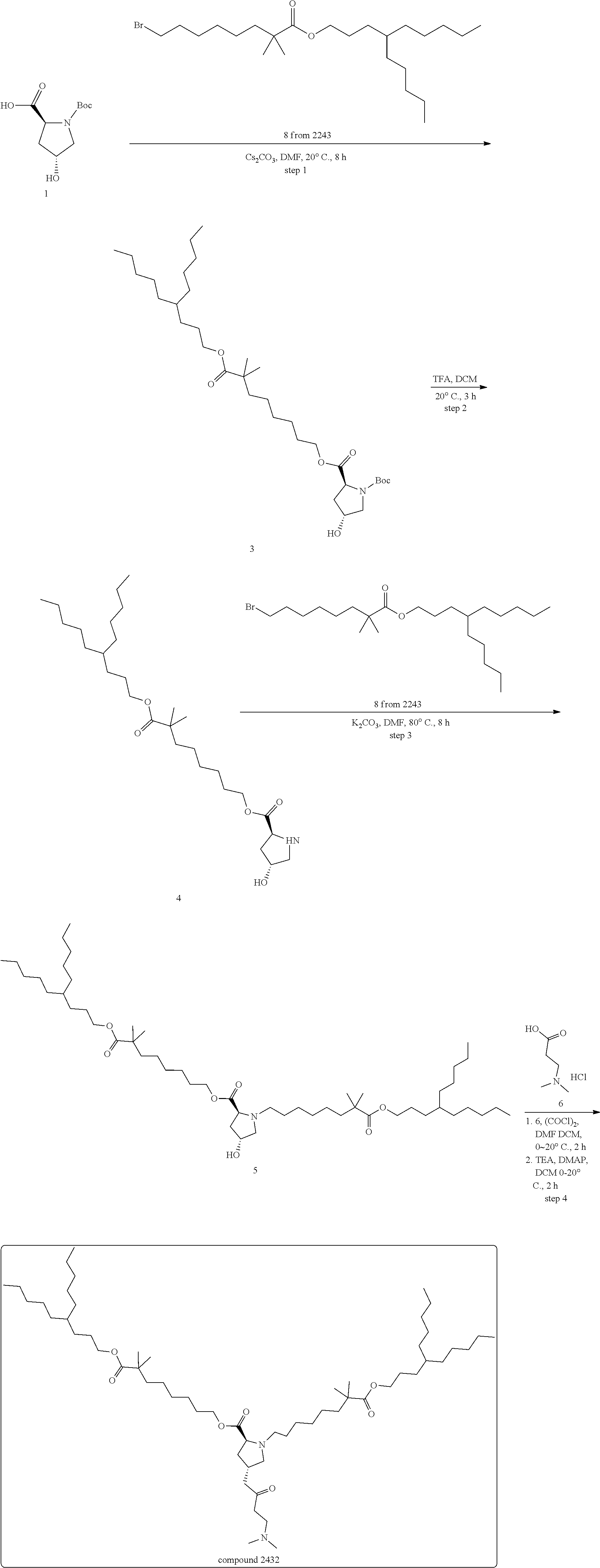

### Step 1:

A mixture of 4-pentylnonyl 8-bromo-2,2-dimethyl-octanoate (6.97 g, 15.57 mmol, 1.2 eq), (2S,4R)-1-tert-butoxycarbonyl-4-hydroxy-pyrrolidine-2-carboxylic acid (3 g, 12.97 mmol, 1 eq) and Cs₂CO₃ (9.30 g, 28.54 mmol, 2.2 eq) in DMF (60 mL) was stirred at 20° C. for 8 hr under N₂ atmosphere. The combined organic phase was diluted with EtOAc 100 mL and washed with water 200 mL (100 mL*2) and brine 200 mL (100 mL*2), dried with anhydrous Na₂SO₄, filtered and concentrated under reduced pressure to give a residue. The residue was purified by column chromatography (SiO₂, Petroleum ether/Ethyl acetate/NH₃·H₂O=10/1/0 to 1/1/0.1) to give a compound O1-tert-butyl O2-[7,7-dimethyl-8-oxo-8-(4-pentylnonoxy)octyl] (2S,4R)-4-hydroxypyrrolidine-1,2-dicarboxylate (6 g, 9.84 mmol, 75.81% yield) as colorless oil.

### Step 2:

To a solution of O1-tert-butyl O2-[7,7-dimethyl-8-oxo-8-(4-pentylnonoxy)octyl] (2S,4R)-4-hydroxypyrrolidine-1,2-dicarboxylate (2 g, 3.35 mmol, 1 eq) in DCM (12 mL) was added TFA (6.14 g, 53.85 mmol, 4 mL, 16.10 eq). The mixture was stirred at 20° C. for 3 hr. The reaction mixture was adjusted pH-7 with saturated NaHCO₃ aqueous and extracted with EtOAc 60 mL (20 mL*3), dried over Na₂SO₄, filtered and concentrated under reduced pressure to give a residue. The residue was purified by column chromatography (SiO₂, Petroleum ether/Ethyl acetate=5/1 to 0/1) to give a compound [7,7-dimethyl-8-oxo-8-(4-pentylnonoxy)octyl] (2S,4R)-4-hydroxypyrrolidine-2-carboxylate (1.1 g, 2.21 mmol, 66.06% yield) as yellow oil.

¹H NMR (400 MHz, CDCl₃), 4.44-4.45 (m, 1H), 4.10-4.14 (m, 4H), 2.85-3.19 (m, 2H), 2.05-2.07 (m, 5H), 1.49-1.64 (m, 5H), 1.27-1.31 (m, 26H), 1.17 (s, 6H), 0.89 (t, J=6.8 Hz, 6H).

### Step 3:

To a solution of [7,7-dimethyl-8-oxo-8-(4-pentylnonoxy)octyl] (2S,4R)-4-hydroxypyrrolidine-2-carboxylate (1.1 g, 2.21 mmol, 1 eq), 4-pentylnonyl 8-bromo-2,2-dimethyl-octanoate (1.19 g, 2.65 mmol, 1.2 eq) in DMF (20 mL) was added K₂CO₃ (916.28 mg, 6.63 mmol, 3 eq). The mixture was stirred at 80° C. for 8 hr. The reaction mixture was diluted with H₂O 50 mL and extracted with EtOAc 120 mL (40 mL*3). The combined organic layers were dried over Na₂SO₄, filtered and concentrated under reduced pressure to give a residue. The residue was purified by column chromatography (SiO₂, Petroleum ether/Ethyl acetate/NH₃·H₂O=10/1/1 to 1/1/0.5) to give a compound [7,7-dimethyl-8-oxo-8-(4-pentylnonoxy)octyl] (2S,4R)-1-[7,7-dimethyl-8-oxo-8-(4-pentylnonoxy)octyl]-4-hydroxy-pyrrolidine-2-carboxylate (1.2 g, 1.19 mmol, 54.03% yield) as yellow oil.

### Step 4:

A mixture of 3-(dimethylamino) propanoic acid (0.6 g, 3.91 mmol, 1 eq, HCl) in DCM (5 mL) was added (COCl)₂ (2.48 g, 19.53 mmol, 1.71 mL, 5 eq). DMF (28.55 mg, 390.61 μmol, 30.05 μL, 0.1 eq) at 0° C. The mixture was stirred at 20° C. for 2 hr under N₂ atmosphere. The reaction mixture was concentrated under reduced pressure to give a compound 3-(dimethylamino)propanoyl chloride (0.6 g, crude, HCl) as yellow oil without purification.

Then the crude 3-(dimethylamino)propanoyl chloride (497.63 mg, 2.89 mmol, 5 eq, HCl) was added to a solution of [7,7-dimethyl-8-oxo-8-(4-pentylnonoxy)octyl] (2S,4R)-1-[7,7-dimethyl-8-oxo-8-(4-pentylnonoxy)octyl]-4-hydroxy-pyrrolidine-2-carboxylate (0.5 g, 578.46 μmol, 1 eq), TEA (351.20 mg, 3.47 mmol, 483.08 μL, 6 eq). DMAP (14.13 mg, 115.69 μmol, 0.2 eq) in DCM (5 mL) at 0° C. The mixture was stirred at 20° C. for 2 hr. The reaction mixture was diluted with H₂O 20 mL and extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=1/0 to 1/0) and p-HPLC (column: Xselect CSH C18 100*30 mm*5 μm; mobile phase: [H₂O (0.05% HCl)-ACN]; gradient: 50%-95% B over 12.0 min) to give a compound [7,7-dimethyl-8-oxo-8-(4-pentylnonoxy)octyl] (2S,4R)-4-[3-(dimethylamino)propanoyloxy]-1-[7,7-dimethyl-8-oxo-8-(4-pentylnonoxy)octyl]pyrrolidine-2-carboxylate (40 mg, 41.52 μmol, 13.50% yield, 98% purity, HCl) as yellow oil.

¹H NMR (400 MHz, CDCl₃), 12.70 (brs, 2H), 5.37 (brs, 1H), 4.01-4.53 (m, 7H), 2.87-3.60 (m, 15H), 1.15-1.61 (m, 75H), 0.89 (t, J=7.2 Hz, 12H).

LCMS: (M+H⁺): 963.8 @ 11.363 min.

### 9.11. Synthesis of 2433

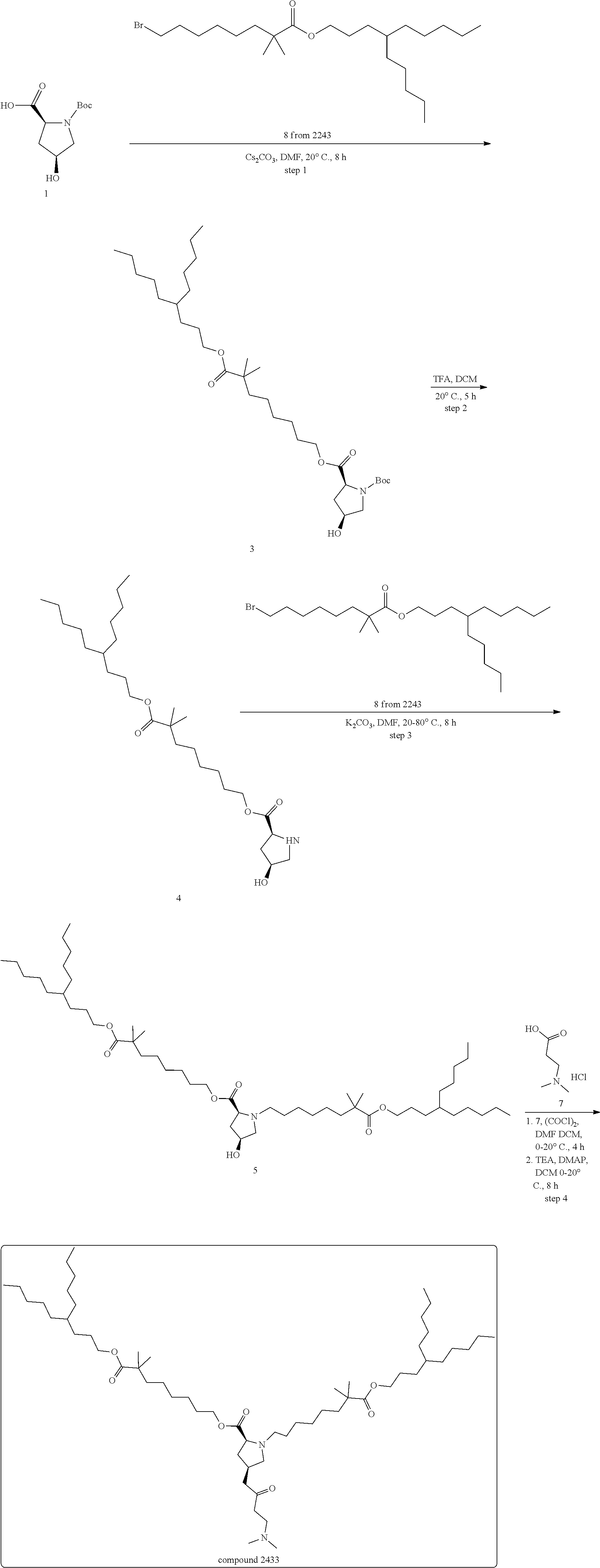

### Step 1:

To a solution of 4-pentylnonyl 8-bromo-2,2-dimethyl-octanoate (4 g, 8.94 mmol, 1.2 eq) and (2S,4S)-1-tert-butoxycarbonyl-4-hydroxy-pyrrolidine-2-carboxylic acid (1.72 g, 7.45 mmol, 1 eq) in DMF (100 mL) was added Cs₂CO₃ (5.34 g, 16.39 mmol, 2.2 eq) at 20° C. The mixture was degassed and purged with N₂ for 3 times, and then stirred at 20° C. for 8 hr under N₂ atmosphere. The reaction mixture was filtered and the filtrate was concentrated under reduced pressure to give a residue. The residue was diluted with EtOAc 100 mL and washed with brine 90 mL (30 mL*3), dried with anhydrous Na₂SO₄, filtered and concentrated under reduced pressure to give a residue. The residue was purified by column chromatography (SiO₂, Petroleum ether/Ethyl acetate=8/1 to 0/1, 5% NH₃·H₂O) to give compound O1-tert-butyl O2-[7,7-dimethyl-8-oxo-8-(4-pentylnonoxy)octyl] (2S,4S)-4-hydroxypyrrolidine-1,2-dicarboxylate (2 g, 3.35 mmol, 44.91% yield) as colourless oil.

¹H NMR (400 MHz, CDCl₃), 4.10-4.40 (m, 4H), 4.03 (t, J=6.8 Hz, 2H), 3.25-3.80 (m, 3H), 2.25-2.45 (m, 1H), 2.05-2.11 (m, 1H), 1.60-1.68 (m, 4H), 1.42-1.53 (m, 11H), 1.24-1.46 (m, 25H), 1.16 (s, 6H), 0.89 (1, J=6.8 Hz, 6H).

### Step 2:

To a solution of O1-tert-butyl O2-[7,7-dimethyl-8-oxo-8-(4-pentylnonoxy)octyl] (2S,4S)-4-hydroxypyrrolidine-1,2-dicarboxylate (1.00 g, 1.67 mmol, 1 eq) in DCM (14 mL) was added TFA (10.75 g, 94.24 mmol, 7 mL, 1 eq). The mixture was stirred at 20° C. for 5 hr. The reaction mixture was concentrated under reduced pressure to get a residue. The reaction mixture was adjusted to pH=7.0 with sat. NaHCO₃ aq, and extracted with EtOAc 50 mL (25 mL*2). The combined organic layers were dried over Na₂SO₄, filtered and concentrated under reduced pressure to give compound [7,7-dimethyl-8-oxo-8-(4-pentylnonoxy)octyl] (2S,4S)-4-hydroxypyrrolidine-2-carboxylate (1.8 g, crude) as yellow oil.

### Step 3:

To a solution of [7,7-dimethyl-8-oxo-8-(4-pentylnonoxy)octyl] (2S,4S)-4-hydroxypyrrolidine-2-carboxylate (1.8 g, 3.62 mmol, 1 eq) and 4-pentylnonyl 8-bromo-2,2-dimethyl-octanoate (1.94 g, 4.34 mmol, 1.2 eq) in DMF (30 mL) was added K₂CO₃ (1.50 g, 10.85 mmol, 3 eq) at 20° C. The mixture was degassed and purged with N₂ for 3 times, and then stirred at 80° C. for 8 hr under N₂ atmosphere. The reaction mixture was filtered and the filtrate was concentrated under reduced pressure to give a residue. The residue was diluted with EtOAc 100 mL and washed with brine 90 mL (30 mL*3), dried with anhydrous Na₂SO₄, filtered and concentrated under reduced pressure to give a residue. The residue was purified by column chromatography (SiO₂, Petroleum ether/Ethyl acetate=8/1 to 0/1, 5% NH; H₂O) and prep-HPLC (column: Xselect CSH C18 100*30 mm*5 μm; mobile phase: [H₂O (0.1% TFA)-THF:ACN=1:3]; gradient: 45%-85% B over 8.0 min). The residue was concentrated under reduced pressure to get a residue. The residue was adjusted pH=7 with sat. NaHCO₃ aq, and extracted with EtOAc 300 mL (100 mL*3). The combined organic layers were dried over Na₂SO₄, filtered and concentrated under reduced pressure to give compound [7,7-dimethyl-8-oxo-8-(4-pentylnonoxy)octyl] (2S,4S)-1-[7,7-dimethyl-8-oxo-8-(4-pentylnonoxy)octyl]-4-hydroxy-pyrrolidine-2-carboxylate (0.356 g, 411.86 μmol, 11.37% yield) as yellow oil.

¹H NMR (400 MHz, CDCl₃), 4.34 (s, 1H), 4.05-4.18 (m, 2H), 3.95-4.05 (m, 4H), 3.25-3.29 (m, 2H), 2.60-2.63 (m, 2H), 2.47-2.54 (m, 1H), 2.27-2.42 (m, 1H), 1.92-2.02 (m, 1H), 1.51-1.67 (m, 6H), 1.31-1.51 (m, 6H), 1.24-1.30 (m, 50H), 1.15-1.17 (m, 12H), 0.89 (t, J=6.8 Hz, 12H).

### Step 4:

To a solution of 3-(dimethylamino) propanoic acid (0.3 g, 1.95 mmol, 1 eq, HCl) in DCM (8 mL) was added oxalyl dichloride (1.24 g, 9.77 mmol, 854.80 μL, 5 eq) and DMF (43.85 mg, 599.86 μmol, 46.15 μL, 3.07e-1 eq) at 0° C. The mixture was degassed and purged with N₂ for 3 times, and then stirred at 20° C. for 4 hr under N₂ atmosphere. The reaction mixture was concentrated under reduced pressure to give compound 3-(dimethylamino)propanoyl chloride (0.2 g, crude, HCl) as colourless oil. Then the crude 3-(dimethylamino)propanoyl chloride (199.05 mg, 1.16 mmol, 5 eq, HCl) in DCM (3 mL) was dropwise added to a solution of [7,7-dimethyl-8-oxo-8-(4-pentylnonoxy)octyl] (2S,4S)-1-[7,7-dimethyl-8-oxo-8-(4-pentylnonoxy)octyl]-4-hydroxy-pyrrolidine-2-carboxylate (0.2 g, 231.38 μmol, 1 eq) and TEA (234.13 mg, 2.31 mmol, 322.06 μL, 10 eq) and DMAP (5.65 mg, 46.28 μmol, 0.2 eq) in DCM (7 mL) at 0° C. The mixture was degassed and purged with N₂ for 3 times, and then stirred at 20° C. for 8 hr under N₂ atmosphere. The reaction mixture was diluted with H₂O 50 mL and extracted with EtOAc 100 mL (25 mL*4). The combined organic layers were dried over Na₂SO₄, filtered and concentrated under reduced pressure to give a residue. The residue was purified by column chromatography (SiO₂, Petroleum ether/Ethyl acetate=8/1 to 0.1, 5% NH₃·H₂O) and prep-HPLC (column: Xselect CSH C18 100*30 mm*5 μm; mobile phase: [H₂O (0.1% TFA)-THF:ACN=1:3]; gradient: 35%-75% B over 10.0 min). The solution was concentrated under reduced pressure to give compound [7,7-dimethyl-8-oxo-8-(4-pentylnonoxy)octyl] (2S,4S)-4-[3-(dimethylamino)propanoyloxy]-1-[7,7-dimethyl-8-oxo-8-(4-pentylnonoxy)octyl]pyrrolidine-2-carboxylate (0.022 g, 17.88 μmol, 9.86% yield, 97% purity, TFA) as yellow oil.

¹H NMR (400 MHz, CDCl₃), 13.45 (s, 1H), 5.31 (s, 1H), 4.21 (t, J=7.6 Hz, 2H), 4.01-4.05 (m, 4H), 3.87-3.89 (m, 2H), 3.32-3.39 (m, 2H), 3.19-3.22 (m, 1H), 2.76-2.90 (m, 11H), 2.41-2.47 (m, 1H), 1.6-1.69 (m, 4H), 1.57-1.59 (m, 4H), 1.46-1.54 (m, 4H), 1.24-1.47 (m, 50H), 1.15-1.17 (m, 12H), 0.89 (1, J=7.8 Hz, 12H). LCMS: (½M+H⁺): 963.7 @ 10.929 min.

### 9.12. Synthesis of 2441

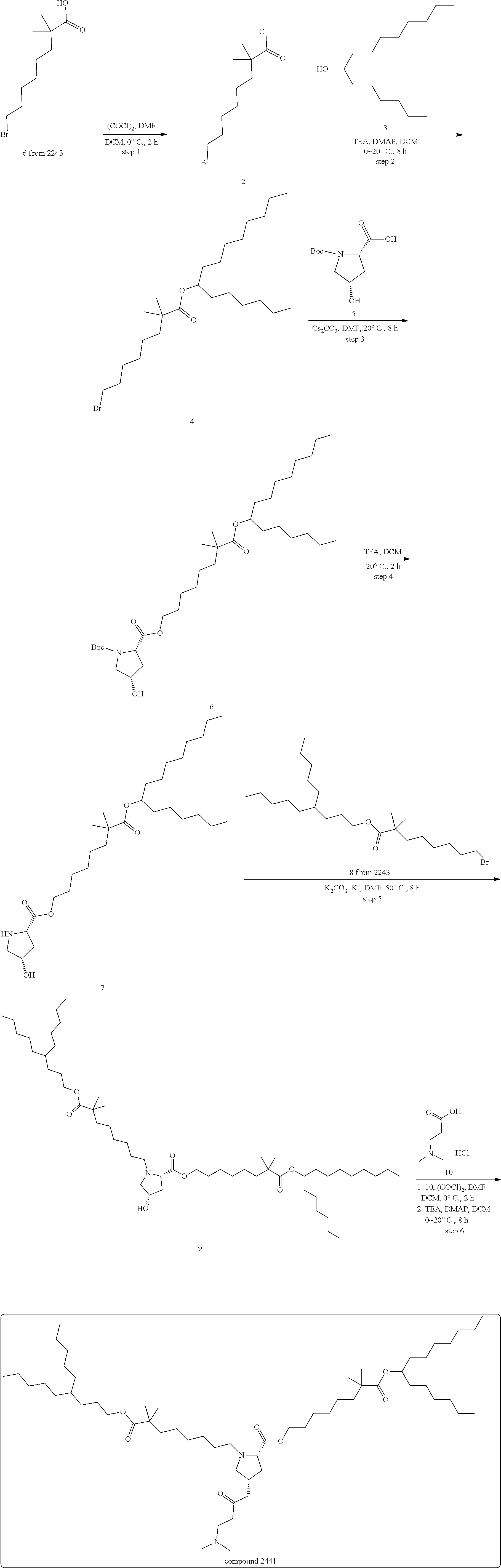

### Step 1:

To a solution of 8-bromo-2,2-dimethyl-octanoic acid (5 g, 19.91 mmol, 1 eq) in DCM (50 mL) was added DMF (72.76 mg, 995.38 μmol, 76.59 μL, 0.05 eq) and oxalyl dichloride (3.03 g, 23.89 mmol, 2.09 mL, 1.2 eq). The mixture was stirred at 0° C. for 2 hr. The mixture was concentrated under reduced pressure to give compound 8-bromo-2,2-dimethyl-octanoyl chloride (5.37 g, crude) as yellow oil.

### Step 2:

To a solution of pentadecan-7-ol (4.1 g, 17.95 mmol, 1 eq) in DCM (40 mL) was added TEA (5.45 g, 53.85 mmol, 7.50 mL, 3 eq), DMAP (1.10 g, 8.97 mmol, 0.5 eq) and 8-bromo-2,2-dimethyl-octanoyl chloride (5.32 g, 19.74 mmol, 1.1 eq) at 0° C. The mixture was stirred at 20° C. for 8 hr. The reaction mixture was quenched by addition H₂O 40 mL, and then extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=20/1 to 0/1) to give compound 1-hexylnonyl 8-bromo-2,2-dimethyl-octanoate (6.7 g, 14.52 mmol, 80.87% yield) as yellow oil.

¹H NMR (400 MHz, CDCl₃), 4.81-4.87 (m, 1H), 3.38-3.66 (m, 2H), 1.84-1.88 (m, 1H), 1.43-1.53 (m, 8H), 1.17-1.26 (m, 26H), 1.15 (s, 6H), 0.88 (t, J=6.4 Hz, 6H).

### Step 3:

To a solution of l-hexylnonyl 8-bromo-2,2-dimethyl-octanoate (6.47 g, 14.01 mmol, 1.2 eq) in DMF (70 mL) was added Cs₂CO₃ (8.37 g, 25.69 mmol, 2.2 eq) and (2S,4S)-1-tert-butoxy carbonyl-4-hydroxy-pyrrolidine-2-carboxylic acid (2.7 g, 11.68 mmol, 1 eq). The mixture was stirred at 20° C. for 8 hr. The reaction mixture was quenched by addition H₂O 60 mL, and then extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=1/0 to 0/1) to give compound O1-tert-butyl O2-[8-(1-hexylnonoxy)-7,7-dimethyl-8-oxo-octyl] (2S,4S)-4-hydroxypyrrolidine-1,2-dicarboxylate (4.4 g, 7.19 mmol, 61.59% yield) as yellow oil.

### Step 4:

To a solution of O1-tert-butyl O2-[8-(1-hexylnonoxy)-7,7-dimethyl-8-oxo-octyl] (2S,4S)-4-hydroxypyrrolidine-1,2-dicarboxylate (4.3 g, 7.03 mmol, 1 eq) in DCM (30 mL) was added TFA (15 mL). The mixture was stirred at 20° C. for 2 hr. The mixture was concentrated under reduced pressure, then adjust pH to 8 with sat. NaHCO₃, extracted with EtOAc 120 mL (40 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound [8-(1-hexylnonoxy)-7,7-dimethyl-8-oxo-octyl] (2S,4S)-4-hydroxypyrrolidine-2-carboxylate (2.5 g, 4.88 mmol, 69.51% yield) as yellow oil.

¹H NMR (400 MHz, CDCl₃), 4.82-4.83 (m, 1H), 4.43 (brs, 1H), 4.04-4.18 (m, 4H), 2.82-2.95 (m, 6H), 2.17-2.37 (m, 2H), 1.51-1.66 (m, 2H), 1.47-1.49 (m, 6H), 1.23-1.30 (m, 24H), 1.14 (s, 6H), 0.87 (t, J=6.8 Hz, 6H).

### Step 5:

To a solution of [8-(1-hexylnonoxy)-7,7-dimethyl-8-oxo-octyl] (2S,4S)-4-hydroxypyrrolidine-2-carboxylate (2.5 g, 4.88 mmol, 1 eq) in DMF (30 mL) was added K₂CO₃ (2.03 g, 14.65 mmol, 3 eq), KI (810.90 mg, 4.88 mmol, 1 eq) and 4-pentylnonyl 8-bromo-2,2-dimethyl-octanoate (6.56 g, 14.65 mmol, 3 eq). The mixture was stirred at 50° C. for 8 hr. The reaction mixture was quenched by addition H₂O 60 mL, and then extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=50/1 to 0/1) to give compound [8-(1-hexylnonoxy)-7,7-dimethyl-8-oxo-octyl] (2S,4S)-1-[7,7-dimethyl-8-oxo-8-(4-pentylnonoxy)octyl]-4-hydroxy-pyrrolidine-2-carboxylate (3 g, 3.42 mmol, 69.92% yield) as yellow oil.

¹H NMR (400 MHz, CDCl₃), 4.83-4.86 (m, 1H), 4.26-4.28 (m, 1H), 4.01-4.13 (m, 4H), 3.62-3.66 (m, 2H), 3.04-3.30 (m, 2H), 2.30-2.63 (m, 2H), 1.89-1.93 (m, 1H), 1.49-1.62 (m, 14H), 1.24-1.29 (m, 52H), 1.15 (s, 12H), 0.86-0.90 (m, 12H).

### Step 6:

To a solution of 3-(dimethylamino) propanoic acid (450 mg, 2.93 mmol, 1 eq, HCl) in DCM (10 mL) was added DMF (10.71 mg, 146.48 μmol, 11.27 μL, 0.05 eq) and oxalyl dichloride (446.21 mg, 3.52 mmol, 307.73 μL, 1.2 eq). The mixture was stirred at 0° C. for 2 hr. The mixture was concentrated under reduced pressure to give compound 3-(dimethylamino)propanoyl chloride (504 mg, crude, HCl) as yellow oil. To a solution of [8-(1-hexylnonoxy)-7,7-dimethyl-8-oxo-octyl] (2S,4S)-1-[7,7-dimethyl-8-oxo-8-(4-pentylnonoxy)octyl]-4-hydroxy-pyrrolidine-2-carboxylate (500 mg, 569.22 μmol, 1 eq) in DCM (10 mL) was added TEA (575.99 mg, 5.69 mmol, 792.28 μL, 10 eq), DMAP (34.77 mg, 284.61 μmol, 0.5 eq) and 3-(dimethylamino)propanoyl chloride (489.68 mg, 2.85 mmol, 5 eq, HCl) at 0° C. The mixture was stirred at 20° C. for 8 hr. The reaction mixture was quenched by addition H₂O 20 mL, and then extracted with EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound [8-(1-hexylnonoxy)-7,7-dimethyl-8-oxo-octyl] (2S,4S)-4-[3-(dimethylamino)propanoyl oxy]-1-[7,7-dimethyl-8-oxo-8-(4-pentylnonoxy)octyl]pyrrolidine-2-carboxylate (55 mg, 306.90 μmol, 9.89% yield, HCl salt) as yellow oil.

¹H NMR (400 MHz, CDCl₃), 11.33-13.40 (m, 2H), 5.37-5.48 (m, 1H), 4.79-4.85 (m, 1H), 4.23-4.52 (m, 4H), 4.01 (t, J=6.8 Hz, 2H), 3.10-3.64 (m, 7H), 2.83-2.96 (m, 7H), 2.57-2.60 (m, 1H), 1.68-1.70 (m, 2H), 1.45-1.59 (m, 11H), 1.23-1.29 (m, 52H), 1.15 (d, J=4.4 Hz, 12H), 0.86-0.90 (m, 12H). LCMS (CAD): (M+H⁺): 977.7 @ 11.103 min. LCMS (ELSD): (M+H⁺): 977.8 @ 9.624 min.

### 9.13. Synthesis of 2442

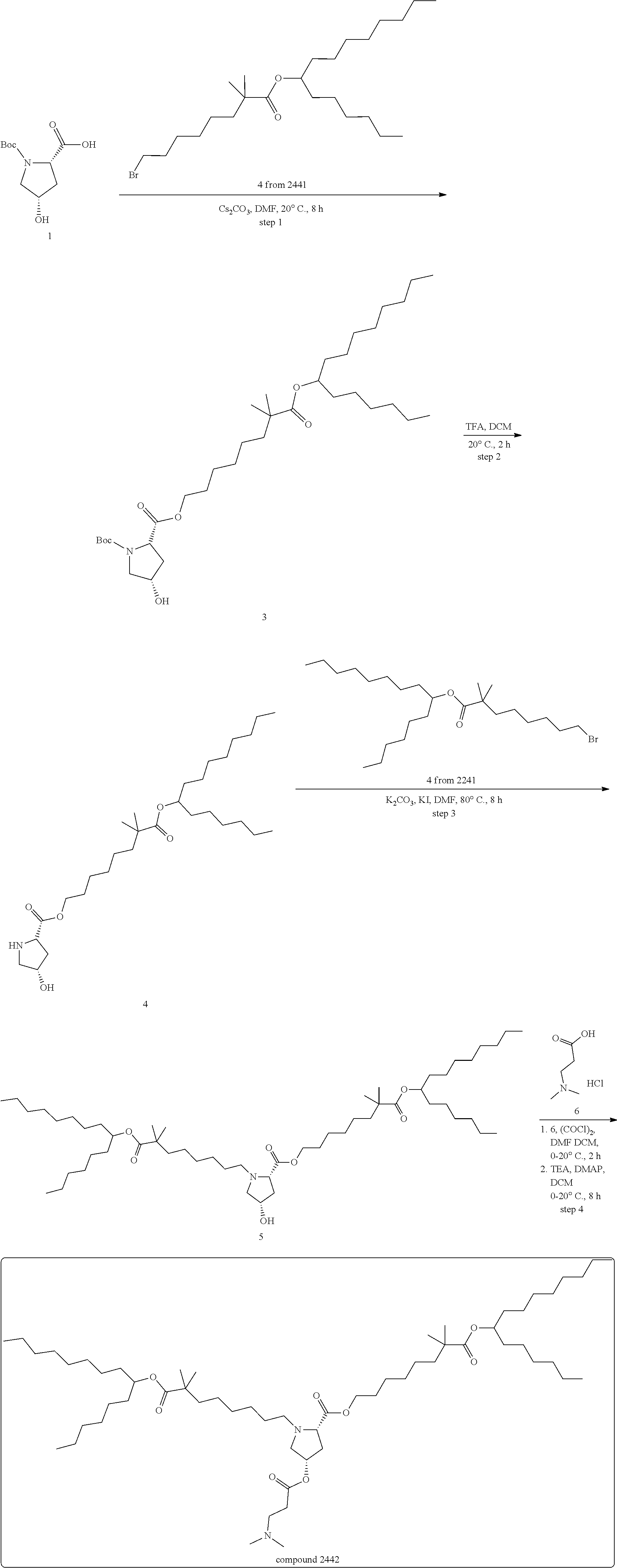

### Step 1:

A mixture of (2S,4S)-1-tert-butoxycarbonyl-4-hydroxy-pyrrolidine-2-carboxylic acid (2 g, 8.65 mmol, 1 eq), 1-hexylnonyl 8-bromo-2,2-dimethyl-octanoate (4.79 g, 10.38 mmol, 1.2 eq), Cs₂CO₃ (6.20 g, 19.03 mmol, 2.2 eq) in DMF (40 mL) was stirred at 20° C. for 8 hr under N₂ atmosphere. The combined organic phase was diluted with EtOAc 60 mL and washed with water 180 mL (60 mL*3) and brine 120 mL (60 mL*2), dried with anhydrous Na₂SO₄, filtered and concentrated under reduced pressure to give a residue. The residue was purified by column chromatography (SiO₂, Petroleum ether/Ethyl acetate/NH₃·H₂O=10/1/0 to 5/1/0.1) to give compound O1-tert-butyl O2-[8-(1-hexylnonoxy)-7,7-dimethyl-8-oxo-octyl] (2S,4S)-4-hydroxypyrrolidine-1,2-dicarboxylate (3 g, 4.90 mmol, 56.69% yield) as yellow oil.

¹H NMR (400 MHz, CDCl₃), 4.76-4.93 (m, 1H), 4.08-4.38 (m, 4H), 3.45-3.55 (m, 2H), 2.35-2.43 (m, 1H), 2.05-2.16 (m, 1H), 1.48-1.72 (m, 9H), 1.18-1.47 (m, 34H), 1.08-1.17 (m, 6H), 0.88 (t, J=6.4 Hz, 6H).

### Step 2:

To a solution of O1-tert-butyl O2-[8-(1-hexylnonoxy)-7,7-dimethyl-8-oxo-octyl] (2S,4S)-4-hydroxypyrrolidine-1,2-dicarboxylate (3 g, 4.90 mmol, 1 eq) in DCM (27 mL) was added TFA (13.82 g, 121.16 mmol, 9 mL, 24.71 eq). The mixture was stirred at 20° C. for 2 hr. The reaction mixture was adjusted pH=7 with saturated NaHCO₃ aqueous and extracted with EtOAc 150 mL (50 mL*3), dried over Na₂SO₄, filtered and concentrated under reduced pressure to give a residue. The residue was purified by column chromatography (SiO₂, Petroleum ether/Ethyl acetate=10/1 to 1/1, 3% NH₃·H₂O) to give compound [8-(1-hexylnonoxy)-7,7-dimethyl-8-oxo-octyl] (2S,4S)-4-hydroxypyrrolidine-2-carboxylate (1.9 g, 3.71 mmol, 75.72% yield) as yellow oil.

### Step 3:

A mixture of [8-(1-hexylnonoxy)-7,7-dimethyl-8-oxo-octyl] (2S,4S)-4-hydroxypyrrolidine-2-carboxylate (1 g, 1.95 mmol, 1 eq), 1-hexylnonyl 8-bromo-2,2-dimethyl-octanoate (1.08 g, 2.34 mmol, 1.2 eq). K₂CO₃ (810.15 mg, 5.86 mmol, 3 eq), KI (162.18 mg, 976.99 μmol, 0.5 eq) in DMF (10 mL) was degassed and purged with N₂ for 3 times, and then the mixture was stirred at 80° C. for 8 hr under N₂ atmosphere. The combined organic phase was diluted with EtOAc 40 mL and washed with water 60 mL (20 mL*3) and brine 40 mL (20 mL*2), dried with anhydrous Na₂SO₄, filtered and concentrated under reduced pressure to give a residue. The residue was purified by column chromatography (SiO₂, Petroleum ether/Ethyl acetate=1/0 to 5/1.3% NH₃·H₂O) to give compound [8-(1-hexylnonoxy)-7,7-dimethyl-8-oxo-octyl] (2S,4S)-1-[8-(1-hexylnonoxy)-7,7-dimethyl-8-oxo-octyl]-4-hydroxy-pyrrolidine-2-carboxylate (0.86 g, 905.85 μmol, 46.36% yield, 94% purity) as yellow oil.

¹H NMR (400 MHz, CDCl₃), 4.76-4.88 (m, 2H), 4.20-4.35 (m, 1H), 4.06-4.17 (m, 2H), 3.01-3.29 (m, 3H), 2.56-2.70 (m, 2H), 2.44-2.55 (m, 1H), 2.27-2.40 (m, 1H), 1.85-1.95 (m, 1H), 1.55-1.73 (m, 3H), 1.18-1.52 (m, 64H), 1.13-1.17 (m, 12H), 0.88 (t, J=6.8 Hz, 12H).

### Step 4:

To a solution of 3-(dimethylamino) propanoic acid (0.4 g, 2.60 mmol, 1 eq, HCl), oxalyl dichloride (1.65 g, 13.02 mmol, 1.14 mL, 5 eq) in DCM (5 mL) was added DMF (19.03 mg, 260.41 μmol, 20.03 μL, 0.1 eq) at 0° C. The mixture was stirred at 20° C. for 2 hr. The reaction mixture was concentrated under reduced pressure to get compound 3-(dimethylamino)propanoyl chloride (0.35 g, crude, HCl) as yellow solid. To a solution of [8-(1-hexylnonoxy)-7,7-dimethyl-8-oxo-octyl] (2S,4S)-1-[8-(1-hexylnonoxy)-7,7-dimethyl-8-oxo-octyl]-4-hydroxy-pyrrolidine-2-carboxylate (0.4 g, 448.22 μmol, 1 eq), TEA (408.19 mg, 4.03 mmol, 561.48 μL, 9 eq). DMAP (5.48 mg, 44.82 μmol, 0.1 eq) in DCM (5 mL) was added the crude 3-(dimethylamino)propanoyl chloride (308.47 mg, 1.79 mmol, 4 eq, HCl) at 0° C. The mixture was stirred at 20° C. for 8 hr. The reaction mixture was diluted with H₂O 20 mL and extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=20/1 to 3/1, 3% NH₃·H₂O) and prep-HPLC (column: Phenomenex Gemini-NX 80*40 mm*3 μm; mobile phase: [H₂O (0.04% HCl)-ACN:THF=1:1]; gradient: 45%-95% B over 8.0 min) to give a compound [8-(1-hexylnonoxy)-7,7-dimethyl-8-oxo-octyl] (2S,4S)-4-[3-(dimethylamino)propanoyloxy]-1-[8-(1-hexylnonoxy)-7,7-dimethyl-8-oxo-octyl]pyrrolidine-2-carboxylate (0.066 g, 60.99 μmol, 86.39% yield, 95% purity, HCl) as yellow oil.

¹H NMR (400 MHz, CDCl₃), 11.52-13.38 (m, 2H), 5.20-5.49 (m, 1H), 4.67-4.83 (m, 2H), 4.01-4.48 (m, 4H), 3.44-3.55 (m, 2H), 3.18-3.37 (m, 2H), 2.95-3.13 (m, 2H), 2.71-2.87 (m, 6H), 2.48-2.65 (m, 1H), 2.08-2.24 (m, 2H), 1.59-1.78 (m, 4H), 1.05-1.44 (m, 76H), 0.81 (t, J=6.4 Hz, 12H).

LCMS: (M+H⁺): 991.8 @ 13.757 min.

### 9.14. Synthesis of 2443

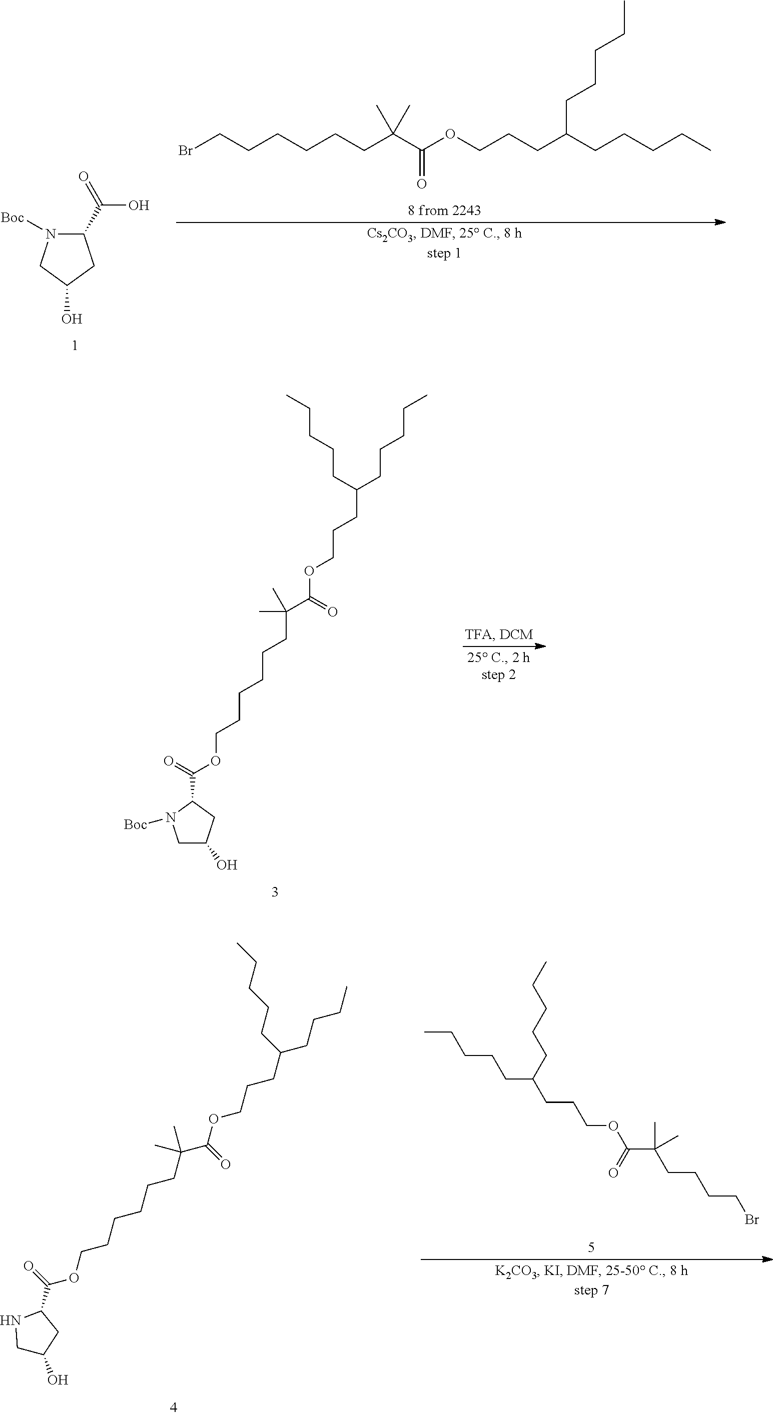

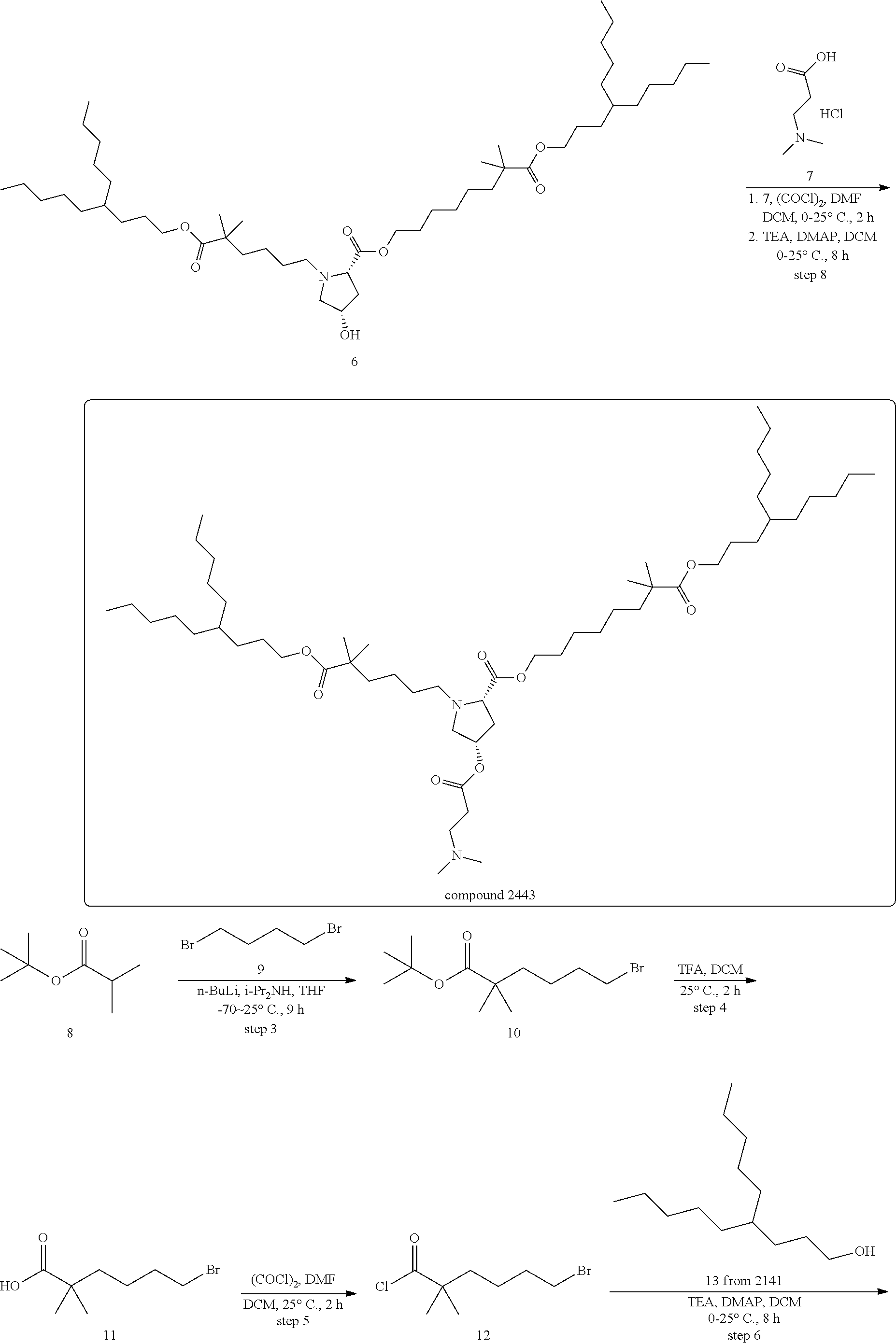

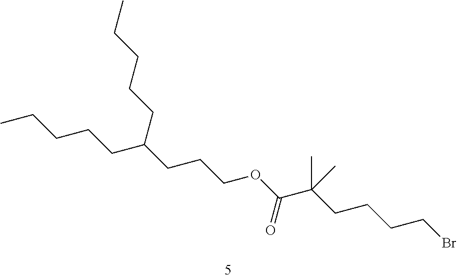 [SMILES: CCCCCC(CCCCC)CCCOC(=O)C(C)(C)CCCCBr]

### Step 1:

To a solution of (2S,4S)-1-tert-butoxycarbonyl-4-hydroxy-pyrrolidine-2-carboxylic acid (2 g, 8.65 mmol, 1 eq) and Cs₂CO₃ (6.20 g, 19.03 mmol, 2.2 eq) in DMF (25 mL) was added 4-pentylnonyl 8-bromo-2,2-dimethyl-octanoate (4.64 g, 10.38 mmol, 1.2 eq) at 25° C., under N₂ atmosphere. The mixture was stirred at 25° C. for 8 hr under N₂ atmosphere. The reaction mixture was added in H₂O 100 mL and extracted with EtOAc 150 mL (50 mL*3). The combined organic layers were washed with brine (150 mL), dried with anhydrous Na₂SO₄, filtered and concentrated under reduced pressure to give a residue. The residue was purified by column chromatography (SiO₂, Petroleum ether/Ethyl acetate=1/0 to 1/1) to give a compound O1-tert-butyl O2-[7,7-dimethyl-8-oxo-8-(4-pentylnonoxy)octyl] (2S,4S)-4-hydroxypyrrolidine-1,2-dicarboxylate (2.7 g, 3.36 mmol, 38.90% yield, 74.5% purity) as colorless oil.

¹H NMR (400 MHz, CDCl₃), 4.33-4.37 (m, 1H), 4.10-4.25 (m, 2H), 4.16 (t, J=6.4 Hz, 2H), 2.25-3.75 (m, 3H), 2.25-2.40 (m, 1H), 2.05-2.09 (m, 1H), 1.16-1.75 (m, 2H), 1.55-1.65 (m, 4H), 1.40-1.50 (m, 10H), 1.20-1.35 (m, 25H), 1.16 (s, 6H), 0.89 (t, J=6.8 Hz, 6H).

### Step 2:

To a solution of O1-tert-butyl O2-[7,7-dimethyl-8-oxo-8-(4-pentylnonoxy)octyl] (2S,4S)-4-hydroxypyrrolidine-1,2-dicarboxylate (1 g, 1.67 mmol, 1 eq) in DCM (10 mL) was added TFA (7.68 g, 67.31 mmol, 5 mL, 40.24 eq) and purged with N₂ for 3 times, and then the mixture was stirred at 25° C. for 2 hr under N₂ atmosphere. The reaction mixture was concentrated under reduced pressure to get a residue. Then the residue was dissolved with EtOAc (30 mL) and washed with water 60 mL (20 mL*3) and brine 40 mL (20 mL*2), dried with anhydrous Na₂SO₄, filtered and concentrated under reduced pressure to give a residue. The residue was purified by column chromatography (SiO₂, Petroleum ether/Ethyl acetate=20/1 to 1/0, 3% NH₃·H₂O) to give a compound [7,7-dimethyl-8-oxo-8-(4-pentylnonoxy)octyl] (2S,4S)-4-hydroxypyrrolidine-2-carboxylate (668 mg, 1.34 mmol, 80.24% yield) as colorless oil.

### Step 3:

To a solution of n-BuLi (2.5 M, 104.02 mL, 1.5 eq) in THF (250 mL) was added dropwise diisopropylamine (26.31 g, 260.04 mmol, 36.75 mL, 1.5 eq) at −40° C., under N₂, stirred for 0.5 h and then cooled to −70° C., the solution was added dropwise into a solution of tert-butyl 2-methylpropanoate (25 g, 173.36 mmol, 1 eq) in THF (200 mL), stirred at −70° C. for 0.5 h under N₂, a solution of 1,4-dibromobutane (67.38 g, 312.05 mmol, 37.64 mL, 1.8 eq) in THF (200 mL) was added dropwise into the mixture at −70° C., the mixture was stirred at 25° C. for 8 h under N₂. The mixture was cooled to 0° C. and then added slowly into aq. NH₄Cl solution (200 mL) under N₂ at 0° C. The mixture was stirred at 0° C. for 30 min, then the mixture was extracted with EtOAc (200 mL*3). The combined organic phases were washed with brine (100 mL*2), dried over Na₂SO₄ and filtered and the filtrate was concentrated under reduced pressure. The residue was purified by column chromatography (SiO₂, Petroleum ether/Ethyl acetate=1/0 to 50/1) to give compound Tert-butyl 6-bromo-2,2-dimethyl-hexanoate (45 g, 161.17 mmol, 92.97% yield) as colorless oil.

### Step 4:

A solution of tert-butyl 6-bromo-2,2-dimethyl-hexanoate (10 g, 35.81 mmol, 1 eq) in DCM (30 mL) and TFA (50.84 g, 445.89 mmol, 33.01 mL, 12.45 eq) was stirred at 25° C. for 2 h. The mixture was concentrated under reduced pressure. And then the dissolved with EtOAc (200 mL), washed with NaHCO₃ (200 mL*3), dried over Na₂SO₄, filtered and the filtrate was concentrated to give compound 6-bromo-2,2-dimethyl-hexanoic acid (30 g, crude) as colorless oil.

### Step 5:

To a solution of 6-bromo-2,2-dimethyl-hexanoic acid (4 g, 17.93 mmol, 1 eq) in DCM (150 mL) was added DMF (131.04 mg, 1.79 mmol, 137.94 μL, 0.1 eq) and (COCl)₂ (4.55 g, 35.86 mmol, 3.14 mL, 2 eq). The mixture was stirred at 25° C. for 2 hr. The reaction mixture was concentrated under reduced pressure to give compound 6-bromo-2,2-dimethyl-hexanoyl chloride (17 g, crude, 4 batches) as a yellow solid.

### Step 6:

To a solution of 4-pentylnonan-1-ol (1.78 g, 8.28 mmol, 1 eq), TEA (4.19 g, 41.40 mmol, 5.76 mL, 5 eq) and DMAP (202.30 mg, 1.66 mmol, 0.2 eq) in DCM (30 mL) was added dropwise 6-bromo-2,2-dimethyl-hexanoyl chloride (2 g, 8.28 mmol, 1 eq) in DCM (5 mL) at 0° C. After addition, the mixture was stirred at 25° C. for 8 h. The reaction mixture was concentrated under reduced pressure to get a residue. Then the residue was diluted with EtOAc 50 mL and washed with sat. NaHCO₃ aq. 300 mL (100 mL*3) and sat. NaCl aq. 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=20/1 to 0/1, 3% NH; H₂O) to give a compound 4-pentylnonyl 6-bromo-2,2-dimethyl-hexanoate (2.01 g, 4.79 mmol, 57.87% yield) as colorless oil.

### Step 7:

To a solution of [7,7-dimethyl-8-oxo-8-(4-pentylnonoxy)octyl] (2S,4S)-4-hydroxypyrrolidine-2-carboxylate (660 mg, 1.33 mmol, 1 eq), KI (44.02 mg, 265.19 μmol, 0.2 eq) and K₂CO₃ (366.51 mg, 2.65 mmol, 2 eq) in DMF (10 mL) was added 4-pentylnonyl 6-bromo-2,2-dimethyl-hexanoate (667.46 mg, 1.59 mmol, 1.2 eq) at 25° C., under N₂ atmosphere. And then the mixture was stirred at 50° C. for 8 hr under N₂ atmosphere. The reaction mixture was added in H₂O 100 mL and extracted with EtOAc 150 mL (50 mL*3). The combined organic layers were washed with sat. NaCl solution (150 mL), dried over Na₂SO₄, filtered and concentrated under reduced pressure to give a residue. The residue was purified by column chromatography (SiO₂, Petroleum ether/Ethyl acetate=1/0 to 1/1) to give a compound [7,7-dimethyl-8-oxo-8-(4-pentylnonoxy)octyl] (2S,4S)-1-[5,5-dimethyl-6-oxo-6-(4-pentylnonoxy) hexyl]-4-hydroxy-pyrrolidine-2-carboxylate (626 mg, 748.52 μmol, 56.45% yield) as colorless oil.

¹H NMR (400 MHz, CDCl₃), 4.12 (t, J=6.8 Hz, 2H), 4.02-4.06 (m, 4H), 3.10-3.30 (m, 1H), 3.05 (d, J=9.6 Hz, 1H), 2.60-2.70 (m, 2H), 2.40-2.60 (m, 1H), 2.25-2.40 (m, 1H), 1.85-1.95 (m, 1H), 1.55-1.70 (m, 8H), 1.50-1.55 (m, 4H), 1.40-1.50 (m, 2H), 1.20-1.40 (m, 47H), 1.16 (s, 12H), 0.89 (t, J=6.8 Hz, 12H).

### Step 8:

A mixture of 3-(dimethylamino) propanoic acid (300 mg, 1.95 mmol, 1 eq, HCl) in DCM (20 mL) was added (COCl)₂ (1.24 g, 9.77 mmol, 854.82 μL, 5 eq) and DMF (7.14 mg, 97.65 μmol, 7.51 μL, 0.05 eq) dropwise at 0)° C., under N₂ atmosphere. The mixture was degassed and purged with N₂ for 3 times, and then stirred at 25° C. for 2 hr under N₂ atmosphere. The reaction mixture was concentrated under reduced pressure to give a compound 3-(dimethylamino)propanoyl chloride (310 mg, 1.80 mmol, 92.25% yield, HCl) as yellow solid. To a solution of [7,7-dimethyl-8-oxo-8-(4-pentylnonoxy)octyl] (2S,4S)-1-[5,5-dimethyl-6-oxo-6-(4-pentylnonoxy) hexyl]-4-hydroxy-pyrrolidine-2-carboxylate (0.3 g, 358.72 μmol, 1 eq), TEA (254.09 mg, 2.51 mmol, 349.50 μL, 7 eq) and DMAP (21.91 mg, 179.36 μmol, 0.5 eq) in DCM (20 mL) was added 3-(dimethylamino)propanoyl chloride (308.59 mg, 1.79 mmol, 5 eq, HCl) in DCM (5 mL) dropwise at 0° C. After addition, the mixture was stirred at 25° C. for 8 h. The reaction mixture was added in H₂O 100 mL and extracted with EtOAc 150 mL (50 mL*3). The combined organic layers were washed with brine (150 mL) and water 60 mL (20 mL*3), dried with anhydrous Na₂SO₄, filtered and concentrated under reduced pressure to give a residue. The residue was purified by column chromatography (SiO₂, Petroleum ether/Ethyl acetate=10/1 to 0/1) to give a compound [7,7-dimethyl-8-oxo-8-(4-pentylnonoxy)octyl] (2S,4S)-4-[3-(dimethylamino)propanoyloxy]-1-[5,5-dimethyl-6-oxo-6-(4-pentylnonoxy) hexyl]pyrrolidine-2-carboxylate (18 mg, 96.21 μmol, 26.82% yield) as colorless oil.

¹H NMR (400 MHz, CDCl₃), 5.19-5.23 (m, 1H), 4.12-4.15 (m, 2H), 4.01-4.06 (m, 4H), 3.23-3.26 (d, J=11.2 Hz, 1H), 3.11 (t, J=8.4 Hz, 1H), 2.75-2.79 (m, 1H), 2.60-2.63 (m, 4H), 2.47-2.49 (m, 2H), 2.24-2.38 (m, 7H), 2.03-2.12 (m, 1H), 1.59-1.62 (m, 6H), 1.44-1.50 (m, 6H), 1.25-1.30 (m, 46H), 1.16-1.17 (m, 12H), 0.89 (t, J=6.8 Hz, 12H).

LCMS: (M+H⁺): 936.3 @ 10.398 min.

### 9.15. Synthesis of 2454

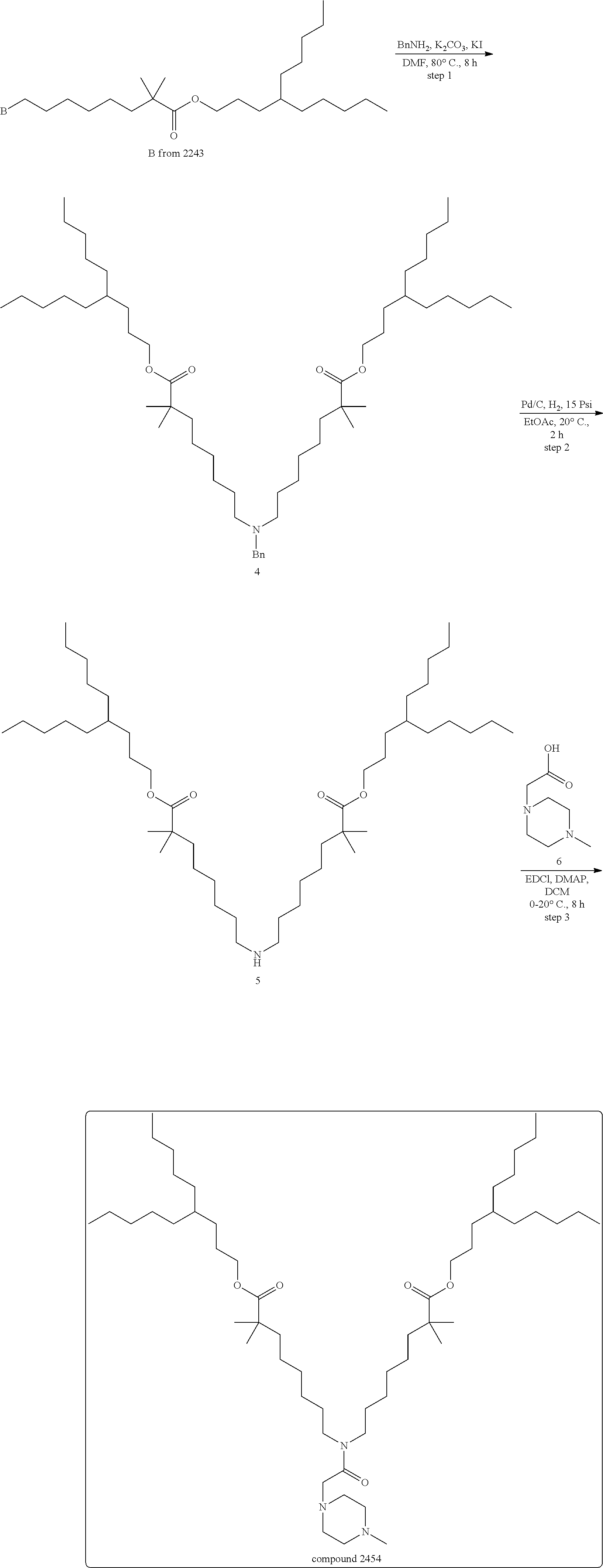

### Step 1:

To a solution of 4-pentylnonyl 8-bromo-2,2-dimethyl-octanoate (8 g, 17.88 mmol, 2 eq), phenylmethanamine (957.72 mg, 8.94 mmol, 974.29 μL, 1 eq) in DMF (80 mL) was added K₂CO₃ (6.18 g, 44.69 mmol, 5 eq), KI (1.48 g, 8.94 mmol, 1 eq) and stirred at 80° C. for 8 hr. The reaction mixture was diluted with H₂O 20 mL and extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=10/1 to 1/1) to give compound 4-pentylnonyl 8-[benzyl-[7,7-dimethyl-8-oxo-8-(4-pentylnonoxy)octyl]amino]-2,2-dimethyl-octanoate (4.5 g, crude) as colorless oil.

### Step 2:

A solution of 4-pentylnonyl 8-[benzyl-[7,7-dimethyl-8-oxo-8-(4-pentylnonoxy)octyl]amino]-2,2-dimethyl-octanoate (2 g, 2.38 mmol, 1 eq) in EtOAc (20 mL) was added to Pd/C (1.00 g, 939.67 μmol, 10% purity, 3.95e-1 eq) under N₂. The suspension was degassed under vacuum and purged with H₂ for 3 times. The mixture was stirred under H₂ under 15 psi at 20° C. for 2 hours. The mixture is filtered through celite and the solvent is removed under reduced pressure to give a residue. The residue was purified by column chromatography (SiO₂, Petroleum ether/Ethyl acetate=1/0 to 1/1) to give compound 4-pentylnonyl 8-[[7,7-dimethyl-8-oxo-8-(4-pentylnonoxy)octyl]amino]-2,2-dimethyl-octanoate (0.9 g, 1.20 mmol, 50.41% yield) as colorless oil.

### Step 3:

To a solution of 2-(4-methylpiperazin-1-yl) acetic acid (227.72 mg, 1.44 mmol, 1.2 eq), EDCI (344.94 mg, 1.80 mmol, 1.5 eq), DMAP (73.27 mg, 599.79 μmol, 0.5 eq) in DCM (10 mL) was added 4-pentylnonyl 8-[[7,7-dimethyl-8-oxo-8-(4-pentylnonoxy)octyl]amino]-2,2-dimethyl-octanoate (0.9 g, 1.20 mmol, 1 eq) at 0° C. The mixture was stirred at 20° C. for 8 hr. The reaction mixture was diluted with H₂O 20 mL and extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=1/0 to 1/0). The combined organic phase was diluted with hexane 20 mL, washed with ACN 60 mL (20 mL*3), and the hexane phase was concentrated under reduced pressure to give compound 4-pentylnonyl 8-[[7,7-dimethyl-8-oxo-8-(4-pentylnonoxy)octyl]-[2-(4-methylpiperazin-1-yl) acetyl]amino]-2,2-dimethyl-octanoate (185 mg, 196.54 μmol, 17.32% yield, 94.6% purity) as colorless oil.

¹H NMR (400 MHz, CDCl₃), 4.01-4.05 (m, 4H), 3.25-3.29 (m, 4H), 3.14 (s, 2H), 2.29-2.55 (m, 11H), 1.50-1.59 (m, 12H), 1.15-1.29 (m, 62H), 0.89 (t, J=6.8 Hz, 12H).

LCMS: (M+H⁺): 890.7 @ 14.693 min.

### 9.16. Synthesis of 2481

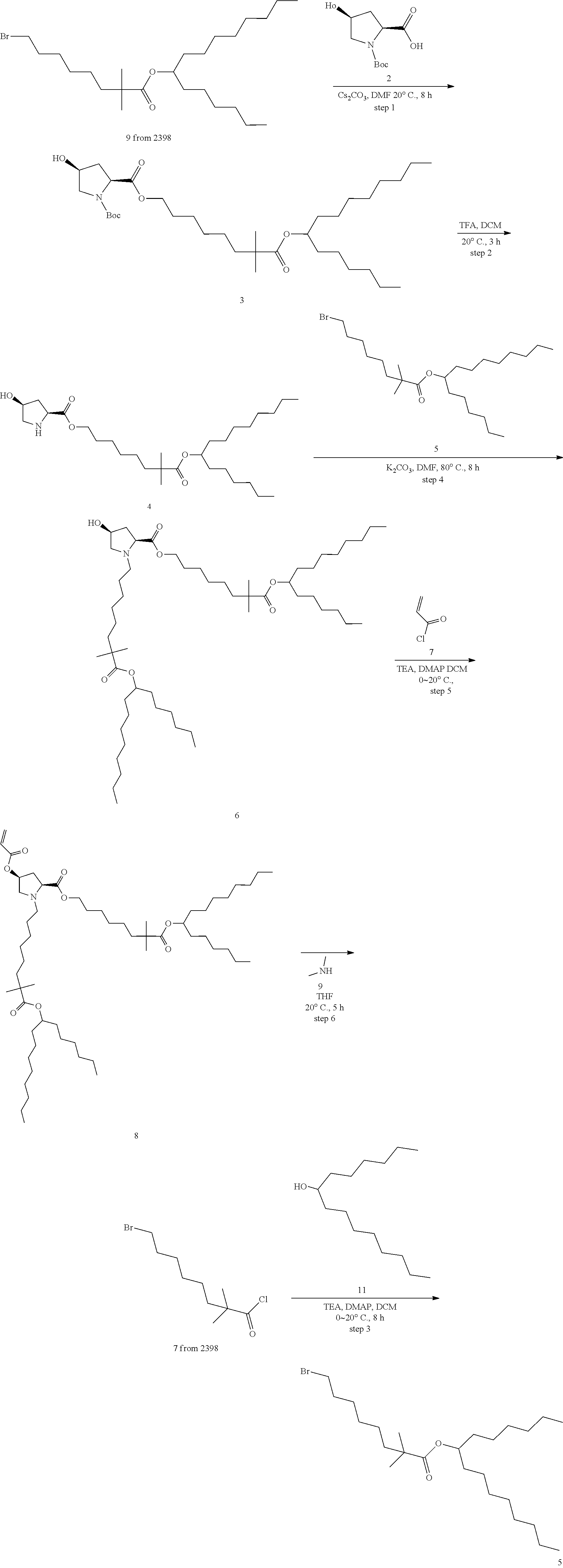

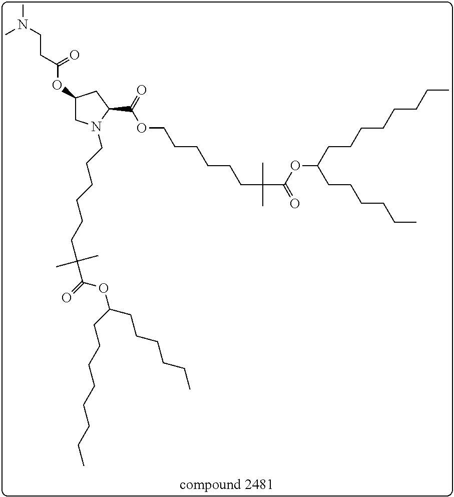 [SMILES: CCCCCCCCC(CCCCCC)OC(=O)C(C)(C)CCCCCCOC(=O)[C@@H]1C[C@H](OC(=O)CCN(C)C)CN1CCCCCCC(C)(C)C(=O)OC(CCCCCC)CCCCCCCC]

### Step 1:

A mixture of (2S,4S)-1-tert-butoxycarbonyl-4-hydroxy-pyrrolidine-2-carboxylic acid (4.29 g, 18.57 mmol, 1 eq), 1-octylnonyl 8-bromo-2,2-dimethyl-octanoate (10 g, 20.42 mmol, 1.1 eq), Cs₂CO₃ (13.31 g, 40.85 mmol, 2.2 eq) in DMF (200 mL) was stirred at 20° C. for 8 hr under N₂ atmosphere. The combined organic phase was diluted with EtOAc 80 mL and washed with water 150 mL (50 mL*3) and brine 200 mL (100 mL*2), dried with anhydrous Na₂SO₄, filtered and concentrated under reduced pressure to give a residue. The residue was purified by column chromatography (SiO₂, Petroleum ether/Ethyl acetate=10/1 to 3/1, added 0.5% NH₃·H₂O) to give compound O1-tert-butyl O2-[7,7-dimethyl-8-(1-octylnonoxy)-8-oxo-octyl] (2S,4S)-4-hydroxypyrrolidine-1,2-dicarboxylate (3 g, 4.69 mmol, 25.25% yield) as colorless oil.

### Step 2:

To a solution of O1-tert-butyl O2-[7,7-dimethyl-8-(1-octylnonoxy)-8-oxo-octyl] (2S,4S)-4-hydroxypyrrolidine-1,2-dicarboxylate (3 g, 4.69 mmol, 1 eq) in DCM (12 mL) was added TFA (6.14 g, 53.85 mmol, 4 mL, 11.49 eq). The mixture was stirred at 20° C. for 3 hr. The reaction mixture was adjusted pH to 7 with saturated NaHCO₃ aqueous and extracted with EtOAc 60 mL (20 mL*3), dried over Na₂SO₄, filtered and concentrated under reduced pressure to give a residue. The residue was purified by column chromatography (SiO₂, Petroleum ether/Ethyl acetate=5/1 to 0.1, added 3% NH₃·H₂O) to give compound [7,7-dimethyl-8-(1-octylnonoxy)-8-oxo-octyl] (2S,4S)-4-hydroxypyrrolidine-2-carboxylate (2 g, 3.70 mmol, 79.03% yield) as yellow oil.

¹H NMR (400 MHz, CDCl₃), 4.82-4.85 (m, 1H), 4.35-4.40 (m, 1H), 4.11-4.17 (m, 2H), 3.79-3.82 (m, 1H), 3.11-3.14 (m, 1H), 2.97-3.01 (m, 1H), 2.27-2.29 (m, 1H), 1.96-2.06 (m, 1H), 1.63-1.65 (m, 2H), 1.49-1.52 (m, 4H), 1.26-1.33 (m, 32H), 1.15 (s, 6H), 0.88 (t, J=6.8 Hz, 6H).

### Step 3:

To a solution of 8-bromo-2,2-dimethyl-octanoyl chloride (10 g, 37.09 mmol, 1 eq), TEA (18.77 g, 185.46 mmol, 25.81 mL, 5 eq). DMAP (906.29 mg, 7.42 mmol, 0.2 eq) in DCM (100 mL) was added pentadecan-7-ol (8.47 g, 37.09 mmol, 1 eq) at 0° C. The mixture was stirred at 20° C. for 8 hr. The reaction mixture was diluted with H₂O 200 mL and extracted with EtOAc 600 mL (200 mL*3). The combined organic layers were dried over Na₂SO₄, filtered and concentrated under reduced pressure to give a residue. The residue was purified by column chromatography (SiO₂, Petroleum ether/Ethyl acetate=1/0 to 1/0 to give compound 1-hexylnonyl 8-bromo-2,2-dimethyl-octanoate (17 g, crude) as yellow oil.

### Step 4:

To a solution of [7,7-dimethyl-8-(1-octylnonoxy)-8-oxo-octyl] (2S,4S)-4-hydroxypyrrolidine-2-carboxylate (2 g, 3.70 mmol, 1 eq), 1-hexylnonyl 8-bromo-2,2-dimethyl-octanoate (2.05 g, 4.45 mmol, 1.2 eq) in DMF (20 mL) was added K₂CO₃ (1.54 g, 11.11 mmol, 3 eq). The mixture was stirred at 80° C. for 8 hr. The reaction mixture was diluted with H₂O 50 mL and extracted with EtOAc 120 mL (40 mL*3). The combined organic layers were dried over Na₂SO₄, filtered and concentrated under reduced pressure to give a residue. The residue was purified by column chromatography (SiO₂, Petroleum ether/Ethyl acetate=1/0 to 10/1) to give compound [7,7-dimethyl-8-(1-octylnonoxy)-8-oxo-octyl] (2S,4S)-1-[8-(1-hexylnonoxy)-7,7-dimethyl-8-oxo-octyl]-4-hydroxy-pyrrolidine-2-carboxylate (2.3 g, 2.50 mmol, 76.67% yield, 100% purity) as yellow oil.

¹H NMR (400 MHz, CDCl₃), 4.80-4.86 (m, 2H), 4.25-4.27 (m, 1H), 4.11 (t, J=6.8 Hz, 2H), 3.04-3.25 (m, 2H), 2.60-2.63 (m, 2H), 2.30-2.52 (m, 2H), 1.90-1.95 (m, 1H), 1.60-1.64 (m, 2H), 1.50-1.53 (m, 14H), 1.26-1.31 (m, 56H), 1.15 (d, J=2.0 Hz, 12H), 0.88 (t, J=6.4 Hz, 12H).

### Step 5:

To a solution of [7,7-dimethyl-8-(1-octylnonoxy)-8-oxo-octyl] (2S,4S)-1-[8-(1-hexylnonoxy)-7,7-dimethyl-8-oxo-octyl]-4-hydroxy-pyrrolidine-2-carboxylate (2.3 g, 2.50) mmol, 1 eq), prop-2-enoyl chloride (452.31 mg, 5.00 mmol, 407.48 μL, 2 eq). DMAP (30.53 mg, 249.87 μmol, 0.1 eq) in DCM (20 mL) was added TEA (2.28 g, 22.49 mmol, 3.13 ml . . . 9) eq) at 0° C. The mixture was stirred at 20° C. for 8 hr. The reaction mixture was diluted with H₂O 100 mL and extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=1/0 to 3/1, added 0.5% NH₃·H₂O) to give compound [7,7-dimethyl-8-(1-octylnonoxy)-8-oxo-octyl] (2S,4S)-1-[8-(1-hexylnonoxy)-7,7-dimethyl-8-oxo-octyl]-4-prop-2-enoyloxy-pyrrolidine-2-carboxylate (880 mg, 903.01 μmol, 88.00% yield) as yellow oil.

### Step 6:

A mixture of [7,7-dimethyl-8-(1-octylnonoxy)-8-oxo-octyl] (2S,4S)-1-[8-(1-hexylnonoxy)-7,7-dimethyl-8-oxo-octyl]-4-prop-2-enoyloxy-pyrrolidine-2-carboxylate (0.4 g, 410.46 μmol, 1 eq) in N-methylmethanamine (2 M, 49.35 mL, 240.48 eq) was degassed and purged with N₂ for 3 times, and then the mixture was stirred at 20° C. for 5 hr under N₂ atmosphere. The reaction mixture was concentrated under reduced pressure to get a residue. The residue was purified by column chromatography (SiO₂, Petroleum ether/Ethyl acetate=10/1 to 3/1, added 3% NH₃·H₂O) to give compound [7,7-dimethyl-8-(1-octylnonoxy)-8-oxo-octyl] (2S,4S)-4-[3-(dimethylamino)propanoyl oxy]-1-[8-(1-hexylnonoxy)-7,7-dimethyl-8-oxo-octyl]pyrrolidine-2-carboxylate (148 mg, 145.15 μmol, 35.36% yield) as yellow oil.

¹H NMR (400 MHz, CDCl₃), 5.18-5.26 (m, 1H), 4.82-4.85 (m, 2H), 4.10-4.13 (m, 2H), 3.07-3.26 (m, 2H), 2.70-2.78 (m, 1H), 2.47-2.63 (m, 6H), 2.23-2.27 (m, 7H), 2.03-2.06 (m, 1H), 1.60-1.65 (m, 2H), 1.50-1.57 (m, 12H), 1.26-1.43 (m, 58H), 1.15 (d, J=3.2 Hz, 12H), 0.86-0.90 (m, 12H). LCMS: (½M+H⁺): 510.4 @ 10.581 min.

### 9.17. Synthesis of 2483

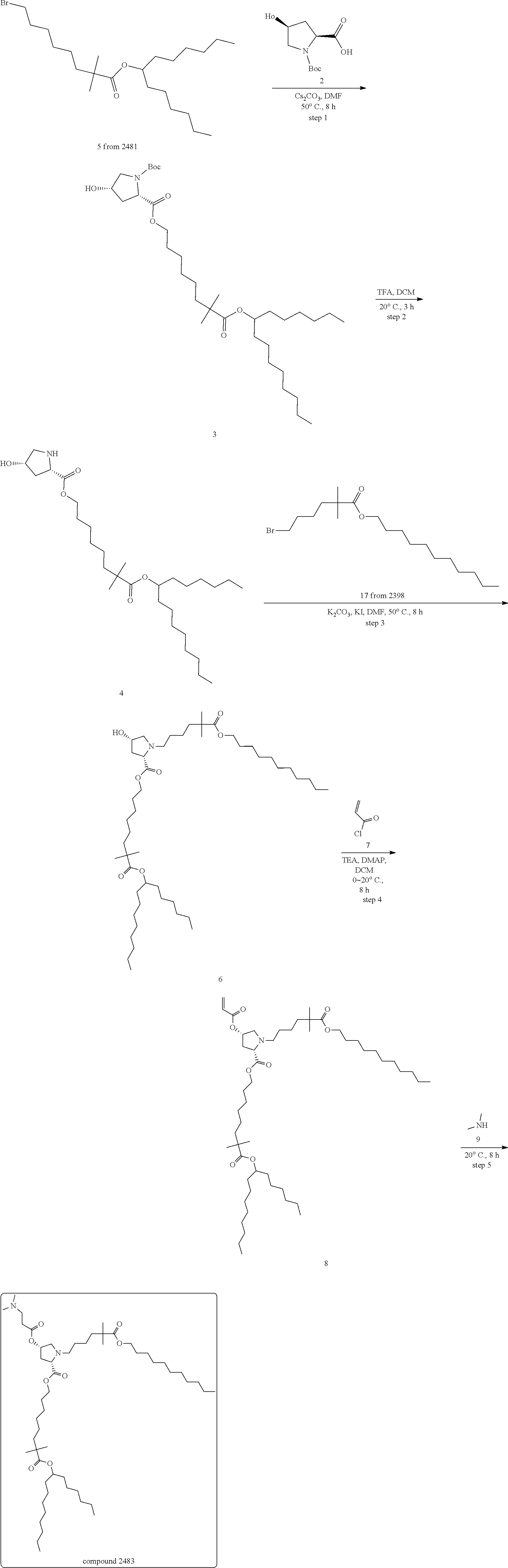

### Step 1:

To a solution of (2S,4S)-1-tert-butoxycarbonyl-4-hydroxy-pyrrolidine-2-carboxylic acid (1.46 g, 6.32 mmol, 1 eq) and 1-hexylnonyl 8-bromo-2,2-dimethyl-octanoate (3.5 g, 7.58 mmol, 1.2 eq) in DMF (40 mL) was added Cs₂CO₃ (4.53 g, 13.90 mmol, 2.2 eq). Then the mixture was stirred at 50° C. for 8 hr. The reaction mixture diluted with by addition H₂O 50 mL, and then extracted with EtOAc 45 mL (15 mL*3). The combined organic layers were washed with sat, brine 40 mL (20 mL*2), dried over Na₂SO₄, filtered and concentrated under reduced pressure to give a residue. The residue was purified by column chromatography (SiO₂, Petroleum ether/Ethyl acetate=1/0 to 3/1) to give compound O1-tert-butyl O2-[8-(1-hexylnonoxy)-7,7-dimethyl-8-oxo-octyl] (2S,4S)-4-hydroxypyrrolidine-1,2-dicarboxylate (3.5 g, 5.72 mmol, 90.52% yield, 100% purity) as colorless oil.

### Step 2:

To a solution of O1-tert-butyl O2-[8-(1-hexylnonoxy)-7,7-dimethyl-8-oxo-octyl] (2S,4S)-4-hydroxypyrrolidine-1,2-dicarboxylate (3.5 g, 5.72 mmol, 1 eq) in DCM (20 mL) was added TFA (15.35 g, 134.62 mmol, 10 mL, 23.54 eq). Then the mixture was stirred at 20° C., for 3 hr. The reaction mixture was adjusted pH to 8 with sat. NaHCO₃, and then extracted EtOAc 90 mL (30 mL*3). The combined organic layers were dried over Na₂SO₄, filtered and concentrated under reduced pressure to give compound [8-(1-hexylnonoxy)-7,7-dimethyl-8-oxo-octyl] (2S,4S)-4-hydroxypyrrolidine-2-carboxylate (3 g, crude) as yellow oil.

¹H NMR (400 MHz, CDCl₃), 4.81-4.84 (m, 1H), 4.57-4.61 (m, 1H), 4.38-4.41 (m, 1H), 4.25-4.32 (m, 1H), 4.12-4.17 (m, 1H), 3.57 (d, J=12.0 Hz, 1H), 3.29-3.33 (m, 1H), 2.41-2.50 (m, 2H), 1.64-1.67 (m, 2H), 1.48-1.54 (m, 6H), 1.26-1.45 (m, 26H), 1.15 (s, 6H), 0.88 (t, J=6.4 Hz, 6H).

### Step 3:

To a solution of [8-(1-hexylnonoxy)-7,7-dimethyl-8-oxo-octyl] (2S,4S)-4-hydroxypyrrolidine-2-carboxylate (3 g, 5.86 mmol, 1 eq) and undecyl 6-bromo-2,2-dimethyl-hexanoate (2.65 g, 7.03 mmol, 1.2 eq) in DMF (50 mL) was added K₂CO₃ (2.43 g, 17.59 mmol, 3 eq) and KI (1.95 g, 11.72 mmol, 2 eq) in sequence. Then the mixture was stirred at 50° C. for 8 hr. The reaction mixture diluted with by addition H₂O 50 mL, and then extracted with EtOAc 45 mL (15 mL*3). The combined organic layers were washed with sat, brine 40 mL (20 mL*2), dried over Na₂SO₄, filtered and concentrated under reduced pressure to give a residue. The residue was purified by column chromatography (SiO₂, Petroleum ether/Ethyl acetate=1/0 to 5/1) to give compound [8-(1-hexylnonoxy)-7,7-dimethyl-8-oxo-octyl] (2S,4S)-1-(5,5-dimethyl-6-oxo-6-undecoxy-hexyl)-4-hydroxy-pyrrolidine-2-carboxylate (0.9 g, 1.11 mmol, 19.00% yield) as colorless oil.

### Step 4:

To a solution of [8-(1-hexylnonoxy)-7,7-dimethyl-8-oxo-octyl] (2S,4S)-1-(5,5-dimethyl-6-oxo-6-undecoxy-hexyl)-4-hydroxy-pyrrolidine-2-carboxylate (0.9 g, 1.11 mmol, 1 eq) in DCM (10 mL) was added TEA (1.13 g, 11.14 mmol, 1.55 mL, 10 eq) and DMAP (68.02 mg, 556.75 μmol, 0.5 eq) and prop-2-enoyl chloride (503.90 mg, 5.57 mmol, 452.34 μL, 5 eq) in DCM (3 mL) in sequence at 0° C. Then the mixture was stirred at 20° C. for 8 hr. The reaction mixture diluted with by addition H₂O 30 mL, and then extracted with EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate=1/0 to 10/1) to give compound [8-(1-hexylnonoxy)-7,7-dimethyl-8-oxo-octyl] (2S,4S)-1-(5,5-dimethyl-6-oxo-6-undecoxy-hexyl)-4-prop-2-enoyloxy-pyrrolidine-2-carboxylate (400 mg, 463.87 μmol, 41.66% yield) as colorless oil.

¹H NMR (400 MHz, CDCl₃), 6.39-6.44 (m, 1H), 6.10-6.17 (m, 1H), 5.80-5.83 (m, 1H), 5.25-5.26 (m, 1H), 4.82-4.85 (m, 1H), 4.10-4.13 (m, 2H), 4.03 (t, J=6.8 Hz, 2H), 3.29 (d, J=10.8 Hz, 1H), 3.13 (t, J=8.4 Hz, 1H), 2.72-2.80 (m, 1H), 2.60-2.65 (m, 2H), 2.25-2.35 (m, 1H), 2.06-2.12 (m, 1H), 1.60-1.68 (m, 4H), 1.45-1.55 (m, 8H), 1.26-1.30 (m, 46H), 1.15 (s, 12H), 0.86-0.89 (m, 9H).

### Step 5:

A solution of [8-(1-hexylnonoxy)-7,7-dimethyl-8-oxo-octyl] (2S,4S)-1-(5,5-dimethyl-6-oxo-6-undecoxy-hexyl)-4-prop-2-enoyloxy-pyrrolidine-2-carboxy late (400 mg, 463.87 μmol, 1 eq) in N-methylmethanamine (2 M/THF, 49.35 mL, 212.79 eq) was stirred at 20° C. for 8 hr. The reaction mixture was concentrated under reduced pressure to remove solvent. The residue was purified by column chromatography (SiO₂, Petroleum ether/Ethyl acetate=10/1 to 1/1) to give compound [8-(1-hexylnonoxy)-7,7-dimethyl-8-oxo-octyl] (2S,4S)-4-[3-(dimethylamino)propanoyloxy]-1-(5,5-dimethyl-6-oxo-6-undecoxy-hexyl)pyrrolidine-2-carboxylate (86 mg, 165.31 μmol, 20.43% yield) as colorless oil.

¹H NMR (400 MHz, CDCl₃), 5.16-5.24 (m, 1H), 4.80-4.86 (m, 1H), 4.02-4.13 (m, 4H), 3.24 (d, J=10.8 Hz, 1H), 3.10 (t, J=8.0 Hz, 1H), 2.74-2.76 (m, 1H), 2.46-2.61 (m, 6H), 2.23-2.28 (m, 7H), 2.03-2.05 (m, 1H), 1.59-1.65 (m, 4H), 1.49-1.52 (m, 10H), 1.26-1.43 (m, 44H), 1.15 (s, 12H), 0.89 (t, J=4.4 Hz, 9H). LCMS: (M+H⁺): 907.6 @ 11.570 min.

### 9.18. Synthesis of 2383

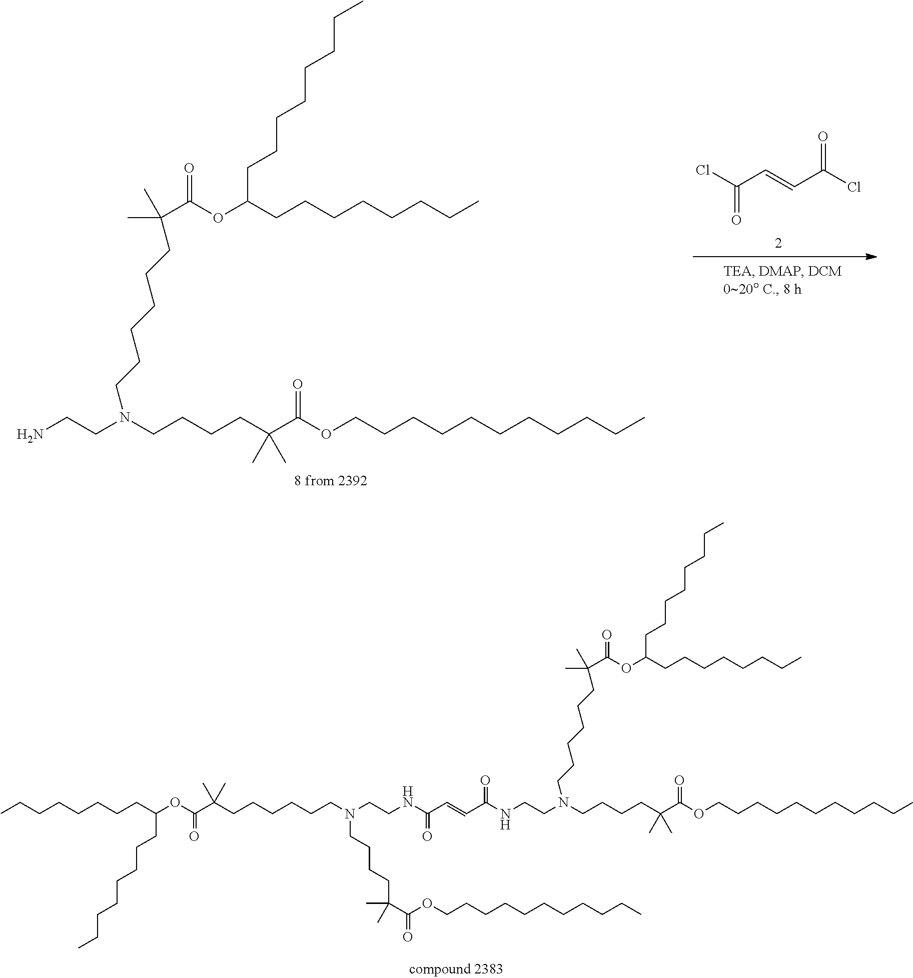

### Step 1:

To a solution of 1-octylnonyl 8-[2-aminoethyl-(5,5-dimethyl-6-oxo-6-undecoxy-hexyl)amino]-2,2-dimethyl-octanoate (1 g, 1.31 mmol, 2 eq) in DCM (10 mL) was added TEA (198.34 mg, 1.96 mmol, 272.81 μL, 3 eq). DMAP (39.91 mg, 326.68 μmol, 0.5 eq) and (E)-but-2-enedioyl dichloride (99.94 mg, 653.35 μmol, 70.88 μL, 1 eq) at 0° C. The mixture was stirred at 20° C. for 8 hours. The reaction mixture was quenched by addition H₂O 10 mL at 0° C., and then extracted with EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate=1/0 to 0/1). Then the residue was purified by prep-HPLC (column: X-Select CSH Phenyl-Hexyl 100*30 5 u; mobile phase: [H₂O (0.04% HCl)-THF:ACN=1:3]; gradient: 50%-90% B over 8.0 min). The mixture was adjusted pH to 8 with sat. NaHCO₃, extracted with EtOAc 60 mL (20 mL*3). The combined organic layers were over Na₂SO₄, filtered and concentrated under reduced pressure. The residue was purified by prep-TLC (SiO₂, EA:MeOH=10:1) to give compound 1-octylnonyl 8-[2-[[(E)-4-[2-[[7,7-dimethyl-8-(1-octylnonoxy)-8-oxo-octyl]-(5,5-dimethyl-6-oxo-6-undecoxy-hexyl)amino]ethylamino]-4-oxo-but-2-enoyl]amino]ethyl-(5,5-dimethyl-6-oxo-6-undecoxy-hexyl)amino]-2,2-dimethyl-octanoate (29 mg, 13.99 μmol, 56.84% yield, 98% purity) as colorless oil.

¹H NMR (400 MHz, CDCl₃), 6.87 (s, 2H), 6.52 (t, J=4.4 Hz, 2H), 4.82-4.85 (m, 2H), 4.05 (t, J=6.8 Hz, 4H), 3.35-3.39 (m, 4H), 2.55 (t, J=5.6 Hz, 4H), 2.37-2.41 (m, 8H), 1.58-1.62 (m, 4H), 1.49-1.54 (m, 16H), 1.36-1.42 (m, 10H), 1.20-1.36 (m, 94H), 1.16 (d, J=4.4 Hz, 24H), 0.86-0.90 (m, 18H) (½M+H⁺): 806.0. LCMS: (½M+H⁺): 806.0@ 12.035 min.

### 9.19. Synthesis of 2482

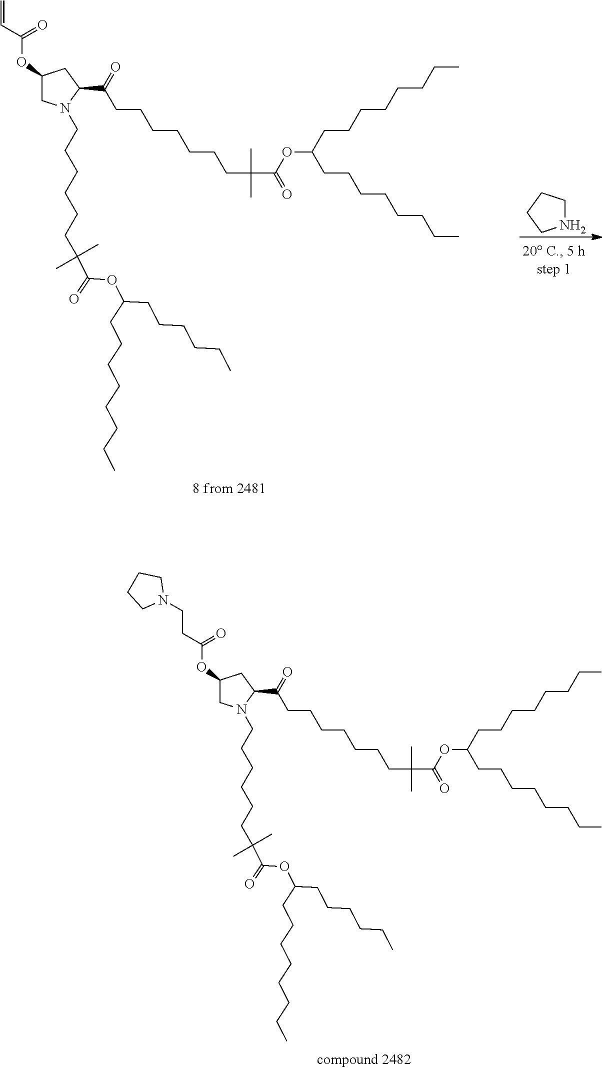 [SMILES: C=CC(=O)O[C@H]1C[C@@H](C(=O)CCCCCCCC(C)(C)C(=O)OC(CCCCCCCC)CCCCCCCC)N(CCCCCCC(C)(C)C(=O)OC(CCCCCC)CCCCCCCC)C1 ; C1CCNC1 ; CCCCCCCCC(CCCCCC)OC(=O)C(C)(C)CCCCCCN1C[C@@H](OC(=O)CCN2CCCC2)C[C@H]1C(=O)CCCCCCCC(C)(C)C(=O)OC(CCCCCCCC)CCCCCCCC]

### Step 1:

A mixture of [7,7-dimethyl-8-(1-octylnonoxy)-8-oxo-octyl] (2S,4S)-1-[8-(1-hexylnonoxy)-7,7-dimethyl-8-oxo-octyl]-4-prop-2-enoyloxy-pyrrolidine-2-carboxylate (0.4 g, 410.46 μmol, 1 eq) in pyrrolidine (145.96 mg, 2.05 mmol, 171.31 μL, 5 eq) was degassed and purged with N₂ for 3 times, and then the mixture was stirred at 20° C. for 5 hr under N₂ atmosphere. The reaction mixture was concentrated under reduced pressure to get a residue. The residue was purified by column chromatography (SiO₂, Petroleum ether/Ethyl acetate=10/1 to 3/1, added 3% NH₃·H₂O) to give compound [7,7-dimethyl-8-(1-octylnonoxy)-8-oxo-octyl] (2S,4S)-1-[8-(1-hexylnonoxy)-7,7-dimethyl-8-oxo-octyl]-4-(3-pyrrolidin-1-ylpropanoyloxy)pyrrolidine-2-carboxylate (0.185 g, 176.92 μmol, 43.10% yield) as yellow oil. ¹H NMR (400 MHz, CDCl₃), 5.19-5.22 (m, 1H), 4.80-4.86 (m, 2H), 4.09-4.13 (m, 2H), 3.08-3.26 (m, 2H), 2.73-2.77 (m, 3H), 2.50-2.60 (m, 8H), 2.25-2.27 (m, 1H), 2.00-2.07 (m, 1H), 1.72-1.82 (m, 4H), 1.61-1.65 (m, 2H), 1.50-1.58 (m, 12H), 1.18-1.36 (m, 58H), 1.15 (d, J=3.2 Hz, 12H), 0.86-0.90 (m, 12H). (M+H⁺): 1045.8.

LCMS: (M+H⁺): 1045.8 @ 10.833 min.

### 9.20. Synthesis of 2486

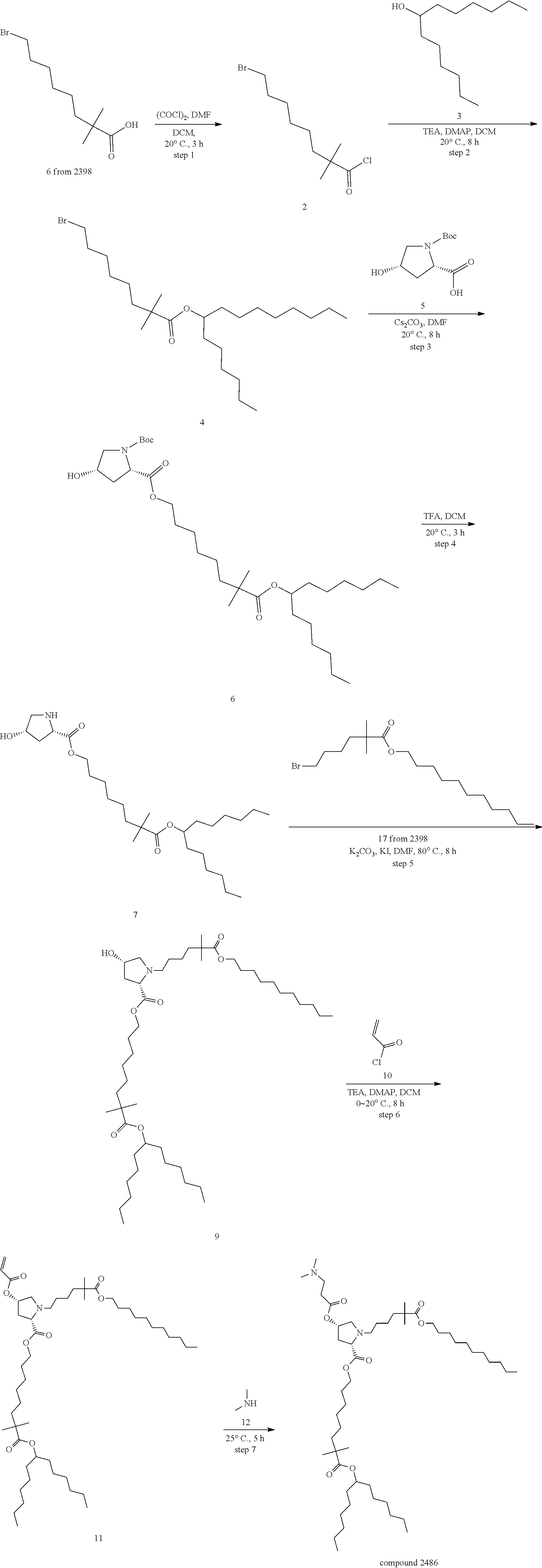

### Step 1:

A mixture of 8-bromo-2,2-dimethyl-octanoic acid (7 g, 27.87 mmol, 1 eq) in DCM (100 mL) was added (COCl)₂ (17.69 g, 139.35 mmol, 12.20 mL, 5 eq), DMF (40.74 mg, 557.41 μmol, 42.89 μL, 0.02 eq) at 20° C. The mixture was stirred at 20° C. for 3 hr under N₂ atmosphere. The reaction mixture was concentrated under reduced pressure to give compound 8-bromo-2,2-dimethyl-octanoyl chloride (7.5 g, crude) as yellow oil.

### Step 2:

To a solution of 8-bromo-2,2-dimethyl-octanoyl chloride (7.5 g, 27.82 mmol, 1.1 eq), TEA (12.80 g, 126.45 mmol, 17.60 mL, 5 eq), DMAP (617.92 mg, 5.06 mmol, 0.2 eq) in DCM (100 mL) was added tridecan-7-ol (5.07 g, 25.29 mmol, 1 eq) at 20° C. The mixture was stirred at 20° C. for 8 hr under N₂ atmosphere. The reaction mixture was diluted with H₂O 100 mL and extracted with EtOAc 300 mL (100 mL*3). The combined organic layers were dried over Na₂SO₄, filtered and concentrated under reduced pressure to give a residue. The residue was purified by column chromatography (SiO₂, Petroleum ether/Ethyl acetate=1/0 to 1/0) to give compound 1-hexylheptyl 8-bromo-2,2-dimethyl-octanoate (5.8 g, 13.38 mmol, 58.00% yield) as colorless oil.

¹H NMR (400 MHz, CDCl₃), 4.80-4.87 (m, 1H), 3.38-3.54 (m, 2H), 1.75-1.92 (m, 2H), 1.48-1.56 (m, 4H), 1.21-1.36 (m, 24H), 1.16 (s, 6H), 0.88 (t, J=6.4 Hz, 6H).

### Step 3:

A mixture of 1-hexylheptyl 8-bromo-2,2-dimethyl-octanoate (5.8 g, 13.38 mmol, 1.2 eq), (2S,4S)-1-tert-butoxy carbonyl-4-hydroxy-pyrrolidine-2-carboxylic acid (2.58 g, 11.15 mmol, 1 eq), Cs₂CO₃ (7.99 g, 24.53 mmol, 2.2 eq) in DMF (60 mL) was stirred at 20° C. for 8 hr under N₂ atmosphere. The combined organic phase was diluted with EtOAc 60 mL and washed with water 180 mL (60 mL*3) and brine 100 mL (50 mL*2), dried with anhydrous Na₂SO₄, filtered and concentrated under reduced pressure to give a residue. The residue was purified by column chromatography (SiO₂, Petroleum ether/Ethyl acetate=10/1 to 5/1) to give compound O1-tert-butyl O2-[8-(1-hexyl heptoxy)-7,7-dimethyl-8-oxo-octyl] (2S,4S)-4-hydroxypyrrolidine-1,2-dicarboxylate (2.5 g, 4.28 mmol, 38.46% yield) as colorless oil.

### Step 4:

To a solution of O1-tert-butyl O2-[8-(1-hexylheptoxy)-7,7-dimethyl-8-oxo-octyl] (2S,4S)-4-hydroxypyrrolidine-1,2-dicarboxylate (2.5 g, 4.28 mmol, 1 eq) in DCM (15 mL) was added TFA (7.68 g, 67.31 mmol, 5 mL, 15.72 eq). The mixture was stirred at 20° C. for 3 hr. The reaction mixture was adjusted pH to 7 with saturated NaHCO₃ aqueous and extracted with EtOAc 60 mL (20 mL*3), dried over Na₂SO₄, filtered and concentrated under reduced pressure to give a residue. The residue was purified by column chromatography (SiO₂, Petroleum ether/Ethyl acetate=5/1 to 0/1, added 3% NH₃·H₂O) to give compound [8-(1-hexylheptoxy)-7,7-dimethyl-8-oxo-octyl] (2S,4S)-4-hydroxypyrrolidine-2-carboxylate (1.8 g, 3.72 mmol, 86.96% yield) as yellow oil.

### Step 5:

To a solution of [8-(1-hexylheptoxy)-7,7-dimethyl-8-oxo-octyl] (2S,4S)-4-hydroxypyrrolidine-2-carboxylate (1.8 g, 3.72 mmol, 1 eq), undecyl 6-bromo-2,2-dimethyl-hexanoate (1.40 g, 3.72 mmol, 1 eq) in DMF (5 mL) was added K₂CO₃ (2.06 g, 14.88 mmol, 4 eq), KI (617.71 mg, 3.72 mmol, 1 eq). The mixture was stirred at 80° C. for 8 hr. The reaction mixture was diluted with H₂O 20 mL and extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate/NH₃·H₂O=10/1/1 to 1/1/0.5) to give compound [8-(1-hexylheptoxy)-7,7-dimethyl-8-oxo-octyl] (2S,4S)-1-(5,5-dimethyl-6-oxo-6-undec oxy-hexyl)-4-hydroxy-pyrrolidine-2-carboxylate (1.2 g, 1.54 mmol, 37.15% yield) as colorless oil.

¹H NMR (400 MHz, CDCl₃), 4.82-4.85 (m, 1H), 4.26-4.35 (m, 1H), 4.02-4.15 (m, 4H), 3.13-3.31 (m, 2H), 2.38-2.76 (m, 4H), 1.95-2.02 (m, 1H), 1.46-1.68 (m, 14H), 1.23-1.38 (m, 40H), 1.15 (s, 12H), 0.86-0.90 (m, 9H).

### Step 6:

To a solution of [8-(1-hexylheptoxy)-7,7-dimethyl-8-oxo-octyl] (2S,4S)-1-(5,5-dimethyl-6-oxo-6-undecoxy-hexyl)-4-hydroxy-pyrrolidine-2-carboxylate (1.2 g, 1.54 mmol, 1 eq), prop-2-enoyl chloride (696.03 mg, 7.69 mmol, 627.05 μL, 5 eq). DMAP (18.79 mg, 153.81 μmol, 0.1 eq) in DCM (10 mL) was added TEA (1.40 g, 13.84 mmol, 1.93 mL, 9 eq) at 0)° C. The mixture was stirred at 20° C. for 8 hr. The reaction mixture was diluted with H₂O 100 mL and extracted with EtOAc 150 mL (50 mL*3). The combined organic layers were dried over Na₂SO₄, filtered and concentrated under reduced pressure to give a residue. The residue was purified by column chromatography (SiO2, Petroleum ether/Ethyl acetate=1/0 to 95/5) to give compound [8-(1-hexylheptoxy)-7,7-dimethyl-8-oxo-octyl] (2S,4S)-1-(5,5-dimethyl-6-oxo-6-undecoxy-hexyl)-4-prop-2-enoyloxy-pyrrolidine-2-carboxylate (0.7 g, 839.07 μmol, 54.55% yield) as yellow oil.

### Step 7:

A mixture of [8-(1-hexylheptoxy)-7,7-dimethyl-8-oxo-octyl] (2S,4S)-1-(5,5-dimethyl-6-oxo-6-undecoxy-hexyl)-4-prop-2-enoyloxy-pyrrolidine-2-carboxylate (0.7 g, 839.07 μmol, 1 eq) in N-methylmethanamine (2 M/THF, 5.50 mL, 13.12 eq) was degassed and purged with N₂ for 3 times, and then the mixture was stirred at 25° C. for 5 hr under N₂ atmosphere. The reaction mixture was concentrated under reduced pressure to get a residue. The residue was purified by column chromatography (SiO₂, Petroleum ether/Ethyl acetate=10/1 to 4/1, added 3% NH₃·THF). The residue was purified by prep-HPLC (column: Xselect CSH C18 100*30 mm*5 μm; mobile phase: [H₂O (0.04% HCl)-ACN:THF=1:1]; gradient: 30%-70% B over 12.0 min). The residue was adjusted pH to 7 with saturated NaHCO₃ aqueous and extracted with EtOAc 30 mL (10 mL*3). The combined organic layers were dried over Na₂SO₄, filtered and concentrated under reduced pressure to give compound [8-(1-hexylheptoxy)-7,7-dimethyl-8-oxo-octyl] (2S,4S)-4-[3-(dimethylamino)propanoyloxy]-1-(5,5-dimethyl-6-oxo-6-undecoxy-hexyl)pyrrolidine-2-carboxylate (134 mg, 147.82 μmol, 90.25% yield, 97% purity) as colorless oil.

¹H NMR (400 MHz, CDCl₃), 5.18-5.26 (m, 1H), 4.82-4.86 (m, 1H), 4.01-4.13 (m, 4H), 3.09-3.31 (m, 2H), 2.51-2.79 (m, 7H), 2.22-2.40 (m, 7H), 1.98-2.05 (m, 1H), 1.58-1.75 (m, 8H), 1.13-1.46 (m, 58H), 0.86-0.89 (m, 9H), (M+H⁺): 879.7.

HPLC: 11.275 min. LCMS-CAD: (M+H⁺): 879.7 @ 11.508 min.

### 9.21. Synthesis of 2487

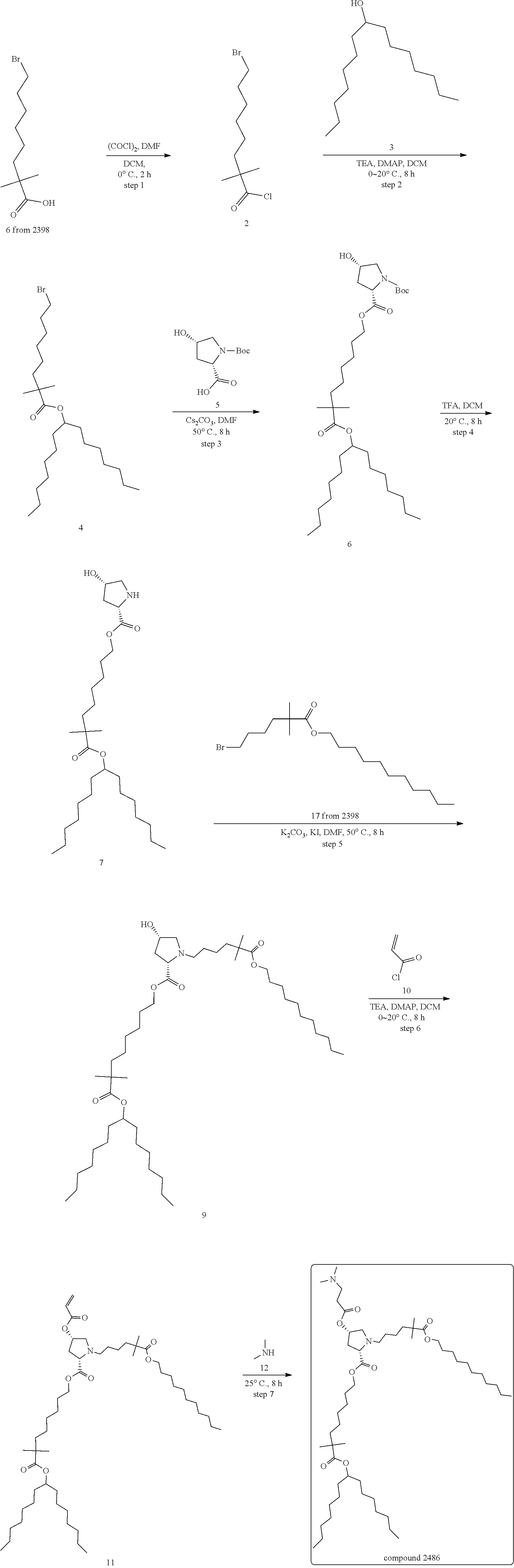

### Step 1:

To a solution of 8-bromo-2,2-dimethyl-octanoic acid (5 g, 19.91 mmol, 1 eq) in DCM (200 mL) was added DMF (72.76 mg, 995.38 μmol, 76.59 μL, 0.05 eq) and oxalyl dichloride (3.03 g, 23.89 mmol, 2.09 mL, 1.2 eq). The mixture was stirred at 0° C. for 2 hr. The mixture was concentrated under reduced pressure to give compound 8-bromo-2,2-dimethyl-octanoyl chloride (10.73 g, crude) as yellow oil.

### Step 2:

To a solution of 8-bromo-2,2-dimethyl-octanoyl chloride (5.24 g, 19.44 mmol, 1.2 eq) in DCM (50 mL) was added TEA (4.92 g, 48.60 mmol, 6.76 mL, 3 eq) and DMAP (989.48 mg, 8.10 mmol, 0.5 eq) and pentadecan-8-ol (3.7 g, 16.20 mmol, 1 eq) at 0° C. The mixture was stirred at 20° C. for 8 hr. The reaction mixture was quenched by addition H₂O 50 mL at 0° C., and then extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=100/1 to 50/1) to give compound 1-heptyloctyl 8-bromo-2,2-dimethyl-octanoate (7 g, 15.17 mmol, 46.81% yield) as yellow oil.

¹H NMR (400 MHz, CDCl₃), 4.80-5.03 (m, 1H), 3.38-3.41 (m, 2H), 1.82-1.88 (m, 2H), 1.50-1.60 (m, 4H), 1.40-1.48 (m, 2H), 1.23-1.38 (m, 28H), 1.15 (s, 6H), 0.86-0.90 (m, 6H).

### Step 3:

To a solution of 1-heptyloctyl 8-bromo-2,2-dimethyl-octanoate (3.83 g, 8.30 mmol, 1.2 eq) in DMF (40 mL) was added Cs₂CO₃ (4.96 g, 15.22 mmol, 2.2 eq) and (2S,4S)-1-tert-butoxy carbonyl-4-hydroxy-pyrrolidine-2-carboxylic acid (1.6 g, 6.92 mmol, 1 eq). The mixture was stirred at 50° C. for 8 hr. The reaction mixture was quenched by addition H₂O 50 mL at 0° C., and then extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=20/1 to 0/1) to give compound O1-tert-butyl O2-[8-(1-heptyloctoxy)-7,7-dimethyl-8-oxo-octyl] (2S,4S)-4-hydroxypyrrolidine-1,2-dicarboxylate (3.8 g, 6.21 mmol, 58.91% yield) as a yellow oil.

### Step 4:

To a solution of O1-tert-butyl O2-18-(1-heptyloctoxy)-7,7-dimethyl-8-oxo-octyl] (2S,4S)-4-hydroxypyrrolidine-1,2-dicarboxylate (3.8 g, 6.21 mmol, 1 eq) in DCM (30 mL) was added TFA (15 mL). The mixture was stirred at 20° C. for 8 hr. The mixture was concentrated under reduced pressure, then adjust pH to 8 with sat. NaHCO₃, extracted with EtOAc 300 mL (100 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound [8-(1-heptyloctoxy)-7,7-dimethyl-8-oxo-octyl] (2S,4S)-4-hydroxypyrrolidine-2-carboxylate (2.3 g, 4.49 mmol, 72.33% yield) as colorless oil.

¹H NMR (400 MHz, CDCl₃), 4.80-4.86 (m, 1H), 4.37-4.42 (m, 1H), 4.12-4.17 (m, 2H), 3.83-3.87 (m, 1H), 2.99-3.17 (m, 2H), 2.50-2.72 (m, 2H), 2.05-2.28 (m, 2H), 1.60-1.68 (m, 2H), 1.45-1.58 (m, 6H), 1.26-1.42 (m, 26H), 1.15 (s, 6H), 0.88 (t, J=6.4 Hz, 6H).

### Step 5:

To a solution of [8-(1-heptyloctoxy)-7,7-dimethyl-8-oxo-octyl] (2S,4S)-4-hydroxypyrrolidine-2-carboxylate (2.3 g, 4.49 mmol, 1 eq) in DMF (30 mL) was added K₂CO₃ (3.11 g, 22.47 mmol, 5 eq) and KI (223.81 mg, 1.35 mmol, 0.3 eq) and undecyl 6-bromo-2,2-dimethyl-hexanoate (1.87 g, 4.94 mmol, 1.1 eq). The mixture was stirred at 50° C. for 8 hr. The reaction mixture was quenched by addition H₂O 60 mL at 0° C. and then extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound [8-(1-heptyloctoxy)-7,7-dimethyl-8-oxo-octyl] (2S,4S)-1-(5,5-dimethyl-6-oxo-6-undecoxy-hexyl)-4-hydroxy-pyrrolidine-2-carboxylate (1.6 g, 1.98 mmol, 44.05% yield) as yellow oil.

### Step 6:

To a solution of [8-(1-heptyloctoxy)-7,7-dimethyl-8-oxo-octyl] (2S,4S)-1-(5,5-dimethyl-6-oxo-6-undecoxy-hexyl)-4-hydroxy-pyrrolidine-2-carboxylate (1.6 g, 1.98 mmol, 1 eq) in DCM (20 mL) was added TEA (2.00 g, 19.80 mmol, 2.76 mL, 10 eq). DMAP (120.92 mg, 989.78 μmol, 0.5 eq) and prop-2-enoyl chloride (895.83 mg, 9.90 mmol, 804.16 μL, 5 eq) at 0° C. The mixture was stirred at 20° C. for 8 hr. The reaction mixture was quenched by addition H₂O 20 mL at 0° C., and then extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound [8-(1-heptyloctoxy)-7,7-dimethyl-8-oxo-octyl] (2S,4S)-1-(5,5-dimethyl-6-oxo-6-undecoxy-hexyl)-4-prop-2-enoyloxy-pyrrolidine-2-carboxylate (800 mg, 927.74 μmol, 46.78% yield) as yellow oil.

¹H NMR (400 MHz, CDCl₃), 5.80-6.43 (m, 3H), 5.22-5.28 (m, 1H), 4.82-4.85 (m, 1H), 4.01-4.12 (m, 4H), 3.12-3.25 (m, 2H), 2.59-2.66 (m, 2H), 2.25-2.35 (m, 1H), 2.02-2.10 (m, 1H), 1.58-1.64 (m, 4H), 1.45-1.56 (m, 10H), 1.22-1.40 (m, 46H), 1.15 (s, 12H), 0.86-0.89 (m, 9H).

### Step 7:

A solution of [8-(1-heptyloctoxy)-7,7-dimethyl-8-oxo-octyl] (2S,4S)-1-(5,5-dimethyl-6-oxo-6-undecoxy-hexyl)-4-prop-2-enoyloxy-pyrrolidine-2-carboxylate (800 mg, 927.74 μmol, 1 eq) in Me₂NH (8 mL). The mixture was stirred at 20° C. for 8 hr. The reaction mixture was quenched by addition H₂O 20 mL at 0° C., and then extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1). The residue was purified by prep-HPLC (column: X-Select CSH Phenyl-Hexyl 100*30 5 u; mobile phase: [H₂O (0.04% HCl)-THF:can=1:3]; gradient: 35%-65% B over 8.0 min). The eluent was freeze-dried. Then adjusted pH to 8 with sat. NaHCO₃, extracted with EtOAc 60 mL (20 mL*3). The combined organic layers were over Na₂SO₄, filtered and concentrated under reduced pressure. The residue was purified by prep-TLC (SiO₂, EA:MeOH=20:1) to give compound [8-(1-heptyloctoxy)-7,7-dimethyl-8-oxo-octyl] (2S,4S)-4-[3-(dimethylamino) propanoyloxy]-1-(5,5-dimethyl-6-oxo-6-undecoxy-hexyl)pyrrolidine-2-carboxylate (23 mg, 24.21 μmol, 28.56% yield, 95.5% purity) as yellow oil.

¹H NMR (400 MHz, CDCl₃), 5.20-5.26 (m, 1H), 4.82-4.85 (m, 1H), 4.02-4.14 (m, 4H), 3.08-3.25 (m, 2H), 2.50-2.76 (m, 7H), 2.03-2.31 (m, 8H), 1.59-1.65 (m, 6H), 1.45-1.55 (m, 10H), 1.27-1.44 (m, 42H), 1.15 (s, 12H), 0.86-0.90 (m, 9H). (M+H⁺): 907.6.

LCMS-CAD: (M+H⁺): 907.6 @ 11.618 min. LCMS-ELSD: (M+H⁺): 907.8 @ 12.200 min.

### 9.22. Synthesis of 2488

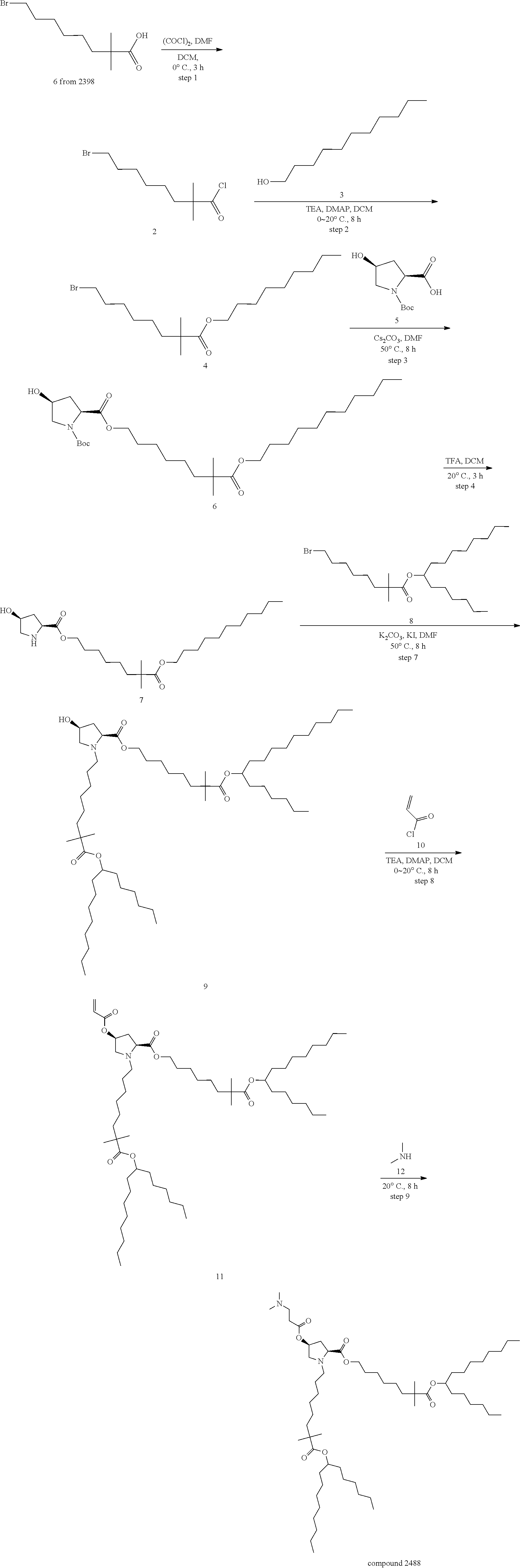

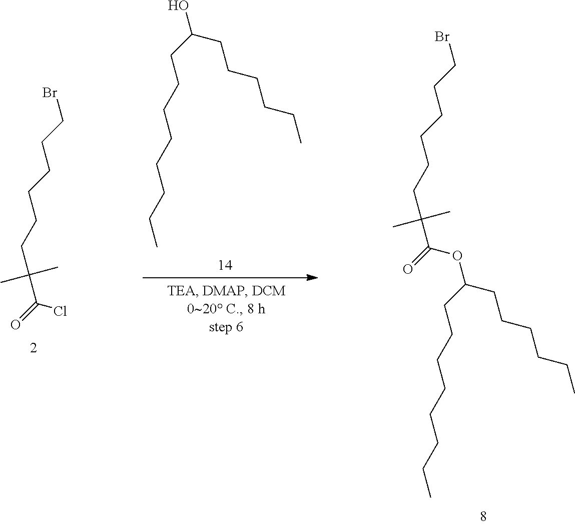 [SMILES: CC(C)(CCCCCCBr)C(=O)Cl ; CCCCCCCCC(O)CCCCCC ; CCCCCCCCC(CCCCCC)OC(=O)C(C)(C)CCCCCCBr]

### Step 1:

To a solution of 8-bromo-2,2-dimethyl-octanoic acid (10 g, 39.82 mmol, 1 eq) in DCM (120 mL) was added DMF (291.02 mg, 3.98 mmol, 306.34 μL, 0.1 eq) and (COCl)₂ (6.06 g, 47.78 mmol, 4.18 mL, 1.2 eq) in sequence. Then the mixture was stirred at 20° C. for 3 hr. The reaction mixture was concentrated under reduced pressure to give compound 8-bromo-2,2-dimethyl-octanoyl chloride (10.7 g, crude) as yellow oil.

### Step 2:

To a solution of undecan-1-ol (6 g, 34.82 mmol, 1 eq) in DCM (100 mL) was added TEA (17.62 g, 174.11 mmol, 24.23 mL, 5 eq) and DMAP (2.13 g, 17.41 mmol, 0.5 eq) and 8-bromo-2,2-dimethyl-octanoyl chloride (10.33 g, 38.30 mmol, 1.1 eq) in DCM (30 mL) in sequence at 0° C. Then the mixture was stirred at 20° C. for 8 hr. The reaction mixture diluted with by addition H₂O 50 mL, and then extracted with EtOAc 45 mL (15 mL*3). The combined organic layers were dried over Na₂SO₄, filtered and concentrated under reduced pressure to give a residue. The residue was purified by column chromatography (SiO₂, Petroleum ether/Ethyl acetate=1/0 to 50/1) to give compound undecyl 8-bromo-2,2-dimethyl-octanoate (9 g, 22.20 mmol, 63.75% yield) as a colorless oil.

¹H NMR (400 MHz, CDCl₃), 4.05 (t, J=6.8 Hz, 2H), 3.40 (t, J=6.8 Hz, 2H), 1.83-1.88 (m, 2H), 1.60-1.68 (m, 2H), 1.49-1.53 (m, 2H), 1.41-1.47 (m, 2H), 1.24-1.40 (m, 20H), 1.16 (s, 6H), 0.89 (t, J=6.4 Hz, 3H).

### Step 3:

To a solution of undecyl 8-bromo-2,2-dimethyl-octanoate (5 g, 12.33 mmol, 1 eq) and (2S,4S)-1-tert-butoxy carbonyl-4-hydroxy-pyrrolidine-2-carboxylic acid (3.42 g, 14.80 mmol, 1.2 eq) in DMF (40 mL) was added Cs₂CO₃ (8.84 g, 27.13 mmol, 2.2 eq). Then the mixture was stirred at 50° C. for 8 hr. The reaction mixture diluted with by addition H₂O 50 mL, and then extracted with EtOAc 45 mL (15 mL*3). The combined organic layers were washed with sat, brine 40 mL (20 mL*2), dried over Na₂SO₄, filtered and concentrated under reduced pressure to give a residue. The residue was purified by column chromatography (SiO₂, Petroleum ether/Ethyl acetate=1/0 to 3/1) to give compound O1-tert-butyl O2-(7,7-dimethyl-8-oxo-8-undecoxy-octyl) (2S,4S)-4-hydroxypyrrolidine-1,2-dicarboxylate (3.5 g, 6.30 mmol, 51.07% yield) as a yellow oil.

### Step 4:

To a solution of O1-tert-butyl O2-(7,7-dimethyl-8-oxo-8-undecoxy-octyl) (2S,4S)-4-hydroxy pyrrolidine-1,2-dicarboxylate (3.50 g, 6.30 mmol, 1 eq) in DCM (20 mL) was added TFA (15.35 g, 134.62 mmol, 10 mL, 21.38 eq). Then the mixture was stirred at 20° C. for 3 hr. The reaction mixture was adjusted pH to 8 with sat. NaHCO₃, and then extracted EtOAc 90 mL (30 mL*3). The combined organic layers were dried over Na₂SO₄, filtered and concentrated under reduced pressure to give compound (7,7-dimethyl-8-oxo-8-undecoxy-octyl) (2S,4S)-4-hydroxypyrrolidine-2-carboxylate (2.5 g, 5.49 mmol, 87.12% yield) as a yellow oil.

¹H NMR (400 MHz, CDCl₃), 4.55 (s, 1H), 4.32-4.38 (m, 1H), 4.10-4.22 (m, 3H), 4.04 (t, J=6.4 Hz, 2H), 3.56 (d, J=12.0 Hz, 1H), 3.24-3.28 (m, 1H), 2.38-2.45 (m, 2H), 1.61-1.66 (m, 4H), 1.46-1.55 (m, 2H), 1.24-1.38 (m, 22H), 1.15 (s, 6H), 0.88 (t, J=6.4 Hz, 3H).

### Step 6:

To a solution of 8-bromo-2,2-dimethyl-octanoyl chloride (10 g, 37.09 mmol, 1 eq), TEA (18.77 g, 185.46 mmol, 25.81 mL, 5 eq). DMAP (906.29 mg, 7.42 mmol, 0.2 eq) in DCM (100 mL) was added pentadecan-7-ol (8.47 g, 37.09 mmol, 1 eq) at 0° C. The mixture was stirred at 20° C. for 8 hr. The reaction mixture was diluted with H₂O 200 mL and extracted with EtOAc 600 mL (200 mL*3). The combined organic layers were dried over Na₂SO₄, filtered and concentrated under reduced pressure to give a residue. The residue was purified by column chromatography (SiO₂, Petroleum ether/Ethyl acetate=1/0)) to give compound 1-hexylnonyl 8-bromo-2,2-dimethyl-octanoate (8.8 g, 19.07 mmol, 51.76% yield) as colorless oil.

¹H NMR (400 MHz, CDCl₃), 4.81-4.87 (m, 1H), 3.40 (t, J=6.8 Hz, 2H), 1.81-1.88 (m, 2H), 1.45-1.56 (m, 8H), 1.17-1.39 (m, 24H), 1.16 (s, 6H), 0.88 (t, J=6.4 Hz, 6H).

### Step 7:

To a solution of (7,7-dimethyl-8-oxo-8-undecoxy-octyl) (2S,4S)-4-hydroxypyrrolidine-2-carboxylate (1.50 g, 3.29 mmol, 1 eq) and 1-hexylnonyl 8-bromo-2,2-dimethyl-octanoate (1.82 g, 3.95 mmol, 1.2 eq) in DMF (30 mL) was added K₂CO₃ (1.36 g, 9.88 mmol, 3 eq) and KI (1.09 g, 6.58 mmol, 2 eq) in sequence. Then the mixture was stirred at 50° C. for 8 hr. The reaction mixture diluted with by addition H₂O 50 mL, and then extracted with EtOAc 45 mL (15 mL*3). The combined organic layers were washed with sat, brine 40 mL (20 mL*2), dried over Na₂SO₄, filtered and concentrated under reduced pressure to give a residue. The residue was purified by column chromatography (SiO₂, Petroleum ether/Ethyl acetate=1/0 to 5/1) to give compound (7,7-dimethyl-8-oxo-8-undecoxy-octyl) (2S,4S)-1-[8-(1-hexylnonoxy)-7,7-dimethyl-8-oxo-octyl]-4-hydroxy-pyrrolidine-2-carboxylate (1.3 g, 1.55 mmol, 47.22% yield) as colorless oil.

### Step 8:

To a solution of (7,7-dimethyl-8-oxo-8-undecoxy-octyl) (2S,4S)-1-[8-(1-hexylnonoxy)-7,7-dimethyl-8-oxo-octyl]-4-hydroxy-pyrrolidine-2-carboxylate (1.30 g, 1.55 mmol, 1 eq) in DCM (10 mL) was added TEA (1.57 g, 15.54 mmol, 2.16 mL, 10 eq) and DMAP (94.95 mg, 777.22 μmol, 0.5 eq) and prop-2-enoyl chloride (703.45 mg, 7.77 mmol, 631.46 μL, 5 eq) in DCM (5 mL) in sequence at 0° C. Then the mixture was stirred at 20° C. for 8 hr. The reaction mixture diluted with by addition H₂O 30 mL, and then extracted with EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate=1/0 to 10/1) to give compound (7,7-dimethyl-8-oxo-8-undecoxy-octyl) (2S,4S)-1-[8-(1-hexylnonoxy)-7,7-dimethyl-8-oxo-octyl]-4-prop-2-enoyloxy-pyrrolidine-2-carboxylate (600 mg, 673.88 μmol, 43.35% yield) as a colorless oil.

¹H NMR (400 MHz, CDCl₃), 6.39-6.44 (m, 1H), 6.10-6.17 (m, 1H), 5.80-5.83 (m, 1H), 5.25-5.27 (m, 1H), 4.81-4.84 (m, 1H), 4.10-4.15 (m, 4H), 3.28-3.30 (m, 1H), 3.13 (t, J=8.0 Hz, 1H), 2.73-2.75 (m, 1H), 2.60-2.65 (m, 2H), 2.25-2.34 (m, 1H), 2.06-2.12 (m, 1H), 1.58-1.66 (m, 8H), 1.45-1.55 (m, 8H), 1.25-1.42 (m, 46H), 1.15 (d, J=4.4 Hz, 12H), 0.86-0.89 (m, 9H).

### Step 9:

A solution of (7,7-dimethyl-8-oxo-8-undecoxy-octyl) (2S,4S)-1-[8-(1-hexylnonoxy)-7,7-dimethyl-8-oxo-octyl]-4-prop-2-enoyloxy-pyrrolidine-2-carboxylate (600.00 mg, 673.88 μmol, 1 eq) in N-methylmethanamine (2 M/THF, 6 mL, 17.81 eq) was stirred at 20° C. for 8 hr. The reaction mixture was concentrated under reduced pressure to remove solvent. The residue was purified by column chromatography (SiO₂, Petroleum ether/Ethyl acetate=1/0 to 5/1). The reaction mixture diluted with by addition PE 20 mL, and then extracted with ACN 30 mL (15 mL*2). The combined PE layers were concentrated under reduced pressure to give a residue. The residue was purified by prep-TLC (SiO₂, Petroleum ether/Ethyl acetate=0.1) and prep-HPLC (column: X-Select CSH Phenyl-Hexyl 100*30 5 u; mobile phase: [H₂O (0.04% HCl)-THF:ACN=1:3]; gradient: 30%-75% B over 8.0 min). The eluent was adjusted pH to 8 with sat. NaHCO₃, and then extracted EtOAc 15 mL (5 mL*3). The combined organic layers were dried over Na₂SO₄, filtered and concentrated under reduced pressure to give a residue. Then diluted with by addition PE 20 mL and extracted with ACN 30 mL (15 mL*2). The combined PE layers were concentrated under reduced pressure and purified by column chromatography (SiO₂, Petroleum ether/Ethyl acetate=10/1 to 0/1, added 0.5% NH₃·H₂O) to give compound (7,7-dimethyl-8-oxo-8-undecoxy-octyl) (2S,4S)-4-[3-(dimethylamino) propanoyloxy]-1-[8-(1-hexylnonoxy)-7,7-dimethyl-8-oxo-octyl]pyrrolidine-2-carboxylate (0.016 g, 16.08 μmol, 41.78% yield, 94% purity) as colorless oil.

¹H NMR (400 MHz, CDCl₃), 5.18-5.24 (m, 1H), 4.81-4.84 (m, 1H), 4.03-4.14 (m, 4H), 3.09-3.26 (m, 2H), 2.80-3.05 (m, 2H), 2.62-2.78 (m, 3H), 2.57-2.61 (m, 3H), 2.27-2.48 (m, 6H), 2.00-2.07 (m, 1H), 1.58-1.68 (m, 6H), 1.48-1.56 (m, 12H), 1.26-1.42 (m, 44H), 1.15 (d, J=5.2 Hz, 12H), 0.86-0.89 (m, 9H). (M+H⁺): 935.7.

LCMS-CAD: (M+H⁺): 935.7 @ 10.445 min. LCMS-ELSD: (M+H⁺): 935.8 @ 10.999 min.

### 9.23. Synthesis of 2498

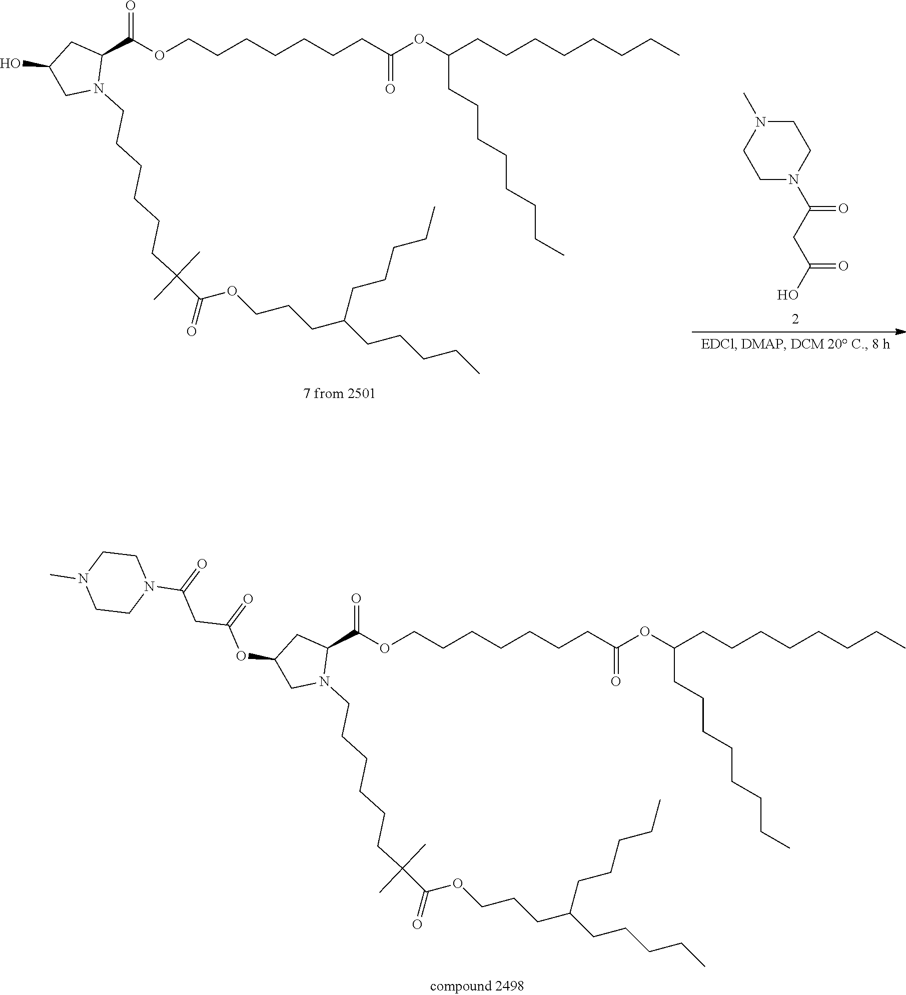

To a solution of [8-(1-octylnonoxy)-8-oxo-octyl] (2S,4S)-1-[7,7-dimethyl-8-oxo-8-(4-pentylnonoxy)octyl]-4-hydroxy-pyrrolidine-2-carboxylate (500 mg, 569.22 μmol, 1 eq) and 3-(4-methylpiperazin-1-yl)-3-oxo-propanoic acid (105.99 mg, 569.22 μmol, 1 eq) in DCM (5 mL) was added EDCI (163.68 mg, 853.83 μmol, 1.5 eq) and DMAP (34.77 mg, 284.61 μmol, 0.5 eq) in sequence. Then the mixture was stirred at 20° C. for 8 hr. The reaction mixture diluted with by addition H₂O 50 mL, and then extracted with EtOAc 45 mL (15 mL*3). The combined organic layers were dried over Na₂SO₄, filtered and concentrated under reduced pressure to give a residue. The residue was purified by column chromatography (SiO₂, Ethyl acetate:MeOH=1/0 to ½, added 3% NH₃·THF). The crude product was purified by reversed-phase HPLC ([H₂O (0.04% HCl)-ACN]; gradient: 25%-70% B over 12.0 min). Then the fraction was freeze-dried to obtain the product. The product was adjusted pH to 8 with sat. NaHCO₃ and extracted with EtOAc (10 mL*3). The combined organic layers were dried over Na₂SO₄, filtered and concentrated under reduced pressure to give a residue. The residue was purified by prep-TLC (Petroleum ether:Ethyl acetate=0.1). The residue was purified by prep-HPLC ([H₂O (0.04% HCl)-ACN:THF=1:1]) and adjusted pH to 8 with sat. NaHCO₃ and extracted with EtOAc (10 mL*3). The combined organic layers were dried over Na₂SO₄, filtered and concentrated under reduced pressure to give compound [8-(1-octylnonoxy)-8-oxo-octyl] (2S,4S)-1-[7,7-dimethyl-8-oxo-8-(4-pentylnonoxy)octyl]-4-[3-(4-methylpiperazin-1-yl)-3-oxo-propanoyl]oxy-pyrrolidine-2-carboxylate (19 mg, 18.15 μmol, 21.11% yield) as colorless oil.

¹H NMR (400 MHz, CDCl₃), 5.21-5.29 (m, 1H), 4.83-4.90 (m, 1H), 4.11 (t, J=6.8 Hz, 2H), 4.03 (t, J=6.4 Hz, 2H), 3.42-3.80 (m, 5H), 3.27 (d, J=11.2 Hz, 1H), 3.10 (t, J=8.4 Hz, 1H), 2.22-2.80 (m, 12H), 2.02-2.10 (m, 1H), 1.57-1.68 (m, 10H), 1.43-1.56 (m, 8H), 1.21-1.39 (m, 53H), 1.15 (s, 6H), 0.86-0.91 (m, 12H), (M+H⁺): 1046.7.

LCMS: (M+H⁺): 1046.7 @ 8.980 min. LCMS: (M+H⁺): 1046.9 @ 9.334 min.

### 9.24. Synthesis of 2500

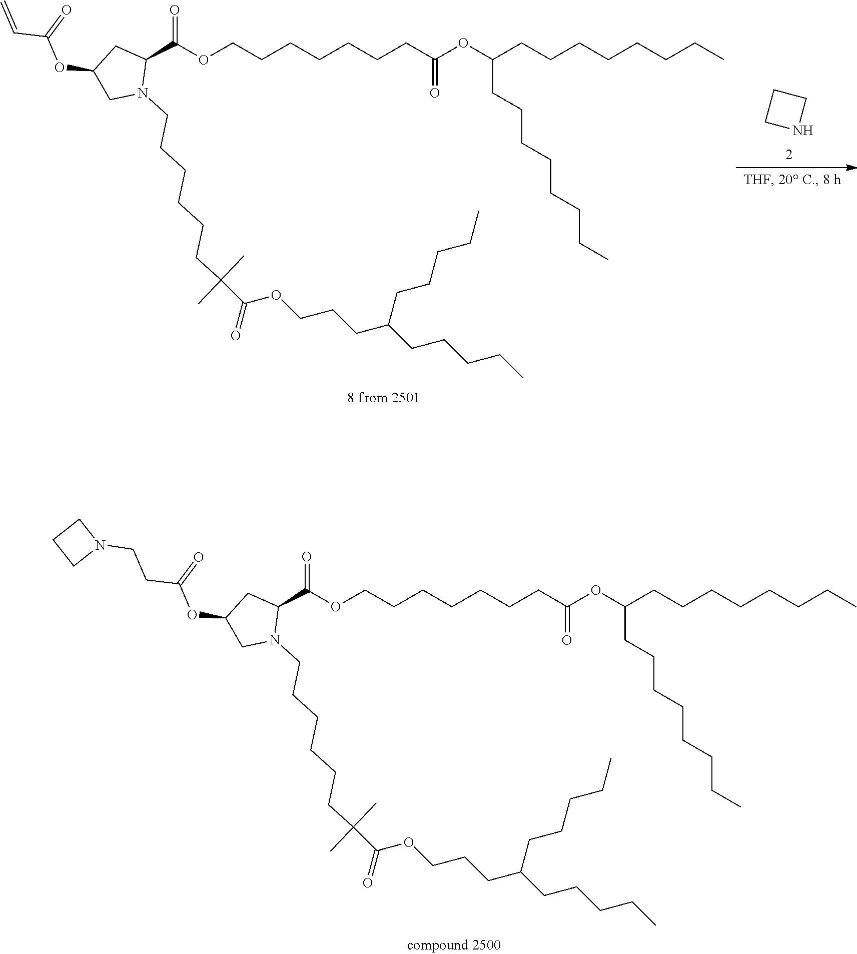

A mixture of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxylate (0.5 g, 536.23 μmol, 1 eq), azetidine (153.08 mg, 2.68 mmol, 180.94 μL, 5 eq) in THF (5 mL) was degassed and purged with N₂ for 3 times, and then the mixture was stirred at 20° C. for 8 hr under N₂ atmosphere. The reaction mixture was concentrated under reduced pressure to get a residue. The residue was purified by column chromatography (SiO₂, Petroleum ether/Ethyl acetate=10/1 to 3/1, added 3% NH₃·THF) to give compound [8-(1-octylnonoxy)-8-oxo-octyl] (2S,4S)-4-[3-(azetidin-1-yl)propanoyloxy]-1-[7,7-di methyl-8-oxo-8-(4-pentylnonoxy)octyl]pyrrolidine-2-carboxylate (0.08 g, 76.80 μmol, 38.00% yield, 95% purity) as yellow oil.

¹H NMR (400 MHz, CDCl₃), 5.18-5.26 (m, 1H), 4.85-4.88 (m, 1H), 4.01-4.14 (m, 4H), 3.07-3.26 (m, 6H), 2.56-2.70 (m, 5H), 2.26-2.37 (m, 5H), 2.04-2.08 (m, 3H), 1.58-1.65 (m, 8H), 1.24-1.51 (m, 61H), 1.15 (s, 6H), 0.86-0.90 (m, 12H). (M+H⁺): 989.7.

LCMS-CAD: (M+H⁺): 989.7 @ 9.591 min. LCMS-ELSD: (M+H⁺): 989.5 @ 9.776 min.

### 9.25. Synthesis of 2501

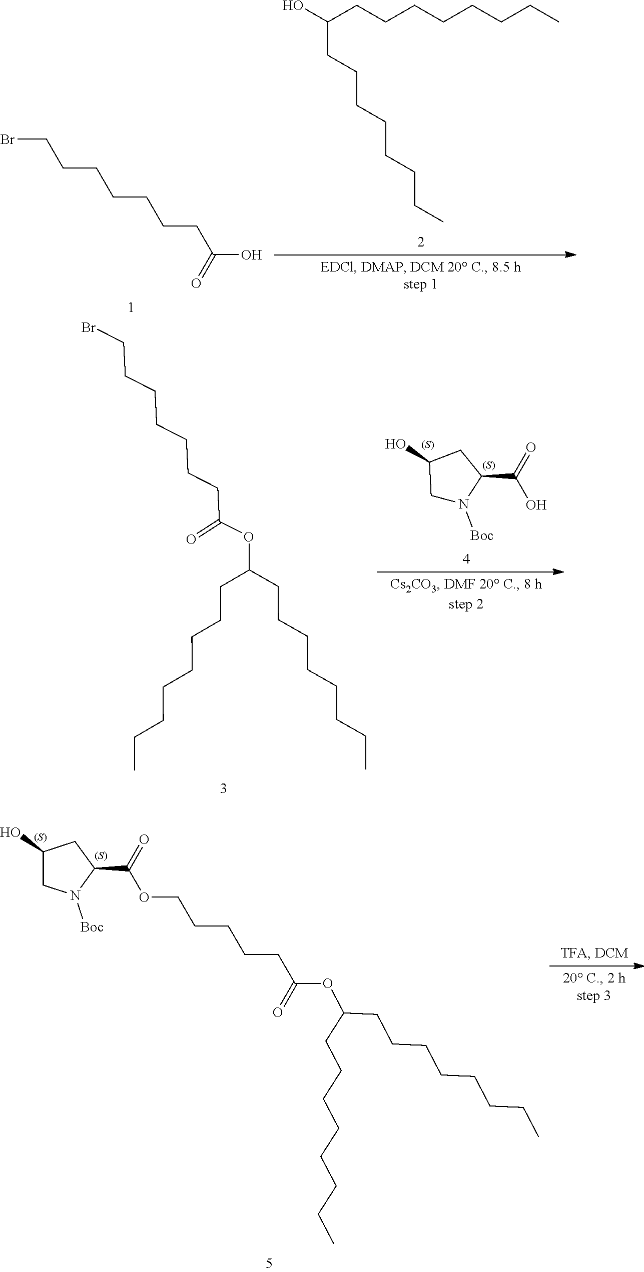

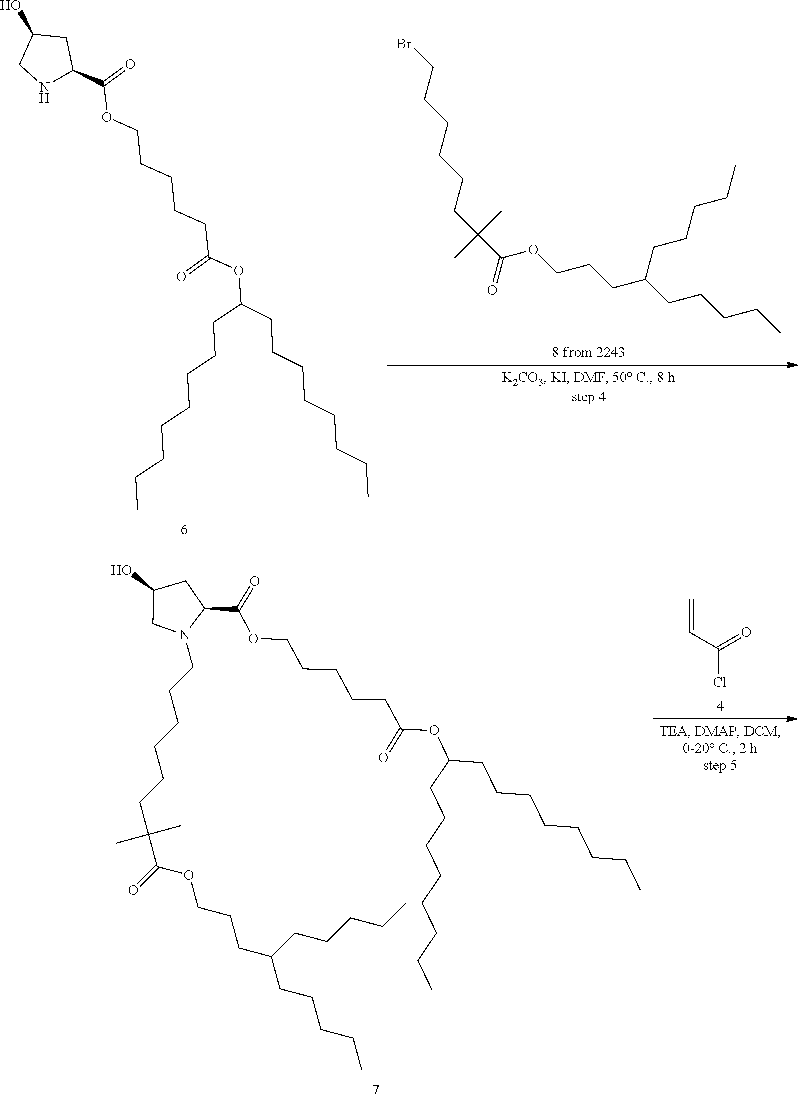

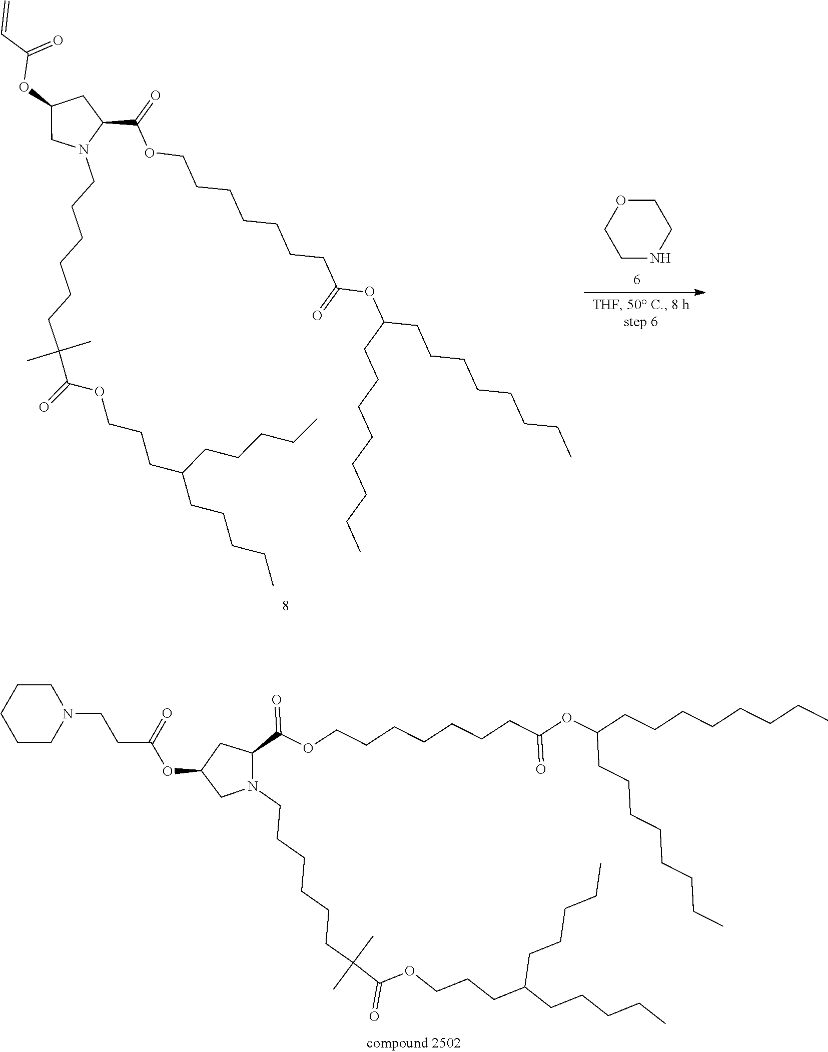 [SMILES: C=CC(=O)O[C@H]1C[C@@H](C(=O)OCCCCCCCC(=O)OC(CCCCCCCC)CCCCCCCC)N(CCCCCCC(C)(C)C(=O)OCCCC(CCCCC)CCCCC)C1 ; C1COCCN1 ; CCCCCCCCC(CCCCCCCC)OC(=O)CCCCCCCOC(=O)[C@@H]1C[C@H](OC(=O)CCN2CCCCC2)CN1CCCCCCC(C)(C)C(=O)OCCCC(CCCCC)CCCCC]

### Step 1:

To a solution of 8-bromooctanoic acid (10 g, 44.82 mmol, 2.40 eq) in DCM (70 mL) was added EDCI (4.65 g, 24.28 mmol, 1.3 eq) and DMAP (2.97 g, 24.28 mmol, 1.3 eq). The mixture was stirred at 20° C. for 0.5 h. To mixture was added heptadecan-9-ol (4.79 g, 18.68 mmol, 1 eq) and stirred at 20° C. for 8 h. The reaction mixture was concentrated under reduced pressure, and then diluted with H₂O 200 mL and extracted with EtOAc 900 mL (300 mL*3). The combined organic layers were washed with brine 200 mL, dried over Na₂SO₄, filtered and concentrated under reduced pressure to give a residue. The residue was purified by column chromatography (SiO₂, Petroleum ether/Ethyl acetate=1/0 to 10/1) to give compound 1-octylnonyl 8-bromooctanoate (72 g, 78.00 mmol, 69.61% yield) as colorless oil.

### Step 2:

To a solution of 1-octylnonyl 8-bromooctanoate (40.72 g, 88.22 mmol, 1.2 eq) in DMF (600 mL) was added Cs₂CO₃ (52.70 g, 161.73 mmol, 2.2 eq) and (2S,4S)-1-tert-butoxycarbonyl-4-hydroxy-pyrrolidine-2-carboxylic acid (17 g, 73.52 mmol, 1 eq). The mixture was stirred at 20° C. for 8 hr. The reaction mixture was quenched by addition H₂O 400 mL at 0° C. and then extracted with EtOAc 300 mL (100 mL*3). The combined organic layers were dried over Na₂SO₄, filtered and concentrated under reduced pressure to give a residue. The residue was purified by column chromatography (SiO₂, Petroleum ether/Ethyl acetate=1/0 to 10/1) to give compound O1-tert-butyl O2-[8-(1-octylnonoxy)-8-oxo-octyl] (2S,4S)-4-hydroxypyrrolidine-1,2-dicarboxylate (65 g, 106.23 mmol, 72.25% yield) as colorless oil.

¹H NMR (400 MHz, CDCl₃), 4.86 (t, J=6.4 Hz, 1H), 4.13-4.37 (m, 4H), 3.30-3.69 (m, 3H), 2.28-2.35 (m, 3H), 2.05-2.15 (m, 1H), 1.63-1.66 (m, 6H), 1.46-1.51 (m, 13H), 1.26-1.43 (m, 28H), 0.88 (t, J=6.8 Hz, 6H) 20)

### Step 3:

To a solution of O1-tert-butyl O2-[8-(1-octylnonoxy)-8-oxo-octyl] (2S,4S)-4-hydroxypyrrolidine-1,2-dicarboxylate (10 g, 16.34 mmol, 1 eq) in DCM (140 mL) was added TFA (70 mL). The mixture was stirred at 20° C. for 2 hr. The mixture was concentrated under reduced pressure, then adjust pH to 8 with sat. NaHCO₃, extracted with EtOAc 300 mL (100 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound [8-(1-octylnonoxy)-8-oxo-octyl] (2S,4S)-4-hydroxypyrrolidine-2-carboxylate (13 g, 25.40 mmol, 77.72% yield) as colorless oil.

### Step 4:

To a mixture of [8-(1-octylnonoxy)-8-oxo-octyl] (2S,4S)-4-hydroxypyrrolidine-2-carboxylate (10 g, 19.54 mmol, 1 eq) in DMF (100 mL) was added K₂CO₃ (8.10 g, 58.62 mmol, 3 eq) and KI (648.73 mg, 3.91 mmol, 0.2 eq), then 4-pentylnonyl 8-bromo-2,2-dimethyl-octanoate (10.49 g, 23.45 mmol, 1.2 eq) was added into the mixture. The mixture was stirred at 50° C., for 8 h. The mixture was added into H₂O (200 mL), extracted with EtOAc (100 mL*3), combined organic layer was washed with brine (100 mL*2), dried over Na₂SO₄, filtered and the filtrate was concentrated under reduced pressure. The residue was purified by column chromatography (SiO₂, Petroleum ether/Ethyl acetate=100/1 to 10/1) to give compound [8-(1-octylnonoxy)-8-oxo-octyl] (2S,4S)-1-[7,7-dimethyl-8-oxo-8-(4-pentylnonoxy)octyl]-4-hydroxy-pyrrolidine-2-carboxylate (11 g, 12.52 mmol, 64.09% yield) as colorless oil.

### Step 5:

To a solution of [8-(1-octylnonoxy)-8-oxo-octyl] (2S,4S)-1-[7,7-dimethyl-8-oxo-8-(4-pentylnonoxy)octyl]-4-hydroxy-pyrrolidine-2-carboxylate (10 g, 11.38 mmol, 1 eq) and TEA (5.76 g, 56.92 mmol, 7.92 mL, 5 eq) in DCM (150 mL) was added DMAP (278.16 mg, 2.28 mmol, 0.2 eq) and prop-2-enoyl chloride (3.09 g, 34.15 mmol, 2.77 mL, 3 eq) under N₂ at 0° C., and then the mixture was stirred at 20° C. for 2 h. The mixture was added into sat. NaHCO₃ (200 mL), extracted with EtOAc (100 mL*3), organic layer was washed with brine (100 mL*2), dried over Na₂SO₄, filtered and the filtrate was concentrated under reduced pressure. The residue was purified by column chromatography (SiO₂, Petroleum ether/Ethyl acetate=10/1 to 0/1, added 5% NH₃·THF) to give compound [8-(1-octylnonoxy)-8-oxo-octyl] (2S,4S)-1-[7,7-dimethyl-8-oxo-8-(4-pentylnonoxy)octyl]-4-prop-2-enoyloxypyrrolidine-2-carboxylate (7.2 g, 7.72 mmol, 67.83% yield, -purity) as yellow oil.

¹H NMR (400 MHz, CDCl₃), 6.41 (d, J=17.2 Hz, 1H), 6.11-6.17 (m, 1H), 5.82 (d, J=10.0 Hz, 1H), 5.26 (s, 1H), 4.85-4.88 (m, 1H), 4.01-4.13 (m, 4H), 3.29 (d, J=11.6 Hz, 1H), 3.14 (t, J=8.4 Hz, 1H), 2.60-2.76 (m, 2H), 2.28-2.30 (m, 3H), 2.04-2.11 (m, 1H), 1.60-1.64 (m, 6H), 1.42-1.51 (m, 8H), 1.24-1.41 (m, 54H), 1.15 (s, 6H), 0.86-0.90 (m, 12H).

### Step 6:

To a solution of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxylate (500 mg, 536.23 μmol, 1 eq) in THF (5 mL) was added morpholine (93.43 mg, 1.07 mmol, 94.38 μL, 2 eq). The mixture was stirred at 50° C. for 8 hr. The mixture was concentrated under reduced pressure to give a residue. The residue was purified by column chromatography (SiO₂, Petroleum ether/Ethyl acetate=10/1 to 0/1) to give compound [8-(1-octylnonoxy)-8-oxo-octyl] (2S,4S)-1-[7,7-dimethyl-8-oxo-8-(4-pentylnonoxy)octyl]-4-(3-morpholinopropanoyloxy)pyrrolidine-2-carboxylate (194 mg, 187.80 μmol, 35.02% yield, 98.7% purity) as a yellow oil.

¹H NMR (400 MHz, CDCl₃), 5.18-5.21 (m, 1H), 4.83-4.88 (m, 1H), 4.01-4.13 (m, 4H), 3.69 (s, 4H), 3.08-3.26 (m, 2H), 2.46-2.75 (m, 11H), 2.28 (t, J=7.6 Hz, 3H), 2.01-2.06 (m, 1H), 1.56-1.66 (m, 6H), 1.47-1.55 (m, 8H), 1.24-1.38 (m, 55H), 1.15 (s, 6H), 0.88-0.90 (m, 12H). (M+H⁺): 1019.7. LCMS-CAD: (M+H⁺): 1019.7 @ 11.941 min. LCMS-ELSD: (M+H⁺): 1019.9 @ 13.756 min.

### 9.26. Synthesis of 2502

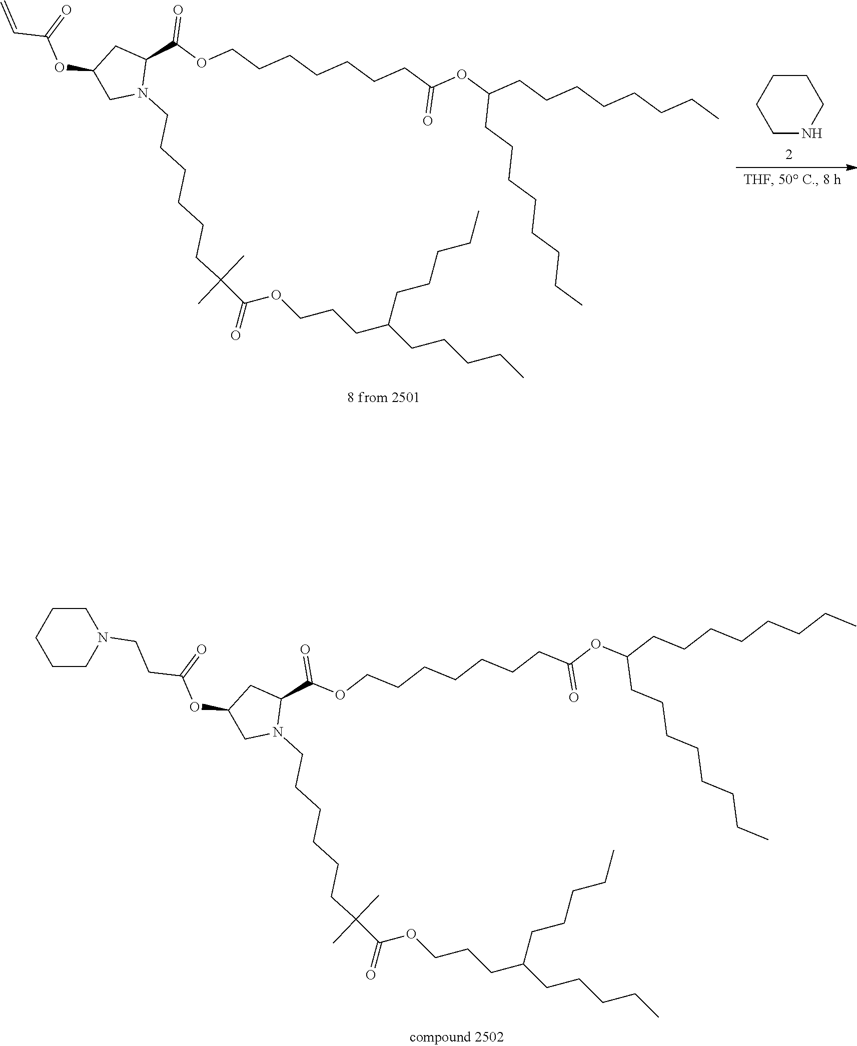

To a solution of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxylate (500 mg, 536.23 μmol, 1 eq) in THF (5 mL) was added piperidine (91.32 mg, 1.07 mmol, 105.91 μL, 2 eq). The mixture was stirred at 50° C. for 8 hr. The mixture was concentrated under reduced pressure to give a residue. The residue was purified by column chromatography (SiO₂, Petroleum ether/Ethyl acetate=10/1 to 0/1). The reaction mixture was diluted with PE 10 mL and extracted with ACN 20 mL (10 mL*2). The PE layers were concentrated under reduced pressure to give compound [8-(1-octylnonoxy)-8-oxo-octyl] (2S,4S)-1-[7,7-dimethyl-8-oxo-8-(4-pentylnonoxy)octyl]-4-[3-(1-piperidyl)propanoyloxy]pyrrolidine-2-carboxylate (45 mg, 42.41 μmol, 43.16% yield, 95.9% purity) as a yellow oil.

¹H NMR (400 MHz, CDCl₃), 5.17-5.22 (m, 1H), 4.85-4.88 (m, 1H), 4.11-4.13 (m, 2H), 4.03 (t, J=6.4 Hz, 2H), 3.24 (d, J=11.2 Hz, 1H), 3.10 (t, J=8.0 Hz, 1H), 2.26-2.74 (m, 12H), 2.00-2.05 (m, 1H), 1.58-1.64 (m, 8H), 1.47-1.51 (m, 8H), 1.24-1.38 (m, 61H), 1.15 (s, 6H), 0.88-0.90 (m, 12H). (M+H⁺): 1017.7. LCMS-CAD: (M+H⁺): 1017.7 @ 9.739 min.

LCMS-ELSD: (M+H⁺): 1017.9 @ 10.106 min,

### 9.27. Synthesis of 2503

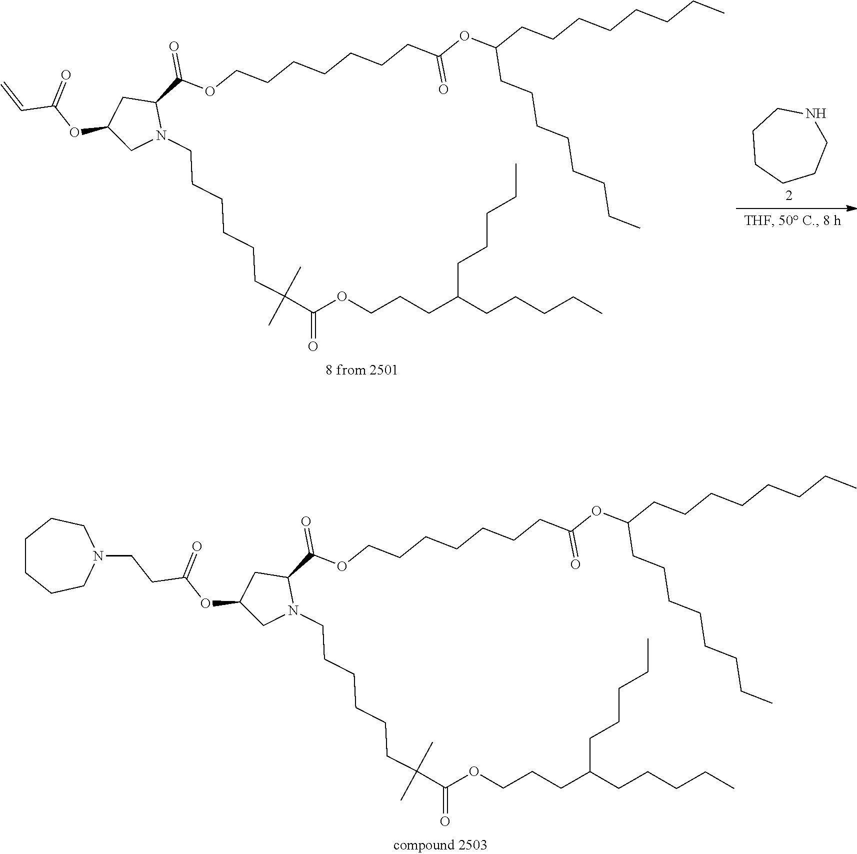 [SMILES: C=CC(=O)O[C@H]1C[C@@H](C(=O)OCCCCCCCC(=O)OC(CCCCCCCC)CCCCCCCC)N(CCCCCCC(C)(C)C(=O)OCCCC(CCCCC)CCCCC)C1 ; C1CCCNCC1 ; CCCCCCCCC(CCCCCCCC)OC(=O)CCCCCCCOC(=O)[C@@H]1C[C@H](OC(=O)CCN2CCCCCC2)CN1CCCCCCC(C)(C)C(=O)OCCCC(CCCCC)CCCCC]

To a solution of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxylate (500 mg, 536.23 μmol, 1 eq) in THF (5 mL) was added azepane (265.90 mg, 2.68 mmol, 302.16 μL, 5 eq). The mixture was stirred at 50° C. for 8 hr. The reaction mixture was quenched by addition H₂O 60 mL at 0° C., and stirred for 0.5 h under N₂, and then extracted with EtOAc 150 mL (50 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1) to give compound [8-(1-octylnonoxy)-8-oxo-octyl] (2S,4S)-4-[3-(azepan-1-yl)propanoyl oxy]-1-[7,7-dimethyl-8-oxo-8-(4-pentylnonoxy)octyl]pyrrolidine-2-carboxylate (132 mg, 127.95 μmol, 23.86% yield) as yellow oil.

¹H NMR (400 MHz, CDCl₃), 5.13-5.22 (m, 1H), 4.85-4.88 (m, 1H), 4.01-4.14 (m, 4H), 2.55-3.26 (m, 13H), 2.25-2.30 (m, 3H), 1.99-2.05 (m, 1H), 1.58-1.65 (m, 12H), 1.47-1.55 (m, 8H), 1.23-1.41 (m, 57H), 1.15 (s, 6H), 0.88-0.90 (m, 12H). (M+H⁺): 1031.8.

LCMS-CAD: (M+H⁺): 1031.8 @ 12.110 min.

LCMS-ELSD: (M+H⁺): 1031.8 @ 12.261 min.

### 9.28. Synthesis of 2504

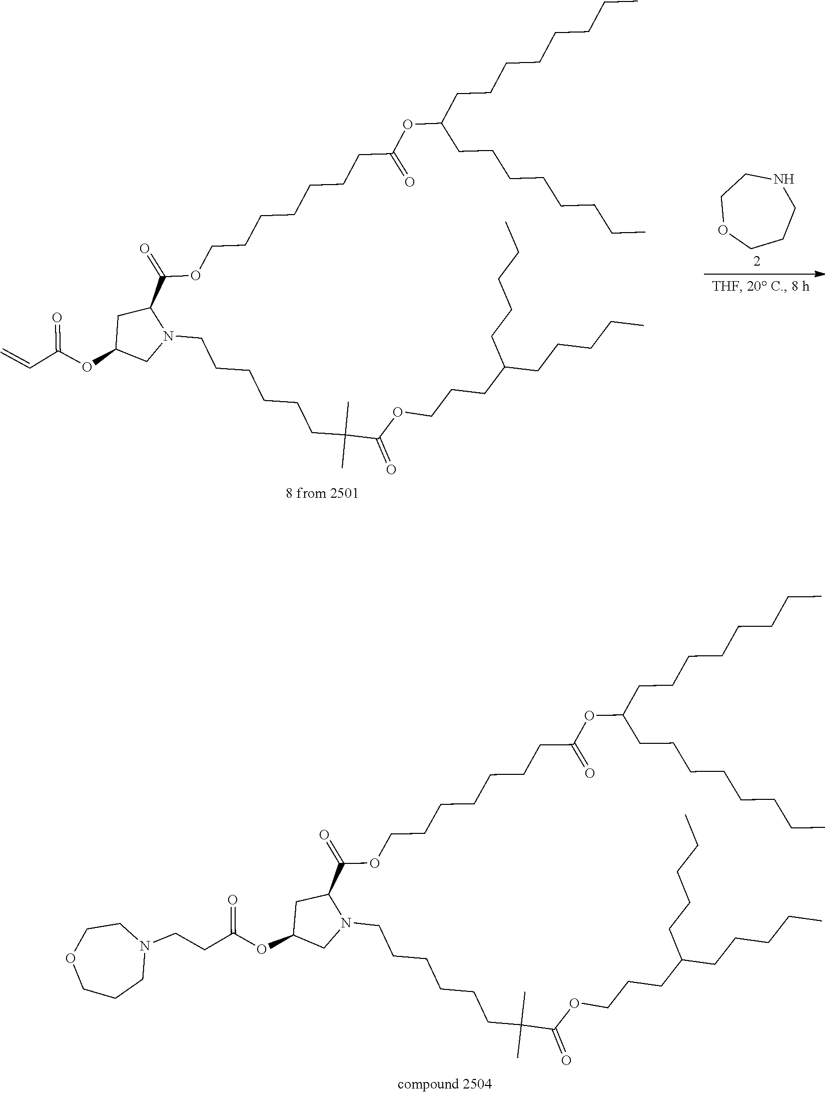 [SMILES: C=CC(=O)O[C@H]1C[C@@H](C(=O)OCCCCCCCC(=O)OC(CCCCCCCC)CCCCCCCC)N(CCCCCCC(C)(C)C(=O)OCCCC(CCCCC)CCCCC)C1 ; C1CNCCOC1 ; CCCCCCCCC(CCCCCCCC)OC(=O)CCCCCCCOC(=O)[C@@H]1C[C@H](OC(=O)CCN2CCCOCC2)CN1CCCCCCC(C)(C)C(=O)OCCCC(CCCCC)CCCCC]

A mixture of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxylate (0.5 g, 536.23 μmol, 1 eq), 1,4-oxazepane (189.83 mg, 1.88 mmol, 3.5 eq) in THF (5 mL) was degassed and purged with N₂ for 3 times and then the mixture was stirred at 20° C. for 8 hr under N₂ atmosphere. The reaction mixture was concentrated under reduced pressure to get a residue. The residue was purified by column chromatography (SiO₂, Petroleum ether/Ethyl acetate=10/1 to 3/1, added 3% NH₃·THF) to give compound [8-(1-octylnonoxy)-8-oxo-octyl] (2S,4S)-1-[7,7-dimethyl-8-oxo-8-(4-pentylnonoxy)octyl]-4-[3-(1,4-oxazepan-4-yl)propanoyloxy]pyrrolidine-2-carboxylate (0.184 g, 178.02 μmol, 92.00% yield) as yellow oil.

¹H NMR (400 MHz, CDCl₃), 5.14-5.22 (m, 1H), 4.85-4.88 (m, 1H), 4.01-4.14 (m, 4H), 3.80 (L, J=5.2 Hz, 4H), 2.57-3.26 (m, 13H), 2.28 (1, J=7.6 Hz, 3H), 1.75-2.15 (m, 3H), 1.62-1.71 (m, 4H), 1.48-1.58 (m, 8H), 1.24-1.42 (m, 57H), 1.15 (s, 6H), 0.86-0.90 (m, 12H). (M+H⁺): 1033.7. LCMS-CAD: (M+H⁺): 1033.7 @ 11.926 min.

LCMS-ELSD: (M+H⁺): 1033.9 @ 10.031 min.

### 9.29. Synthesis of 2505

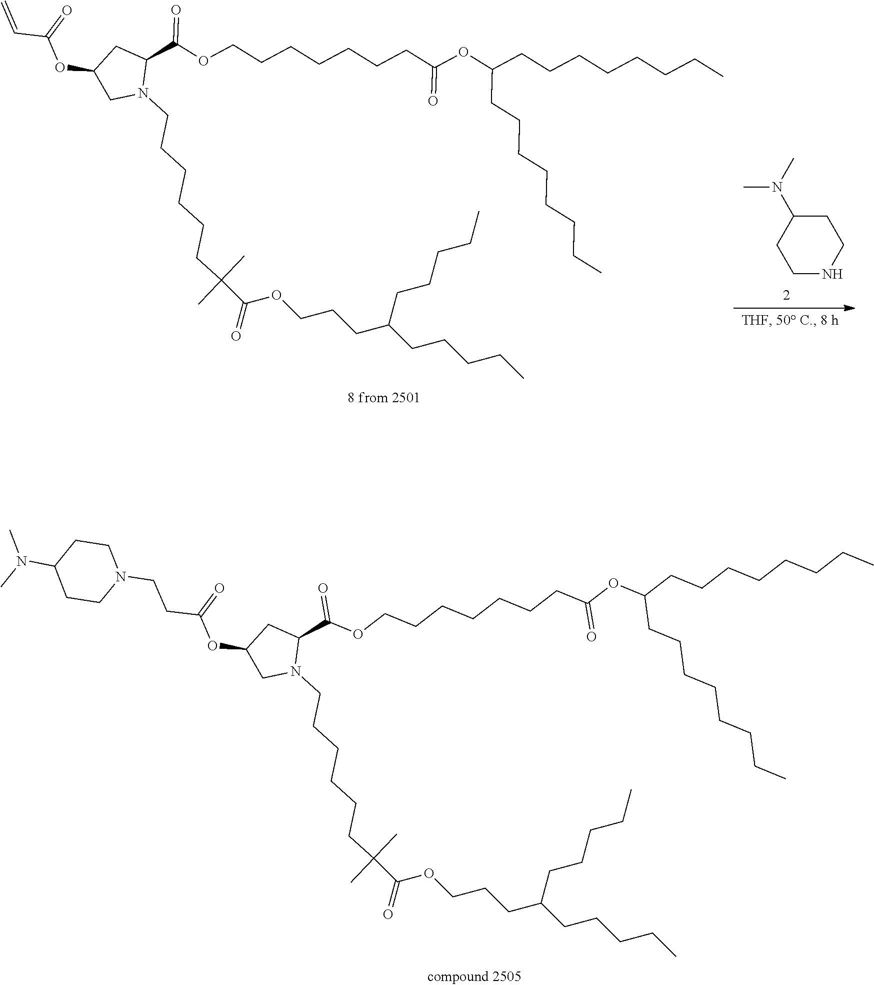

To a solution of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxylate (500 mg, 536.23 μmol, 1 eq) in THF (5 mL) was added N,N-dimethylpiperidin-4-amine (343.76 mg, 2.68 mmol, 5 eq). The mixture was stirred at 50° C. for 8 hr. The mixture was concentrated under reduced pressure to give a residue. The residue was purified by column chromatography (SiO₂, Petroleum ether/Ethyl acetate=10/1 to 0/1). The reaction mixture was diluted with PE 10 mL and extracted with ACN 40 mL (20 mL*2). The PE layers were concentrated under reduced pressure to give compound [8-(1-octylnonoxy)-8-oxo-octyl] (2S,4S)-4-[3-[4-(dimethylamino)-1-piperidyl]propanoyloxy]-1-[7,7-dimethyl-8-oxo-8-(4-pentylnonoxy)octyl]pyrrolidine-2-carboxylate (80 mg, 145.99 μmol, 18.99% yield, 95% purity) as yellow oil.

¹H NMR (400 MHz, CDCl₃), 5.14-5.22 (m, 1H), 4.83-4.86 (m, 1H), 3.99-4.12 (m, 4H), 2.93-3.24 (m, 4H), 2.24-2.73 (m, 18H), 1.87-2.02 (m, 5H), 1.55-1.72 (m, 12H), 1.43-1.54 (m, 6H), 1.17-1.38 (m, 52H), 1.13 (s, 6H), 0.85-0.89 (m, 12H). (M+H⁺): 1061.8.

LCMS-CAD: (M+H⁺): 1061.8 @ 9.741 min. LCMS-ELSD: (M+H⁺): 1060.9 @ 10.355 min.

### 9.30. Synthesis of 2508

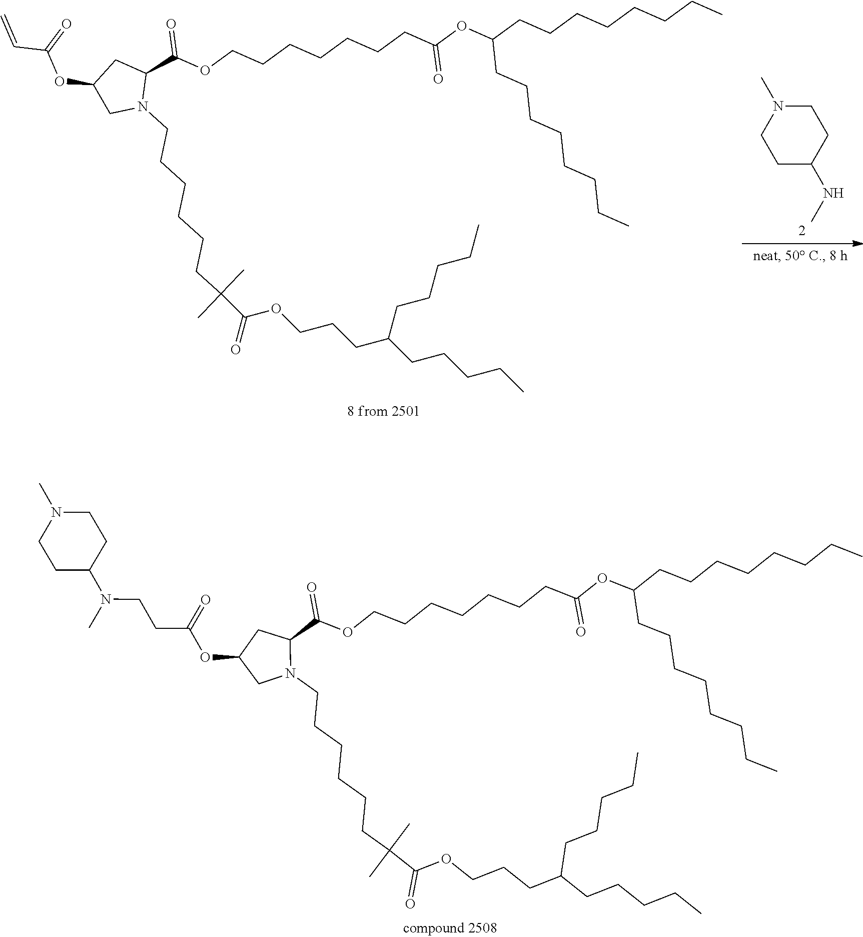

A mixture of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxylate (0.5 g, 536.23 μmol, 1 eq) in N, 1-dimethylpiperidin-4-amine (5 mL) was degassed and purged with N₂ for 3 times, and then the mixture was stirred at 50° C. for 8 hr under N₂ atmosphere. The crude reaction mixture was concentrated under reduced pressure to give a residue. The residue was purified by column chromatography (SiO₂, Petroleum ether/Ethyl acetate=10/1 to 0/1, added 5% NH₃·H₂O). The residue was purified by prep-HPLC (column: Xselect CSH C18 100*30 mm*5 μm; mobile phase: [H₂O (0.1% TFA)-ACN:THF-1:1]; gradient: 35%-65% B over 12.0 min). The mixture was diluted with brine 100 mL and extracted with EtOAc 60 mL (20 mL*3). The combined organic layers were dried over Na₂SO₄, filtered and concentrated under N₂ atmosphere to give a residue. The residue was diluted with hexane 20 ml, and washed with the mixture of ACN and TEA 60 mL (20 mL*3, 10:1). The hexane phase was concentrated under N₂ atmosphere to give a residue. The residue was diluted with hexane 20 mL and washed with ACN 40 mL (20 mL*2). The hexane phase was concentrated under N₂ atmosphere to give compound [8-(1-octylnonoxy)-8-oxo-octyl] (2S,4S)-1-[7,7-dimethyl-8-oxo-8-(4-pentylnonoxy)octyl]-4-[3-[methyl-(1-methyl-4-piperidyl)amino] propanoyloxy]pyrrolidine-2-carboxylate (0.06 g, 54.87 μmol, 29.10% yield, 97% purity) as yellow oil.

¹H NMR (400 MHz, CDCl₃), 5.14-5.21 (m, 1H), 4.85-4.88 (m, 1H), 4.01-4.14 (m, 4H), 3.07-3.26 (m, 2H), 2.92-2.95 (m, 2H), 2.24-2.79 (m, 18H), 1.71-2.06 (m, 6H), 1.45-1.70 (m, 13H), 1.20-1.38 (m, 56H), 1.15 (s, 6H), 0.86-0.90 (m, 12H), (M+H⁺): 1060.8.

LCMS-CAD: (M+H⁺): 1060.8 @ 11.759 min.

LCMS-ELSD: (M+H⁺): 1060.8 @ 12.220 min.

### 9.31. Synthesis of 2510

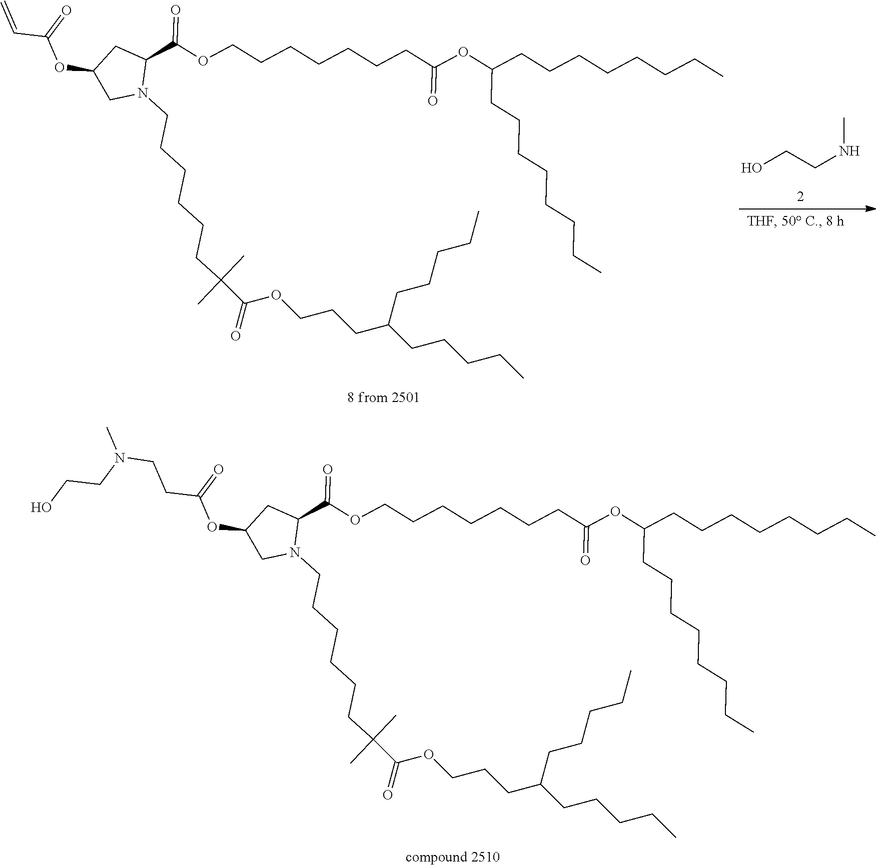

To a solution of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxy late (500 mg, 536.23 μmol, 1 eq) in THF (5 mL) was added 2-(methylamino) ethanol (201.38 mg, 2.68 mmol, 215.38 μL, 5 eq). The mixture was stirred at 50° C. for 8 hr. The mixture was concentrated under reduced pressure to give a residue. The residue was purified by column chromatography (SiO₂, Petroleum ether/Ethyl acetate=10/1 to 0/1). The residue was purified by prep-HPLC (column: Xselect CSH C18 100*30 mm*5 μm; mobile phase: [H₂O (0.04% HCl)-THF:ACN=1:3]; gradient: 35%-75% B over 12.0 min). Then worked with NaHCO₃ aqueous 10 mL and extracted with EtOAc 15 mL (5 mL*3). The combined organic layers were dried over Na₂SO₄, filtered and concentrated under reduced pressure to give compound [8-(1-octylnonoxy)-8-oxo-octyl] (2S,4S)-1-[7,7-dimethyl-8-oxo-8-(4-pentylnonoxy)octyl]-4-[3-[2-hydroxyethyl(methyl)amino]propanoyloxy]pyrrolidine-2-carboxylate (0.08 g, 76.22 μmol, 66.32% yield, 96% purity) as yellow oil.

¹H NMR (400 MHz, CDCl₃), 5.18-5.26 (m, 1H), 4.85-4.88 (m, 1H), 4.01-4.14 (m, 4H), 3.63-3.72 (m, 2H), 3.27 (d, J=11.2 Hz, 1H), 3.10 (t, J=8.4 Hz, 1H), 2.55-2.86 (m, 9H), 2.26-2.38 (m, 6H), 1.98-2.07 (m, 1H), 1.56-1.66 (m, 6H), 1.45-1.55 (m, 8H), 1.22-1.38 (m, 55H), 1.15 (s, 6H), 0.86-0.90 (m, 12H). (M+H⁺): 1007.7. LCMS-CAD: (M+H⁺): 1007.7 @ 11.476 min.

LCMS-ELSD: (M+H⁺): 1007.9 @ 11.839 min.

### 9.32. Synthesis of 2485

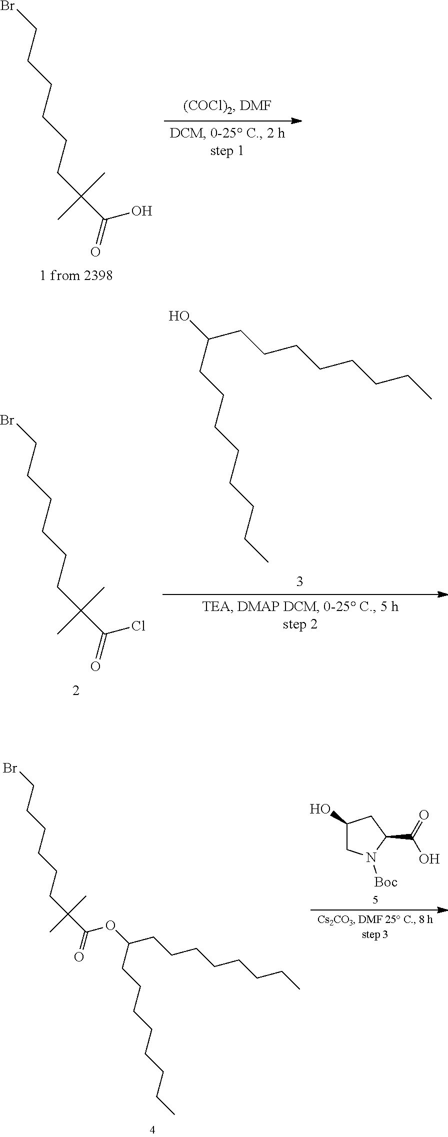 [SMILES: CC(C)(CCCCCCBr)C(=O)O ; CC(C)(CCCCCCBr)C(=O)Cl ; CCCCCCCCC(O)CCCCCCCC ; CCCCCCCCC(CCCCCCCC)OC(=O)C(C)(C)CCCCCCBr ; CC(C)(C)OC(=O)N1C[C@@H](O)C[C@H]1C(=O)O]

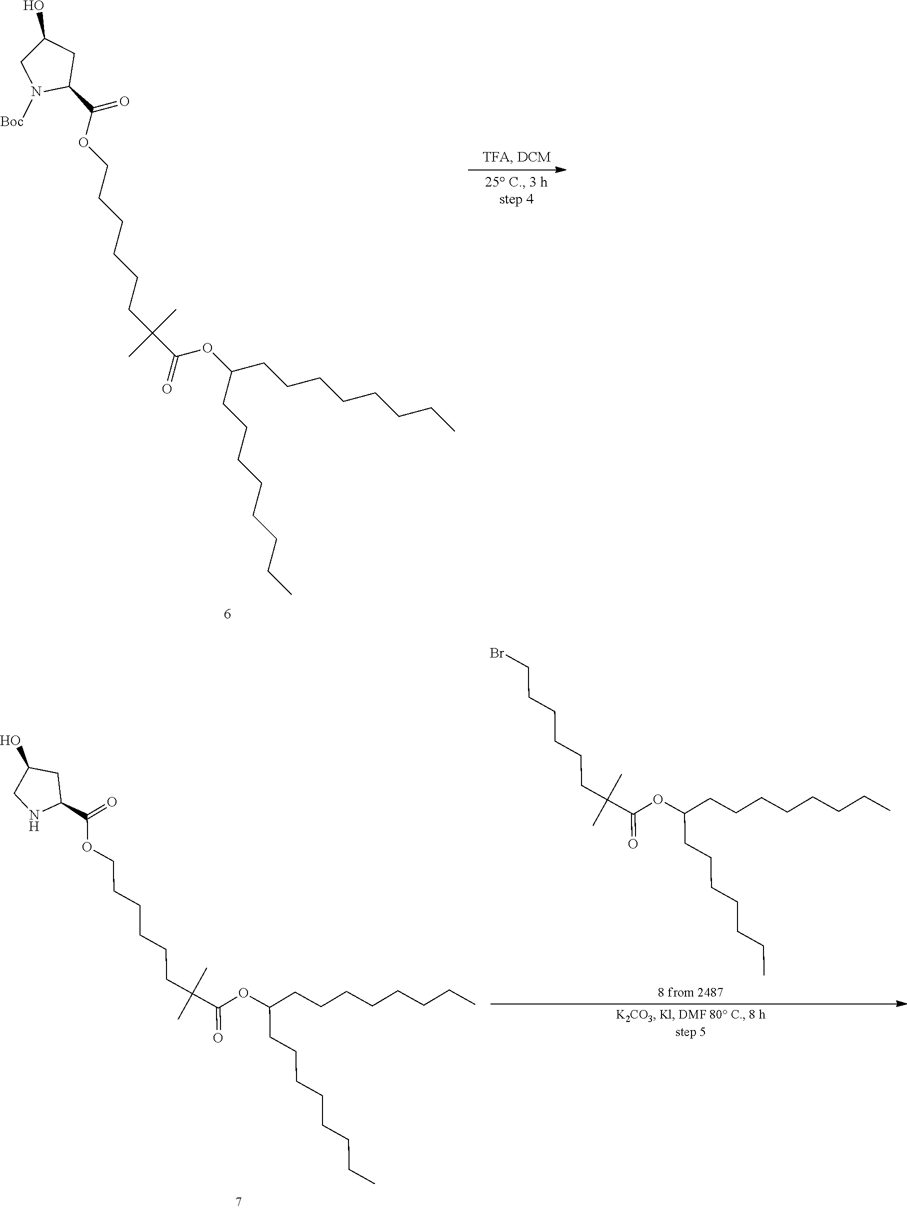 [SMILES: CCCCCCCCC(CCCCCCCC)OC(=O)C(C)(C)CCCCCCOC(=O)[C@@H]1C[C@H](O)CN1C(=O)OC(C)(C)C ; CCCCCCCCC(CCCCCCCC)OC(=O)C(C)(C)CCCCCCOC(=O)[C@@H]1C[C@H](O)CN1 ; CCCCCCCCC(CCCCCCC)OC(=O)C(C)(C)CCCCCCBr]

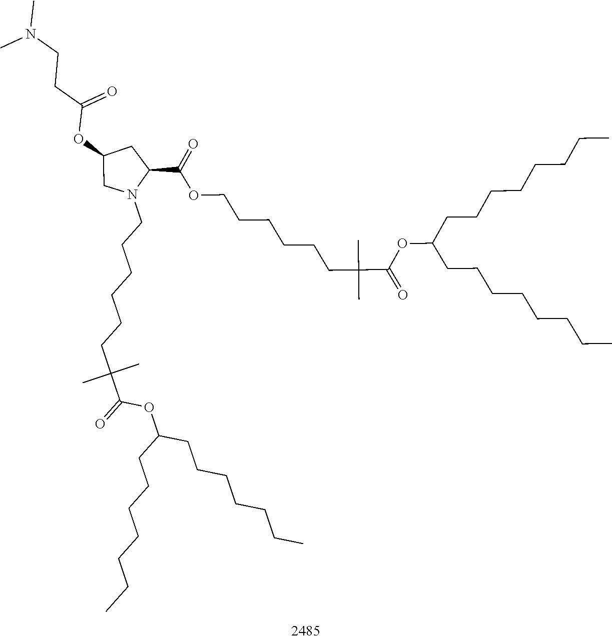 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OC(CCCCCCC)CCCCCCC]

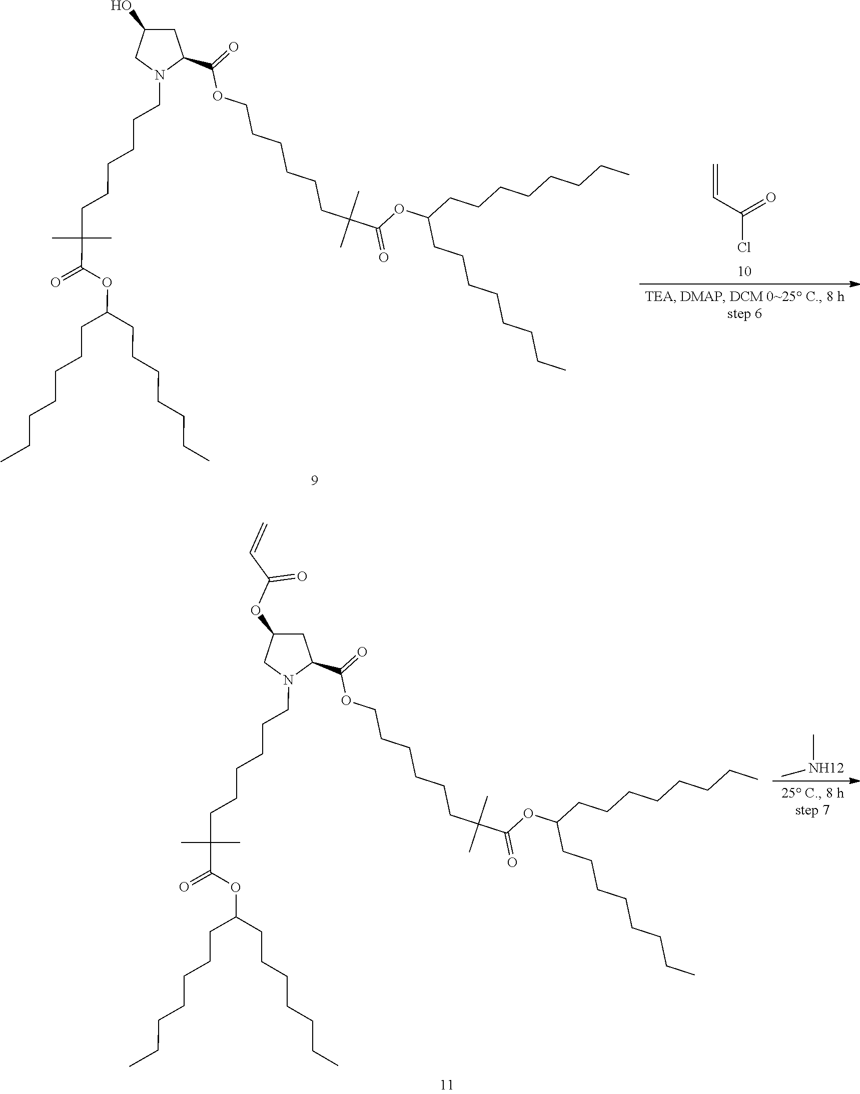

### Step 1:

A mixture of 8-bromo-2,2-dimethyl-octanoic acid (5 g, 19.91 mmol, 1 eq) in DCM (50 mL) was added (COCl)₂ (12.63 g, 99.54 mmol, 8.71 mL, 5 eq), DMF (29.10 mg, 398.15 μmol, 30.63 μL, 0.02 eq) at 0° C. The mixture was stirred at 25° C. for 2 hr under N₂ atmosphere. The reaction mixture was concentrated under reduced pressure to give compound 8-bromo-2,2-dimethyl-octanoyl chloride (5 g, crude) as yellow oil.

### Step 2:

To a solution of heptadecan-9-ol (4.32 g, 16.86 mmol, 1 eq), TEA (8.53 g, 84.30 mmol, 11.73 mL, 5 eq). DMAP (411.95 mg, 3.37 mmol, 0.2 eq) in DCM (80 mL) was added 8-bromo-2,2-dimethyl-octanoyl chloride (5 g, 18.55 mmol, 1.1 eq) at 0° C. The mixture was stirred at 25° C. for 5 hr. The combined organic phase was diluted with EtOAc 60 mL and washed with water 90 mL (30 mL*3) and brine 60 mL (30 mL*2), dried with anhydrous Na₂SO₄, filtered and concentrated under reduced pressure to give a residue. The residue was purified by column chromatography (SiO₂, Petroleum ether/Ethyl acetate=1/0)) to give compound 1-octylnonyl 8-bromo-2,2-dimethyl-octanoate (5 g, crude) as colorless oil.

### Step 3:

A mixture of 1-octylnonyl 8-bromo-2,2-dimethyl-octanoate (6 g, 12.25 mmol, 1 eq), (2S,4S)-1-tert-butoxy carbonyl-4-hydroxy-pyrrolidine-2-carboxylic acid (3.40 g, 14.71 mmol, 1.2 eq), Cs₂CO₃ (8.78 g, 26.96 mmol, 2.2 eq) in DMF (50 mL) was degassed and purged with N₂ for 3 times, and then the mixture was stirred at 25° C. for 8 hr under N₂ atmosphere. The mixture is filtered through celite and the solvent was removed under reduced pressure to give a residue. The reaction mixture was diluted with H₂O 60 mL and extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=10/1 to 8/1, added 3% NH₃·H₂O) to give compound O1-tert-butyl O2-[7,7-dimethyl-8-(1-octylnonoxy)-8-oxo-octyl] (2S,4S)-4-hydroxypyrrolidine-1,2-dicarboxylate (3.2 g, 5.00 mmol, 40.80% yield) as yellow oil.

¹H NMR (400 MHz, CDCl₃), 4.76-4.85 (m, 1H), 4.08-4.37 (m, 4H), 3.51-3.75 (m, 2H), 2.27-2.42 (m, 1H), 2.06-2.12 (m, 1H), 1.62-1.70 (m, 3H), 1.44-1.48 (m, 12H), 1.21-1.39 (m, 32H), 1.15 (s, 6H), 0.85-0.90 (m, 6H).

### Step 4:

To a solution of O1-tert-butyl O2-[7,7-dimethyl-8-(1-octylnonoxy)-8-oxo-octyl] (2S,4S)-4-hydroxypyrrolidine-1,2-dicarboxylate (3.2 g, 5.00 mmol, 1 eq) in DCM (15 mL) was added TFA (7.68 g, 67.31 mmol, 5 mL, 13.46 eq). The mixture was stirred at 25° C. for 3 hr. The reaction mixture was adjusted pH to 7 with saturated NaHCO₃ aqueous and extracted with EtOAc 30 mL (10 mL*3), dried over Na₂SO₄, filtered and concentrated under reduced pressure to give a residue. The residue was purified by column chromatography (SiO₂, Petroleum ether/Ethyl acetate=1/0 to 1/1, added 3% NH₃·H₂O) to give compound [7,7-dimethyl-8-(1-octylnonoxy)-8-oxo-octyl] (2S,4S)-4-hydroxypyrrolidine-2-carboxylate (2.6 g, 4.82 mmol, 96.32% yield) as yellow oil.

### Step 5:

To a solution of 1-heptyloctyl 8-bromo-2,2-dimethyl-octanoate (2.67 g, 5.78 mmol, 1.2 eq). [7,7-dimethyl-8-(1-octylnonoxy)-8-oxo-octyl] (2S,4S)-4-hydroxypyrrolidine-2-carboxylate (2.6 g, 4.82 mmol, 1 eq) in DMF (10 mL) was added K₂CO₃ (2.66 g, 19.27 mmol, 4 eq), KI (799.52 mg, 4.82 mmol, 1 eq). The mixture was stirred at 80° C. for 8 hr. The reaction mixture was diluted with H₂O 20 mL and extracted with EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate/NH₃·H₂O=10/1/1 to 1/1/0.5) to give compound [7,7-dimethyl-8-(1-octylnonoxy)-8-oxo-octyl] (2S,4S)-1-[8-(1-heptyloctoxy)-7,7-dimethyl-8-oxo-octyl]-4-hydroxy-pyrrolidine-2-carboxylate (3 g, 3.26 mmol, 67.67% yield) as yellow oil.

### Step 6:

To a solution of [7,7-dimethyl-8-(1-octylnonoxy)-8-oxo-octyl] (2S,4S)-1-[8-(1-heptyloctoxy)-7,7-dimethyl-8-oxo-octyl]-4-hydroxy-pyrrolidine-2-carboxylate (3 g, 3.26 mmol, 1 eq), DMAP (39.82 mg, 325.92 μmol, 0.1 eq), TEA (2.97 g, 29.33 mmol, 4.08 mL, 9 eq) in DCM (10 mL) was added prop-2-enoyl chloride (1.47 g, 16.30 mmol, 1.33 mL, 5 eq) at 0° C. The mixture was stirred at 25° C. for 8 hr. The combined organic phase was diluted with EtOAc 30 mL and washed with water 60 mL (20 mL*3) and brine 40 mL (20 mL*2), dried with anhydrous Na₂SO₄, filtered and concentrated under reduced pressure to give a residue. The residue was purified by column chromatography (SiO₂, Petroleum ether/Ethyl acetate=20/1 to 5/1, added 3% NH₃·H₂O) to give compound [7,7-dimethyl-8-(1-octylnonoxy)-8-oxo-octyl] (2S,4S)-1-[8-(1-heptyloctoxy)-7,7-dimethyl-8-oxo-octyl]-4-prop-2-enoyloxy-pyrrolidine-2-carboxylate (0.8 g, 820.92 μmol, 25.19% yield) as yellow oil.

¹H NMR (400 MHz, CDCl₃), 6.42 (d, J=17.2 Hz, 1H), 6.09-6.19 (m, 1H), 5.82 (d, J=10.4 Hz, 1H), 5.24-5.30 (m, 1H), 4.79-4.87 (m, 2H), 4.08-4.15 (m, 2H), 3.13-3.30 (m, 2H), 2.58-2.82 (m, 3H), 2.06-2.35 (m, 2H), 1.45-1.56 (m, 12H), 1.21-1.38 (m, 60H), 1.15 (d, J=2.4 Hz, 12H), 0.88 (t, J=6.4 Hz, 12H).

### Step 7:

A mixture of [7,7-dimethyl-8-(1-octylnonoxy)-8-oxo-octyl] (2S,4S)-1-[8-(1-heptyloctoxy)-7,7-dimethyl-8-oxo-octyl]-4-prop-2-enoyloxy-pyrrolidine-2-carboxylate (0.8 g, 820.92 μmol, 1 eq) in N-methylmethanamine (2 M, 410.46 μL, 1 eq) was degassed and purged with N₂ for 3 times, and then the mixture was stirred at 25° C. for 8 hr under N₂ atmosphere. The reaction mixture was concentrated under reduced pressure to get a residue. The residue was purified by column chromatography (SiO₂, Petroleum ether/Ethyl acetate=10/1 to 3/1, added 3% NH₃·THF) to give compound [7,7-dimethyl-8-(1-octylnonoxy)-8-oxo-octyl] (2S,4S)-4-[3-(dimethylamino)propanoyloxy]-1-[8-(1-heptyloctoxy)-7,7-dimethyl-8-oxo-octyl]pyrrolidine-2-carboxylate (0.161 g, 168.20 μmol, 21.44% yield, 98% purity) was obtained as yellow oil.

¹H NMR (400 MHz, CDCl₃), 5.18-5.24 (m, 1H), 4.81-4.85 (m, 2H), 4.09-4.14 (m, 2H), 3.07-3.26 (m, 2H), 2.52-2.77 (m, 7H), 2.21-2.35 (m, 7H), 1.98-2.06 (m, 1H), 1.62-1.68 (m, 2H), 1.45-1.57 (m, 12H), 1.17-1.37 (m, 58H), 1.15 (d, J=2.8 Hz, 12H), 0.88 (t, J=6.4 Hz, 12H), (M+H⁺): 1019.7. LCMS: (M+H⁺): 1019.7 @ 13.968 min. LCMS: (M+H⁺): 1019.8 @ 14.450 min.

### 9.33. Synthesis of 2499

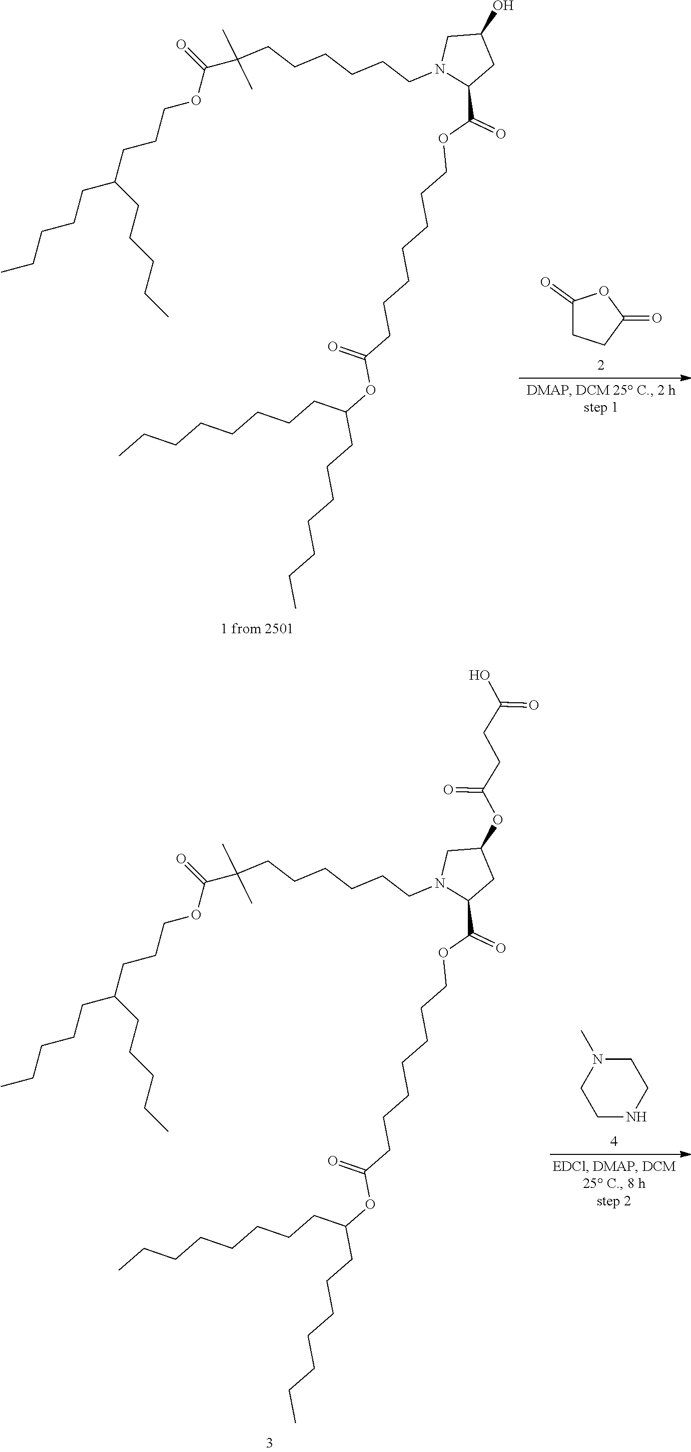

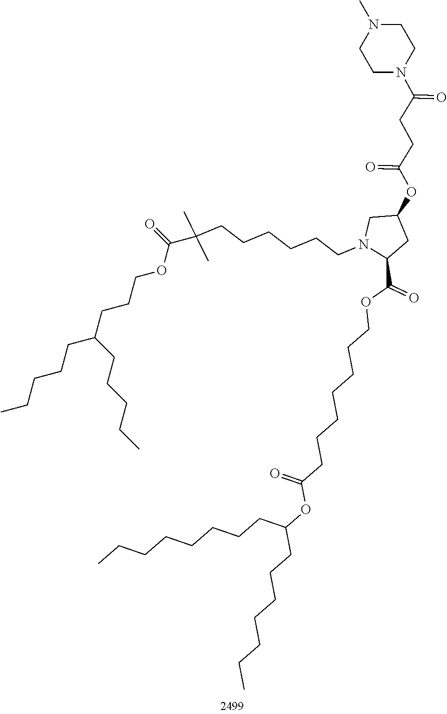 [SMILES: CCCCCCCCC(CCCCCCC)OC(=O)CCCCCCCOC(=O)[C@@H]1C[C@H](OC(=O)CCC(=O)N2CCN(C)CC2)CN1CCCCCCC(C)(C)C(=O)OCCCC(CCCCC)CCCCC]

### Step 1:

To a solution of [8-(1-octylnonoxy)-8-oxo-octyl] (2S,4S)-1-[7,7-dimethyl-8-oxo-8-(4-pentylnonoxy)octyl]-4-hydroxy-pyrrolidine-2-carboxy late (0.460 g, 523.68 μmol, 1 eq) in DCM (5 mL) was added DMAP (127.95 mg, 1.05 mmol, 2 eq) and tetrahydrofuran-2,5-dione (157.22 mg, 1.57 mmol, 3 eq). The mixture was stirred at 25° C. for 2 hr. The reaction mixture was concentrated under reduced pressure to remove solvent. The residue was purified by prep-TLC (Ethyl acetate:Methanol=5:1) to give compound 4-[(3S,5S)-1-[7,7-dimethyl-8-oxo-8-(4-pentylnonoxy)octyl]-5-[8-(1-octylnonoxy)-8-oxo-octoxy]carbonylpyrrolidin-3-yl]oxy-4-oxo-butanoic acid (0.230 g, 235.06 μmol, 44.92% yield) as a brown oil.

### Step 2:

To a solution of 4-[(3S,5S)-1-[7,7-dimethyl-8-oxo-8-(4-pentylnonoxy)octyl]-5-[8-(1-octylnonoxy)-8-oxooctoxy]carbonyl-pyrrolidin-3-yl]oxy-4-oxo-butanoic acid (0.23 g, 235.06 μmol, 1 eq) in DCM (5 mL) was added 1-methylpiperazine (28.25 mg, 282.07 μmol, 31.29 μL, 1.2 eq), EDCI (67.59 mg, 352.59 μmol, 1.5 eq) and DMAP (14.36 mg, 117.53 μmol, 0.5 eq). The mixture was stirred at 25° C. for 8 hr. The reaction mixture was concentrated under reduced pressure to give a residue. The residue was purified by prep-TLC (Ethyl acetate:Methanol-3:1). The residue was purified by prep-HPLC ([H₂O (0.04% HCl)-ACN:THF=1:1]). The mixture extracted with EtOAc 150 mL (50 mL*3). The combined organic layers were dried over Na₂SO₄, filtered and concentrated under N₂. Then the mixture was adjusted pH to 8 with ACN/TEA=10/1 50 mL, extracted with hexane 60 mL (20 mL*3). The hexane layers were concentrated under N₂ to give compound [8-(1-octylnonoxy)-8-oxo-octyl] (2S,4S)-1-[7,7-dimethyl-8-oxo-8-(4-pentylnonoxy)octyl]-4-[4-(4-methylpiperazin-1-yl)-4-oxo-butanoyl]oxypyrrolidine-2-carboxy late (67 mg, 63.2 μmol, 26.9% yield) as a colorless oil.

¹H NMR (400 MHz, CDCl₃), 5.17-5.25 (m, 1H), 4.85-4.88 (m, 1H), 4.01-4.14 (m, 4H), 3.53-3.73 (m, 4H), 3.06-3.28 (m, 2H), 2.23-2.80 (m, 17H), 2.00-2.07 (m, 1H), 1.57-1.65 (m, 10H), 1.43-1.56 (m, 6H), 1.18-1.40 (m, 53H), 1.15 (s, 6H), 0.86-0.91 (m, 12H), (M+H⁺): 1060.7.

LCMS: (M+H⁺): 1060.7 @ 13.178 min. LCMS: (M+H⁺): 1060.8 @ 9.564 min.

### 9.34. Synthesis of 2507

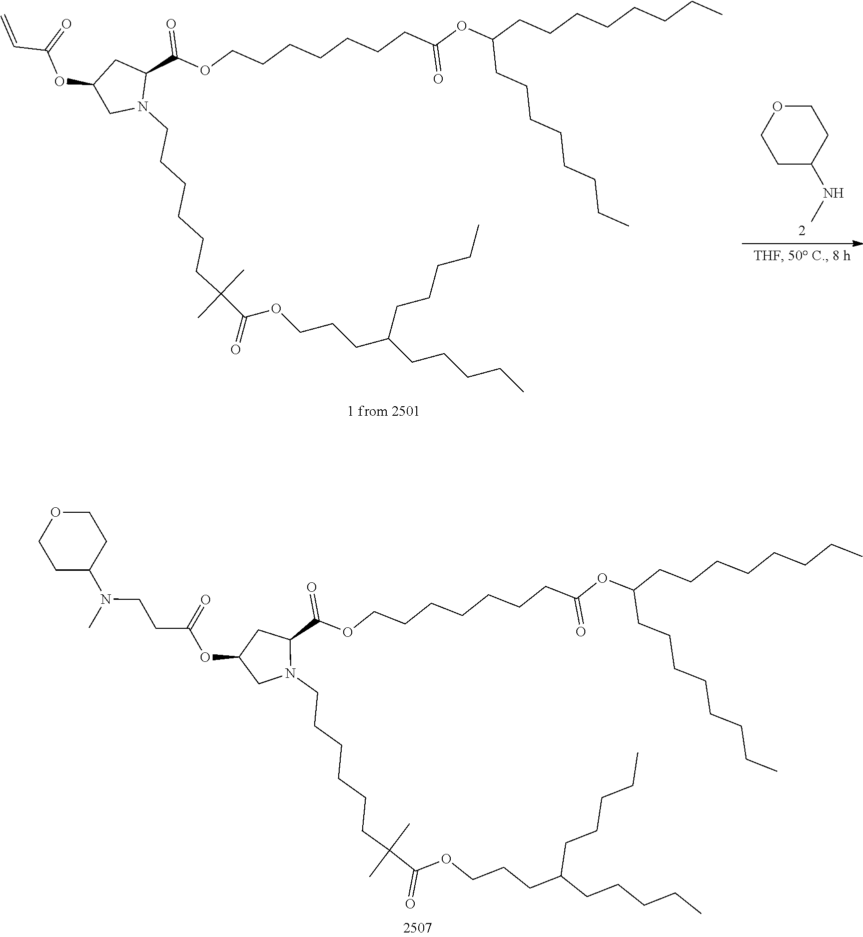

A mixture of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxylate (0.5 g, 536.23 μmol, 1 eq) and N-methyltetrahydropyran-4-amine (185.28 mg, 1.61 mmol, 3 eq) in THF (5 mL) was degassed and purged with N₂ for 3 times, and then the mixture was stirred at 50° C. for 8 hr under N₂ atmosphere. The crude reaction mixture was concentrated under reduced pressure to give a residue. The residue was purified by column chromatography (SiO₂, Petroleum ether/Ethyl acetate=10/1 to 0/1, added 5% NH₃·H₂O). The residue was purified by prep-HPLC (column: Xselect CSH C18 100*30 mm*5 um; mobile phase: [H₂O (0.1% TFA)-ACN:THF=1:1]; gradient: 34%-74% B over 12.0 min). The mixture extracted with EtOAc 150 mL (50 mL*3). The combined organic layers were dried over Na₂SO₄, filtered and concentrated under N₂. Then the mixture was adjusted pH to 8 with ACN/TEA=10/1 50 mL, extracted with hexane 60 mL (20 mL*3). The hexane layers were concentrated under N₂ to give compound [8-(1-octylnonoxy)-8-oxo-octyl] (2S,4S)-1-[7,7-dimethyl-8-oxo-8-(4-pentylnonoxy)octyl]-4-[4-(4-methylpiperazin-1-yl)-4-oxo-butanoyl]oxy-pyrrolidine-2-carboxylate (124 mg, 118 μmol, 22.10% yield, 99% purity) as yellow oil.

¹H NMR (400 MHz, CDCl₃), 5.18-5.24 (m, 1H), 4.85-4.88 (m, 1H), 4.12 (t, J=6.8 Hz, 2H), 4.03 (t, J=6.4 Hz, 4H), 3.37 (t, J=11.6 Hz, 2H), 3.25 (d, J=11.2 Hz, 1H), 3.10 (t, J=8.0 Hz, 1H), 2.70-2.92 (m, 3H), 2.48-2.65 (m, 4H), 2.23-2.35 (m, 5H), 1.98-2.07 (m, 1H), 1.56-1.75 (m, 14H), 1.45-1.55 (m, 8H), 1.19-1.40 (m, 53H), 1.15 (s, 6H), 0.86-0.91 (m, 12H), (M+H⁺): 1047.8. LCMS: (M+H⁺): 1047.8, 10.093 min. LCMS: (M+H⁺): 1047.8 @ 10.128 min.

### 9.35. Synthesis of 2511

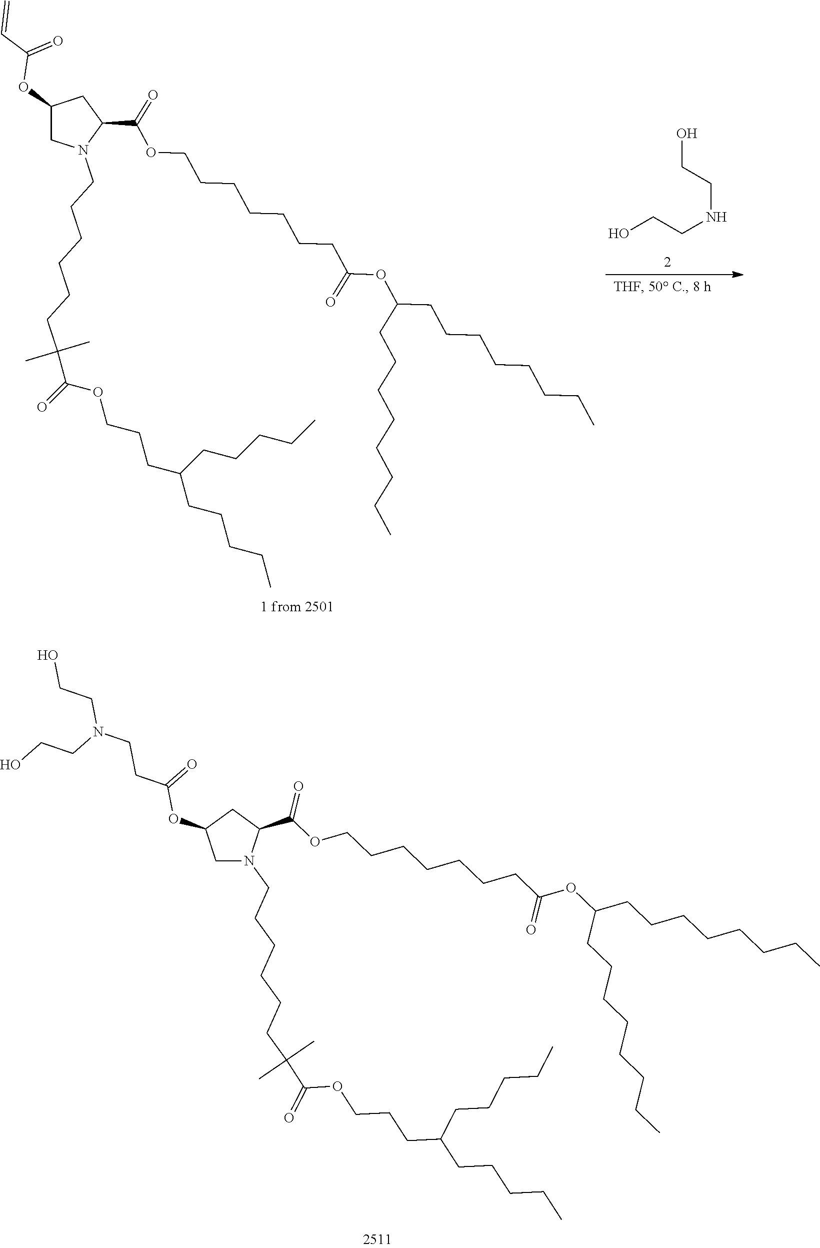 [SMILES: C=CC(=O)O[C@H]1C[C@@H](C(=O)OCCCCCCCC(=O)OC(CCCCCCCC)CCCCCCCC)N(CCCCCCC(C)(C)C(=O)OCCCC(CCCCC)CCCCC)C1 ; OCCNCCO ; CCCCCCCCC(CCCCCCCC)OC(=O)CCCCCCCOC(=O)[C@@H]1C[C@H](OC(=O)CCN(CCO)CCO)CN1CCCCCCC(C)(C)C(=O)OCCCC(CCCCC)CCCCC]

To a solution of [8-(1-octylnonoxy)-8-oxo-octyl] (2S,4S)-1-[7,7-dimethyl-8-oxo-8-(4-pentylnonoxy)octyl]-4-prop-2-enoyloxy-pyrrolidine-2-carboxylate (900 mg, 965.21 μmol, 1 eq) in THF (10 mL) was added 2-(2-hydroxyethylamino) ethanol (101.48 mg, 965.21 μmol, 93.27 μL, 1 eq). The mixture was stirred at 50° C. for 8 hr. The reaction mixture was quenched by addition H₂O 10 mL at 0° C., and then extracted with EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate=10/1 to 0/1). The residue was purified by prep-HPLC (column: Xselect CSH C18 100*30 mm*5 um; mobile phase: [H₂O (0.1% TFA)-ACN:THF=1:1]; gradient: 40%-80% B over 12.0 min). The mixture extracted with EtOAc 90 mL (30 mL*3). The combined organic layers were dried over Na₂SO₄, filtered and concentrated under N₂. Then the mixture was adjusted pH to 8 with ACN/TEA=10/1 20 mL, extracted with hexane 60 mL (20 mL*3). The mixture was concentrated under N₂. Then diluted with hexane 10 mL and extracted with ACN/MeOH=10/1 20 mL (10 mL*2).

The hexane layers were concentrated under N₂ to give compound [8-(1-octylnonoxy)-8-oxo-octyl] (2S,4S)-4-[3-[bis(2-hydroxyethyl)amino]propanoyloxy]-1-[7,7-dimethyl-8-oxo-8-(4pentylnonoxy)octyl]pyrrolidine-2-carboxylate (31 mg, 28.68 μmol, 59.52% yield, 99% purity) as a yellow oil.

¹H NMR (400 MHz, CDCl₃), 5.14-5.21 (m, 1H), 4.85-4.88 (m, 1H), 4.01-4.15 (m, 4H), 3.62-3.76 (m, 4H), 3.31 (d, J=11.2 Hz, 1H), 3.10 (t, J=8.4 Hz, 1H), 2.53-3.03 (m, 11H), 2.28 (t, J=7.6 Hz, 3H), 2.04-2.12 (m, 1H), 1.45-1.62 (m, 13H), 1.17-1.41 (m, 56H), 1.15 (s, 6H), 0.87-0.91 (m, 12H), (M+H⁺): 1037.7. LCMS: (M+H⁺): 1037.7 @ 13.331 min.

LCMS: (M+H⁺): 1037.9 @ 9.744 min.

### 9.P1. Synthesis of Compounds 2535, 2536, 2537, 2552, 2553, 2554, 2555, 2556, 2557, 2559, 2560, 2561, 2563, 2564, 2565, 2566, 2567, 2568, 2569, 2570, 2571, 2573, 2574, 2575, A1-A36 and A45-A106

Starting from commercially available reagents, 2535, 2536, 2537, 2552, 2553, 2554, 2555, 2556, 2557, 2559, 2560, 2561, 2563, 2564, 2565, 2566, 2567, 2568, 2569, 2570, 2571, 2573, 2574, 2575, A1-A36, and A45-A106, are prepared following the procedure for the synthesis of compound 2392, with minor modifications.

### 9.P2. Synthesis of Compound A37

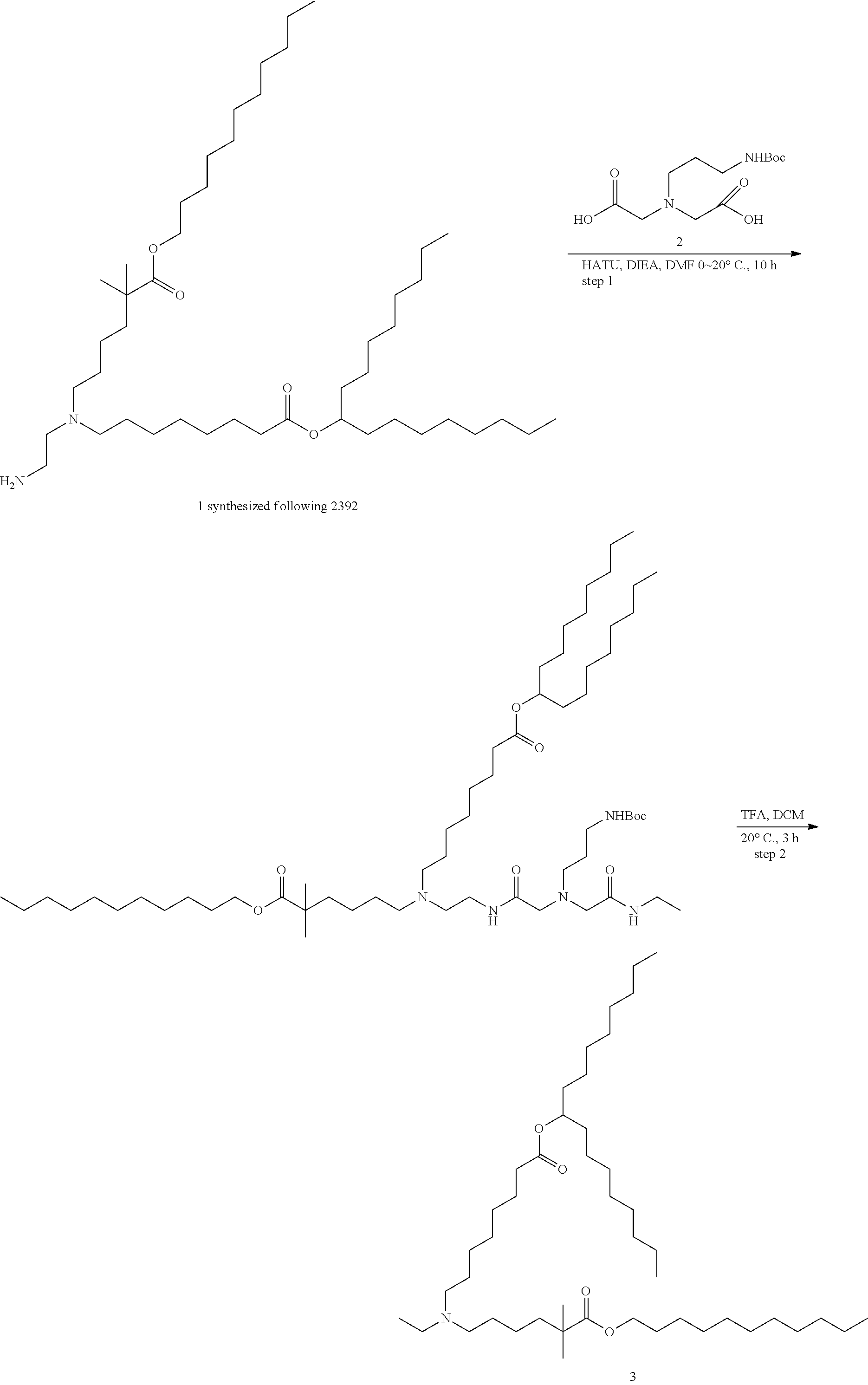

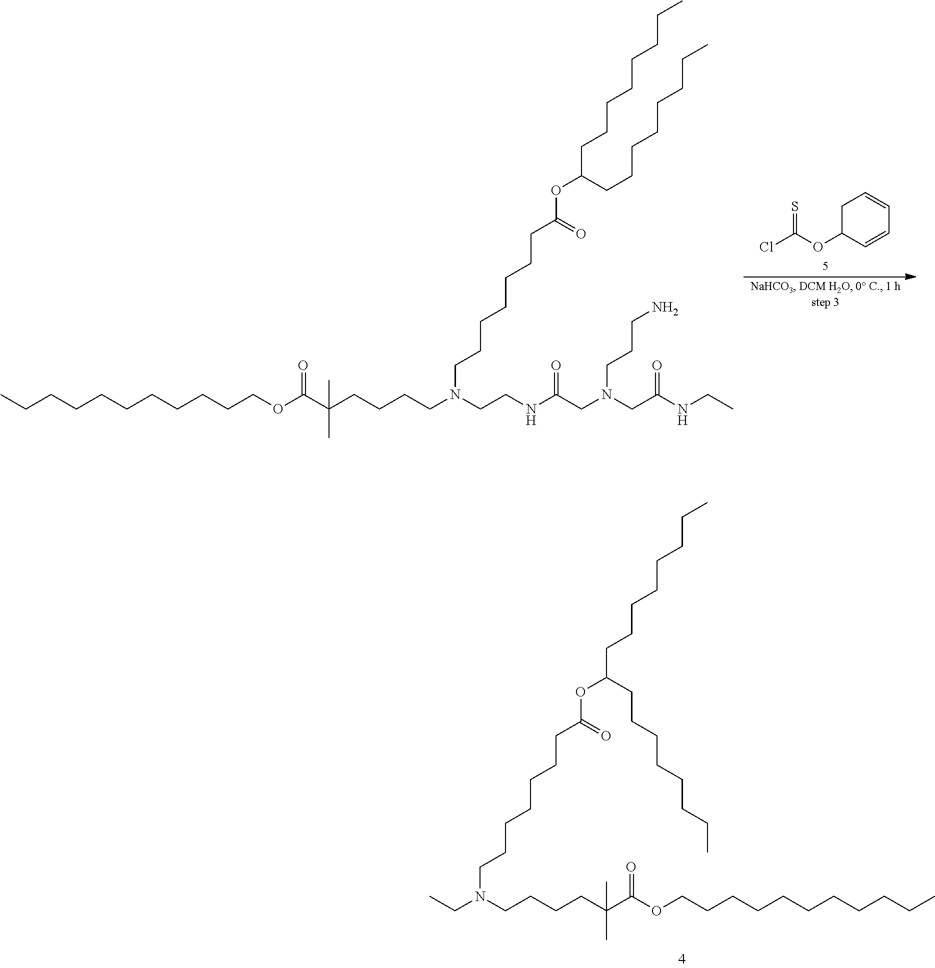

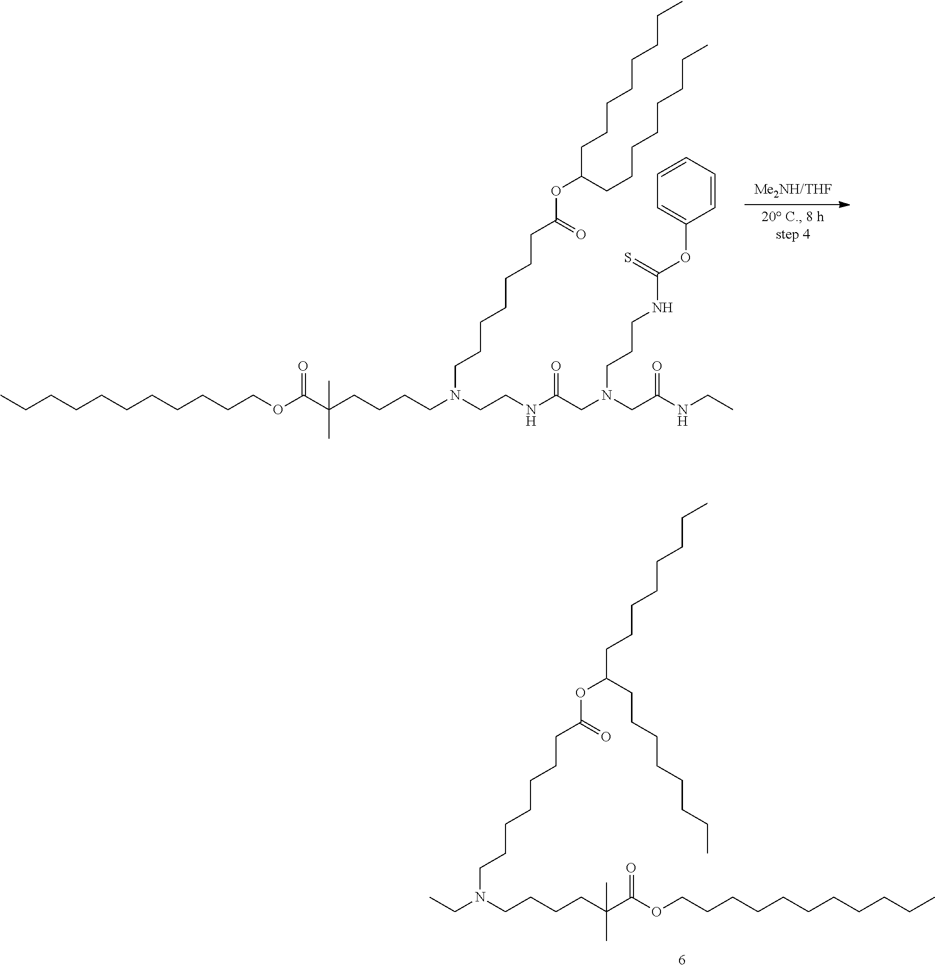

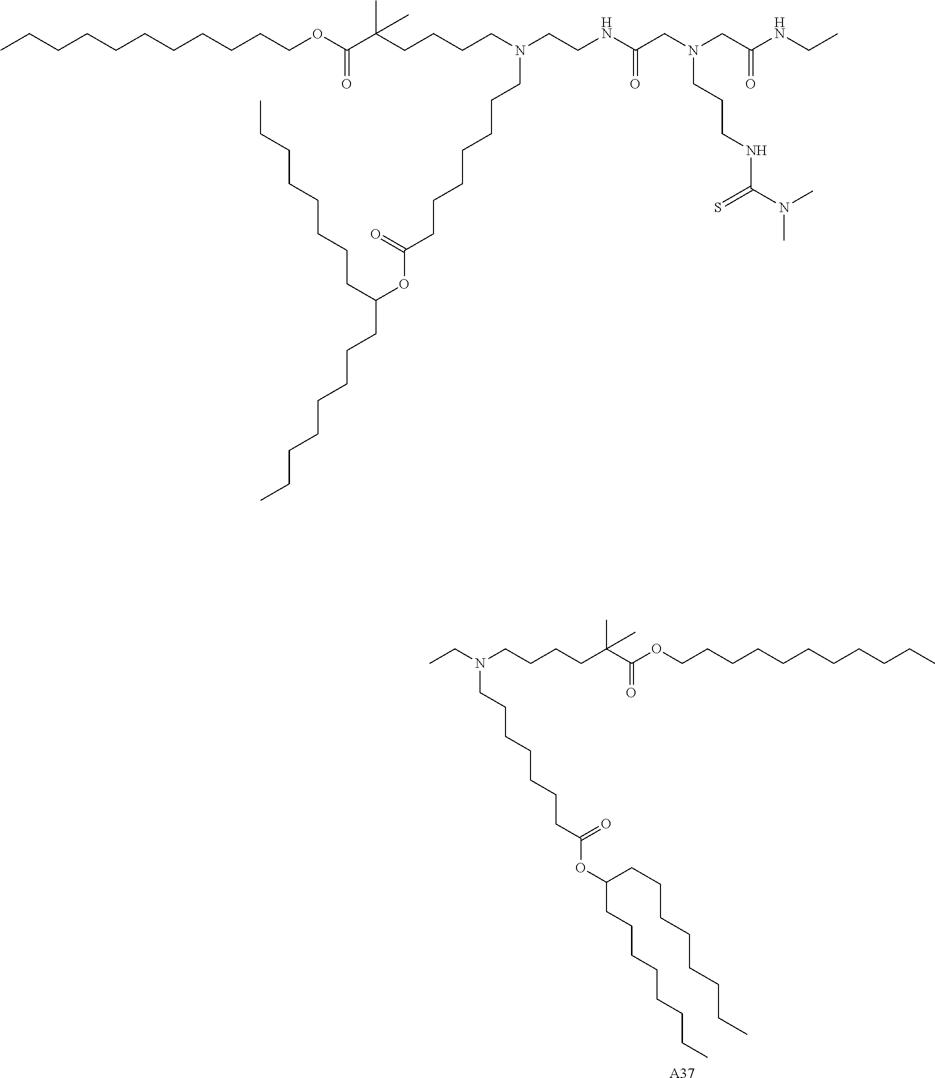

### Step 1:

To a solution of 2 (409.36 mg, 1.41 mmol, 1 eq) in DMF (40 mL) is added HATU (1.34 g, 3.53 mmol, 2.5 eq) and DIEA (455.61 mg, 3.53 mmol, 614.03 μL, 2.5 eq) in sequence at 0° C. Then the mixture is stirred at 0° C. for 2 hr. Then 1 (2 g, 2.82 mmol, 2 eq) is added at 0° C. Then the mixture is stirred at 20° C. for 8 hr. The reaction mixture is diluted with addition H₂O 100 mL, and then extracted with EtOAc 90 mL (30 mL*3). The combined organic layers are washed with sat, brine 80 mL (40 mL*2), dried over Na₂SO₄, filtered and concentrated under reduced pressure to give a residue. The residue is purified by column chromatography (SiO₂, Petroleum ether/Ethyl acetate=1/0 to 0/1) to give 3 (2 g, crude) as yellow oil.

### Step 2:

To a solution of 3 (2 g, 1.20 mmol, 1 eq) in DCM (12 mL) is added TFA (9.21 g, 80.78 mmol, 6 mL, 67.55 eq). Then the mixture is stirred at 20° C. for 3 hr. The reaction mixture is concentrated under reduced pressure to remove solvent. The reaction mixture is adjusted to pH 8 with sat. NaHCO₃, and then extracted EtOAc 45 mL (15 mL*3). The combined organic layers are dried over Na₂SO₄, filtered and concentrated under reduced pressure to give a residue. The residue is purified by column chromatography (SiO₂, Petroleum ether/Ethyl acetate=5/1 to 0/1) to give compound 4 (500 mg, 308.42 μmol, 25.79% yield, 97% purity) as a colorless oil.

### Step 3:

To a solution of 4 (400 mg, 254.37 μmol, 1 eq) in DCM (5 mL) is added NaHCO₃ (23.51 mg, 279.81 μmol, 10.89 μL, 1.1 eq) in H₂O (2 mL) and O-phenyl chloromethanethioate (52.69 mg, 305.24 μmol, 42.22 μL, 1.2 eq) in sequence. Then the mixture is stirred at 0° C. for 1 hr. The reaction mixture is concentrated under reduced pressure to remove solvent. The residue is purified by column chromatography (SiO₂, Petroleum ether/Ethyl acetate=5/1 to 1/1) to give compound 5 (300 mg, 175.57 μmol, 42.86% yield) as a colorless oil.

### Step 4:

A solution of 5 (150 mg, 87.79 μmol, 1 eq) in N-methylmethanamine (2 M/THF, 5 mL, 113.91 eq) is stirred at 20° C. for 8 hr. The reaction mixture is concentrated under reduced pressure to remove solvent. The residue is purified by prep-TLC (SiO₂, Petroleum ether/Ethyl acetate=3/1). The reaction mixture is diluted with the addition of PE 20 mL, and extracted with ACN 20 mL (10 mL*2). The combined PE layers are concentrated under reduced pressure to give compound A37 (47 mg, 28.04 μmol, 42.30% yield, 99% purity) as a colorless oil.

### 9.P3. Synthesis of A38-A44

Starting from commercially available reagents, A38-A44 are prepared following the procedure for the synthesis of A37, with minor modifications.

### 9.P4. Synthesis of 2370, 2544, 2545, 2547, 2551, 2577, 2578, 2580, 2581, 2582, 2583, 2584, 2585, 2586, 2587, 2588, 2589, 2590, 2591, 2599, 2600, 2601, 2608, 2610, 2611, 2613 and B1-B25

Starting from commercially available reagents, 2370, 2544, 2545, 2547, 2551, 2577, 2578, 2580, 2581, 2582, 2583, 2584, 2585, 2586, 2587, 2588, 2589, 2590, 2591, 2599, 2600, 2601, 2608, 2610, 2611, 2613 and B1-B25 are prepared following the procedure for the synthesis of 2481, with minor modifications.

### 9.P5. Synthesis of 2592

Starting from commercially available reagents, 2592 is prepared following the procedure for the synthesis of 2454, with minor modifications.

### Example 10. Preparation of Lipid Nanoparticle Compositions

### Exemplary Lipid Nanoparticle Compositions.

Exemplary lipid nanoparticle compositions were prepared to result in an ionizable lipid:structural lipid:sterol:PEG-lipid at a molar ratio shown in the below charts.

Molar ratios of the lipid components of each lipid nanoparticle composition are summarized below.

| | | Molar ratio | | | | |
|---|---|---|---|---|---|---|
| Ionizable | | Ionizable | | | Plant | PEG-Lipd |
| Lipid No. | mRNA | component | Structural | | Cholesterol | DMG-PEG2k |
| 2243 | FLUC/EPO 1:1 | 50 | DSPC | 10 | 38.5 | 1.5 |
| 2330 | FLUC/EPO 1:1 | 35 | DOPE | 16 | 46.5 | 2.5 |
| 2331 | FLUC/EPO 1:1 | 50 | DSPC | 10 | 38.5 | 1.5 |
| 2333 | FLUC/EPO 1:1 | 35 | DOPE | 16 | 46.5 | 2.5 |
| 2335 | FLUC/EPO 1:1 | 50 | DSPC | 10 | 38.5 | 1.5 |
| 2365 | FLUC/EPO 1:1 | 50 | DSPC | 10 | 38.5 | 1.5 |
| 2366 | FLUC/EPO 1:1 | 50 | DSPC | 10 | 38.5 | 1.5 |
| 2367 | FLUC/EPO 1:1 | 50 | DSPC | 10 | 38.5 | 1.5 |
| 2368 | FLUC/EPO 1:1 | 50 | DSPC | 10 | 38.5 | 1.5 |
| 2370 | FLUC/EPO 1:1 | 50 | DSPC | 10 | 38.5 | 1.5 |
| 2398 | FLUC/EPO 1:1 | 35 | DOPE | 16 | 46.5 | 2.5 |
| 2424 | FLUC/EPO 1:1 | 50 | DSPC | 10 | 38.5 | 1.5 |
| 2425 | FLUC/EPO 1:1 | 50 | DSPC | 10 | 38.5 | 1.5 |
| 2432 | FLUC/EPO 1:1 | 50 | DSPC | 10 | 38.5 | 1.5 |
| 2433 | FLUC/EPO 1:1 | 50 | DSPC | 10 | 38.5 | 1.5 |
| 2441 | FLUC/EPO 1:1 | 50 | DSPC | 10 | 38.5 | 1.5 |
| 2442 | FLUC/EPO 1:1 | 50 | DSPC | 10 | 38.5 | 1.5 |
| 2443 | FLUC/EPO 1:1 | 50 | DSPC | 10 | 38.5 | 1.5 |
| 2454 | FLUC/EPO 1:1 | 50 | DSPC | 10 | 38.5 | 1.5 |

To prepare the exemplary lipid nanoparticle compositions, the lipid components according to the above chart were solubilized in ethanol, mixed at the above-indicated molar ratios, and diluted in ethanol (organic phase) to obtain total lipid concentration of 5.5 mM.

Lipid Nanoparticle Compositions Encapsulating mRNA.

An mRNA solution (aqueous phase, fluc:EPO mRNA), according to the above chart for each LNP composition, was prepared with RNAse-free water and 100 mM citrate buffer pH 3 for a final concentration of 50 mM citrate buffer and 0.167 mg/mL mRNA concentration (1:1 Fluc:EPO). The formulations were maintained at an ionizable lipid to mRNA at an ionizable lipid nitrogen:mRNA phosphate (N:P) ratio of 6:1.

For each LNP composition, the lipid mix and mRNA solution were mixed at a 1:3 ratio by volume, respectively, on a NanoAssemblr Ignite (Precision Nanosystems) at a total flow rate of 9 mL/min. The resulting compositions were then loaded into Slide-A-Lyzer G2 dialysis cassettes (10k MWCO) and dialyzed in 200 times sample volume of 1×PBS for 2 hours at room temperature with gentle stirring. The PBS was refreshed, and the compositions were further dialyzed for at least 14 hours at 4° C., with gentle stirring. The dialyzed compositions were then collected and concentrated by centrifugation at 3000×g using Amicon Ultra centrifugation filters (100k MWCO). The concentrated particles were characterized for size, polydispersity, and particle concentration using Zetasizer Ultra (Malvern Panalytical) and for mRNA encapsulation efficiency using Quant-iT RiboGreen RNA Assay Kit (ThermoFisher Scientific).

For pKa measurement, a TNA assay was conducted according to those described in Sabnis et al., Molecular Therapy, 26(6):1509-19), which is incorporated herein by reference in its entirety. Briefly, 20 buffers (10 mM sodium phosphate, 10 mM sodium borate, 10 mM sodium citrate, and 150 mM sodium chloride, in distilled Water) of unique pH values ranging from 3.0-12.0 were prepared using IM sodium hydroxide and IM hydrochloric acid. 3.25 μL of a LNP composition (0.04 mg/mL mRNA, in PBS) was incubated with 2 μL of TNS reagent (0.3 mM, in DMSO) and 90 μL of buffer for each pH value (described above) in a 96-well black-walled plate. Each pH condition was performed in triplicate wells. The TNS fluorescence was measured using a Biotek Cytation Plate reader at excitation/emission wavelengths of 321/445 nm. The fluorescence values were then plotted and fit using a 4-parameter sigmoid curve. From the fit, the pH value yielding the half-maximal fluorescence was calculated and reported as the apparent LNP pKa value.

The particle characterization data for each exemplary lipid nanoparticle composition, labeled by the same ionizable lipid number based on which it was prepared, are shown in the table below.

| | | Size | | | pKa |
|---|---|---|---|---|---|
| LNP No. | mRNA | (nm) | PDI | % EE | (TNS) |
| 2243 | FLUC/EPO 1:1 | 112.5 | 0.018 | 97.3 | 7.10 |
| 2330 | FLUC/EPO 1:1 | 79.5 | 0.085 | 96.5 | 5.65 |
| 2331 | FLUC/EPO 1:1 | 90.0 | 0.045 | 97.4 | 5.41 |
| 2333 | FLUC/EPO 1:1 | 74.1 | 0.075 | 96.6 | 6.13 |
| 2335 | FLUC/EPO 1:1 | 87.2 | 0.050 | 97.5 | 6.40 |
| 2365 | FLUC/EPO 1:1 | 98.7 | 0.026 | 96.2 | 5.53 |
| 2366 | FLUC/EPO 1:1 | 113.7 | 0.078 | 93.1 | 5.21 |
| 2367 | FLUC/EPO 1:1 | 93.9 | 0.034 | 97.0 | 5.71 |
| 2368 | FLUC/EPO 1:1 | 93.5 | 0.028 | 96.2 | 5.10 |
| 2370 | FLUC/EPO 1:1 | 104.1 | 0.039 | 97.4 | 5.59 |
| 2398 | FLUC/EPO 1:1 | 130.8 | 0.210 | 87.6 | |
| 2424 | FLUC/EPO 1:1 | 114.5 | 0.059 | 98.5 | |
| 2425 | FLUC/EPO 1:1 | 124.8 | 0.039 | 98.8 | 4.80 |
| 2432 | FLUC/EPO 1:1 | 114.6 | 0.050 | 98.9 | |
| 2433 | FLUC/EPO 1:1 | 112.2 | 0.080 | 99.1 | 5.34 |
| 2441 | FLUC/EPO 1:1 | 140.4 | 0.081 | 99.3 | 5.11 |
| 2442 | FLUC/EPO 1:1 | 109.3 | 0.060 | 95.5 | 5.88 |
| 2443 | FLUC/EPO 1:1 | 108.6 | 0.179 | 95.2 | 6.21 |
| 2454 | FLUC/EPO 1:1 | 138.2 | 0.071 | 98.5 | 6.13 |

### Example 11. In-Vivo Bioluminescent Imaging

The exemplary lipid nanoparticle compositions prepared according to Example 10, with encapsulating an mRNA according to the table shown above in Example 9, were used in this example.

### Bioluminescence Screening.

8-9 week old female Balb/c mice were utilized for bioluminescence-based ionizable lipid screening efforts. Mice were obtained from Jackson Laboratories (JAX Stock: 000651) and allowed to acclimate for one week prior to manipulations. Animals were placed under a heat lamp for a few minutes before introducing them to a restraining chamber. The tail was wiped with alcohol pads (Fisher Scientific) and, for each LNP composition described above, 100 uL of a lipid nanoparticle composition described above containing 10 μg total mRNA (5 μg Fluc+5 μg EPO, 5 μg Fluc+5 μg Cre, or 5 μg EGFP) was injected intravenously using a 29G insulin syringe (Covidien), 4-6 hours post-dose, animals were injected with 200 μL of 15 mg/mL D-Luciferin (GoldBio), and placed in set nose cones inside the IVIS Lumina LT imager (PerkinElmer). LivingImage software was utilized for imaging. Whole body bio-luminescence was captured at auto-exposure after which animals are removed from the IVIS and placed into a CO₂ chamber for euthanasia. Cardiac puncture was performed on each animal after placing it in dorsal recumbency, and blood collection was performed using a 25G insulin syringe (BD). Once all blood samples were collected, tubes are spun at 2000G for 10 minutes using a tabletop centrifuge and plasma was aliquoted into individual Eppendorf tubes (Fisher Scientific) and stored at −80° C. for subsequent EPO quantification. EPO levels in plasma were determined using EPO MSD kit (Meso Scale Diagnostics). The hEPO MSD measurement protocol was the same as those described in Section hEPO MSD Measurement in Example 8.

The EPO levels determined by the in-vivo bioluminescent imaging for each lipid nanoparticle compositions are shown in the table below.

| LNP | | Whole | | | | | Spleen:Liver |
|---|---|---|---|---|---|---|---|
| No. | mRNA dose | Body | Liver | Spleen | Lung | hEPO | Ratio |
| 2243 | 5 ug FLUC + | 8.75E+07 | 1.11E+07 | 2.46E+07 | 5.23E+05 | 7.14E+05 | 2.61E+00 |
| | 5 ug EPO | | | | | | |
| 2330 | 5 ug FLUC + | 4.78E+07 | 8.99E+06 | 5.54E+04 | 1.41E+03 | 3.62E+05 | 5.98E−03 |
| | 5 ug EPO | | | | | | |
| 2331 | 5 ug FLUC + | 6.75E+07 | 7.77E+06 | 3.46E+06 | — | — | 4.92E−01 |
| | 5 ug EPO | | | | | | |
| 2333 | 5 ug FLUC + | 1.21E+09 | 7.11E+07 | 6.63E+05 | 1.18E+04 | — | 9.45E−03 |
| | 5 ug EPO | | | | | | |
| 2335 | 5 ug FLUC + | 7.56E+07 | 5.93E+06 | 3.77E+06 | 3.17E+04 | 1.23E+06 | 6.40E−01 |
| | 5 ug EPO | | | | | | |
| 2365 | 5 ug FLUC + | 6.60E+07 | 1.07E+07 | 3.74E+07 | 6.90E+04 | 5.20E+05 | 4.90E+00 |
| | 5 ug EPO | | | | | | |
| 2366 | 5 ug FLUC + | 3.41E+07 | 4.28E+06 | 5.42E+06 | 3.65E+04 | 4.28E+06 | 1.44E+00 |
| | 5 ug EPO | | | | | | |
| 2367 | 5 ug FLUC + | 2.33E+08 | 4.93E+07 | 6.17E+06 | 2.41E+04 | 2.77E+06 | 1.77E−01 |
| | 5 ug EPO | | | | | | |
| 2368 | 5 ug FLUC + | 9.14E+04 | 8.50E+03 | 2.21E+04 | 8.34E+02 | 1.13E+03 | 2.60E+00 |
| | 5 ug EPO | | | | | | |
| 2370 | 5 ug FLUC + | 4.67E+04 | 5.92E+03 | 3.67E+04 | 5.17E+02 | 6.64E+02 | 6.49E+00 |
| | 5 ug EPO | | | | | | |
| 2398 | 5 ug FLUC + | 1.29E+07 | 2.13E+06 | 1.81E+04 | 1.88E+03 | 3.96E+04 | 8.29E−03 |
| | 5 ug EPO | | | | | | |
| 2424 | 5 ug FLUC + | 4.21E+08 | 6.17E+07 | 1.07E+07 | 1.36E+05 | 4.02E+06 | 2.10E−01 |
| | 5 ug EPO | | | | | | |
| 2425 | 5 ug FLUC + | 1.55E+08 | 1.95E+07 | 2.13E+07 | 1.75E+04 | 1.01E+06 | 1.25E+00 |
| | 5 ug EPO | | | | | | |
| 2432 | 5 ug FLUC + | 2.63E+07 | 4.90E+06 | 7.19E+05 | 5.37E+03 | 9.36E+05 | 1.79E−01 |
| | 5 ug EPO | | | | | | |
| 2433 | 5 ug FLUC + | 5.24E+07 | 9.33E+06 | 9.43E+06 | 1.45E+04 | 5.78E+05 | 1.12E+00 |
| | 5 ug EPO | | | | | | |
| 2441 | 5 ug FLUC + | 4.29E+05 | 2.83E+04 | 7.83E+04 | 4.40E+03 | 1.04E+05 | 3.51E+00 |
| | 5 ug EPO | | | | | | |
| 2442 | 5 ug FLUC + | 7.08E+08 | 1.03E+08 | 4.43E+06 | 2.03E+04 | 7.44E+06 | 5.57E−02 |
| | 5 ug EPO | | | | | | |
| 2443 | 5 ug FLUC + | 2.83E+04 | 3.85E+03 | 1.48E+04 | 3.03E+02 | 2.28E+04 | 3.85E+00 |
| | 5 ug EPO | | | | | | |
| 2454 | 5 ug FLUC + | 9.43E+07 | 1.81E+07 | 1.35E+07 | 9.33E+04 | 5.63E+05 | 8.75E−01 |
| | 5 ug EPO | | | | | | |

As can be seen in the table above, the lipid nanoparticle compositions containing the novel ionizable lipid compounds demonstrated an effective delivery of the therapeutic cargos in the whole body, as well as various organs such as liver, spleen, and lung.

Some of the exemplary lipid nanoparticle compositions demonstrated a selective delivery of the therapeutic cargos outside the liver and, due to the lower lipid levels in the liver, lower liver toxicity is expected. In particular, the spleen:liver ratio of average radiance was determined for all the exemplary lipid nanoparticle compositions. Almost half of the exemplary lipid nanoparticle compositions (9 out of 19) exhibited a spleen to liver ratio of >1. These results indicate that many of these exemplary lipid nanoparticle compositions exhibited high delivery to spleen delivery in addition to liver delivery.

While this disclosure has been described in relation to some embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications, and equivalents. In particular, this disclosure includes any combination of the features, terms, or elements of the various illustrative components and examples.

1. A lipid comprising at least one head group and at least one tail group of formula (TI) or (TI′) 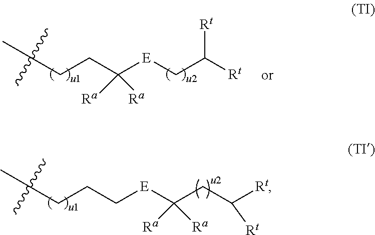
a pharmaceutically acceptable salt thereof, or a stereoisomer of any of the foregoing,
wherein:
E is each independently —OC(O)—, —C(O)O—, —N(R⁷)C(O)—, —C(O)N(R⁷)—, —C(O—R₁₃)—O—, —C(O)O(CH₂)ᵣ—, —C(O)N(R⁷)(CH₂)ᵣ—, —S—S—, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl; and r is 1, 2, 3, 4, or 5;
R^{a} is each independently C₁-C₅alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl; [symbol]
represents the bond connecting the tail group to the head group; and
wherein the lipid has a pKa from about 4 to about 8.

2-3. (canceled)

4. The lipid of claim 1, wherein the lipid comprises at least one tail group of the following formulas: 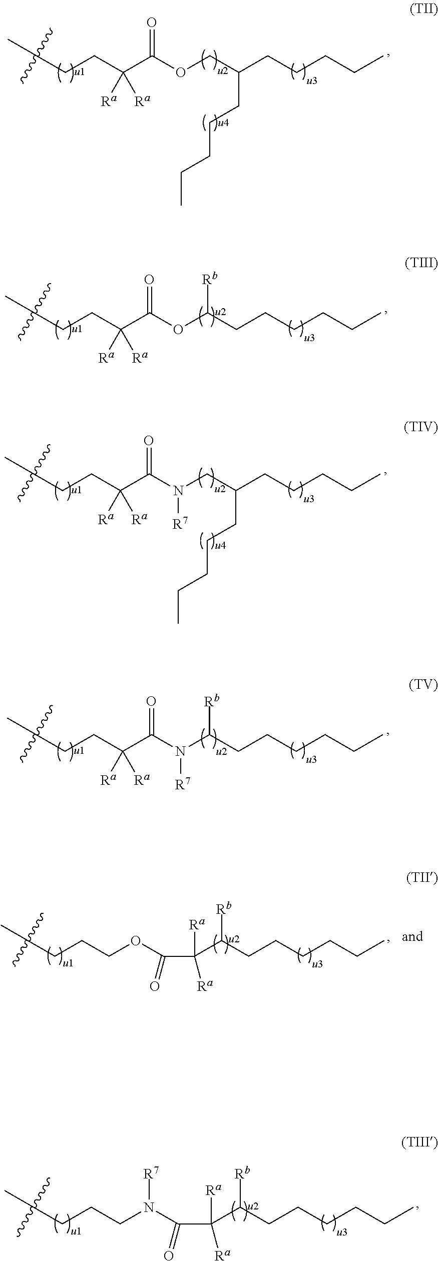
wherein
R⁷ is each independently H or methyl;
R^{b} is in each occasion independently H or C₁-C₄ alkyl; and
u3 and u4 are each independently 0, 1, 2, 3, 4, 5, 6, or 7, and
wherein the lipid has a pKa from about 4 to about 8.

5-6. (canceled)

7. The lipid of claim 4, wherein the lipid comprises two, three, four, or more tail groups of formula (TII), (TIII), (TIV), (TV), (TII′), or (TIII′), and wherein each tail group may be the same or different.

8. The lipid of claim 1, wherein:
each R^{a} is methyl; and/or
u1 is 3-5, u2 is 0-3, and u3 and u4 are each independently 1-7.

9. (canceled)

10. The lipid of claim 4, wherein the lipid comprises:
(i) at least one tail of formula (TIII), wherein each R^{a} is methyl, R^{b} is in each occasion independently H, ethyl, or butyl, u1 is 3-5, u2 is 0-3, and u3 is 1-7;
(ii) at least one tail of formula (TII), wherein each R^{a} is methyl, u1 is 3-5, u2 is 0-3, u3 is 1-4, and u4 is 1-4;
(iii) at least one tail of formula (TII) and at least one tail of formula (TIII);
(iv) at least one tail of formula (TIV), wherein each R^{a} is methyl, u1 is 3-5, u2 is 0-3, u3 is 1-4, and u4 is 1-4;
(v) at least two, three, or four tails of formula (TV), wherein the two, three, or four tails of formula (TV) are the same or different;
(vi) at least two, three, or four tails of formula (TII′), wherein the two, three, or four tails of formula (TII′) are the same or different;
(vii) at least two, three, or four tails of formula (TIII′), wherein the two, three, or four tails of formula (TIII′) are the same or different;
(viii) at least one tail selected from the group consisting of formula (TII), (TIII), and (TII′), and at least one tail selected from the group consisting of (TIV), (TV), and (TIII′);
(ix) at least two tails selected from the group consisting of (TII), (TIII), and (TII′);
(x) at least two tails selected from the group consisting of (TIV), (TV), and (TIII′);
(xi) at least two tails selected from the group consisting of formula (TII), (TIII), and (TII′), and at least one tail selected from the group consisting of (TIV), (TV), and (TIII′);
(xii) at least one tail selected from the group consisting of formula (TII), (TIII), and (TII′), and at least two tails selected from the group consisting of (TIV), (TV), and (TIII′);
(xiii) at least two tails selected from the group consisting of formula (TII), (TIII), and (TII′), and at least two tails selected from the group consisting of (TIV), (TV), and (TIII′);
(xiv) at least three tails selected from the group consisting of (TII), (TIII), and (TII′);
(xv) at least three tails selected from the group consisting of (TIV), (TV), and (TIII′);
(xvi) at least three tails selected from the group consisting of formula (TII), (TIII), and (TII′), and at least one tail selected from the group consisting of (TIV), (TV), and (TIII′);
(xvii) at least one tail selected from the group consisting of formula (TII), (TIII), and (TII′), and at least three tails selected from the group consisting of (TIV), (TV), and (TIII′);
(xviii) at least two tails of formula (TII) or (TIII), and at least two tails of formula (TIV) or (TV);
(xix) at least two tails of formula (TII) or (TIII), and at least two tails of formula (TII′) or (TIII′); or
(xx) at least two tails of formula (TIV) or (TV), and at least two tails of formula (TII′) or (TIII′).

11-34. (canceled)

35. The lipid of claim 1, further comprising at least one tail of formula (TNG-I): 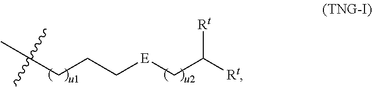
wherein
E is —OC(O)—, —C(O)O—, —N(R⁷)C(O)—, —S—S—, or —C(O)N(R⁷)—;
u1 and u2 are each independently 0, 1, 2, 3, 4, 5, 6, or 7; and
R⁷ is independently H, alkyl, alkenyl, cycloalkyl, hydroxyalkyl, or aminoalkyl.

36-38. (canceled)

39. The lipid of claim 1, wherein the head group has a structure of one of the following formulas: 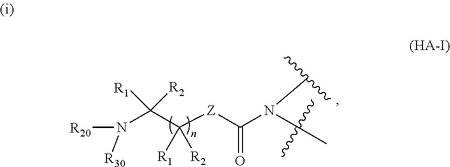
wherein:
R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
R₂₀ and R₃₀, together with the adjacent N atom, form a 3 to 7 membered heterocylic or heteroaromatic ring containing one or more heteroatoms, optionally substituted with one or more OH, SH, halogen, alkyl, or cycloalkyl groups;
each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
each of R₁₀ and R₁₁ is independently H, C₁-C₃ branched or unbranched alkyl, C₂-C₃ branched or unbranched alkenyl; or R₁₀ and R₁₁ together form a heterocyclic ring;
n is 0, 1, 2, 3 or 4;
Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, SH; 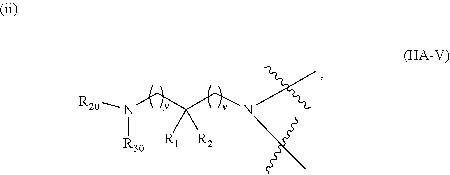
wherein:
R₁ is H, C₁-C₃ alkyl, OH, halogen, SH, or NR₁₀R₁₁;
R₂ is OH, halogen, SH, or NR₁₀R₁₁; or R₁ and R₂ can be taken together to form a cyclic ring;
R₁₀ and R₁₁ are each independently H or C₁-C₃ alkyl; or R₁₀ and R₁₁ can be taken together to form a heterocyclic ring;
R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
each of v and y is independently 1, 2, 3, or 4; 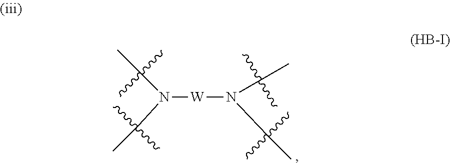
wherein W is 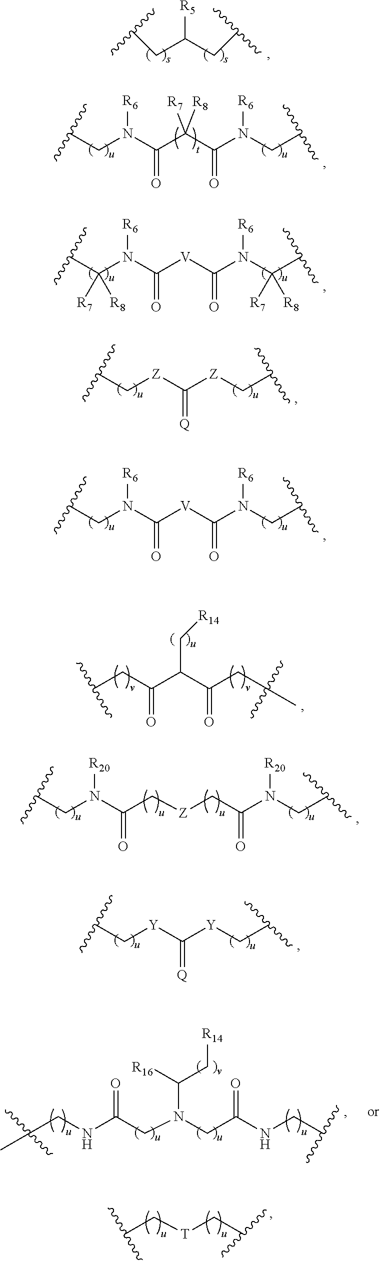
wherein
R₅ is OH, SH, (CH₂)ₛOH, or NR₁₀R₁₁;
each R₆ is independently H, C₁-C₃ branched or unbranched alkyl, C₂-C₃ branched or unbranched alkenyl, or cycloalkyl;
each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or R₇ and R₈ are taken together to form a ring;
each R₂₀ is independently H, or C₁-C₃ branched or unbranched alkyl;
R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
R₁₆ is H, ═O, ═S, or CN;
each of s, u, and t is independently 1, 2, 3, 4, or 5;
each v is independently 0, 1, 2, 3, 4, or 5;
each Y is a divalent heterocyclic;
each Z is independently absent, O, S, or NR₁₂, wherein R₁₂ is H, C₁-C₇ branched or unbranched alkyl, or C₂-C₇ branched or unbranched alkenyl;
Q is O, S, CH₂, or NR₁₃, wherein each R₁₃ is H, C₁-C₅alkyl;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
T is —NHC(O)O—, —OC(O)NH—, or a divalent heterocyclic; or 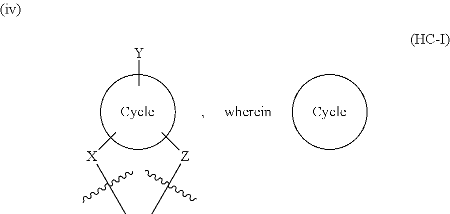
is selected from the group consisting of 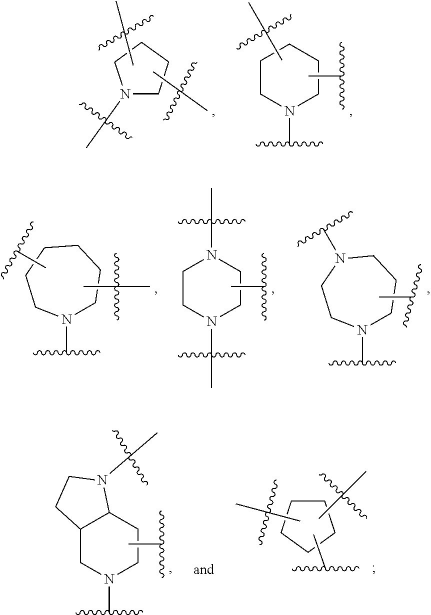
Y is alkyl, hydroxy, hydroxyalkyl, 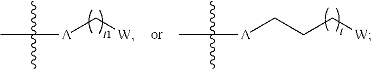
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, or —S—S—;
each of X and Z is independently absent, —O—, —C(O)—, —N(R⁷)-alkylene, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t is 0, 1, 2, or 3;
t1 is an integer from 0 to 10; and
W is hydroxyl, substituted or unsubstituted hydroxyalkyl, or one of the following moieties: 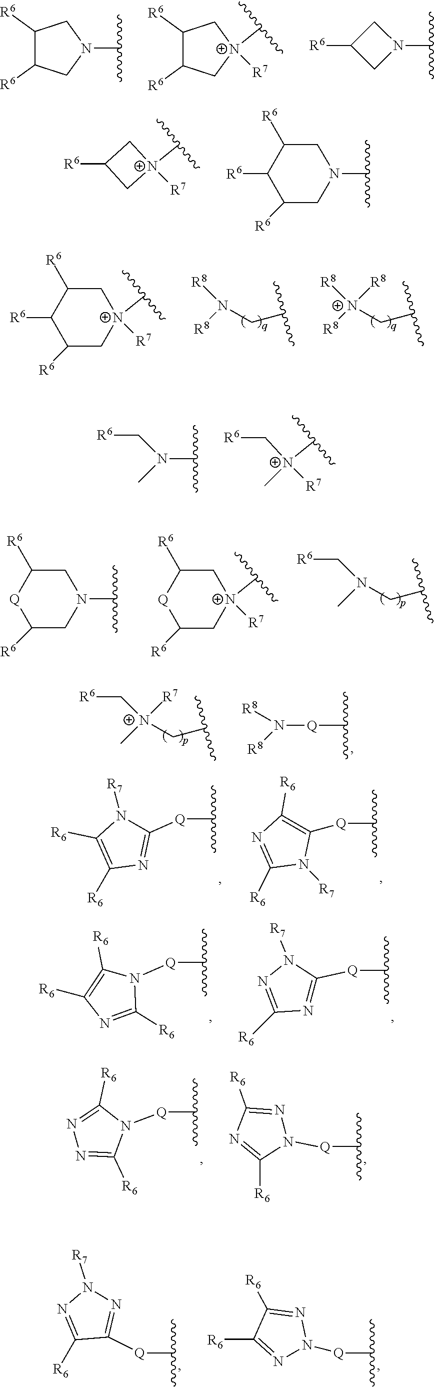 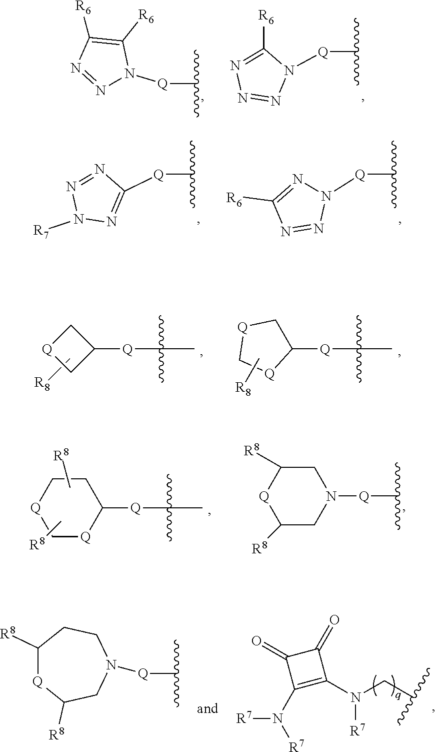
wherein:
each Q is independently absent, —O—, —C(O)—, —C(S)—, —C(O)O—, —(CH₂)_{q}—C(R⁷)₂, —C(O)N(R⁷)—, —C(S)N(R⁷)—, or —N(R⁷);
R⁶ is independently H, alkyl, hydroxyl, hydroxyalkyl, alkoxy, —O-alkylene-O-alkyl, —O-alkylene-N(R⁷)₂, amino, alkylamino, aminoalkyl, thiol, thiolalkyl, or N⁺ (R⁷)₃-alkylene-Q-;
each R⁸ is independently H, alkyl, hydroxyalkyl, amino, aminoalkyl, alkylamino, thiol, thiolalkyl, heterocyclyl, heteroaryl; or two R⁸ together with the nitrogen atom form a ring, optionally substituted with one or more alkyl, hydroxy, hydroxyalkyl, alkoxy, alkylaminoalkyl, alkylamino, or aminoalkyl;
q is 0, 1, 2, 3, 4, or 5; and
p is 0, 1, 2, 3, 4, or 5.

40. The lipid of claim 39, wherein the head group has a structure of (i) formula (HA-I), and wherein:
R₂₀ and R₃₀ together with the adjacent N atom form a 3 to 7 membered heterocylic or heteroaromatic ring containing one or more heteroatoms, optionally substituted with one or more OH, SH, halogen, alkyl, or cycloalkyl groups;
Z is absent, O, S, or NH;
each R₁ and R₂ are H; and/or
n is 0, 1, or 2.

41-43. (canceled)

44. The lipid of claim 40, wherein the head group has a structure of: 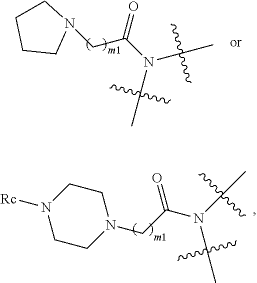
wherein:
Rc is H, alkyl, or alkyl substituted with OH; and
m1 is 1, 2, or 3.

45. (canceled)

46. The lipid of claim 39, wherein the head group has a structure of (ii) formula (HA-V), having a structure of formula (HA-VI): 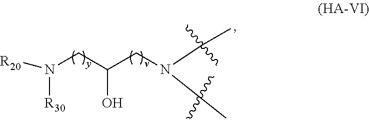
wherein each R₂₀ and R₃₀ are independently C₁-C₃ alkyl.

47-48. (canceled)

49. The lipid of claim 39, wherein the head group has a structure of (iii) formula (HB-I), and wherein:
R₅ is OH or (CH₂)ₛOH; and s is 1 or 2;
each R₆, R₇, and R₈ are independently H or C₁-C₃ alkyl;
each of u and t is independently 1, 2, or 3;
each v is independently 0, 1, 2, or 3;
R₁₆ is H or ═O;
each Z is independently absent, O, or NR₁₂, wherein R₁₂ is H or C₁-C₃ alkyl;
T is a divalent heterocylic;
Q is O or CH₂; and
V is C₂-C₆ alkylene or C₂-C₆ alkenylene.

50. The lipid of claim 49, wherein W is 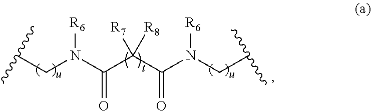
wherein:
each R₆, R₇, and R₈ are independently H or methyl; and
each of u and t is independently 1, 2, or 3; 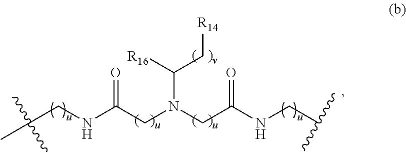
wherein:
R₁₆ is H or ═O;
R₁₄ is a nitrogen-containing 5- or 6-membered heterocyclic NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H or C₁-C₃ alkyl; and
each of u and v is independently 1, 2, or 3; 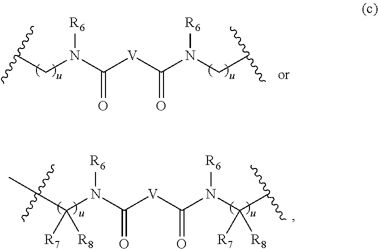
wherein:
each R₆ is independently H or methyl;
each R₇ is independently H;
each R₈ is methyl;
each u is independently 1, 2, or 3; and
V is C₂-C₆ alkylene or C₂-C₆ alkenylene; or 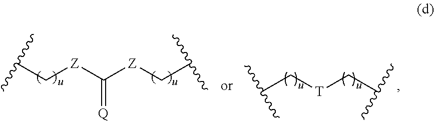
wherein:
each u is independently 1, 2, or 3;
Q is O;
each Z is independently NR₁₂;
R₁₂ is H or C₁-C₃ alkyl; and
T is a divalent nitrogen-containing 5- or 6-membered heterocyclic.

51-53. (canceled)

54. The lipid of claim 39, wherein the head group has the structure of: 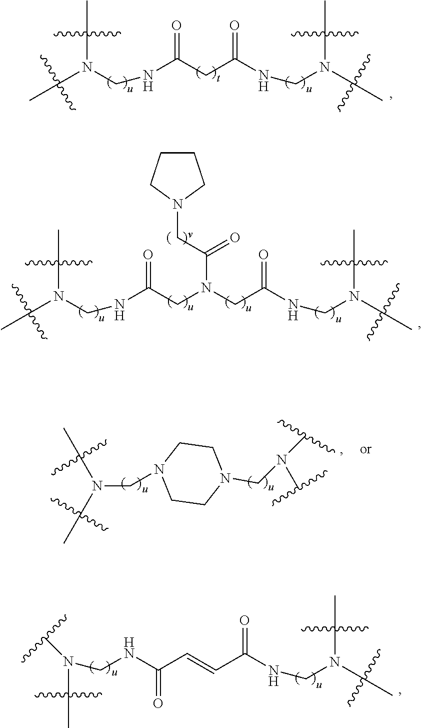
wherein each of u and t is independently 1 or 2.

55-56. (canceled)

57. The lipid of claim 39, wherein the head group has a structure of (iv) formula (HC-I), having a structure of formula (HC-IA): 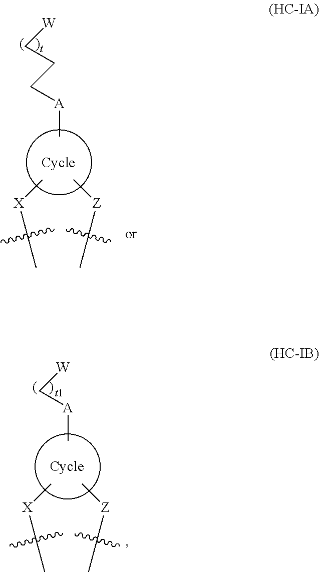
wherein W is one of the following: 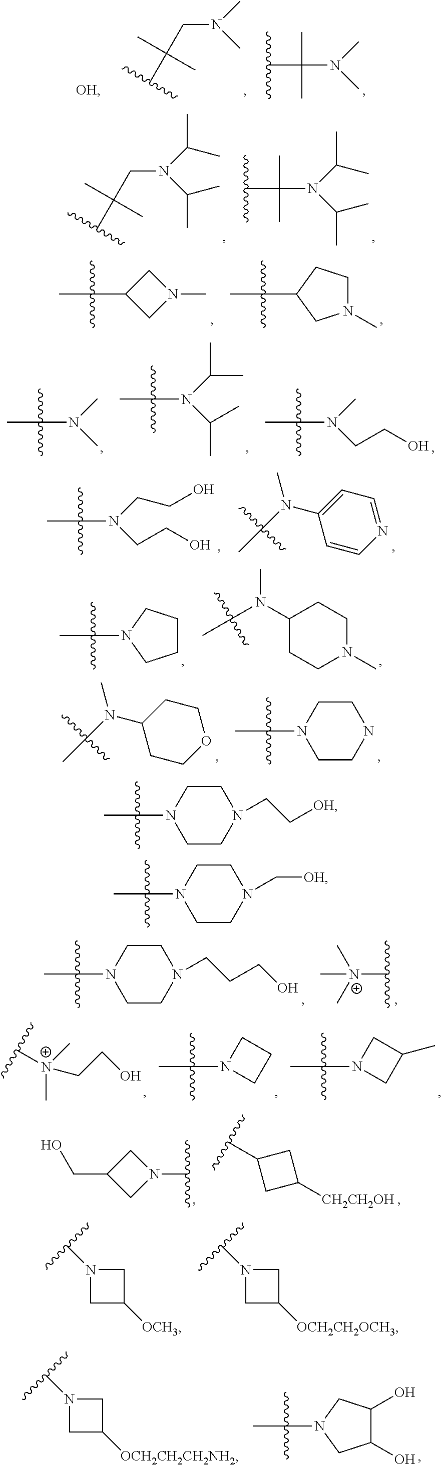 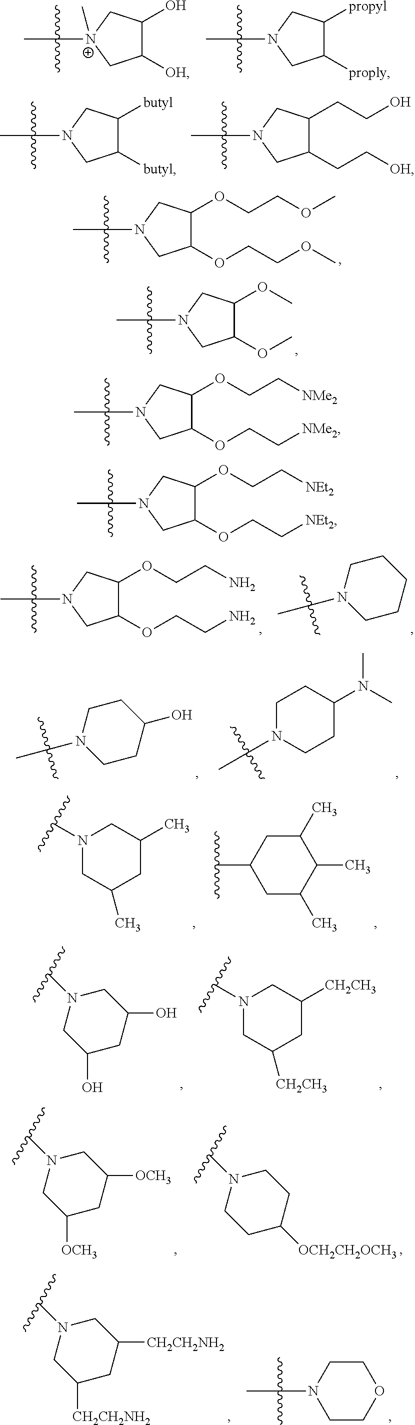 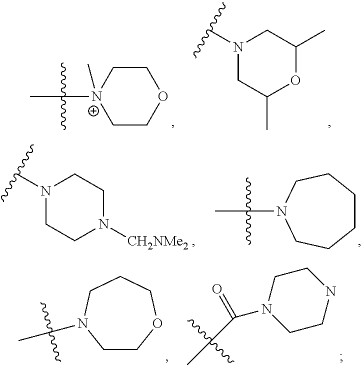
and/or
wherein:
A is absent, —O—, —N(R⁷)—, —OC(O)—, or —C(O)O—;
X is absent, —O—, or —C(O)—; and
Z is —O—, —C(O)O—, or —OC(O)—.

58-61. (canceled)

62. The lipid of claim 57, wherein the head group has a structure of formula

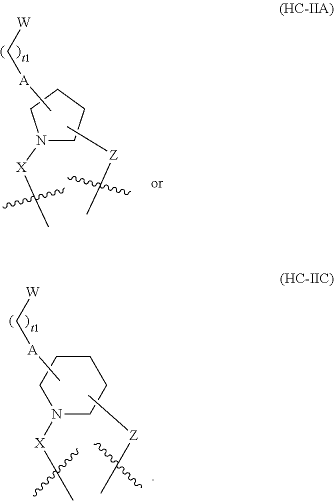

63-64. (canceled)

65. The lipid of claim 62, wherein the head group has a structure of formula 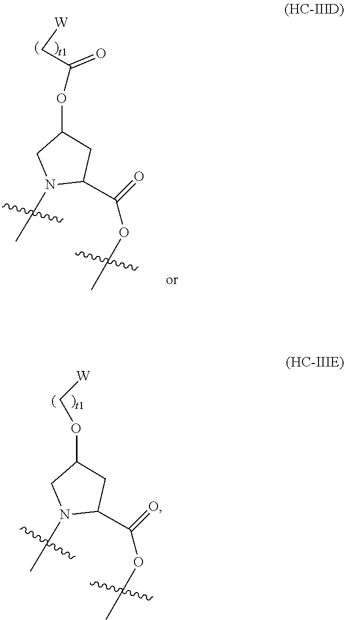
wherein t1 is 0, 1, 2, or 3.

66. (canceled)

67. A lipid comprising at least one head group and at least one tail group, wherein:
at least one tail group has a structure of formula (TI) or (TI′) 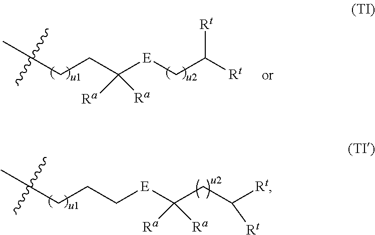
wherein:
E is each independently a biodegradable group;
R^{a} is each independently C₁-C₅alkyl, C₂-C₅ alkenyl, or C₂-C₅ alkynyl;
u1 and u2 are each independently 0, 1, 2, 3, 4, 5, 6, or 7;
R^{t} is each independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl; and [symbol]
represents the bond connecting the tail group to the head group; and
the head group has a structure of one of the following formulas:
i) 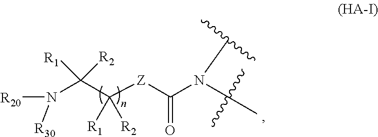
wherein:
R₂₀ and R₃₀ are each independently H, C₁-C₅ branched or unbranched alkyl, or C₂-C₅ branched or unbranched alkenyl, optionally interrupted with one or more heteroatoms or substituted with OH, SH, halogen, or cycloalkyl groups; or
R₂₀ and R₃₀, together with the adjacent N atom, form a 3 to 7 membered heterocylic or heteroaromatic ring containing one or more heteroatoms, optionally substituted with one or more OH, SH, halogen, alkyl, or cycloalkyl groups;
each of R₁ and R₂ is independently H, C₁-C₃ branched or unbranched alkyl, C₂-C₃ branched or unbranched alkenyl, OH, halogen, SH, or NR₁₀R₁₁; or R₁ and R₂ together form a cyclic ring;
each of R₁₀ and R₁₁ is independently H, C₁-C₃ branched or unbranched alkyl, C₂-C₃ branched or unbranched alkenyl; or R₁₀ and R₁₁ together form a heterocyclic ring;
n is 0, 1, 2, 3 or 4; and
Z is absent, O, S, or NR₁₂, wherein R₁₂ is H or C₁-C₇ branched or unbranched alkyl; provided that when Z is not absent, the adjacent R₁ and R₂ cannot be OH, NR₁₀R₁₁, SH;
ii) 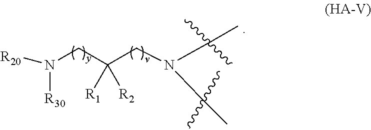
wherein:
R₁ is H, C₁-C₃ alkyl, OH, halogen, SH, or NR₁₀R₁₁;
R₂ is OH, halogen, SH, or NR₁₀R₁₁; or R₁ and R₂ can be taken together to form a cyclic ring;
R₁₀ and R₁₁ are each independently H or C₁-C₃ alkyl; or R₁₀ and R₁₁ can be taken together to form a heterocyclic ring;
R₂₀ and R₃₀ are each independently H, C₁-C₅ branched or unbranched alkyl, C₂-C₅ branched or unbranched alkenyl; or R₂₀ and R₃₀ can be taken together to form a cyclic ring; and
each of v and y is independently 1, 2, 3, or 4;
iii) 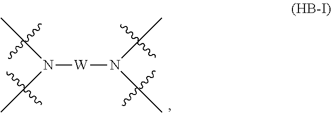
wherein W is 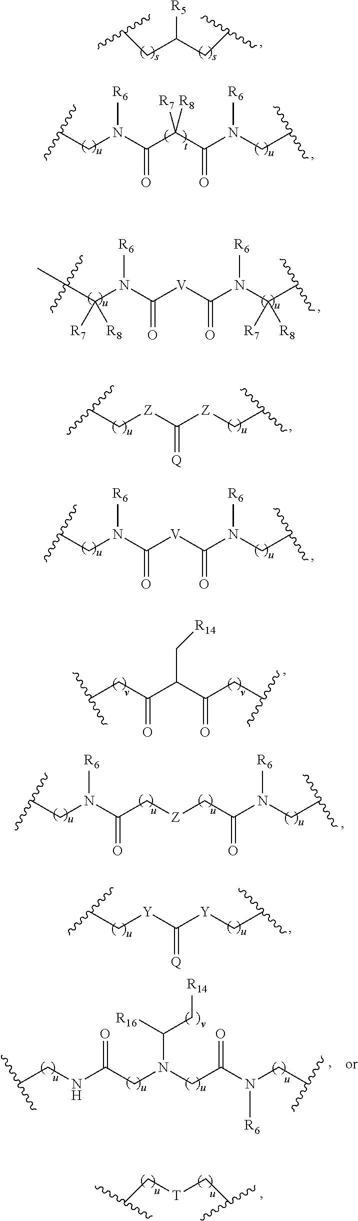
wherein
R₅ is OH, SH, (CH₂)ₛOH, or NR₁₀R₁₁;
each R₆ is independently H, C₁-C₃ branched or unbranched alkyl, C₂-C₃ branched or unbranched alkenyl, or cycloalkyl;
each R₇ and R₈ are independently H, C₁-C₃ branched or unbranched alkyl, C₂-C₃ branched or unbranched alkenyl, halogen, (CH₂)ᵥOH, (CH₂)ᵥSH, (CH₂)ₛN(CH₃)₂, or NR₁₀R₁₁, wherein each R₁₀ and R₁₁ is independently H or C₁-C₃ alkyl, or R₁₀ and R₁₁ are taken together to form a heterocyclic ring; or R₇ and R₈ are taken together to form a ring;
each R₂₀ is independently H, or C₁-C₃ branched or unbranched alkyl;
R₁₄ is a heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H, C₁-C₃ alkyl, C₃-C₇ cycloalkyl, C₃-C₇ cycloalkenyl, optionally substituted with one or more NH and/or oxo groups, or R₁₀ and R₁₁ are taken together to form a heterocyclic ring;
R₁₆ is H, ═O, ═S, or CN;
each of s, u, and t is independently 1, 2, 3, 4, or 5;
each v is independently 0, 1, 2, 3, 4, or 5;
each Y is a divalent heterocyclic;
each Z is independently absent, O, S, or NR₁₂, wherein R₁₂ is H, C₁-C₇ branched or unbranched alkyl, or C₂-C₇ branched or unbranched alkenyl;
Q is O, S, CH₂, or NR₁₃, wherein each R₁₃ is H, C₁-C₅alkyl;
V is branched or unbranched C₂-C₁₀ alkylene, C₂-C₁₀ alkenylene, C₂-C₁₀ alkynylene, or C₂-C₁₀ heteroalkylene, optionally substituted with one or more OH, SH, and/or halogen groups; and
T is —NHC(O)O—, —OC(O)NH—, or a divalent heterocyclic; and
iv) 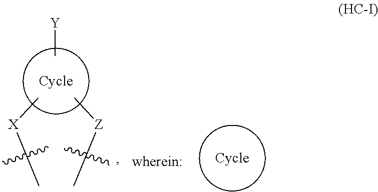
is cyclic or heterocyclic moiety;
Y is alkyl, hydroxy, hydroxyalkyl, 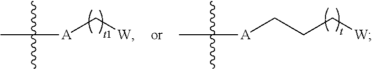
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, or —S—S—;
each of X and Z is independently absent, —O—, —C(O)—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, alkoxy, hydroxyalkyl, alkylamino, alkylaminoalkyl, or aminoalkyl;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl; and
wherein the lipid has a pKa from about 4 to about 8.

68-69. (canceled)

70. The lipid of claim 67, wherein at least one tail group has the structure of at least one of the following formulas: 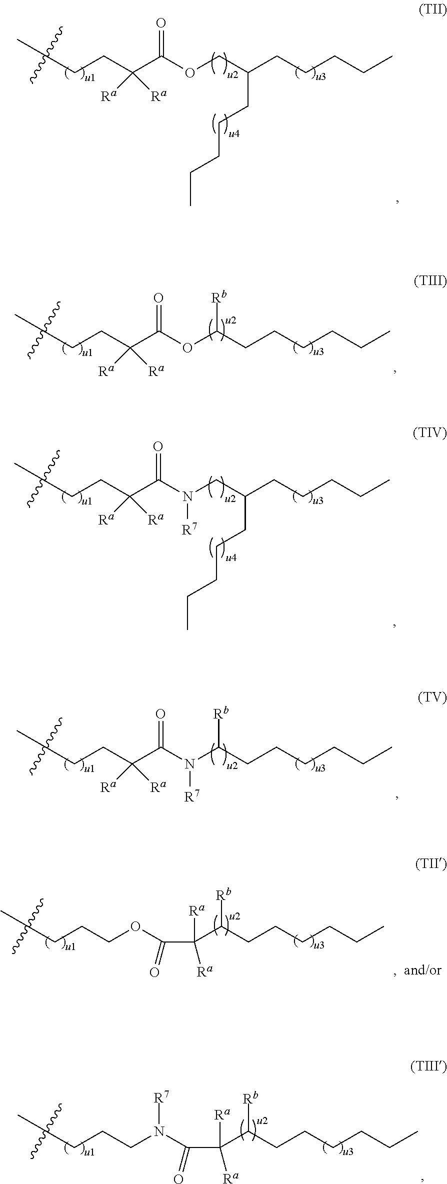
wherein each R^{a} is methyl; u1 is 3-5, u2 is 0-3; and u3 and u4 are each independently 1-7; and
wherein the head group has the structure of one of the following formulas 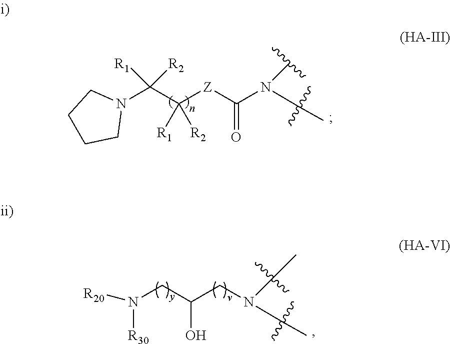
wherein each R₂₀ and R₃₀ are independently C₁-C₃ alkyl. 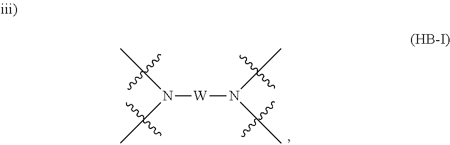
wherein:
W is 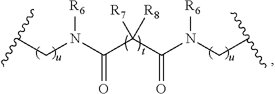
wherein:
each R₆, R₇, and R₈ are independently H or methyl; and
each of u and t is independently 1, 2, or 3; or
W is 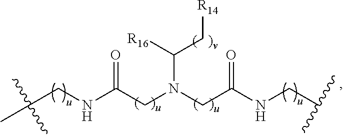
wherein:
R₁₆ is H or ═O;
R₁₄ is a nitrogen-containing 5- or 6-membered heterocyclic, NR₁₀R₁₁, C(O)NR₁₀R₁₁, NR₁₀C(O)NR₁₀R₁₁, or NR₁₀C(S)NR₁₀R₁₁, wherein each R₁₀ and R₁₁ is independently H or C₁-C₃ alkyl; and
each of u and v is independently 1, 2, or 3; or
W is 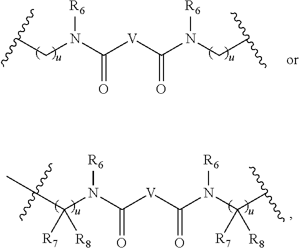
wherein:
each R₆ is independently H or methyl;
each R₇ is independently H;
each R₈ is methyl;
each u is independently 1, 2, or 3; and
V is C₂-C₆ alkylene or C₂-C₆ alkenylene; or
W is 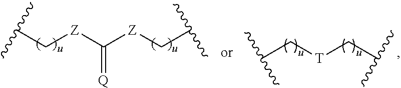
wherein:
each u is independently 1, 2, or 3;
Q is O;
each Z is independently NR₁₂; and
T is a divalent nitrogen-containing 5- or 6-membered heterocyclic; and
iv) 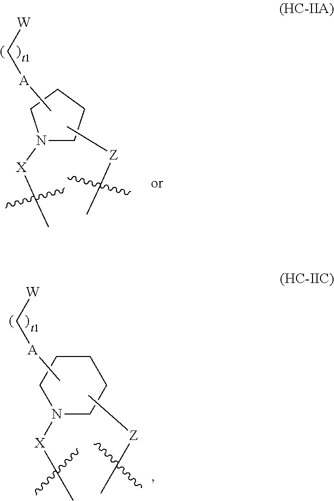
wherein:
W is hydroxyl, substituted or unsubstituted hydroxyalkyl, one of the following moieties: 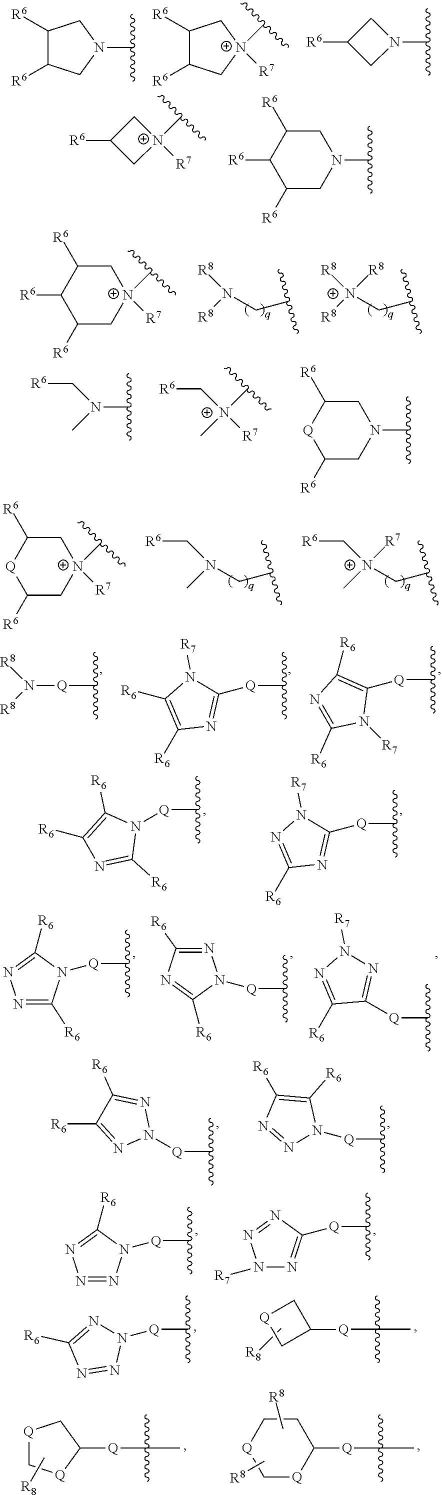 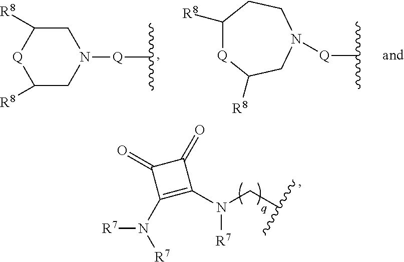
wherein
each Q is independently absent, —O—, —C(O)—, —C(S)—, —C(O)O—, —(CH₂)_{q}—C(R⁷)₂—, —C(O)N(R⁷)—, —C(S)N(R⁷)—, or —N(R⁷);
R⁶ is independently H, alkyl, hydroxyl, hydroxyalkyl, alkoxy, —O-alkylene-O-alkyl, —O-alkylene-N(R⁷)₂, amino, alkylamino, aminoalkyl, thiol, thiolalkyl, or N⁺(R⁷)₃-alkylene-Q-;
each R⁸ is independently H, alkyl, hydroxyalkyl, amino, aminoalkyl, alkylamino, thiol, thiolalkyl, heterocyclyl, heteroaryl; or two R⁸ together with the nitrogen atom form a ring, optionally substituted with one or more alkyl, hydroxy, hydroxyalkyl, alkoxy, alkylaminoalkyl, alkylamino, or aminoalkyl;
q is 0, 1, 2, 3, 4, or 5; and
p is 0, 1, 2, 3, 4, or 5.

71. The lipid of claim 67, having one of the following structures:

| | |
|---|---|
| 2243 | 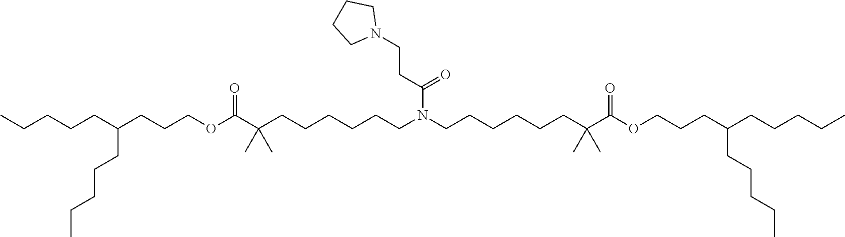 [SMILES: CCCCCC(CCCCC)CCCOC(=O)C(C)(C)CCCCCCN(CCCCCCC(C)(C)C(=O)OCCCC(CCCCC)CCCCC)C(=O)CCN1CCCC1] |
| 2330 | 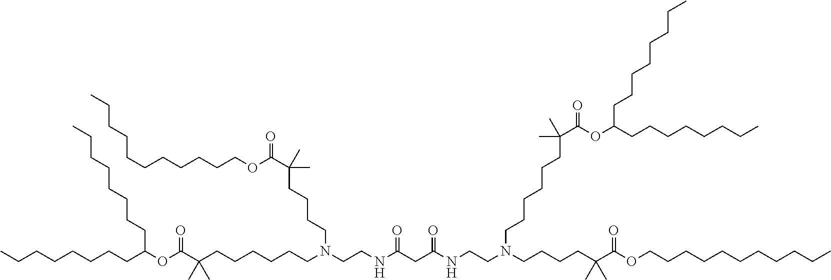 [SMILES: CCCCCCCCCCCOC(=O)C(C)(C)CCCCN(CCCCCCC(C)(C)C(=O)OC(CCCCCCCC)CCCCCCCC)CCNC(=O)CC(=O)NCCN(CCCCCCC(C)(C)C(=O)OC(CCCCCCCC)CCCCCCCC)CCCCC(C)(C)C(=O)OCCCCCCCCCCC] |
| 2331 | 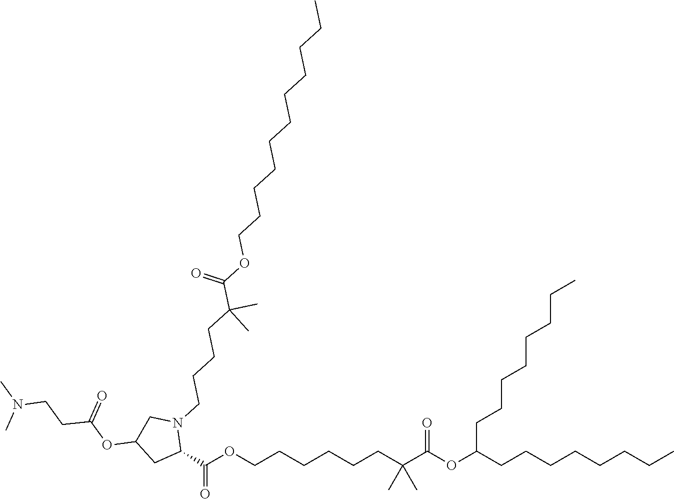 [SMILES: CCCCCCCCCCCOC(=O)C(C)(C)CCCCN1CC(OC(=O)CCN(C)C)C[C@H]1C(=O)OCCCCCCC(C)(C)C(=O)OC(CCCCCCCC)CCCCCCCC] |
| 2333 | 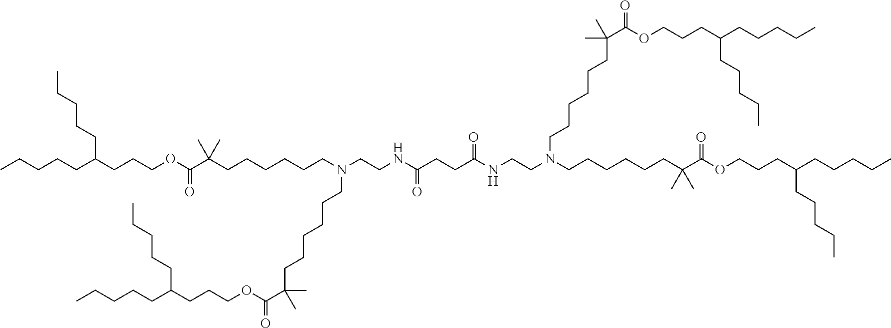 [SMILES: CCCCCC(CCCCC)CCCOC(=O)C(C)(C)CCCCCCN(CCCCCCC(C)(C)C(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] |
| 2335 | 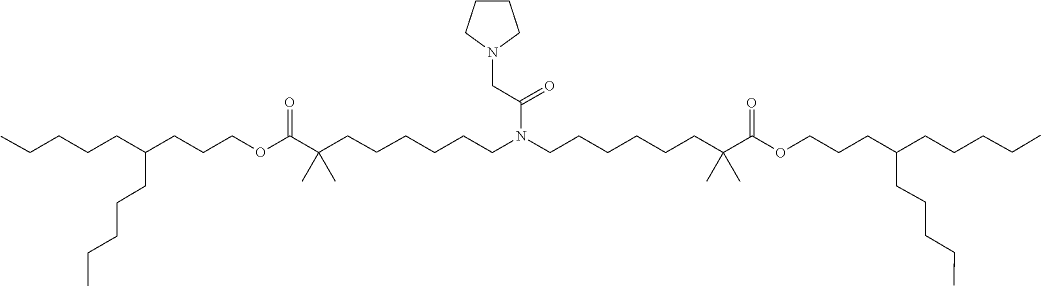 [SMILES: CCCCCC(CCCCC)CCCOC(=O)C(C)(C)CCCCCCN(CCCCCCC(C)(C)C(=O)OCCCC(CCCCC)CCCCC)C(=O)CN1CCCC1] |
| 2365 | 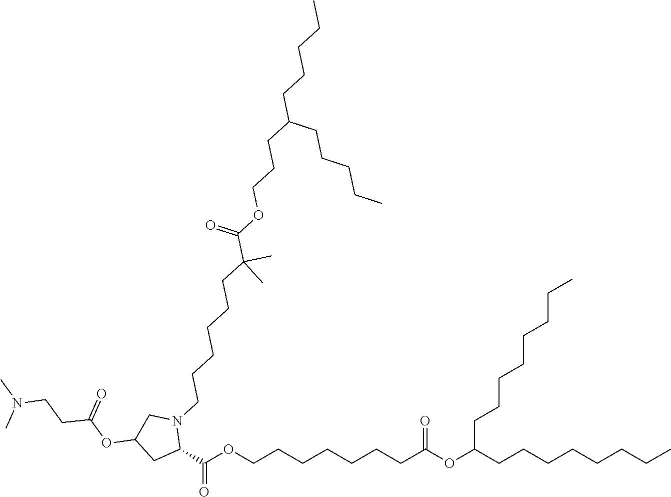 [SMILES: CCCCCCCCC(CCCCCCCC)OC(=O)CCCCCCCOC(=O)[C@@H]1CC(OC(=O)CCN(C)C)CN1CCCCCCC(C)(C)C(=O)OCCCC(CCCCC)CCCCC] |
| 2366 | 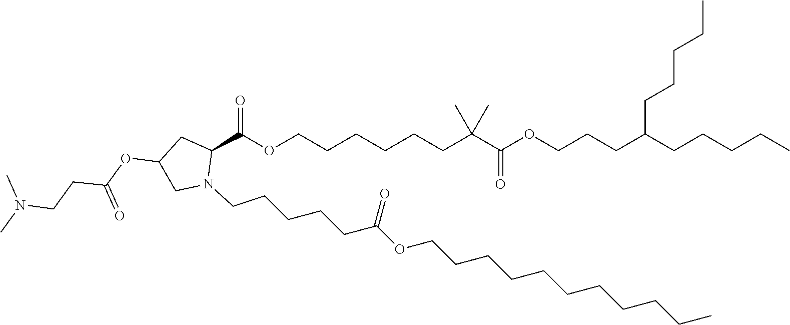 [SMILES: CCCCCCCCCCCOC(=O)CCCCCN1CC(OC(=O)CCN(C)C)C[C@H]1C(=O)OCCCCCCC(C)(C)C(=O)OCCCC(CCCCC)CCCCC] |
| 2367 | 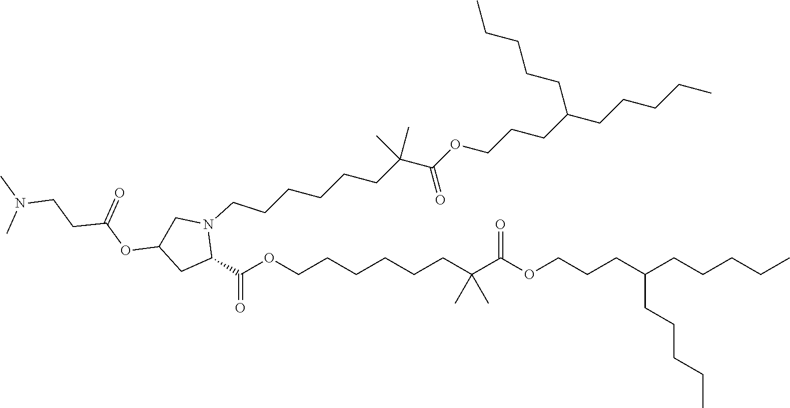 [SMILES: CCCCCC(CCCCC)CCCOC(=O)C(C)(C)CCCCCCOC(=O)[C@@H]1CC(OC(=O)CCN(C)C)CN1CCCCCCC(C)(C)C(=O)OCCCC(CCCCC)CCCCC] |
| 2368 | 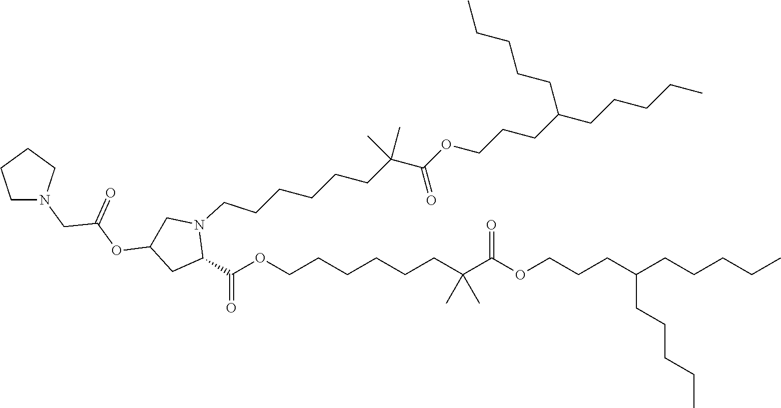 [SMILES: CCCCCC(CCCCC)CCCOC(=O)C(C)(C)CCCCCCOC(=O)[C@@H]1CC(OC(=O)CN2CCCC2)CN1CCCCCCC(C)(C)C(=O)OCCCC(CCCCC)CCCCC] |
| 2369 | 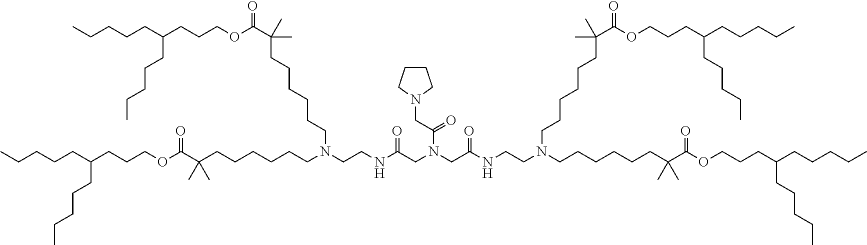 [SMILES: CCCCCC(CCCCC)CCCOC(=O)C(C)(C)CCCCCCN(CCCCCCC(C)(C)C(=O)OCCCC(CCCCC)CCCCC)CCNC(=O)CN(CC(=O)NCCN(CCCCCCC(C)(C)C(=O)OCCCC(CCCCC)CCCCC)CCCCCCC(C)(C)C(=O)OCCCC(CCCCC)CCCCC)C(=O)CN1CCCC1] |
| 2383 | 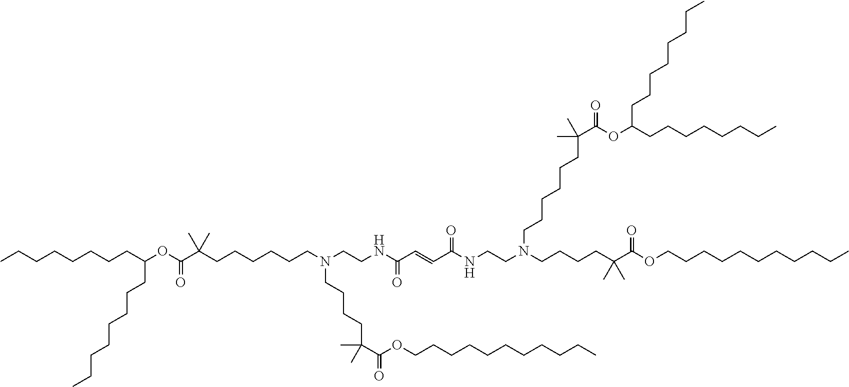 [SMILES: CCCCCCCCCCCOC(=O)C(C)(C)CCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] |
| 2392 | 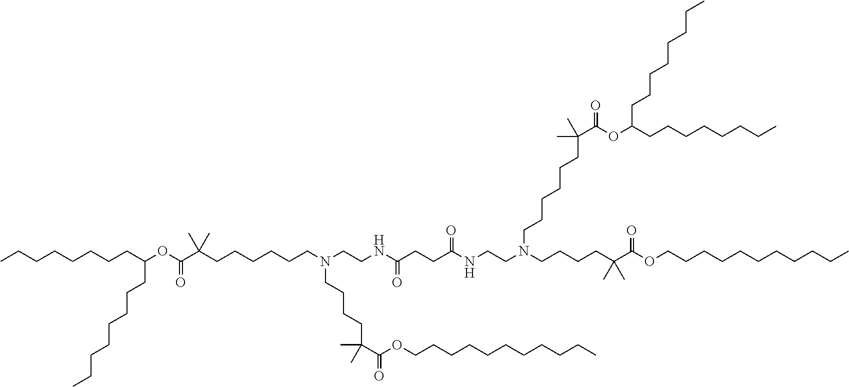 [SMILES: CCCCCCCCCCCOC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] |
| 2398 | 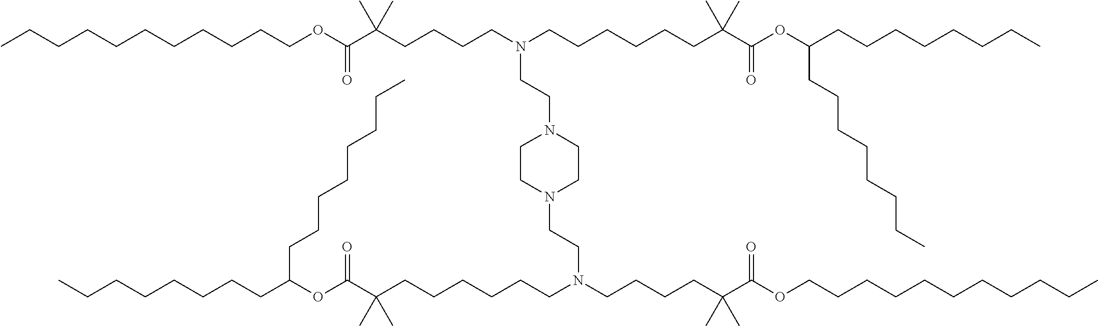 |
| 2424 | 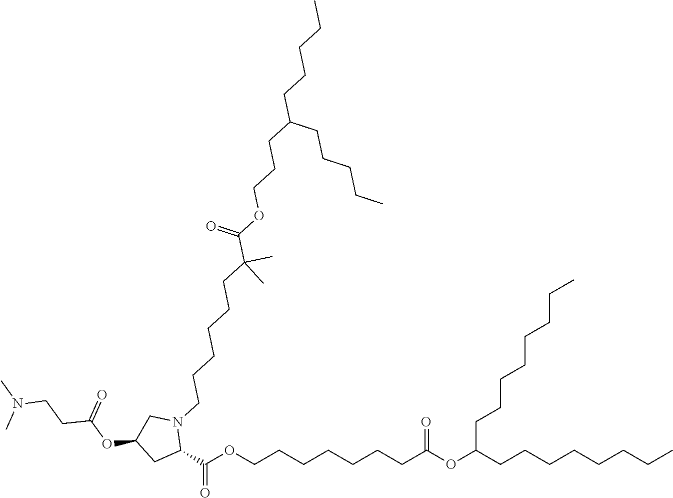 [SMILES: CCCCCCCCC(CCCCCCCC)OC(=O)CCCCCCCOC(=O)[C@@H]1C[C@@H](OC(=O)CCN(C)C)CN1CCCCCCC(C)(C)C(=O)OCCCC(CCCCC)CCCCC] |
| 2425 | 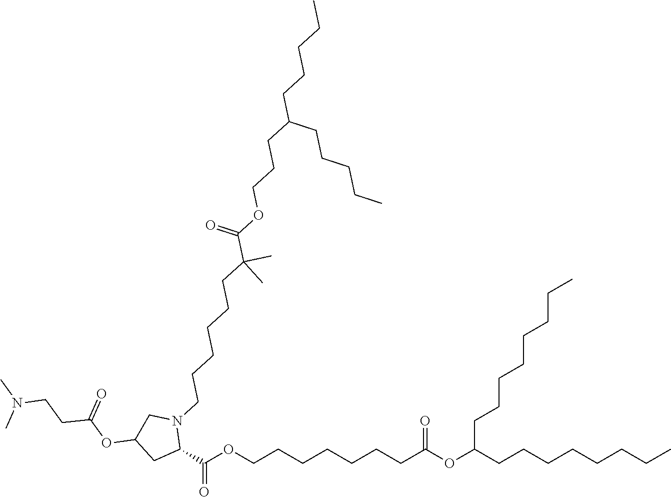 [SMILES: CCCCCCCCC(CCCCCCCC)OC(=O)CCCCCCCOC(=O)[C@@H]1CC(OC(=O)CCN(C)C)CN1CCCCCCC(C)(C)C(=O)OCCCC(CCCCC)CCCCC] |
| 2432 | 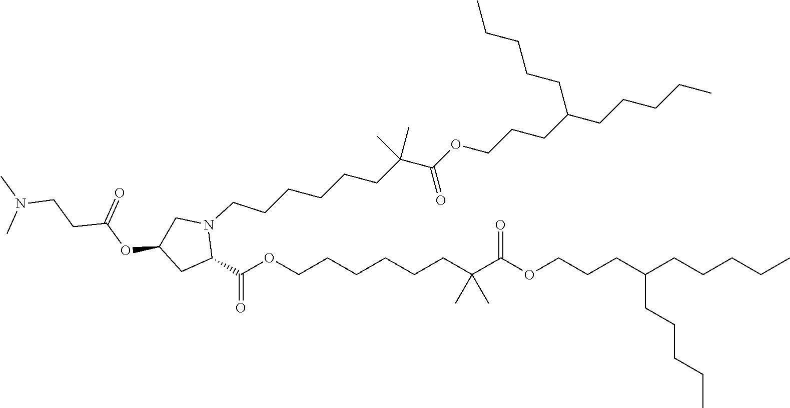 [SMILES: CCCCCC(CCCCC)CCCOC(=O)C(C)(C)CCCCCCOC(=O)[C@@H]1C[C@@H](OC(=O)CCN(C)C)CN1CCCCCCC(C)(C)C(=O)OCCCC(CCCCC)CCCCC] |
| 2433 | 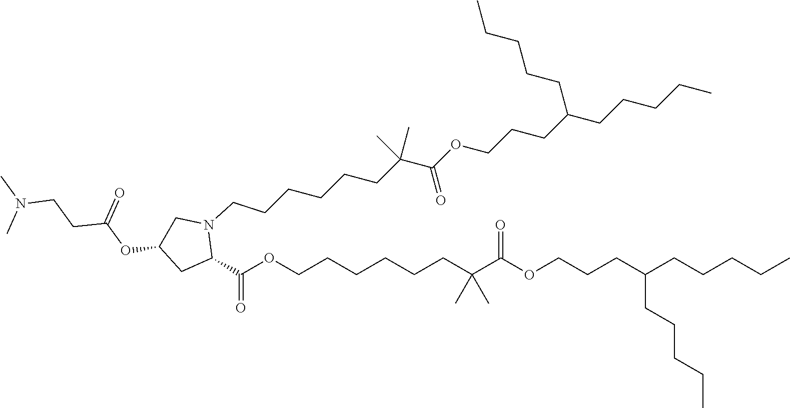 [SMILES: CCCCCC(CCCCC)CCCOC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] |
| 2441 | 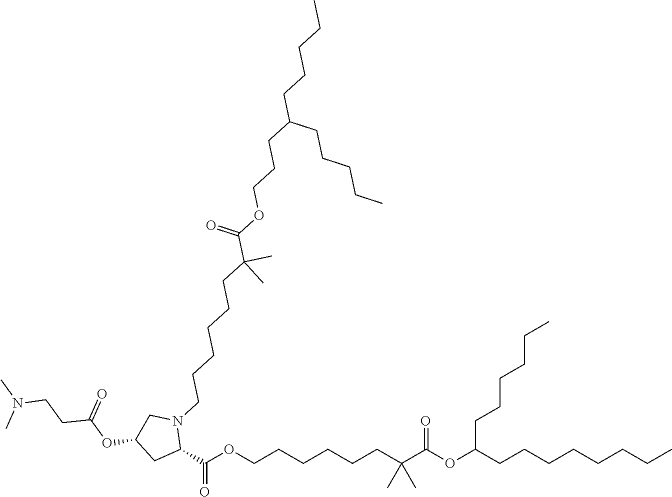 [SMILES: CCCCCCCCC(CCCCCC)OC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] |
| 2442 | 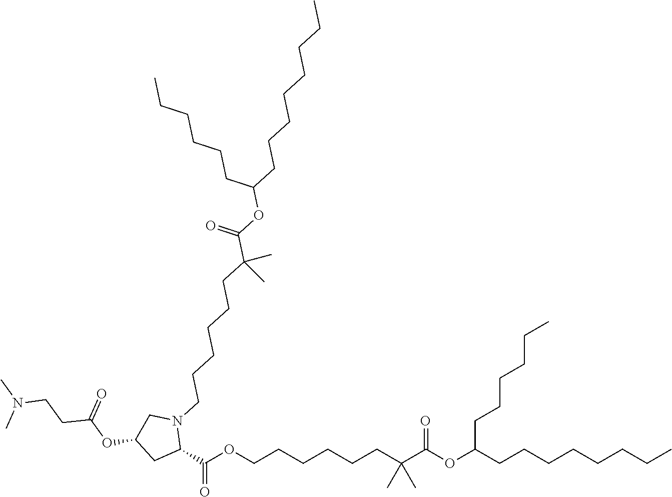 [SMILES: CCCCCCCCC(CCCCCC)OC(=O)C(C)(C)CCCCCCOC(=O)[C@@H]1C[C@H](OC(=O)CCN(C)C)CN1CCCCCCC(C)(C)C(=O)OC(CCCCCC)CCCCCCCC] |
| 2443 | 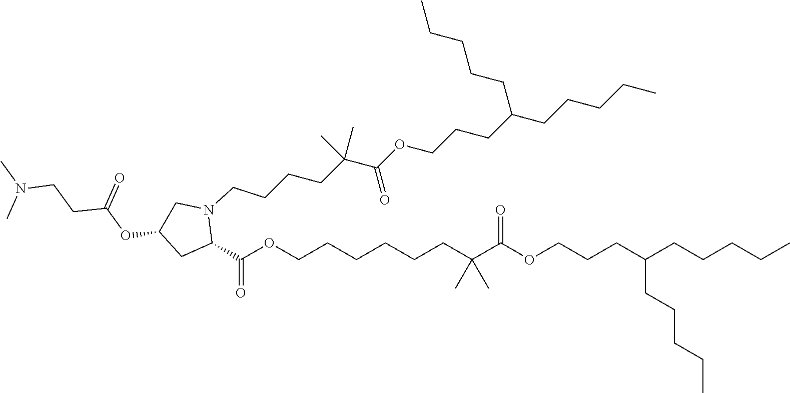 [SMILES: CCCCCC(CCCCC)CCCOC(=O)C(C)(C)CCCCCCOC(=O)[C@@H]1C[C@H](OC(=O)CCN(C)C)CN1CCCCC(C)(C)C(=O)OCCCC(CCCCC)CCCCC] |
| 2454 | 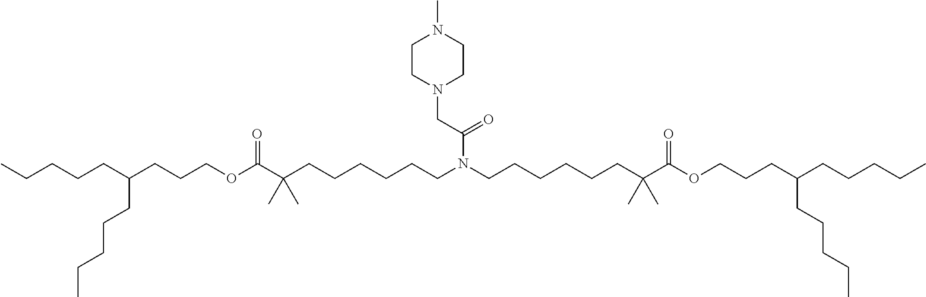 [SMILES: CCCCCC(CCCCC)CCCOC(=O)C(C)(C)CCCCCCN(CCCCCCC(C)(C)C(=O)OCCCC(CCCCC)CCCCC)C(=O)CN1CCN(C)CC1] |
| 2481 | 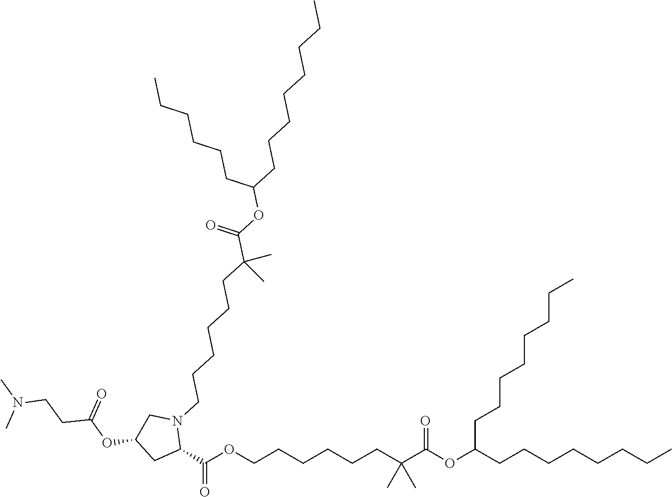 [SMILES: CCCCCCCCC(CCCCCC)OC(=O)C(C)(C)CCCCCCN1C[C@@H](OC(=O)CCN(C)C)C[C@H]1C(=O)OCCCCCCC(C)(C)C(=O)OC(CCCCCCCC)CCCCCCCC] |
| 2482 | 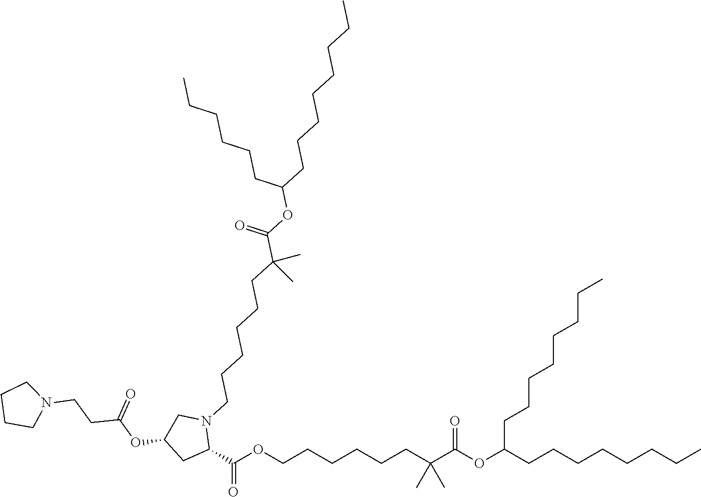 [SMILES: CCCCCCCCC(CCCCCC)OC(=O)C(C)(C)CCCCCCN1C[C@@H](OC(=O)CCN2CCCC2)C[C@H]1C(=O)OCCCCCCC(C)(C)C(=O)OC(CCCCCCCC)CCCCCCCC] |
| 2483 | 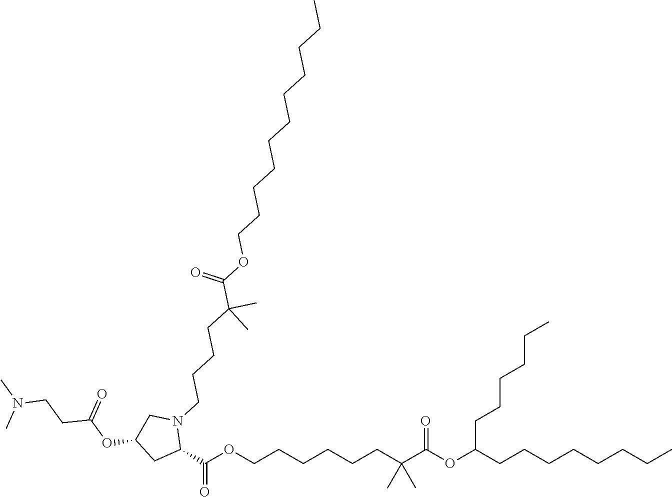 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCC)CCCCCCCC] |
| 2486 | 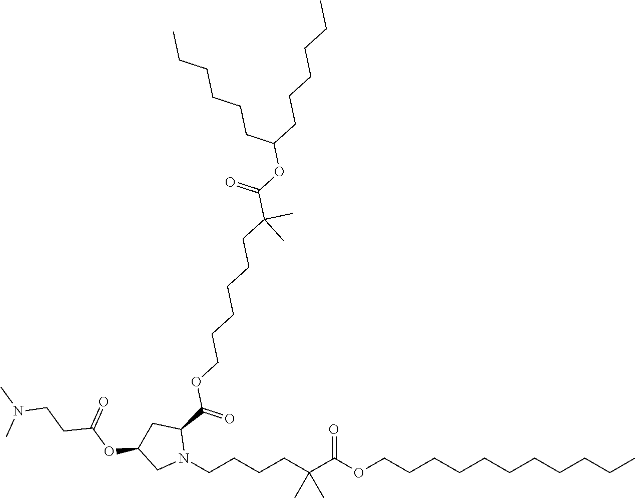 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCC)CCCCCC] |
| 2487 | 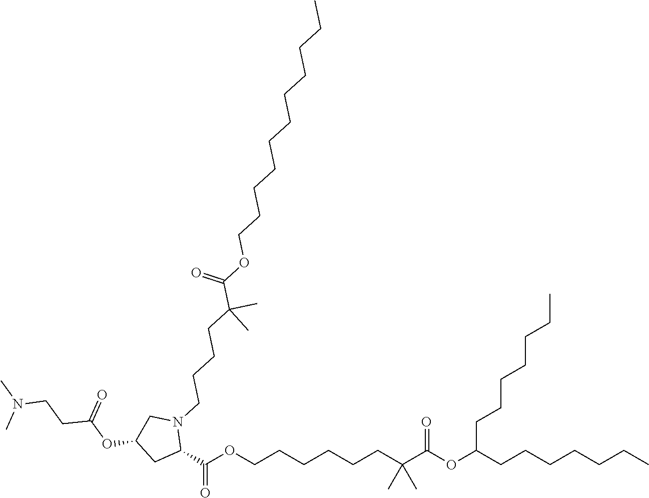 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCCC)CCCCCCC] |
| 2488 | 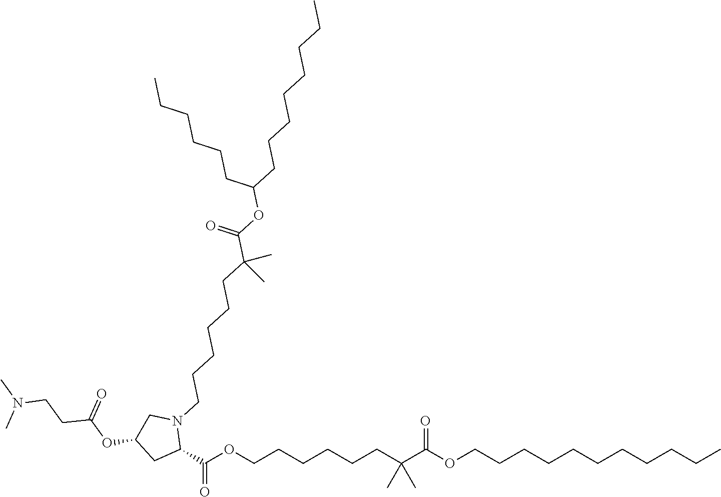 [SMILES: CCCCCCCCCCCOC(=O)C(C)(C)CCCCCCOC(=O)[C@@H]1C[C@H](OC(=O)CCN(C)C)CN1CCCCCCC(C)(C)C(=O)OC(CCCCCC)CCCCCCCC] |
| 2498 | 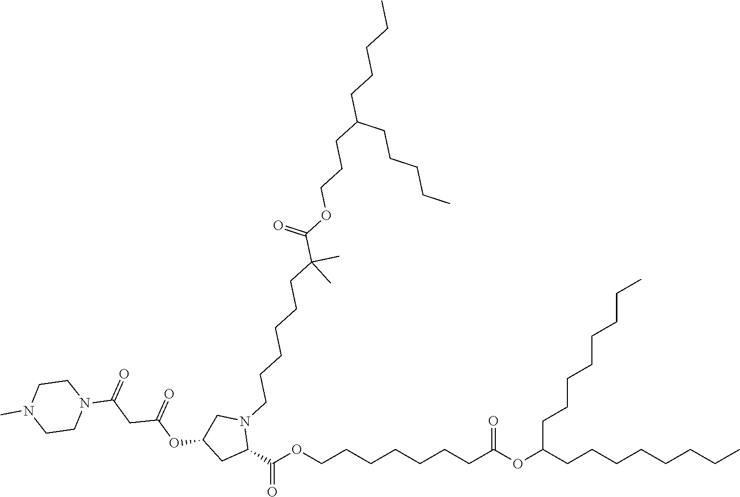 [SMILES: CCCCCCCCC(CCCCCCCC)OC(=O)CCCCCCCOC(=O)[C@@H]1C[C@H](OC(=O)CC(=O)N2CCN(C)CC2)CN1CCCCCCC(C)(C)C(=O)OCCCC(CCCCC)CCCCC] |
| 2500 | 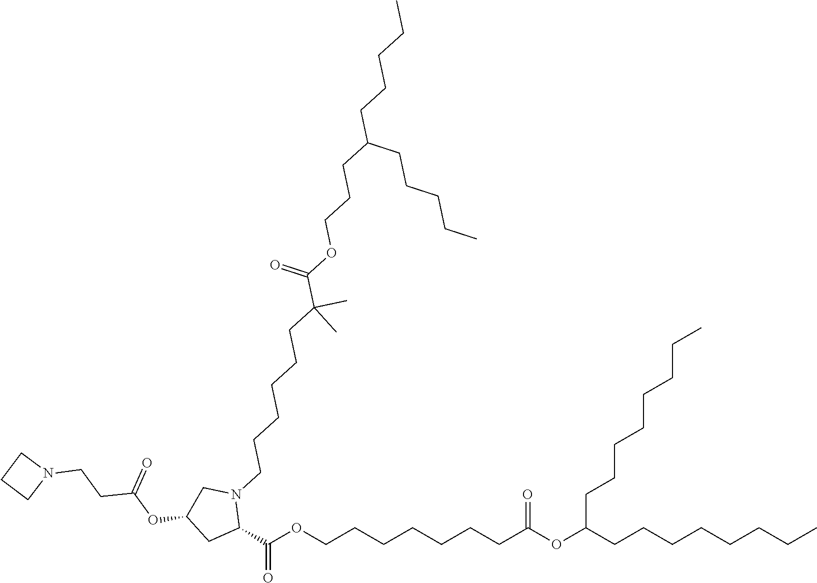 [SMILES: CCCCCCCCC(CCCCCCCC)OC(=O)CCCCCCCOC(=O)[C@@H]1C[C@H](OC(=O)CCN2CCC2)CN1CCCCCCC(C)(C)C(=O)OCCCC(CCCCC)CCCCC] |
| 2501 | 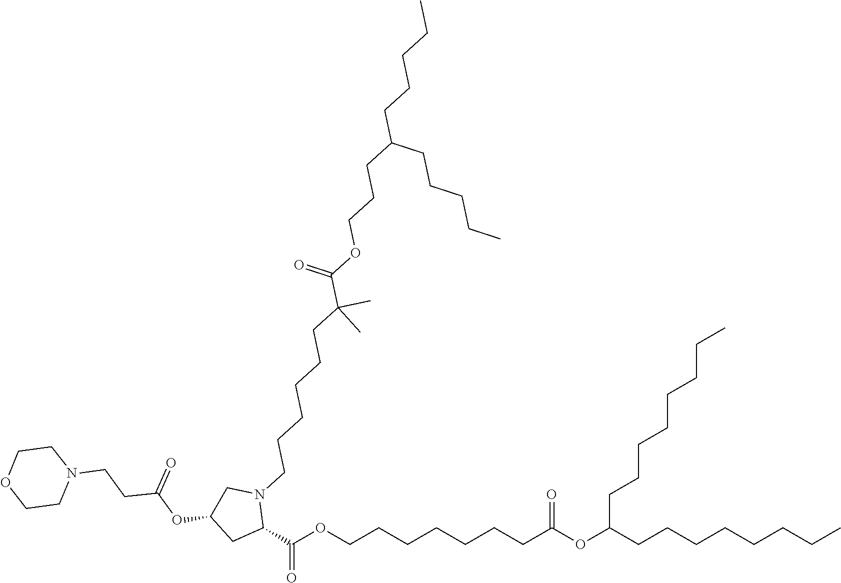 [SMILES: CCCCCCCCC(CCCCCCCC)OC(=O)CCCCCCCOC(=O)[C@@H]1C[C@H](OC(=O)CCN2CCOCC2)CN1CCCCCCC(C)(C)C(=O)OCCCC(CCCCC)CCCCC] |
| 2505 | 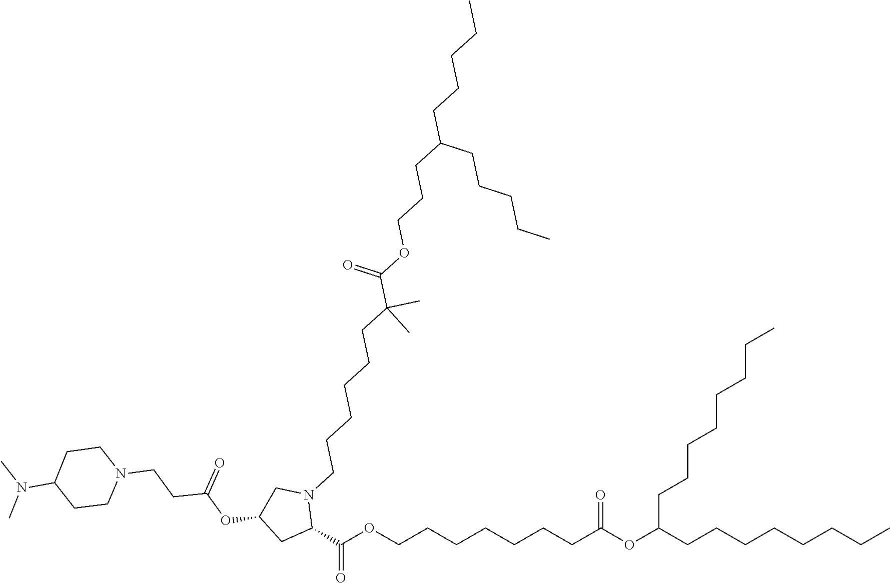 [SMILES: CCCCCCCCC(CCCCCCCC)OC(=O)CCCCCCCOC(=O)[C@@H]1C[C@H](OC(=O)CCN2CCC(N(C)C)CC2)CN1CCCCCCC(C)(C)C(=O)OCCCC(CCCCC)CCCCC] |
| 2504 | 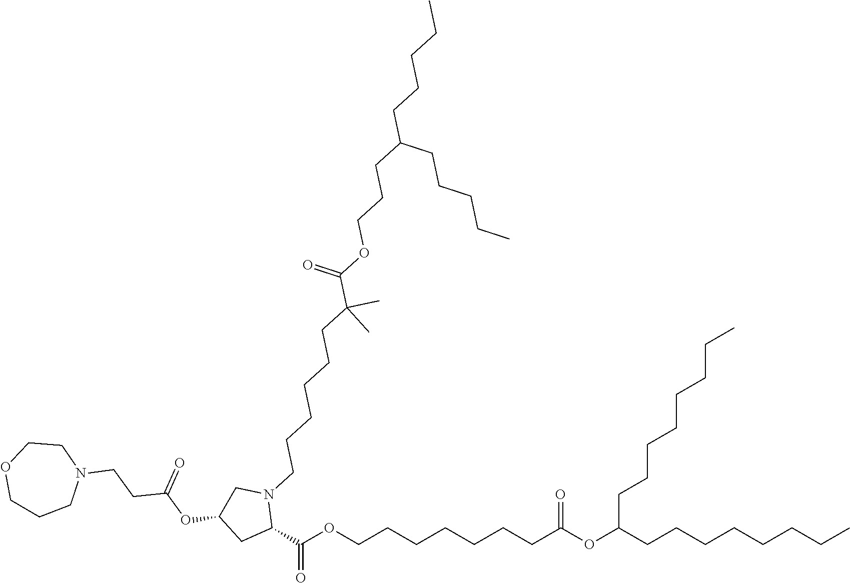 [SMILES: CCCCCCCCC(CCCCCCCC)OC(=O)CCCCCCCOC(=O)[C@@H]1C[C@H](OC(=O)CCN2CCCOCC2)CN1CCCCCCC(C)(C)C(=O)OCCCC(CCCCC)CCCCC] |
| 2503 | 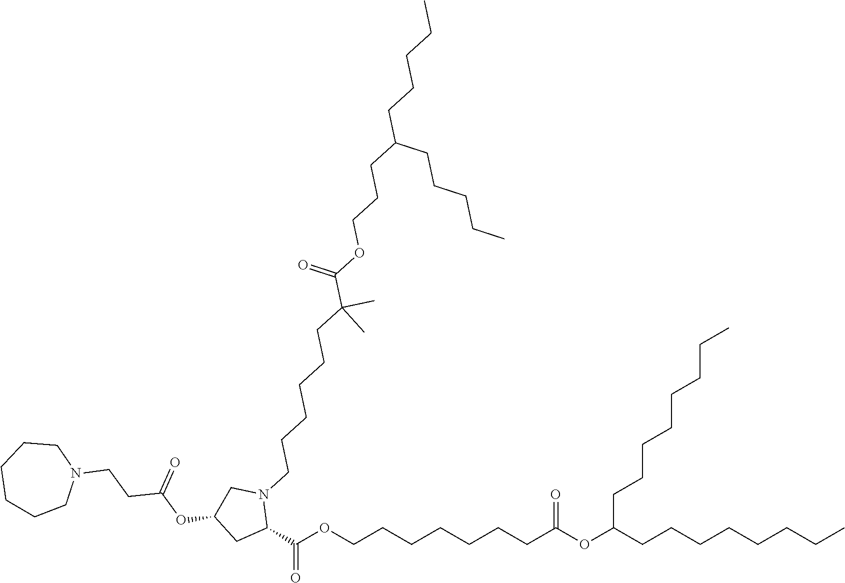 [SMILES: CCCCCCCCC(CCCCCCCC)OC(=O)CCCCCCCOC(=O)[C@@H]1C[C@H](OC(=O)CCN2CCCCCC2)CN1CCCCCCC(C)(C)C(=O)OCCCC(CCCCC)CCCCC] |
| 2502 | 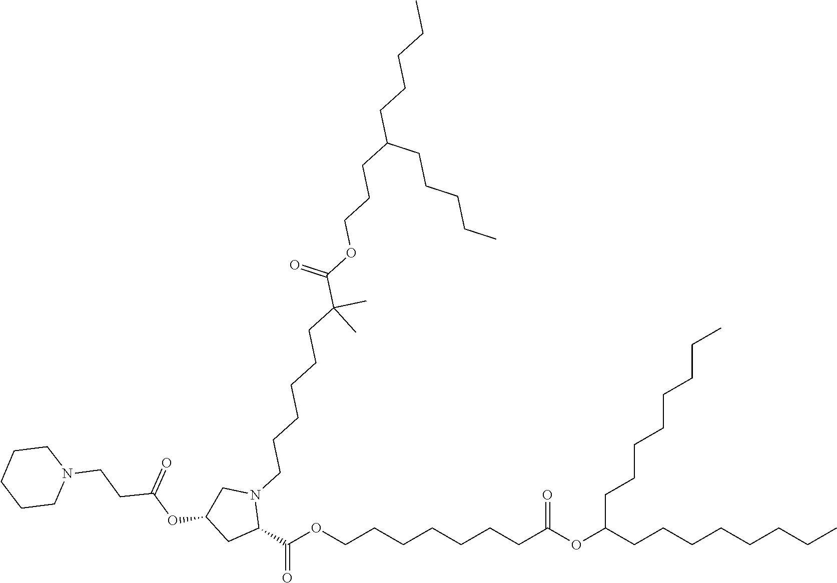 [SMILES: CCCCCCCCC(CCCCCCCC)OC(=O)CCCCCCCOC(=O)[C@@H]1C[C@H](OC(=O)CCN2CCCCC2)CN1CCCCCCC(C)(C)C(=O)OCCCC(CCCCC)CCCCC] |
| 2508 | 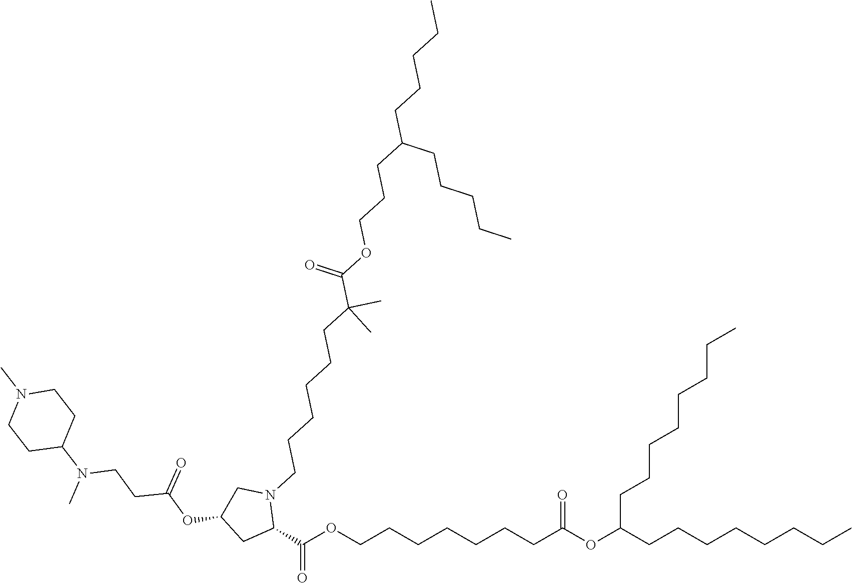 [SMILES: CCCCCCCCC(CCCCCCCC)OC(=O)CCCCCCCOC(=O)[C@@H]1C[C@H](OC(=O)CCN(C)C2CCN(C)CC2)CN1CCCCCCC(C)(C)C(=O)OCCCC(CCCCC)CCCCC] |
| 2510 | 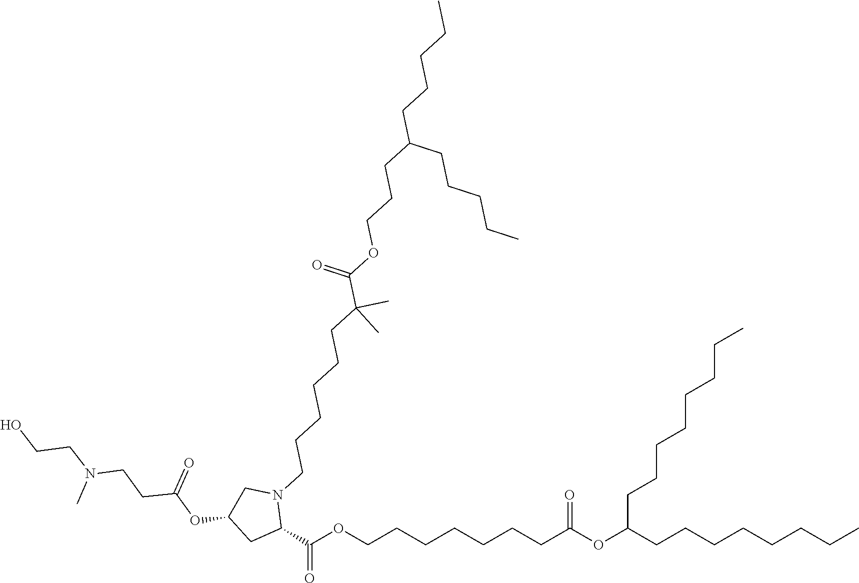 [SMILES: CCCCCCCCC(CCCCCCCC)OC(=O)CCCCCCCOC(=O)[C@@H]1C[C@H](OC(=O)CCN(C)CCO)CN1CCCCCCC(C)(C)C(=O)OCCCC(CCCCC)CCCCC] |
| 2511 | 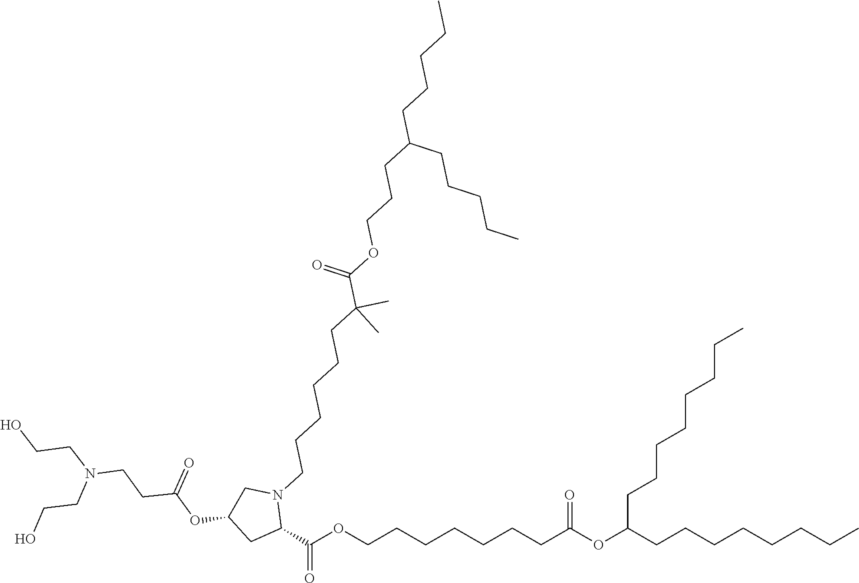 [SMILES: CCCCCCCCC(CCCCCCCC)OC(=O)CCCCCCCOC(=O)[C@@H]1C[C@H](OC(=O)CCN(CCO)CCO)CN1CCCCCCC(C)(C)C(=O)OCCCC(CCCCC)CCCCC] |
| 2509 | 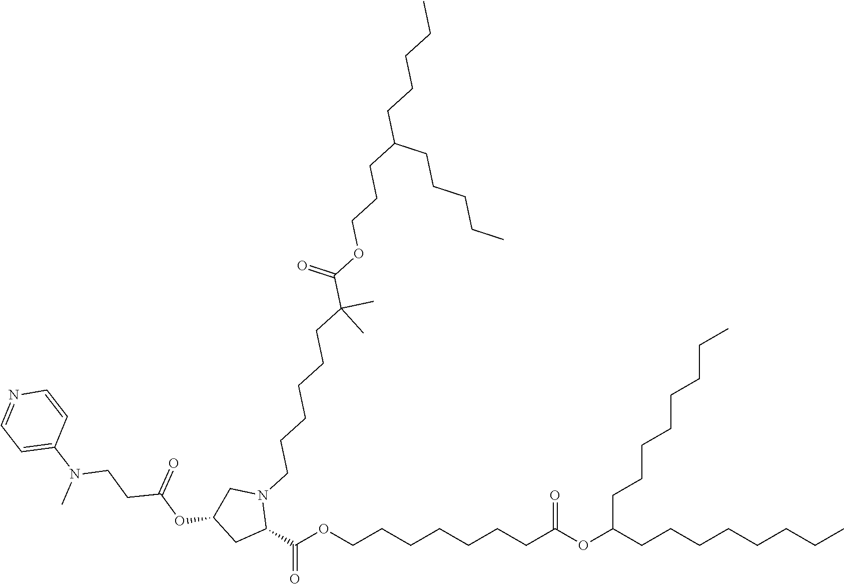 [SMILES: CCCCCCCCC(CCCCCCCC)OC(=O)CCCCCCCOC(=O)[C@@H]1C[C@H](OC(=O)CCN(C)c2ccncc2)CN1CCCCCCC(C)(C)C(=O)OCCCC(CCCCC)CCCCC] |
| 2507 | 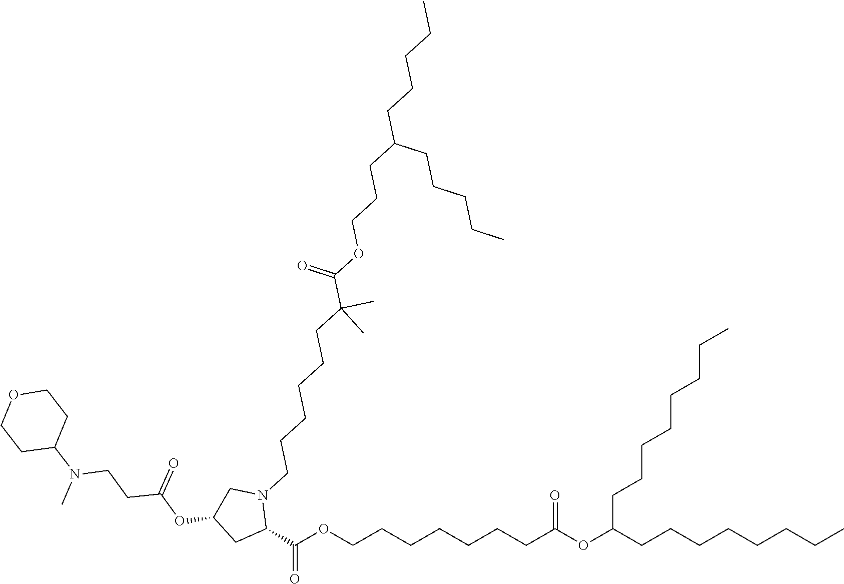 [SMILES: CCCCCCCCC(CCCCCCCC)OC(=O)CCCCCCCOC(=O)[C@@H]1C[C@H](OC(=O)CCN(C)C2CCOCC2)CN1CCCCCCC(C)(C)C(=O)OCCCC(CCCCC)CCCCC] |
| 2506 | 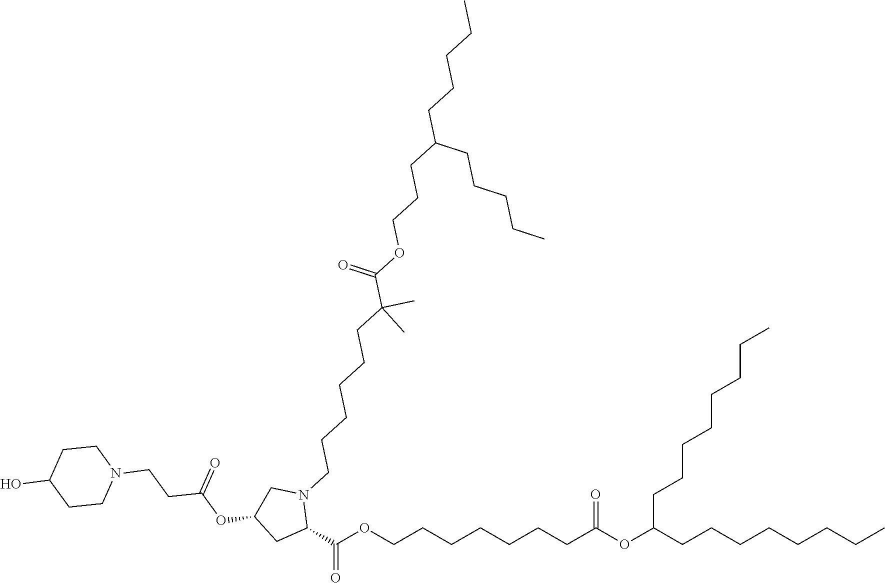 [SMILES: CCCCCCCCC(CCCCCCCC)OC(=O)CCCCCCCOC(=O)[C@@H]1C[C@H](OC(=O)CCN2CCC(O)CC2)CN1CCCCCCC(C)(C)C(=O)OCCCC(CCCCC)CCCCC] |
| 2499 | 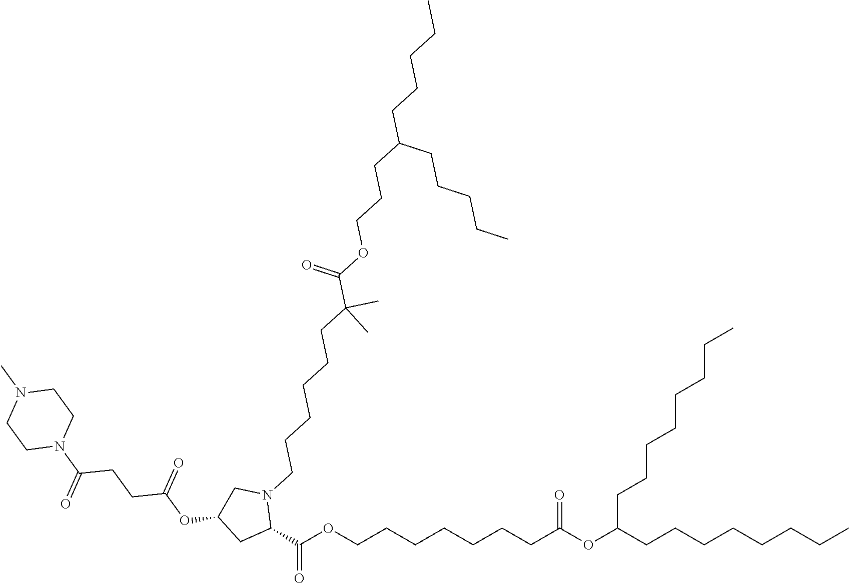 [SMILES: CCCCCCCCC(CCCCCCCC)OC(=O)CCCCCCCOC(=O)[C@@H]1C[C@H](OC(=O)CCC(=O)N2CCN(C)CC2)CN1CCCCCCC(C)(C)C(=O)OCCCC(CCCCC)CCCCC] |
| 2485 | 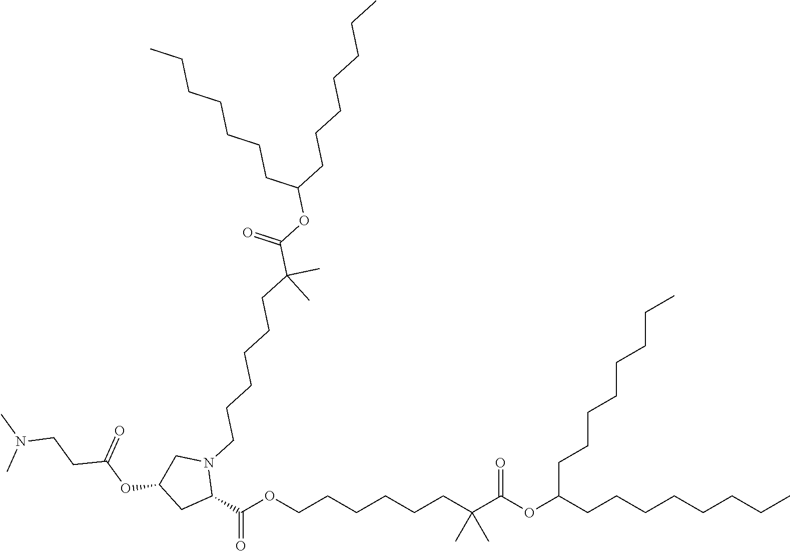 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OC(CCCCCCC)CCCCCCC] |
| 2484 | 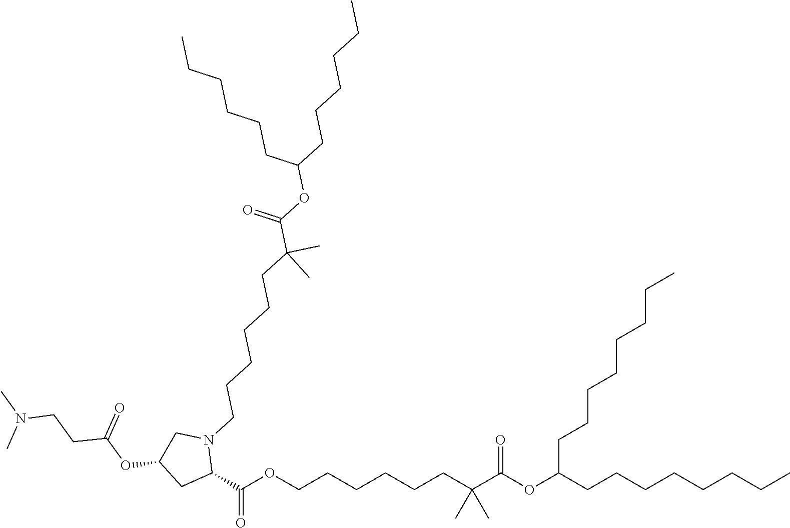 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OC(CCCCCC)CCCCCC] |
| 2535 | 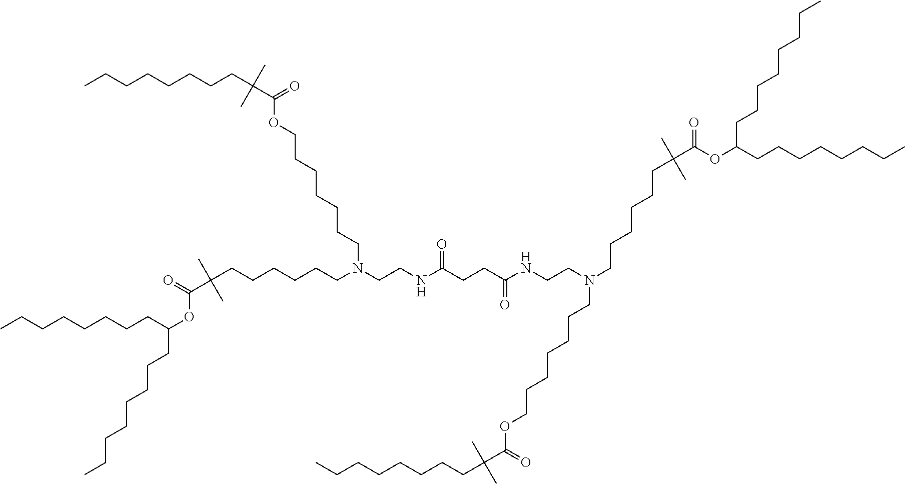 [SMILES: CCCCCCCCC(CCCCCCCC)OC(=O)C(C)(C)CCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] |
| 2536 | 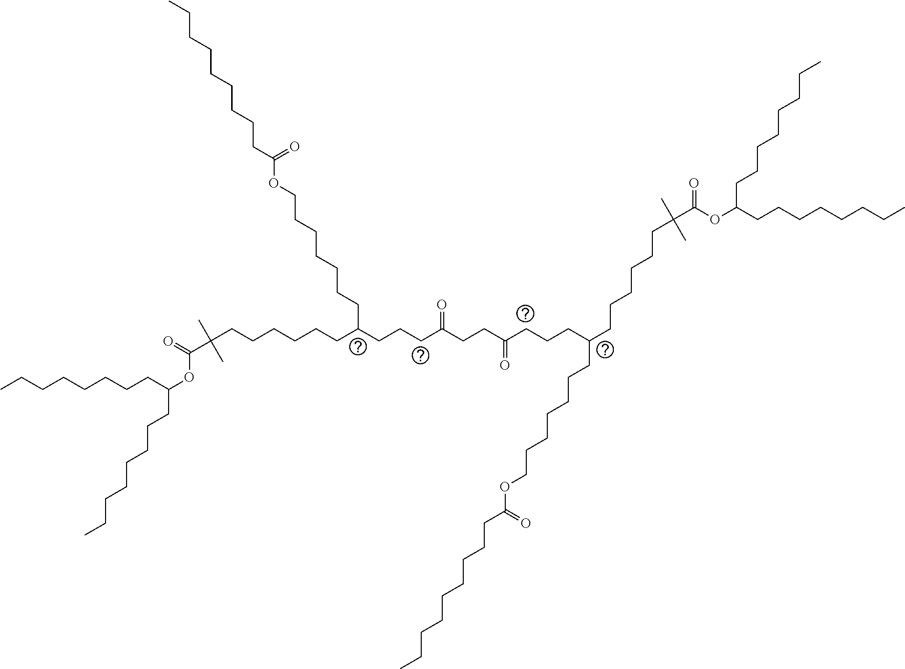 [SMILES: CCCCCCCCCC(=O)OCCCCCCCC(CCCCCCC(C)(C)C(=O)OC(CCCCCCCC)CCCCCCCC)CCCC(=O)CCC(=O)CCCC(CCCCCCCOC(=O)CCCCCCCCC)CCCCCCC(C)(C)C(=O)OC(CCCCCCCC)CCCCCCCC] |
| 2537 | 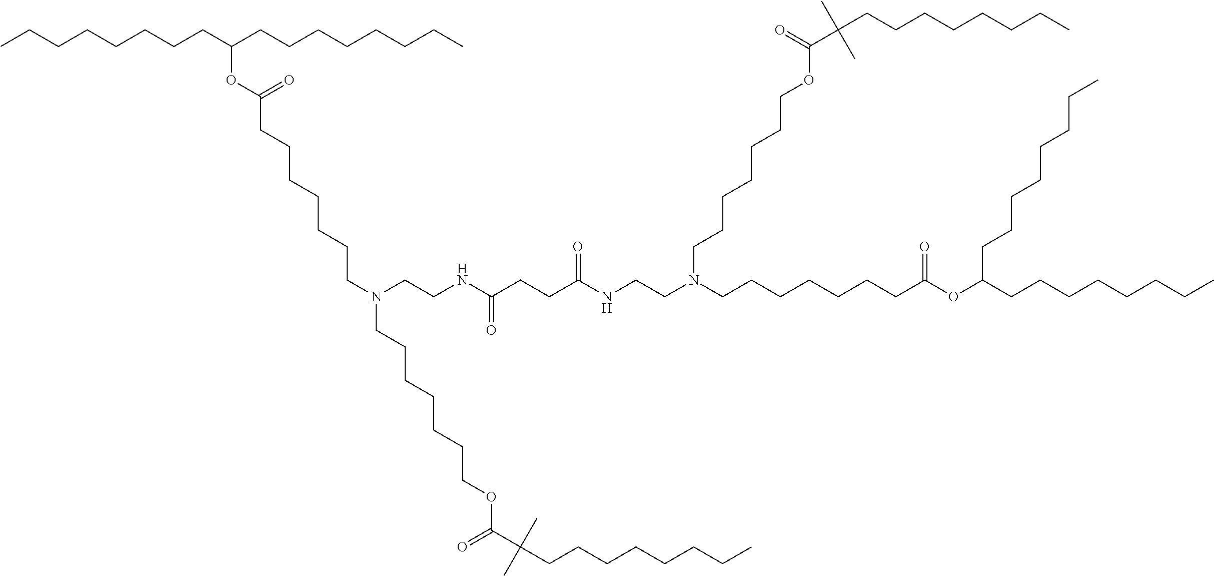 [SMILES: CCCCCCCCC(CCCCCCCC)OC(=O)CCCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] |
| 2544 | 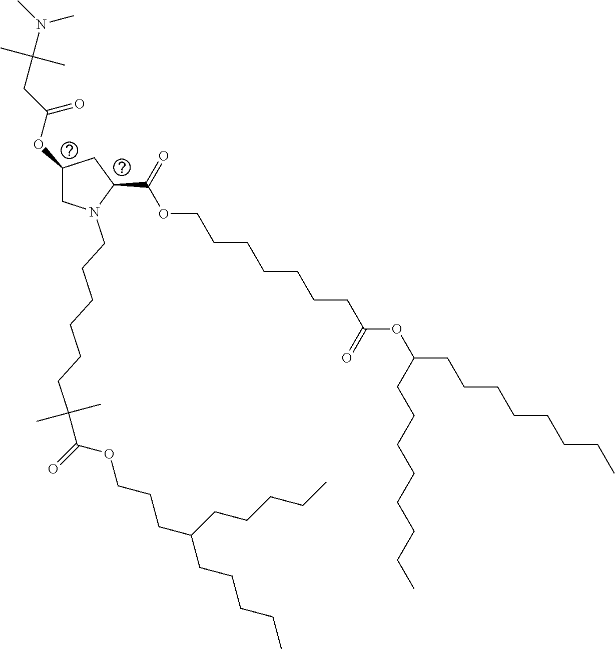 [SMILES: CCCCCCCCC(CCCCCCCC)OC(=O)CCCCCCCOC(=O)[C@@H]1C[C@H](OC(=O)CC(C)(C)N(C)C)CN1CCCCCCC(C)(C)C(=O)OCCCC(CCCCC)CCCCC] |
| 2545 | 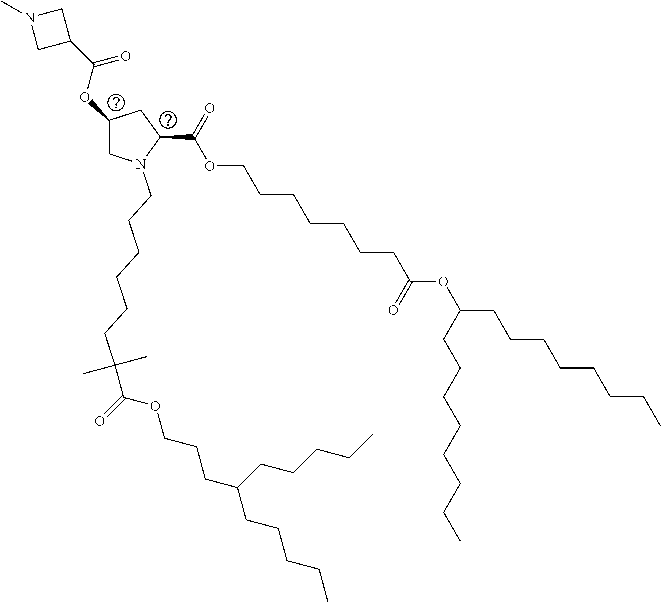 [SMILES: CCCCCCCCC(CCCCCCCC)OC(=O)CCCCCCCOC(=O)[C@@H]1C[C@H](OC(=O)C2CN(C)C2)CN1CCCCCCC(C)(C)C(=O)OCCCC(CCCCC)CCCCC] |
| 2546 | 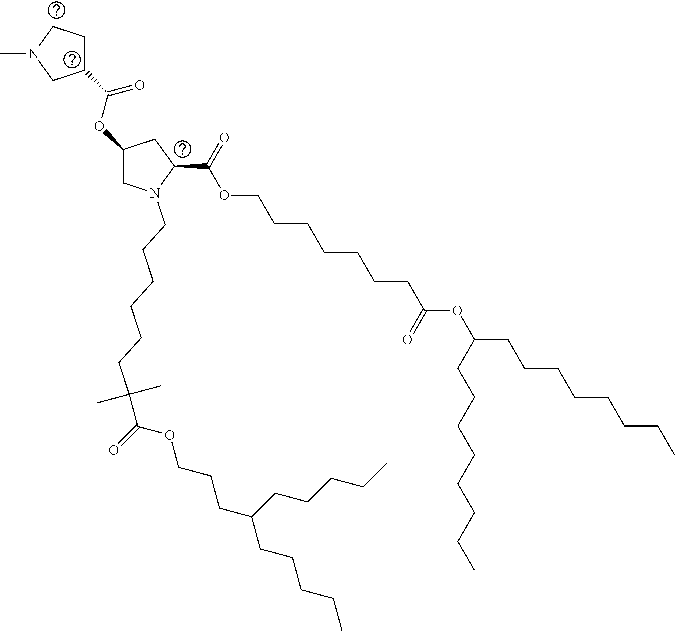 |
| 2547 | 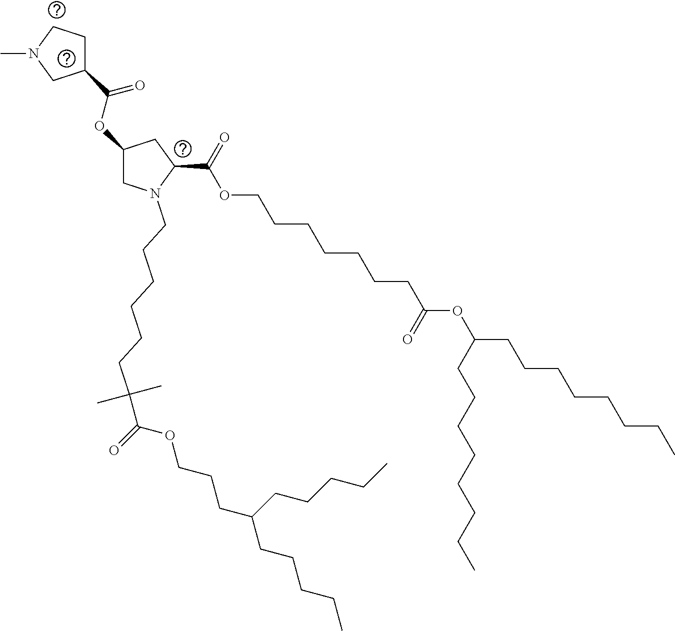 [SMILES: CCCCCCCCC(CCCCCCCC)OC(=O)CCCCCCCOC(=O)[C@@H]1C[C@H](OC(=O)[C@@H]2CCN(C)C2)CN1CCCCCCC(C)(C)C(=O)OCCCC(CCCCC)CCCCC] |
| 2551 | 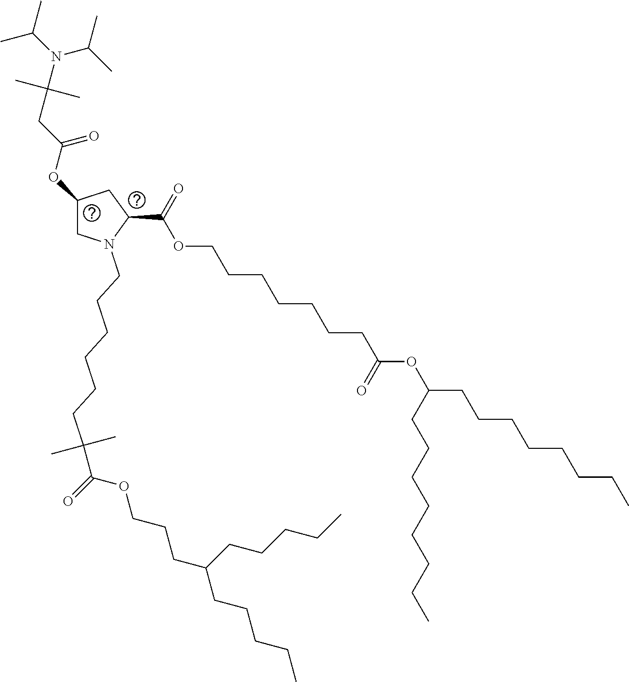 [SMILES: CCCCCCCCC(CCCCCCCC)OC(=O)CCCCCCCOC(=O)[C@@H]1C[C@H](OC(=O)CC(C)(C)N(C(C)C)C(C)C)CN1CCCCCCC(C)(C)C(=O)OCCCC(CCCCC)CCCCC] |
| 2552 | 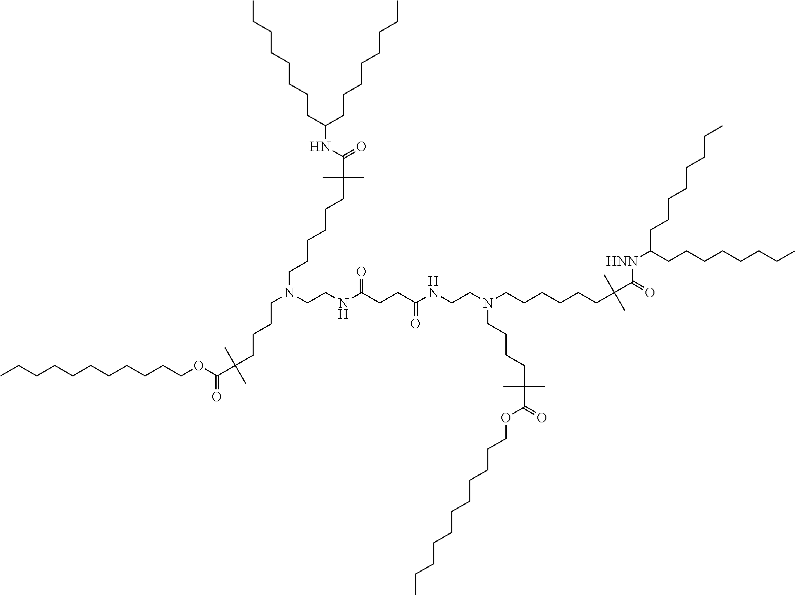 |
| 2553 | 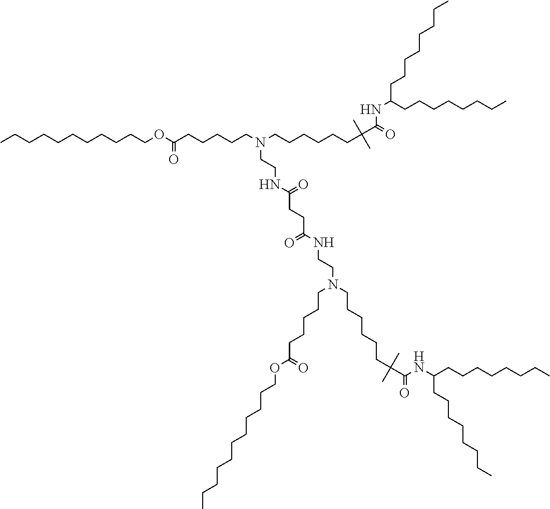 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OCCCCCCCCCCC] |
| 2554 | 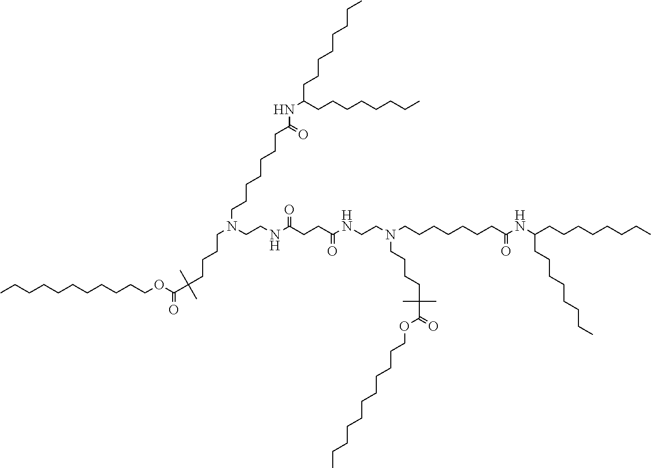 [SMILES: CCCCCCCCCCCOC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] |
| 2555 | 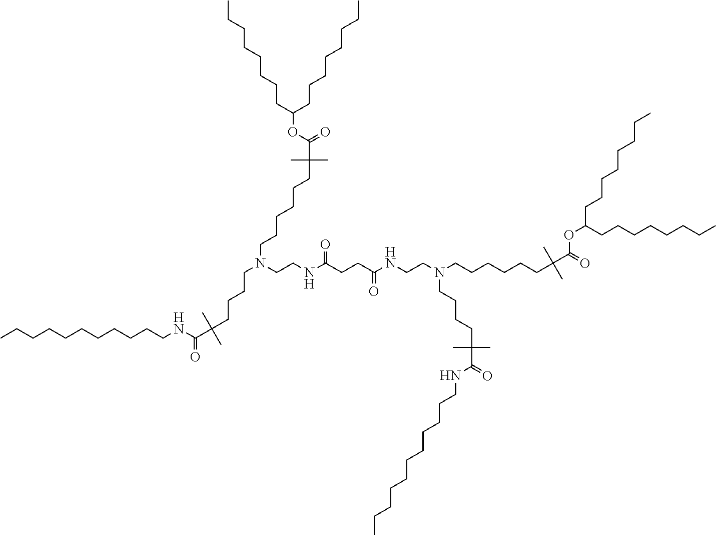 [SMILES: CCCCCCCCCCCNC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)NCCCCCCCCCCC] |
| 2556 | 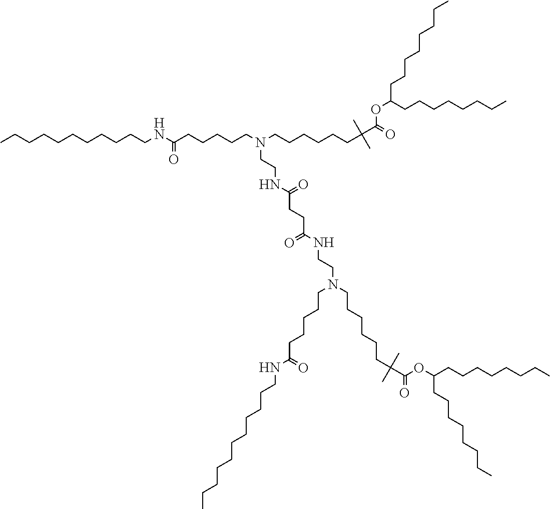 [SMILES: CCCCCCCCCCCNC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)NCCCCCCCCCCC] |
| 2557 | 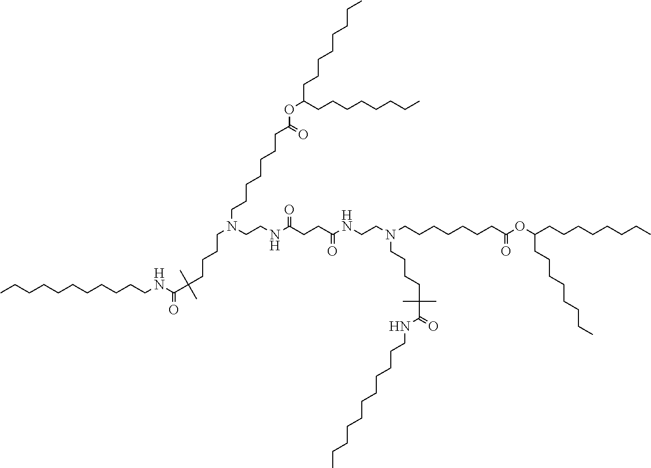 [SMILES: CCCCCCCCCCCNC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)NCCCCCCCCCCC] |
| 2559 | 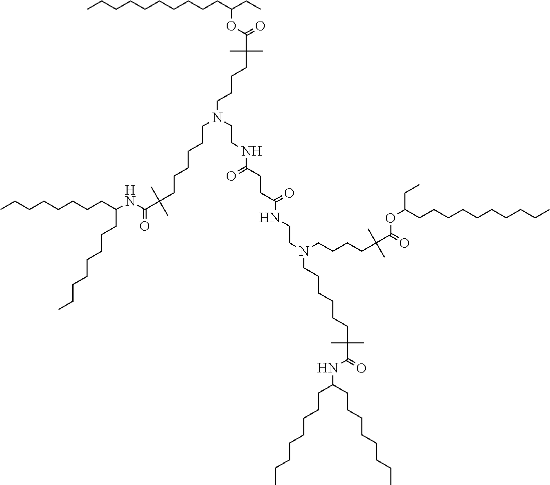 [SMILES: CCCCCCCCCCC(CC)OC(=O)C(C)(C)CCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OC(CC)CCCCCCCCCC] |
| 2560 | 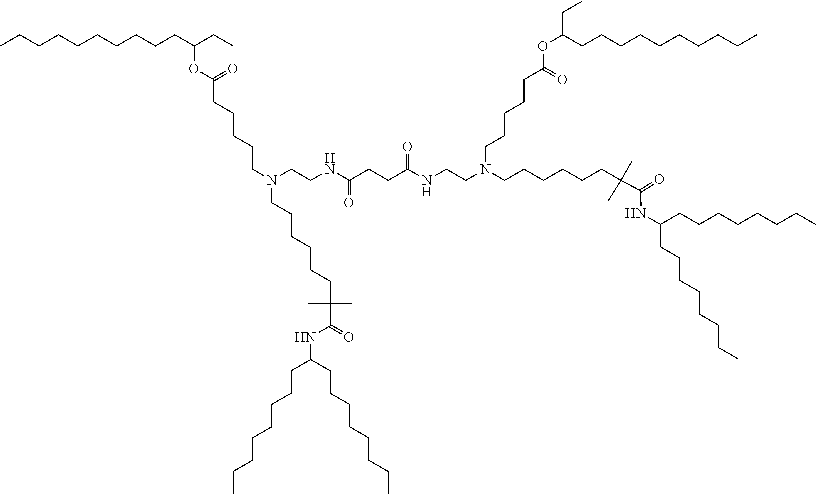 [SMILES: CCCCCCCCCCC(CC)OC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OC(CC)CCCCCCCCCC] |
| 2561 | 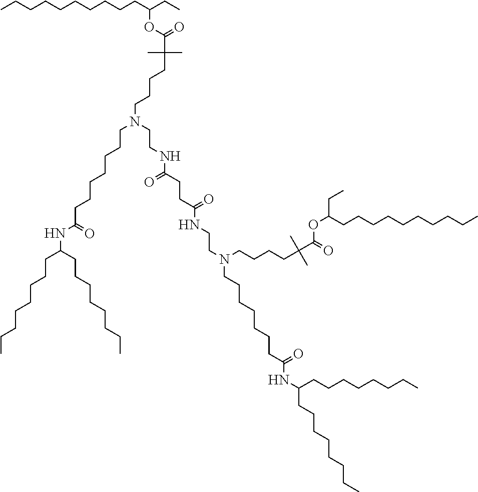 [SMILES: CCCCCCCCCCC(CC)OC(=O)C(C)(C)CCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OC(CC)CCCCCCCCCC] |
| 2563 | 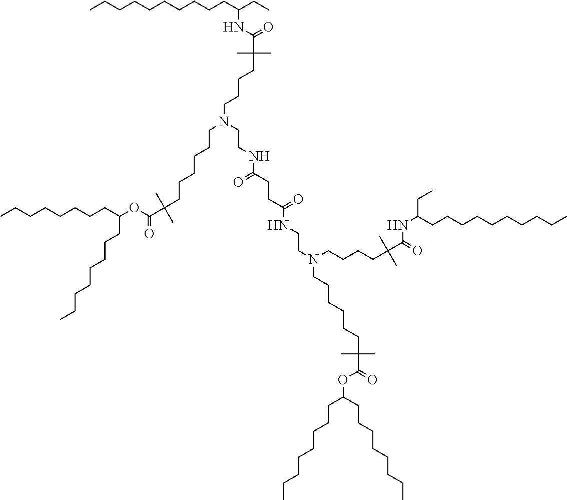 [SMILES: CCCCCCCCCCC(CC)NC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)NC(CC)CCCCCCCCCC] |
| 2564 | 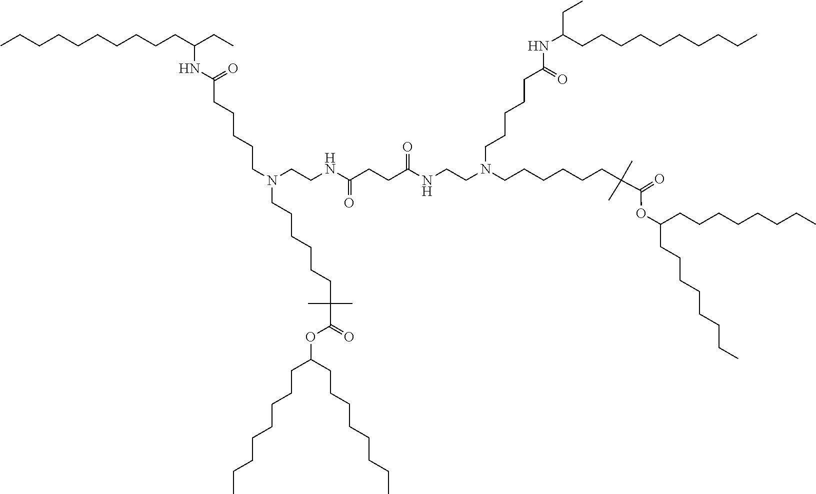 [SMILES: CCCCCCCCCCC(CC)NC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)NC(CC)CCCCCCCCCC] |
| 2565 | 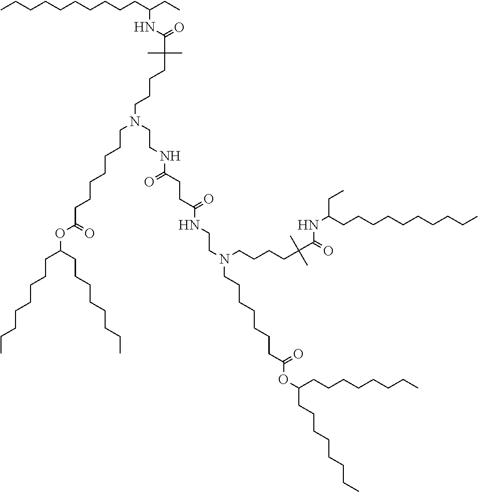 [SMILES: CCCCCCCCCCC(CC)NC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)NC(CC)CCCCCCCCCC] |
| 2566 | 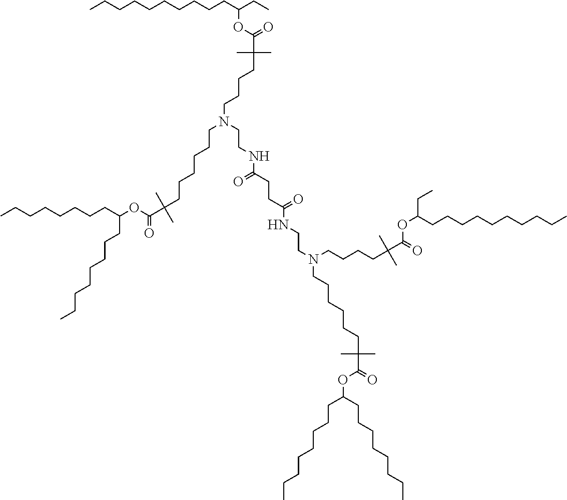 [SMILES: CCCCCCCCCCC(CC)OC(=O)C(C)(C)CCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] |
| 2567 | 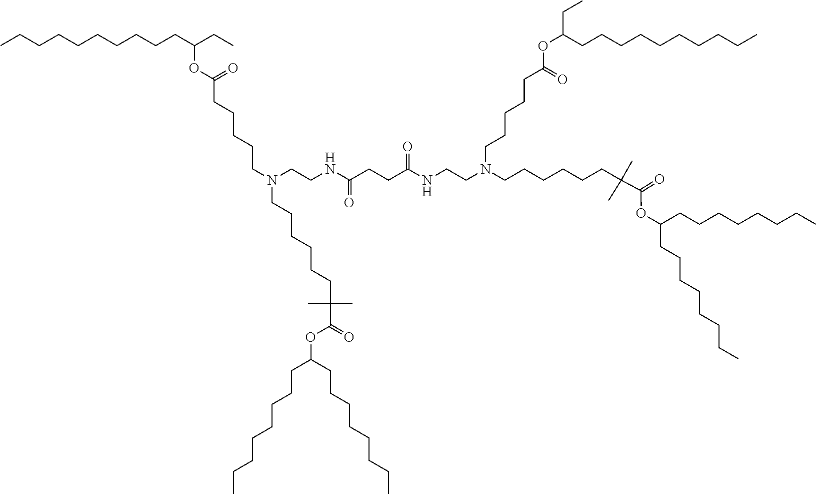 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OC(CC)CCCCCCCCCC] |
| 2568 | 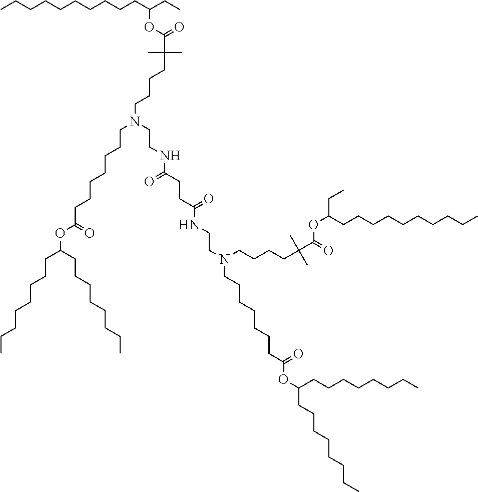 [SMILES: CCCCCCCCCCC(CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] |
| 2569 | 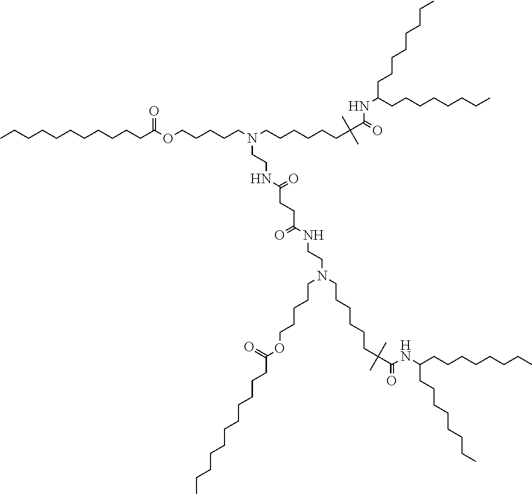 [SMILES: CCCCCCCCCCCC(=O)OCCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCCOC(=O)CCCCCCCCCCC] |
| 2570 | 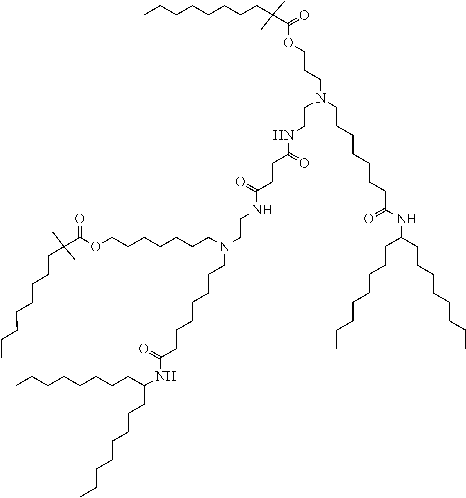 [SMILES: CCCCCCCCC(CCCCCCCC)NC(=O)CCCCCCCN(CCCCCCCOC(=O)C(C)(C)CCCCCCCC)CCNC(=O)CCC(=O)NCCN(CCCCCCCC(=O)NC(CCCCCCCC)CCCCCCCC)CCCOC(=O)C(C)(C)CCCCCCCC] |
| 2571 | 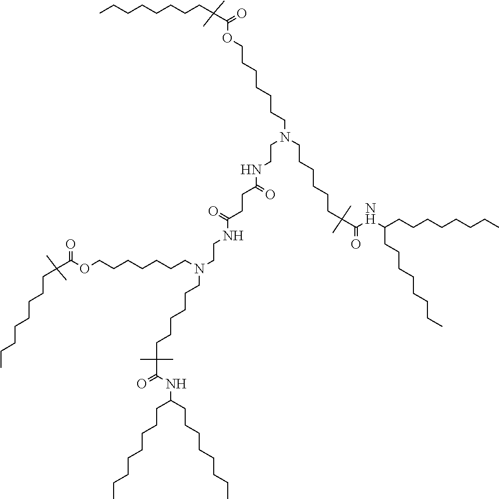 [SMILES: CCCCCCCCC(CCCCCCCC)NC(=O)C(C)(C)CCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)NC(CCCCCCCC)CCCCCCCC] |
| 2573 | 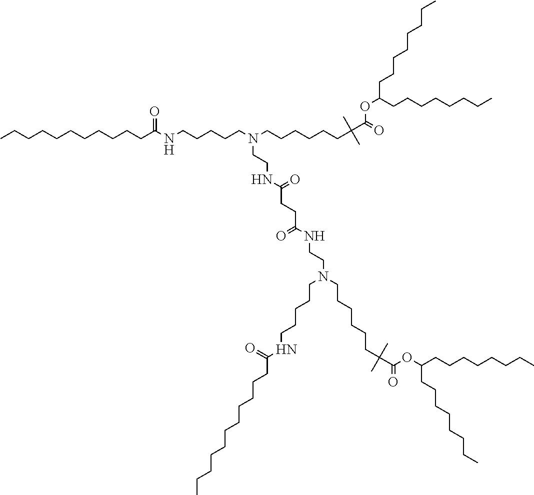 [SMILES: CCCCCCCCCCCC(=O)NCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCNC(=O)CCCCCCCCCCC] |
| 2574 | 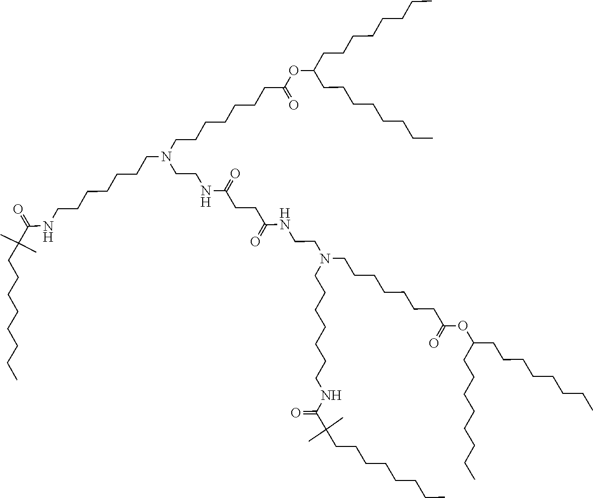 [SMILES: CCCCCCCCC(CCCCCCCC)OC(=O)CCCCCCCN(CCCCCCCNC(=O)C(C)(C)CCCCCCCC)CCNC(=O)CCC(=O)NCCN(CCCCCCCNC(=O)C(C)(C)CCCCCCCC)CCCCCCCC(=O)OC(CCCCCCCC)CCCCCCCC] |
| 2575 | 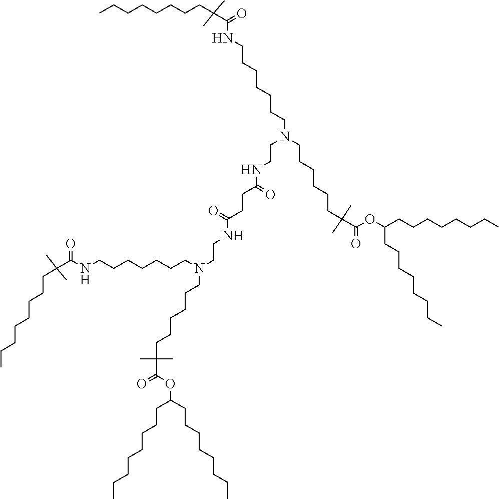 [SMILES: CCCCCCCCC(CCCCCCCC)OC(=O)C(C)(C)CCCCCCN(CCCCCCCNC(=O)C(C)(C)CCCCCCCC)CCNC(=O)CCC(=O)NCCN(CCCCCCCNC(=O)C(C)(C)CCCCCCCC)CCCCCCC(C)(C)C(=O)OC(CCCCCCCC)CCCCCCCC] |
| 2577 | 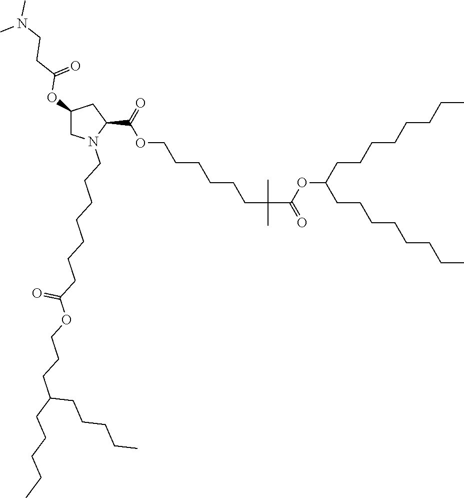 [SMILES: CCCCCCCCC(CCCCCCCC)OC(=O)C(C)(C)CCCCCCOC(=O)[C@@H]1C[C@H](OC(=O)CCN(C)C)CN1CCCCCCCC(=O)OCCCC(CCCCC)CCCCC] |
| 2578 | 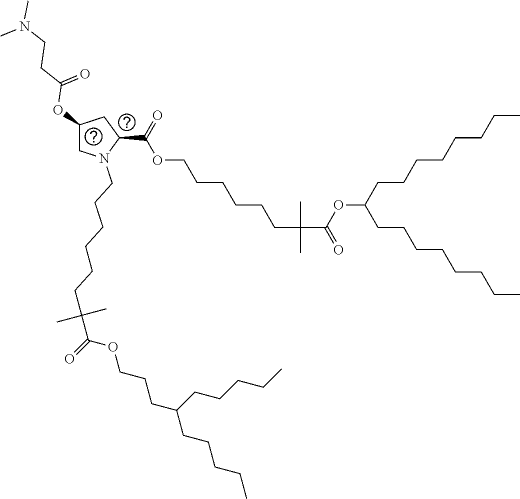 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)OCCCC(CCCCC)CCCCC] |
| 2580 | 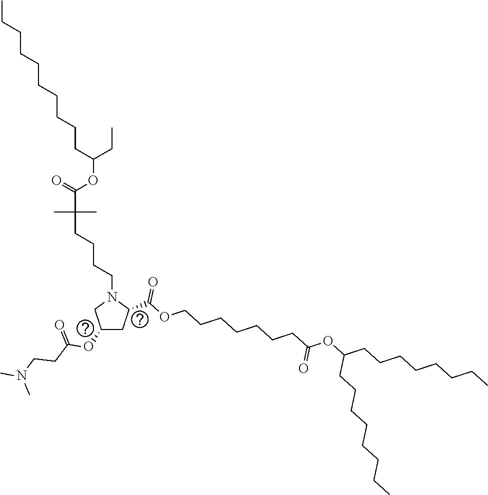 [SMILES: CCCCCCCCCCC(CC)OC(=O)C(C)(C)CCCCN1C[C@@H](OC(=O)CCN(C)C)C[C@H]1C(=O)OCCCCCCCC(=O)OC(CCCCCCCC)CCCCCCCC] |
| 2581 | 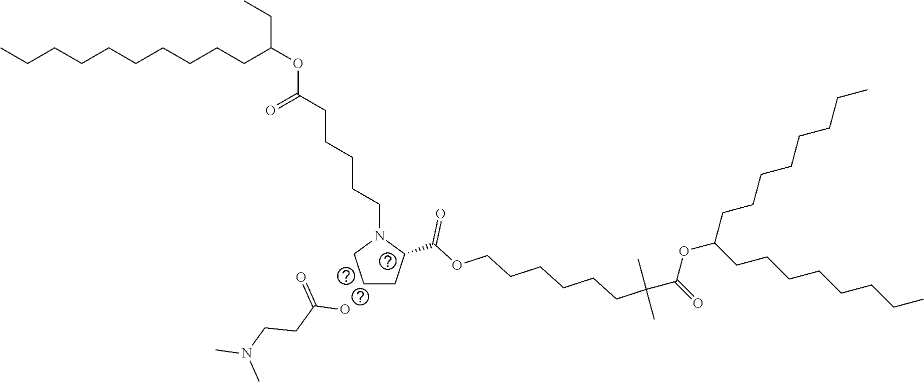 |
| 2582 | 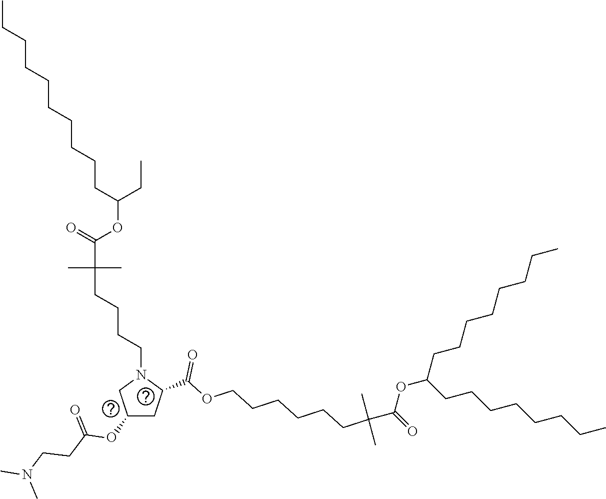 [SMILES: CCCCCCCCCCC(CC)OC(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] |
| 2583 | 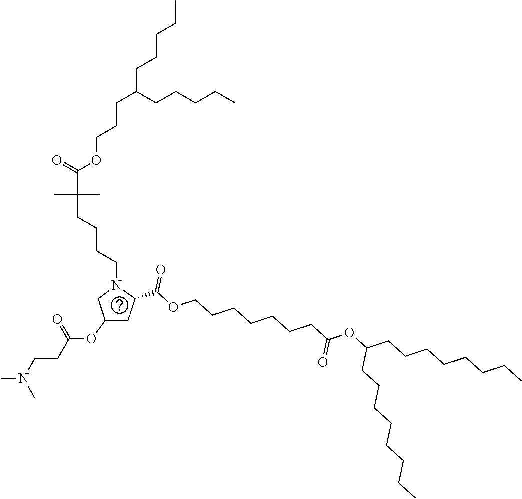 [SMILES: CCCCCCCCC(CCCCCCCC)OC(=O)CCCCCCCOC(=O)[C@@H]1CC(OC(=O)CCN(C)C)CN1CCCCC(C)(C)C(=O)OCCCC(CCCCC)CCCCC] |
| 2584 | 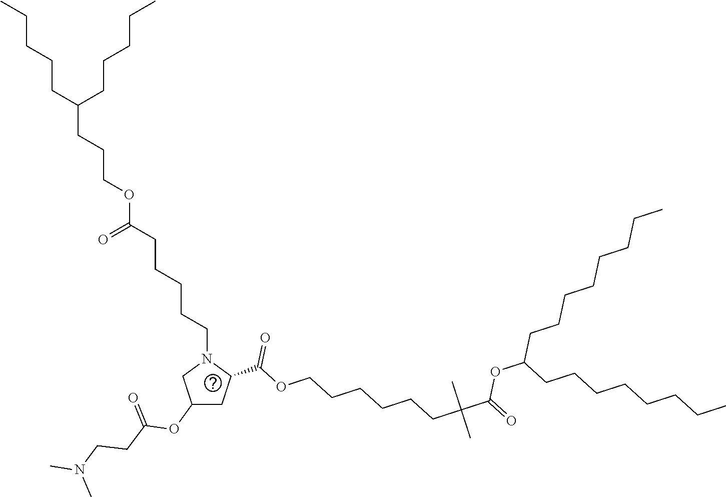 [SMILES: CCCCCCCCC(CCCCCCCC)OC(=O)C(C)(C)CCCCCCOC(=O)[C@@H]1CC(OC(=O)CCN(C)C)CN1CCCCCC(=O)OCCCC(CCCCC)CCCCC] |
| 2585 | 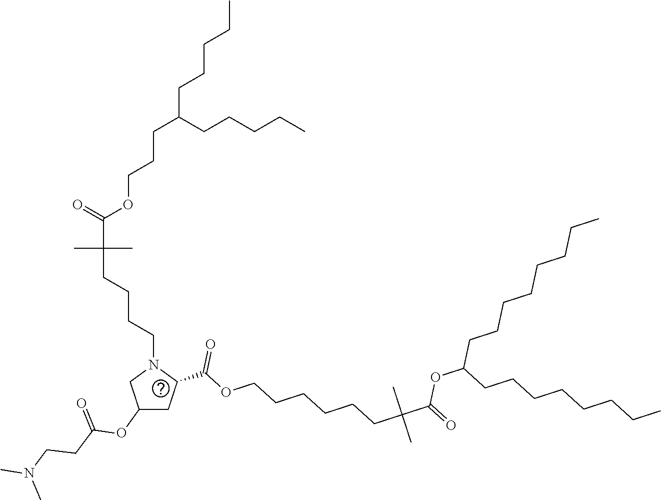 [SMILES: CCCCCCCCC(CCCCCCCC)OC(=O)C(C)(C)CCCCCCOC(=O)[C@@H]1CC(OC(=O)CCN(C)C)CN1CCCCC(C)(C)C(=O)OCCCC(CCCCC)CCCCC] |
| 2586 | 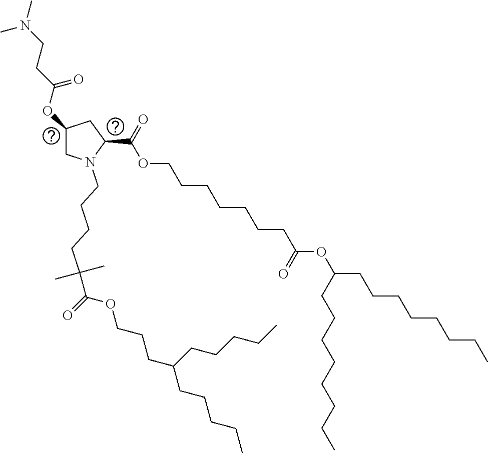 [SMILES: CCCCCCCCC(CCCCCCCC)OC(=O)CCCCCCCOC(=O)[C@@H]1C[C@H](OC(=O)CCN(C)C)CN1CCCCC(C)(C)C(=O)OCCCC(CCCCC)CCCCC] |
| 2587 | 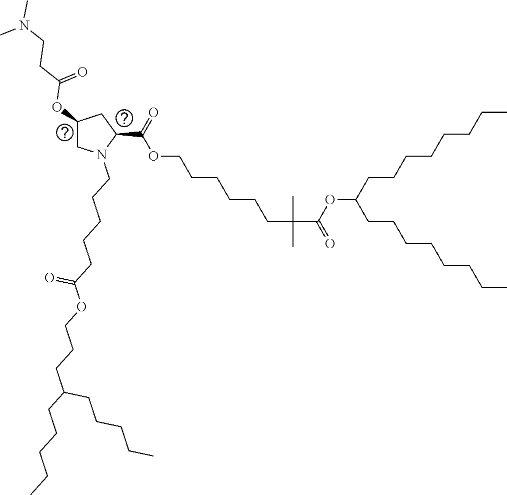 [SMILES: CCCCCCCCC(CCCCCCCC)OC(=O)C(C)(C)CCCCCCOC(=O)[C@@H]1C[C@H](OC(=O)CCN(C)C)CN1CCCCCC(=O)OCCCC(CCCCC)CCCCC] |
| 2588 | 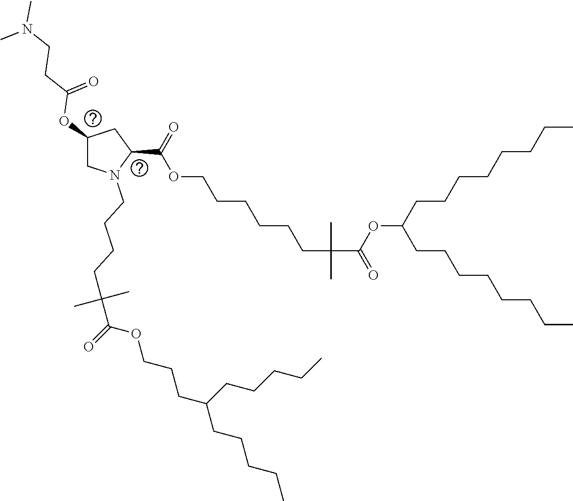 [SMILES: CCCCCCCCC(CCCCCCCC)OC(=O)C(C)(C)CCCCCCOC(=O)[C@@H]1C[C@H](OC(=O)CCN(C)C)CN1CCCCC(C)(C)C(=O)OCCCC(CCCCC)CCCCC] |
| 2589 | 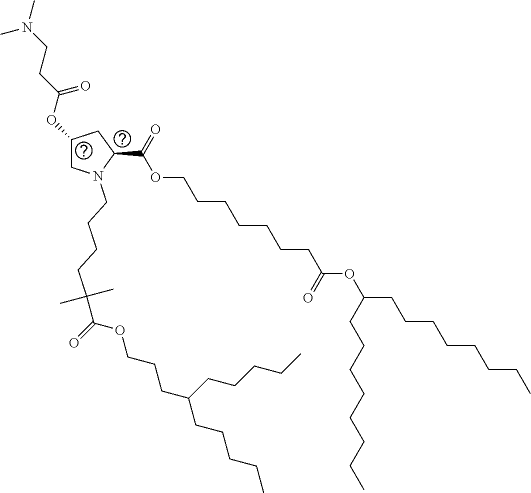 [SMILES: CCCCCCCCC(CCCCCCCC)OC(=O)CCCCCCCOC(=O)[C@@H]1C[C@@H](OC(=O)CCN(C)C)CN1CCCCC(C)(C)C(=O)OCCCC(CCCCC)CCCCC] |
| 2590 | 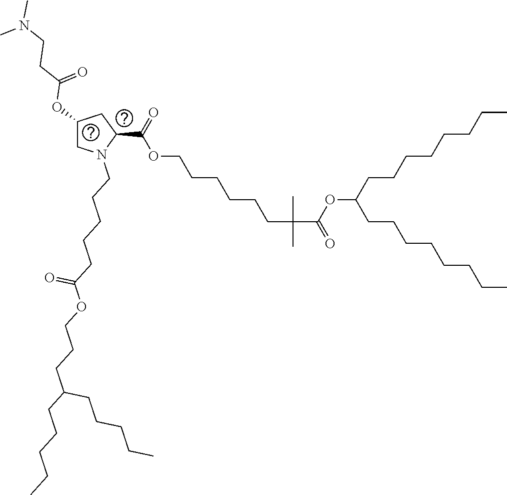 [SMILES: CCCCCCCCC(CCCCCCCC)OC(=O)C(C)(C)CCCCCCOC(=O)[C@@H]1C[C@@H](OC(=O)CCN(C)C)CN1CCCCCC(=O)OCCCC(CCCCC)CCCCC] |
| 2591 | 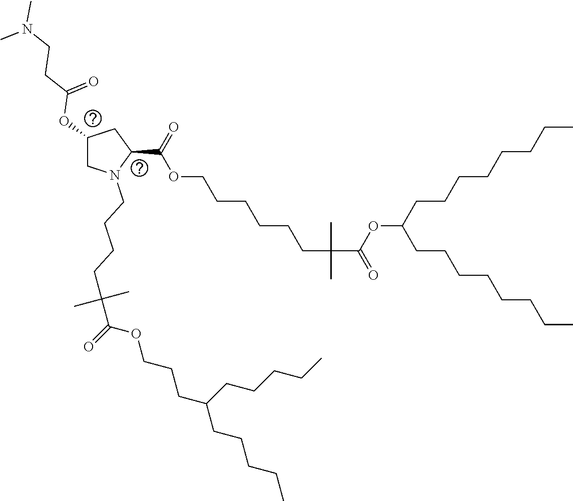 [SMILES: CCCCCCCCC(CCCCCCCC)OC(=O)C(C)(C)CCCCCCOC(=O)[C@@H]1C[C@@H](OC(=O)CCN(C)C)CN1CCCCC(C)(C)C(=O)OCCCC(CCCCC)CCCCC] |
| 2592 | 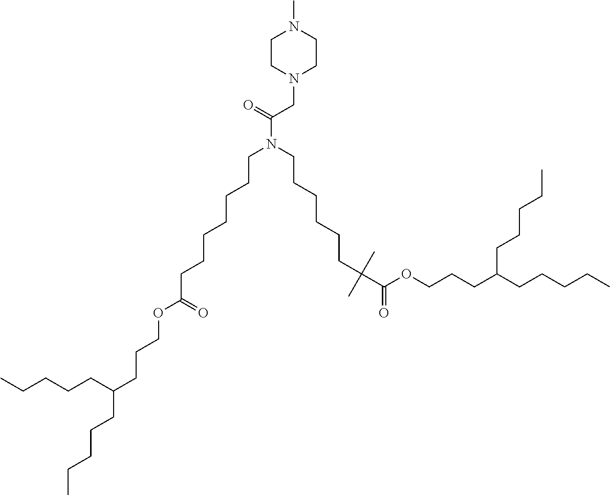 [SMILES: CCCCCC(CCCCC)CCCOC(=O)CCCCCCCN(CCCCCCC(C)(C)C(=O)OCCCC(CCCCC)CCCCC)C(=O)CN1CCN(C)CC1] |
| 2599 | 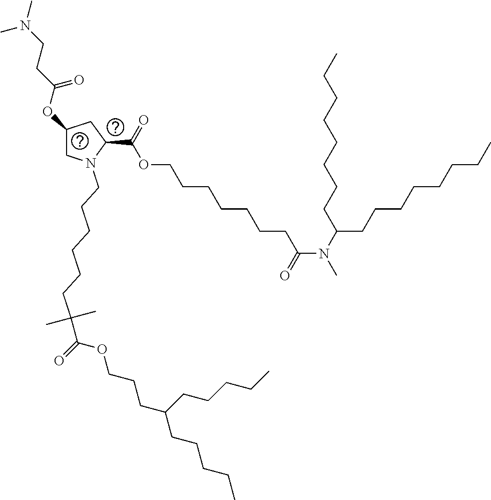 [SMILES: CCCCCCCCC(CCCCCCCC)N(C)C(=O)CCCCCCCOC(=O)[C@@H]1C[C@H](OC(=O)CCN(C)C)CN1CCCCCCC(C)(C)C(=O)OCCCC(CCCCC)CCCCC] |
| 2600 | 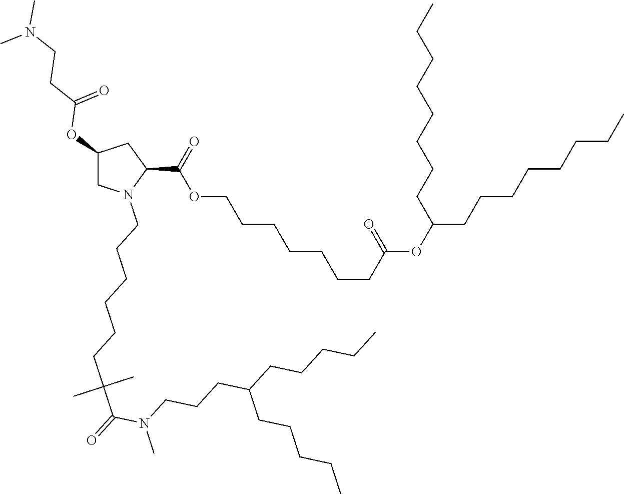 [SMILES: CCCCCCCCC(CCCCCCCC)OC(=O)CCCCCCCOC(=O)[C@@H]1C[C@H](OC(=O)CCN(C)C)CN1CCCCCCC(C)(C)C(=O)N(C)CCCC(CCCCC)CCCCC] |
| 2601 | 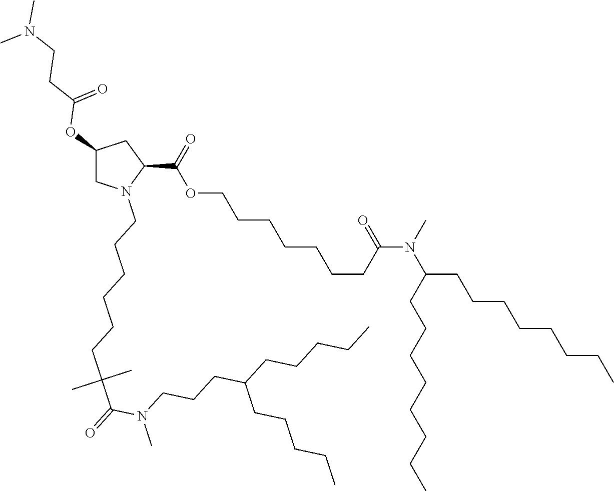 [SMILES: CCCCCCCCC(CCCCCCCC)N(C)C(=O)CCCCCCCOC(=O)[C@@H]1C[C@H](OC(=O)CCN(C)C)CN1CCCCCCC(C)(C)C(=O)N(C)CCCC(CCCCC)CCCCC] |
| 2608 | 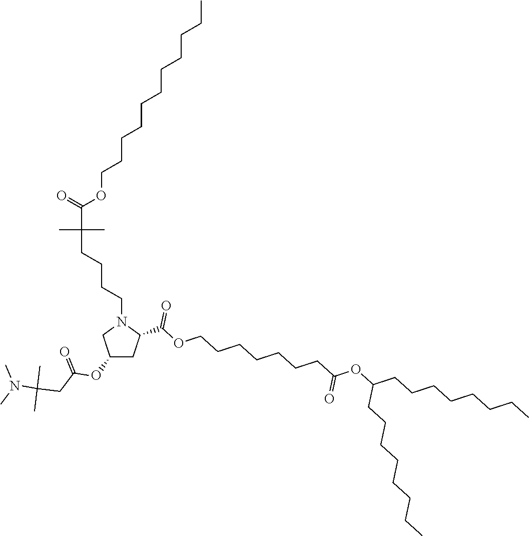 [SMILES: CCCCCCCCCCCOC(=O)C(C)(C)CCCCN1C[C@@H](OC(=O)CC(C)(C)N(C)C)C[C@H]1C(=O)OCCCCCCCC(=O)OC(CCCCCCCC)CCCCCCCC] |
| 2609 | 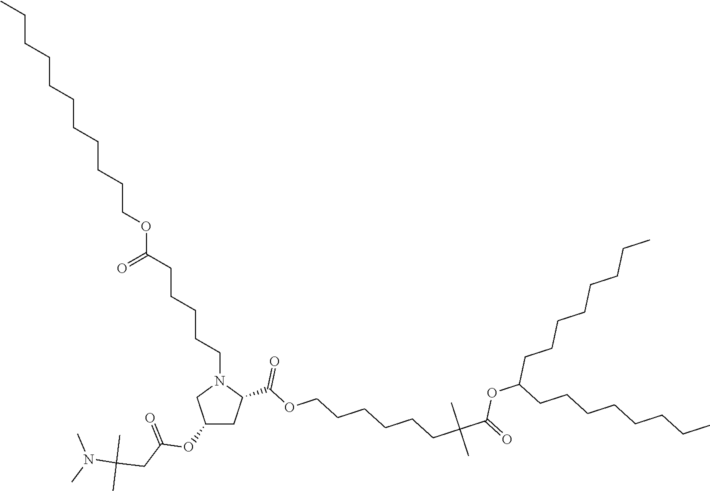 [SMILES: CCCCCCCCCCCOC(=O)CCCCCN1C[C@@H](OC(=O)CC(C)(C)N(C)C)C[C@H]1C(=O)OCCCCCCC(C)(C)C(=O)OC(CCCCCCCC)CCCCCCCC] |
| 2610 | 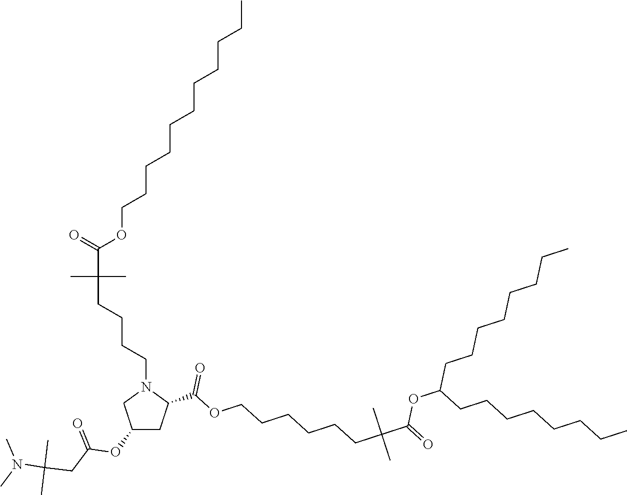 [SMILES: CCCCCCCCCCCOC(=O)C(C)(C)CCCCN1C[C@@H](OC(=O)CC(C)(C)N(C)C)C[C@H]1C(=O)OCCCCCCC(C)(C)C(=O)OC(CCCCCCCC)CCCCCCCC] |
| 2611 | 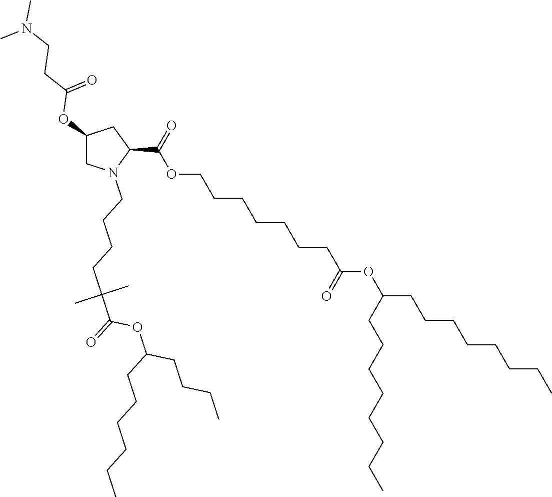 [SMILES: CCCCCCCCC(CCCCCCCC)OC(=O)CCCCCCCOC(=O)[C@@H]1C[C@H](OC(=O)CCN(C)C)CN1CCCCC(C)(C)C(=O)OC(CCCC)CCCCCC] |
| 2612 | 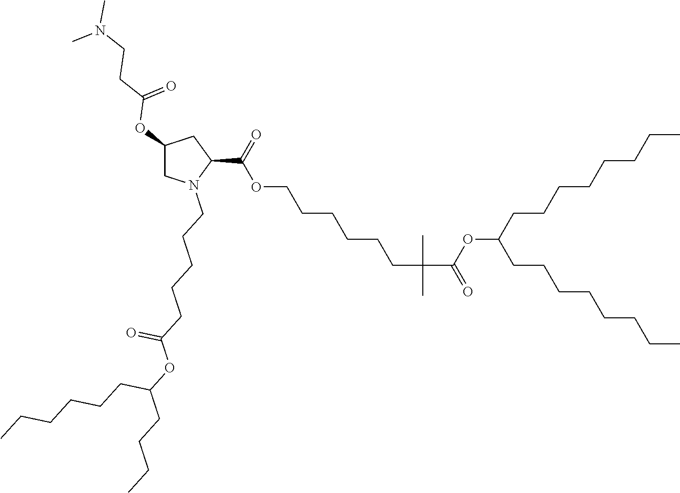 [SMILES: CCCCCCCCC(CCCCCCCC)OC(=O)C(C)(C)CCCCCCOC(=O)[C@@H]1C[C@H](OC(=O)CCN(C)C)CN1CCCCCC(=O)OC(CCCC)CCCCCC] |
| 2613 | 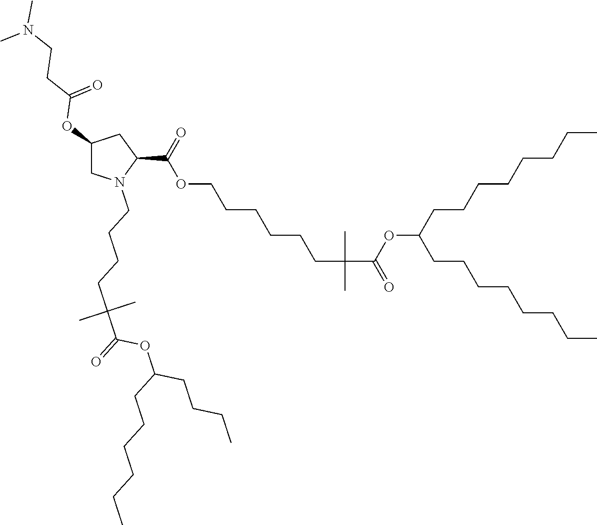 [SMILES: CCCCCCCCC(CCCCCCCC)OC(=O)C(C)(C)CCCCCCOC(=O)[C@@H]1C[C@H](OC(=O)CCN(C)C)CN1CCCCC(C)(C)C(=O)OC(CCCC)CCCCCC] |
| A1 | 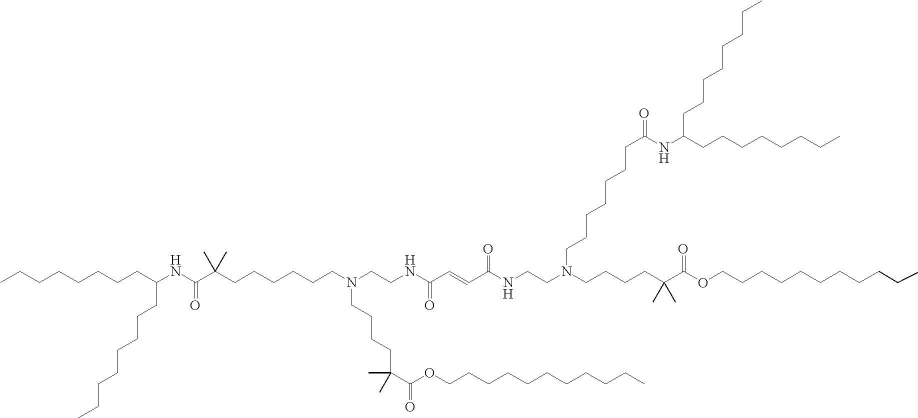 [SMILES: CCCCCCCCCCCOC(=O)C(C)(C)CCCCN(CCCCCCCC(=O)NC(CCCCCCCC)CCCCCCCC)CCNC(=O)/C=C/C(=O)NCCN(CCCCCCC(C)(C)C(=O)NC(CCCCCCCC)CCCCCCCC)CCCCC(C)(C)C(=O)OCCCCCCCCCCC] |
| A2 | 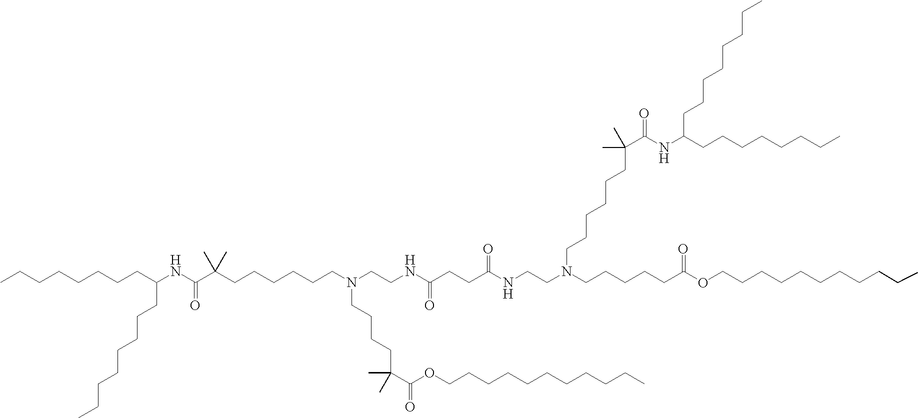 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] |
| A3 | 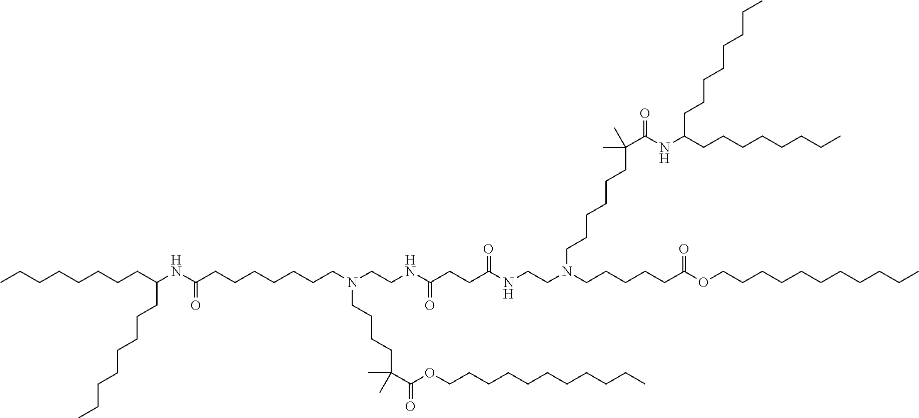 [SMILES: CCCCCCCCCCCOC(=O)CCCCCN(CCCCCCC(C)(C)C(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] |
| A4 | 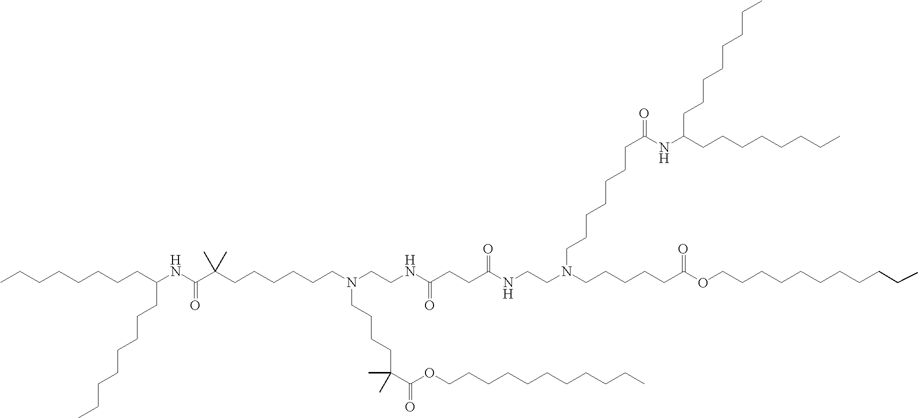 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)OCCCCCCCCCCC] |
| A5 | 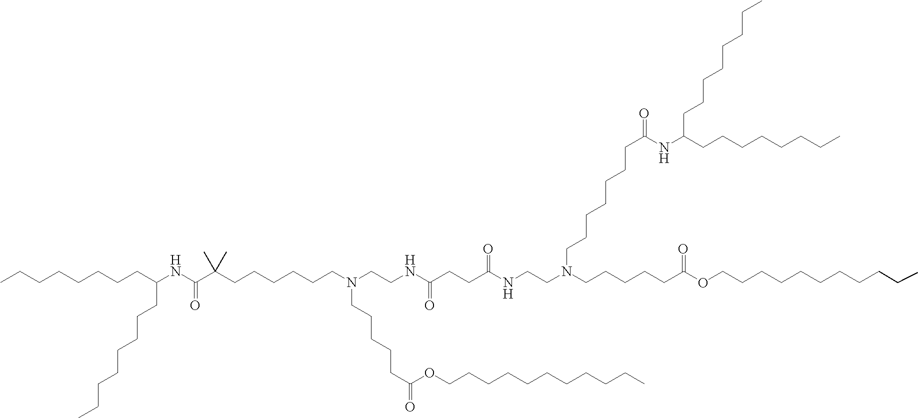 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCCC(=O)OCCCCCCCCCCC] |
| A6 | 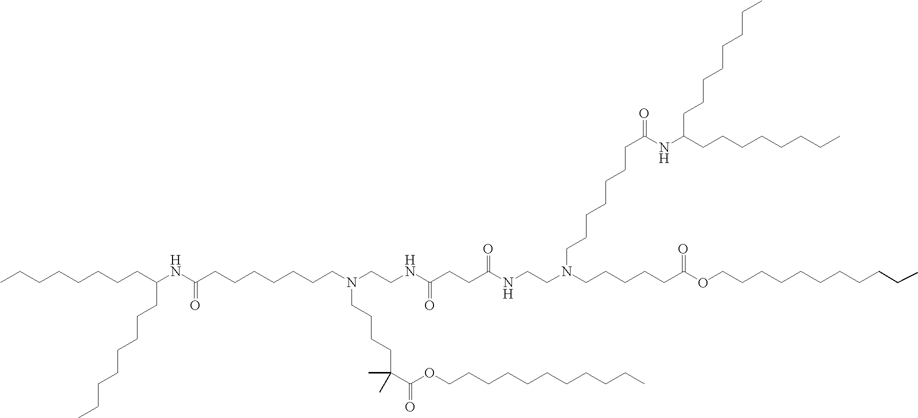 [SMILES: CCCCCCCCCCCOC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)OCCCCCCCCCCC] |
| A7 | 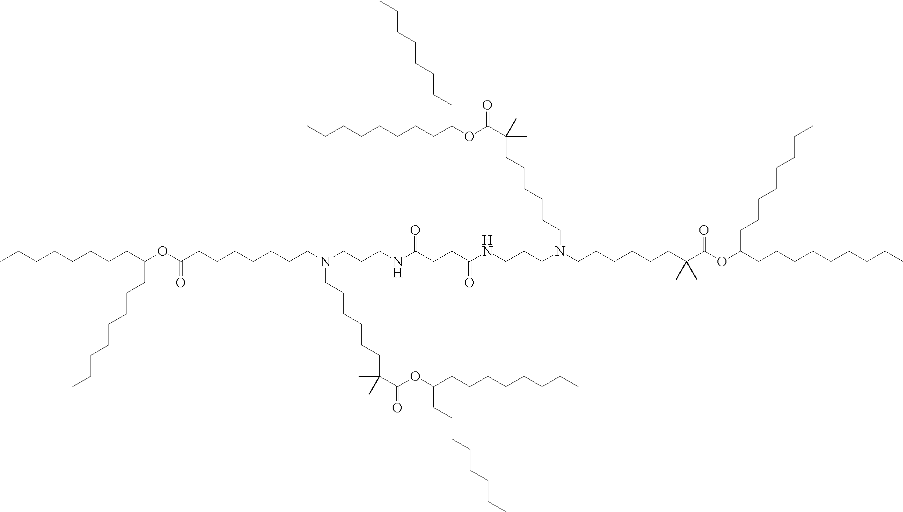 [SMILES: CCCCCCCCCC(CCCCCCCC)OC(=O)C(C)(C)CCCCCCN(CCCCCCC(C)(C)C(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] |
| A8 | 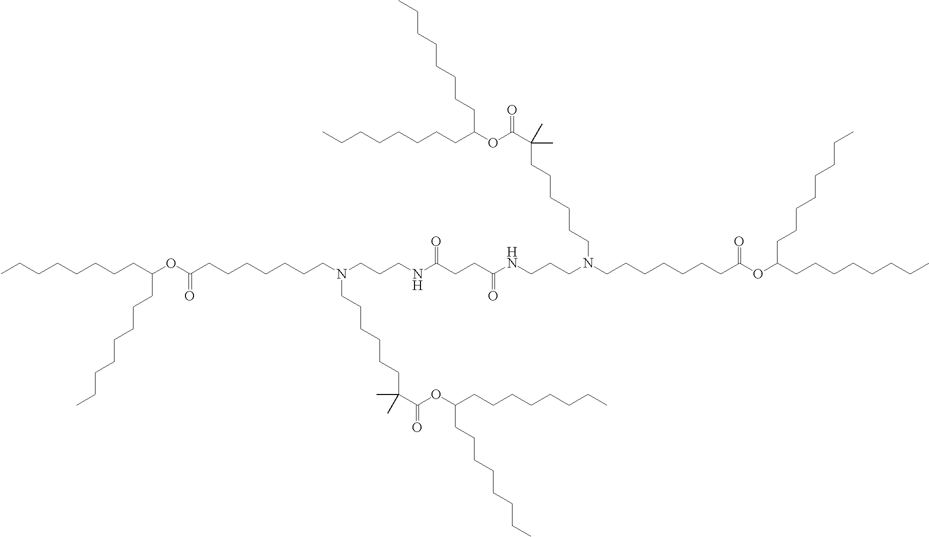 [SMILES: CCCCCCCCC(CCCCCCCC)OC(=O)CCCCCCCN(CCCCCCC(C)(C)C(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] |
| A9 | 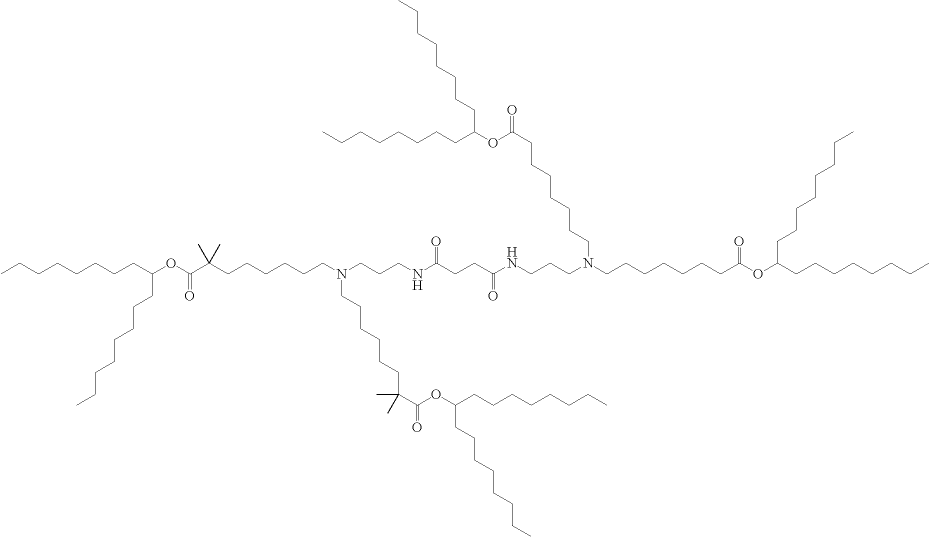 [SMILES: CCCCCCCCC(CCCCCCCC)OC(=O)CCCCCCCN(CCCCCCCC(=O)OC(CCCCCCCC)CCCCCCCC)CCCNC(=O)CCC(=O)NCCCN(CCCCCCC(C)(C)C(=O)OC(CCCCCCCC)CCCCCCCC)CCCCCCC(C)(C)C(=O)OC(CCCCCCCC)CCCCCCCC] |
| A10 | 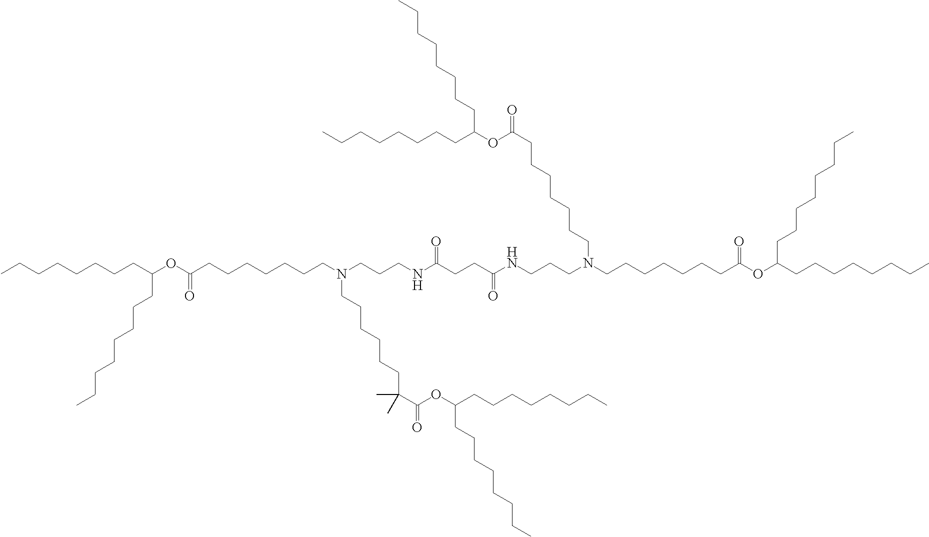 [SMILES: CCCCCCCCC(CCCCCCCC)OC(=O)CCCCCCCN(CCCCCCCC(=O)OC(CCCCCCCC)CCCCCCCC)CCCNC(=O)CCC(=O)NCCCN(CCCCCCCC(=O)OC(CCCCCCCC)CCCCCCCC)CCCCCCC(C)(C)C(=O)OC(CCCCCCCC)CCCCCCCC] |
| A11 | 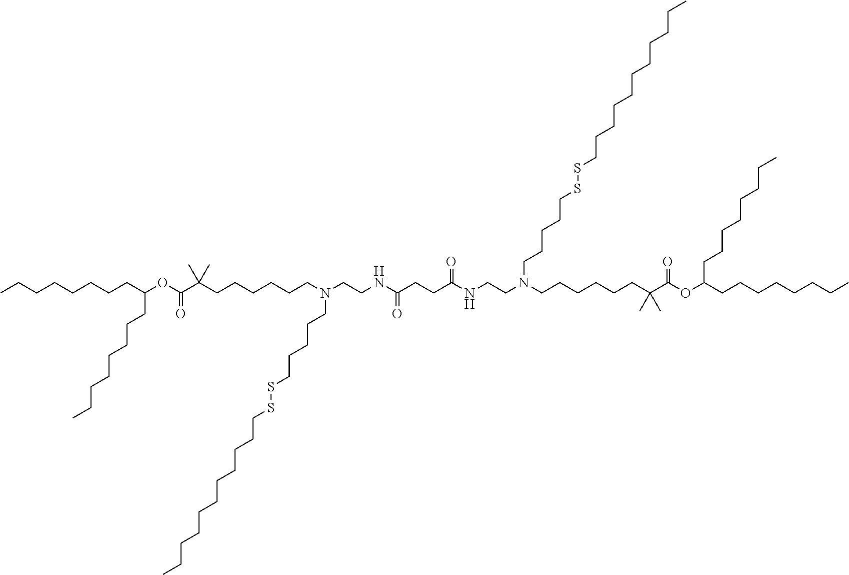 [SMILES: CCCCCCCCCCCSSCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCSSCCCCCCCCCCC] |
| A12 | 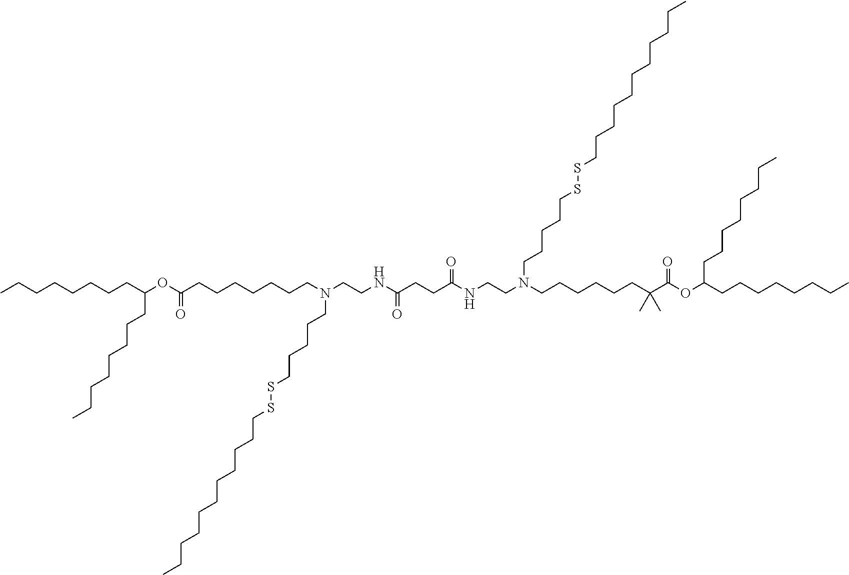 [SMILES: CCCCCCCCCCCSSCCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCSSCCCCCCCCCCC] |
| A13 | 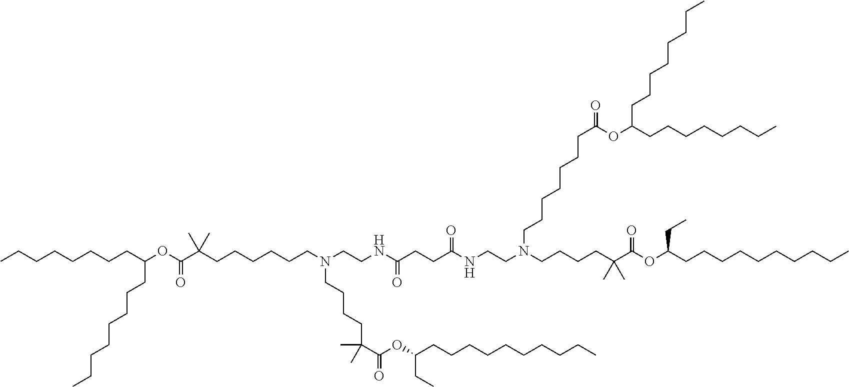 [SMILES: CCCCCCCCCC[C@H](CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] |
| A14 | 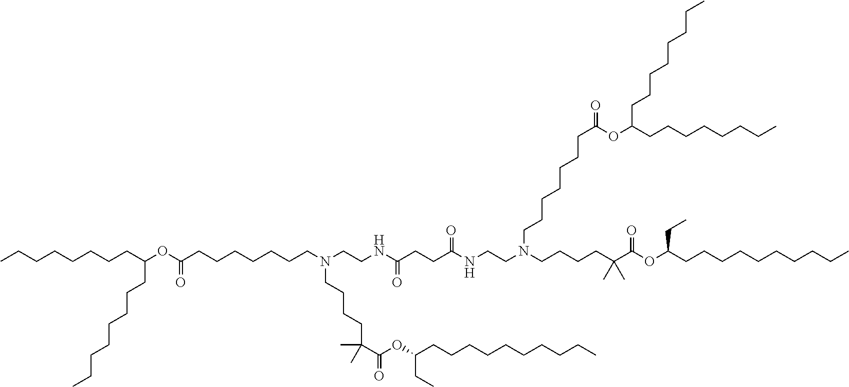 [SMILES: CCCCCCCCCC[C@H](CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] |
| A15 | 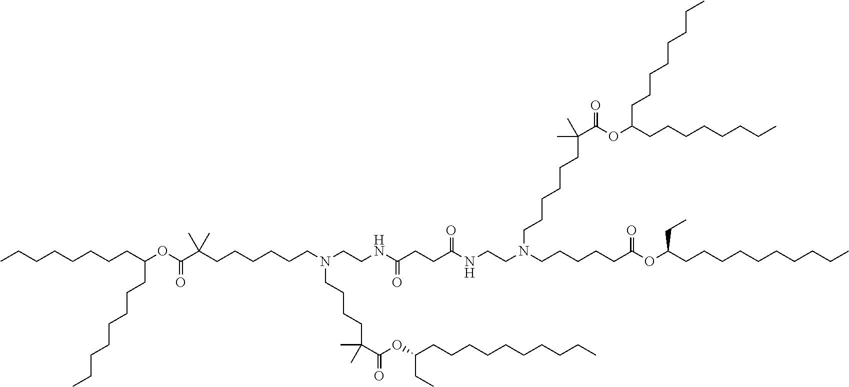 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] |
| A16 | 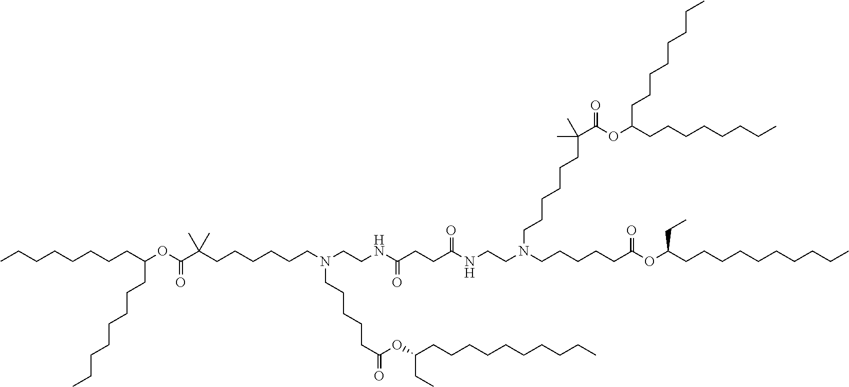 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@@H](CC)CCCCCCCCCC] |
| A17 | 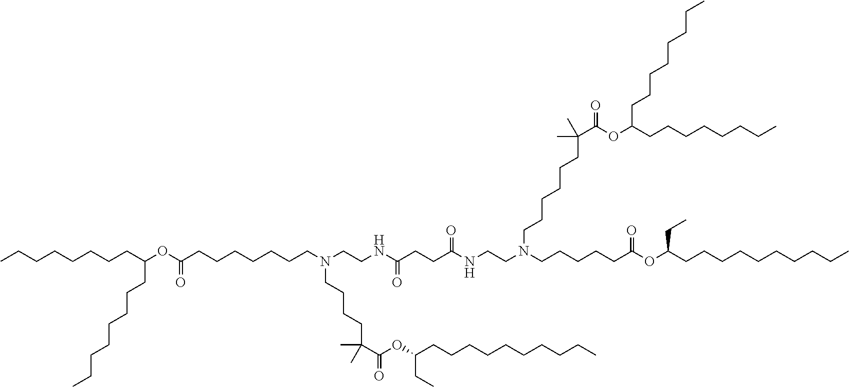 [SMILES: CCCCCCCCCC[C@H](CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] |
| A18 | 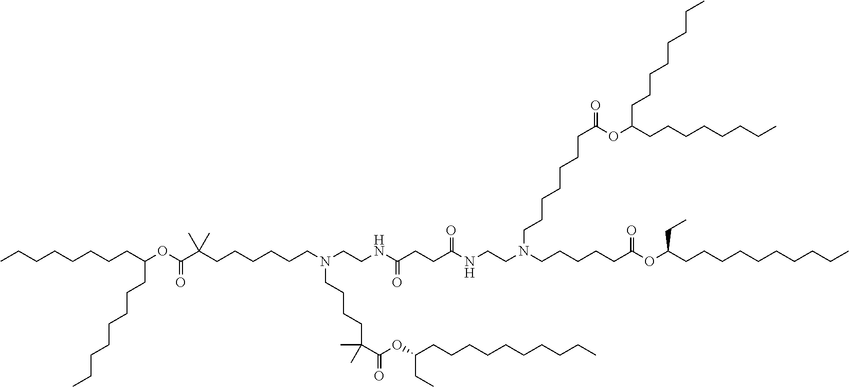 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@@H](CC)CCCCCCCCCC] |
| A19 | 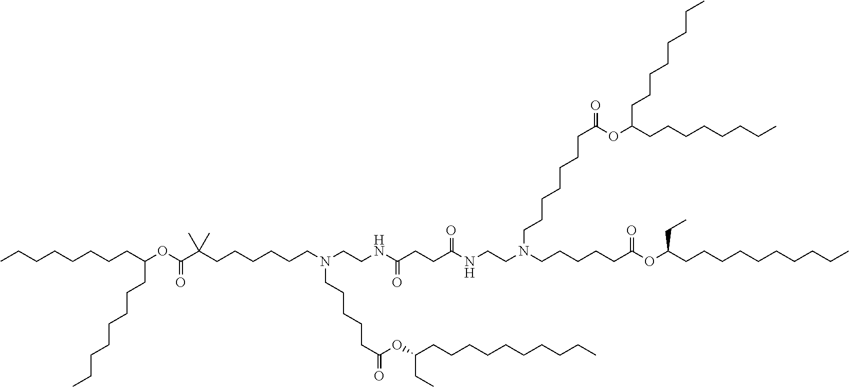 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@@H](CC)CCCCCCCCCC] |
| A20 | 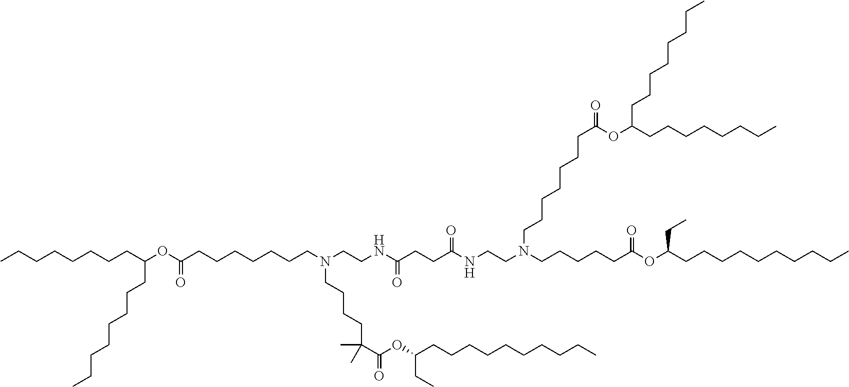 [SMILES: CCCCCCCCCC[C@H](CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@@H](CC)CCCCCCCCCC] |
| A21 | 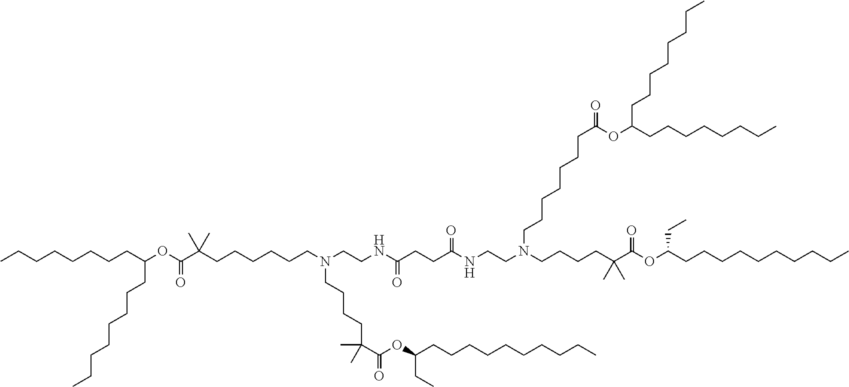 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] |
| A22 | 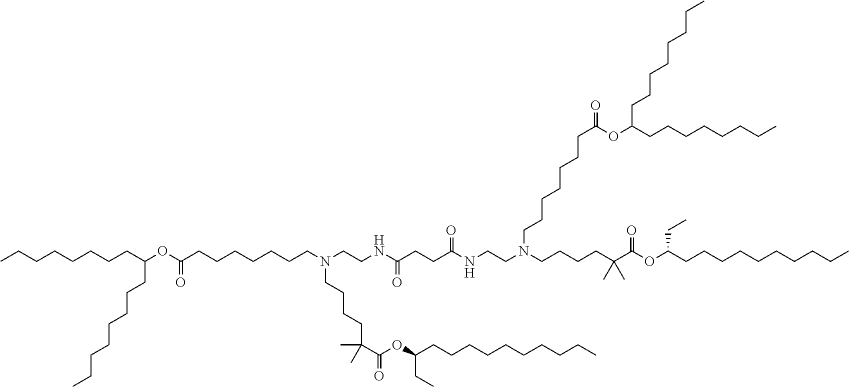 [SMILES: CCCCCCCCCC[C@@H](CC)OC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] |
| A23 | 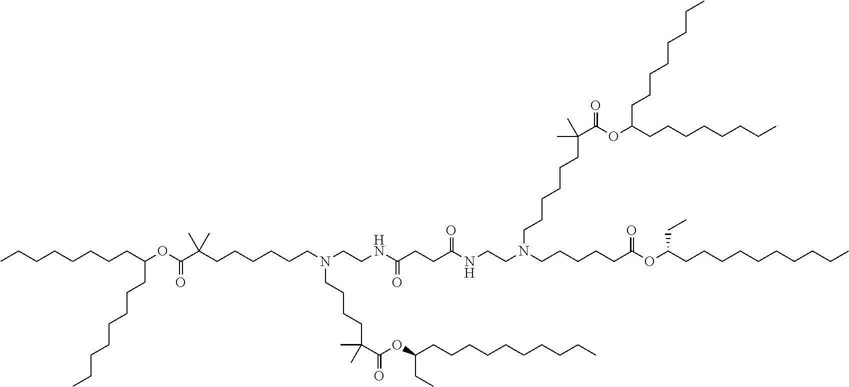 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] |
| A24 | 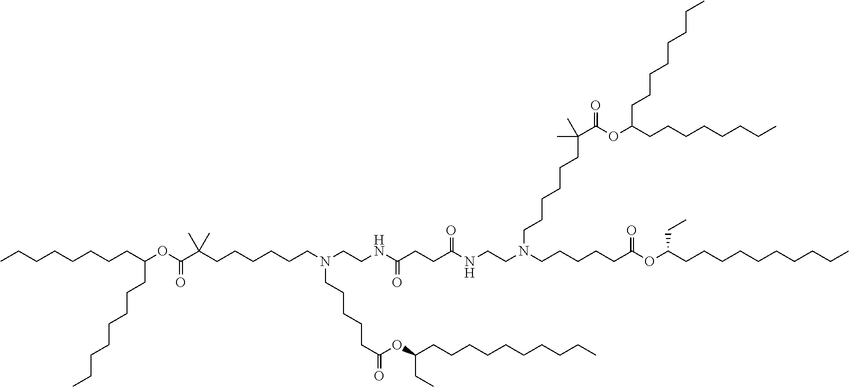 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@H](CC)CCCCCCCCCC] |
| A25 | 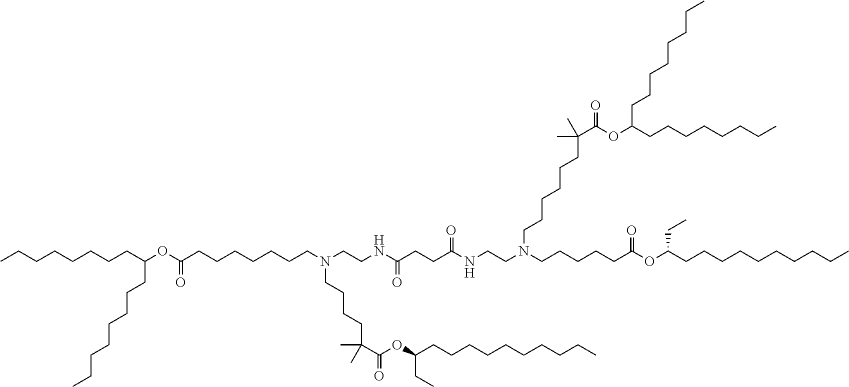 [SMILES: CCCCCCCCCC[C@@H](CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)O[C@H](CC)CCCCCCCCCC] |
| A26 | 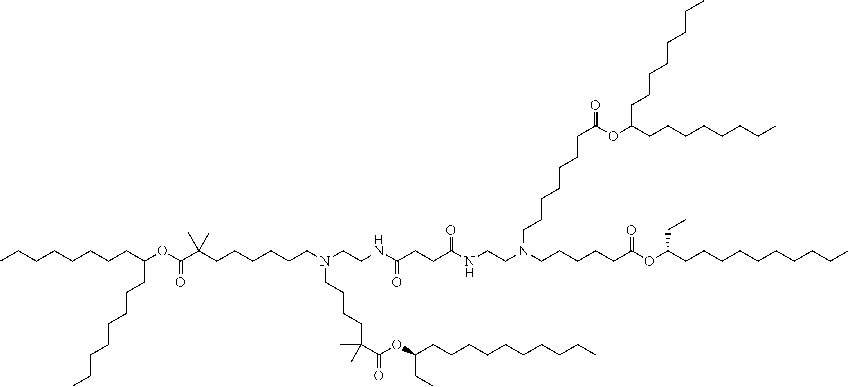 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)O[C@H](CC)CCCCCCCCCC] |
| A27 | 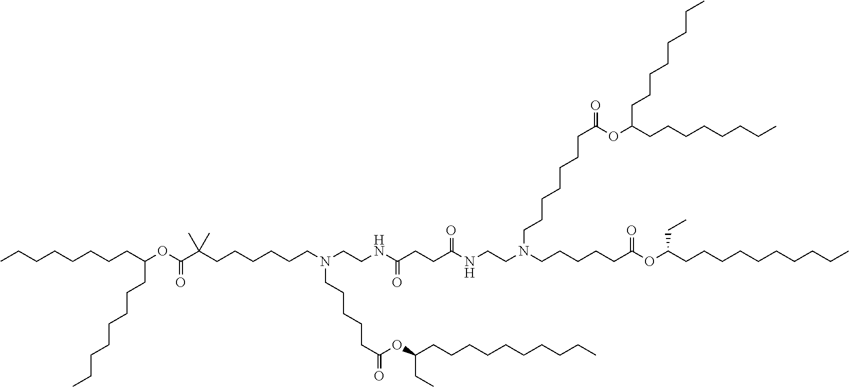 [SMILES: CCCCCCCCCC[C@@H](CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@H](CC)CCCCCCCCCC] |
| A28 | 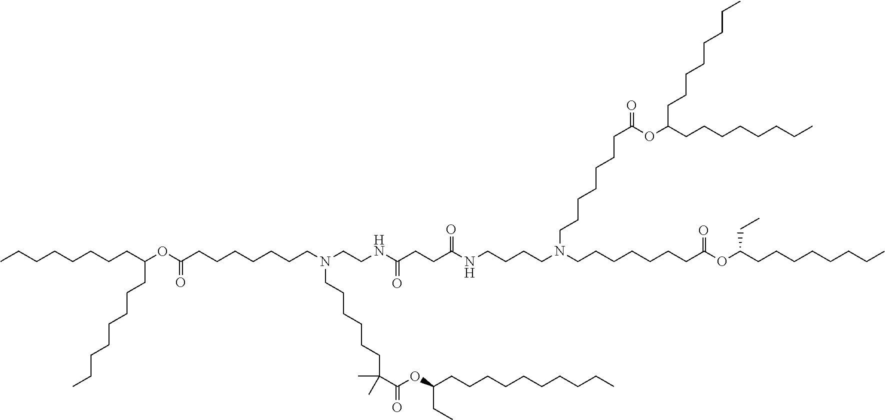 [SMILES: CCCCCCCCCC[C@@H](CC)OC(=O)C(C)(C)CCCCCCN(CCCCCCCC(=O)OC(CCCCCCCC)CCCCCCCC)CCNC(=O)CCC(=O)NCCCCN(CCCCCCCC(=O)OC(CCCCCCCC)CCCCCCCC)CCCCCCCC(=O)O[C@H](CC)CCCCCCCC] |
| A29 | 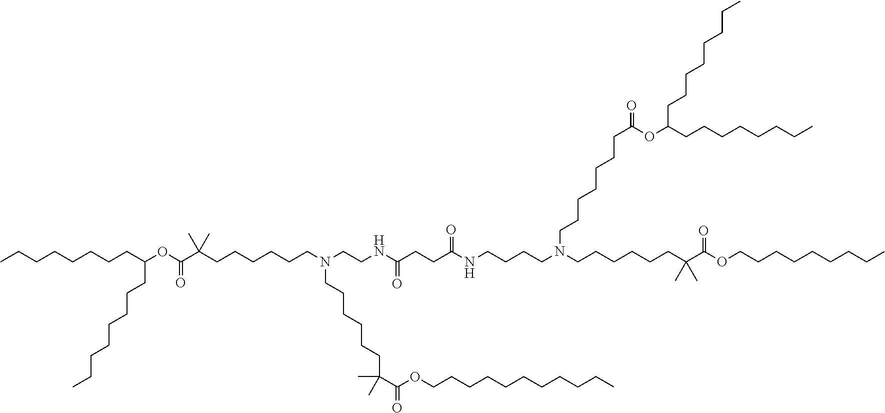 [SMILES: CCCCCCCCCCCOC(=O)C(C)(C)CCCCCCN(CCCCCCC(C)(C)C(=O)OC(CCCCCCCC)CCCCCCCC)CCNC(=O)CCC(=O)NCCCCN(CCCCCCCC(=O)OC(CCCCCCCC)CCCCCCCC)CCCCCCC(C)(C)C(=O)OCCCCCCCCC] |
| A30 | 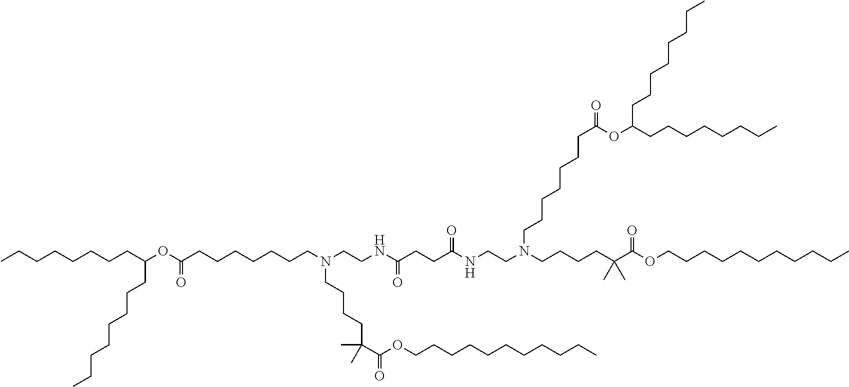 [SMILES: CCCCCCCCCCCOC(=O)C(C)(C)CCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] |
| A31 | 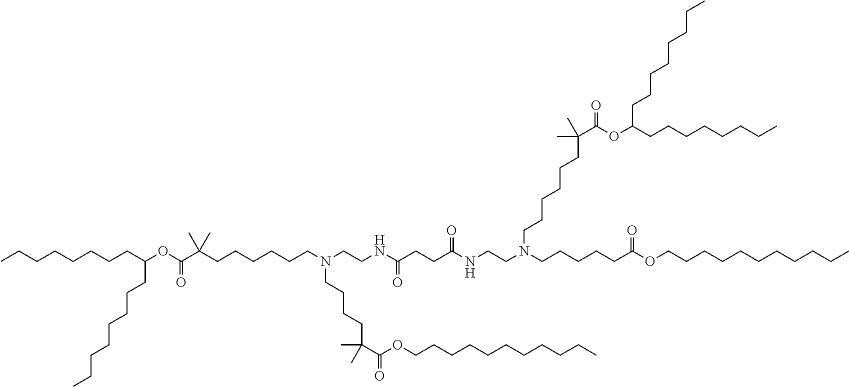 [SMILES: CCCCCCCCCCCOC(=O)CCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] |
| A32 | 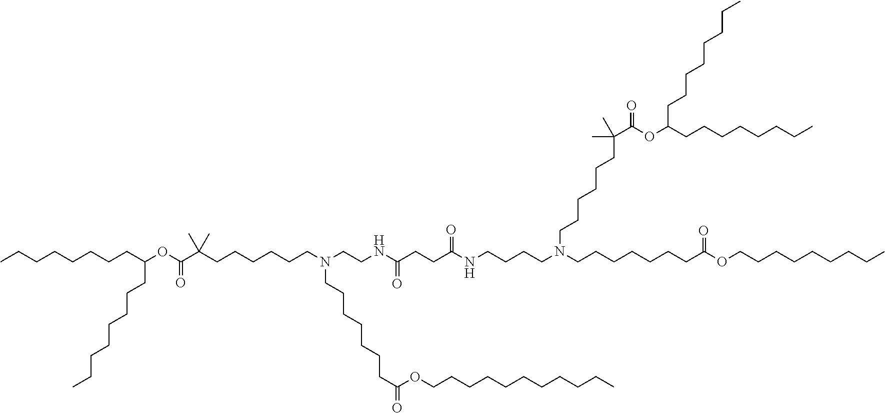 [SMILES: CCCCCCCCCCCOC(=O)CCCCCCCN(CCCCCCC(C)(C)C(=O)OC(CCCCCCCC)CCCCCCCC)CCNC(=O)CCC(=O)NCCCCN(CCCCCCCC(=O)OCCCCCCCCC)CCCCCCC(C)(C)C(=O)OC(CCCCCCCC)CCCCCCCC] |
| A33 | 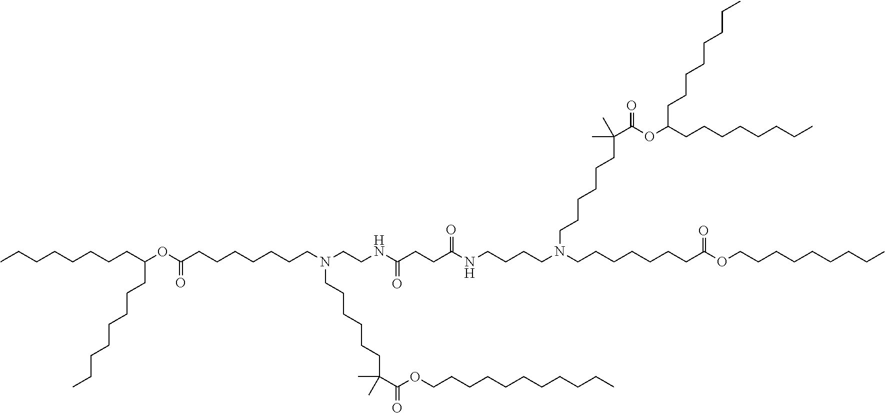 [SMILES: CCCCCCCCCCCOC(=O)C(C)(C)CCCCCCN(CCCCCCCC(=O)OC(CCCCCCCC)CCCCCCCC)CCNC(=O)CCC(=O)NCCCCN(CCCCCCCC(=O)OCCCCCCCCC)CCCCCCC(C)(C)C(=O)OC(CCCCCCCC)CCCCCCCC] |
| A34 | 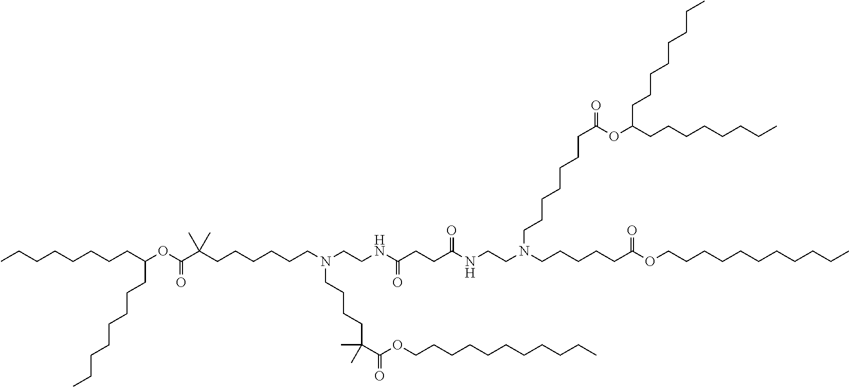 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OCCCCCCCCCCC] |
| A35 | 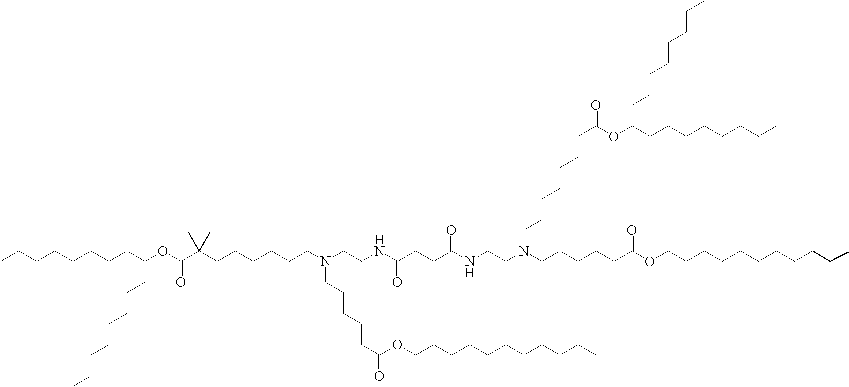 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OCCCCCCCCCCC] |
| A36 | 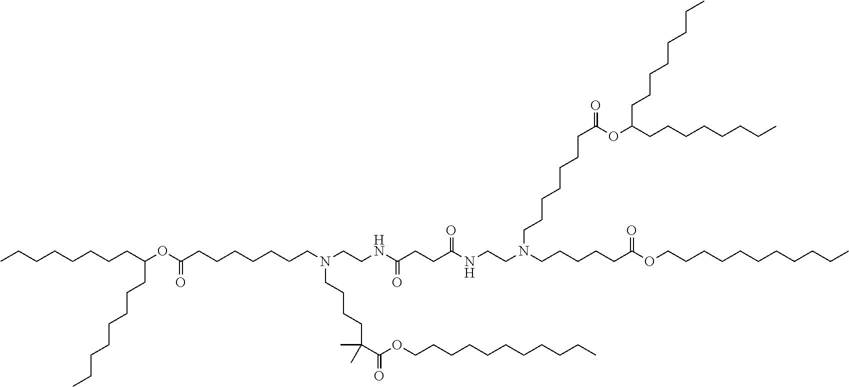 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] |
| A37 | 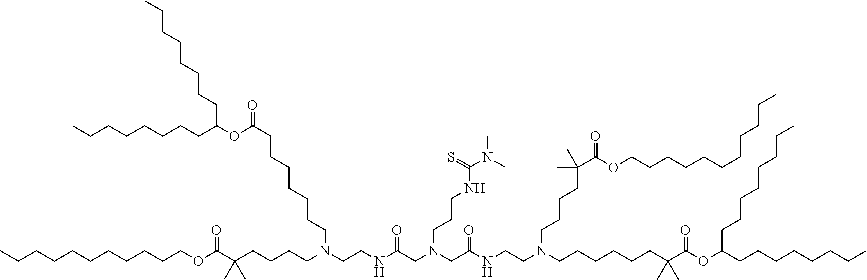 [SMILES: CCCCCCCCCCCOC(=O)C(C)(C)CCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] |
| A38 | 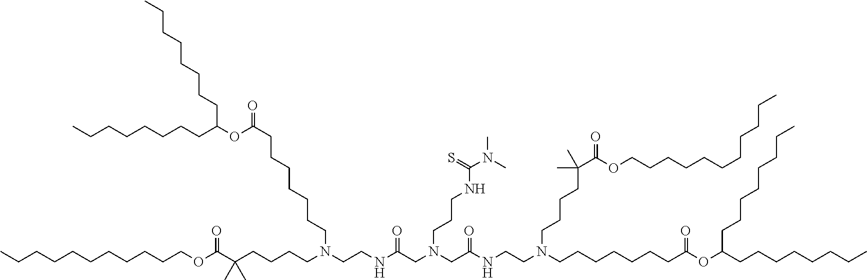 [SMILES: CCCCCCCCCCCOC(=O)C(C)(C)CCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] |
| A39 | 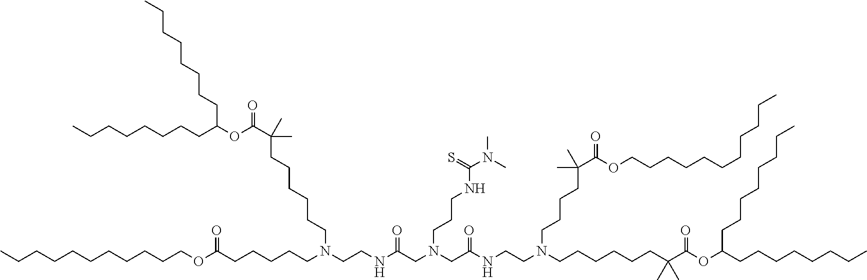 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] |
| A40 | 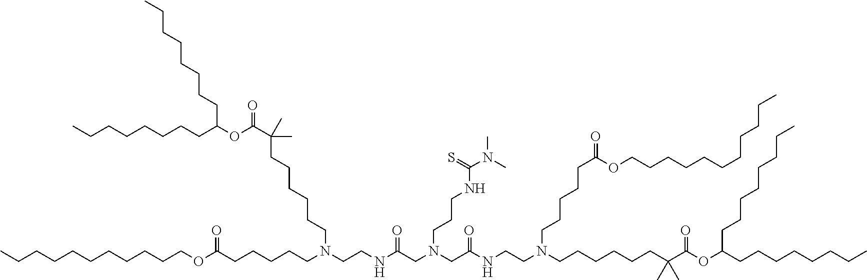 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCCC(=O)OCCCCCCCCCCC] |
| A41 | 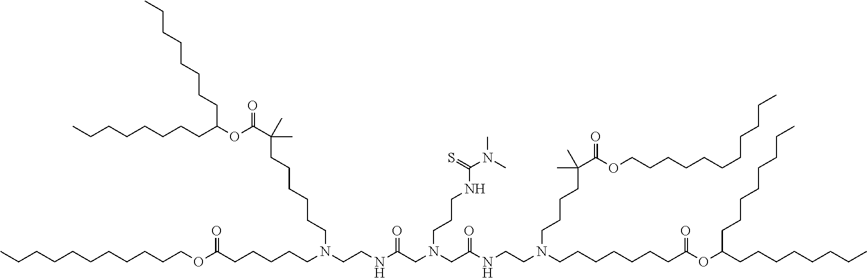 [SMILES: CCCCCCCCCCCOC(=O)CCCCCN(CCCCCCC(C)(C)C(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] |
| A42 | 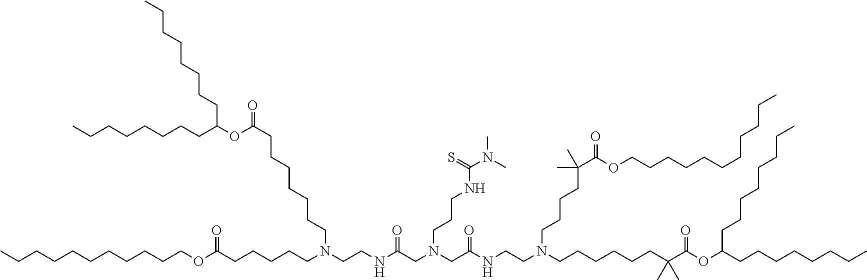 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCC(C)(C)C(=O)OCCCCCCCCCCC] |
| A43 | 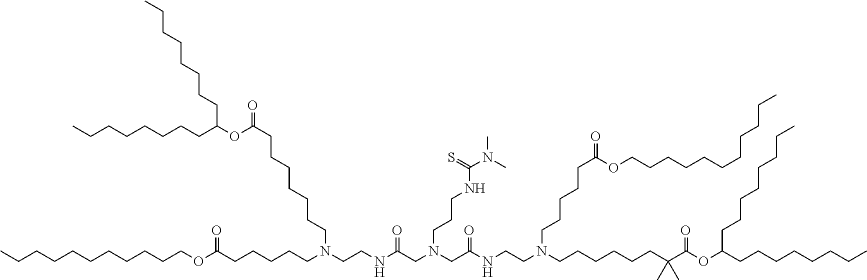 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCC(C)(C)C(=O)OC(CCCCCCCC)CCCCCCCC)CCCCCC(=O)OCCCCCCCCCCC] |
| A44 | 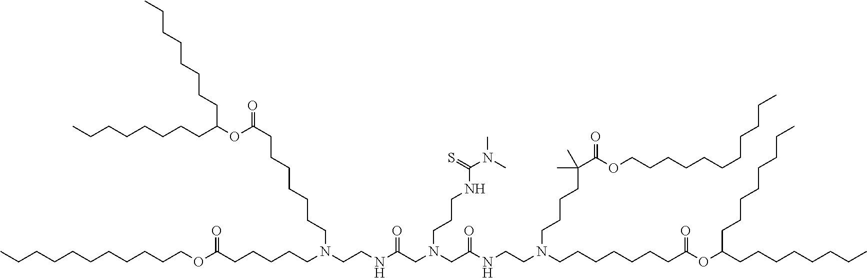 [SMILES: CCCCCCCCCCCOC(=O)CCCCCN(CCCCCCCC(=O)OC(CCCCCCCC)CCCCCCCC)CCNC(=O)CN(CCCNC(=S)N(C)C)CC(=O)NCCN(CCCCCCCC(=O)OC(CCCCCCCC)CCCCCCCC)CCCCC(C)(C)C(=O)OCCCCCCCCCCC] |
| A45 | 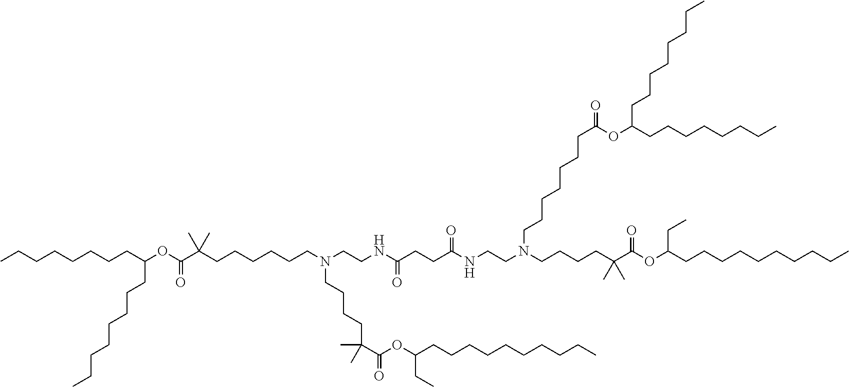 [SMILES: CCCCCCCCCCC(CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] |
| A46 | 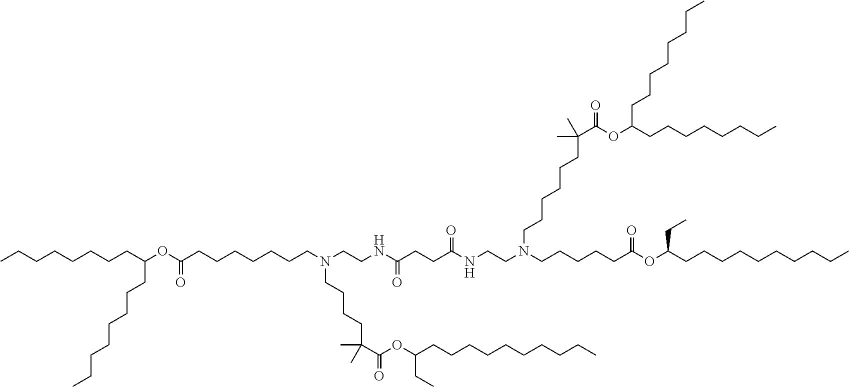 [SMILES: CCCCCCCCCCC(CC)OC(=O)C(C)(C)CCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)O[C@@H](CC)CCCCCCCCCC] |
| A47 | 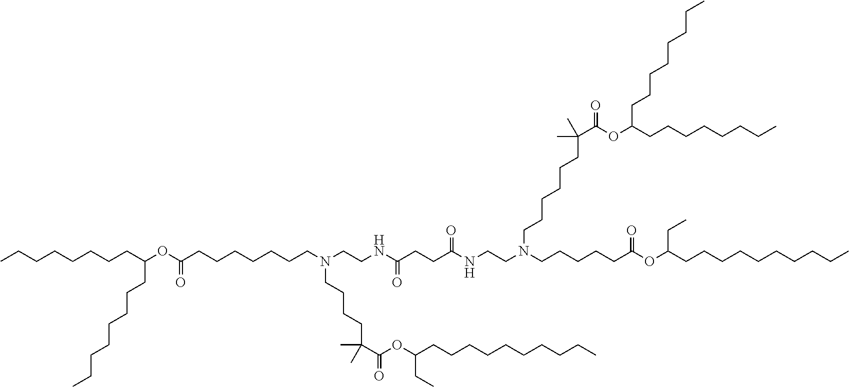 [SMILES: CCCCCCCCCCC(CC)OC(=O)CCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] |
| A48 | 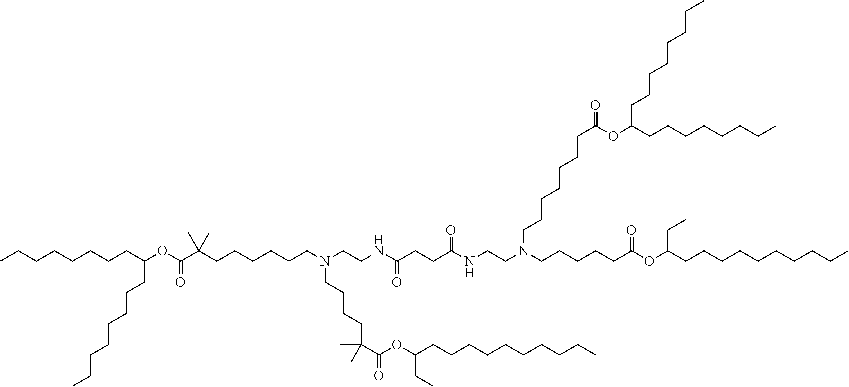 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCC(C)(C)C(=O)OC(CC)CCCCCCCCCC] |
| A49 | 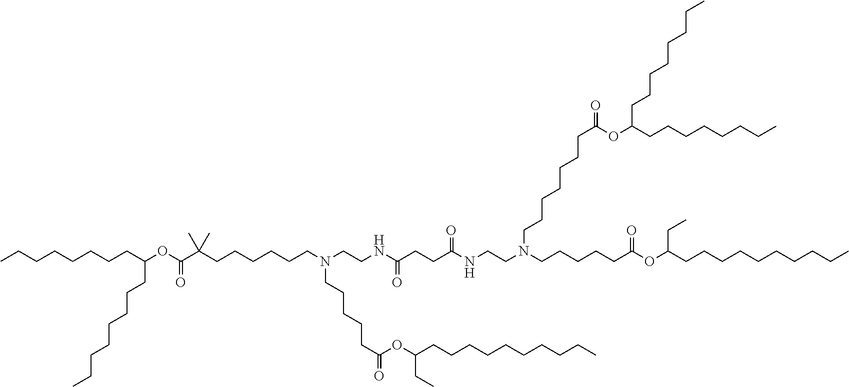 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCC(C)(C)C(=O)OC(CCCCCCCC)CCCCCCCC)CCCCCC(=O)OC(CC)CCCCCCCCCC] |
| A50 | 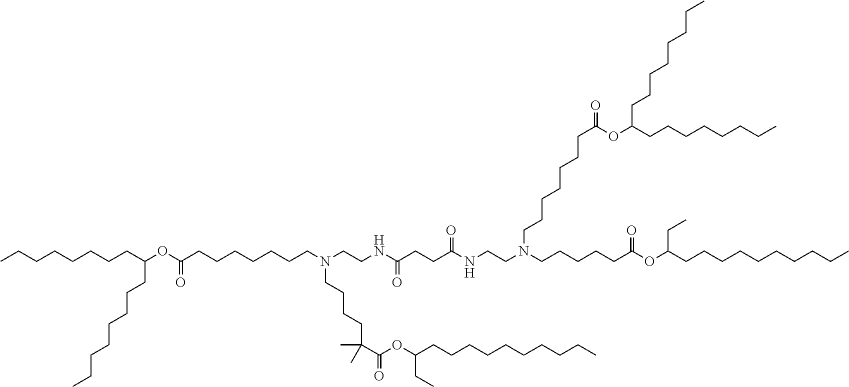 [SMILES: CCCCCCCCCCC(CC)OC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OC(CC)CCCCCCCCCC] |
| A51 | 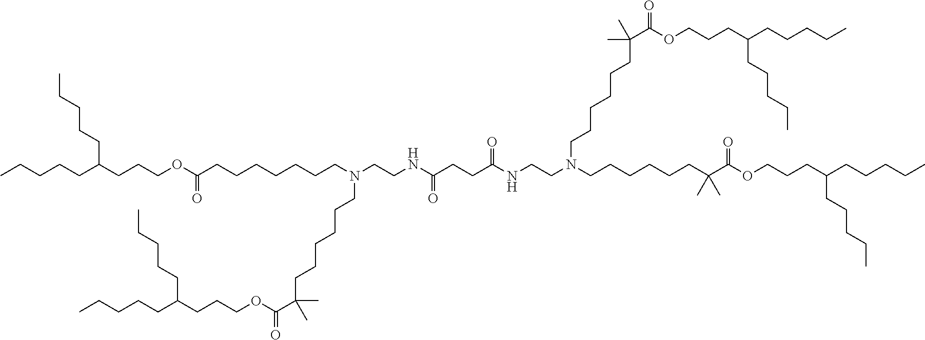 [SMILES: CCCCCC(CCCCC)CCCOC(=O)CCCCCCCN(CCCCCCC(C)(C)C(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] |
| A52 | 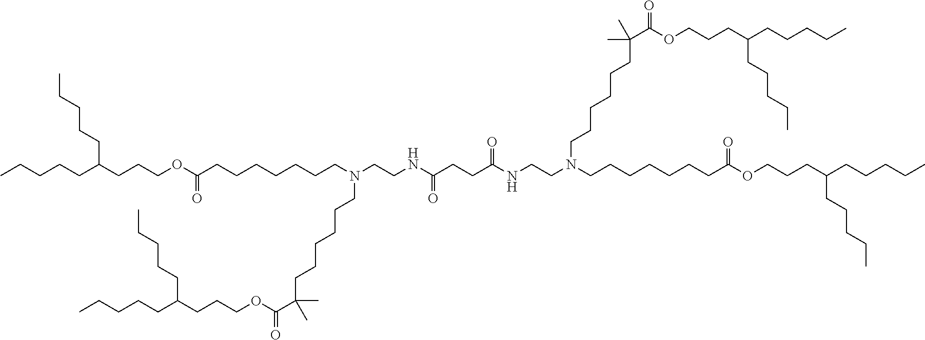 [SMILES: CCCCCC(CCCCC)CCCOC(=O)CCCCCCCN(CCCCCCC(C)(C)C(=O)OCCCC(CCCCC)CCCCC)CCNC(=O)CCC(=O)NCCN(CCCCCCCC(=O)OCCCC(CCCCC)CCCCC)CCCCCCC(C)(C)C(=O)OCCCC(CCCCC)CCCCC] |
| A53 | 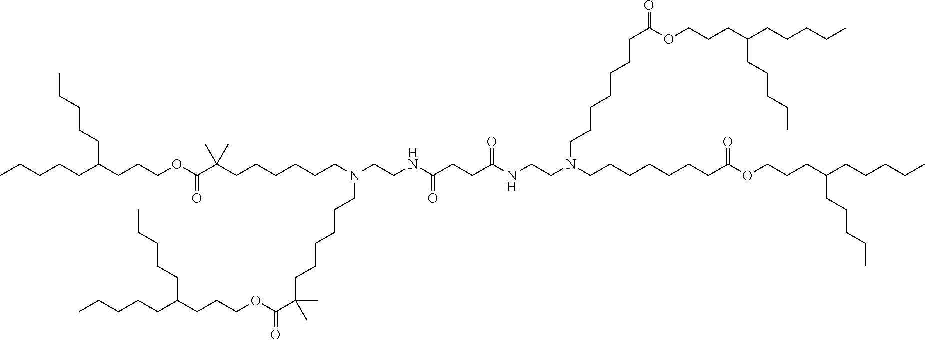 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCC(C)(C)C(=O)OCCCC(CCCCC)CCCCC)CCCCCCC(C)(C)C(=O)OCCCC(CCCCC)CCCCC] |
| A54 | 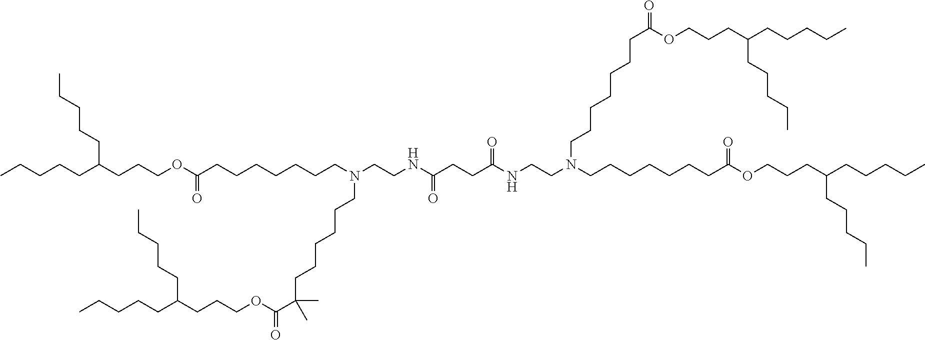 [SMILES: CCCCCC(CCCCC)CCCOC(=O)CCCCCCCN(CCCCCCCC(=O)OCCCC(CCCCC)CCCCC)CCNC(=O)CCC(=O)NCCN(CCCCCCCC(=O)OCCCC(CCCCC)CCCCC)CCCCCCC(C)(C)C(=O)OCCCC(CCCCC)CCCCC] |
| A55 | 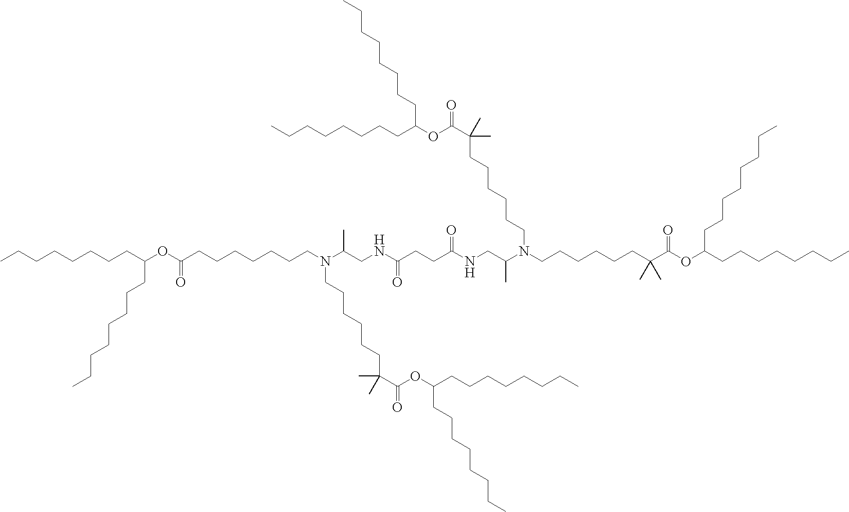 [SMILES: CCCCCCCCC(CCCCCCCC)OC(=O)CCCCCCCN(CCCCCCC(C)(C)C(=O)OC(CCCCCCCC)CCCCCCCC)C(C)CNC(=O)CCC(=O)NCC(C)N(CCCCCCC(C)(C)C(=O)OC(CCCCCCCC)CCCCCCCC)CCCCCCC(C)(C)C(=O)OC(CCCCCCCC)CCCCCCCC] |
| A56 | 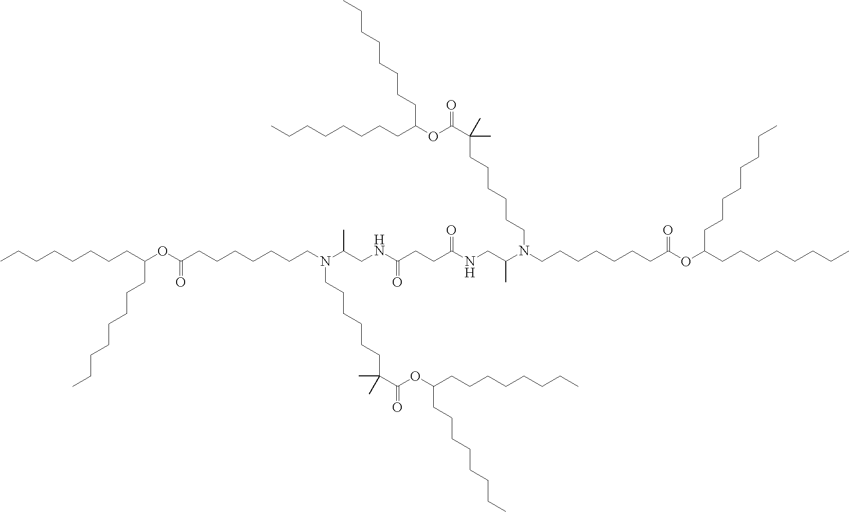 [SMILES: CCCCCCCCC(CCCCCCCC)OC(=O)CCCCCCCN(CCCCCCC(C)(C)C(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCC(C)(C)C(=O)OC(CCCCCCCC)CCCCCCCC] |
| A57 | 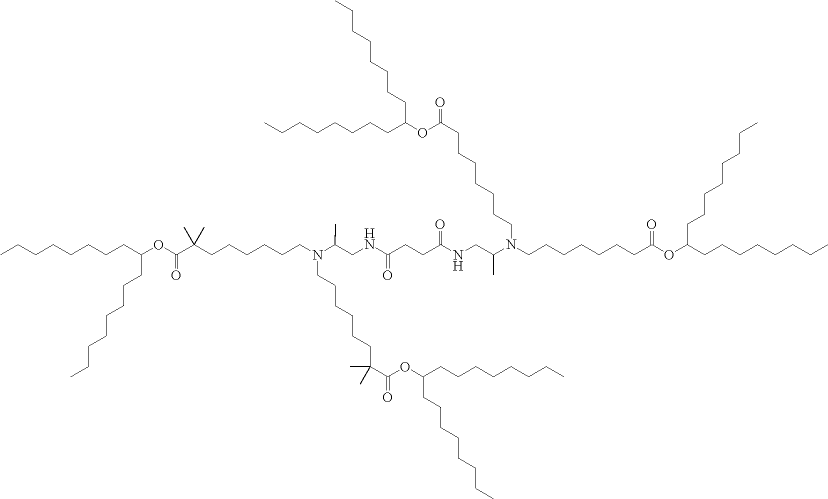 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCC(C)N(CCCCCCC(C)(C)C(=O)OC(CCCCCCCC)CCCCCCCC)CCCCCCC(C)(C)C(=O)OC(CCCCCCCC)CCCCCCCC] |
| A58 | 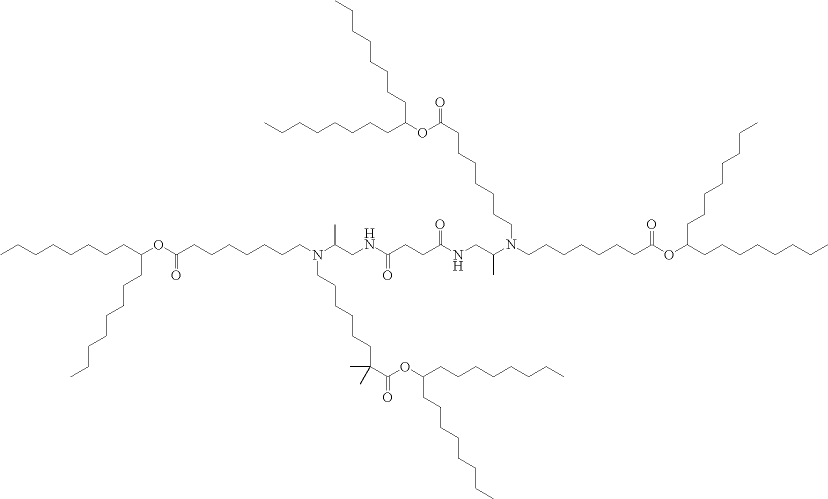 [SMILES: CCCCCCCCC(CCCCCCCC)OC(=O)CCCCCCCN(CCCCCCCC(=O)OC(CCCCCCCC)CCCCCCCC)C(C)CNC(=O)CCC(=O)NCC(C)N(CCCCCCCC(=O)OC(CCCCCCCC)CCCCCCCC)CCCCCCC(C)(C)C(=O)OC(CCCCCCCC)CCCCCCCC] |
| A59 | 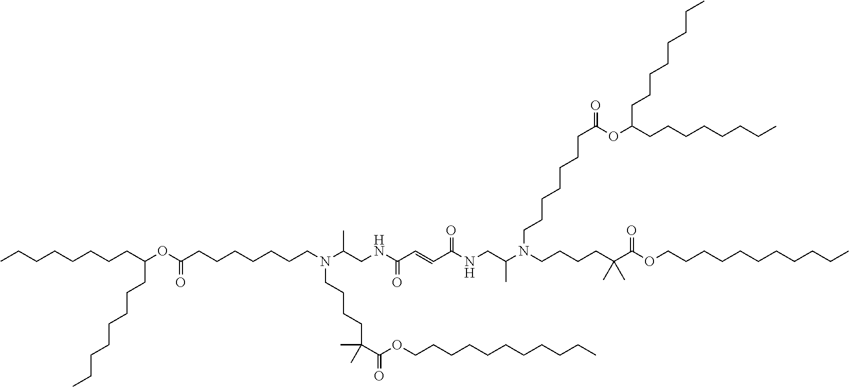 [SMILES: CCCCCCCCCCCOC(=O)C(C)(C)CCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] |
| A60 | 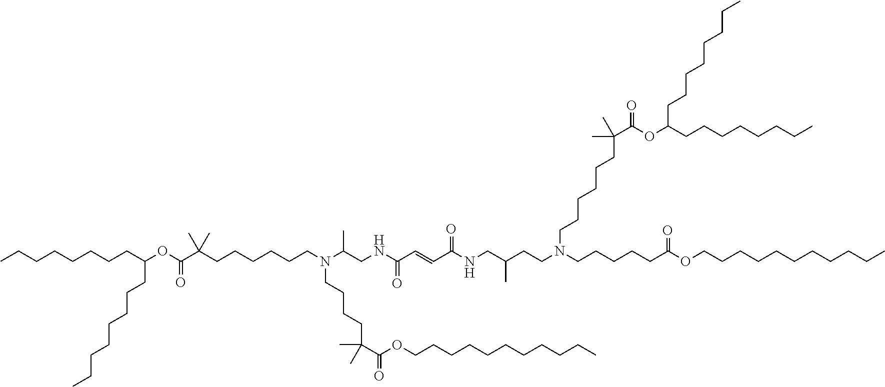 [SMILES: CCCCCCCCCCCOC(=O)CCCCCN(CCCCCCC(C)(C)C(=O)OC(CCCCCCCC)CCCCCCCC)CCC(C)CNC(=O)/C=C/C(=O)NCC(C)N(CCCCCCC(C)(C)C(=O)OC(CCCCCCCC)CCCCCCCC)CCCCC(C)(C)C(=O)OCCCCCCCCCCC] |
| A61 | 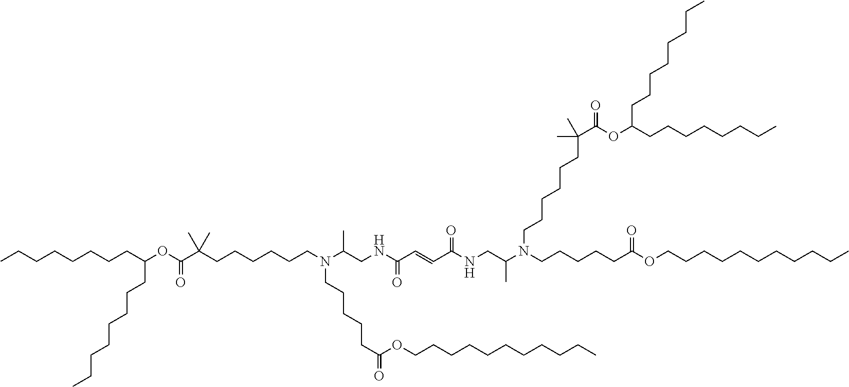 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCCC(=O)OCCCCCCCCCCC] |
| A62 | 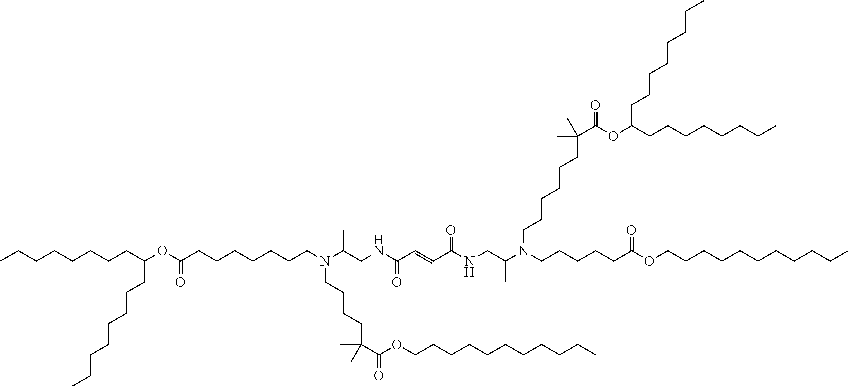 [SMILES: CCCCCCCCCCCOC(=O)CCCCCN(CCCCCCC(C)(C)C(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] |
| A63 | 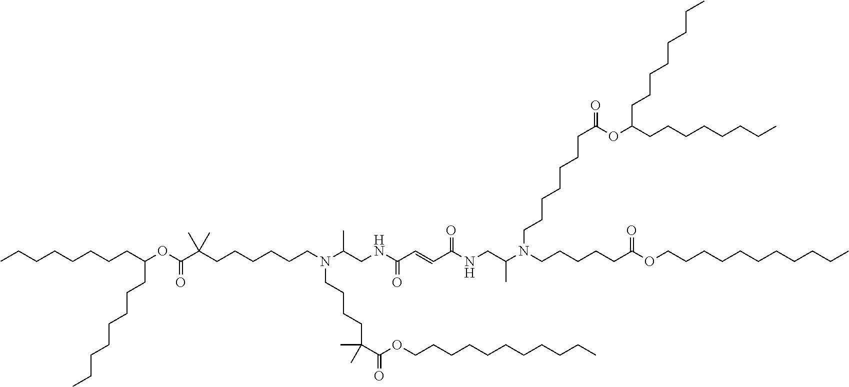 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCC(C)(C)C(=O)OCCCCCCCCCCC] |
| A64 | 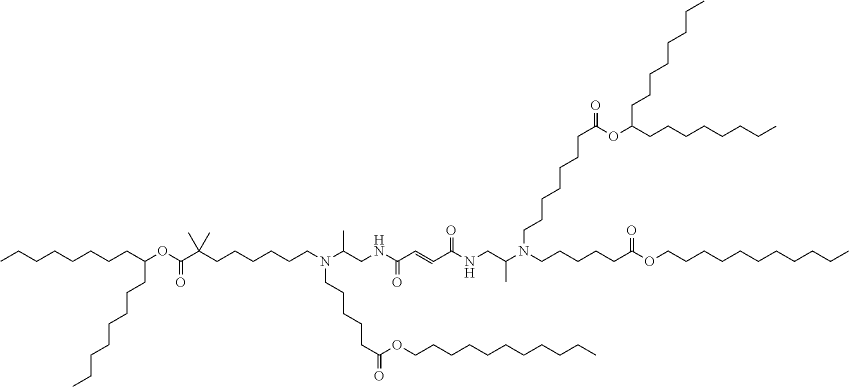 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCC(C)(C)C(=O)OC(CCCCCCCC)CCCCCCCC)CCCCCC(=O)OCCCCCCCCCCC] |
| A65 | 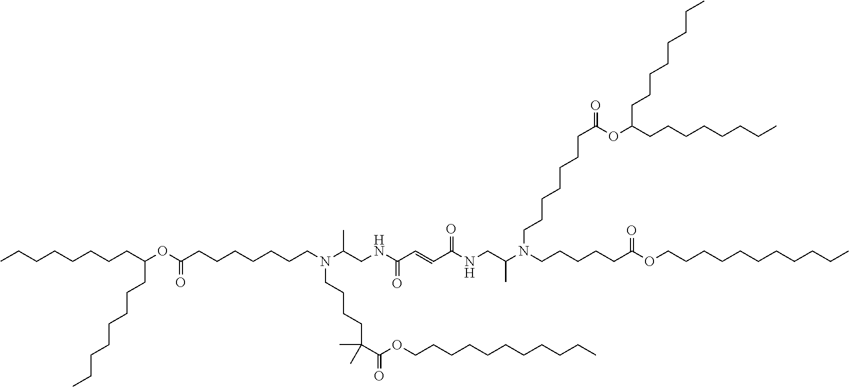 [SMILES: CCCCCCCCCCCOC(=O)CCCCCN(CCCCCCCC(=O)OC(CCCCCCCC)CCCCCCCC)C(C)CNC(=O)/C=C/C(=O)NCC(C)N(CCCCCCCC(=O)OC(CCCCCCCC)CCCCCCCC)CCCCC(C)(C)C(=O)OCCCCCCCCCCC] |
| A66 | 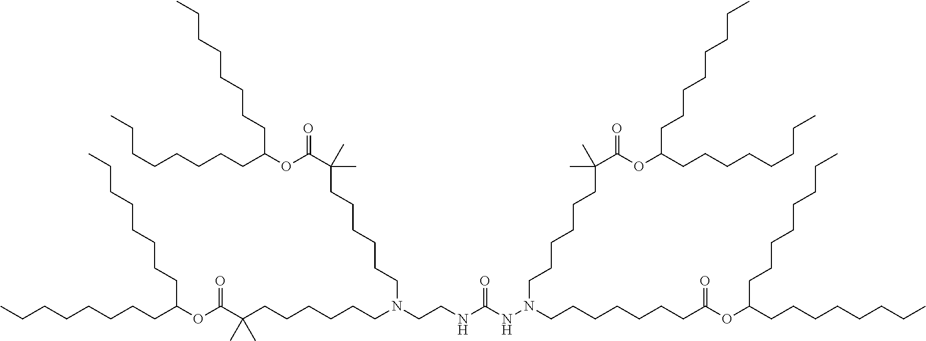 [SMILES: CCCCCCCCC(CCCCCCCC)OC(=O)CCCCCCCN(CCCCCCC(C)(C)C(=O)OC(CCCCCCCC)CCCCCCCC)NC(=O)NCCN(CCCCCCC(C)(C)C(=O)OC(CCCCCCCC)CCCCCCCC)CCCCCCC(C)(C)C(=O)OC(CCCCCCCC)CCCCCCCC] |
| A67 | 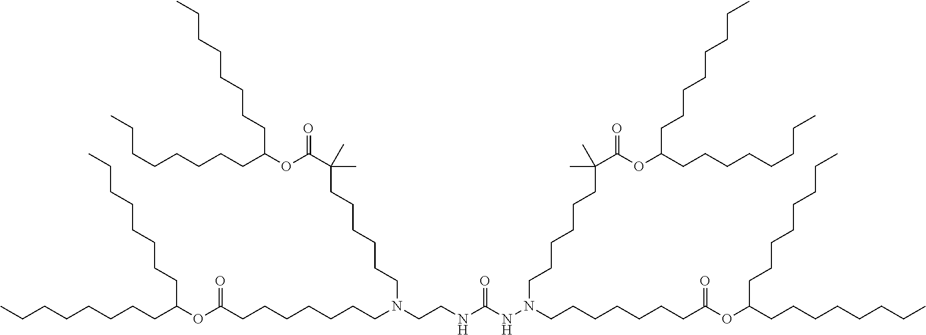 [SMILES: CCCCCCCCC(CCCCCCCC)OC(=O)CCCCCCCN(CCCCCCC(C)(C)C(=O)OC(CCCCCCCC)CCCCCCCC)CCNC(=O)NN(CCCCCCCC(=O)OC(CCCCCCCC)CCCCCCCC)CCCCCCC(C)(C)C(=O)OC(CCCCCCCC)CCCCCCCC] |
| A68 | 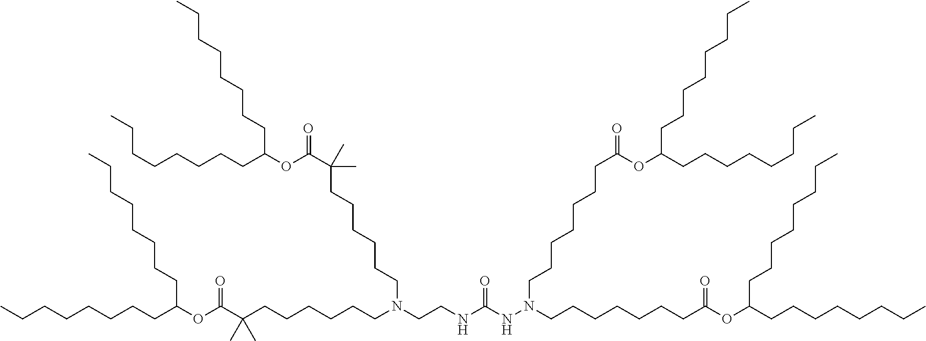 [SMILES: CCCCCCCCC(CCCCCCCC)OC(=O)CCCCCCCN(CCCCCCCC(=O)OC(CCCCCCCC)CCCCCCCC)NC(=O)NCCN(CCCCCCC(C)(C)C(=O)OC(CCCCCCCC)CCCCCCCC)CCCCCCC(C)(C)C(=O)OC(CCCCCCCC)CCCCCCCC] |
| A69 | 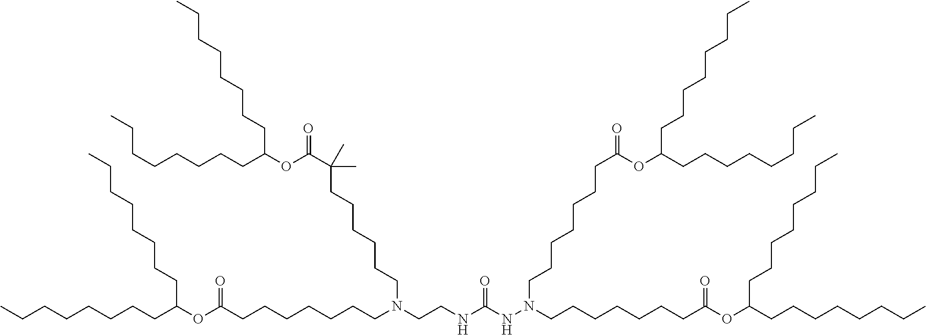 [SMILES: CCCCCCCCC(CCCCCCCC)OC(=O)CCCCCCCN(CCCCCCC(C)(C)C(=O)OC(CCCCCCCC)CCCCCCCC)CCNC(=O)NN(CCCCCCCC(=O)OC(CCCCCCCC)CCCCCCCC)CCCCCCCC(=O)OC(CCCCCCCC)CCCCCCCC] |
| A70 | 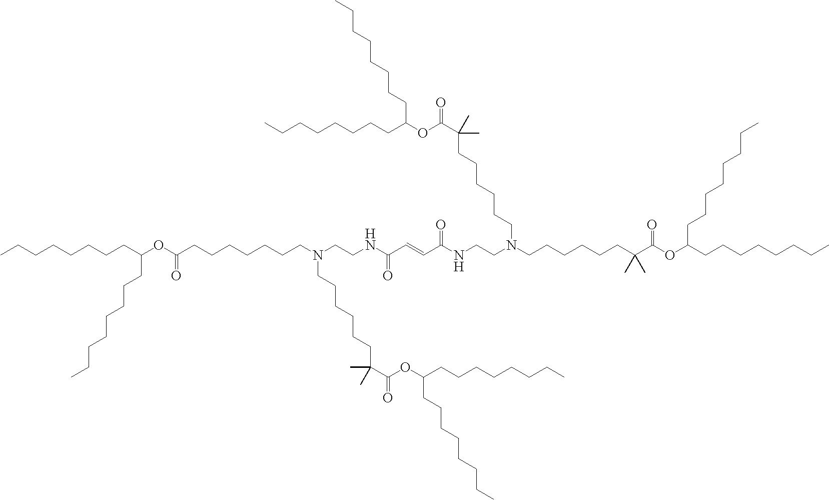 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCCC(C)(C)C(=O)OC(CCCCCCCC)CCCCCCCC] |
| A71 | 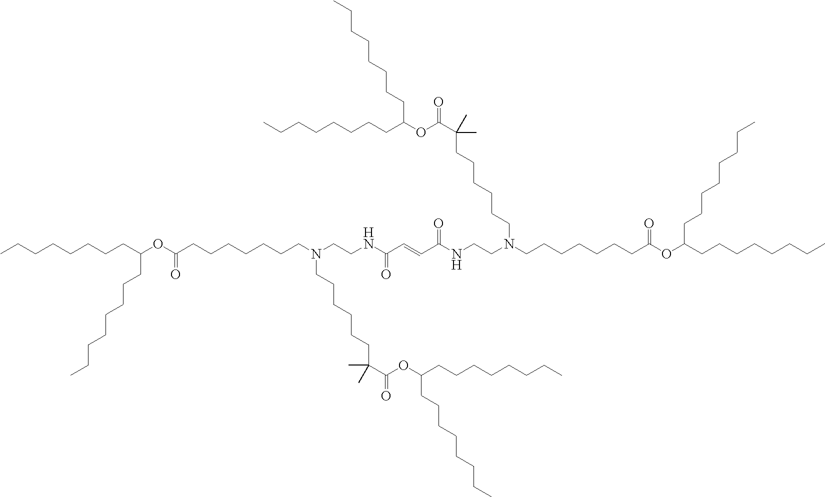 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCCCC(C)(C)C(=O)OC(CCCCCCCC)CCCCCCCC] |
| A72 | 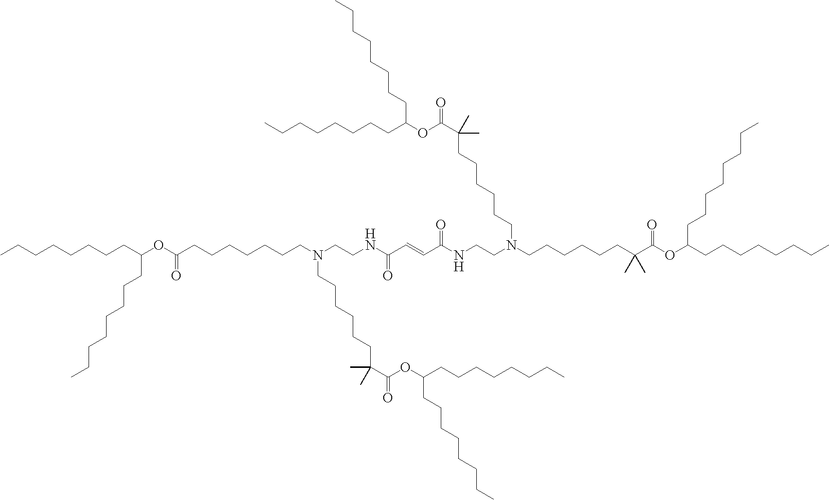 [SMILES: CCCCCCCCC(CCCCCCCC)OC(=O)CCCCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCCC(C)(C)C(=O)OC(CCCCCCCC)CCCCCCCC] |
| A73 | 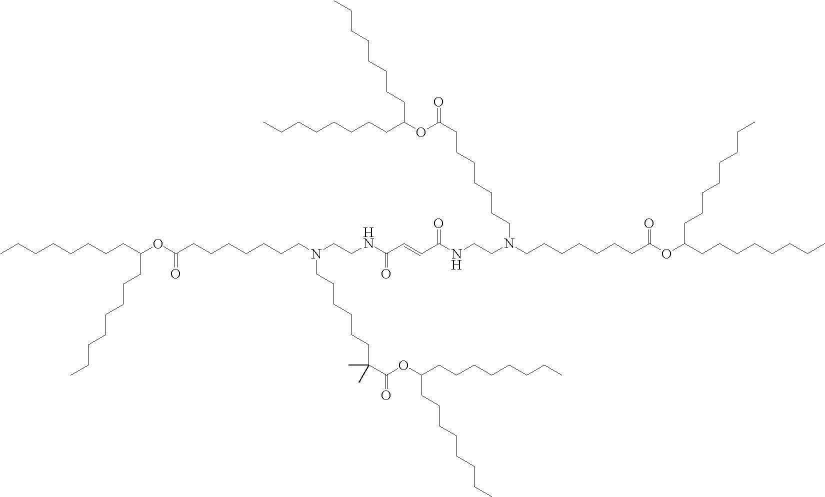 [SMILES: CCCCCCCCC(CCCCCCCC)OC(=O)CCCCCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCCCC(C)(C)C(=O)OC(CCCCCCCC)CCCCCCCC] |
| A74 | 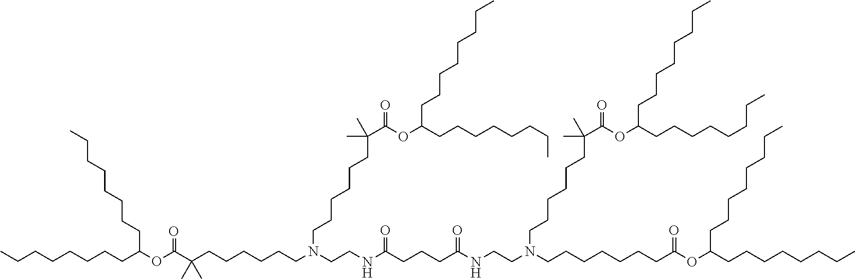 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCCC(=O)NCCN(CCCCCCC(C)(C)C(=O)OC(CCCCCCCC)CCCCCCCC)CCCCCCC(C)(C)C(=O)OC(CCCCCCCC)CCCCCCCC] |
| A75 | 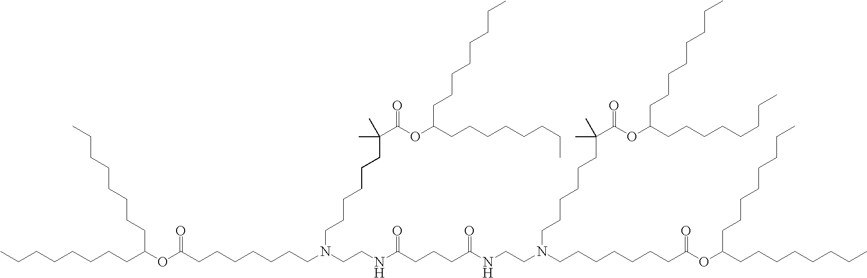 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCCC(=O)NCCN(CCCCCCCC(=O)OC(CCCCCCCC)CCCCCCCC)CCCCCCC(C)(C)C(=O)OC(CCCCCCCC)CCCCCCCC] |
| A76 | 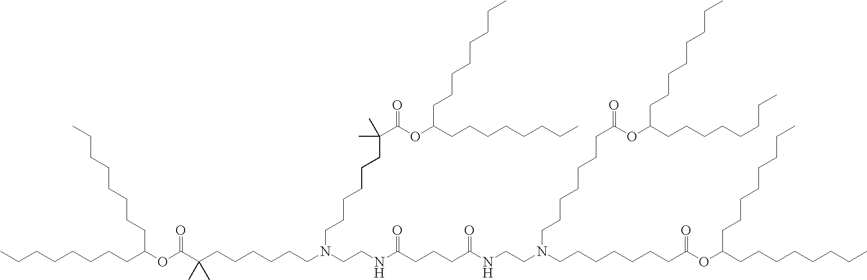 [SMILES: CCCCCCCCC(CCCCCCCC)OC(=O)CCCCCCCN(CCCCCCCC(=O)OC(CCCCCCCC)CCCCCCCC)CCNC(=O)CCCC(=O)NCCN(CCCCCCC(C)(C)C(=O)OC(CCCCCCCC)CCCCCCCC)CCCCCCC(C)(C)C(=O)OC(CCCCCCCC)CCCCCCCC] |
| A77 | 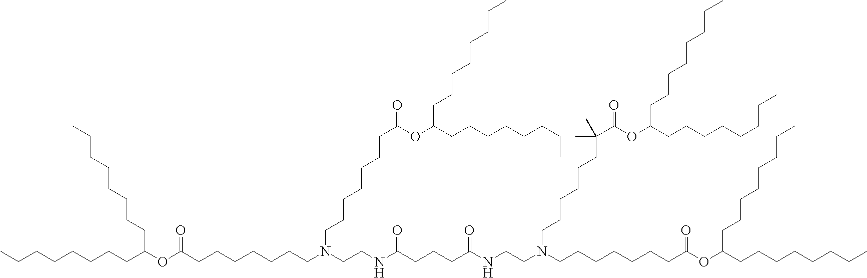 [SMILES: CCCCCCCCC(CCCCCCCC)OC(=O)CCCCCCCN(CCCCCCCC(=O)OC(CCCCCCCC)CCCCCCCC)CCNC(=O)CCCC(=O)NCCN(CCCCCCCC(=O)OC(CCCCCCCC)CCCCCCCC)CCCCCCC(C)(C)C(=O)OC(CCCCCCCC)CCCCCCCC] |
| A78 | 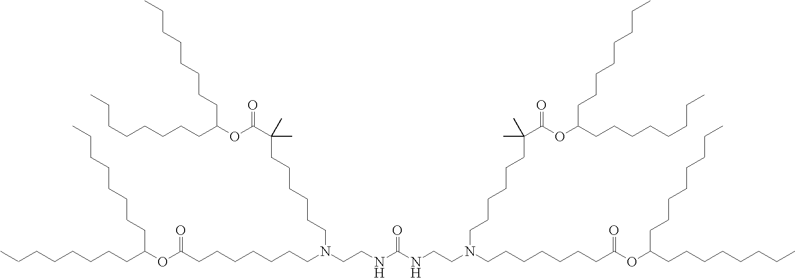 [SMILES: CCCCCCCCC(CCCCCCCC)OC(=O)CCCCCCCN(CCCCCCC(C)(C)C(=O)OC(CCCCCCCC)CCCCCCCC)CCNC(=O)NCCN(CCCCCCCC(=O)OC(CCCCCCCC)CCCCCCCC)CCCCCCC(C)(C)C(=O)OC(CCCCCCCC)CCCCCCCC] |
| A79 | 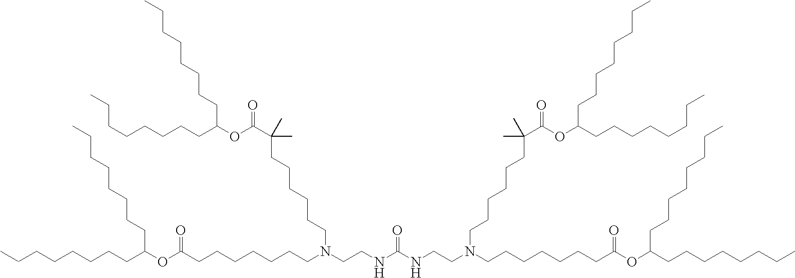 [SMILES: CCCCCCCCC(CCCCCCCC)OC(=O)CCCCCCCN(CCCCCCC(C)(C)C(=O)OC(CCCCCCCC)CCCCCCCC)CCNC(=O)NCCN(CCCCCCCC(=O)OC(CCCCCCCC)CCCCCCCC)CCCCCCC(C)(C)C(=O)OC(CCCCCCCC)CCCCCCCC] |
| A80 | 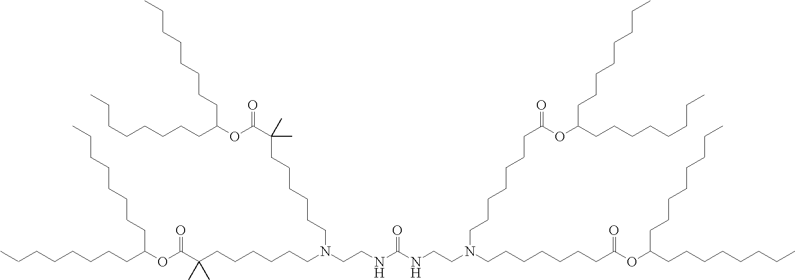 [SMILES: CCCCCCCCC(CCCCCCCC)OC(=O)CCCCCCCN(CCCCCCCC(=O)OC(CCCCCCCC)CCCCCCCC)CCNC(=O)NCCN(CCCCCCC(C)(C)C(=O)OC(CCCCCCCC)CCCCCCCC)CCCCCCC(C)(C)C(=O)OC(CCCCCCCC)CCCCCCCC] |
| A81 | 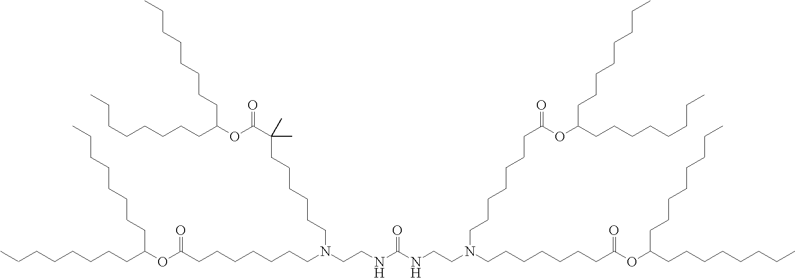 [SMILES: CCCCCCCCC(CCCCCCCC)OC(=O)CCCCCCCN(CCCCCCCC(=O)OC(CCCCCCCC)CCCCCCCC)CCNC(=O)NCCN(CCCCCCCC(=O)OC(CCCCCCCC)CCCCCCCC)CCCCCCC(C)(C)C(=O)OC(CCCCCCCC)CCCCCCCC] |
| A82 | 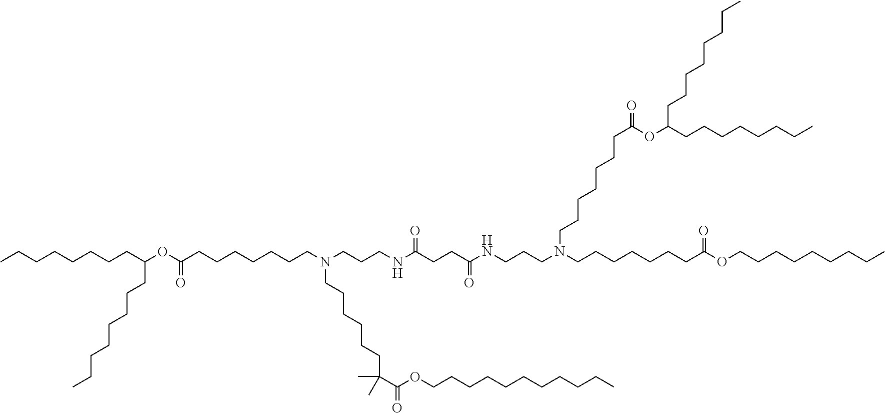 [SMILES: CCCCCCCCCCCOC(=O)C(C)(C)CCCCCCN(CCCCCCCC(=O)OC(CCCCCCCC)CCCCCCCC)CCCNC(=O)CCC(=O)NCCCN(CCCCCCCC(=O)OCCCCCCCCC)CCCCCCCC(=O)OC(CCCCCCCC)CCCCCCCC] |
| A83 | 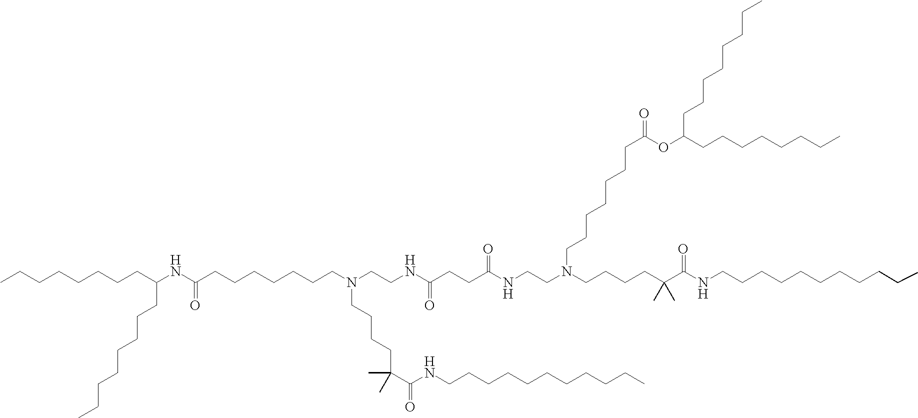 [SMILES: CCCCCCCCCCCNC(=O)C(C)(C)CCCCN(CCCCCCCC(=O)NC(CCCCCCCC)CCCCCCCC)CCNC(=O)CCC(=O)NCCN(CCCCCCCC(=O)OC(CCCCCCCC)CCCCCCCC)CCCCC(C)(C)C(=O)NCCCCCCCCCCC] |
| A84 | 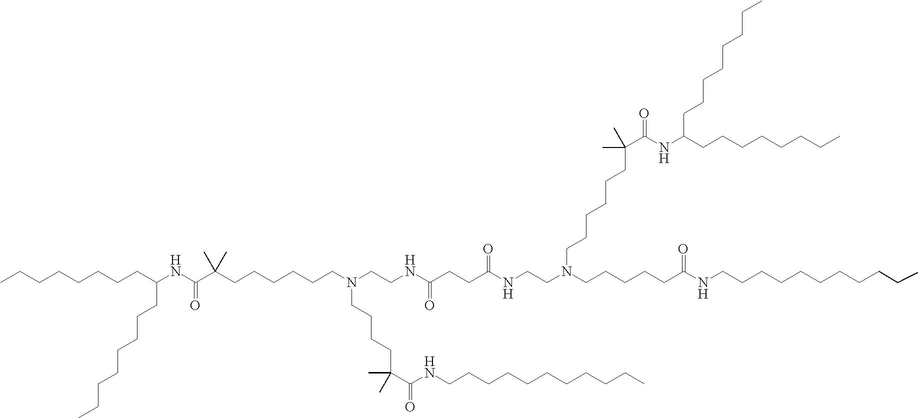 [SMILES: CCCCCCCCCCCNC(=O)CCCCCN(CCCCCCC(C)(C)C(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] |
| A85 | 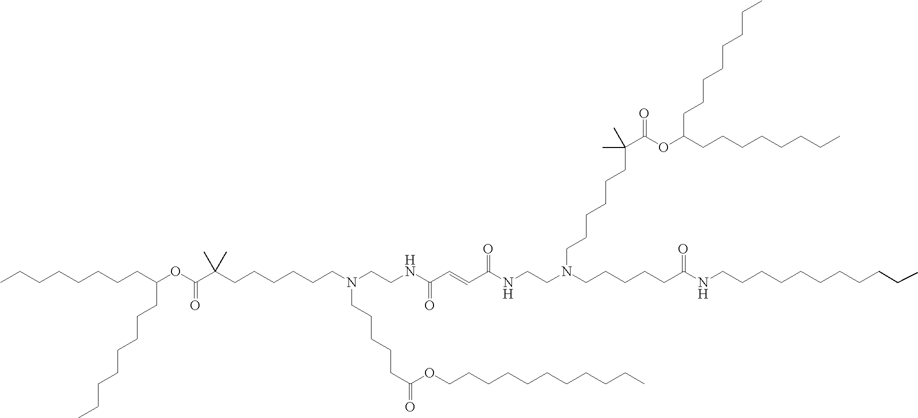 [SMILES: CCCCCCCCCCCNC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] |
| A86 | 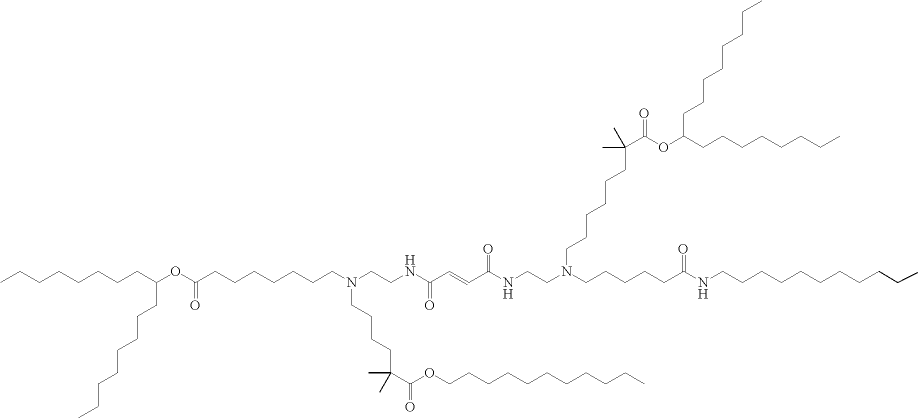 [SMILES: CCCCCCCCCCCNC(=O)CCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] |
| A87 | 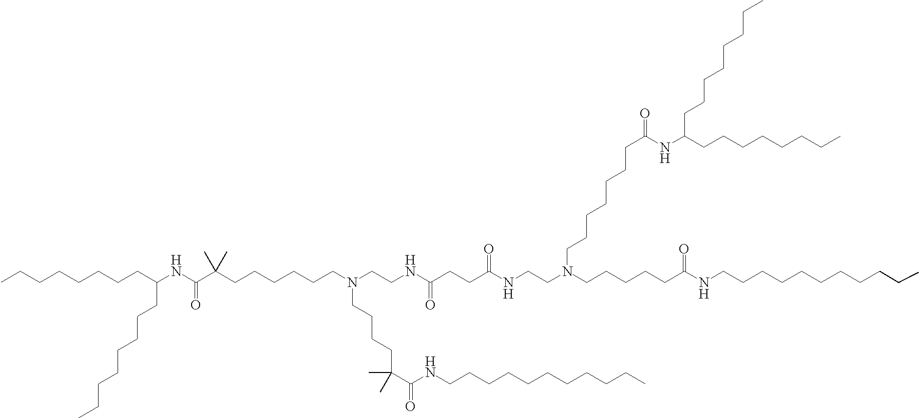 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCC(C)(C)C(=O)NC(CCCCCCCC)CCCCCCCC)CCCCC(C)(C)C(=O)NCCCCCCCCCCC] |
| A88 | 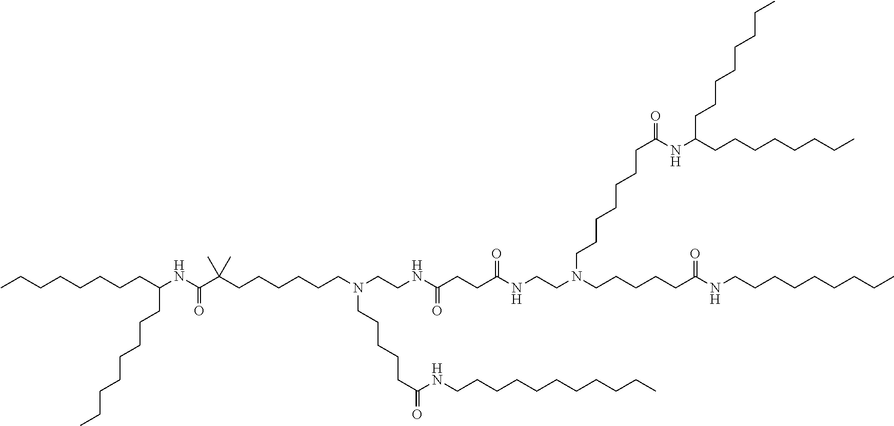 [SMILES: CCCCCCCCCCCNC(=O)CCCCCN(CCCCCCC(C)(C)C(=O)NC(CCCCCCCC)CCCCCCCC)CCNC(=O)CCC(=O)NCCN(CCCCCCCC(=O)NC(CCCCCCCC)CCCCCCCC)CCCCCC(=O)NCCCCCCCCC] |
| A89 | 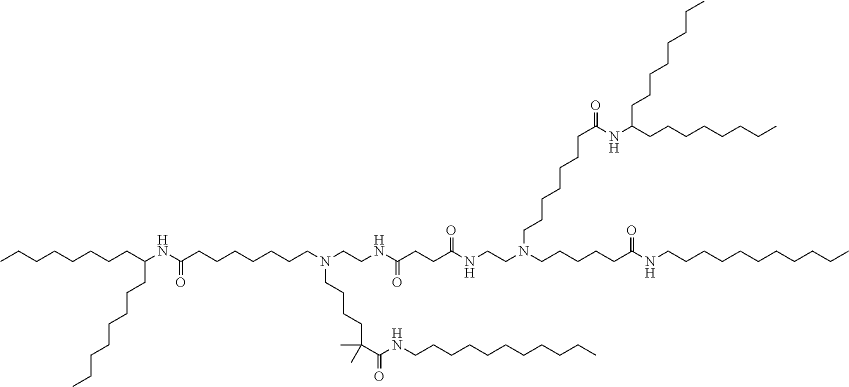 [SMILES: CCCCCCCCCCCNC(=O)CCCCCN(CCCCCCCC(=O)NC(CCCCCCCC)CCCCCCCC)CCNC(=O)CCC(=O)NCCN(CCCCCCCC(=O)NC(CCCCCCCC)CCCCCCCC)CCCCC(C)(C)C(=O)NCCCCCCCCCCC] |
| A90 | 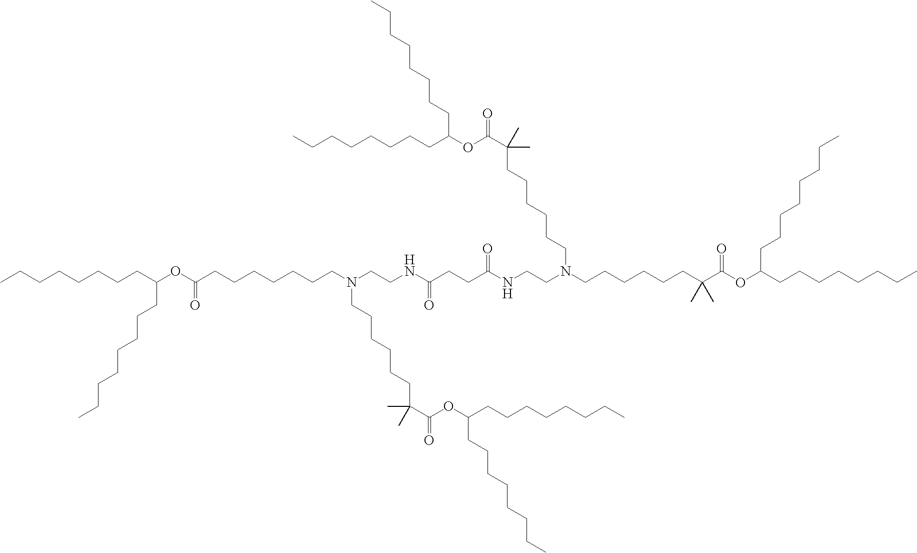 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCC(=O)NCCN(CCCCCCC(C)(C)C(=O)OC(CCCCCCCC)CCCCCCCC)CCCCCCC(C)(C)C(=O)OC(CCCCCCCC)CCCCCCCC] |
| A91 | 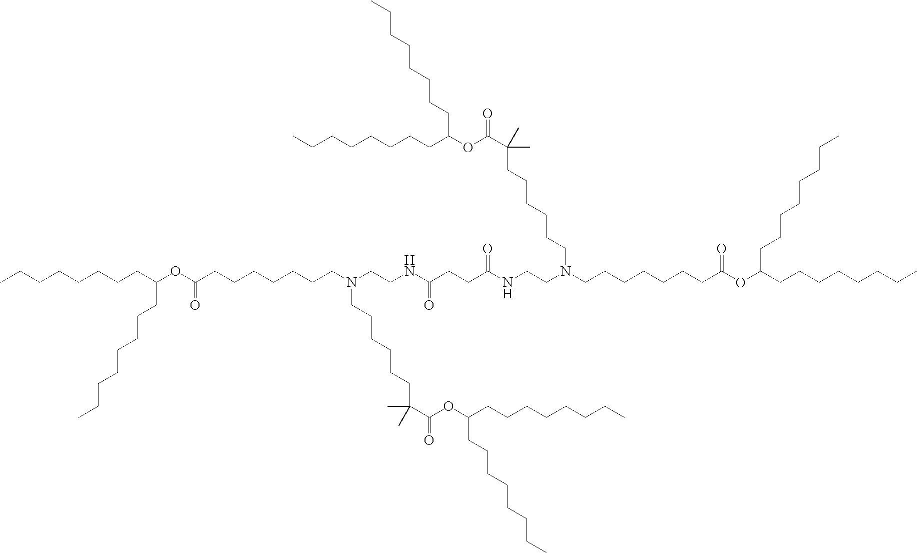 [SMILES: CCCCCCCCC(CCCCCCCC)OC(=O)CCCCCCCN(CCCCCCC(C)(C)C(=O)OC(CCCCCCCC)CCCCCCCC)CCNC(=O)CCC(=O)NCCN(CCCCCCCC(=O)OC(CCCCCCCC)CCCCCCCC)CCCCCCC(C)(C)C(=O)OC(CCCCCCCC)CCCCCCCC] |
| A92 | 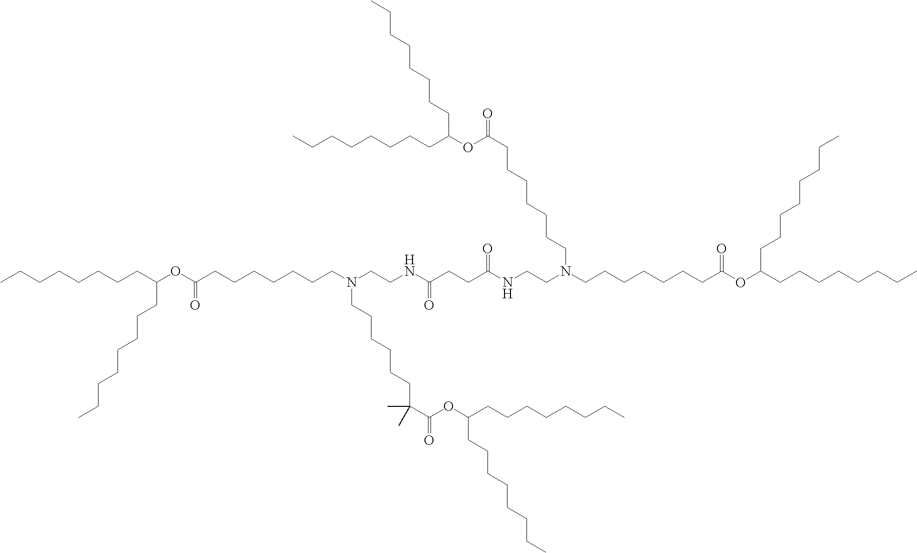 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCCC(C)(C)C(=O)OC(CCCCCCCC)CCCCCCCC] |
| A93 | 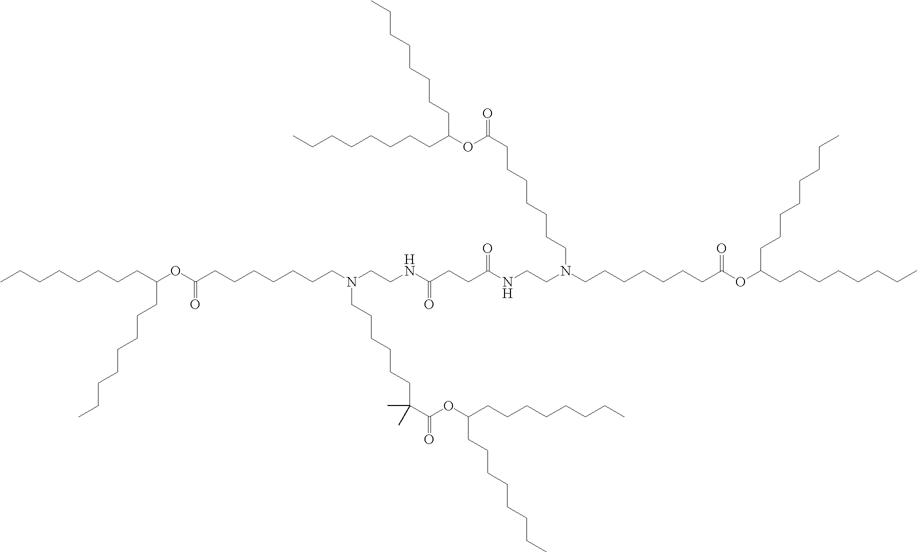 [SMILES: CCCCCCCCC(CCCCCCCC)OC(=O)CCCCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCCCC(C)(C)C(=O)OC(CCCCCCCC)CCCCCCCC] |
| A94 | 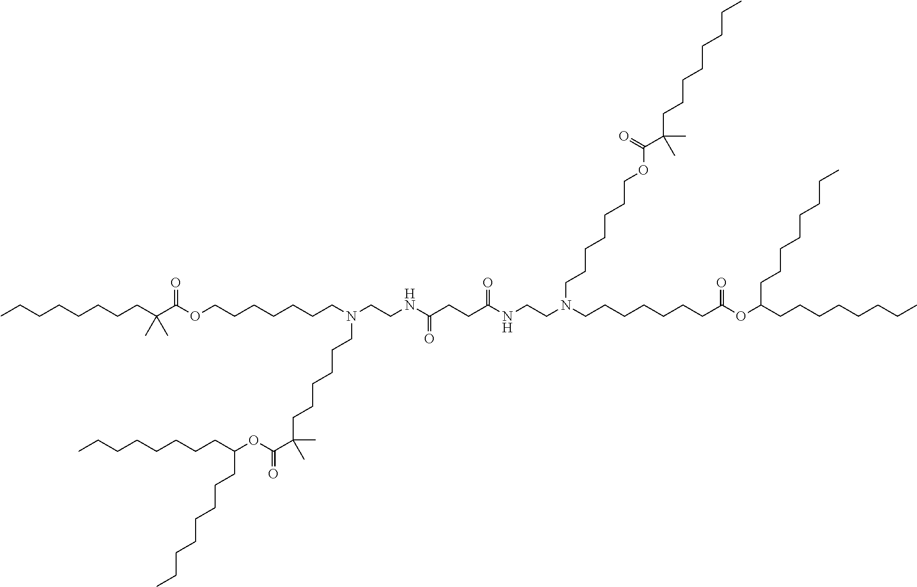 [SMILES: CCCCCCCCC(CCCCCCCC)OC(=O)CCCCCCCN(CCCCCCCOC(=O)C(C)(C)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] |
| A95 | 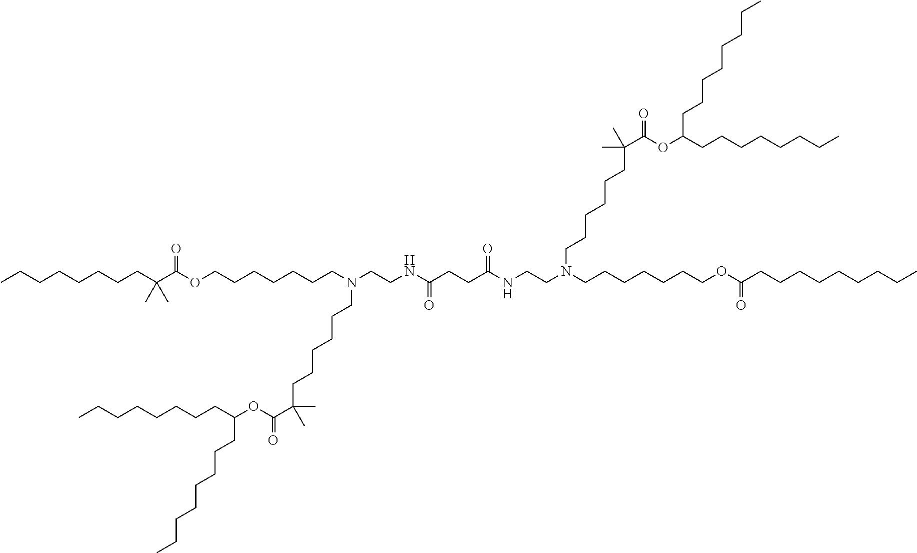 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)C(C)(C)CCCCCCCC)CCCCCCC(C)(C)C(=O)OC(CCCCCCCC)CCCCCCCC] |
| A96 | 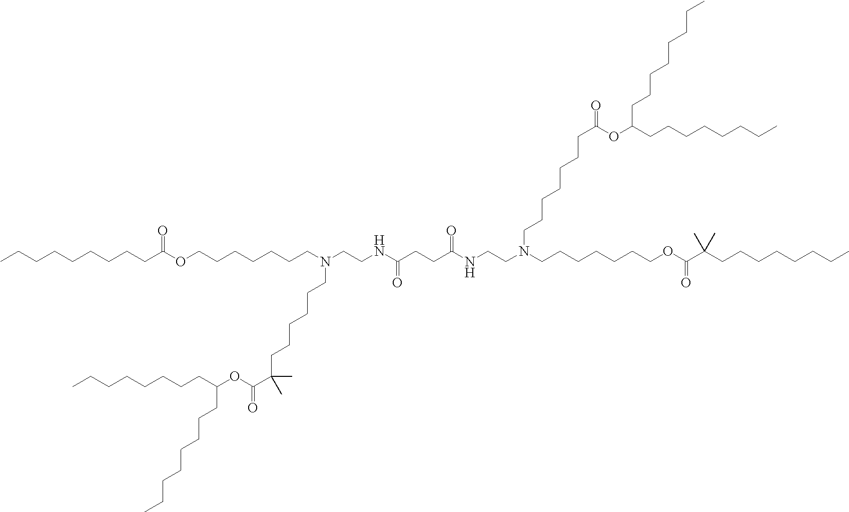 [SMILES: CCCCCCCCCC(=O)OCCCCCCCN(CCCCCCC(C)(C)C(=O)OC(CCCCCCCC)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] |
| A97 | 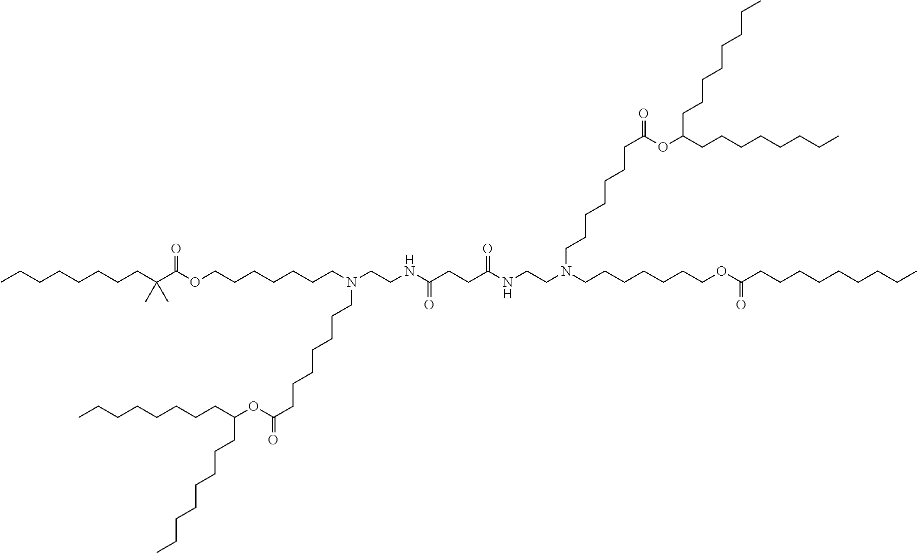 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)C(C)(C)CCCCCCCC)CCCCCCCC(=O)OC(CCCCCCCC)CCCCCCCC] |
| A98 | 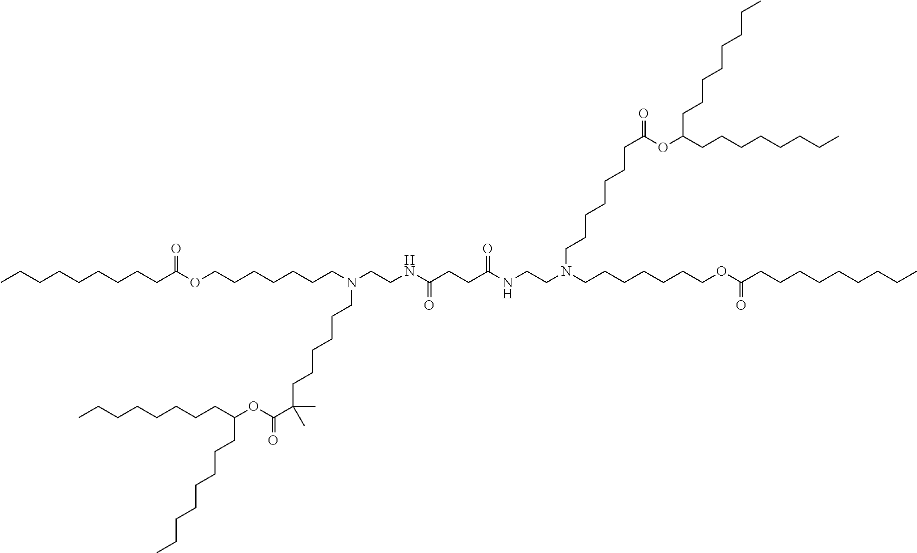 [SMILES: CCCCCCCCCC(=O)OCCCCCCCN(CCCCCCCC(=O)OC(CCCCCCCC)CCCCCCCC)CCNC(=O)CCC(=O)NCCN(CCCCCCCOC(=O)CCCCCCCCC)CCCCCCC(C)(C)C(=O)OC(CCCCCCCC)CCCCCCCC] |
| A99 | 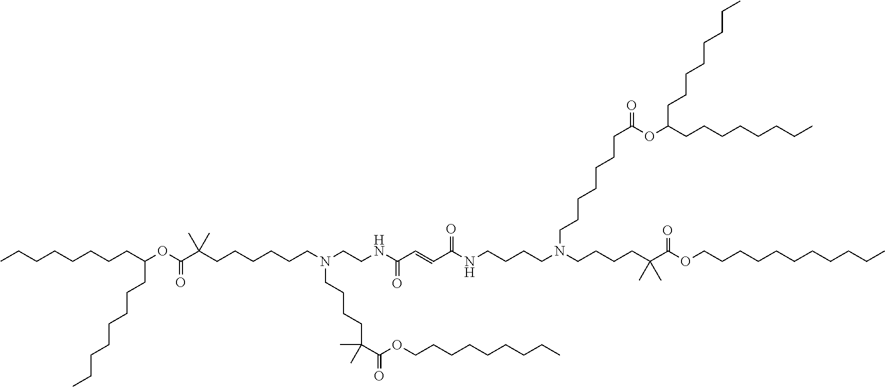 [SMILES: CCCCCCCCCCCOC(=O)C(C)(C)CCCCN(CCCCCCCC(=O)OC(CCCCCCCC)CCCCCCCC)CCCCNC(=O)/C=C/C(=O)NCCN(CCCCCCC(C)(C)C(=O)OC(CCCCCCCC)CCCCCCCC)CCCCC(C)(C)C(=O)OCCCCCCCCC] |
| A100 | 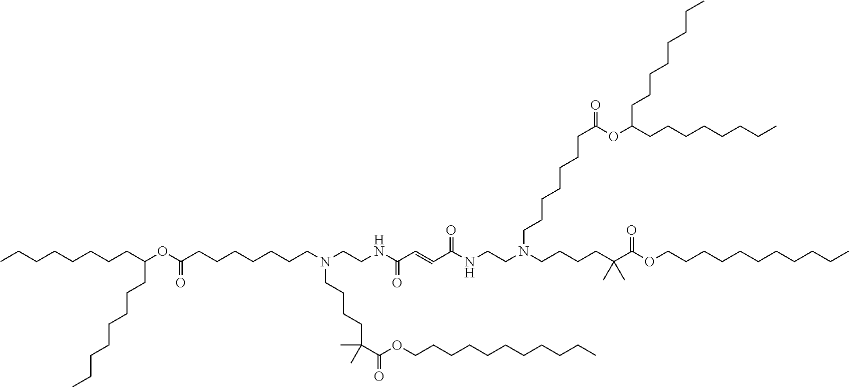 [SMILES: CCCCCCCCCCCOC(=O)C(C)(C)CCCCN(CCCCCCCC(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] |
| A101 | 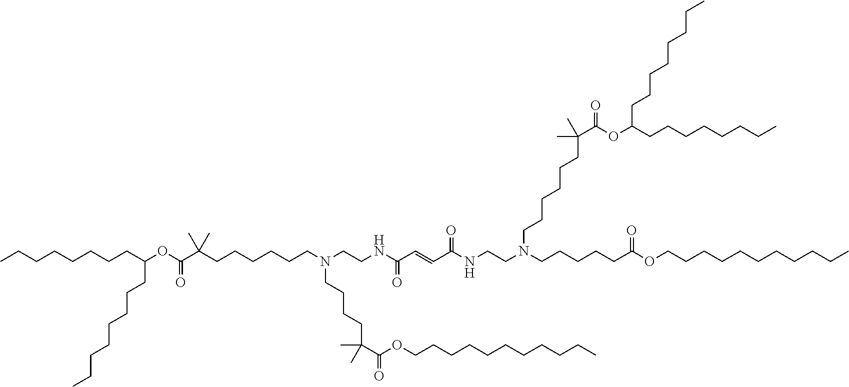 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] |
| A102 | 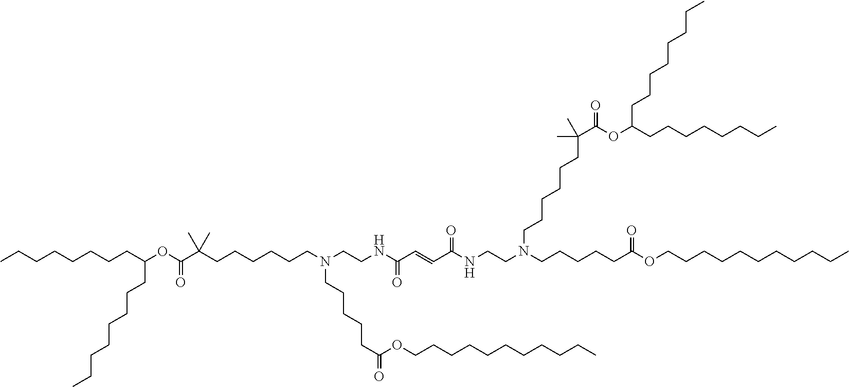 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] |
| A103 | 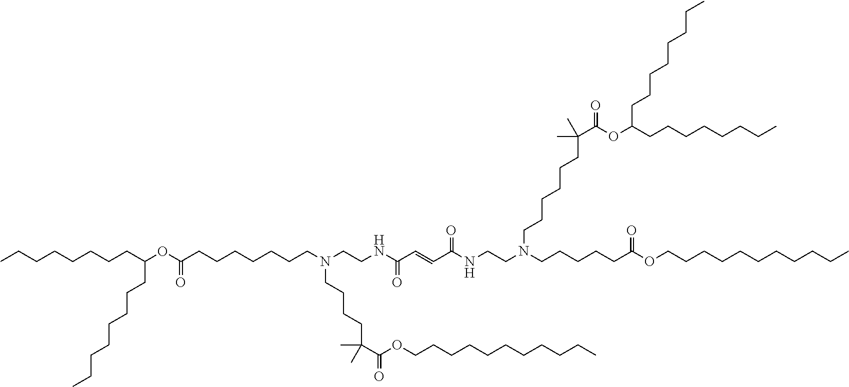 [SMILES: CCCCCCCCCCCOC(=O)CCCCCN(CCCCCCC(C)(C)C(=O)OC(CCCCCCCC)CCCCCCCC)CCNC(=O)/C=C/C(=O)NCCN(CCCCCCCC(=O)OC(CCCCCCCC)CCCCCCCC)CCCCC(C)(C)C(=O)OCCCCCCCCCCC] |
| A104 | 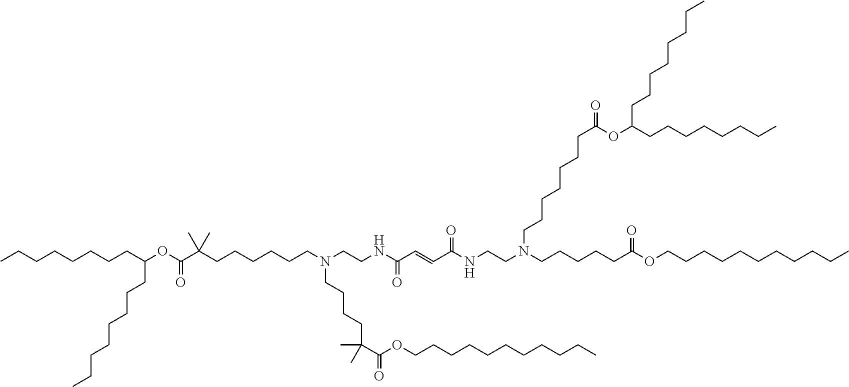 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCC(C)(C)C(=O)OCCCCCCCCCCC] |
| A105 | 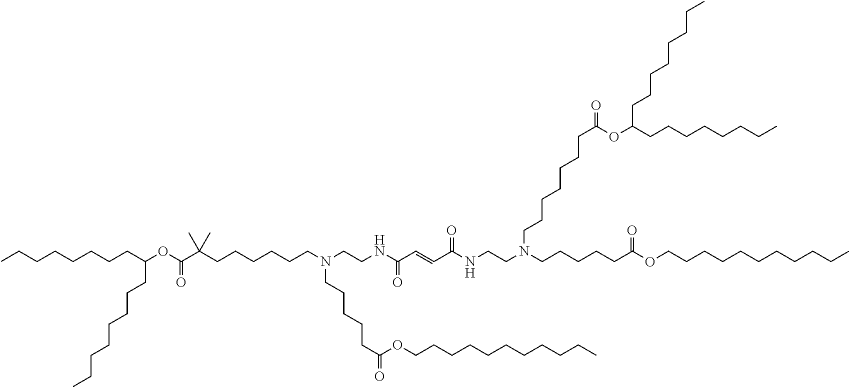 [SMILES: CCCCCCCCCCCOC(=O)CCCCCN(CCCCCCCC(=O)OC(CCCCCCCC)CCCCCCCC)CCNC(=O)/C=C/C(=O)NCCN(CCCCCCC(C)(C)C(=O)OC(CCCCCCCC)CCCCCCCC)CCCCCC(=O)OCCCCCCCCCCC] |
| A106 | 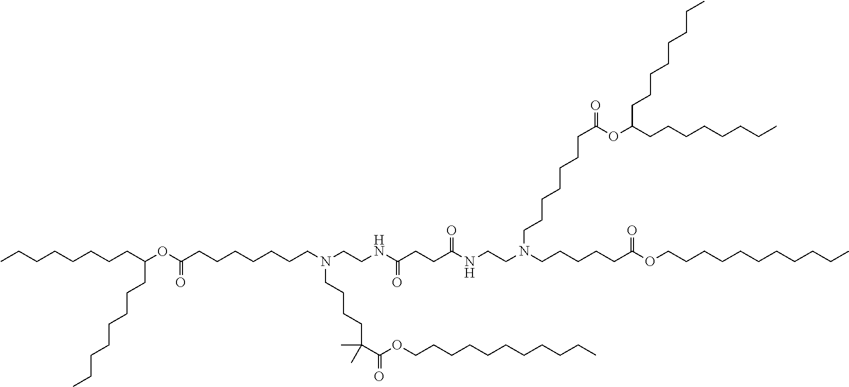 [SMILES: CCCCCCCCCCCOC(=O)CCCCCN(CCCCCCCC(=O)OC(CCCCCCCC)CCCCCCCC)CCNC(=O)CCC(=O)NCCN(CCCCCCCC(=O)OC(CCCCCCCC)CCCCCCCC)CCCCC(C)(C)C(=O)OCCCCCCCCCCC] |
| B1 | 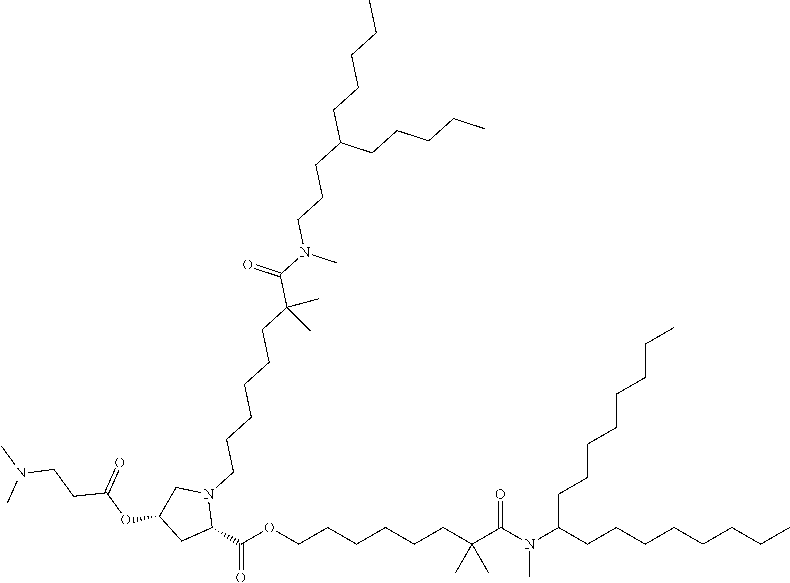 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCC(C)(C)C(=O)N(C)CCCC(CCCCC)CCCCC] |
| B2 | 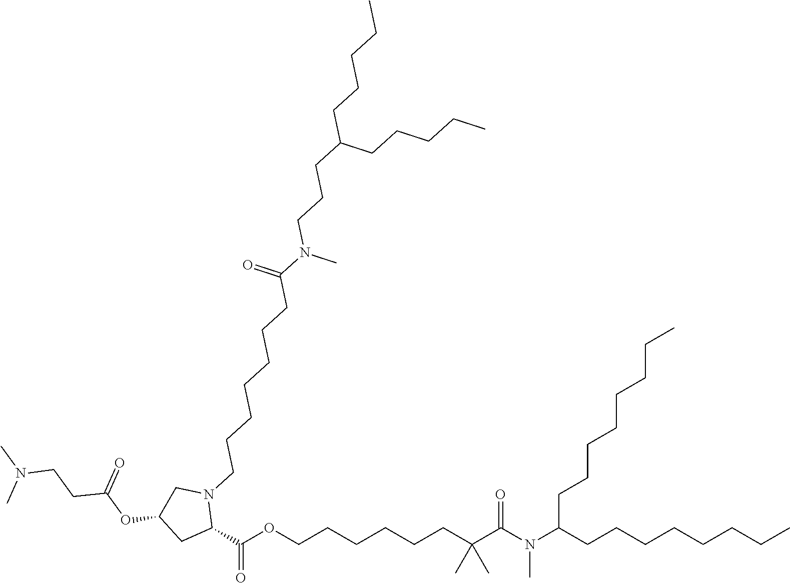 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCCC(=O)N(C)CCCC(CCCCC)CCCCC] |
| B3 | 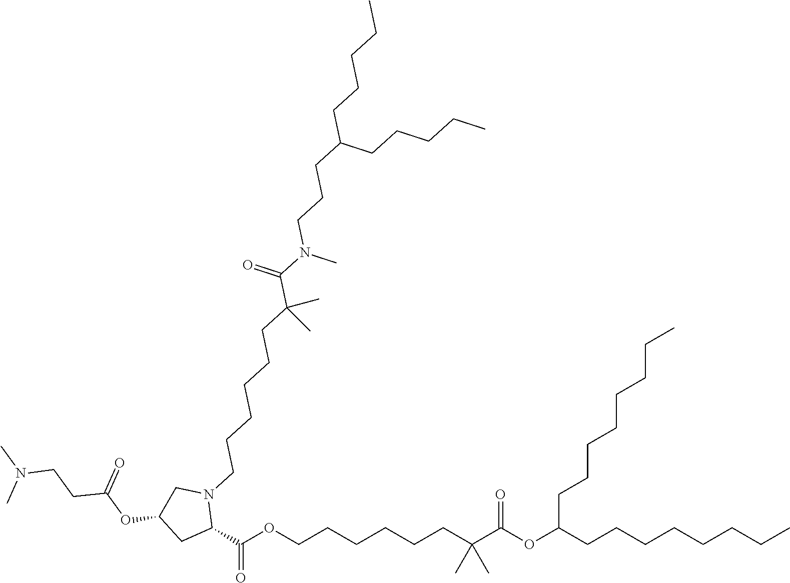 [SMILES: CCCCCCCCC(CCCCCCCC)OC(=O)C(C)(C)CCCCCCOC(=O)[C@@H]1C[C@H](OC(=O)CCN(C)C)CN1CCCCCCC(C)(C)C(=O)N(C)CCCC(CCCCC)CCCCC] |
| B4 | 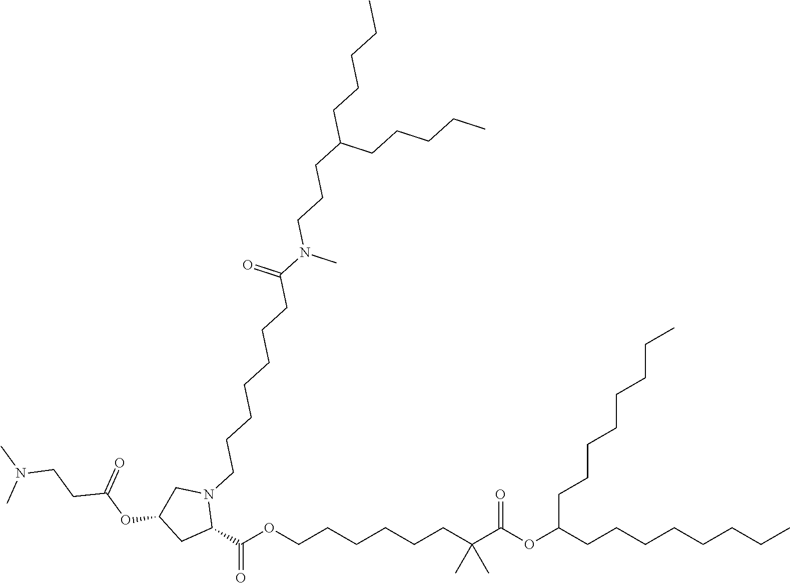 [SMILES: CCCCCCCCC(CCCCCCCC)OC(=O)C(C)(C)CCCCCCOC(=O)[C@@H]1C[C@H](OC(=O)CCN(C)C)CN1CCCCCCCC(=O)N(C)CCCC(CCCCC)CCCCC] |
| B5 | 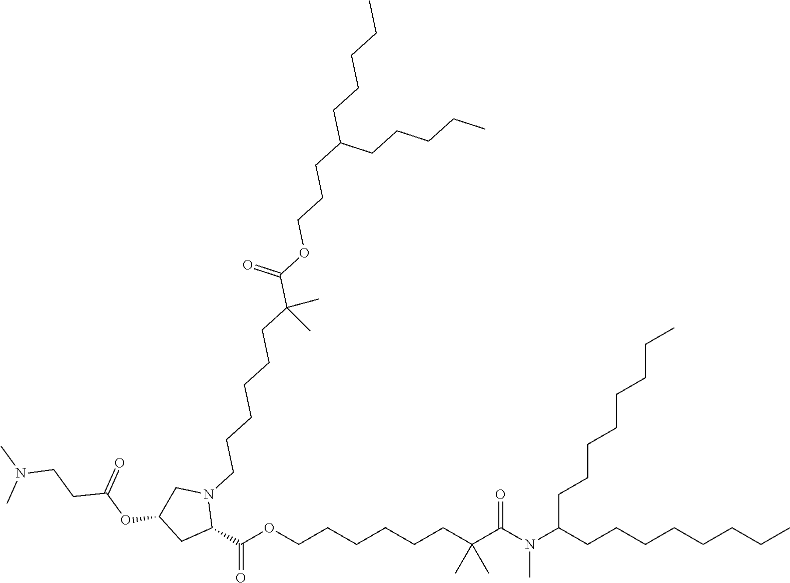 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCC(C)(C)C(=O)OCCCC(CCCCC)CCCCC] |
| B6 | 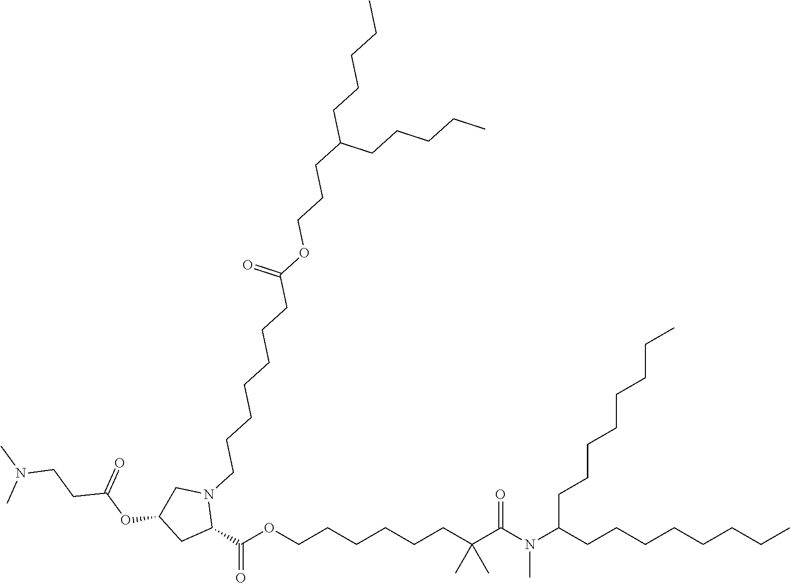 [SMILES: CCCCCCCCC(CCCCCCCC)N(C)C(=O)C(C)(C)CCCCCCOC(=O)[C@@H]1C[C@H](OC(=O)CCN(C)C)CN1CCCCCCCC(=O)OCCCC(CCCCC)CCCCC] |
| B7 | 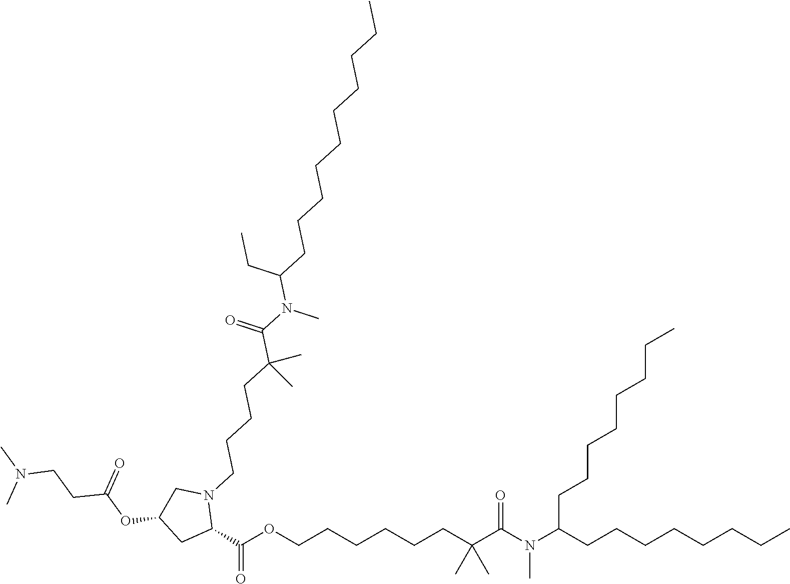 [SMILES: CCCCCCCCCCC(CC)N(C)C(=O)C(C)(C)CCCCN1C[C@@H](OC(=O)CCN(C)C)C[C@H]1C(=O)OCCCCCCC(C)(C)C(=O)N(C)C(CCCCCCCC)CCCCCCCC] |
| B8 | 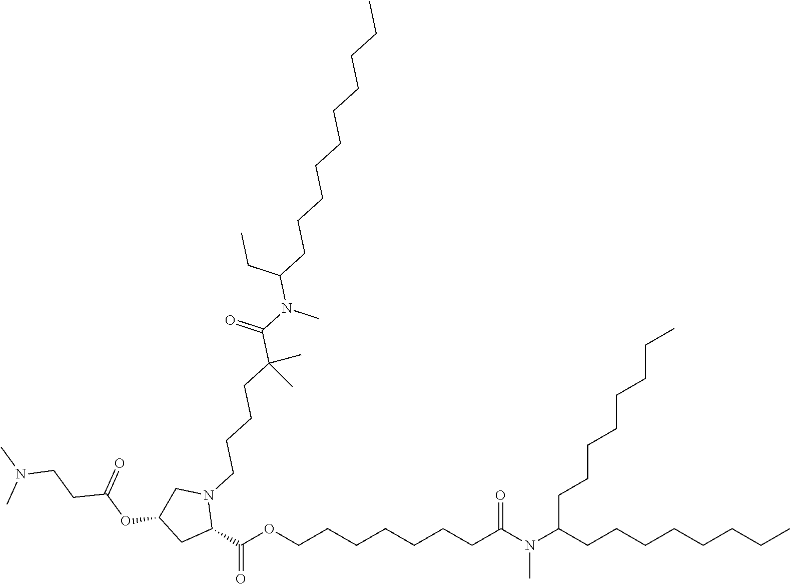 [SMILES: CCCCCCCCCCC(CC)N(C)C(=O)C(C)(C)CCCCN1C[C@@H](OC(=O)CCN(C)C)C[C@H]1C(=O)OCCCCCCCC(=O)N(C)C(CCCCCCCC)CCCCCCCC] |
| B9 | 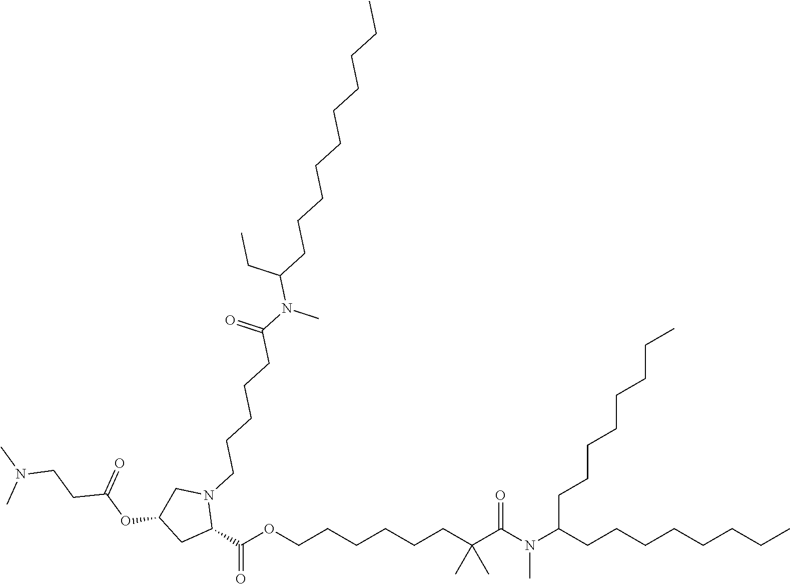 [SMILES: CCCCCCCCCCC(CC)N(C)C(=O)CCCCCN1C[C@@H](OC(=O)CCN(C)C)C[C@H]1C(=O)OCCCCCCC(C)(C)C(=O)N(C)C(CCCCCCCC)CCCCCCCC] |
| B10 | 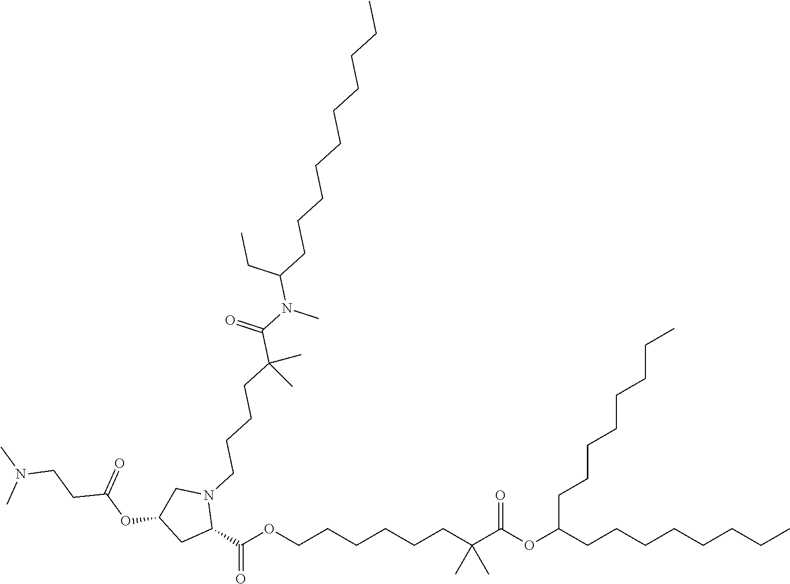 [SMILES: CCCCCCCCCCC(CC)N(C)C(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] |
| B11 | 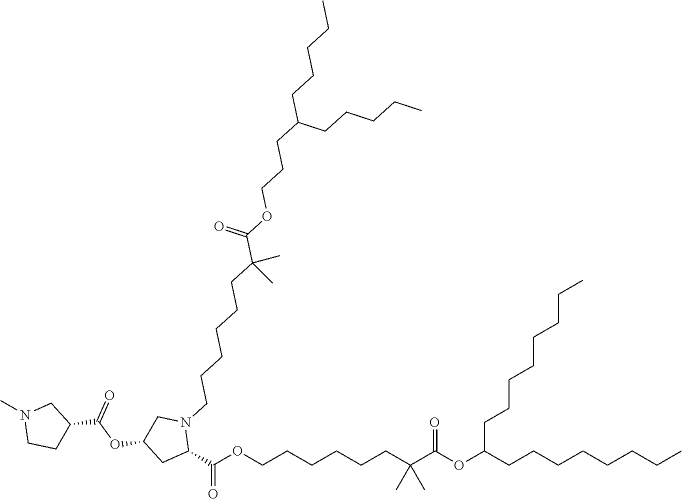 [SMILES: CCCCCCCCC(CCCCCCCC)OC(=O)C(C)(C)CCCCCCOC(=O)[C@@H]1C[C@H](OC(=O)[C@@H]2CCN(C)C2)CN1CCCCCCC(C)(C)C(=O)OCCCC(CCCCC)CCCCC] |
| B12 | 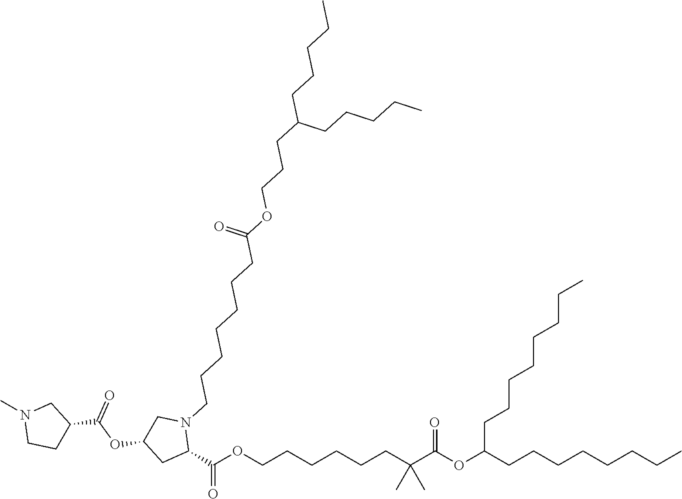 [SMILES: CCCCCCCCC(CCCCCCCC)OC(=O)C(C)(C)CCCCCCOC(=O)[C@@H]1C[C@H](OC(=O)[C@@H]2CCN(C)C2)CN1CCCCCCCC(=O)OCCCC(CCCCC)CCCCC] |
| B13 | 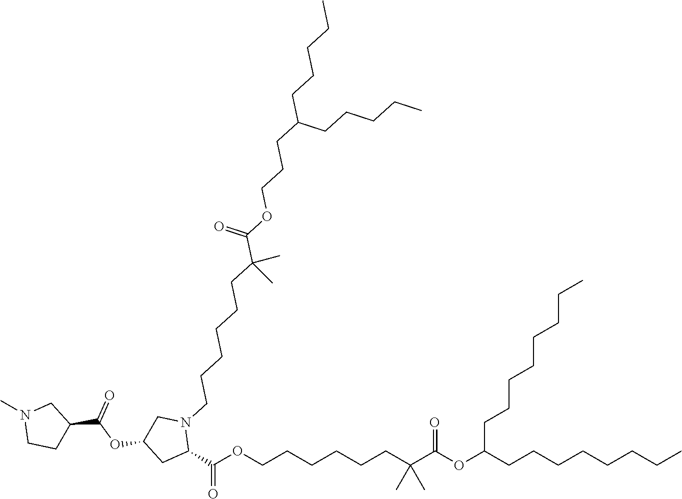 [SMILES: CCCCCCCCC(CCCCCCCC)OC(=O)C(C)(C)CCCCCCOC(=O)[C@@H]1C[C@H](OC(=O)[C@H]2CCN(C)C2)CN1CCCCCCC(C)(C)C(=O)OCCCC(CCCCC)CCCCC] |
| B14 | 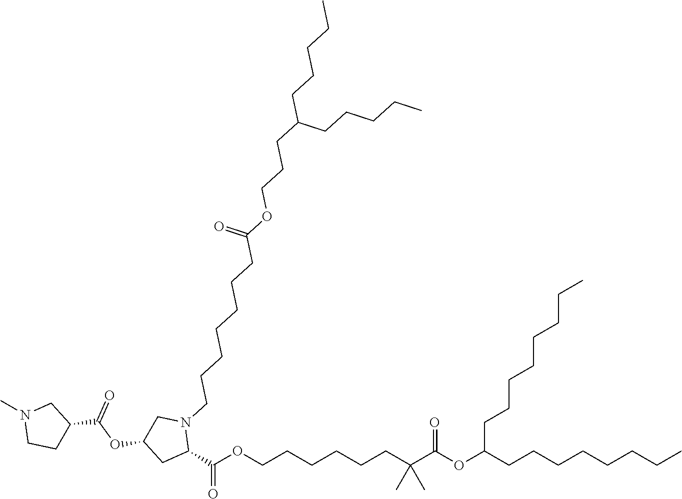 [SMILES: CCCCCCCCC(CCCCCCCC)OC(=O)C(C)(C)CCCCCCOC(=O)[C@@H]1C[C@H](OC(=O)[C@@H]2CCN(C)C2)CN1CCCCCCCC(=O)OCCCC(CCCCC)CCCCC] |
| B15 | 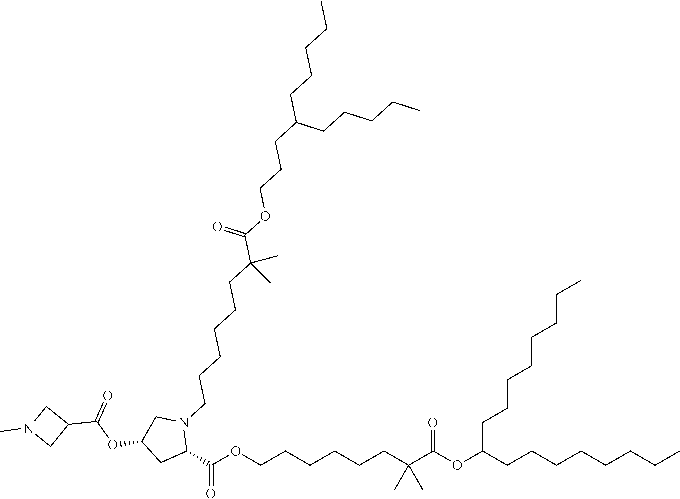 [SMILES: CCCCCCCCC(CCCCCCCC)OC(=O)C(C)(C)CCCCCCOC(=O)[C@@H]1C[C@H](OC(=O)C2CN(C)C2)CN1CCCCCCC(C)(C)C(=O)OCCCC(CCCCC)CCCCC] |
| B16 | 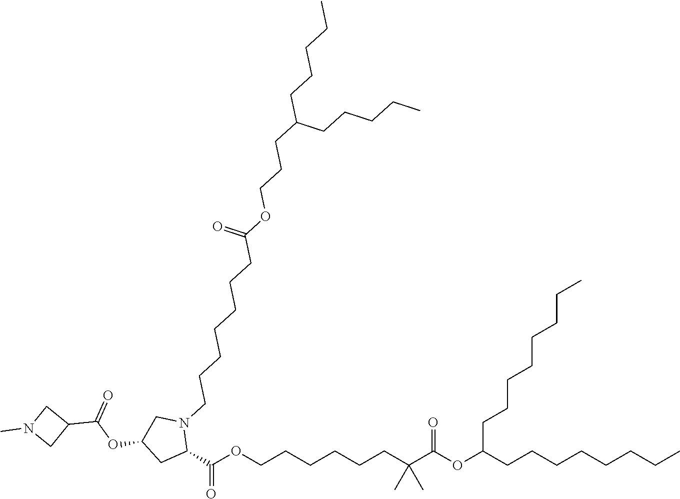 [SMILES: CCCCCCCCC(CCCCCCCC)OC(=O)C(C)(C)CCCCCCOC(=O)[C@@H]1C[C@H](OC(=O)C2CN(C)C2)CN1CCCCCCCC(=O)OCCCC(CCCCC)CCCCC] |
| B17 | 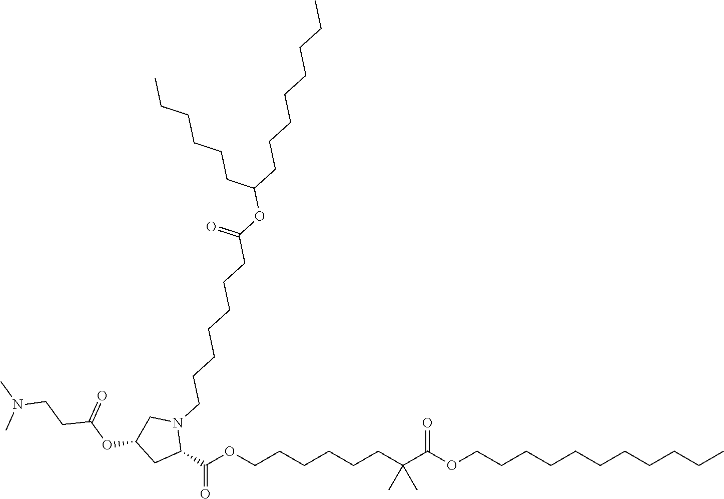 [SMILES: CCCCCCCCCCCOC(=O)C(C)(C)CCCCCCOC(=O)[C@@H]1C[C@H](OC(=O)CCN(C)C)CN1CCCCCCCC(=O)OC(CCCCCC)CCCCCCCC] |
| B18 | 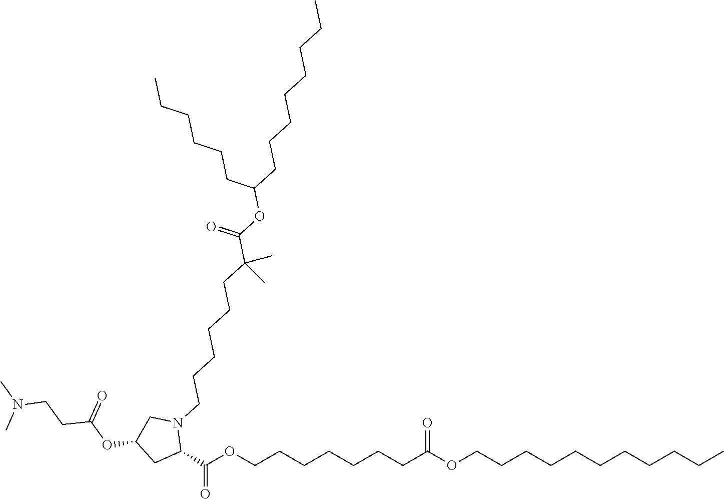 [SMILES: CCCCCCCCCCCOC(=O)CCCCCCCOC(=O)[C@@H]1C[C@H](OC(=O)CCN(C)C)CN1CCCCCCC(C)(C)C(=O)OC(CCCCCC)CCCCCCCC] |
| B19 | 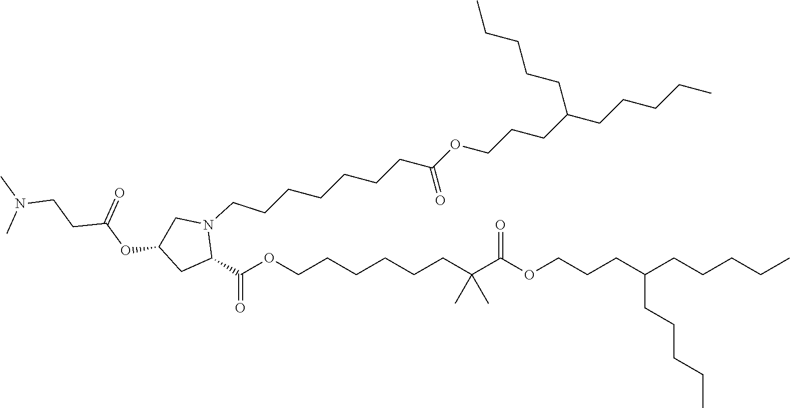 [SMILES: CCCCCC(CCCCC)CCCOC(=O)CCCCCCCN1C[C@@H](OC(=O)CCN(C)C)C[C@H]1C(=O)OCCCCCCC(C)(C)C(=O)OCCCC(CCCCC)CCCCC] |
| B20 | 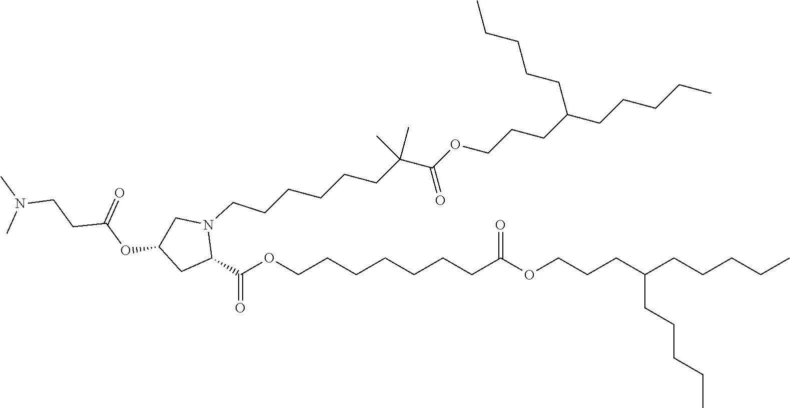 [SMILES: CCCCCC(CCCCC)CCCOC(=O)CCCCCCCOC(=O)[C@@H]1C[C@H](OC(=O)CCN(C)C)CN1CCCCCCC(C)(C)C(=O)OCCCC(CCCCC)CCCCC] |
| B21 | 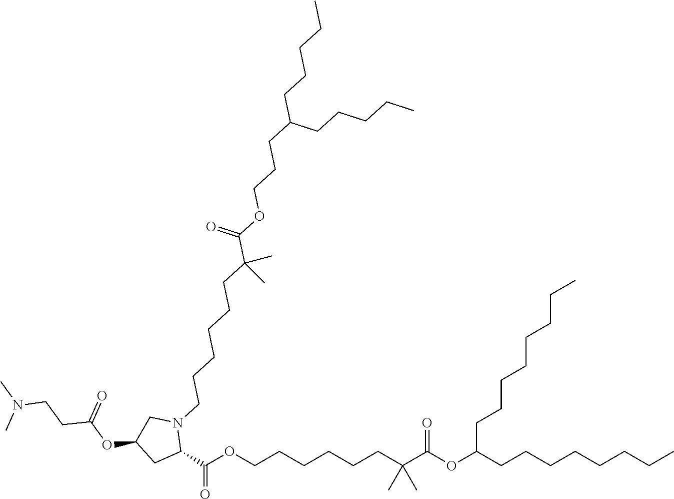 [SMILES: CCCCCCCCC(CCCCCCCC)OC(=O)C(C)(C)CCCCCCOC(=O)[C@@H]1C[C@@H](OC(=O)CCN(C)C)CN1CCCCCCC(C)(C)C(=O)OCCCC(CCCCC)CCCCC] |
| B22 | 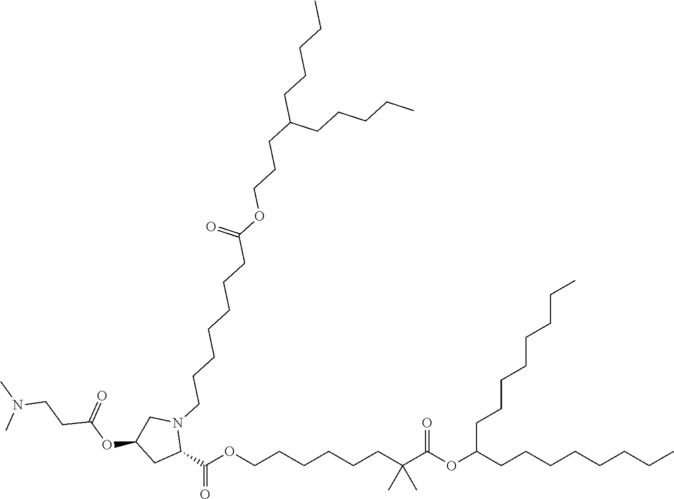 [SMILES: CCCCCCCCC(CCCCCCCC)OC(=O)C(C)(C)CCCCCCOC(=O)[C@@H]1C[C@@H](OC(=O)CCN(C)C)CN1CCCCCCCC(=O)OCCCC(CCCCC)CCCCC] |
| B23 | 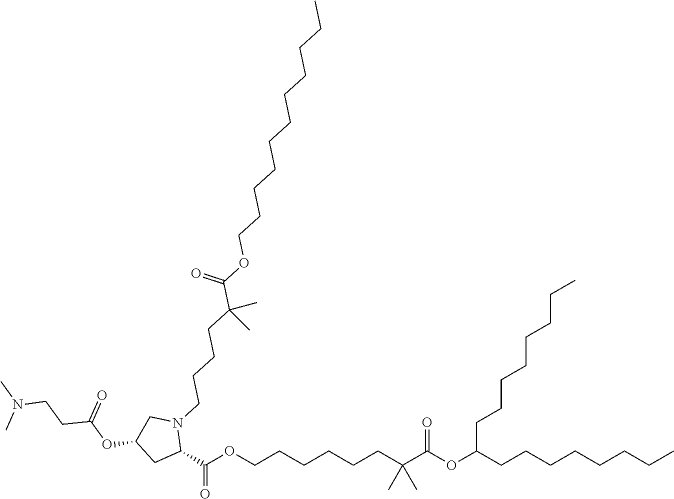 [SMILES: CCCCCCCCCCCOC(=O)C(C)(C)CCCCN1C[C@@H](OC(=O)CCN(C)C)C[C@H]1C(=O)OCCCCCCC(C)(C)C(=O)OC(CCCCCCCC)CCCCCCCC] |
| B24 | 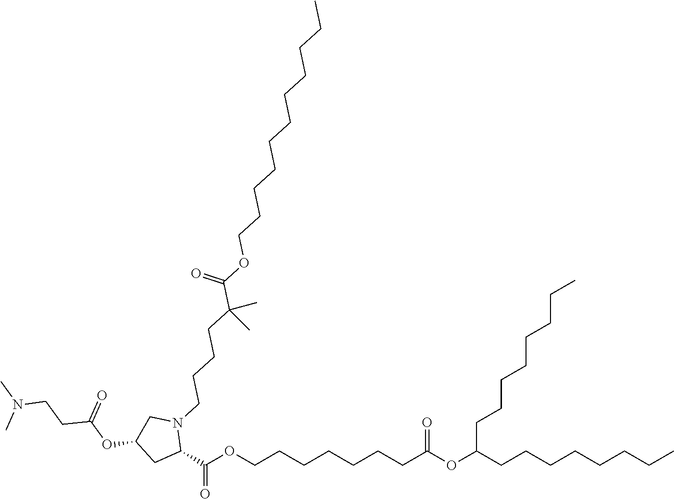 [SMILES: CCCCCCCCCCCOC(=O)C(C)(C)CCCCN1C[C@@H](OC(=O)CCN(C)C)C[C@H]1C(=O)OCCCCCCCC(=O)OC(CCCCCCCC)CCCCCCCC] |
| B25 | 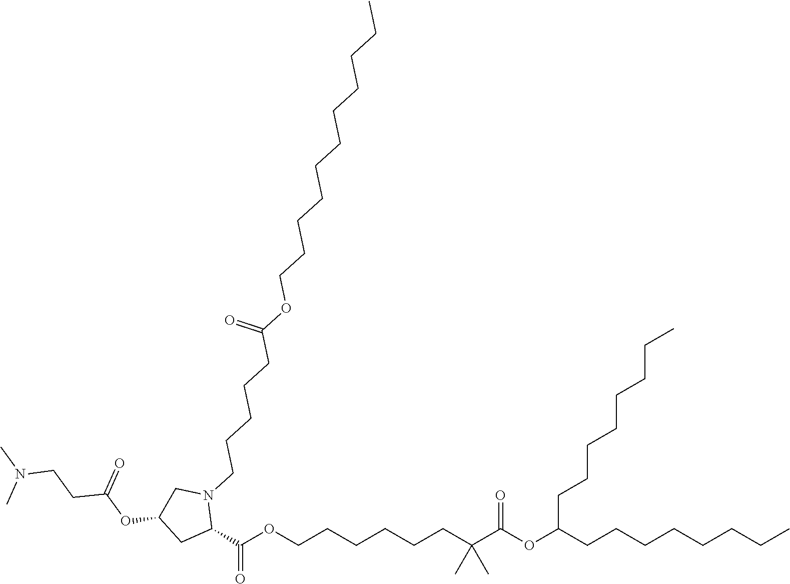 [SMILES: CCCCCCCCCCCOC(=O)CCCCCN1C[C@@H](OC(=O)CCN(C)C)C[C@H]1C(=O)OCCCCCCC(C)(C)C(=O)OC(CCCCCCCC)CCCCCCCC] |

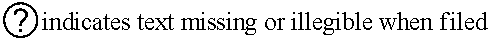

72. (canceled)

73. A lipid comprising at least two lipophilic tail groups and a head group of formula (G-HC-IIID): 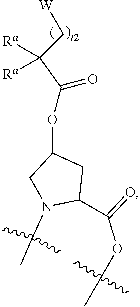
a pharmaceutically acceptable salt thereof, or a stereoisomer of any of the foregoing,
wherein:
R^{a} is each independently C₁-C₅alkyl, C₂-C₅ alkenyl, or C₂-C₅ alkynyl;
t2 is an integer from 0 to 5;
W is hydroxyl, substituted or unsubstituted hydroxyalkyl, or one of the following moieties: 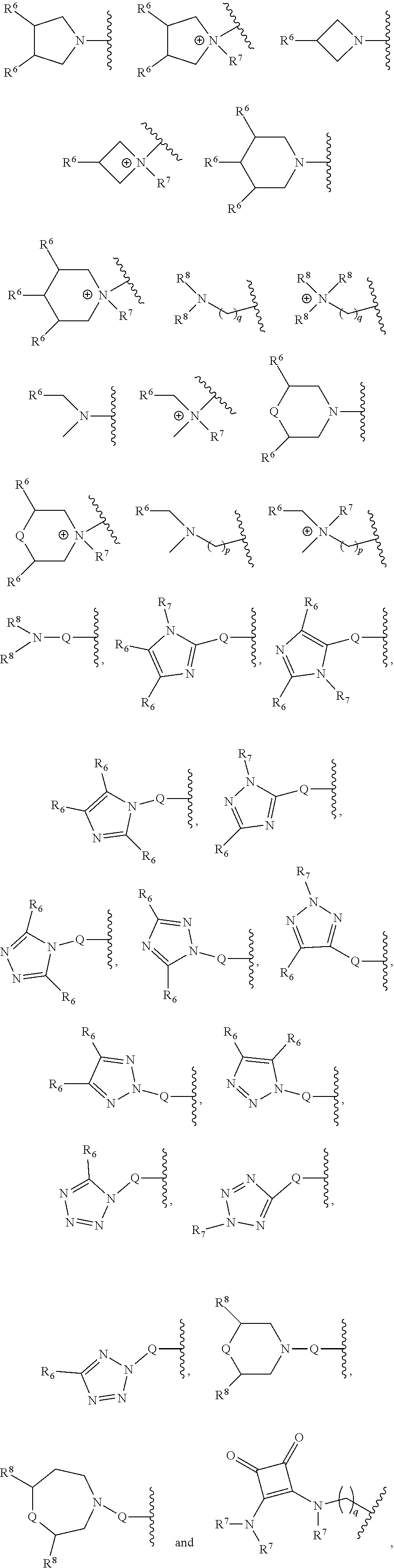
wherein
each Q is independently absent, —O—, —C(O)—, —C(S)—, —C(O)O—, —C(R⁷)₂—, —C(O)N(R⁷)—, —C(S)N(R⁷)—, or —N(R⁷);
R⁶ is independently H, alkyl, hydroxyl, hydroxyalkyl, alkoxy, —O-alkylene-O-alkyl, —O-alkylene-N(R⁷)₂, amino, alkylamino, aminoalkyl, thiol, thiolalkyl, or N⁺(R⁷)₃-alkylene-Q-;
each R⁸ is independently H, alkyl, hydroxyalkyl, amino, aminoalkyl, alkylamino, thiol, thiolalkyl, heterocyclyl, heteroaryl; or two R⁸ together with the nitrogen atom form a ring, optionally substituted with one or more alkyl, hydroxy, hydroxyalkyl, alkoxy, alkylaminoalkyl, alkylamino, or aminoalkyl;
q is 0, 1, 2, 3, 4, or 5; and
p is 0, 1, 2, 3, 4, or 5; and [symbol]
represents the bond connecting the head group to the tail groups.

74-79. (canceled)

80. An LNP composition comprising the lipid of claim 1, and one or more lipid components different than the lipid.

81-82. (canceled)

83. The LNP composition of claim 80, wherein the lipid component comprises one or more of a sterol, a PEG lipid, a phospholipid, and a neutral lipid.

84-91. (canceled)

92. A pharmaceutical composition comprising a composition of claim 1; a therapeutic agent selected from the group consisting of a nucleic acid molecule, a protein, and a small molecule drug; and a pharmaceutically acceptable excipient.

93-97. (canceled)

98. The pharmaceutical composition of claim 92, wherein the therapeutic agent is an RNA or DNA component.
